SEQUENCE LISTING (169 sequences)

SEQ ID NO: 1 - protein, 475 aa, Arabidopsis thaliana
  AtAAP8Ler
MDAYNNPSAVESGDAAVKSVDDDGREKRTGTFWTASAHIITAVIGSGVLSLAWAIAQLGWVAGTTVLVAFAIITYYTSTLLADCYRSPDSITGTRNYNYMGVVRSYLGGKKVQLCGVAQYVNLVGVTIGYTITASISLVAIGKSNCYHDKGHKAKCSVSNYPYMAAFGIVQIILSQLPNFHKLSFLSIIAAVMSFSYASIGIGLAIATVASGKIGKTELTGTVIGVDVTASEKVWKLFQAIGDIAFSYAFTTILIEIQDTLRSSPPENKVMKRASLAGVSTTTVFYILCGCIGYAAFGNQAPGDFLTDFGFYEPYWLIDFANACIALHLIGAYQVYAQPFFQFVEENCNKKWPQSNFINKEYSSKVPLLGKCRVNLFRLVWRTCYVVLTTFVAMIFPFFNAILGLLGAFVFWPLTVYFPVAMHIAQAKVKKYSRRWLALNLLVLVCLIVSALAAVGSIIGLINSVKSYKPFKNLD

SEQ ID NO: 2 - protein, 475 aa, Arabidopsis thaliana
  AtAAP8Cvi
MDAYNNPSAVESGDAAVKSVDDDGREKRTGTFWTASAHIITAVIGSGVLSLAWAIAQLGWVAGTTVLVAFAIITYYTSTLLADCYRSPDSITGTRNYNYMGVVRSYLGGKKVQLCGVAQYVNLVGVTIGYTITASISLVAIGKSNCYHDKGHKAKCSVSNYPYMAAFGIVQIILSQLPNFHKLSFLSIIAAVMSFSYASIGIGLAIATVASGKIGKTELTGTVIGVDVTASEKVWKLFQAIGDIAFSYAFTTILIEIQDTLRSSPPENKVMKRASLVGVSTTTVFYILCGCIGYAAFGNQAPGDFLTDFGFYEPYWLIDFANACIALHLIGAYQVYAQPFFQFVEENCNKKWPQSNFINKEYSSKVPLLGKCRINLFRLVWRTCYVVLTTFVAMIFPFFNAILGLLGAFAFWPLTVYFPVAMHIAQAKVKKYSRRWLALNLLVLVCLIVSALAAVGSIIGLINSVKSYKPFKNLD

SEQ ID NO: 3 - protein, 475 aa, Arabidopsis thaliana
  AtAAP8Col-0
MDAYNNPSAVESGDAAVKSVDDDGREKRTGTFWTASAHIITAVIGSGVLSLAWAIAQLGWVAGTTVLVAFAIITYYTSTLLADCYRSPDSITGTRNYNYMGVVRSYLGGKKVQLCGVAQYVNLVGVTIGYTITASISLVAIGKSNCYHDKGHKAKCSVSNYPYMAAFGIVQIILSQLPNFHKLSFLSIIAAVMSFSYASIGIGLAIATVASGKIGKTELTGTVIGVDVTASEKVWKLFQAIGDIAFSYAFTTILIEIQDTLRSSPPENKVMKRASLVGVSTTTVFYILCGCIGYAAFGNQAPGDFLTDFGFYEPYWLIDFANACIALHLIGAYQVYAQPFFQFVEENCNKKWPQSNFINKEYSSKVPLLGKCRVNLFRLVWRTCYVVLTTFVAMIFPFFNAILGLLGAFAFWPLTVYFPVAMHIAQAKVKKYSRRWLALNLLVLVCLIVSALAAVGSIIGLINSVKSYKPFKNLD

SEQ ID NO: 4 - protein, 475 aa, Arabidopsis thaliana
  AtAAP8 A410
MDAYNNPSAVESGDAAVKSVDDDGREKRTGTFWTASAHIITAVIGSGVLSLAWAIAQLGWVAGTTVLVAFAIITYYTSTLLADCYRSPDSITGTRNYNYMGVVRSYLGGKKVQLCGVAQYVNLVGVTIGYTITASISLVAIGKSNCYHDKGHKAKCSVSNYPYMAAFGIVQIILSQLPNFHKLSFLSIIAAVMSFSYASIGIGLAIATVASGKIGKTELTGTVIGVDVTASEKVWKLFQAIGDIAFSYAFTTILIEIQDTLRSSPPENKVMKRASLAGVSTTTVFYILCGCIGYAAFGNQAPGDFLTDFGFYEPYWLIDFANACIALHLIGAYQVYAQPFFQFVEENCNKKWPQSNFINKEYSSKVPLLGKCRVNLFRLVWRTCYVVLTTFVAMIFPFFNAILGLLGAFAFWPLTVYFPVAMHIAQAKVKKYSRRWLALNLLVLVCLIVSALAAVGSIIGLINSVKSYKPFKNLD

SEQ ID NO: 5 - DNA, 3359 bp, Arabidopsis thaliana
  AtAAP8Ler
agggagtactctaataagacgacctctgtcaataactctcttcccctctcttctctcctctggttcagtggttctctcacaatgatggacgcataccacaatccttcggcggtggagtcgggtgacgccgccgtgaaaagcgtcgacgacgatggtcgagagaagagaacgggaacattttggacggcgagtgcgcacataatcacggcggtcataggctcaggggtgctgtcgttggcttgggctatagcacagcttggttgggtggcaggaaccacagttttggtcgctttcgccatcattacttactacacgtccaccttgctcgccgactgttaccgttcgccggactccatcaccggaacacgcaactataattacatgggcgtcgtccgatcttaccttggtatggattcatataaacaaattcattttgtgtctttatcagcattgtttttcacagatttttcagttttctagacattttttctcagatgaacaaggattttgttcatttgatatcatttagattttgcctaactagtctcaatttacgacatgtgttttgattttcttccatttctgtcacaatgatgatggctggcgaaagaaaaaaaatctgatctaaaaatatatatttaatgctaagttggaatttgtaaatctacagtataattggctcatttcaacaatttctttccatgtaaatttgttgaagaacattattgttgttgaacaatgaaagaaaaaaatatggttgttagaaaaaaatgatttacgattttgccaagtgtgcatgctctttcatgggaagatatgaattaattatcaaaatctatataaaaaaaaggaagataatcttcattctttcataacttagttaataaattaaattgattaggattggtaacatagtcaattcaatttatcccgttaaagaatgttataaattcgattgttgacccctcgttgaaaatttggaattatgcgggatgtttagaaactttgccataagaccaaaagattggtagtatttgatagtagtacaagagtaatcatttttcttctttaataacataaaacgcaggtggtaaaaaggttcagctatgtggagtggcacagtacgtgaatctcgtaggggtcactattggttacaccatcactgcctccataagcttagtgtaagtcaaagattctgatttatttcgattattttgttatggttatactaacatgttgttctgaataaaattactaataattgtttgattggtgtttttgtacgtcttcgttagagcgattgggaaatcaaattgttatcatgacaagggacataaagcgaaatgttctgtatcgaattatccatacatggcggcatttgggatcgtccagatcattctgagccagcttcctaacttccacaagctctctttcctatccatcatcgccgcggttatgtccttctcttatgcgtctatcggaataggcctagccatcgctactgtagcaagtacattccccttctttatcttaaaacatagtggtttatatggatgattcttcaaagttgacactaaccgtgaaaatggtatacaatatatatgaaaggtgggaagattggtaagacagaattgacagggacagtgataggtgtggacgtaactgcgtctgaaaaagtttggaaattgtttcaagcgattggagacattgccttttcatacgcttttaccactattctcatcgagattcaggcatgtactactgattcctactatcttccgtttactattgttttcatttgcttgttattattaatttcgccaaaaagaggtaaaataagaataccttgaagataagatgttattattgattagaaaggtaggaaaaaatatagatggatggatgatggatcaaatagtttcatattttagatatgtgaagctctaaagatagtgacgctctagtagtatgtcttgtttattttgcaggacacattgagatcaagcccaccagagaacaaagtgatgaaacgagcaagtcttgccggagtctcaaccacaactgttttctacatcttgtgtggttgcatcggatatgctgcgttcggcaaccaagcccctggtgacttccttaccgattttggtttttacgaaccttattggctcatcgactttgccaatgcttgcattgctctccatctaatcggtgcctatcaggtataactcacaaacaaaagaataggataagtgtgtaacatacatttaccgtgttcaagttcattaaaagtctcattattgtgttagaatttttagctttaacaattcagaagattgtagaaatggagttattactaaatattgtttctaaaaaatgctctttttttttttttatccctgtattattcgcaggtgtatgcgcagccgtttttccagtttgttgaggaaaactgcaacaaaaaatggcctcaaagcaatttcatcaacaaagaatactcgtcaaaggttcctttgcttggaaaatgtcgtgtcaacctcttcagactggtttggaggacatgctatgttgttttgacaacatttgtagcaatgatattccccttcttcaatgcgatcttgggtttgctaggggcattcgtgttctggccactcacagtttattttccggtggcaatgcacattgcgcaggctaaagtcaagaagtattctcgtagatggttggccttgaacctcctcgtattggtttgcttgatcgtctcggcccttgccgccgtaggatccatcattggcttaattaatagtgtcaagtcatacaagcccttcaagaatttagactagtgtgacttataatctatgtttgccaaaaaaaaaccttgtgatccatatgaaatttatttcatgctaaatatttagtacttaatgtttctccaaataatgtgacgttctgttttcagctatgttaaaaaacaaaatgctaacttgtgtattagtactaaaatttatgaaaatgtattagttattgatttatttttaggactacaattattgaatcaacattggatgtttgagtcccatgagatatggatttcagcttttttcaaattcgtgtggttgtgtcaatttcgagttattattatttattttgcttaatggaattgtcggggaaatcttgaaaacagacactcacagattgtgtaatttatttggtttggtgtgtcctacataagttgctatcacatcttatgtattggaggagttgggcaatagaggatcaaggcaagtttggttttctattaacgtttctactctgcatttgcttacaaagtcattttcaaggttttgtggtcgtatgtcacttgatgg

SEQ ID NO: 6 - DNA, 3368 bp, Arabidopsis thaliana
  AtAAP8Cvi
agggagtactctaataagacgacctctgtcaataactctcttcccctctcttctctcctctggttcagtggttctctcacaatgatggacgcatacccaatccttcggcggtggagtcgggtgacgccgccgtgaaaagcgtcgacgacgatggtcgagagaagagaacgggaacattttggacggcgagtgcgcacataatcacggcggtcataggctcaggggtgctgtcgttggcttgggctatagcacagcttggttgggtggcaggaaccacagttttggtcgctttcgccatcattacttactacacgtccaccttgctcgccgactgttaccgttcgccggactccatcaccggaacacgcaactataattacatgggcgtcgtccgatcttaccttggtatggattcatataaacaaattcattttgtgtctttatcagcattgtttttcacagatttttcagttttctagacattttttctcagatgaacaaggattttgttcatttgatatcatttagattttgcctaactagtctcaatttaggacatgtgttttgattttcttccatttctgtcacaatgatgatggctggcgaaagaaaaaaaatctgatctaaaaatatatatttaatgctaagttggaatttgtaaatctacagtataattggctcatttcaacaattttttaccatgtaaatttgttgaagaacattattgttgttgaacaatgaaagaaaaaaatatggttgttagaaaaaaatgatttacgattttgccaagtgtgcatgctctttcatgggaagatatgaattaattatcaaaatctatataaaaaaaaggaagataatcttcattctttcataacttagttaataaattaaattgattaggattggtaacatagtcaattcaatttatcccgttaaagaatgttataaattcgattgttgacccctcgttgaaaatttggaattatgcgggatgtttagaaactttgccataagaccaaaagattggtagtatttgatagtagtacaagagtaatcatttttcttctttaataacataaaacgcaggtggtaaaaaggttcagctatgtggagtggcacagtacgtgaatctcgtaggggtcactattggttacaccatcactgcctccataagcttagtgtaagtcaaagattctgatttatttcgattattttgttatggttatactaacatgttgttctgaataaaattactaataattgtttgattggtgtttttgtacgtcttcgttagagcgattgggaaatcaaattgttatcatgacaagggacataaagcgaaatgttctgtatcgaattatccatacatggcggcatttgggatcgtccagatcattctgagccagcttcctaacttccacaagctctctttcctatccatcatcgccgcggttatgtccttctcttatgcgtctatcggaataggcctagccatcgctactgtagcaagtacattccccttctttatcttaaaacatagtggtttatatggatgattcttcaaagttgacactaaccgtgaaaatggtatacaatatatatgaaaggtgggaagattggtaagacagaattgacagggacagtgataggtgtggacgtaactgcgtctgaaaaagtttggaaattgtttcaagcgattggagacattgccttttcatacgcttttaccactattctcatcgagattcaggcatgtactactgattcctactatcttccgtttactattgttttcatttgcttgttattattaatttcgccaaagagaggtaaaataagaataccttgaagataagatgttattattaattagacagttaggaaaaaatatagatggatggatgatggataaaaatagtttcatattttagatatgtgaagctctaaagatagtgacgctctagtagtatgtcttgtttattttgcaggacacattgagatcaagcccaccagagaacaaagtgatgaaacgagcaagtcttgtcggagtctcaaccacaactgttttctacatcttgtgtggttgcatcggatatgctgcgttcggcaaccaagcccctggtgacttccttaccgattttggtttttacgaaccttattggctcatcgactttgccaatgcttgcattgctctccatctaataggtgcctatcaggtataactcacaaacaaaagaataggataagtgtgtaacatacatttaccgtgttcaagttcattaaaagtctcattattgtgttagaatttttagctttaacaattcagaagattgtagaaatggagttattactaaatattgtttctaaaaaatgctctttttttttttttatccctgtattattcgcaggtgtatgcgcagccgtttttccagtttgttgaggaaaactgcaacaaaaaatggcctcaaagcaatttcatcaacaaagaatactcgtcaaaggttcctttgcttggaaaatgtcgtatcaacctcttcagactggtttggaggacatgctatgttgttttgacaacatttgtagcaatgatattccccttcttcaatgcgatcttgggtttgctaggggcactcgcgttctggccactcacagtttattttccggtggcaatgcacattgcgcaggctaaagtcaagaagtattctcgtagatggttggccttgaacctcctcgtattggtttgcttgatcgtctcggcccttgccgccgtaggatccatcattggcttaattaatagtgtcaagtcatacaagcccttcaagaatttagactagtgtgacttataatctatgtttgccaaaaaaaaaccttgtgatccatatgaattatatgaaatttatttgatgctaaatatttagtacttaatgtttctccaaataatgtgacgttctgttttcagctatgttaaaaaccaaaatgctaacttatgtattagtactaaaatttatgaaaatgtattagttattgatttatttttaggactacaattattgaatcaacattggatgtttgagtcccatgagatatggatttcagcttttttcaaattcgtgtggttgtgtcaatttcgagttattattatttattttgcttaatggaattgtcggggaaatcttgaaaacagacactcacagattgtgtaatttatttggtttggtgtgtcctacataagttgctatcacatcttatgtattggaggagttgggcaatagaggatcaaggcaagtttggttttctattaacgtttctactctgcatttgcttacaaagtcattttcaaggttttgtggtcgtatgtcacttgatgg

SEQ ID NO: 7 - DNA, 3369 bp, Arabidopsis thaliana
  AtAAP8Col-0
agggagtactctaataagacgacctctgtcaataactctcttcccctctcttctctcctctggttcagtggttctctcacaatgatggacgcatacaacaatccctcggcggtggagtcgggtgacgccgccgtgaaaagcgtcgacgacgatggtcgagagaagagaacgggaacattttggacggcgagtgcgcacataatcacggcggtcataggctcaggggtgctgtcgttggcttgggctatagcacagcttggttgggtggcaggaaccacagttttggtcgctttcgccatcattacttactacacgtccaccttgctcgccgactgttaccgttcgccggactccatcaccggaacacgcaactataattacatgggcgtcgtccgatcttaccttggtatggattcatataaacaaattcattttgtgtctttatcagcattgtttttcacagatttttcagttttctagacattttttctcagatgaacaaggattttgttcatttgatatcatttagattttgcctaactagtctcaatttaggacatgtgttttgattttcttccatttctgtcacaatgatgatggctggcgaaagaaaaaaaatctgatctaaaaatatatatttaatgctaagttggaatttgtaaatctacagtataattggctcatttcaacaattttttaccatgtaaatttgttgaagaacattattgttgttgaacaatgaaagaaaaaaatatggttgttagaaaaaaatgatttacgattttgccaagtgtgcatgctctttcatgggaagatatgaattaattatcaaaatctatataaaaaaaaggaagataatcttcattctttcataacttagttaataaattaaattgattaggattggtaacatagtcaattcaatttatcccgttaaagaatgttataaattcgattgttgacccctcgttgaaaatttggaattatgcgggatgtttagaaactttgccataagaccaaaagattggtagtatttgatagtagtacaagagtaatcatttttcttctttaataacataaaacgcaggtggtaaaaaggttcagctatgtggagtggcacagtacgtgaatctcgtaggggtcactattggttacaccatcactgcctccataagcttagtgtaagtcaaagattctgatttatttcgattattttgttatggttatactaacatgttgttctgaataaaattactaataattgtttgattggtgtttttgtacgtcttcgttagagcgattgggaaatcaaattgttatcatgacaagggacataaagcgaaatgttctgtatcgaattatccatacatggcggcatttgggatcgtccagatcattctgagccagcttcctaacttccacaagctctctttcctatccatcatcgccgcggttatgtccttctcttatgcgtctatcggaataggcctagccatcgctactgtagcaagtacattccccttctttatcttaaaacatagtggtttatatggatgattcttcaaagttgacactaaccgtgaaaatggtatacaatatatatgaaaggtgggaagattggtaagacagaattgacagggacagtgataggtgtggacgtaactgcgtctgaaaaagtttggaaattgtttcaagcgattggagacattgccttttcatacgcttttaccactattctcatcgagattcaggcatgtactactgattcctactatcttccgtttactattgttttcatttgcttgttattattaatttcgccaaagagaggtaaaataagaataccttgaagataagatgttattattaattagacagttaggaaaaaatatagatggatggatgatggataaaaatagtttcatattttagatatgtgaagctctaaagatagtgacgctctagtagtatgtcttgtttattttgcaggacacattgagatcaagcccaccagagaacaaagtgatgaaacgagcaagtcttgtcggagtctcaaccacaactgttttctacatcttgtgtggttgcatcggatatgctgcgttcggcaaccaagcccctggtgacttccttaccgattttggtttttacgaaccttattggctcatcgactttgccaatgcttgcattgctctccatctaataggtgcctatcaggtataactcacaaacaaaagaataggataagtgtgtaacatacatttaccgtgttcaagttcattaaaagtctcattattgtgttagaatttttagctttaacaattcagaagattgtagaaatggagttattactaaatattgtttctaaaaaatgctctttttttttttttatccctgtattattcgcaggtgtatgcgcagccgtttttccagtttgttgaggaaaactgcaacaaaaaatggcctcaaagcaatttcatcaacaaagaatactcgtcaaaggttcctttgcttggaaaatgtcgtgtcaacctcttcagactggtttggaggacatgctatgttgttttgacaacatttgtagcaatgatattccccttcttcaatgcgatcttgggtttgctaggggcattcgcgttctggccactcacagtttattttccggtggcaatgcacattgcgcaggctaaagtcaagaagtattctcgtagatggttggccttgaacctcctcgtattggtttgcttgatcgtctcggcccttgccgccgtaggatccatcattggcttaattaatagtgtcaagtcatacaagcccttcaagaatttagactagtgtgacttataatctatgtttgccaaaaaaaaaccttgtgatccatatgaattatatgaaatttatttcatgctaaatatttagtacttaatgtttctccaaataatgtgacgttctgttttcagctatgttaaaaaccaaaatgctaacttatgtattagtactaaaatttatgaaaatgtattagttattgatttatttttaggactacaattattgaatcaacattggatgtttgagtcccatgagatatggatttcagcttttttcaaattcgtgtggttgtgtcaatttcgagttattattatttattttgcttaatggaattgtcggggaaatcttgaaaacagacactcacagattgtgtaatttatttggtttggtgtgtcctacataagttgctatcacatcttatgtattggaggagttgggcaatagaggatcaaggcaagtttggttttctattaacgtttctactctgcatttgcttacaaagtcattttcaaggttttgtggtcgtatgtcacttgatgg

SEQ ID NO: 8 - DNA, 3359 bp, Arabidopsis thaliana
  AtAAP8 A410
agggagtactctaataagacgacctctgtcaataactctcttcccctctcttctctcctctggttcagtggttctctcacaatgatggacgcataccacaatccttcggcggtggagtcgggtgacgccgccgtgaaaagcgtcgacgacgatggtcgagagaagagaacgggaacattttggacggcgagtgcgcacataatcacggcggtcataggctcaggggtgctgtcgttggcttgggctatagcacagcttggttgggtggcaggaaccacagttttggtcgctttcgccatcattacttactacacgtccaccttgctcgccgactgttaccgttcgccggactccatcaccggaacacgcaactataattacatgggcgtcgtccgatcttaccttggtatggattcatataaacaaattcattttgtgtctttatcagcattgtttttcacagatttttcagttttctagacattttttctcagatgaacaaggattttgttcatttgatatcatttagattttgcctaactagtctcaatttacgacatgtgttttgattttcttccatttctgtcacaatgatgatggctggcgaaagaaaaaaaatctgatctaaaaatatatatttaatgctaagttggaatttgtaaatctacagtataattggctcatttcaacaatttctttccatgtaaatttgttgaagaacattattgttgttgaacaatgaaagaaaaaaatatggttgttagaaaaaaatgatttacgattttgccaagtgtgcatgctctttcatgggaagatatgaattaattatcaaaatctatataaaaaaaaggaagataatcttcattctttcataacttagttaataaattaaattgattaggattggtaacatagtcaattcaatttatcccgttaaagaatgttataaattcgattgttgacccctcgttgaaaatttggaattatgcgggatgtttagaaactttgccataagaccaaaagattggtagtatttgatagtagtacaagagtaatcatttttcttctttaataacataaaacgcaggtggtaaaaaggttcagctatgtggagtggcacagtacgtgaatctcgtaggggtcactattggttacaccatcactgcctccataagcttagtgtaagtcaaagattctgatttatttcgattattttgttatggttatactaacatgttgttctgaataaaattactaataattgtttgattggtgtttttgtacgtcttcgttagagcgattgggaaatcaaattgttatcatgacaagggacataaagcgaaatgttctgtatcgaattatccatacatggcggcatttgggatcgtccagatcattctgagccagcttcctaacttccacaagctctctttcctatccatcatcgccgcggttatgtccttctcttatgcgtctatcggaataggcctagccatcgctactgtagcaagtacattccccttctttatcttaaaacatagtggtttatatggatgattcttcaaagttgacactaaccgtgaaaatggtatacaatatatatgaaaggtgggaagattggtaagacagaattgacagggacagtgataggtgtggacgtaactgcgtctgaaaaagtttggaaattgtttcaagcgattggagacattgccttttcatacgcttttaccactattctcatcgagattcaggcatgtactactgattcctactatcttccgtttactattgttttcatttgcttgttattattaatttcgccaaaaagaggtaaaataagaataccttgaagataagatgttattattgattagaaaggtaggaaaaaatatagatggatggatgatggatcaaatagtttcatattttagatatgtgaagctctaaagatagtgacgctctagtagtatgtcttgtttattttgcaggacacattgagatcaagcccaccagagaacaaagtgatgaaacgagcaagtcttgccggagtctcaaccacaactgttttctacatcttgtgtggttgcatcggatatgctgcgttcggcaaccaagcccctggtgacttccttaccgattttggtttttacgaaccttattggctcatcgactttgccaatgcttgcattgctctccatctaatcggtgcctatcaggtataactcacaaacaaaagaataggataagtgtgtaacatacatttaccgtgttcaagttcattaaaagtctcattattgtgttagaatttttagctttaacaattcagaagattgtagaaatggagttattactaaatattgtttctaaaaaatgctctttttttttttttatccctgtattattcgcaggtgtatgcgcagccgtttttccagtttgttgaggaaaactgcaacaaaaaatggcctcaaagcaatttcatcaacaaagaatactcgtcaaaggttcctttgcttggaaaatgtcgtgtcaacctcttcagactggtttggaggacatgctatgttgttttgacaacatttgtagcaatgatattccccttcttcaatgcgatcttgggtttgctaggggcattcgcgttctggccactcacagtttattttccggtggcaatgcacattgcgcaggctaaagtcaagaagtattctcgtagatggttggccttgaacctcctcgtattggtttgcttgatcgtctcggcccttgccgccgtaggatccatcattggcttaattaatagtgtcaagtcatacaagcccttcaagaatttagactagtgtgacttataatctatgtttgccaaaaaaaaaccttgtgatccatatgaaatttatttcatgctaaatatttagtacttaatgtttctccaaataatgtgacgttctgttttcagctatgttaaaaaacaaaatgctaacttgtgtattagtactaaaatttatgaaaatgtattagttattgatttatttttaggactacaattattgaatcaacattggatgtttgagtcccatgagatatggatttcagcttttttcaaattcgtgtggttgtgtcaatttcgagttattattatttattttgcttaatggaattgtcggggaaatcttgaaaacagacactcacagattgtgtaatttatttggtttggtgtgtcctacataagttgctatcacatcttatgtattggaggagttgggcaatagaggatcaaggcaagtttggttttctattaacgtttctactctgcatttgcttacaaagtcattttcaaggttttgtggtcgtatgtcacttgatgg

SEQ ID NO: 9 - protein, 485 aa, Oryza sativa Indica Group
  EEC81471 OsI_24794
MERPQEKVATTTTAAFNLAESGYADRPDLDDDGREKRTGTLVTASAHIITAVIGSGVLSLAWAIAQLGWVIGPAVLVAFSVITWFCSSLLADCYRSPDPVHGKRNYTYGQAVRANLGVAKYRLCSVAQYVNLVGVTIGYTITTAISMGAIKRSNCFHRNGHDAACLASDTTNMIIFAGIQILLSQLPNFHKIWWLSIVAAVMSLAYSTIGLGLSIAKIAGGAHPEATLTGVTVGVDVSASEKIWRTFQSLGDIAFAYSYSNVLIEIQDTLRSSPAENEVMKKASFIGVSTTTTFYMLCGVLGYAAFGNRAPGNFLTGFGFYEPFWLVDVGNVCIVVHLVGAYQVFCQPIYQFAEAWARSRWPDSAFVNGERVLRLPLGAGDFPVSALRLVWRTAYVVLTAVAAMAFPFFNDFLGLIGAVSFWPLTVYFPVQMYMSQAKVRRFSPTWTWMNVLSLACLVVSLLAAAGSIQGLIKSVAHYKPFSVSS

SEQ ID NO: 10 - DNA, 1458 bp, Oryza sativa Indica Group
  EEC81471 OsI_24794
atggagaggccgcaagagaaggtggccaccaccaccaccgccgccttcaacctcgccgagtccggctacgccgaccgccccgacctcgacgacgacggccgcgagaagcgcacagggacgctggtgacggcgagcgcgcacataataacggcggtgatcggctccggcgtgctgtcgctggcgtgggcgatagcgcagctggggtgggtgatcgggccggccgtgctggtggcgttctcggtcataacctggttctgctccagcctcctcgccgactgctaccgatctcccgaccccgtccatggcaagcgcaactacacctacggccaagccgtcagggccaacctaggtgtggccaagtacaggctctgctcggtggcacagtacgtcaatctcgtcggcgtcaccattggctacaccatcactacggccatcagcatgggtgcgatcaaacggtccaactgcttccatcgcaacggccacgacgcagcctgcttggcatctgacacgaccaacatgatcatatttgctggcatccaaatcctcctctcgcagctgccgaattttcacaaaatttggtggctctccattgtcgctgctgtcatgtcactggcctactcaaccattggccttggcctctccattgcaaaaattgcaggtggggcccaccccgaggcaaccctcacaggggtgactgttggagtggatgtgtctgcaagtgagaaaatctggagaacttttcagtcacttggtgacattgcctttgcatactcctactccaatgtcctcatagaaattcaggacacgctgcggtcgagcccggcggagaacgaggtgatgaagaaggcgtcgttcatcggagtctcgacgacgacgacgttctacatgctgtgcggcgtgctcggctacgcggcgttcggcaaccgcgcgccggggaacttcctcaccggcttcggcttctacgagcccttctggctcgtcgacgtcggcaacgtctgcatcgtcgtccacctcgtcggcgcctaccaggtcttctgccagcccatctaccagttcgccgaggcctgggcgcgctcgcggtggccggacagcgccttcgtcaacggcgagcgcgtgctccggctgccgctcggcgccggcgacttccccgtcagcgcgctccgcctcgtctggcgcacggcctacgtcgtgctcaccgccgtcgccgccatggcgttccccttcttcaacgacttcctcggcctcatcggcgccgtctccttctggccgctcaccgtctacttccccgtccagatgtacatgtctcaggccaaggtccggcgattctcgccgacgtggacgtggatgaacgtgctcagcctcgcctgcctcgtcgtctccctcctcgccgccgccggctccatccagggcctcatcaaatccgtcgcacattacaagccattcagcgtctcctcatga

SEQ ID NO: 11 - protein, 485 aa, Oryza sativa Japonica Group
  EEE66520 OsJ_22995
MERPQEKVATTTTAAFNLAESGYADRPDLDDDGREKRTGTLVTASAHIITAVIGSGVLSLAWAIAQLGWVIGPAVLVAFSVITWFCSSLLADCYRSPDPVHGKRNYTYGQAVRANLGVAKYRLCSVAQYVNLVGVTIGYTITTAISMGAIKRSNWFHRNGHDAACLASDTTNMIIFAGIQILLSQLPNFHKIWWLSIVAAVMSLAYSTIGLGLSIAKIAGGAHPEATLTGVTVGVDVSASEKIWRTFQSLGDIAFAYSYSNVLIEIQDTLRSSPAENEVMKKASFIGVSTTTTFYMLCGVLGYAAFGNRAPGNFLTGFGFYEPFWLVDVGNVCIVVHLVGAYQVFCQPIYQFAEAWARSRWPDSAFVNGERVLRLPLGAGDFPVSALRLVWRTAYVVLTAVAAMAFPFFNDFLGLIGAVSFWPLTVYFPVQMYMSQAKVRRFSPTWTWMNVLSLACLVVSLLAAAGSIQGLIKSVAHYKPFSVSS

SEQ ID NO: 12 - DNA, 1458 bp, Oryza sativa Japonica Group
  EEE66520 OsJ_22995
atggagaggccgcaagagaaggtggccaccaccaccaccgccgccttcaacctcgccgagtccggctacgccgaccgccccgacctcgacgacgacggccgcgagaagcgcacagggacgctggtgacggcgagcgcgcacataataacggcggtgatcggctccggcgtgctgtcgctggcgtgggcgatagcgcagctggggtgggtgatcgggccggccgtgctggtggcgttctcggtcataacctggttctgctccagcctcctcgccgactgctaccgatctcccgaccccgtccatggcaagcgcaactacacctacggccaagccgtcagggccaacctaggtgtggccaagtacaggctctgctcggtggcacagtacgtcaatctcgtcggcgtcaccattggctacaccatcactacggccatcagcatgggtgcgatcaaacggtccaactggttccatcgcaacggccacgacgcagcctgcttggcatctgacacgaccaacatgatcatatttgctggcatccaaatcctcctctcgcagctgccgaattttcacaaaatttggtggctctccattgtcgctgctgtcatgtcactggcctactcaaccattggccttggcctctccattgcaaaaattgcaggtggggcccaccccgaggcaaccctcacaggggtgactgttggagtggatgtgtctgcaagtgagaaaatctggagaacttttcagtcacttggtgacattgcctttgcatactcctactccaatgtcctcatagaaattcaggacacgctgcggtcgagcccggcggagaacgaggtgatgaagaaggcgtcgttcatcggagtctcgacgacgacgacgttctacatgctgtgcggcgtgctcggctacgcggcgttcggcaaccgcgcgccggggaacttcctcaccggcttcggcttctacgagcccttctggctcgtcgacgtcggcaacgtctgcatcgtcgtccacctcgtcggcgcctaccaggtcttctgccagcccatctaccagttcgccgaggcctgggcgcgctcgcggtggccggacagcgccttcgtcaacggcgagcgcgtgctccggctgccgctcggcgccggcgacttccccgtcagcgcgctccgcctcgtctggcgcacggcctacgtcgtgctcaccgccgtcgccgccatggcgttccccttcttcaacgacttcctcggcctcatcggcgccgtctccttctggccgctcaccgtctacttccccgtccagatgtacatgtctcaggccaaggtccggcgattctcgccgacgtggacgtggatgaacgtgctcagcctcgcctgcctcgtcgtctccctcctcgccgccgccggctccatccagggcctcatcaaatccgtcgcacattacaagccattcagcgtctcctcatga

SEQ ID NO: 13 - protein, 487 aa, Oryza sativa Japonica Group
  XP_015647443 AAP6
MGMERPQEKVATTTTAAFNLAESGYADRPDLDDDGREKRTGTLVTASAHIITAVIGSGVLSLAWAIAQLGWVIGPAVLVAFSVITWFCSSLLADCYRSPDPVHGKRNYTYGQAVRANLGVAKYRLCSVAQYVNLVGVTIGYTITTAISMGAIKRSNWFHRNGHDAACLASDTTNMIIFAGIQILLSQLPNFHKIWWLSIVAAVMSLAYSTIGLGLSIAKIAGGAHPEATLTGVTVGVDVSASEKIWRTFQSLGDIAFAYSYSNVLIEIQDTLRSSPAENEVMKKASFIGVSTTTTFYMLCGVLGYAAFGNRAPGNFLTGFGFYEPFWLVDVGNVCIVVHLVGAYQVFCQPIYQFAEAWARSRWPDSAFVNGERVLRLPLGAGDFPVSALRLVWRTAYVVLTAVAAMAFPFFNDFLGLIGAVSFWPLTVYFPVQMYMSQAKVRRFSPTWTWMNVLSLACLVVSLLAAAGSIQGLIKSVAHYKPFSVSS

SEQ ID NO: 14 - DNA, 1464 bp, Oryza sativa Japonica Group
  XP_015647443 AAP6
atggggatggagaggccgcaagagaaggtggccaccaccaccaccgccgccttcaacctcgccgagtccggctacgccgaccgccccgacctcgacgacgacggccgcgagaagcgcacagggacgctggtgacggcgagcgcgcacataataacggcggtgatcggctccggcgtgctgtcgctggcgtgggcgatagcgcagctggggtgggtgatcgggccggccgtgctggtggcgttctcggtcataacctggttctgctccagcctcctcgccgactgctaccgatctcccgaccccgtccatggcaagcgcaactacacctacggccaagccgtcagggccaacctaggtgtggccaagtacaggctctgctcggtggcacagtacgtcaatctcgtcggcgtcaccattggctacaccatcactacggccatcagcatgggtgcgatcaaacggtccaactggttccatcgcaacggccacgacgcagcctgcttggcatctgacacgaccaacatgatcatatttgctggcatccaaatcctcctctcgcagctgccgaattttcacaaaatttggtggctctccattgtcgctgctgtcatgtcactggcctactcaaccattggccttggcctctccattgcaaaaattgcaggtggggcccaccccgaggcaaccctcacaggggtgactgttggagtggatgtgtctgcaagtgagaaaatctggagaacttttcagtcacttggtgacattgcctttgcatactcctactccaatgtcctcatagaaattcaggacacgctgcggtcgagcccggcggagaacgaggtgatgaagaaggcgtcgttcatcggagtctcgacgacgacgacgttctacatgctgtgcggcgtgctcggctacgcggcgttcggcaaccgcgcgccggggaacttcctcaccggcttcggcttctacgagcccttctggctcgtcgacgtcggcaacgtctgcatcgtcgtccacctcgtcggcgcctaccaggtcttctgccagcccatctaccagttcgccgaggcctgggcgcgctcgcggtggccggacagcgccttcgtcaacggcgagcgcgtgctccggctgccgctcggcgccggcgacttccccgtcagcgcgctccgcctcgtctggcgcacggcctacgtcgtgctcaccgccgtcgccgccatggcgttccccttcttcaacgacttcctcggcctcatcggcgccgtctccttctggccgctcaccgtctacttccccgtccagatgtacatgtctcaggccaaggtccggcgattctcgccgacgtggacgtggatgaacgtgctcagcctcgcctgcctcgtcgtctccctcctcgccgccgccggctccatccagggcctcatcaaatccgtcgcacattacaagccattcagcgtctcctcatga

SEQ ID NO: 15 - protein, 460 aa, Oryza sativa Japonica Group
  BAC82953.1 putative amino acid permease
MAAAGRTLGCIYAGTLVTASAHIITAVIGSGVLSLAWAIAQLGWVIGPAVLVAFSVITWFCSSLLADCYRSPDPVHGKRNYTYGQAVRANLGVAKYRLCSVAQYVNLVGVTIGYTITTAISMGAIKRSNWFHRNGHDAACLASDTTNMIIFAGIQILLSQLPNFHKIWWLSIVAAVMSLAYSTIGLGLSIAKIAGGAHPEATLTGVTVGVDVSASEKIWRTFQSLGDIAFAYSYSNVLIEIQDTLRSSPAENEVMKKASFIGVSTTTTFYMLCGVLGYAAFGNRAPGNFLTGFGFYEPFWLVDVGNVCIVVHLVGAYQVFCQPIYQFAEAWARSRWPDSAFVNGERVLRLPLGAGDFPVSALRLVWRTAYVVLTAVAAMAFPFFNDFLGLIGAVSFWPLTVYFPVQMYMSQAKVRRFSPTWTWMNVLSLACLVVSLLAAAGSIQGLIKSVAHYKPFSVSS

SEQ ID NO: 16 - DNA, 1383 bp, Oryza sativa Japonica Group
  BAC82953.1 putative amino acid permease
atggcggcggccggacgaacacttggatgcatatatgcagggacgctggtgacggcgagcgcgcacataataacggcggtgatcggctccggcgtgctgtcgctggcgtgggcgatagcgcagctggggtgggtgatcgggccggccgtgctggtggcgttctcggtcataacctggttctgctccagcctcctcgccgactgctaccgatctcccgaccccgtccatggcaagcgcaactacacctacggccaagccgtcagggccaacctaggtgtggccaagtacaggctctgctcggtggcacagtacgtcaatctcgtcggcgtcaccattggctacaccatcactacggccatcagcatgggtgcgatcaaacggtccaactggttccatcgcaacggccacgacgcagcctgcttggcatctgacacgaccaacatgatcatatttgctggcatccaaatcctcctctcgcagctgccgaattttcacaaaatttggtggctctccattgtcgctgctgtcatgtcactggcctactcaaccattggccttggcctctccattgcaaaaattgcaggtggggcccaccccgaggcaaccctcacaggggtgactgttggagtggatgtgtctgcaagtgagaaaatctggagaacttttcagtcacttggtgacattgcctttgcatactcctactccaatgtcctcatagaaattcaggacacgctgcggtcgagcccggcggagaacgaggtgatgaagaaggcgtcgttcatcggagtctcgacgacgacgacgttctacatgctgtgcggcgtgctcggctacgcggcgttcggcaaccgcgcgccggggaacttcctcaccggcttcggcttctacgagcccttctggctcgtcgacgtcggcaacgtctgcatcgtcgtccacctcgtcggcgcctaccaggtcttctgccagcccatctaccagttcgccgaggcctgggcgcgctcgcggtggccggacagcgccttcgtcaacggcgagcgcgtgctccggctgccgctcggcgccggcgacttccccgtcagcgcgctccgcctcgtctggcgcacggcctacgtcgtgctcaccgccgtcgccgccatggcgttccccttcttcaacgacttcctcggcctcatcggcgccgtctccttctggccgctcaccgtctacttccccgtccagatgtacatgtctcaggccaaggtccggcgattctcgccgacgtggacgtggatgaacgtgctcagcctcgcctgcctcgtcgtctccctcctcgccgccgccggctccatccagggcctcatcaaatccgtcgcacattacaagccattcagcgtctcctcatga

SEQ ID NO: 17 - protein, 487 aa, Oryza sativa Japonica Group
  XP_015644123.1 amino acid permease 3
MAKDVEMAVRNGDGGGGGGYYATHPHGGAGGEDVDDDGKQRRTGNVWTASAHIITAVIGSGVLSLAWATAQLGWVVGPVTLMLFALITYYTSGLLADCYRTGDPVSGKRNYTYMDAVAAYLGGWQVWSCGVFQYVNLVGTAIGYTITASISAAAVHKANCYHKNGHDADCGVYDTTYMIVFGVVQIFFSMLPNFSDLSWLSILAAVMSFSYSTIAVGLSLARTISGATGKTTLTGVEVGVDVTSAQKIWLAFQALGDIAFAYSYSMILIEIQDTVKSPPAENKTMKKATLLGVSTTTAFYMLCGCLGYAAFGNAAPGNMLTGFGFYEPYWLIDFANVCIVVHLVGAYQVFCQPIFAAVETFAARRWPGSEFITRERPVVAGRSFSVNMFRLTWRTAFVVVSTVLAIVMPFFNDILGFLGAVGFWPLTVYYPVEMYIRQRRIQRYTSRWVALQTLSLLCFLVSLASAVASIEGVSESLKHYVPFKTKS

SEQ ID NO: 18 - DNA, 1464 bp, Oryza sativa Japonica Group
  XP_015644123.1 amino acid permease 3
atggcgaaggacgtggagatggcggtgcggaacggagacggcggcggcggcggcggctactacgccacccacccgcacggcggcgccggcggcgaggacgtcgacgacgacggcaagcagcggcgaaccggtaacgtatggacggcgagcgcgcacatcatcacggcggtgatcggctccggcgtgctctctctcgcatgggcaacggcgcagctcggctgggtggtcgggccggtgactctgatgctcttcgccctcatcacgtactacacctctgggctcctcgccgactgctaccgcactggcgatccggtcagcggcaagcgcaactacacctacatggatgccgttgcggcctacttaggtggctggcaagtctggtcctgtggtgttttccaatatgtcaacctggttgggacagcaattgggtacacaatcacagcatccatcagcgcagcggctgtgcacaaggccaactgctaccacaagaacggccacgatgccgattgcggtgtctacgacaccacgtacatgatcgtctttggagtcgtccagatcttcttctccatgctgcccaacttcagtgacctctcatggctttccatcctcgccgcggtcatgtcattctcatactcgaccattgccgttggcctctcgcttgcgcgaacaatatcaggtgctactggtaagactactctgactggcgttgaggttggagttgacgtcacttcagcccagaagatctggctcgcgttccaagcgctcggtgacatcgcgttcgcctactcctactccatgatccttatagaaattcaggacacggtgaagtctccaccggcggagaacaagacgatgaagaaggcaacgctgctgggggtgtcgaccacgacggcgttctacatgctgtgcgggtgcctggggtacgcggcgttcgggaacgcggcgccggggaacatgctcaccgggttcggcttctacgagccctactggctgatcgacttcgccaacgtctgcatcgtggtccacctggtcggcgcctaccaggtgttctgccagcccatcttcgccgccgtcgagacgttcgccgccaggcggtggccgggctcggagttcatcacccgggagcgccccgtcgtggccggcaggtcgttcagcgtcaacatgttcaggctgacgtggcggacggcgttcgtggtcgtcagcacggtgctcgccatcgtgatgcccttcttcaacgacatcctgggcttcctcggcgccgtcgggttctggccgctgacggtgtactacccggtggagatgtacatccggcagcggcggatacagcggtacacgtccaggtgggtggcgctgcagacgctcagcctcctctgcttcctcgtctcgctcgcctccgccgtcgcctccatcgagggcgtcagcgagtcgctcaagcactacgtccccttcaagaccaagtcgtga

SEQ ID NO: 19 - protein, 481 aa, Oryza sativa Indica Group
  EEC68963.1 hypothetical protein OsI_37697
MSDMASGQKAKQQVMKPMEVSVEAGNAGDAAWLDDDGRARRTGTFWTASAHIITVIGSGVLSLAWAIAQLGWVAGPAVMLLFAFVIYYTSTLLAECYRTGDPATGKRNYTYMDAVRANLGGAKVTFCGVIQYANLVGVAIGYTIASSISMRAIRRAGCFHHNGHGDPCRSSSNPYMILFGVVQIVFSQIPDFDQIWWLSIVAAVMSFTYSGIGLSLGIVQTISNGGIQGSLTGISIGVGVSSTQKVWRSLQAFGDIAFAYSFSNILIEIQDTIKAPPPSEAKVMKSATRLSVATTTVFYMLCGCMGYAAFGDAAPDNLLTGFGFYEPFWLLDVANVAIVVHLVGAYQVFVQPIFAFVERWASRRWPDSAFIAKELRVGPFALSLFRLTWRSAFVCLTTVVAMLLPFFGNVVGLLGAVSFWPLTVYFPVEMYIAQRGVPRGSARWVSLKTLSACCLVVSIAAAAGSIADVIDALKVYRPFSG

SEQ ID NO: 20 - DNA, 1449 bp, Oryza sativa Indica Group
  EEC68963.1 hypothetical protein OsI_37697
atgtccgacatggcgtcggggcagaaggcgaagcagcaggtgatgaagccgatggaggtgtcggtggaggccgggaacgccggggatgcggcgtggctggacgacgacgggcgggcgcggcggacgggcacgttctggacggcgagcgcgcacatcatcaccgccgtcatcggctccggcgtgctgtcgctggcgtgggcgatcgcgcagctcgggtgggtggccggccccgccgtgatgctcctcttcgccttcgtcatctactacacctccaccctcctcgccgagtgctaccgcaccggcgacccggccaccggcaagcgcaactacacctacatggacgccgtgcgcgccaacctcggcggcgccaaggtcaccttctgcggcgtcatccagtacgccaacctcgtcggcgtcgccatcggctacaccatcgcgtcgtccatcagcatgcgcgccatcaggagggccggctgcttccaccacaacggccatggtgacccgtgccgcagctccagcaacccttacatgatcctcttcggcgtcgtgcagatcgtcttctcgcagatcccggacttcgaccagatttggtggctgtccatcgtcgccgccgtcatgtccttcacctactccggcatcggcctctccctcggcatcgtccagacaatctccaatggcgggatccagggcagcctcaccggaatcagcatcggcgtcggcgtcagctcaacgcagaaggtgtggcgcagcctgcaggcattcggcgacatcgccttcgcatactccttctccaacatcctcatcgagatccaagacacgatcaaggcgccgccgccgtcggaggcgaaggtgatgaagagcgcgacgaggctgagcgtggcgacgaccacggtgttctacatgctgtgcgggtgcatgggctacgcggcgttcggcgacgcggcgcccgacaacctcctcacgggcttcggcttctacgagcccttctggctgctcgacgtcgccaacgtcgccatcgtcgtgcacctcgtcggcgcctaccaggtgttcgtccagccaatcttcgccttcgtcgagcgctgggcctcccgccggtggccggacagcgcgttcatcgccaaggagctccgcgtggggcccttcgcgctcagcctcttccgcctgacgtggcgctcggcgttcgtctgcctcaccacagtcgtcgccatgctcctccccttcttcggcaacgtggtgggtctcctcggcgccgtctccttctggccgctcaccgtctacttccccgtcgagatgtacatcgcgcagcgcggcgtgccacgtggcagcgcgaggtgggtctcgctcaagacgctcagcgcgtgctgcctcgtcgtctccatcgccgccgccgcgggctccattgctgacgtcatcgacgctctcaaggtgtacagaccgttcagcggatga

SEQ ID NO: 21 - protein, 475 aa, Oryza sativa Indica Group
  EAY82481.1 hypothetical protein OsI_37698
MASGQKVVKPMEVSVEAGNAGEAAWLDDDGRARRTGTFWTASAHIITAVIGSGVLSLAWAIAQLGWVAGPAVMLLFAFVIYYTSTLLAECYRTGDPATGKRNYTYMDAVRANLGGAKVTFCGVIQYANLVGVAIGYTIASSISMRAIRRAGCFHHNGHGDPCRSSSNPYMILFGVVQIVFSQIPDFDQIWWLSIVAAVMSFTYSGIGLSLGIVQTISNGGIQGSLTGISIGVGVSSTQKVWRSLQAFGDIAFAYSFSNILIEIQDTIKAPPPSEAKVMKSATRLSVATTTVFYMLCGCMGYAAFGDAAPDNLLTGFGFYEPFWLLDVANVAIVVHLVGAYQVFVQPIFAFVERWASRRWPDSAFIAKELRVGPFALSLFRLTWRSAFVCLTTVVAMLLPFFGNVVGLLGAVSFWPLTVYFPVEMYIAQRGVPRGSARWISLKTLSACCLVVSIAAAAGSIADVIDALKVYRPFSG

SEQ ID NO: 22 - DNA, 1428 bp, Oryza sativa Indica Group
  EAY82481.1 hypothetical protein OsI_37698
atggcgtcggggcagaaggtggtgaagccgatggaggtgtcggtggaggccgggaacgccggggaggcggcgtggctggacgacgacgggcgggcgcggcggacgggcacgttctggacggcgagcgcgcacatcatcaccgccgtcatcggctccggcgtgctgtcgctggcgtgggcgatcgcgcagctgggctgggtggccggccccgccgtgatgctcctcttcgccttcgtcatctactacacctccaccctcctcgccgagtgctaccgcaccggcgacccggccaccggcaagcgcaactacacctacatggacgccgtgcgcgccaacctcggcggcgccaaggtcaccttctgcggcgtcatccagtacgccaacctcgtcggcgtcgccatcggctacaccatcgcgtcgtccatcagcatgcgcgccatcaggagggccggctgcttccaccacaacggccatggtgacccgtgccgcagctccagcaacccttacatgatcctcttcggcgtcgtgcagatcgtcttctcccagatccctgacttcgaccagatttggtggctgtccatcgtcgccgccgtcatgtccttcacctactccggcatcggcctctccctcggcatcgtccagactatctccaatggcgggatccagggcagcctcaccggcatcagcatcggagtcggcgtcagctcgacgcagaaggtgtggcgcagcttgcaggcattcggcgacatcgccttcgcatactccttctccaacatcctcatcgagatccaagacacgatcaaggcgccgccgccgtcggaggcgaaggtgatgaagagcgcgacgaggctgagcgtggcgacgaccacggtgttctacatgctgtgcgggtgcatgggctacgcggcgttcggcgacgcggcgcccgacaacctcctcaccggcttcgggttctacgagcccttctggctgctcgacgtcgccaacgtcgccatcgtcgtgcacctcgtcggcgcctaccaggtgttcgtccagccaatcttcgccttcgtcgagcgctgggcctcccgccggtggccggacagcgcgttcatcgccaaggagctccgcgtggggcccttcgcgctcagcctcttccgcctgacgtggcgctcggcgttcgtctgcctcaccaccgtcgtcgccatgctcctccccttcttcggcaacgtcgtgggcctcctcggcgccgtctccttctggccgctcaccgtctacttccccgtcgagatgtacatcgcgcagcgtggcgtgccgcgggggagcgcgaggtggatctccctcaagacgctcagcgcgtgctgcctagtcgtctccatcgccgccgcggcgggctccattgctgacgtcatcgacgcgctcaaggtgtacagacccttcagcggatga

SEQ ID NO: 23 - protein, 459 aa, Oryza sativa Japonica Group
  BAD53557 putative amino acid carrier
MDVYLPRTQGDVDDDGKERRTGTVWTATAHIITAVIGSGVLSLAWAMAQLGWVAGPITLLLFAAITFYTCGLLSDCYRVGDPATGKRNYTYTDAVKSYLGGWHVWFCGFCQYVNMFGTGIGYTITASISAAAINKSNCYHWRGHGTDCSQNTSAYIIGFGVLQALFCQLPNFHQLWWLSIIAAVMSFSYAAIAVGLSLAQTIMDPLGRTTLTGTVVGVDVDATQKVWLTFQALGNVAFAYSYAIILIEIQDTLRSPPPENATMRRATAAGISTTTGFYLLCGCLGYSAFGNAAPGNILTGFGFYEPYWLVDVANACIVVHLVGGFQVFCQPLFAAVEGGVARRCPGLLGGGAGRASGVNVFRLVWRTAFVAVITLLAILMPFFNSILGILGSIAFWPLTVFFPVEMYIRQRQLPRFSAKWVALQSLSLVCFLVTVAACAASIQGVLDSLKTYVPFKTRS

SEQ ID NO: 24 - DNA, 1380 bp, Oryza sativa Japonica Group
  BAD53557 putative amino acid carrier
atggacgtctaccttccccggacccaaggcgacgtcgacgacgacggcaaggagaggaggacagggacggtgtggacggcgacggcgcacataatcacggcggtgatcgggtccggcgtgctgtcgctggcgtgggcgatggcgcagctggggtgggtggctggccccatcaccctcctcctcttcgccgccatcaccttctacacctgcggcctcctctccgactgctaccgcgtcggcgacccggccaccggcaagcgcaactacacctacaccgacgccgtcaagtcctacctcggtggctggcacgtctggttctgcggcttctgccagtacgtcaacatgttcggcaccggcatcggctacaccatcaccgcctccatctccgccgcggctatcaacaagtccaactgctaccactggcgcggccatggcacggactgcagccagaacacgagcgcctacatcatcggcttcggcgtcctgcaggccctcttctgccagctcccaaacttccaccagctctggtggctgtccatcatcgccgccgtcatgtccttctcgtacgccgccatcgccgtcggcttgtcgctggcgcagaccatcatggacccgctggggaggacgacgctgacgggcacggtggtcggcgtcgacgtcgacgccacgcagaaggtgtggctcacgttccaggcgctggggaacgtcgccttcgcctactcctacgccatcatcctcatcgagatccaggacacgctgcggtcgccgccgccggagaacgcgacgatgcggcgcgccacggcggcggggatctcgacgaccacggggttctacctgctgtgcggctgcctgggctactcggcgttcgggaacgcggcgccgggcaacatcctcaccggcttcggcttctacgagccatactggctggtggacgtggccaacgcctgcatcgtggtgcacctcgtcggcgggttccaggtgttctgccagccgctgttcgccgccgtggagggcggcgtggcgcggcggtgcccggggctgctcggcggcggcgcggggcgggccagcggcgtgaacgtgttccggcttgtgtggaggacggcgttcgtggcggtgatcacgctgctggccatcctgatgcccttcttcaacagcatcctgggaatcctggggagcatcgcgttctggccgctcaccgtcttcttccccgtcgagatgtacatccggcagcggcagctgccgcggttcagcgccaagtgggtggcgctgcagagcctgagcctcgtctgcttcctcgtcaccgtcgccgcctgcgccgcctccatccagggcgtcctcgactcgctcaagacctacgtgcccttcaagaccaggtcctga

SEQ ID NO: 25 - protein, 496 aa, Oryza sativa Japonica Group
  XP_015637472.1 AAP3 isoform X2
MGENGVVASKLCYPAAAMEVVAAELGHTAGSKLYDDDGRLKRTGTMWTASAHIITAVIGSGVLSLGWAIAQLGWVAGPAVMLLFSFVTYYTSALLADCYRSGDESTGKRNYTYMDAVNANLSGIKVQVCGFLQYANIVGVAIGYTIAASISMLAIKRANCFHVEGHGDPCNISSTPYMIIFGVAEIFFSQIPDFDQISWLSILAAVMSFTYSTIGLGLGVVQVVANGGVKGSLTGISIGVVTPMDKVWRSLQAFGDIAFAYSYSLILIEIQDTIRAPPPSESRVMRRATVVSVAVTTLFYMLCGCTGYAAFGDAAPGNLLTGFGFYEPFWLLDVANAAIVVHLVGAYQVYCQPLFAFVEKWAQQRWPKSWYITKDIDVPLSLSGGGGGGGRCYKLNLFRLTWRSAFVVATTVVSMLLPFFNDVVGFLGAVGFWPLTVYFPVEMYIVQKRIPRWSTRWVCLQLLSLACLAITVASAAGSIAGILSDLKVYKPFATTY

SEQ ID NO: 26 - DNA, 1491 bp, Oryza sativa Japonica Group
  XP_015637472.1 AAP3 isoform X2
atgggggagaacggtgtggtggcgagcaagctgtgctacccggcggcggccatggaggtggtcgccgccgagctcggccacacggccggctccaagctgtacgacgacgacggccgcctcaagcgcaccgggacgatgtggacggcgagcgcgcacatcatcacggcggtgatcggctccggcgtgctgtcgctggggtgggcgatcgcgcagctgggttgggtggccggccccgccgtcatgctgctcttctcgttcgtcacctactacacctccgcgctgctcgccgactgctaccgctccggcgacgagagcaccggcaagcgcaactacacctacatggacgccgtgaacgccaacctgagtggcatcaaggtccaggtctgcgggttcctgcagtacgccaacatcgtcggcgtcgccatcggctacaccattgccgcctccattagcatgctggcgatcaagcgggcgaactgcttccacgtcgaggggcacggcgacccgtgcaacatctcgagcacgccgtacatgatcatcttcggcgtggcggagatcttcttctcgcagatcccggacttcgaccagatctcgtggctgtccatcctcgccgccgtcatgtcgttcacctactccaccatcgggctcggcctcggcgtcgtgcaggtggtggccaacggcggcgtcaaggggagcctcaccgggatcagcatcggcgtggtgacgcccatggacaaggtgtggcggagcctgcaggcgttcggcgacatcgccttcgcctactcctactccctcatcctcatcgagatccaggacaccatccgggcgccgccgccgtcggagtcgagggtgatgcggcgcgccaccgtggtgagcgtcgccgtcaccacgctcttctacatgctctgcggctgcacggggtacgcggcgttcggcgacgccgcgccgggcaacctcctcaccgggttcggcttctacgagcccttctggctcctcgacgttgccaacgccgccatcgtcgtccacctcgtcggcgcctaccaggtctactgccagccgctgttcgccttcgtcgagaagtgggcgcagcagcggtggccgaaatcatggtacatcaccaaggatatcgacgtgccgctctccctctccggcggcggcggcggcggcggaaggtgctacaagctgaacctgttcaggctgacatggaggtcggcgttcgtggtggcgacgacggtggtgtcgatgctgctgccgttcttcaacgacgtggtggggttcctcggcgcggtggggttctggccgctcaccgtctacttcccggtggagatgtacatcgtgcagaagaggataccgaggtggagcacgcggtgggtgtgcctgcagctgctcagcctcgcctgcctcgccatcaccgtcgcctccgccgccggctccatcgccggaatcctctccgacctcaaggtctacaagccgttcgccaccacctactaa

SEQ ID NO: 27 - protein, 542 aa, Oryza sativa Japonica Group
  XP_025881587 AAP3 isoform X1
MTHHTKFNPNYISICNPASSLSLIFTSLFLNWKRVRGSRRGDFCKEMGENGVVASKLCYPAAAMEVVAAELGHTAGSKLYDDDGRLKRTGTMWTASAHIITAVIGSGVLSLGWAIAQLGWVAGPAVMLLFSFVTYYTSALLADCYRSGDESTGKRNYTYMDAVNANLSGIKVQVCGFLQYANIVGVAIGYTIAASISMLAIKRANCFHVEGHGDPCNISSTPYMIIFGVAEIFFSQIPDFDQISWLSILAAVMSFTYSTIGLGLGVVQVVANGGVKGSLTGISIGVVTPMDKVWRSLQAFGDIAFAYSYSLILIEIQDTIRAPPPSESRVMRRATVVSVAVTTLFYMLCGCTGYAAFGDAAPGNLLTGFGFYEPFWLLDVANAAIVVHLVGAYQVYCQPLFAFVEKWAQQRWPKSWYITKDIDVPLSLSGGGGGGGRCYKLNLFRLTWRSAFVVATTVVSMLLPFFNDVVGFLGAVGFWPLTVYFPVEMYIVQKRIPRWSTRWVCLQLLSLACLAITVASAAGSIAGILSDLKVYKPFATTY

SEQ ID NO: 28 - DNA, 1629 bp, Oryza sativa Japonica Group
  XP_025881587 AAP3 isoform X1
atgacacaccacaccaagttcaaccccaactatatctctatttgtaaccctgcttcttctctctctttgatcttcacttctctcttcctcaattggaagagggttaggggatcaagaagaggagacttttgcaaagagatgggggagaacggtgtggtggcgagcaagctgtgctacccggcggcggccatggaggtggtcgccgccgagctcggccacacggccggctccaagctgtacgacgacgacggccgcctcaagcgcaccgggacgatgtggacggcgagcgcgcacatcatcacggcggtgatcggctccggcgtgctgtcgctggggtgggcgatcgcgcagctgggttgggtggccggccccgccgtcatgctgctcttctcgttcgtcacctactacacctccgcgctgctcgccgactgctaccgctccggcgacgagagcaccggcaagcgcaactacacctacatggacgccgtgaacgccaacctgagtggcatcaaggtccaggtctgcgggttcctgcagtacgccaacatcgtcggcgtcgccatcggctacaccattgccgcctccattagcatgctggcgatcaagcgggcgaactgcttccacgtcgaggggcacggcgacccgtgcaacatctcgagcacgccgtacatgatcatcttcggcgtggcggagatcttcttctcgcagatcccggacttcgaccagatctcgtggctgtccatcctcgccgccgtcatgtcgttcacctactccaccatcgggctcggcctcggcgtcgtgcaggtggtggccaacggcggcgtcaaggggagcctcaccgggatcagcatcggcgtggtgacgcccatggacaaggtgtggcggagcctgcaggcgttcggcgacatcgccttcgcctactcctactccctcatcctcatcgagatccaggacaccatccgggcgccgccgccgtcggagtcgagggtgatgcggcgcgccaccgtggtgagcgtcgccgtcaccacgctcttctacatgctctgcggctgcacggggtacgcggcgttcggcgacgccgcgccgggcaacctcctcaccgggttcggcttctacgagcccttctggctcctcgacgttgccaacgccgccatcgtcgtccacctcgtcggcgcctaccaggtctactgccagccgctgttcgccttcgtcgagaagtgggcgcagcagcggtggccgaaatcatggtacatcaccaaggatatcgacgtgccgctctccctctccggcggcggcggcggcggcggaaggtgctacaagctgaacctgttcaggctgacatggaggtcggcgttcgtggtggcgacgacggtggtgtcgatgctgctgccgttcttcaacgacgtggtggggttcctcggcgcggtggggttctggccgctcaccgtctacttcccggtggagatgtacatcgtgcagaagaggataccgaggtggagcacgcggtgggtgtgcctgcagctgctcagcctcgcctgcctcgccatcaccgtcgcctccgccgccggctccatcgccggaatcctctccgacctcaaggtctacaagccgttcgccaccacctactaa

SEQ ID NO: 29 - protein, 488 aa, Oryza sativa Japonica Group
  XP_015629427 AAP4
MGENVVGTYYYPPSAAAMDGVELGHAAAGSKLFDDDGRPRRNGTMWTASAHIITAVIGSGVLSLGWAIAQLGWVAGPAVMVLFSLVTYYTSSLLSDCYRSGDPVTGKRNYTYMDAVNANLSGFKVKICGFLQYANIVGVAIGYTIAASISMLAIGRANCFHRKGHGDPCNVSSVPYMIVFGVAEVFFSQIPDFDQISWLSMLAAVMSFTYSVIGLSLGIVQVVANGGLKGSLTGISIGVVTPMDKVWRSLQAFGDIAFAYSYSLILIEIQDTIRAPPPSESAVMKRATVVSVAVTTVFYMLCGSMGYAAFGDDAPGNLLTGFGFYEPFWLLDIANAAIVVHLVGAYQVFCQPLFAFVEKWAAQRWPESPYITGEVELRLSPSSRRCRVNLFRSTWRTAFVVATTVVSMLLPFFNDVVGFLGALGFWPLTVYFPVEMYVVQKKVPRWSTRWVCLQMLSVGCLVISIAAAAGSIAGVMSDLKVYRPFKGY

SEQ ID NO: 30 - DNA, 1467 bp, Oryza sativa Japonica Group
  XP_015629427 AAP4
atgggggagaacgtggttggcacgtactactacccgccttcggcggccgccatggacggcgtggagctcggccacgccgccgccggctccaagctcttcgacgacgacggccgccccaggcgcaacgggacgatgtggacggcgagcgcgcacatcatcacggcggtgatcggctccggcgtgctgtcgctggggtgggccatcgcgcagctcggctgggtggccgggccggcggtcatggtgctcttctccctcgtcacctactacacctcatccctcctctccgattgctaccgctccggcgaccccgtcaccggcaagcggaactacacctacatggacgccgtgaacgccaacctgagcgggttcaaggtgaagatctgcgggttcttgcagtacgccaacatcgtcggcgtcgccatcggctacaccatcgcggcgtccatcagcatgctggcgatcgggagggccaactgcttccacaggaaggggcacggcgacccgtgcaacgtctccagcgtgccctacatgatcgtcttcggcgtcgccgaggtcttcttctcgcagatccccgacttcgatcagatctcctggctctccatgctcgccgccgtcatgtccttcacctactccgtcatcggcctcagcctcggcatcgtccaagtcgtcgcgaacggagggttgaagggaagcctgaccgggatcagcatcggcgtggtgacgccgatggacaaggtgtggaggagcctgcaggcgttcggcgacatcgcgttcgcctactcctactcgctgatcctcatcgagatccaggacaccatccgggcgccgccgccgtcggagtcggcggtgatgaagcgcgccacggtggtgagcgtggcggtgaccacggtgttctacatgctctgcggcagcatggggtacgcggcgttcggcgacgacgcgccggggaacctcctcaccgggttcggcttctacgagcccttctggctcctcgacatcgccaacgccgccatcgtcgtccacctcgtcggcgcctaccaggtgttctgccagccgctcttcgccttcgtcgagaagtgggcggcgcagcggtggccggagtcgccgtacatcaccggggaggtggagctccgcctctcgccgtcgtcgaggcggtgcagggtgaacctgttccggtcgacgtggcgcacggcgttcgtcgtcgccaccacggtggtgtccatgctgctgcccttcttcaacgacgtggtcggcttcctcggcgcgctcggattctggccgctcaccgtctacttccccgtggagatgtacgtggtgcagaagaaggtgccacggtggagcacacggtgggtgtgcctgcagatgctcagcgtcggctgcctcgtcatctccatcgccgccgccgcgggctccatcgccggcgtcatgtcggatctcaaggtttaccgcccgttcaagggttactga

SEQ ID NO: 31 - protein, 469 aa, Glycine soja
  KHN37208 AAP6
MFVETPEDGGKNFDDDGRVKRTGTWITASAHIITAVIGSGVLSLAWAIAQMGWVAGPAVLFAFSFITYFTSTLLADCYRSPDPVHGKRNYTYSDVVRSVLGGRKFQLCGLAQYINLVGVTIGYTITASISMVAVKRSNCFHKHGHHDKCYTSNNPFMILFACIQIVLSQIPNFHKLWWLSIVAAVMSFAYSSIGLGLSVAKVAGGGEPVRTTLTGVQVGVDVTGSEKVWRTFQAIGDIAFAYAYSNVLIEIQDTLKSSPPENKVMKRASLIGILTTTLFYVLCGCLGYAAFGNDAPGNFLTGFGFYEPFWLIDFANICIAVHLVGAYQVFCQPIFGFVENWGKERWPNSHFVNGEHALKFPLFGTFPVNFFRVVWRTTYVIITALIAMMFPFFNDFLGLIGSLSFWPLTVYFPIEMYIKQSKMQKFSFTWTWLKILSWACLIVSIISAAGSIQGLAQDLKKYQPFKAQQ

SEQ ID NO: 32 - DNA, 1410 bp, Glycine soja
  KHN37208 AAP6
atgttcgtagaaacccctgaagatggtggcaaaaacttcgacgatgatggacgagtcaaaagaactggtacatggataactgcgagtgcccatatcataacggcagtgataggttctggagtgttgtcacttgcatgggcaattgcacaaatgggttgggtggcaggccctgcggttctctttgccttctctttcatcacatacttcacctccactcttcttgccgactgttatcgttcacctgaccctgttcatggcaagcgaaactacacctattcagatgttgtcagatccgtgttaggaggtaggaaatttcagctgtgtggattagctcagtacataaatcttgtcggtgtaactatcggttacacgataacggcttcaattagtatggtggcggtgaagaggtccaactgttttcacaaacatggtcatcatgataagtgctacacgtcaaacaaccctttcatgatcctctttgcctgcattcaaatcgtgcttagtcaaataccaaatttccataagctttggtggctctccattgttgcagcagttatgtcttttgcttattcttccattggccttgggctctccgtagctaaagtggcaggtggtggagaacctgtacggacaaccttaacgggggtgcaagttggggtggacgttacgggatccgagaaggtctggaggacgtttcaagctattggtgatattgccttcgcttacgcttattctaacgtgctcattgagatacaggataccctgaaatcgagccctccagaaaacaaggtcatgaaaagagcaagtttgattggcatcttgactacaaccttgttctatgtgctatgtggctgcctaggttatgcagcatttggaaacgacgcaccaggaaatttcctcacagggttcggtttctacgagcccttttggctaatagactttgctaacatctgcatagccgtacacttggttggagcatatcaggtcttctgtcagcccatatttgggttcgtagagaactggggtaaggaaaggtggcccaatagccattttgtaaatggagaacacgctttaaagtttccactatttggaaccttccctgtgaactttttcagggtggtatggagaacaacatatgtcatcatcactgctttgatagctatgatgtttccattcttcaatgacttcctaggcctgattggttcactgtcattttggccattaacggtttacttccccatagagatgtacattaagcagtcaaagatgcaaaagttttccttcacttggacatggctcaagatattgagctgggcttgcttgatcgtttctattatctcagctgctggctccatccaaggcctcgctcaagatctcaagaaatatcagcccttcaaagcccagcaataa

SEQ ID NO: 33 - protein, 479 aa, Glycine max
  XP_003526513 AAP6
MNPDQFQKNSMFVETPEDGGKNFDDDGRVKRTGTWITASAHIITAVIGSGVLSLAWAIAQMGWVAGPAVLFAFSFITYFTSTLLADCYRSPDPVHGKRNYTYSDVVRSVLGGRKFQLCGLAQYINLVGVTIGYTITASISMVAVKRSNCFHKHGHHDKCYTSNNPFMILFACIQIVLSQIPNFHKLWWLSIVAAVMSFAYSSIGLGLSVAKVAGGGEPVRTTLTGVQVGVDVTGSEKVWRTFQAIGDIAFAYAYSNVLIEIQDTLKSSPPENKVMKRASLIGILTTTLFYVLCGCLGYAAFGNDAPGNFLTGFGFYEPFWLIDFANICIAVHLVGAYQVFCQPIFGFVENWGKERWPNSHFVNGEHALKFPLFGTFPVNFFRVVWRTTYVIITALIAMMFPFFNDFLGLIGSLSFWPLTVYFPIEMYIKQSKMQKFSFTWTWLKILSWACLIVSIISAAGSIQGLAQDLKKYQPFKAQQ

SEQ ID NO: 34 - DNA, 1440 bp, Glycine max
  XP_003526513 AAP6
atgaatcctgatcagtttcagaagaacagcatgttcgtagaaacccctgaagatggtggcaaaaacttcgacgatgatggacgagtcaaaagaactggtacatggataactgcgagtgcccatatcataacggcagtgataggttctggagtgttgtcacttgcatgggcaattgcacaaatgggttgggtggcaggccctgcggttctctttgccttctctttcatcacatacttcacctccactcttcttgccgactgttatcgttcacctgaccctgttcatggcaagcgaaactacacctattcagatgttgtcagatccgtgttaggaggtaggaaatttcagctgtgtggattagctcagtacataaatcttgtcggtgtaactatcggttacacgataacggcttcaattagtatggtggcggtgaagaggtccaactgttttcacaaacatggtcatcatgataagtgctacacgtcaaacaaccctttcatgatcctctttgcctgcattcaaatcgtgcttagtcaaataccaaatttccataagctttggtggctctccattgttgcagcagttatgtcttttgcttattcttccattggccttgggctctccgtagctaaagtggcaggtggtggagaacctgtacggacaaccttaacgggggtgcaagttggggtggacgttacgggatccgagaaggtctggaggacgtttcaagctattggtgatattgccttcgcttacgcttattctaacgtgctcattgagatacaggataccctgaaatcgagccctccagaaaacaaggtcatgaaaagagcaagtttgattggcatcttgactacaaccttgttctatgtgctatgtggctgcctaggttatgcagcatttggaaacgacgcaccaggaaatttcctcacagggttcggtttctacgagcccttttggctaatagactttgctaacatctgcatagccgtacacttggttggagcatatcaggtcttctgtcagcccatatttgggttcgtagagaactggggtaaggaaaggtggcccaatagccattttgtaaatggagaacacgctttaaagtttccactatttggaaccttccctgtgaactttttcagggtggtatggagaacaacatatgtcatcatcactgctttgatagctatgatgtttccattcttcaatgacttcctaggcctgattggttcactgtcattttggccattaacggtttacttccccatagagatgtacattaagcagtcaaagatgcaaaagttttccttcacttggacatggctcaagatattgagctgggcttgcttgatcgtttctattatctcagctgctggctccatccaaggcctcgctcaagatctcaagaaatatcagcccttcaaagcccagcaataa

SEQ ID NO: 35 - protein, 479 aa, Glycine max
  NP_001242816 LOC100777963
MNSDQFQKNSMFVETPEDGGKNFDDDGRVRRTGTWITASAHIITAVIGSGVLSLAWAIAQMGWVAGPAVLFAFSFITYFTSTLLADCYRSPDPVHGKRNYTYSDVVRSVLGGRKFQLCGLAQYINLVGVTIGYTITASISMVAVKRSNCFHKHGHHVKCYTSNNPFMILFACIQIVLSQIPNFHKLWWLSIVAAVMSFAYSSIGLGLSVAKVAGGGEPVRTTLTGVQVGVDVTGSEKVWRTFQAIGDIAFAYAYSNVLIEIQDTLKSSPPENKVMKRASLIGILTTTLFYVLCGCLGYAAFGNDAPGNFLTGFGFYEPFWLIDFANICIAVHLVGAYQVFCQPIFGFVENWGRERWPNSQFVNGEHALNFPLCGTFPVNFFRVVWRTTYVIITALIAMMFPFFNDFLGLIGSLSFWPLTVYFPIEMYIKQSKMQRFSFTWTWLKILSWACLIVSIISAAGSIQGLAQDLKKYQPFKAQQ

SEQ ID NO: 36 - DNA, 1440 bp, Glycine max
  NP_001242816 LOC100777963
atgaattctgatcagtttcagaagaacagcatgttcgtagaaacccctgaagatggtggcaaaaacttcgacgatgatggacgagtcagaagaacgggtacatggataactgcgagtgcccatatcataacggcagtgatagggtcaggagtgttgtcacttgcatgggcaattgcacaaatgggttgggtggctggccctgccgttctctttgccttctctttcatcacttacttcacttccactcttcttgccgactgttatcgttcacctgatcctgttcatggcaagcgaaactacacctattccgatgttgtcagatccgtcttaggagggaggaaatttcagctgtgtggattagctcagtacataaatcttgtcggtgtaactatcggttacacgataacggcttcaattagtatggtggcggtgaagaggtcgaattgttttcacaaacatggtcatcatgttaagtgctatacgtcaaacaaccctttcatgatcctctttgcctgcattcaaatcgtgcttagccagataccaaatttccataagctctggtggctctccattgttgcagcagttatgtcttttgcttattcttccattggcctcgggctctcagtagctaaagtggcaggtggtggagagcctgtacggacaaccttaacgggggtgcaagttggggtagacgttacaggatccgagaaggtctggaggacgtttcaagctattggtgacattgccttcgcttatgcttattctaacgtgctcatcgagatacaggataccctgaaatcgagccctccagagaacaaggtcatgaaaagagcaagtttgattggcatcttgactacaaccttgttctatgtgctatgtggctgcctaggttatgcagcatttggaaacgatgcaccaggaaatttccttacagggttcggcttctacgagcccttttggctcatagactttgctaacatctgcatagccgtgcacttggttggagcatatcaggtcttctgtcagcccatatttgggttcgtagagaactggggtagggaaaggtggccaaatagccaatttgtaaatggagaacacgctttgaactttccactatgtggaaccttccctgtgaacttcttcagggtggtgtggagaacaacatatgtcatcatcactgctttgatagctatgatgtttccattcttcaatgacttcctaggcctgattggttcactgtccttttggccattaaccgtttacttccccatagaaatgtacattaagcagtcaaagatgcaaaggttttccttcacgtggacgtggctcaagatactgagctgggcttgcttgatcgtttctattatctcagctgctggttccatccaaggcctcgctcaagatctcaagaaatatcagcccttcaaagcccagcaataa

SEQ ID NO: 37 - protein, 479 aa, Glycine soja
  XP_028228300 AAP6-like
MNSDQFQKNSMFVETPEDGGKNFDDDGRVRRTGTWITASAHIITAVIGSGVLSLAWAIAQMGWVAGPAVLFAFSFITYFTSTLLADCYRSPDPVHGKRNYTYSDVVRSVLGGRKFQLCGLAQYINLVGVTIGYTITASISMVAVKRSNCFHKHGHHVKCYTSNNPFMILFACIQIVLSQIPNFHKLWWLSIVAAVMSFAYSSIGLGLSVAKVAGGGEPVRTTLTGVQVGVDVTGSEKVWRTFQAIGDIAFAYAYSNVLIEIQDTLKSSPPENKVMKRASLIGILTTTLFYVLCGCLGYAAFGNDAPGNFLTGFGFYEPFWLIDFANICIAVHLVGAYQVFCQPIFGFVENWGKERWPNSQFVNGEHALNFPLCGTFPVNFFRVVWRTTYVIITALIAMMFPFFNDFLGLIGSLSFWPLTVYFPIEMYIKQSKMQRFSFTWTWLKILSWACLIVSIISAAGSIQGLAQDLKKYQPFKAQQ

SEQ ID NO: 38 - DNA, 1440 bp, Glycine soja
  XP_028228300 AAP6-like
atgaattctgatcagtttcagaagaacagcatgttcgtagaaacccctgaagatggtggcaaaaacttcgacgatgatggacgagtcagaagaactggtacatggataactgcgagtgcccatatcataacggcagtgatagggtcaggagtgttgtcacttgcatgggcaattgcacaaatgggttgggtggctggccctgccgttctctttgccttctctttcatcacttacttcacttccactcttcttgccgactgttatcgttcacctgatcctgttcatggcaagcgaaactacacctattccgatgttgtcagatccgtcttaggagggaggaaatttcagctgtgtggattagctcagtacataaatcttgtcggtgtaactatcggttacacgataacggcttcaattagtatggtggcggtgaagaggtcgaattgttttcacaaacatggtcatcatgttaagtgctatacgtcaaacaaccctttcatgatcctctttgcctgcattcaaatcgtgcttagccagataccaaatttccataagctctggtggctctccattgttgcagcagttatgtcttttgcttattcttccattggcctcgggctctcagtagctaaagtggcaggtggtggagagcctgtacggacaaccttaacgggggtgcaagttggggtagacgttacaggatccgagaaggtctggaggacgtttcaagctattggtgacattgccttcgcttatgcttattctaacgtgctcatcgagatacaggataccctgaaatcgagccctccagagaacaaggtcatgaaaagagcaagtttgattggcatcttgactacaaccttgttctatgtgctatgtggctgcctaggttatgcagcatttggaaacgatgcaccaggaaatttcctcacagggttcggcttctacgagcccttttggctcatagactttgctaacatctgcatagccgtgcacttggttggagcatatcaggtcttctgtcagcccatatttgggttcgtagagaactggggtaaggaaaggtggcccaatagccaatttgtaaatggagaacacgctttgaactttccactatgtggaaccttccctgtgaacttcttcagggtggtgtggagaacaacatatgtcatcatcactgctttgatagctatgatgtttccattcttcaatgacttcctaggcctgattggttcactgtccttttggccattaaccgtttacttccccatagaaatgtacattaagcagtcaaagatgcaaaggttttccttcacgtggacatggctcaagatactgagctgggcttgcttgatcgtttctattatctcagctgctggttccatccaaggcctcgctcaagatctcaagaaatatcaacccttcaaagcccagcaataa

SEQ ID NO: 39 - protein, 329 aa, Glycine max
  KRH35636.1 hypothetical protein GLYMA_10G255300
MAVIRSNCFHKYGHEAKCHTSNYPYMTIFAVIQILLSQIPDFQELSGLSIIAAVMSFGYSSIGIGLSIAKIAGGNDAKTSLTGLIVGEDVTSQEKLWNTFQAIGNIAFAYAFSQVLVEIQDTLKSSPPENQAMKKATLAGCSITSLFYMLCGLLGYAAFGNKAPGNFLTGFGFYEPYWLVDIGNVFVFVHLVGAYQVFTQPVFQLVETWVAKRWPESNFMGKEYRVGKFRFNGFRMIWRTVYVIFTAVVAMILPFFNSIVGLLGAISFFPLTVYFPTEMYLVQAKVPKFSLVWIGVKILSGFCLIVTLVAAAGSIQGIIADLKIYEPFK

SEQ ID NO: 40 - DNA, 990 bp, Glycine max
  KRH35636.1 hypothetical protein GLYMA_10G255300
atggctgtcataagatcgaattgctttcacaagtatgggcacgaagcgaagtgtcatacatcaaattacccatatatgaccatctttgcggtcatacagattttattaagccaaatccctgatttccaggaactctcaggcctctctattattgctgccgtcatgtcttttggttattcttccataggcattggtctctccatagccaaaattgcaggaggaaacgatgccaagacaagtctaacggggctcatcgttggagaagacgtgacaagccaggagaaactatggaacactttccaagcaattggaaacattgcttttgcatacgccttcagtcaagtacttgttgagatacaggacacgttaaaatcaagcccaccagaaaatcaagccatgaaaaaggcaacccttgctggatgctcgatcacctcactgttttatatgttatgtggcctattaggctatgcagcattcgggaacaaggcacccggaaacttcttaacaggatttgggttttatgaaccatattggcttgttgacattggtaatgtcttcgtatttgttcatttagtgggcgcctaccaggtattcacacaaccagttttccagcttgtggaaacttgggttgcgaagcgttggcctgaaagcaacttcatgggaaaagaatatcgtgttggcaagttcagattcaatggattcaggatgatatggaggacagtgtacgtgattttcacagcagtggttgctatgatacttcccttcttcaacagcattgtgggtttgcttggagctatttccttctttcctttgacagtgtattttccaacagagatgtatctggtgcaggctaaagtgcccaagttttctctggtctggattggggtcaaaattctaagtggcttctgcttgattgtcactcttgttgctgcagctggatcaatccaaggaatcatcgcagaccttaaaatctatgagcccttcaagtaa

SEQ ID NO: 41 - protein, 513 aa, Glycine soja
  XP_028192809.1 AAP4-like
MLPRSRTLPSRIHQGIIEERHDVRPYVQVEVRPNNIQTETQAMNIQSNYSKCFDDDGRLKRTGTFWTATAHIITAVIGSGVLSLAWAVAQLGWVAGPVVMFLFAVVNLYTSNLLTQCYRTGDSVNGHRNYTYMEAVKSILGGKKVKLCGLIQYINLFGVAIGYTIAASVSMMAIKRSNCYHSSHGKDPCHMSSNGYMITFGIAEVIFSQIPDFDQVWWLSIVAAIMSFTYSSVGLSLGVAKVAENKTFKGSLMGISIGTVTQAGTVTSTQKIWRSLQALGAMAFAYSFSIILIEIQDTIKSPPAEHKTMRKATTLSIAVTTVFYLLCGCMGYAAFGDNAPGNLLTGFGFYNPYWLLDIANLAIVIHLVGAYQVFSQPLFAFVEKWSVRKWPKSNFVTAEYDIPIPCFGVYQLNFFRLVWRTIFVLLTTLIAMLMPFFNDVVGILGAFGFWPLTVYFPIDMYISQKKIGRWTSRWIGLQLLSVSCLIISLLAAVGSMAGVVLDLKTYKPFKTSY

SEQ ID NO: 42 - DNA, 1542 bp, Glycine soja
  XP_028192809.1 AAP4-like
atgttgccaagaagtagaacccttcctagcagaatccaccaaggaattatagaagagaggcacgatgtcaggccctacgtacaagtagaagtgcgacccaataatatccaaacggagacccaagcgatgaatatccagtctaactattccaagtgcttcgatgatgatggtcgcttgaagagaacaggaacattttggacggcaactgctcatatcatcactgctgtgatagggtcgggagtcctttcactagcatgggcggttgctcagcttggttgggttgctggacctgttgtcatgtttctctttgccgtcgtcaatctctacacttccaacctattaacacagtgttacaggaccggtgactccgttaatgggcacagaaattacacctacatggaggctgtcaagtccatcttgggaggaaaaaaggtcaagttatgtggcctcatccaatatatcaatctgtttggagttgcaatcgggtacaccattgctgcctctgtcagtatgatggccataaaaaggtcgaattgctatcacagcagtcatggaaaagatccctgccacatgtcaagcaatgggtatatgataacatttggaatagcagaagtgatattttcccaaatcccagactttgatcaggtgtggtggctatccatagttgcagctatcatgtccttcacttattcttcagttggattgagtcttggagtggccaaagtagcagaaaataaaactttcaaaggaagcctgatgggaattagcattggcacagtaacacaagccggaacagtcaccagcacacagaaaatatggaggagtttacaagctcttggggcaatggcctttgcatactccttttccattatcctcatcgaaattcaggacaccataaaatctcctcctgcagagcacaagaccatgagaaaggccacaacattgagcatcgcggttaccacagtgttctatttactctgtggatgcatgggttatgcagccttcggagataatgcacctggaaatctcttgactggttttgggttctataacccttattggcttctggacattgccaaccttgcaattgttatccacctagttggggcatatcaggttttttcccagcccttatttgcatttgtggaaaaatggagtgtacgcaaatggccaaagagcaattttgtcacggcagaatatgatataccgattccctgctttggtgtgtaccaactcaacttcttccgcttagtatggagaaccatttttgtgctgttgacgaccctcatagccatgctcatgccttttttcaacgatgtggttggaatacttggcgcttttgggttctggcccttaacagtttatttccctatcgacatgtatatttcgcaaaagaagattggacgatggactagtcggtggattggacttcaattacttagtgtcagttgcctcatcatttcattgttagctgcagttggttccatggcaggggttgttttggacctcaagacttataagccatttaaaactagttattaa

SEQ ID NO: 43 - protein, 513 aa, Glycine max
  XP_006590854.1; XP_003540867; AAP4
MLPRSRTLPSRIHQGIIEERHNVRHYLQVEVRPNNTQTETEAMNIQSNYSKCFDDDGRLKRTGTFWMATAHIITAVIGSGVLSLAWAVAQLGWVAGPIVMFLFAVVNLYTSNLLTQCYRTGDSVTGHRNYTYMEAVNSILGGKKVKLCGLIQYINLFGVAIGYTIAASVSMMAIKRSNCYHSSHGKDPCHMSSNGYMITFGIAEVIFSQIPDFDQVWWLSIVAAIMSFTYSSVGLSLGVAKVAENKSFKGSLMGISIGTVTQAGTVTSTQKIWRSLQALGAMAFAYSFSIILIEIQDTIKSPPAEHKTMRKATTLSIAVTTVFYLLCGCMGYAAFGDNAPGNLLTGFGFYNPYWLLDIANLAIVIHLVGAYQVFSQPLFAFVEKWSARKWPKSNFVTAEYDIPIPCFGVYQLNFFRLVWRTIFVLLTTLIAMLMPFFNDVVGILGAFGFWPLTVYFPIDMYISQKKIGRWTSRWLGLQLLSASCLIISLLAAVGSMAGVVLDLKTYKPFKTSY

SEQ ID NO: 44 - DNA, 1542 bp, Glycine max
  XP_006590854.1; XP_003540867; AAP4
atgttgccaagaagtagaacccttcctagcagaatccaccaaggaattatagaagagaggcataatgtcaggcactacttacaagttgaagtgcgacccaataatacccaaacggagaccgaagcgatgaatatccagtctaactattccaagtgcttcgatgatgatggtcgcttgaagagaacaggaacattttggatggcaactgctcatatcatcactgctgtgataggctcaggagtcctttcactagcatgggcggttgctcagcttggttgggttgctggacctattgtcatgtttctctttgccgtcgtcaatctctacacttccaacctattaacacagtgttacaggaccggtgactccgttactggacacagaaattacacctacatggaggcagtcaactccatcttgggaggaaaaaaggtcaagttatgtggcctcatccaatatatcaatctgtttggagttgcaattggatacaccattgctgcctctgtcagtatgatggccataaaaaggtcgaattgttatcacagcagtcatggaaaagatccctgccacatgtcaagcaatgggtatatgataacattcggaatagcagaagtgatattttcccaaatcccagactttgatcaggtgtggtggctatccatagttgcagctatcatgtccttcacttattcttcagttggattgagtcttggcgtggccaaagtagcagaaaataaaagtttcaaaggaagcctgatgggaattagcattggcacagtaacacaagccggaacagtcactagcacacagaaaatatggaggagtttacaagctctcggggcaatggcctttgcatactccttttccattatcctcatcgaaattcaggacaccataaaatctcctcctgcagagcacaagaccatgagaaaggccacaactttgagcatcgcagttactacagtgttctatttactctgtggatgcatgggttatgcagccttcggagataatgcacctggaaacctcttgactggttttgggttctataacccttactggcttctggacattgccaaccttgcaattgttatccacctagttggggcataccaggttttttcccagcccttatttgcatttgtggaaaaatggagtgcacgtaaatggccaaagagcaattttgtcaccgcagaatatgatatacccattccctgctttggtgtgtaccaactcaacttcttccgcttagtatggaggaccatttttgtgctgttgacgaccctcatagccatgctcatgccttttttcaacgatgtggttggaatacttggcgcttttgggttctggcccttgacagtttatttccctattgacatgtatatttcgcaaaagaagattggacgatggaccagtcgctggcttggacttcagttacttagtgccagttgcctcatcatttcattgttagctgcagttggttccatggcaggggtggttttggacctcaagacttacaagccatttaaaactagttattaa

SEQ ID NO: 45 - protein, 471 aa, Glycine soja
  RZB79331.1 AAP2 isoform B
MNIQSNYSKCFDDDGRLKRTGTFWMATAHIITAVIGSGVLSLAWAVAQLGWVAGPIVMFLFAVVNLYTSNLLTQCYRTGDSVTGHRNYTYMEAVNSILGGKKVKLCGLIQYINLFGVAIGYTIAASVSMMAIKRSNCYHSSHGKDPCHMSSNGYMITFGIAEVIFSQIPDFDQVWWLSIVAAIMSFTYSSVGLSLGVAKVAENKSFKGSLMGISIGTVTQAGTVTSTQKIWRSLQALGAMAFAYSFSIILIEIQDTIKSPPAEHKTMRKATTLSIAVTTVFYLLCGCMGYAAFGDNAPGNLLTGFGFYNPYWLLDIANLAIVIHLVGAYQVFSQPLFAFVEKWSARKWPKSNFVTAEYDIPIPCFGVYQLNFFRLVWRTIFVLLTTLIAMLMPFFNDVVGILGAFGFWPLTVYFPIDMYISQKKIGRWTSRWLGLQLLSASCLIISLLAAVGSMAGVVLDLKTYKPFKTSY

SEQ ID NO: 46 - DNA, 1416 bp, Glycine soja
  RZB79331.1 AAP2 isoform B
atgaatatccagtctaactattccaagtgcttcgatgatgatggtcgcttgaagagaacaggaacattttggatggcaactgctcatatcatcactgctgtgataggctcaggagtcctttcactagcatgggcggttgctcagcttggttgggttgctggacctattgtcatgtttctctttgccgtcgtcaatctctacacttccaacctattaacacagtgttacaggaccggtgactccgttactggacacagaaattacacctacatggaggcagtcaactccatcttgggaggaaaaaaggtcaagttatgtggcctcatccaatatatcaatctgtttggagttgcaattggatacaccattgctgcctctgtcagtatgatggccataaaaaggtcgaattgttatcacagcagtcatggaaaagatccctgccacatgtcaagcaatgggtatatgataacattcggaatagcagaagtgatattttcccaaatcccagactttgatcaggtgtggtggctatccatagttgcagctatcatgtccttcacttattcttcagttggattgagtcttggcgtggccaaagtagcagaaaataaaagtttcaaaggaagcctgatgggaattagcattggcacagtaacacaagccggaacagtcactagcacacagaaaatatggaggagtttacaagctctcggggcaatggcctttgcatactccttttccattatcctcatcgaaattcaggacaccataaaatctcctcctgcagagcacaagaccatgagaaaggccacaactttgagcatcgcagttactacagtgttctatttactctgtggatgcatgggttatgcagccttcggagataatgcacctggaaacctcttgactggttttgggttctataacccttactggcttctggacattgccaaccttgcaattgttatccacctagttggggcataccaggttttttcccagcccttatttgcatttgtggaaaaatggagtgcacgtaaatggccaaagagcaattttgtcaccgcagaatatgatatacccattccctgctttggtgtgtaccaactcaacttcttccgcttagtatggaggaccatttttgtgctgttgacgaccctcatagccatgctcatgccttttttcaacgatgtggttggaatacttggcgcttttgggttctggcccttgacagtttatttccctattgacatgtatatttcgcaaaagaagattggacgatggaccagtcgctggcttggacttcagttacttagtgccagttgcctcatcatttcattgttagctgcagttggttccatggcaggggtggttttggacctcaagacttacaagccatttaaaactagttattaa

SEQ ID NO: 47 - protein, 513 aa, Glycine max
  AAK33098.1 amino acid transporter
MLPRSRTLPSRIHQGIIEERHNVRHYLQVEVRPNNTQTETEAMNIQSNYSKCFDDDGRLKRTGTFWMATAHIITAVIGSGVLSLAWAVAQLGWVAGPIVMFLFAVVNLYTSNLLTQCYRTGDSVSGHRNYTYMEAVNSILGGKKVKLCGLTQYINLFGVAIGYTIAASVSMMAIKRSNCYHSSHGKDPCHMSSNGYMITFGIAEVIFSQIPDFDQVWWLSIVAAIMSFTYSSVGLSLGVAKVAENKSFKGSLMGISIGTVTQAGTVTSTQKIWRSLQALGAMAFAYSFSIILIEIQDTIKSPPAEHKTMRKATTLSIAVTTVFYLLCGCMGYAAFGDNAPGNLLTGFGFYNPYWLLDIANLAIVIHLVGAYQVFSQPLFAFVEKWSARKWPKSNFVTAEYDIPIPCFGVYQLNFFRLVWRTIFVLLTTLIAMLMPFFNDVVGILGAFGFWPLTVYFPIDMYISQKKIGRWTSRWLGLQLLSASCLIISLLAAVGSMAGVVLDLKTYKPFKTSY

SEQ ID NO: 48 - DNA, 1542 bp, Glycine max
  AAK33098.1 amino acid transporter
atgttgccaagaagtagaacccttcctagcagaatccaccaaggaattatagaagagaggcataatgtcaggcactacttacaagttgaagtgcgacccaataatacccaaacggagaccgaagcgatgaatatccagtctaactattccaagtgcttcgatgatgatggtcgcttgaagagaacaggaacattttggatggcaactgctcatatcatcactgctgtgataggctcaggagtcctttcactagcatgggcggttgctcagcttggttgggttgctggacctattgtcatgtttctctttgccgtcgtcaatctctacacttccaacctattaacacagtgttacaggaccggtgactccgtttctggacacagaaattacacctacatggaggcagtcaactccatcttgggaggaaaaaaggtcaagttatgtggcctcacccaatatatcaatctgtttggagttgcaattggatacaccattgctgcctctgtcagtatgatggccataaaaaggtcgaattgttatcacagcagtcatggaaaagatccctgccacatgtcaagcaatgggtatatgataacattcggaatagcagaagtgatattttcccaaatcccagactttgatcaggtgtggtggctatccatagttgcagctatcatgtccttcacttattcttcagttggattgagtcttggcgtggccaaagtagcagaaaataaaagtttcaaaggaagcctgatgggaattagcattggcacagtaacacaagccggaacagtcactagcacacagaaaatatggaggagtttacaagctctcggggcaatggcctttgcatactccttttccattatcctcatcgaaattcaggacaccataaaatctcctcctgcagagcacaagaccatgagaaaggccacaactttgagcatcgcagttactacagtgttctatttactctgtggatgcatgggttatgcagccttcggagataatgcacctggaaacctcttgactggttttgggttctataacccttactggcttctggacattgccaaccttgcaattgttatccacctagttggggcataccaggttttttcccagcccttatttgcatttgtggaaaaatggagtgcacgtaaatggccaaagagcaattttgtcaccgcagaatatgatatacccattccctgctttggtgtgtaccaactcaacttcttccgcttagtatggaggaccatttttgtgctgttgacgaccctcatagccatgctcatgccttttttcaacgatgtggttggaatacttggcgcttttgggttctggcccttgacagtttatttccctattgacatgtatatttcgcaaaagaagattggacgatggaccagtcgctggcttggacttcagttacttagtgccagttgcctcatcatttcattgttagctgcagttggttccatggcaggggtggttttggacctcaagacttacaagccatttaaaactagttattaa

SEQ ID NO: 49 - protein, 479 aa, Glycine max
  XP_003542145.1; XP_006596210; XP_003522571; XP_003527948;AAP3
MMENGGKQTFEVSNDTLQRVGSKSFDDDGRLKRTGTIWTASAHIITAVIGSGVLSLAWAIAQLGWIAGPVVMILFSIVTYYTSTLLATCYRSGDQLSGKRNYTYTQAVRSYLGGFSVKFCGWVQYANLFGVAIGYTIAASISMMAIKRSNCYHSSGGKNPCKMNSNWYMISYGVSEIIFSQIPDFHELWWLSIVAAVMSFTYSFIGLGLGIGKVIGNGRIKGSLTGVTIGTVTESQKIWRTFQALGNIAFAYSYSMILIEIQDTIKSPPAESETMSKATLISVLVTTVFYMLCGCFGYASFGDASPGNLLTGFGFYNPFWLIDIANAGIVIHLVGAYQVYCQPLFSFVESNAAERFPNSDFMSREFEVPIPGCKPYKLNLFRLVWRTLFVILSTVIAMLLPFFNDIVGLIGAIGFWPLTVYLPVEMYITQTKIPKWGIKWIGLQMLSVACFVITILAAAGSIAGVIDDLKVYKPFVTSY

SEQ ID NO: 50 - DNA, 1440 bp, Glycine max
  XP_003542145.1; XP_006596210; XP_003522571; XP_003527948;AAP3
atgatggaaaacggtggcaaacagacatttgaagtctccaatgacacgcttcaacgagtaggttccaagagctttgatgatgatggccgtctcaaaagaactggaactatttggactgcaagtgcccacataataacagctgttattggttctggggtgctatctttggcttgggctattgctcagctaggttggattgctggtcctgtggtgatgattctattctctattgtgacttattatacctcaactcttctagctacttgttaccgttctggtgaccaactcagtggcaagagaaactacacttacacacaagctgttagatcctaccttggcggtttttcggtcaagttttgtgggtgggttcagtatgcgaacctttttggagtggcaattgggtacaccatagcagcttccataagcatgatggcaatcaaaaggtctaattgttatcatagtagcggggggaaaaatccatgcaaaatgaacagcaattggtacatgatttcatatggtgtttcggaaattatcttctcccaaattccagatttccatgagttgtggtggctctctattgtagctgctgtcatgtccttcacatactcattcattggacttggccttggtattggtaaagttataggaaacggaagaattaaaggaagcctaactggtgtaactattgggactgtgacagaatcccaaaaaatttggagaactttccaagcgcttggaaacatagcctttgcttactcctactcaatgatccttattgaaattcaggacacaatcaaatcccctccagcagagtcagagacaatgtccaaggctactttaataagtgttttggtcacaaccgttttctatatgctatgtggttgctttggctatgcttcttttggagatgcaagtccgggaaaccttctcactggctttggcttctataacccattttggctcattgacatagccaatgctggcattgttatccaccttgttggtgcataccaagtttactgccaacccctcttctcattcgtcgaatcaaatgcggcagaaaggttccctaatagtgattttatgagcagagagtttgaagtaccaatccctggttgcaaaccctacaagctcaacctcttcaggttggtttggaggacactttttgtgattttgtcaactgtgatagccatgctcctaccattcttcaatgacattgtagggcttattggagccattggattttggcccctcactgtgtatttaccagtggagatgtatataactcaaactaagataccaaagtggggcataaaatggataggcctacaaatgcttagtgttgcatgctttgtaattactatattagctgcagcaggttccattgctggggttattgatgatcttaaagtttacaagccatttgttaccagctactaa

SEQ ID NO: 51 - protein, 478 aa, Glycine soja
  KHN19623.1; KHN44307; AAP 3
MENGGKQTFEVSNDTLQQGGSKSFDDDGRLKRTGTIWTASAHIVTAVIGSGVLSLAWAIAQLGWLAGPIVMILFSIVTYYTSTLLACCYRSGDQLSGKRNYTYTQAVRSNLGGLAVMFCGWVQYANLFGVAIGYTIAASISMMAVKRSNCYHSSGGKNPCKMNSNWYMISYGVAEIIFSQIPDFHELWWLSIVAAVMSFTYSFIGLGLGIGKVIGNGRIKGSLTGVTVGTVTESQKIWRSFQALGNIAFAYSYSMILIEIQDTIKSPPAESQTMSKATLISVLITTVFYMLCGCFGYASFGDASPGNLLTGFGFYNPYWLIDIANVGIVIHLVGAYQVYCQPLFSFVESHAAARFPNSDFMSREFEVPIPGCKPYRLNLFRLVWRTIFVILSTVIAMLLPFFNDIVGLIGAIGFWPLTVYLPVEMYITQTKIPKWGPRWICLQMLSAACFVVTLLAAAGSIAGVIDDLKVYKPFVTSY

SEQ ID NO: 52 - DNA, 1437 bp, Glycine soja
  KHN19623.1; KHN44307; AAP 3
atggaaaacggtggcaaacagacatttgaagtctcaaatgacacgcttcaacaaggaggttccaagagctttgatgatgatggccgtctcaaaagaactggaactatatggactgcaagtgcccacatagtaacagctgttattggttctggggtgctatctttggcttgggcgattgctcagctaggttggcttgctggtcctattgtgatgattctgttctctattgtgacttattatacctcaactcttctagcttgttgttaccgttctggtgaccaactcagtggcaagagaaactacacttacacacaagctgttagatccaaccttggtggtcttgcggtcatgttttgtgggtgggttcagtatgcaaacctatttggagtggcaattgggtacaccatagcagcttccataagcatgatggcagtcaaaaggtctaattgttatcatagtagcggagggaaaaatccatgcaaaatgaatagcaattggtacatgatttcatatggtgttgcggaaattatcttctcccaaattccagatttccatgagttgtggtggctctctattgtagctgctgtcatgtccttcacatactcattcattggacttggccttggtattggtaaagttataggaaacggaagaattaaaggaagcctaactggtgtaactgttgggactgtgacagaatcccagaaaatttggaggagtttccaagctcttggtaacatagcctttgcctactcctactcaatgatccttattgaaattcaggacacaatcaaatctcctccagcagagtcacagacaatgtccaaggctactttaatcagtgttttgatcacaaccgttttctatatgttatgtggctgctttggctatgcttctttcggagatgcaagcccgggaaaccttctcactggcttcggcttctataacccatattggctcattgacatagccaatgttggcatagttatccaccttgttggtgcataccaagtttactgccaacccctcttctcattcgtggaatcacatgcagcagcaaggttcccaaatagtgattttatgagcagagagtttgaagtaccaatccctggctgcaaaccctacaggctcaacctcttcaggttggtttggaggacaatttttgtgattttgtcaactgtgatagccatgctcctaccattcttcaatgacattgtagggcttattggagccattggattttggcccctcactgtttatttaccagtggagatgtatataactcaaactaagataccaaagtggggcccaagatggatatgcctacaaatgcttagtgctgcatgctttgtagttactctattagctgcagcaggttccattgctggggttattgatgatcttaaagtttacaagccattcgtcaccagctactaa

SEQ ID NO: 53 - protein, 454 aa, Glycine soja
  RZC18207.1 AAP3 isoform D
MMCLRCTGTVWTASAHIITAVIGSGVLSLAWAIAQLGWIAGPIVMVLFSAITYYTSTLLSDCYRTGDPVTGKRNYTYMDAIQSNFGGNGFKVKLCGLVQYINLFGVAIGYTIAASTSMMAIERSNCYHKSGGKDPCHMNSNMYMISFGIVEIIFSQIPGFDQLWWLSIVAAVMSFTYSTIGLGLGIGKVIENRGVGGSLTGITIGTVTQTEKVWRTMQALGDIAFAYSYSLILVEIQDTVKSPPSESKTMKKASFISVAVTSIFYMLCGCFGYAAFGDASPGNLLTGFGFYNPYWLLDIANAAIVIHLVGSYQVYCQPLFAFVEKHAARMLPDSDFVNKEIEIPIPGFHSYKVNLFRLVWRTIYVMVSTVISMLLPFFNDIGGLLGAFGFWPLTVYFPVEMYINQKRIPKWSTKWICLQILSMACLLMTIGAAAGSIAGIAIDLQTYKPFKTNY

SEQ ID NO: 54 - DNA, 1365 bp, Glycine soja
  RZC18207.1 AAP3 isoform D
atgatgtgtttgagatgtacagggacggtgtggactgcaagtgcacacataataactgcagtgattgggtctggggtgctgtctctggcttgggctatagctcagcttggatggattgctggtcctattgtcatggttctcttttctgccatcacttactacacttccactcttctctctgattgttatcgtactggtgatcctgtaactggcaagagaaactacacttacatggacgctattcagtctaactttggtggaaatggctttaaggtcaagctgtgtgggctagttcagtacattaaccttttcggagtcgccattggttacactatagcggcttccactagcatgatggcaattgaaagatctaattgttaccacaagagtggagggaaagatccatgtcatatgaacagtaacatgtacatgatttcatttggtatagtggaaattattttctcacaaattccgggcttcgatcaattgtggtggctctccattgtagctgctgtcatgtccttcacatactccactattgggctaggccttggtattggaaaagttattgaaaatagaggagtcgggggaagcctaaccgggataacaattggtaccgtgacacaaactgaaaaagtttggagaaccatgcaagctcttggtgacatagcctttgcctattcatactccctcatccttgtagaaattcaggacacagtgaaatcccctccatcagagtcaaaaacaatgaagaaggctagtttcatcagtgttgcagtaaccagcattttctacatgctttgtggttgctttggttatgctgcttttggagatgcaagccctggaaaccttctcactggctttggtttctacaacccatattggctccttgacatagctaatgctgccatagtgatccaccttgttggttcataccaagtttactgccagcccctcttcgccttcgttgagaaacacgcggcgcgtatgctcccagatagtgattttgtgaacaaagaaattgaaattccaatccctggtttccattcctacaaggtcaacctcttcagattggtttggaggacaatatatgtaatggtgagcactgtaatatcaatgctcctcccattcttcaatgacatagggggacttcttggagcatttggattttggccccttactgtgtatttcccagtggagatgtacattaatcaaaagagaataccaaaatggagcacaaagtggatctgcctccaaatacttagcatggcttgccttttgatgactataggagctgcagctggctctattgctgggattgccattgatcttcaaacttacaagcctttcaaaaccaactattga

SEQ ID NO: 55 - protein, 504 aa, Glycine soja
  RZC13226.1 Amino acid permease 2 isoform B, partial
KFALFLRVFCVWKFSFHQIKMPENAATTNLNHLQVFGIEDDVPSHSQNNSKCYDDDGRLKRTGNVWTASSHIITAVIGSGVLSLAWAIAQLGWIAGPTVMFLFSLVTFYTSSLLADCYRAGDPNSGKRNYTYMDAVRSILGGANVTLCGIFQYLNLLGIVIGYTIAASISMMAIKRSNCFHKSGGKNPCHMSSNVYMIIFGATEIFLSQIPDFDQLWWLSTVAAIMSFTYSIIGLSLGIAKVAETGTFKGGLTGISIGPVSETQKIWRTSQALGDIAFAYSYAVVLIEIQDTIKSPPSEAKTMKKATLISIAVTTTFYMLCGCMGYAAFGDAAPGNLLTGFGFYNPYWLIDIANAAIVIHLVGAYQVFSQPIFAFVEKEVTQRWPHIEREFKIPIPGFSPYKLKVFRLVLRTVFVVLTTVISMLLPFFNDIVGVIGALGFWPLTVYFPVEMYISQKKIPKWSNRWISLKIFSVACLIVSVVAAVGSVAGVLLDLKKYKPFHSHY

SEQ ID NO: 56 - DNA, 4405 bp, Glycine soja
  RZC13226.1 Amino acid permease 2 isoform B, partial
atgcctgaaaacgcagccacaaccaaccttaaccaccttcaagtttttggcatcgaagatgatgtgccttcgcattcacagaataactccaaatgctatgatgatgatggccgtctcaaacgaactggtacaaaattcattttagctgctttcacttttactaacaataatacacgactctgtttcatataaactagaatttagttagaatgaatcgacacgctgataaacaattttttattatgttatctaattataaaaattttgtacaataaaattattaaattttataataataaaatttatatttggtatatttattgaccgtgtaaactgtttatagagattttataacaactcttaagttttagtctattgactttaataataattatcttaaaatttatatttaactttatttttaaataattttttaatattatcaatgtatccataaaaatgaaactataaaatttaactttgtaatataactgttccactatttgaaaatcaaacatcagacattgttaattattgtttctatctccattttttttaaaatctttttgccccacacacatctcaaaattccttcaaaaatccaaggaaataacaaaagctgaccctttttttatgtacaaaaagtatttttcataatgtaaattaatatataatgtacatgtgatgatatgagacaggaaatgtttggactgcaagctcgcacataataaccgcagtgataggatcaggggtgctgtcattagcttgggccatagctcagctaggttggattgctggtcctactgtcatgttcttattctctttggttaccttttatacttcatccttgttggctgattgttatcgtgccggtgaccccaattctggcaagagaaactacacttacatggacgcagttcgctccattcttggtaacaattcattcattcattcattcagtcagtattgctttttttatattcttttcacaaattaatgatatgcatgcatgttgttgaccttaaatgatactatatcatatagtacatatagtatatattgttggaaagtctcacatcatattatctattttaatttttgagatgtaatttatatatttgttgagtaattttataatatcaataagatgtatgtaataaattaaacattaataacttataattaatgggattgtcatgatcatactgatgataaggtcattatcacctcatgaaaatatatgagattatgtccgccaaagaagatgctacttgtcaaaagggattcaactcatgcacatgctcttgttcacattctactagctagttatctaaaacttaatataacatgcactttagggttggcgaatttaatataactcatgcacatggtctttgtcttttattaccaaaaaaagaaagggagttttccctagaaaatacgatttaatttttaattgccaattttgtattctgtagtcttaatatatataaaattgtctggccaatgcctcgtgttggctgcacagtgaatagcaatattttttattttttctcttttattctcactttctctttttgatttttaaaaaaccctagactctagtattatgataaatctcctcactaacggtatgccacgtgcctgtcaaagaaaaatagtatgccacgcgtattaatttcccactctaacattaatcattaatatgtgcgcaaaccaagagcatcttaaattgatgatttatttgtttgttacacttacaagaacaggttacagtatccagcttgctttactgagagggttttgctaattagtgtttttagatattggttaacggagttaaaaaaatatatttattataaaaattatagacaaatataaaaaagtcataaaaaatataattttatatattttaacaaaaaaaattagtttctcaatcaatacttcaaaaatattgattaacatttaccttatttcaatacttctcattcaaccatatatatacatgggataatattaaattaaaaaaatatttattataaaaattatatataaatataaaaaaaagtcataaacaacacaattttgcatgtattagcaaaaataataatttcttaatcaatgcttcaaaatattagttaacatttaccttatttcaatacttctcattcaaccatatatatacatgggataatattaaattaatttaacttttttaactagtttggctaaatcaagcttatttaatgtctatgatagtactttggaaattggaagatgcatgtataataattttaacgtatcaataaccaaattaaaaattaggtcaaaagtttgagaaggcagaaagagcatacaaatttcatatgatttataaataatagcttagttcagataaacttattaataattttaacatatgattttatcttatatttcatattgtacaatgcaatataatgtattttgcaggtggagccaatgttacgttgtgcggaatatttcagtacctgaatctattgggaattgtaataggatacacaattgccgcttctattagcatgatgtaagttcagatttaaacttcataatgaatgatattacttgtagtctaccaacatcatttcatgtcagtgttatttcttatcatttaatgataattgagcctatctcattttccacacacttttattttgatttggatcaaaattagttacctaaaaactacacaaatacttaaaaaaaaatgttcacgagttaactatgagtaaaatatctaatgattgtatcacacacaagtgacacaacctgaaattaatttccttgaactgattgatagggatagtattatacactacagtttgatgatcattaatgggtttgattcagtttcatagattttgattgacagggcaattaaaaggtcaaactgtttccataaatctgggggcaaaaacccatgtcacatgtcaagcaacgtatacatgatcatttttggcgcaaccgaaattttcctttctcaaattcccgattttgatcaattatggtggctctcaacagttgctgcaataatgtctttcacctattccataattggtctctctcttggaattgccaaagttgcaggttattatattgaaaatgctctcctacacttttagttttttttaatacaacacttttcttagggttacattgcatcatataggcttaaattttggttggttcatgaacagaaacgggtaccttcaagggtggcctcactggaatcagcattggaccagtgtcagagacccaaaaaatctggaggacttcccaagctcttggtgatatagcctttgcctattcatatgctgtggttcttatagaaattcaggtatctccttcaaaaactaagaattagttaatctaaaaaaggttaaaatgttcatttgatttttatagttgttctcattttaaagtttcgtccatgttcagaaggaaaaaaaaaacaatagcttttagtccctacacaattttttgtggcagtttatcactactttttgtaactgtcaaaaataagttttgatattctcagcaagtaccaaaacttaacttaaacacattgtagggactaaaacttattattttttcgtataaaaactaaaacttaaaatgaagagaactgtagagatcaaatgagtagtttaacattaagaaaagaagaatatgatttattacatagtaagctagccaaaatataatattttgctgactacaggacacaataaaatctccaccgtctgaagcaaaaacaatgaagaaggccacattgataagtattgcagtgaccacaacattttacatgctctgtggctgcatggggtatgctgcttttggagatgctgcaccggggaatctgctaactggctttggcttctataacccatattggcttatagacattgcaaatgcagctatagttattcaccttgtgggagcataccaagtgttttcccaacccatctttgcctttgtggagaaagaggtaacacaaagatggccccacattgagagggagttcaagattccaattcctggtttctccccttacaaacttaaggtgtttagattagttttgaggacagtgtttgttgtcctaacaactgtcatatcaatgctgcttccattcttcaatgacattgttggagtgattggggcattgggattttggcccttaactgtttactttcctgtggagatgtatatttcacagaagaagatcccaaaatggagtaacagatggattagcctcaaaatatttagtgtggcctgcctcatagtatcagttgttgctgctgttggctcagtggcaggagtcttgcttgaccttaagaaatacaaaccattccactcacactattaa

SEQ ID NO: 57 - protein, 484 aa, Glycine max
  XP_003524313.1 AAP 4-like
MPENAATTNLNHLQVFGIEDDVPSHSQNNSKCYDDDGRLKRTGNVWTASSHIITAVIGSGVLSLAWAIAQLGWIAGPTVMFLFSLVTFYTSSLLADCYRAGDPNSGKRNYTYMDAVRSILGGANVTLCGIFQYLNLLGIVIGYTIAASISMMAIKRSNCFHKSGGKNPCHMSSNVYMIIFGATEIFLSQIPDFDQLWWLSTVAAIMSFTYSIIGLSLGIAKVAETGTFKGGLTGISIGPVSETQKIWRTSQALGDIAFAYSYAVVLIEIQDTIKSPPSEAKTMKKATLISIAVTTTFYMLCGCMGYAAFGDAAPGNLLTGFGFYNPYWLIDIANAAIVIHLVGAYQVFSQPIFAFVEKEVTQRWPHIEREFKIPIPGFSPYKLKVFRLVLRTVFVVLTTVISMLLPFFNDIVGVIGALGFWPLTVYFPVEMYISQKKIPKWSNRWISLKIFSVACLIVSVVAAVGSVAGVLLDLKKYKPFHSHY

SEQ ID NO: 58 - DNA, 1455 bp, Glycine max
  XP_003524313.1 AAP 4-like
atgcctgaaaacgcagccacaaccaaccttaaccaccttcaagtttttggcatcgaagatgatgtgccttcgcattcacagaataactccaaatgctatgatgatgatggccgtctcaaacgaactggaaatgtttggactgcaagctcgcacataataaccgcagtgataggatcaggggtgctgtcattagcttgggccatagctcagctaggttggattgctggtcctactgtcatgttcttattctctttggttaccttttatacttcatccttgttggctgattgttatcgtgccggtgaccccaattctggcaagagaaactacacttacatggacgcagttcgctccattcttggtggagccaatgttacgttgtgcggaatatttcagtacctgaatctattgggaattgtaataggatacacaattgccgcttctattagcatgatggcaattaaaaggtcaaactgtttccataaatctgggggcaaaaacccatgtcacatgtcaagcaacgtatacatgatcatttttggcgcaaccgaaattttcctttctcaaattcccgattttgatcaattatggtggctctcaacagttgctgcaataatgtctttcacctattccataattggtctctctcttggaattgccaaagttgcagaaacgggtaccttcaagggtggcctcactggaatcagcattggaccagtgtcagagacccaaaaaatctggaggacttcccaagctcttggtgatatagcctttgcctattcatatgctgtggttcttatagaaattcaggacacaataaaatctccaccgtctgaagcaaaaacaatgaagaaggccacattgataagtattgcagtgaccacaacattttacatgctctgtggctgcatggggtatgctgcttttggagatgctgcaccggggaatctgctaactggctttggcttctataacccatattggcttatagacattgcaaatgcagctatagttattcaccttgtgggagcataccaagtgttttcccaacccatctttgcctttgtggagaaagaggtaacacaaagatggccccacattgagagggagttcaagattccaattcctggtttctccccttacaaacttaaggtgtttagattagttttgaggacagtgtttgttgtcctaacaactgtcatatcaatgctgcttccattcttcaatgacattgttggagtgattggggcattgggattttggcccttaactgtttactttcctgtggagatgtatatttcacagaagaagatcccaaaatggagtaacagatggattagcctcaaaatatttagtgtggcctgcctcatagtatcagttgttgctgctgttggctcagtggcaggagtcttgcttgaccttaagaaatacaaaccattccactcacactattaa

SEQ ID NO: 59 - protein, 485 aa, Glycine max
  XP_006581782.1 AAP3 isoform X1
MVEKSSRTNLSHHQDFGMEPYSIDGVSSQTNSKFYDDDGHVKRTGTVWTTSSHIITAVVGSGVLSLAWAMAQMGWVAGPAVMIFFSVVTLYTTSLLADCYRCGDPVTGKRNYTFMDAVQSILGGYYDAFCGVVQYSNLYGTAVGYTIAASISMMAIKRSNCFHSSGGKSPCQVSSNPYMIGFGIIQILFSQIPDFHETWWLSIVAAIMSFVYSTIGLALGIAKVAEMGTFKGSLTGVRIGTVTEATKVWGVFQGLGDIAFAYSYSQILIEIQDTIKSPPSEAKTMKKSAKISIGVTTTFYMLCGFMGYAAFGDSAPGNLLTGFGFFNPYWLIDIANAAVIHLVGAYQVYAQPLFAFVEKWASKRWPEVETEYKIPIPGFSPYNLSPFRLVWRTVFVIITTFVAMLIPFFNDVLGLLGALGFWPLSVFLPVQMSIKQKRTPRWSGRWIGMQILSVVCFIVSVAAAVGSVASIVLDLQKYKPFHVDY

SEQ ID NO: 60 - DNA, 1461 bp, Glycine max
  XP_006581782.1 AAP3 isoform X1
atggttgaaaaatcttccagaaccaatcttagccaccatcaagactttggcatggagccttactccattgatggtgtttcttcacaaactaactccaaattctacgatgatgatggccatgttaaacgaacagggaccgtttggacaacaagctcgcacataataacagcagtggtgggttctggggtgctgtctttggcatgggccatggctcaaatgggttgggttgctgggcctgcagttatgatcttcttcagtgttgttacgttgtatacgacgtcgcttctggctgattgttatcgctgtggtgaccctgttaccgggaagagaaactatactttcatggatgcagttcaatccattctcggtgggtattatgatgcattttgtggggtagttcagtactcaaatctttacggaaccgccgtaggatacacaattgcagcttctattagcatgatggcaataaaaaggtccaactgtttccattcttcaggcggaaaaagtccatgtcaggtttcaagcaacccatacatgatcggttttggcataatccaaattttattttctcaaattccagattttcatgaaacatggtggctctccatagttgcagcaatcatgtcttttgtctattccacaattgggctcgctcttggcattgccaaagttgcagaaatgggtactttcaagggtagtctcacaggagtaaggattggaactgtgaccgaggccacaaaagtatggggggttttccaaggtcttggtgacatagccttcgcctattcatattctcaaattctcattgaaattcaggacaccataaaatctccaccatcggaagcaaagacaatgaagaagtctgctaagataagtattggagtaaccacaacattttatatgctttgtggtttcatgggctatgctgcttttggagattcagcacctgggaacctgctcacaggatttggtttttttaacccatattggctcatagatattgctaatgctgctatcgtaattcaccttgtgggagcataccaagtttatgcccagcccctctttgcctttgtcgagaaatgggcttcaaaaagatggcctgaagttgagacggaatataaaattccaattcctggtttttcaccctacaatctaagcccatttagattagtttggagaacagtgtttgttatcataaccacttttgtagcaatgttgattccattcttcaatgacgttttgggacttcttggagcactggggttttggcctttaagtgtttttctcccagtgcagatgagtatcaaacaaaagaggaccccaaggtggagtggtagatggattggtatgcaaatcttaagtgttgtttgtttcatagtatcagttgcggctgctgttggctcagttgccagtatcgtgcttgacctacagaaatacaaaccgtttcatgtagactattaa

SEQ ID NO: 61 - protein, 469 aa, Glycine max
  XP_006581783.1 AAP3 isoform X2
MEPYSIDGVSSQTNSKFYDDDGHVKRTGTVWTTSSHIITAVVGSGVLSLAWAMAQMGWVAGPAVMIFFSVVTLYTTSLLADCYRCGDPVTGKRNYTFMDAVQSILGGYYDAFCGVVQYSNLYGTAVGYTIAASISMMAIKRSNCFHSSGGKSPCQVSSNPYMIGFGIIQILFSQIPDFHETWWLSIVAAIMSFVYSTIGLALGIAKVAEMGTFKGSLTGVRIGTVTEATKVWGVFQGLGDIAFAYSYSQILIEIQDTIKSPPSEAKTMKKSAKISIGVTTTFYMLCGFMGYAAFGDSAPGNLLTGFGFFNPYWLIDIANAAIVIHLVGAYQVYAQPLFAFVEKWASKRWPEVETEYKIPIPGFSPYNLSPFRLVWRTVFVIITTFVAMLIPFFNDVLGLLGALGFWPLSVFLPVQMSIKQKRTPRWSGRWIGMQILSVVCFIVSVAAAVGSVASIVLDLQKYKPFHVDY

SEQ ID NO: 62 - DNA, 1410 bp, Glycine max
  XP_006581783.1 AAP3 isoform X2
atggagccttactccattgatggtgtttcttcacaaactaactccaaattctacgatgatgatggccatgttaaacgaacagggaccgtttggacaacaagctcgcacataataacagcagtggtgggttctggggtgctgtctttggcatgggccatggctcaaatgggttgggttgctgggcctgcagttatgatcttcttcagtgttgttacgttgtatacgacgtcgcttctggctgattgttatcgctgtggtgaccctgttaccgggaagagaaactatactttcatggatgcagttcaatccattctcggtgggtattatgatgcattttgtggggtagttcagtactcaaatctttacggaaccgccgtaggatacacaattgcagcttctattagcatgatggcaataaaaaggtccaactgtttccattcttcaggcggaaaaagtccatgtcaggtttcaagcaacccatacatgatcggttttggcataatccaaattttattttctcaaattccagattttcatgaaacatggtggctctccatagttgcagcaatcatgtcttttgtctattccacaattgggctcgctcttggcattgccaaagttgcagaaatgggtactttcaagggtagtctcacaggagtaaggattggaactgtgaccgaggccacaaaagtatggggggttttccaaggtcttggtgacatagccttcgcctattcatattctcaaattctcattgaaattcaggacaccataaaatctccaccatcggaagcaaagacaatgaagaagtctgctaagataagtattggagtaaccacaacattttatatgctttgtggtttcatgggctatgctgcttttggagattcagcacctgggaacctgctcacaggatttggtttttttaacccatattggctcatagatattgctaatgctgctatcgtaattcaccttgtgggagcataccaagtttatgcccagcccctctttgcctttgtcgagaaatgggcttcaaaaagatggcctgaagttgagacggaatataaaattccaattcctggtttttcaccctacaatctaagcccatttagattagtttggagaacagtgtttgttatcataaccacttttgtagcaatgttgattccattcttcaatgacgttttgggacttcttggagcactggggttttggcctttaagtgtttttctcccagtgcagatgagtatcaaacaaaagaggaccccaaggtggagtggtagatggattggtatgcaaatcttaagtgttgtttgtttcatagtatcagttgcggctgctgttggctcagttgccagtatcgtgcttgacctacagaaatacaaaccgtttcatgtagactattaa

SEQ ID NO: 63 - protein, 483 aa, Zea mays
  NP_001136620 uncharacterized protein LOC100216745
MVSERQQAAGKVAAFNLTEAGFGDGSDLLDDDGRERRTGTLVTASAHIITAVIGSSVLSLAWAIAQLGWVIGPVVLLAFSAITWFCSSLLADCYRAPPGPGQGKRNYTYGQAVRSYLGESKYRLCSLAQYVNLVGVTIGYTITTAISMGAIKRSNCFHSRGHGADCEASNTTNMIIFAGIQILLSQLPNFHKLWWLSIVAAVMSLAYSSIGLGLSIAKIAGGVHVKTSLTGAAVGVDVTAAEKVWKTFQSLGDIAFAYTYSNVLIEIQDTLRSSPPENVVMKKASFIGVSTTTAFYMLCGVLGYAAFGSDAPGNFLTGFGFYDPFWLIDVGNVCIAVHLVGAYQVFCQPIYQFVEAWARGRWPDCAFLHAELAVVAGSSFTASPFRLVWRTAYVVLTALVATVFPFFNDFLGLIGAVSFWPLTVYFPIQMYMAQAKTRRFSPAWTWMNVLSYACLFVSLLAAAGSVQGLVKDLKGYKPLFKVS

SEQ ID NO: 64 - DNA, 1452 bp, Zea mays
  NP_001136620 uncharacterized protein LOC100216745
atggtgtcggagaggcagcaggcggcggggaaggtggccgccttcaacctcacggaggccgggttcggcgacgggtcggacctgctggacgacgacgggcgcgagaggcgcacggggaccctggtgacggcgagcgcgcacatcatcacggcggtgatcgggtcgagcgtgctgtcgctggcgtgggcgatcgcgcagctggggtgggtgatcggccccgtggtgctgctggccttctccgccatcacctggttctgctccagcctactcgccgactgctaccgcgcgccgccgggccccggccagggcaagcggaactacacctacggacaggccgtcaggtcatacctgggggagtccaagtaccggctgtgctcgctggcgcagtacgtgaacctggtgggcgtcaccatcggctacaccatcaccacggccatcagcatgggggcgatcaagcgttccaactgcttccacagcaggggccacggcgccgactgcgaggcgtccaacaccaccaacatgatcatcttcgcgggcatccagatcctgctgtcgcagctccccaacttccacaagctctggtggctctccatcgtcgccgccgtcatgtccctcgcctactcctccatcggactcggcctctccatcgcaaagatcgcaggtggggtgcacgttaagacgtcgctgactggtgccgccgtgggggtggacgtcaccgcggccgagaaggtctggaagacgttccagtcgctgggggacatcgccttcgcctacacctactccaacgtgctgatcgagatccaggacacgctgcggtcgagcccgccggagaacgtggtgatgaagaaggcgtccttcatcggcgtgtccaccaccaccgcgttctacatgctgtgcggcgtgctgggctacgcggcgttcggcagcgacgcgccgggcaacttcctcacgggcttcggcttctacgaccccttctggctcatcgacgtcggcaacgtctgcatcgccgtgcacctggtcggcgcctaccaggtcttctgccagcccatctaccagttcgtggaggcctgggcgcggggccgctggcccgactgcgccttcctccacgccgagctcgccgtcgtcgccggctcctccttcacggccagcccgttccgcctcgtgtggcgcaccgcctacgtcgtgctcaccgcgctcgtcgccacggtcttcccattcttcaacgacttcctggggctcatcggcgccgtctccttctggccgctcaccgtctacttccccatccagatgtacatggcgcaggccaagacgcgccgcttctcgccggcgtggacgtggatgaacgtgctcagctacgcttgcctcttcgtctcgctgctcgccgccgcgggctcagtgcaggggctcgtcaaggatctcaagggatacaagccattgttcaaggtctcctaa

SEQ ID NO: 65 - protein, 489 aa, Zea mays
  PWZ15603 AAP6
MVSERQQAAGKVAAFNLTEAGFGDGSDLLDDDGRERRTGTLVTASAHIITAVIGSGVLSLAWAIAQLGWVIGPVVLLAFSAITWFCSSLLADCYRAPPGPGQGKRNYTYGQAVRSYLGESKYRLCSLAQYVNLVGVTIGYTITTAISMGAIKRSNCFHSRGHGADCEASNTTNMIIFAGIQILLSQLPNFHKLWWLSIVAAVMSLAYSSIGLGLSIAKIAGKLMHGSGVHVKTSLTGAAVGVDVTAAEKVWKTFQSLGDIAFAYTYSNVLIEIQDTLRSSPPENVVMKKASFIGVSTTTAFYMLCGVLGYAAFGSDAPGNFLTGFGFYDPFWLIDVGNVCIAVHLVGAYQVFCQPIYQFVEAWARGRWPDCAFLHAELAVVAGSSFTASPFRLVWRTAYVVLTALVATVFPFFNDFLGLIGAVSFWPLTVYFPIQMYMAQAKTRRFSPAWTWMNVLSYACLFVSLLAAAGSVQGLVKDLKGYKPLFKVS

SEQ ID NO: 66 - DNA, 1470 bp, Zea mays
  PWZ15603 AAP6
atggtgtcggagaggcagcaggcggcggggaaggtggccgccttcaacctcacggaggccgggttcggcgacgggtcggacctgctggacgacgacgggcgcgagaggcgcacggggaccctggtgacggcgagcgcgcacatcatcacggcggtgatcgggtcgggcgtgctgtcgctggcgtgggcgatcgcgcagctggggtgggtgatcggccccgtggtgctgctggccttctccgccatcacctggttctgctccagcctactcgccgactgctaccgcgcgccgccgggccccggccagggcaagcggaactacacctacggacaggccgtcaggtcatacctgggggagtccaagtaccggctgtgctcgctggcgcagtacgtgaacctggtgggcgtcaccatcggctacaccatcaccacggccatcagcatgggggcgatcaagcgttccaactgcttccacagcaggggccacggcgccgactgcgaggcgtccaacaccaccaacatgatcatcttcgcgggcatccagatcctgctgtcgcagctccccaacttccacaagctctggtggctctccatcgtcgccgccgtcatgtccctcgcctactcctccatcggactcggcctctccatcgcaaagatcgcaggcaagctcatgcatggcagtggggtgcacgttaagacgtcgctgactggtgccgccgtgggggtggacgtcaccgcggccgagaaggtctggaagacgttccagtcgctgggggacatcgccttcgcctacacctactccaacgtgctgatcgagatccaggacacgctgcggtcgagcccgccagagaacgtggtgatgaagaaggcgtccttcatcggcgtgtccaccaccaccgcgttctacatgctgtgcggcgtgctgggctacgcggcgttcggcagcgacgcgccgggcaacttcctcacgggcttcggcttctacgaccccttctggctcatcgacgtcggcaacgtctgcatcgccgtgcacctggtcggcgcctaccaggtcttctgccagcccatctaccagttcgtggaggcctgggcgcggggccgctggcccgactgcgccttcctccacgccgagctcgccgtcgtcgccggctcctccttcacggccagcccgttccgcctcgtgtggcgcaccgcctacgtcgtgctcaccgcgctcgtcgccacggtcttcccattcttcaacgacttcctggggctcatcggcgccgtctccttctggccgctcaccgtctacttccccatccagatgtacatggcgcaggccaagacgcgccgcttctcgccggcgtggacgtggatgaacgtgctcagctacgcttgcctcttcgtctcgctgctcgccgccgcgggctccgtgcaggggctcgtcaaggatctcaagggatacaagccattgttcaaggtctcctaa

SEQ ID NO: 67 - protein, 491 aa, Zea mays
  ONM51229.1 Amino acid permease 6
MVSERQQAAGKVAAFNLTEAGFGDGSDLLDDDGRERRTGTLVTASAHIITAVIGSGVLSLAWAIAQLGWVIGPVVLLAFSAITWFCSSLLADCYRAPPGPGQGKRNYTYGQAVRSYLGESKYRLCSLAQYVNLVGVTIGYTITTAISMGAIKRSNCFHSRGHGADCEASNTTNMIIFAGIQILLSQLPNFHKLWWLSIVAAVMSLAYSSIGLGLSIAKIAGKLMHGSYCGVHVKTSLTGAAVGVDVTAAEKVWKTFQSLGDIAFAYTYSNVLIEIQDTLRSSPPENVVMKKASFIGVSTTTAFYMLCGVLGYAAFGSDAPGNFLTGFGFYDPFWLIDVGNVCIAVHLVGAYQVFCQPIYQFVEAWARGRWPDCAFLHAELAVVAGSSFTASPFRLVWRTAYVVLTALVATVFPFFNDFLGLIGAVSFWPLTVYFPIQMYMAQAKTRRFSPAWTWMNVLSYACLFVSLLAAAGSVQGLVKDLKGYKPLFKVS

SEQ ID NO: 68 - DNA, 1475 bp, Zea mays
  ONM51229.1 Amino acid permease 6
atggtgtcggagaggcagcaggcggcggggaaggtggccgccttcaacctcacggaggccgggttcggcgacgggtcggacctgctggacgacgacgggcgcgagaggcgcacggggaccctggtgacggcgagcgcgcacatcatcacggcggtgatcgggtcgggcgtgctgtcgctggcgtgggcgatcgcgcagctggggtgggtgatcggccccgtggtgctgctggccttctccgccatcacctggttctgctccagctactcgccgactgctaccgcgcgccgccgggccccggccagggcaagcggaactacacctacggacaggccgtcaggtcatacctgggggagtccaagtaccggctgtgctcgctggcgcagtacgtgaacctggtgggcgtcaccatcggctacaccatcaccacggccatcagcatgggggcgatcaagcgttccaactgcttccacagcaggggccacggcgccgactgcgaggcgtccaacaccaccaacatgatcatcttcgcgggcatccagatcctgctgtcgcagctccccaacttccacaagctctggtggctctccatcgtcgccgccgtcatgtccctcgcctactcctccatcggactcggcctctccatcgcaaagatcgcaggcaagctcatgcatggcagctactgtggggtgcacgttaagacgtcgctgactggtgccgccgtgggggtggacgtcaccgcggccgagaaggtctggaagacgttccagtcgctgggggacatcgccttcgcctacacctactccaacgtgctgatcgagatccaggacacgctgcggtcgagcccgccggagaacgtggtgatgaagaaggcgtccttcatcggcgtgtccaccaccaccgcgttctacatgctgtgcggcgtgctgggctacgcggcgttcggcagcgacgcgccgggcaacttcctcacgggcttcggcttctacgaccccttctggctcatcgacgtcggcaacgtctgcatcgccgtgcacctggtcggcgcctaccaggtcttctgccagcccatctaccagttcgtggaggcctgggcgcggggccgctggcccgactgcgccttcctccacgccgagctcgccgtcgtcgccggctcctccttcacggccagcccgttccgcctcgtgtggcgcaccgcctacgtcgtgctcaccgcgctcgtcgccacggtcttcccattcttcaacgacttcctggggctcatcggcgccgtctccttctggccgctcaccgtctacttccccatccagatgtacatggcgcaggccaagacgcgccgcttctcgccggcgtggacgtggatgaacgtgctcagctacgcttgcctcttcgtctcgctgctcgccgccgcgggctcagtgcaggggctcgtcaaggatctcaagggatacaagccattgttcaaggtctcctaa

SEQ ID NO: 69 - protein, 486 aa, Zea mays
  NP_001349744.1 uncharacterized protein LOC100501686
MTQQDVEMAARHGTGADGAGFYPQPRNGAGGETLDDDGKKKRTGTVWTASAHIITAVIGSGVLSLAWSTAQLGWVVGPLTLMIFALITYYTSSLLADCYRSGDQLTGKRNYTYMDAVAAYLGRWQVLSCGVFQYVNLVGTAVGYTITASISAAAVHKANCFHNKGHAADCSTYDTMYMVVFGIVQIFFSQLPNFSDLSWLSIVAAIMSFSYSSIAVGLSLARTISGRSGTTTLTGTEIGVDVDSAQKVWLALQALGNIAFAYSYSMILIEIQDTVKSPPAENKTMKKATLMGVTTTTAFYMLAGCLGYSAFGNAAPGNILTGFGFYEPYWLIDFANVCIVVHLVGAYQVFSQPIFAALETAAAKRWPNARFVTREHPLVAGRFHVNLLRLTWRTAFVVVSTVLAIVLPFFNDILGFLGAIGFWPLTVYYPVEMYIRQRRIQKYTSRWVALQLLSFLCFLVSLASAVASIEGVTESLKHYVPFKTKS

SEQ ID NO: 70 - DNA, 1461 bp, Zea mays
  NP_001349744.1 uncharacterized protein LOC100501686
atgacgcagcaggacgtggagatggcggcgcgccacgggaccggcgccgacggagcgggattctaccctcagccgcggaacggcgccggcggcgagacgctcgacgacgacggcaagaagaagcgcacgggaacggtatggacggcaagcgcgcacatcatcacagccgtcatcggctccggcgtgctctccctcgcctggtcgactgcacagctgggctgggtcgtggggccgctcaccctgatgatctttgccttgatcacgtactacacctctagccttcttgctgactgctaccgcagcggcgatcagctcaccggcaagaggaactacacctacatggacgctgttgccgcgtacctgggtcgatggcaagtcctgtcctgtggtgttttccagtatgttaacttggttggaactgccgttgggtatacaattacagcgtccatcagtgcagcggccgtgcacaaggcaaactgcttccacaacaagggccacgcggccgactgcagcacctacgacaccatgtacatggtcgtatttgggatcgttcagatcttcttctctcagctccctaacttcagcgacctttcgtggctgtccatcgtcgccgccatcatgtcgttctcttactccagcatcgccgtcggcctctcgttggcgcggaccatttcaggccgtagtggtacgaccactctgaccggcactgagatcggagtcgacgttgattcagcccagaaggtctggctcgcgcttcaagctcttggcaacatcgcgttcgcttactcctactccatgattctcatcgaaatccaagacacggtgaagtctcctccagccgagaacaagacgatgaagaaggcgacgctgatgggcgtgacgaccaccacggcgttctacatgcttgctggctgcctcgggtactcggcattcgggaacgcggcgccagggaacatcctgaccgggttcggcttctacgagccctactggctgatcgacttcgccaacgtctgcatcgtggtgcacctggtgggcgcgtaccaggtcttctcccagcccatcttcgcggccttggagacggcggccgccaagcgctggccgaacgccaggttcgtcacgcgcgagcaccccctcgtggccggcaggttccacgtcaacctgctcaggctgacgtggaggacggcgttcgtggtggtgagcacggtgctcgccatcgtgttgcccttcttcaacgatatcctgggcttcctcggcgccatcggcttctggccgctcaccgtgtactacccagtggagatgtacatccggcagcggcgtatacagaagtacaccagcaggtgggtggcgctgcagctgctcagcttcctgtgcttcctggtctcgctcgcctcggcggtcgcgtccatcgagggagtcaccgagtcgctcaaacactacgttccctttaagaccaagtcgtga

SEQ ID NO: 71 - protein, 477 aa, Zea mays
  PWZ08709 AAP3
MEVSSVEFGHAAAASKCFDDDGRLKRTGTMWTASAHIITAVIGSGVLSLAWAIAQLGWVAGPTVMLLFSFVTYYTSALLADCYRSGDACTGKRNYTYMDAVNANLSGVKVWFCGFLQYANIVGVAIGYTIAASISMLAIQRANCFHVEGHGDPCNISSTPYMIIFGVVQIFFSQIPDFDQISWLSILAAVMSFTYSTIGLGLGIAQVVSNKGVQGSLTGISVGAVTPVDKMWRSLQAFGDIAFAYSYSLILIEIQDTIRAPPPSESKVMRRATVVSVAVTTFFYMLCGCMGYAAFGDNAPGNLLTGFGFYEPFWLLDVANAAIAVHLVGAYQVYCQPLFAFVEKWARQRWPKSRYITGEVDVPLPLGTAAGRCYKLSLFRLTWRTAFVVATTVVSMLLPFFNDVVGLLGALGFWPLTVYFPVEMYIVQKKVPRWSTRWVCLQLLSVACLVITVASAAGSVAGIVSDLKVYKPFVTTS

SEQ ID NO: 72 - DNA, 1434 bp, Zea mays
  PWZ08709.1 AAP3
atggaggtgagctccgtggagttcggtcacgcggcggccgcctcaaagtgctttgacgacgacggtcgcctcaagcgcacagggacgatgtggacggcgagcgcgcacattatcacggccgtgatagggtccggggtgctgtcgctcgcgtgggccatcgcgcagctcggctgggtggcaggccccaccgtcatgctgctcttctccttcgtcacctactacacgtcggccctactcgccgactgctaccgctccggcgacgcctgcaccggcaagcgcaactacacgtacatggacgcggttaacgccaatctcagtggcgtcaaggtttggttctgcgggttcctgcagtacgccaacatcgtcggagtcgccataggctacaccattgccgcctctattagcatgctggcgatccagagggcgaactgcttccacgtggaggggcacggggacccctgcaacatctccagcacgccctacatgatcatcttcggcgtcgtgcagattttcttctcgcagatcccggacttcgaccagatatcgtggctctccatcctcgccgccgtcatgtccttcacctactccaccatcggcctgggcctgggcatcgcgcaggtggtgtccaacaagggcgtgcagggcagcctgacggggatcagcgtcggcgcggtcaccccggtcgacaagatgtggcgcagcctgcaggcgttcggcgacatcgccttcgcctactcctactccctcatcctcatcgagatccaggacaccatccgcgcgccgccgccgtccgagtccaaggtcatgcggcgcgccaccgtcgtcagcgtggccgtcaccacgttcttctacatgctgtgcgggtgcatggggtacgccgcgttcggggacaacgcccccgggaacctcctcacgggcttcggcttctacgagcccttctggctcctcgacgtcgccaacgccgccatcgccgtgcacctcgtcggcgcctaccaggtctactgccagccactgttcgccttcgtcgagaagtgggcgcgccagaggtggcccaagtcccgctacatcacgggcgaggtcgacgtcccgctcccgctcgggaccgccgccggccggtgctacaagctcagcctgttccggctgacgtggcggacggcgttcgtggtggccacgacggtggtgtccatgctgctgcccttcttcaacgacgtggtcgggctcctgggcgcgctggggttctggccgctcaccgtctacttccccgtggagatgtacatcgtgcagaagaaggtgcccaggtggagcacgcggtgggtgtgcctgcagctgctcagcgtcgcctgcctcgtcatcaccgtcgcctccgccgcaggctccgttgccgggatcgtctctgacctcaaagtgtacaaaccgttcgtcaccacctcctga

SEQ ID NO: 73 - protein, 478 aa, Zea mays
  NP_001149036 amino acid carrier
MEVSSVEFGHHAAAASKCFDDDGRLKRTGTMWTASAHIITAVIGSGVLSLAWAIAQLGWVAGPTVMLLFSFVTYYTSALLADCYRSGDACTGKRNYTYMDAVNANLSGVKVWFCGFLQYANIVGVAIGYTIAASISMLAIQRANCFHVEGHGDPCNISSTPYMIIFGVVQIFFSQIPDFDQISWLSILAAVMSFTYSTIGLGLGIAQVVSNKGVQGSLTGISVGLVTPVDKMWRSLQAFGDIAFAYSYSLILIEIQDTIRAPPPSESKVMRRATVVSVAVTTFFYMLCGCMGYAAFGDNAPGNLLTGFGFYEPFWLLDVANAAIAVHLVGAYQVYCQPLFAFVEKWARQRWPKSRYITGEVDVPLPLGTAGGRCYKLSLFRLTWRTAFVVATTVVSMLLPFFNDVVGLLGALGFWPLTVYFPVEMYIVQKKVPRWSTRWVCLQLLSVACLVITVASAAGSVAGIVSDLKVYKPFVTTS

SEQ ID NO: 74 - DNA, 1436 bp, Zea mays
  NP_001149036 amino acid carrier
atggaggtgagctccgtggagttcggtcatcacgcggcggccgcctcaaagtgctttgacgacgacggtcgcctcaagcgcacagggacgatgtggacggcgagcgcgcacattatcacggccgtgatagggtccggggtgctgtcgctcgcgtgggccatcgcgcagctcggctgggtggcaggccccaccgtcatgctgctcttctccttcgtcacctactacacatcggccctactcgccgactgctaccgctccggcgacgcctgcaccggcaagcgcaactacacgtacatggacgcggttaacgccaatctcagtggcgtcaaggtctggttctgcgggttcctgcagtacgccaacatcgtcggagtcgccataggctacaccattgccgcctctattagcatgctggcgatccagagggcgaactgcttccacgtggaggggcacggggacccctgcaacatctcagcacgccctacatgatcatcttcggcgtcgtgcagattttcttctcgcagatcccggacttcgaccagatatcgtggctctccatcctcgccgccgtcatgtcgttcacctactccaccatcggcctgggcctgggcatcgcgcaggtggtgtccaacaagggcgtgcagggcagcctgacggggatcagcgtcggcttggtcaccccggtcgacaagatgtggcgcagcctgcaggcgttcggcgacatcgccttcgcctactcctactcgctcatcctcatcgagatccaggacaccatccgcgcgccgccgccgtccgagtccaaggtcatgcggcgcgccaccgtcgtcagcgtggccgtcaccacgttcttctacatgctgtgcgggtgcatggggtacgccgcgttcggggacaacgcccccgggaacctcctcacgggcttcggcttctacgagcccttctggctcctcgacgtcgccaacgccgccatcgccgtgcacctcgtcggcgcctaccaggtctactgccagcccctgttcgccttcgtcgagaagtgggcgcgccagaggtggcccaagtcccgctacatcacgggcgaggtcgacgtcccgctcccgctcgggaccgccggcggccggtgctacaagctcagcctgttccggctgacgtggcggacggcgttcgtggtggccacgacggtggtgtccatgctgctgcccttcttcaacgacgtggtcgggctcctgggcgcgctggggttctggccgctcaccgtctacttccccgtggagatgtacatcgtgcagaagaaggtgcccaggtggagcacgcggtgggtgtgcctgcagctgctcagcgtcgcctgcctcgtcatcaccgtcgcctccgccgcaggctccgttgccgggatcgtctctgacctcaaagtgtacaaaccgttcgtcaccacctcctga

SEQ ID NO: 75 - protein, 478 aa, Zea mays
  ACG33909.1 amino acid carrier
  UNSURE 284: X is an unknown amino acid
MEVSSVEFGHHAAAASKCFDDDGRLKRTGTMWTASAHIITAVIGSGVLSLAWAIAQLGWVAGPTVMLLFSFVTYYTSALLADCYRSGDACTGKRNYTYMDAVNANLSGVKVWFCGFLQYANIVGVAIGYTIAASISMLAIQRANCFHVEGHGDPCNISSTPYMIIFGVVQIFFSQIPDFDQISWLSILAAVMSFTYSTIGLGLGIAQVVSNKGVQGSLTGISVGAVTPVDKMWRSLQAFGDIAFAYSYSLILIEIQDTIRAPPPSESKVMRRATVVSVAVTTFXYMLCGCMGYAAFGDNAPGNLLTGFGFYEPFWLLDVANAAIAVHLVGAYQVYCQPLFAFVEKWARQRWPKSRYITGEVDVPLPLGTAGGRCYKLSLFRLTWRTAFVVATTVVSMLLPFFNDVVGLLGALGFWPLTVYFPVEMYIVQKKVPRWSTRWVCLQLLSVACLVITVASAAGSVAGIVSDLKVYKPFVTTS

SEQ ID NO: 76 - DNA, 1437 bp, Zea mays
  ACG33909.1 amino acid carrier
atggaggtgagctccgtggagttcggtcatcacgcggcggccgcctcaaagtgctttgacgacgacggtcgcctcaagcgcacagggacgatgtggacggcgagcgcgcacattatcacggccgtgatagggtccggggtgctgtcgctcgcgtgggccatcgcgcagctcggctgggtggcaggccccaccgtcatgctgctcttctccttcgtcacctactacacatcggccctactcgccgactgctaccgctccggcgacgcctgcaccggcaagcgcaactacacgtacatggacgcggttaacgccaatctcagtggcgtcaaggtctggttctgcggcttcctgcagtacgccaacatcgtcggagtcgccataggctacaccattgccgcctctattagcatgctggcgatccagagggcgaactgcttccacgtggaggggcacggggacccctgcaacatctccagcacgccctacatgatcatcttcggcgtcgtgcagattttcttctcgcagatcccggacttcgaccagatatcgtggctctccatcctcgccgccgtcatgtccttcacctactccaccatcggcctgggcctgggcatcgcgcaggtggtgtccaacaagggcgtgcagggcagcctgacggggatcagcgtcggcgcggtcaccccggtcgacaagatgtggcgcagcctgcaggcgttcggcgacatcgccttcgcctactcctactccctcatcctcatcgagatccaggacaccatccgcgcgccgccgccgtccgagtccaaggtcatgcggcgcgccaccgtcgtcagcgtggccgtcaccacgttcttmtacatgctgtgcgggtgcatggggtacgccgcgttcggggacaacgcccccgggaacctcctcacgggcttcggcttctacgagcccttctggctcctcgacgtcgccaacgccgccatcgccgtgcacctcgtcggcgcctaccaggtctactgccagcccctgttcgccttcgtcgagaagtgggcgcgccagaggtggcccaagtcccgctacatcacgggcgaggtcgacgtcccgctcccgctcgggaccgccggcggccggtgctacaagctcagcctgttccggctgacgtggcggacggcgttcgtggtggccacgacggtggtgtccatgctgctgcccttcttcaacgacgtggtcgggctcctgggcgcgctggggttctggccgctcaccgtctacttccccgtggagatgtacatcgtgcagaagaaggtgcccaggtggagcacgcggtgggtgtgcctgcagctgctcagcgtcgcctgcctcgtcatcaccgtcgcctccgccgcaggctccgttgccgggatcgtctctgacctcaaagtgtacaaaccgttcgtcaccacctcctga

SEQ ID NO: 77 - protein, 494 aa, Zea mays
  NP_001142349 AAP2
MAENNVVATYYYPTAAPAAMEVCGAELGQGKPDKCFDDDGRPKRNGTMWTASAHIITAVIGSGVLSLGWAIAQLGWVAGPVVMLLFSLVTYYTSSLLADCYRSGDPSTGKRNYTYMDAVNANLSGIKVQICGFLQYANIVGVAIGYTIAASISMLAIRRANCFHQKGHGNPCKISSTPYMIIFGVAEIFFSQIPDFDQISWLSILAAVMSFTYSSIGLGLGVVQVIANRGVQGSLTGITIGVVTPMDKVWRSLQAFGDVAFAYSYSLILIEIQDTIRAPPPSESTVMKRATVVSVAVTTLFYMLCGCMGYAAFGDGAPGNLLTGFGFYEPFWLLDVANAAIVVHLVGAYQVYCQPLFAFVEKWAAQRWPDSAYITGEVEVPLPLPASRRRCCKVNLFRATWRTAFVVATTVVSMLLPFFNDVVGFLGALGFWPLTVYFPVEMYVVQKKVPRWSSRWVCLQMLSLGCLVISIAAAAGSIAGIASDLKVYRPFKSY

SEQ ID NO: 78 - DNA, 1485 bp, Zea mays
  NP_001142349 AAP2
atggcggagaacaacgtcgtggccacgtactactacccgacggcagcgccggcggccatggaggtctgcggcgcggagctcggccagggcaagcccgacaagtgcttcgacgacgatggccgccccaagcgcaatgggacgatgtggacggcgagcgcgcacatcatcacggcggtgatcggctccggggtgctctcgctggggtgggccatcgcgcagctcggctgggtggccggacccgtcgtcatgctgctcttctcgctcgtcacctactacacctcgtcgctgctcgcagactgctaccgctccggcgaccccagcaccggcaagcggaactacacctacatggacgccgtcaacgcgaacctcagtggcatcaaggtccagatctgcgggttcctgcagtacgccaacatcgtgggcgtggccatcggctacaccatcgctgcctccattagcatgctcgcgatcaggagggccaactgcttccaccagaagggacacggcaacccctgcaagatctccagcacgccctacatgatcatcttcggcgtggcggagatcttcttctcgcagatcccggacttcgaccagatctcctggctctccatcctcgccgccgtcatgtccttcacctactcctccattgggctcggcctgggcgtcgtccaagtcatcgcgaacagaggcgtgcagggcagcctgaccggcatcaccatcggcgtggtgaccccgatggacaaggtgtggcgcagcctccaggcgttcggcgacgtcgccttcgcctactcctactccctcatcctgatcgagatccaggacaccatccgggcgccgccgccgtcggagtcgacggtgatgaagcgcgccacggtggtgagcgtggcggtcaccacgctcttctacatgctgtgcggctgcatggggtacgcggcgttcggcgacggcgcgcccgggaacctcctcacgggcttcggcttctacgagcccttctggctcctggacgtggccaacgccgccatcgtggtccacctggtcggcgcctaccaggtctactgccagccgctgttcgccttcgtggagaagtgggccgcgcagcggtggccggactcggcgtacatcaccggggaggtcgaggtcccgctcccgctcccggcgagccggcggcggtgctgcaaggtgaacctgttccgggcgacgtggcggacggcgttcgtcgtggccacgacggtcgtgtccatgctgctgcccttcttcaacgacgtggtgggcttcctgggcgcgctcggcttctggccgctcaccgtctacttccccgtcgagatgtacgtggtgcagaagaaggtgccgcggtggagctcccggtgggtgtgcctgcagatgctcagcctcggctgcctcgtcatctccatcgccgccgcagccgggtccatcgccggcatcgcgtccgacctcaaagtctaccgcccgttcaagtcctactga

SEQ ID NO: 79 - protein, 522 aa, Zea mays
  PWZ07549 AAP1
MTQQDVEMAARHGTGADGAGFYPQPRNGAGGETLDDDGKKKRTGVIATIGGVPSTGANVPPNVGVLDEPGTDAMPLMRPRTVWTASAHIITAVIGSGVLSLAWSTAQLGWVVGPLTLMIFALITYYTSSLLADCYRSGDQLTGKRNYTYMDAVAAYLGRWQVLSCGVFQYVNLVGTAVGYTITASISAAAVHKANCFHNKGHAADCSTYDTMYMVVFGIVQIFFSQLPNFSDLSWLSIVAAIMSFSYSSIAVGLSLARTISGRSGTTTLTGTEIGVDVDSAQKVWLALQALGNIAFAYSYSMILIEIQDTVKSPPAENKTMKKATLMGVTTTTAFYMLAGCLGYSAFGNAAPGNILTGFGFYEPYWLIDFANVCIVVHLVGAYQVFSQPIFAALETAAAKRWPNARFVTREHPLVAGRFHVNLLRLTWRTAFVVVSTVLAIVLPFFNDILGFLGAIGFWPLTVYYPVEMYIRQRRIQKYTSRWVALQLLSFLCFLVSLASAVASIEGVTESLKHYVPFKTKS

SEQ ID NO: 80 - DNA, 1569 bp, Zea mays
  PWZ07549 AAP1
atgacgcagcaggacgtggagatggcggcgcgccacgggaccggcgccgacggagcgggattctaccctcagccgcggaacggcgccggcggcgagacgctcgacgacgacggcaagaagaagcgcacgggtgtaatagccactattggaggtgtaccaagcactggtgcaaatgttccgcctaatgttggtgtccttgatgagcctggcactgatgctatgccactcatgcgccctagaacggtatggacggcaagcgcgcacatcatcacagccgtcatcggctccggcgtgctctccctcgcctggtcgactgcacagctgggctgggtcgtggggccgctcaccctgatgatctttgccttgatcacgtactacacctctagccttcttgctgactgctaccgcagcggcgatcagctcaccggcaagaggaactacacctacatggacgctgttgccgcgtacctgggtcgatggcaagtcctgtcctgtggtgttttccagtatgttaacttggttggaactgccgttgggtatacaattacagcgtccatcagtgcagcggccgtgcacaaggcaaactgcttccacaacaagggccacgcggccgactgcagcacctacgacaccatgtacatggtcgtatttgggatcgttcagatcttcttctctcagctccctaacttcagcgacctttcgtggctgtccatcgtcgccgccatcatgtcgttctcttactccagcatcgccgtcggcctctcgttggcgcggaccatttcaggccgtagtggtacgaccactctgaccggcactgagatcggagtcgacgttgattcagcccagaaggtctggctcgcgcttcaagctcttggcaacatcgcgttcgcttactcctactccatgattctcatcgaaatccaagacacggtgaagtctcctccagccgagaacaagacgatgaagaaggcgacgctgatgggcgtgacgaccaccacggcgttctacatgcttgctggctgcctcgggtactcggcattcgggaacgcggcgccagggaacatcctgaccgggttcggcttctacgagccctactggctgatcgacttcgccaacgtctgcatcgtggtgcacctggtgggcgcgtaccaggtcttctcccagcccatcttcgcggccttggagacggcggccgccaagcgctggccgaacgccaggttcgtcacgcgcgagcaccccctcgtggccggcaggttccacgtcaacctgctcaggctgacgtggaggacggcgttcgtggtggtgagcacggtgctcgccatcgtgttgcccttcttcaacgatatcctgggcttcctcggcgccatcggcttctggccgctcaccgtgtactacccagtggagatgtacatccggcagcggcgtatacagaagtacaccagcaggtgggtggcgctgcagctgctcagcttcctgtgcttcctggtctcgctcgcctcggcggtcgcgtccatcgagggagtcaccgagtcgctcaaacactacgttccctttaagaccaagtcgtga

SEQ ID NO: 81 - protein, 478 aa, Brassica napus
  AKE34780 AAP8
MKSLDTLHNPSAVESGNAAVKNVDDDGREKRTGTFLTASAHIITAVIGSGVLSLAWALAQLGWVAGTMILVIFAIITYYTSTLLADCYRAPDPITGTRNYTYMGVVRAYLGGKKVQLCGLAQYGNLVGVSIGYTITASISLVAIGKANCFHGKGHGAKCTASNYPYMVAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNIAPGDFLTDFGFYEPFWLVIFANVCIAVHLVGAYQVYVQPFFQFVESKCNKKWPESNFINKEYSLKIPLLGKFRVNHFRLVWRTNYVILTTFIAMIFPFFNSILGLLGALAFWPLTVYFPVAMHIAQTKVKKYSGRWLALNLLVLVCLIVSALAAVGSIVGLINNVKKYKPFESID

SEQ ID NO: 82 - DNA, 2952 bp, Brassica napus
  AKE34780 AAP8
atgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacgtctactttgctcgccgattgctacagagcgccggaccccatcaccggaacacgcaactacacgtacatgggcgtcgttcgagcttaccttggtatgcaatttattaccattatcctttaaatcatttttatcagcattattcaagattttttcagttttatatagaattatgtttctgatcaacaatttagtctactgatattaataatttttggtattatatggtaaaaatattcttgtgaagatacatttttgatcttttcctatttttttcataagatggtcccaggaacaaattaagatcaaagtaatgttttcttgcaagatcaaagtaatcaaccatttttagtgtatcctatcttttgaggaacattattttgtggttctaaatttttttattttgaaaattctgcatgctcttcttgggaagatatatgagttaattatcaaaatctacaaaaagataaaataattatgaaatttatcttctttccaaaattacttaatgaattgaattgactagtgtaggattatctcattaaagaatgctatcattaaatttgattgttggcctcccaaaaaaaaattgaattcaaaatgagagattgatccaaacttatccacaaaaacaaaagattatccgactttttaacatcaaaggattaatgacaataaacaatttcgatgcttaagtcctgcttcgtgtaatcgctgctgttgattgacaaaaacaaagactcctatgtaatttagaagagtaactaagttttagaatactccataacataaaacgacaggtggtaaaaaggtgcagctatgtggactagcacagtacggaaacctcgttggggtctctattggttacaccatcactgcctccataagcttagtgtaagtcgttacacaactgataacttatttcggttacaatttttgtgatggccactaaaacatgtgtgttgtgttgctttggctagagcgattgggagagcaaattgttttcatgacaagggacatggtgcgaaatgtaccgcatcgaattatccatacatggtggcatttggcggcctccagattcttctaagtcagattcctaattttcacaagctatctttcctctcaatcattgccgcggttatgtccttctcttatgcatctattggtatcggtctggccatcgccaaagtggcaagtacggtttcatttcctttattcaacgtttaaatgtttacatgaatcttcaatgttcgatcgagctaacggtggaagtggtataattatgtggaaggtgggaaggttggtaagacaacactgacaggtacggtgataggagtggacgtatctgcgtctgataaagtgtggaaagcgtttcaagcggttggggatattgcgttttcgtacgcttacaccactattctcattgagatacaggtatgtactactttcttctatcgttctttgaatatttgcatttacatagtagtcatatatatgatgtattagttagtgtatggatctattaatgttttttttttgctcttagaagtaatctctaactaccgattatggatatattagttagagaatatggatctactaccacttctattctctctttcaccaaaaagggataaagaagaaggtggcatttaccttgaagataagatgttactatcaactagagtattagcctagtaggcatgcatctacaaaaaggcttgatggattttttaattatatatgtgaagctctaaagatactgaagctcaaatatgtttttattttttttctttgttgcaggacacattgagatcaagcccaccagagaacaaagtgatgaagaaagcaagtcttattggagtctcaaccacaactgttttctacctcttatgtggttgcattgggtatgctgcattcggaaacttatcccctggtgacttccttaccgactttgggttttacgaacctttctggctcgtcattttcgccaatgtttgcattgctgtccatttagtaggtgcctatcaggtaattgtagcattaacaattatatctcttaaagtaaacctatcattgacaaacaaacatgagaacgtagcacacatttaaaaagcaaaaacagctaattatcacaacacacttgtaatcttcttaaatattcttgtgttatcctctgttttagaaatttagattaatagtcgaaattagtagaaatagagttagtttggtttaaaatataatcattttaatgatatcttcttaatcctagtttaatatcatttgcaggtatatgttcagccttttttccagtttgttgagagcaaatgtaacaaaaagtggcctgaaagcaatttcatcaacaaagaatactcgttgaagataccattgctcggaaaatttcgtgtcaacttcttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgctggtttgcttgattgtctccgccctagctgctgtgggatccattgttggcctaatcaataatgtcaagaaatacaagcctttcgagagtatagactaa

SEQ ID NO: 83 - protein, 507 aa, Brassica napus
  BnaA01g21750D
MKGFNTEQDHPAAESGNVYDVSDPTKNVDDDGREKRTGTWLTASAHIITAVIGSGVLSLAWAIAQLGWIAGTLILVIFSFITYFTSTMLADCYRAPDPVTGKRNYTYMDVVRSYLGGRKVQLCGVAQYGNLIGITVGYTITASISLVAVGKANCFHKKGHEADCTISNYPYMAVFGIIQIILSQIPNFHKLSFLSLMAAVMSFTYATIGIGLAIATVAGGKVGKTNMTGTVVGVDVTAAQKIWRSFQAVGDIAFAYAYATVLIEIQASFHIKYLWNLVSFEYEPLDRIVDTLKSSPAENKSMKRASLVGVSTTTFFYILCGCLGYAAFGNKAPGDFLTDFGFYEPFWLIDFANACIAFHLIGAYQVFAQPIFQFVEKRCNRNWPDNKFITSEYSVNVPFLGKFNISLFRLVWRTAYVVITTVVAMIFPFFNAILGLIGAASFWPLTVYFPVEMHIAQTKVKKYSPRWIGLKMLCWVCLIVSLLAAAGSIAGLISSVKTYKPFRTIHE

SEQ ID NO: 84 - DNA, 3995 bp, Brassica napus
  BnaA01g21750D
ctacaattagacttcttcgtttactctctctctctctctctctgacttacacatttacttatcttcttctctctgtcaacctcctttgctctcttctctcactatgaagagtttcaacaccgatcaacacggtcactccgcggcggaatccggcgacgtctacgccatgtccgatccaacaaagaacgttgatgacgatggccgagagaagaggacggggacgtggcttacggcgagtgcgcatattatcacggcggtaataggctcaggagtgttgtcgttggcatgggccatagctcagcttggttggatagcagggactttgattctgatcattttctcgtttatcacgtacttcacttctacgatgctcgctgattgctaccgtgcgccggatcccctcaccggaaaacgaaactacacttacatggacgttgttcgatcttacctcggtatggttttttctttgtttttttttttttaactttttactgtaaagaagattgaaaaatagacactttttagaccaacccttttttgaaaagggttctataccaaaaattcaattttgttaagtaatttttaaggcatggttggttccttttccaaaaaaaaagagagaggataacataaggttctctttcattaaaataatatactgattttgtctggtatttagttaggctgatccttgtctggaaaactattgtggtcagaataggaaaataattattagtattaaaaatttatactaatattaattaaaaatgacaaaatatatattagtaataaaaacatataatataatttataagcgacagtaacttttaggtcaatttgaaataaaactggttatgtaatgagtttatatagaacaatgatggtggtttataaaatagttgatgttacgaactataagatcaatcataagaaatcatcattgatatcttttgaaacaatcgaaaagcttatgcatccgatgagttgtggttaggaatgtagataaagtaatggatttatagattttctaacatttcctgccaatttggttttacagaagaaaaaaaatctatcgggcatataaattactgttgcgtataaattgattttttttgtagacgcttataaattgatatatatccttttaaatatttaaatttaactgaatataaaaacagtggtaaccgttcttttaattttctagttagaaaaaatgttgttgaaataattaaaggcttggttattatttatacatggattatattcgtaagcaatttaaagtttgttgtctctagtttaataaacgatgattttttacgaattctttttattaagtaaaaacactgagttattgacaaaaaaaaagagtaaaacaactgagttcaatgtaaagtgtggtaaccgtccttttaattttctaatgataaactatggttgttgaaaaattaattagacttcgctattattttcataaggattacatacttcaaataatttagagtatgttgtctagttcaataaacaatgaggttttacttcaaatcattttaagtaaaacattgaactgaacgcaaaagtgtggtaaccgtcctttaatttccaactatacaacggttgttgaaataattaattaggctgaaatttttcttaaataactcacggttttgtactgtttataagatccaaaactgtgcaacccgaggaaaacagaaattaaataatagtttgacatatgaggcaaagggttttggtagtattagcattattgttggtatttgatgttgatggagtattatatatactagtatagttccgtggagtgttttatgtttgatgtatgaaacagaagattaattaatagcttaaaaacaaatgtaataaataatcatagactgaaataatacattggaattattgaaaattgacaggtggtaggaaagtgcagctttgtggagtggcacagtatgggaatctgataggaatcactgttgggtacaccatcactgcttctattagtttggtgtaagtcaactatgtgtgaatatgtgattttactttatgaccgttaccaaatgtacagtttagttatctaccatattgatgtgatcagtatggtttcttgcgcagagcgatcgggaaagcgaactgttaccacaataaagggcaccatgcagattgtacaatatcgaactatccatatatggcggccttcgggatcattcagatccttcttagccagatccccaactttcacaagctctcttttctctcccttatggctgcggttatgtctttcgcttacgcaagtattgggattggccttgccatcgcgacggtcgcaggtacatatacaaatctttgaagatttaattagttgcaaaacaacaactactaacaattattggtcatcaagttgtagttttcaaagtacctatatcaataattgtaacaagatagataatacaataaagtaacggttgatatgttacgatatgaaaaggtgggaaagtgggtaagacgaatatgacgggaacggtggtaggagttgatgtaactgcggctcagaagatatggagatcgtttcaagcggttggagacatagcgtttgcatatgcttacgccacggttctcattgagattcaggcaagttaaccatcaatcaattttactattgagtcttgtttgctttgaatatgaacctcttaatagaattgtggtttgaaattattgacaatgacaatcaattctctatggaccacttttaatacaaagggtaaaaagaagagaatctgtcttttagcttaaaggtataacatgtgcttattagtgacaagatgtcacattcaaagacagcaaacaatgatatcaatggactttagcttaatgagttgacaatatagttaaaattttgttgtctcttaatgatattagcgttcacctttctagtgtacatgcatttagttcaatagagtgtatatgtcgactagaaagtgacggcttaataagatttaagttaaacacatgagacaaaactggatttgcacacactaaccggtgaaaatttgatactttttgcaggacacattgagatctagccccgcagagaacaaagctatgaaaagagcaagttttgtgggagtatcaaccaccactttcttctacatcttatgtggttgtcttggatatgctgcatttggaaacaaagcccctggtgatttcctcacagatttcggattctacgagccattttggctcattgactttgcaaacgcttgcattgctttccatctcattggtgcctatcaggtaaaaccaaatccaaaaggagaaaaagattgttttctttttgcactttctcgcagtttagctaatgaaaaagaaacatacttcttgcaggtgttcgcgcagcccatattccagtttgttgagaagaaatgcaatagaaactggcctgacaacaagttcatcacatctgaatattcagtaaacataccattccttggaaaattcaacatcaacctcttcagactagtgtggaggacagcttatgtggttataacaactttagtagctatgatattccctttcttcaacgccatcttgggtcttatcggagcagcttccttctggcctttaactgtttatttccccgtggagatgcacatagcacaaactaaggttaagaaatactctcctagatggattgggctgaaaatgttgtgctgggtttgcttgatcgtctccctgttagctgctgctggatccatcgctggactgataagtagtgtgaagacatacaagcccttccggactatccatgagtgagtttgagatccatgcatgagagtgaaaaaatgtaacagtttggtattttctttgtaactgtttggtgtctaaaatccaaatgaaaatattgtattgcttaaaaagctacatgaatgtctatgtatcatccacaactttcagtgtaatacatatgacccaataatagcttt

SEQ ID NO: 85 - protein, 466 aa, Brassica napus
  BnaA06g38000D
MKSFDAVHNPSAVESADANVDDDGREKRTGTLMTASAHIITAVIGSGVLSLAWAIAQLGWVAGTLILVTFAVVNYYTSTMLADCYRSDAGARNYTYMDVVRSYLGGRKVQLCGLAQYGCLVGVTIGYTITASISLVAIWKATCFHKKGHGAKCSIPNYPFMVAFGVVEIFLSQLPNFHKLSFLSIIAAIMSFSYASIGIGLAISVVASGKVGKTSVTGTVVGVDVTASDKIWKAFQATGDIAFSYSFSTILDTLRSNPPENKVMKKATLAGVSTTTVFYILCGCMGYAAFGNRAPGDFLTDFGFYEPYWLINFANACIVLHLIAAYQVFAQPIFQLVENKCNKAWPENNFIHKEHSINILFLGKWRINFFRLVWRTAYVILTTFVAVIFPFFNSILGLIGATAFWPLTVYFPVEMHISQRKVKKYSMKWNALKLLISVCLIVSLLAAIGSIVGLINSVKAYKPFHS

SEQ ID NO: 86 - DNA, 2535 bp, Brassica napus
  BnaA06g38000D
atgaagagctttgatacggtggaatccggtgacgccaccgggaataacttcgacgatgatggtcgggagaagagaacggggacgttgatgacagcgagtgcgcacataatcacggcggtggtaggctcaggagtgttgtcgttggcttgggctatagcacaacttggttgggtggcaggaatagtgattcttgtaacttttgccgtcatcaattactacacatccactatgctcgctgattgttatcggtcggacaccggaacacgcaattgtacttacatggacgtcgtccgagcttaccttggtatgtcatttattaatgtttagcatgtttgaaaatataaacaacatggtatagttcattggctttgaaagatttactttattttagttgtaaataacttaagattactaaaatcgaatgaagttagttagcattagtttgattttgaagatttttttttcgtcaaaagttgtaatcaattttttttatttctgtttcgggttgttttaatataatttgatttttaaaaaacaggtggtaagaaagtgcagttatgtggactggcacaatacgggtgtttcgtaggggtcactattggttacaccatcactgcatccataagcttagtgtaagttgcaacttgtgacttattttcgattacgttatggttactaacgttttgtgaagaaaaattaagtcttggttaatatgtcgtgttggctagagcgattgggaaagcaaattgttttcatgacaaaagcatggggcgaaatgttccatgccaaattatccattcatggcggtcttcgggatcgtggagattattcttagtcagattcccagctttcacaagctctcttttctctccattatcgccacagttatgtccttttcttatgcgtctatcggaattggtttagccatggccgttgtggcaagtacgcttttctctcccttttttttgtcaaaaaaatatacatttgtaaaacatgctcacgcaatttcaaaaccctatgcaaatatcttgacacatgcatttagttgtttagttttctgtttttacataaaaatttaggagctctgagatacgtctatacacatattcaaaactgattataaactgggttgacttgttcgattgcattagttgtcaatctttttaaatgccctcgtacatacaaaaagtttgtttaggcagaaaagcatctctatctaagcctcatatattgtaggattctcacagatttgtaagatgatatatgcatgtgatttttctaactaaaaaggtgtgatatgaaaggtgggaaggttggcaagacgggggctacgggcacagtggttggagtggacgtaaccacctctgacaaaatatggaagtcgtttcaagcgactggagacattgctttttcatacgcttattccagtattctcgttgagattcaggcatgcattaatcttttcttttatagatgttttggtggtcatcatcaaaatagctagtgtggcaatttttttatatcattattgctttttatttgcatttgagtttaaaaatcatatgatatatgttggtttggttaataacttttgatgatgttattaattatgattcttatcaagagaatatatatcttgaatttgacacagatcactttaaataataattagccttcaaccgcggttcatacgttaagctcttaaaaaacaatgaatctcaaacgttttctttctttgcttttttaggatacattgagatcaagcccgccagagaacaaagtcatgaagaaagcaagccttgccggagtctcaactacaacttttttctacatgttatgtggctgcattggatacgccgcatttggaaacaaagcccctggagacttccttaccgactttttttatgaaccttactggctcatcgattttgccaatgcttgcattgtcctccacctaatagcagcctatcaggtaaactcataagaatatagcttgagtctcagatcaaggattgctcgattataaagaaataattagtacctactactaacaactttgaagttaccgaagtctcaagatgaaaacaaacattgatgatgtttttaaaatgggcaggtgtttgcacaaccaatattccaatttgttgagaacaaatgcaacaaagcatggccagaaagcaatttcatcaccaaagaacattcgatgaacatactattcctcggaaaatgtcgcatcagcttcttcagactggtgtggaggacacttatgtgattttcacaacagttgtagcaatgatattccccttctttaacgcgatcttgggtcttatcggggcagccgcattctggccgctaacagtttacttcccggtggagatgcacatctcgcagagaaagattaagaagcattctatgagatggatagggttgaaactccttgtattggtttgtttgattgttaccctcctagccgcaataggatccatcgtcggcttgataaaaagtgtcaaggcatacaagcatttccacagttaa

SEQ ID NO: 87 - protein, 462 aa, Brassica napus
  BnaA06g38010D
MKSFDTVESGDATGNNFDDDGREKRTGTLMTASAHIITAVVGSGVLSLAWAIAQLGWVAGIVILVTFAVINYYTSTMLADCYRSDTGTRNCTYMDVVRAYLGGKKVQLCGLAQYGCFVGVTIGYTITASISLVAIGKANCFHDKRHGAKCSMPNYPFMAVFGIVEIILSQIPSFHKLSFLSIIATVMSFSYASIGIGLAMAVVASGKVGKTGATGTVVGVDVTTSDKIWKSFQATGDIAFSYAYSSILDTLRSSPPENKVMKKASLAGVSTTTFFYMLCGCIGYAAFGNKAPGDFLTDFFYEPYWLIDFANACIVLHLIAAYQVFAQPIFQFVENKCNKAWPESNFITKEHSMNILFLGKCRISFFRLVWRTAYVIFTTVVAMIFPFFNAILGLIGAAAFWPLTVYFPVEMHISQRKIKKHSMRWIGLKLLVLVCLIVTLLAAIGSIVGLIKSVKAYKHFHS

SEQ ID NO: 88 - DNA, 2535 bp, Brassica napus
  BnaA06g38010D
atgaagagctttgatacggtggaatccggtgacgccaccgggaataacttcgacgatgatggtcgggagaagagaacggggacgttgatgacagcgagtgcgcacataatcacggcggtggtaggctcaggagtgttgtcgttggcttgggctatagcacaacttggttgggtggcaggaatagtgattcttgtaacttttgccgtcatcaattactacacatccactatgctcgctgattgttatcggtcggacaccggaacacgcaattgtacttacatggacgtcgtccgagcttaccttggtatgtcatttattaatgtttagcatgtttgaaaatataaacaacatggtatagttcattggctttgaaagatttactttattttagttgtaaataacttaagattactaaaatcgaatgaagttagttagcattagtttgattttgaagatttttttttcgtcaaaagttgtaatcaattttttttatttctgtttcgggttgttttaatataatttgatttttaaaaaacaggtggtaagaaagtgcagttatgtggactggcacaatacgggtgtttcgtaggggtcactattggttacaccatcactgcatccataagcttagtgtaagttgcaacttgtgacttattttcgattacgttatggttactaacgttttgtgaagaaaaattaagtcttggttaatatgtcgtgttggctagagcgattgggaaagcaaattgttttcatgacaaaagacatggggcgaaatgttccatgccaaattatccattcatggcggtcttcgggatcgtggagattattcttagtcagattcccagctttcacaagctctcttttctctccattatcgccacagttatgtccttttcttatgcgtctatcggaattggtttagccatggccgttgtggcaagtacgcttttctctccctttttttgtcaaaaaaatatacatttgtaaaacatgctcacgcaatttcaaaaccctatgcaaatatcttgacacatgcatttagttgtttagttttctgtttttacataaaaatttaggagctctgagatacgtctatacacatattcaaaactgattataaactgggttgacttgttcgattgcattagttgtcaatctttttaaatgccctcgtacatacaaaaagtttgtttaggcagaaaagcatctctatctaagcctcatatattgtaggattctcacagatttgtaagatgatatatgcatgtgatttttctaactaaaaaggtgtgatatgaaaggtgggaaggttggcaagacgggggctacgggcacagtggttggagtggacgtaaccacctctgacaaaatatggaagtcgtttcaagcgactggagacattgctttttcatacgcttattccagtattctcgttgagattcaggcatgcattaatcttttcttttatagatgttttggtggtcatcatcaaaatagctagtgtggcaatttttttatatcattattgctttttatttgcatttgagtttaaaaatcatatgatatatgttggtttggttaataacttttgatgatgttattaattatgattcttatcaagagaatatatatcttgaatttgacacagatcactttaaataataattagccttcaaccgcggttcatacgttaagctcttaaaaaacaatgaatctcaaacgttttctttctttgcttttttaggatacattgagatcaagcccgccagagaacaaagtcatgaagaaagcaagccttgccggagtctcaactacaacttttttctacatgttatgtggctgcattggatacgccgcatttggaaacaaagcccctggagacttccttaccgactttttttatgaaccttactggctcatcgattttgccaatgcttgcattgtcctccacctaatagcagcctatcaggtaaactcataagaatatagcttgagtctcagatcaaggattgctcgattataaagaaataattagtacctactactaacaactttgaagtaccgaagtctcaagatgaaaacaaacattgatgatgtttttaaaatgggcaggtgtttgcacaaccaatattccaatttgttgagaacaaatgcaacaaagcatggccagaaagcaatttcatcaccaaagaacattcgatgaacatactattcctcggaaaatgtcgcatcagcttcttcagactggtgtggaggacagcttatgtgattttcacaacagttgtagcaatgatattccccttctttaacgcgatcttgggtcttatcggggcagccgcattctggccgctaacagtttacttcccggtggagatgcacatctcgcagagaaagattaagaagcattctatgagatggatagggttgaaactccttgtattggtttgtttgattgttaccctcctagccgcaataggatccatcgtcggcttgataaaaagtgtcaaggcatacaagcatttccacagttaa

SEQ ID NO: 89 - protein, 481 aa, Brassica napus
  BnaA09g57230D
MKSYATEYNPSAVETAGNNFDDDGREKRTGTLMTATAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHDKGHDAKCSVSNYPLMAAFGITQIVLSQIHNFHKLSFLSIIATVMSFSYASIGIGLALAALASGKVGKTDLTGTVVGVDVTASDKIWRSFQAAGDIAFSYAFSVVLVEIQACILSIRDDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFIAKDYSINIPLLGKCRINFFRLVWRSTYVILTTVVAMIFPFFNAILGLIGALTFWPLTVYFPVEMHISQRKVKKYTMRWIGLKLLVLVCLVVSLLAAVGSIVGLISSVKAYKPFHNLD

SEQ ID NO: 90 - DNA, 1446 bp, Brassica napus
  BnaA09g57230D
atgaaaagctacgccactgagtataatccctcggccgtggaaaccgccgggaataacttcgacgatgatggtcgggagaagagaacggggacgttgatgacggcgaccgcgcacataatcacggcggtgataggttctggagtcttgtcgttggcttgggctatagcacaacttggttgggtggcaggaacggtgattttggtaacttttgccgttataaattacttcacatctacaatgcttgcggactgctatcgatctccggacacaggaatacgtaattataattacatggacgttgtcagagcttaccttggtggttggaaagtgaagctgtgtggactggcacagtacgggagtctagtagggatcactattggctacaccatcactgcctccataagcttagtagcgatcgggaaagcaaattgttttcatgacaagggacatgatgcaaaatgttccgtatcaaattatccactcatggcggcgtttggtatcacccagattgttcttagtcagattcataattttcacaagctctcttttctctccattatcgctaccgttatgtccttctcttatgcatccatcggaattggcttagccttggctgctctggcaagtgggaaggttggtaagacggatctgacgggcacggtggttggagtagacgtaactgcgtctgacaaaatatggaggtcgtttcaagcagctggagacattgccttttcgtacgcattttccgttgttctcgttgagattcaggcatgcattctttcaattagagatgatacactgagatcaagcccaccagagaacaaagtcatgaaaaaagcaagccttgctggagtttcaactacaactggtttctacatcttgtgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaacttcatcgccaaagattattcgataaacataccattgctagggaaatgtcgcatcaacttcttcagattggtctggaggtcaacctatgtgattttgacaacagttgtagcgatgatattccccttctttaacgcgatcttgggccttattggagcactcacattctggccgctaacagtttacttcccagtggagatgcacatctcgcagagaaaggttaagaagtatactatgagatggatagggttgaaactccttgtattggtttgtttggttgtttcgctcctagctgcagtaggatccattgtcggcttgataagtagtgtaaaggcatacaagcctttccacaatttagattag

SEQ ID NO: 91 - protein, 1054 aa, Brassica napus
  BnaA09g57240D
MHRLYIDMSFTLHCLCFFSPLNMKTFDTSSAVESGTVAGNNVDDDGGEKRTGTLMTASAHIITAVIGSGVLSLAWAIAQLGWVAGTVLLVSFAVVVNYTSRMLADCYRSPDAGTRNNTYMDVVRAYLGGRKVQLCGLAQYGSLVGMTIGYTITASISLVAIGKANCFHDKGHGAKCLVSNYPAMAAFGIIQIVLSQIPNFHKLSFLSIIAAVMSFSYSSIGTGLALADLASGKVGKTELTGTVVGVDVTASDKLWKSFQAAGNIAFSYAYSVVLVEIQACIFSTRNDTLSSSPPENIVMKKASLVGVSTATAFYILCACMGYATFGSQAPGDLLTDFGFYEPYWLIDFANACIAVHLIGVYQQVIAQPIFQFVEKKCNKAWPESNFITKEHSMNIPLLGKCRINFFRLVWRTIYVIFSTVIAMIFPFFNAVLGLIGAVAFWPLTVYFPVEMHISQKKIKKYTMRWIGLKLLVLVCLIVSLLAAVGSIVGLISSLIRRKENMTLYISRLQFSHTHTHGPSTYPMINTNSYECLQNIISIDVCVHASSIYRYVIHSSSPMLLHISFLSSSVSPLKMKSFDTSSVVESGAGAGNNVDDDCREKRTGTLITASAHIITTVIGSGVLSLAWAIAQLGWVVGTVILVAFAVIVNYTSRMLADSYRSPEGTRNYTYMDVVRVYLGGRKVQLCGLAQFGSLVGVTIGYTITASISLVAIGKANCFHDKGHGAKCSVSNYPLMAAFGIVQIFLSQIPNFHKLSFLSIIATVMSFSYASIGFGLALAALASGKVGKTGLTGTVVGVDVTASDKLWKSFQAAGNIAFSYAYSVVLVEIQACIISINDDTLRSSPPENKVMKKASLAAVSTTTAFYILCGCIGYATFGNQAPGDFLTDFGFYEPYWLIDFANACIAVHLIGAYQQVFAQPIFQFVEKKCNQAWPESNFITKEHSMNIPLLGKCRINFFRLVWRTTYVIFSTVVAMIFPFFNAILGLIGAVAFWPLTVYFPVEMHISQKKVKKYSVRWIVLKLLVLVCLIVSLLAAIGSIVGLISSVKAYKPFHNLD

SEQ ID NO: 92 - DNA, 3076 bp, Brassica napus
  BnaA09g57240D
tcattcactcttcattgcctatgcttttgtatatctcattactctcttcctcagtttctcctctcaatatgaaaacctttgacacgagctcagcggttgaatccggtaccgtcgccgggaataacgtcgacgatgatggtggggagaagagaacggggacgttgatgacggcgagtgcgcacataatcacggctgtgataggttccggagtcttgtcgttggcttgggcaatagcacaacttggttgggtggcagggacagtgcttttggtaagctttgccgtcgtagttaattacacatccagaatgctcgccgactgttataggtctccggacgcaggaacacgcaataatacttacatggacgtcgttagagcttaccttggtatgaacatatgtcgttttgtgactctattactttggtattgttttaaccacaaaatagtatatttccaaaaagagctatatttcttttttttcttaaaaaaaatcatatactttcaaccctaaaaaggaatctgaaacaagtacaaaggccgattcaccaggtggctctaggttacaaggagagattaacaaacaacaacaatgaacagattaacaaacaacaacaatgatatatatttccttagcttagtctattttttattaaacaaaaacaaaaaaccataaaacgacaggcggtaggaaagtgcagctatgtggactagcacagtacgggagtctagtagggatgactattggttacaccatcactgcctccataagcttagtgtaagttacaacttgtgatttattttaatatataacctgttcgtttcacttacgtgacctatgactaaatgtcgttgctcgtgtgcatatgtcgcatgatcttgtgaccagttgcatgtattacagcgacatgcaaacggccataatgtcacatggttaccaacacgttaagagaatattaaagtcttgtttgatgttatgttggctagagcgattgggaaagcaaactgttttcatgacaagggacatggtgcgaaatgtttagtatcaaattatccagccatggcggcatttggaatcatccagattgttctaagtcagattcctaattttcacaagctctcttttctctccattatcgccgcggttatgtcattctcttattcatctatcggaactggcttagccttggccgatctggcaagtacgttccttcattcctatcttttctttcactttcaaaaatatacgtgtagagcattttcaagctatttctaaaccctatgcaaatatcttgatacatacatcaatattgtttaattttcagtagaaatcattctcattgatttgtaagatgatatatatttattacatatatgaatcttcaaactaatgactaaagcggtatgatgaaaggtgggaaggttggtaagacggaactgacaggcacggtggttggtgtggacgtaactgcgtctgacaagttatggaagtcattccaagcggctggaaacattgcattttcatacgcttattccgttgttctcgttgagattcaggcatgcatattttcaactagaaatgttggtggtcacactcggaatagtgtagcaatttttcccttttgaaacacattcttttatttgcatttatattttaattacatgatatatatgttggttggagtaatgacggttattagagcaccattaatcatagtattttagaaggtttatactaattaattaaaataaaaaggaatattgaaaaaaggagaagaacaacaaatagcaaagatacttcaagaaaaaatttgagaaacttttctatatgtgcaactcatttagtagttgagttgtttaaaagtaattaaagtatacttaataaaagtaaatattaatattttatttttgttgagaaacgctttttccttgttgatgatggtctatgtatgtgtaaaacaaaacgttattgggattcttatcatttttttttgacacagaactatattatcttcacttaattaaatacgtcttcaaccgtcgtttatggtggttgttttctgttgcaattcccttaaagtatatattgatgaaagagttaatatgacgtatgctagcccttaattaattaatgacagtattgctcatatatgcttgttttttattttttttgcaggatacactgagttcaagcccaccagagaacatagtcatgaaaaaagcaagtcttgtcggagtctcgactgcaactgctttctacatcttatgtgcttgcatgggatatgctacatttggaagccaagcccctggagacctccttactgactttggtttttatgaaccttattggctcatcgattttgccaatgttgcatcgctgtccacctaatcggagtctatcaggtagctcactaaaacaaaaaaagtttaagatagataattagtatctatcactcacaatttttaagtttaagtataaggctcaagaaaagataaacaaaaacattgatgatgttaagacaacgagcaggtgattgcacaaccaattttccagtttgttgagaagaaatgcaacaaagcatggccagagagcaatttcatcaccaaagaacattcgatgaacataccattgcttggaaaatgtcgcatcaacttcttcagactggtatggaggacaatctatgtgatattctcaacagttatagcaatgatattccccttcttcaacgcggtcttgggccttatcggggcagtcgcattctggccgctaacagtttacttcccagtggagatgcacatctcgcagaaaaagattaagaagtatactatgagatggatagggttgaaactccttgtattggtttgtttgattgtttcgctcctagctgcagtaggatccatcgtcggcttgataagtagtgtcaaggcatacaagcctttccacaatttggattagttacttgtaacctaagctagttgaatacatagttaataacgaactatgtacaatgagtatggaagaacaaacgaatacatagttaatgacgagctatgtttttttttttttgtcatcatgacgag

SEQ ID NO: 93 - protein, 507 aa, Brassica napus
  BnaC01g42990D
MKGFNTEQDHPAAESGNVYDVSDPTKNVDDDGREKRTGTWLTASAHIITAVIGSGVLSLAWAIAQLGWIAGTLILVIFSFITYFTSTMLADCYRAPDPVTGKRNYTYMDVVRSYLGGRKVQLCGVAQYGNLIGITVGYTITASISLVAVGKANCFHKKGHEADCTISNYPYMAVFGIIQIILSQIPNFHKLSFLSLMAAVMSFTYATIGIGLAIATVAGGKVGKTNMTGTVVGVDVTAAQKIWRSFQAVGDIAFAYAYATVLIEIQASFHIKYLWNLVSFEYEPLDRIVDTLKSSPAENKSMKRASLVGVSTTTFFYILCGCLGYAAFGNKAPGDFLTDFGFYEPFWLIDFANACIAFHLIGAYQVFAQPIFQFVEKRCNRNWPDNKFITSEYSVNVLFLGKFNISLFRLVWRTAYVVITTVVAMIFPFFNAILGLIGAASFWPLTVYFPVEMHIAQTKVKKYSPRWIGLKMLCWVCLIVSLLAAAGSIAGLISSVKTYKPFRTIHE

SEQ ID NO: 94 - DNA, 3995 bp, Brassica napus; identical to SEQ ID NO: 84
  BnaC01g42990D

SEQ ID NO: 95 - protein, 356 aa, Brassica napus
  BnaC05g07760D
YGNLVGVSIGYTITASISLVAIGKANCFHGKGHGAKCTASNYPYMGAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNIAPGDFLTDFGFYEPFWLVIFANVCIAVHLVGAYQVYVQPFFQFVESKCNKKWPESNFINKEYSLKIPLLGKFRVNHFRLVWRTNYVILTTFIAMIFPFFNSILGLLGALAFWPLTVYFPVAMHIAQTKVKKYSGRWLALHLLVLVCLIVSALAAVGSIVGLINNVKKYKPFESID

SEQ ID NO: 96 - DNA, 1071 bp, Brassica napus
  BnaC05g07760D
tacggcaacctcgttggggtctctattggttacaccatcactgcctccataagcttagtagcgattgggaaagcaaattgttttcatggtaagggacatggtgcgaaatgtaccgcatcgaattatccatacatgggggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacgctgacaggtacggtgataggagtggacgtatctgcgtctgataaagtatggaaagcgtttcaagcggttggggatattgcgttttcgtacgcttacaccactattctcattgagatccaggacacattgagatcaagcccaccagagaacaaagtgatgaagaaagcaagtcttattggagtctcaaccacaactgttttctacctcttatgtggttgcattgggtatgctgcattcggaaacatagcccctggtgacttccttaccgactttgggttttacgaacctttctggctcgtcattttcgccaatgtttgcattgctgtccatttagtaggtgcctatcaggtatatgttcagccctttttccaatttgttgagagcaaatgcaacaaaaagtggcctgaaagcaatttcatcaacaaagagtactcgttgaagataccattgctcggaaaatttcgtgtcaaccacttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgcacctcctcgtgttggtttgcttgattgtctccgccttagctgcagtgggatccattgttggcctaatcaataatgtcaagaaatacaagcctttcgagagtatagactaa

SEQ ID NO: 97 - protein, 469 aa, Brassica napus
  BnaC05g49200D
MKSFDAVHNPSAVESADANVDDDGREKRTGTLMTASAHIITAVIGSGVLSLAWAIAQLGWVAGTLILVTFAIVNYYTSTMLADCYRSDAGARNYTYMDVVRSYLGGRKVQLCGLAQYGCLVGITIGYTITASISLVAIWKATCFHKKGHGAKCSIPNYPFMAAFGVVEIFLSQLPNFHKLSFLSIIAAVMSFSYASIGIGLAIAVVASGKVGKTGVTGTVVGVDVTASDKIWKAFQATGDIAFSYSFSTILVEIQDTLRSSPPENKVMKKATLAGVSTTTVFYILCGCMGYAAFGNRAPGDFLTDFGFYEPYWLINFANACIVLHLIAAYQVFAQPIFQLVENKCNKAWPENNFINKEHSINIPFLGKWRINFFRLVWRTAYVILTTFVAVIFPFFNSILGLIGATAFWPLTVYFPVEMHISQRKKEFMYGPNPNFKGSRTPTPSIQQRGDTGSGNSGAAVMITVLDQF

SEQ ID NO: 98 - DNA, 1410 bp, Brassica napus
  BnaC05g49200D
atgaaaagctttgacgcggtgcataatccctctgcggtggaatccgctgacgccaacgtcgacgatgatggtcgggagaagagaacggggacgttgatgacggcgagtgcgcacataatcacggcggtgataggttccggagtgttgtcgttggcctgggctatagcacagcttggttgggtggcaggaacactgattcttgtaacttttgccatcgtcaattactacacatccactatgctcgccgactgttatagatcggacgcaggagctcgcaactatacgtacatggacgtcgtccgatcttaccttggtggtaggaaagtgcagttatgtggactggcacaatacgggtgtctcgtagggatcactattggttacaccatcactgcctctataagtttagtagcgatttggaaagcaacttgttttcataaaaaaggacatggtgcgaaatgttccatcccaaattatccattcatggcggccttcggggtcgtggagatttttcttagtcagcttcctaattttcacaagctctcttttctctccattatcgccgccgttatgtcattctcttatgcgtctatcggaattggtttagccattgccgttgtggcaagtggaaaggttggtaagacgggtgtgacgggcacggtggttggagtggacgtgaccgcatctgacaaaatatggaaggcgtttcaagcaactggagacattgcattttcatactctttttccactattctcgttgagattcaggatacattgagatcaagcccaccagaaaacaaagtcatgaaaaaagcaacactcgccggagtctcaacgacaactgttttctacatcttatgtggctgcatgggatatgctgcatttggaaaccgagcccccggagacttccttactgactttggtttttatgaaccttactggctcatcaactttgccaatgcttgcatcgtcctccacctaatcgcagcctatcaggtgtttgcacaaccaattttccaacttgttgagaacaaatgcaacaaagcatggccagaaaacaatttcatcaacaaagaacattcgataaacataccattcctcggaaaatggcgcatcaacttcttcagactggtgtggaggacagcatatgtgattttgacaacatttgttgcagtgatattccccttcttcaactcgatcttgggccttatcggagcaacagcgttctggccgctaacagtttacttcccagtggagatgcacatctcgcagagaaagaaggagttcatgtatggtccaaatcctaacttcaaaggctctagaactccaacaccgtctattcaacaacgaggagacactgggagtggcaactccggtgctgctgtgatgatcacggttctagatcagttttga

SEQ ID NO: 99 - protein, 482 aa, Brassica napus
  BnaC05g49210D
MKSFDTVESGDATGNNFDDDGREKRTGTLVTASAHIITAVVGSGVLSLAWAIAQLGWVAGIVILVTFAVINYYTSTMLADCYRSDTGTRNCTYMDVVRAYLGGRKVQLCGLAQYGCFVGVTIGYTITASISLVAIGKANCFHDKGHGAKCSMPNYPFMAAFGIVEIILSQIPSFHKLSFLSIIATVMSFSYASIGIGLAMAVVASGKVGKTGVTGTVAGVDVTASDKIWKSFQATGDIAFSYAYSSILVEIQACILSSIDVLGVIIKIDTLRSSPPENKVMKKASLAGVSTTTFFYMLCGCIGYAAFGNKAPGDFLTEFFYEPYWLIDYANACIVLHLIAAYQVFAQPIFQFVENKCNKAWPESNFITIEHSMNIPFLGKCRVNFFRLVWRTAYVILTTVVAMIFPFFNSILGLIGAAAFWPLTVYFPVEMHISQRKIKKYSMRWIGLKLLVSVCLIVTLLAAIGSIVGLIKSVKAYKHFHS

SEQ ID NO: 100 - DNA, 2537 bp, Brassica napus
  BnaC05g49210D
atgaagagctttgatacggtggaatccggtgacgccaccgggaataacttcgacgatgatggtcgggagaagagaacggggacgttgatgacagcgagtgcgcacataatcacggcggtggtaggctcaggagtgttgtcgttggcttgggctatagcacaacttggttgggtggcaggaatagtgattcttgtaacttttgccgtcatcaattactacacatccactatgctcgctgattgttatcggtcggacaccggaacacgcaattgtacttacatggacgtcgtccgagcttaccttggtatgtcatttattaatgtttagcatgtttgaaaatataaacaacatggtatagttcattggctttgaaagatttactttattttagttgtaaataacttaagattactaaaatcgaatgaagttagttagcattagtttgattttgaagatttttttttcgtcaaaagttgtaatcaattttttttatttctgtttcgggttgttttaatataatttgatttttaaaaaacaggtggtaagaaagtgcagttatgtggactggcacaatacgggtgtttcgtaggggtcactattggttacaccatcactgcatccataagcttagtgtaagttgcaacttgtgacttattttcgattacgttatggttactaacgttttgtgaagaaaaattaagtcttggttaatatgtcgtgttggctagagcgattgggaaagcaaattgttttcatgacaaaagacatggggcgaaatgttccatgccaaattatccattcatggcggtcttcgggatcgtggagattattcttagtcagattcccagctttcacaagctctcttttctctccattatcgccacagttatgtccttttcttatgcgtctatcggaattggtttagccatggccgttgtggcaagtacgcttttctctcccttttttttgtcaaaaaaatatacatttgtaaaacatgctcacgcaatttcaaaaccctatgcaaatatcttgacacatgcatttagttgtttagttttctgtttttacataaaaatttaggagctctgagatacgtctatacacatattcaaaactgattataaactgggttgacttgttcgattgcattagttgtcaatctttttaaatgccctcgtacatacaaaaagtttgtttaggcagaaaagcatctctatctaagcctcatatattgtaggattctcacagatttgtaagatgatatatgcatgtgatttttctaactaaaaaggtgtgatatgaaaggtgggaaggttggcaagacgggggctacgggcacagtggttggagtggacgtaaccacctctgacaaaatatggaagtcgtttcaagcgactggagacattgctttttcatacgcttattccagtattctcgttgagattcaggcatgcattaatcttttcttttatagatgttttggtggtcatcatcaaaatagctagtgtggcaatttttttatatcattattgctttttatttgcatttgagtttaaaaatcatatgatatatgttggtttggttaataacttttgatgatgttattaattatgattcttatcaagagaatatatatcttgaatttgacacagatcactttaaataataattagccttcaaccgcggttcatacgttaagctcttaaaaaacaatgaatctcaaacgttttctttctttgcttttttaggatacattgagatcaagcccgccagagaacaaagtcatgaagaaagcaagccttgccggagtctcaactacaacttttttctacatgttatgtggctgcattggatacgccgcatttggaaacaaagcccctggagacttccttaccgactttttttatgaaccttactggctcatcgattttgccaatgcttgcattgtcctccacctaatagcagcctatcaggtaaactcataagaatatagcttgagtctcagatcaaggattgctcgattataaagaaataattagtacctactactaacaactttgaagttaccgaagtctcaagatgaaaacaaacattgatgatgtttttaaaatgggcaggtgtttgcacaaccaatattccaatttgttgagaacaaatgcaacaaagcatggccagaaagcaatttcatcaccaaagaacattcgatgaacatactattcctcggaaaatgtcgcatcagcttcttcagactggtgtggaggacagcttatgtgattttcacaacagttgtagcaatgatattccccttctttaacgcgatcttgggtcttatcggggcagccgcattctggccgctaacagtttacttcccggtggagatgcacatctcgcagagaaagattaagaagcattctatgagatggatagggttgaaactccttgtattggtttgtttgattgttaccctcctagccgcaataggatccatcgtcggcttgataaaaagtgtcaaggcatacaagcatttccacagttaa

SEQ ID NO: 101 - protein, 481 aa, Brassica napus
  BnaC08g42410D
MKSFDTSSVVESGAGAGNNVDDDCREKRTGTLITASAHIITTVIGSGVLSLAWAIAQLGWVVGTVILVAFAVIVNYTSRMLADSYRSPEGTRNYTYMDVVRVYLGGRKVQLCGLAQFGSLVGVTIGYTITASISLVAIGKANCFHDKGHGAKCSVSNYPLMAAFGIVQIFLSQIPNFHKLSFLSIIATVMSFSYASIGFGLALAALASGKVGKTGLTGTVVGVDVTASDKLWKSFQAAGNIAFSYAYSVVLVEIQACILSINDDTLRSSPPENKVMKKASLAAVSTTTAFYILCGCIGYATFGNQAPGDFLTDFGFYEPYWLIDFANACIAVHLIGAYQQVFAQPIFQFVEKKCNQAWPESNFITKEHSMNVPLLGKCRINFFRLVWRTTYVIFSTVVAMIFPFFNAILGLIGAVAFWPLTVYFPVEMHISQKKVKKYSVRWIVLKLLVFVCLIVSLLAAIGSIVGLISSVKAYKPFHNLD

SEQ ID NO: 102 - DNA, 3178 bp, Brassica napus
  BnaC08g42410D
ctcttcatcgcctatgcttttgcatatctcttttctctcttcttcagtttctcctctcaaaatgaaaagcttcgacacgagctcagtggttgaatccggtgctggcgccgggaataacgtcgacgatgattgtcgggagaagagaacggggaccttgataacggcgagtgcccacataatcacgacagtgataggttctggagtcttgtcgttggcttgggctatagcacaacttggttgggtggtaggaacagtgattttggtagcctttgccgtcatagttaattacacatccagaatgctcgccgacagttatcgatccccggagggaacacgcaactatacttacatggacgtcgtccgagtctaccttggtatgaatatgttcatttcttaactgaactactactttggtagtaaacaaaatgttgttttgtcttagcttagtctatctattactaacaaaacaaaaagaaaaaaacataaaacgacaggtggtaggaaagtgcagctgtgtggactagcacagttcgggagtctcgtaggggttactattggttacaccatcactgcctccataagcttagtgtaagttacaacttgtgattattttgatatataaaacatgtttgtttcaatttctaacgtgacctacgactaagtattgctcacatggccataatgtcatatggttaccaatatgttaagaaaatatttaagtcttgtttgatgttattttggctagggcgattgggaaagcaaattgttttcatgacaagggacatggtgcgaaatgttccgtatcaaattatccactcatggcggcgtttggaatcgtccagatttttcttagtcagattcctaattttcacaagctctcttttctctccattatcgccaccgttatgtccttctcttatgcatctatcggttttggcttagccttggccgctctggcaagtacgttctcttcttcctatataatttgactttcaaaatatatttgtagaacattctcaagctatttctaaaccctatgcaaatatcttgatacatacttaatacatttatattttagttttcagtacaaatctttctttttcagtagaaatcatcctcattgatttgtaagatgatatatattaactatttacacatctattgtttaaaaaaaaaaaatttatacatatatgaatcttcaaactaatggctaaagcggtacgatgaaaggtgggaaggttggtaagacgggactgacaggcacggtggttggagtggatgtaactgcgtctgacaaattatggaagtcatttcaagcggctggaaacattgccttttcatacgcttattccgttgttctcgttgagattcaggcatgtattatttcaattaatgatgtcagtacactcagaaatcagaatagtgtagcattttctaatattacagtgaaacttctataaattaataatgttgggactacatcaaaactataatttttttattaatttatagagatactaatttatcgatatactaatagaaccaaaaactcaatttgaaactataaaattatattattttatagatttttagtatatattaatttatagattattaatttaaagaggttatactgtagtttttttattcttttatttacatgatatatattttggttggaataatgactgttattagagcaccattagtcatagtattagtatcgtagggggtgtctaataattaaaataaaaaggaatattgaaaaagagaagaacagaaaatagcaaaaaacgattcttgttgacatacttcaagaaaaaaaagtccgatttttttacaagtgtaactcatttattagttgggttgtttaaaagtaattaaagtatacttgtaaaactaaatatttttggcaccgaactatactattattttcactacaatacgtcttcaaccgtcgtttatggtggttgttttcttgttgtaattcccttaaagtatatattgatggatgattagagttaatatgatgtatgttagctcttaaagacaatgttgctaatatatgcttgtttttttccttgcaggacacactgagatcaagcccaccagagaacaaagtcatgaaaaaagcaagccttgctgcagtctcaactacaactgctttctacatcttatgtggctgcatcggatatgctacatttggaaaccaagcccccggagacttccttactgactttggtttttatgaaccttactggctcatcgattttgctaatgcttgcatcgctgtccaccttatcggagcttatcaggtaactcataaaaaaggagaaatactttagaatattactaaaacagcttactattctaaattaacacacgcaaaatgatcaaaataacattaactaaaatttaaaaatatacttttattttatagttgggtttaggtttagtgaatagagtttaggggttagtatttaaaaagtggaagtgcagagtttgaaatgttttttgtcattttctccttatgtgataattttgtcataatattttttttgtggtatctaagtcatttgtcctaaaaaagtcaagagtttaatataatgagcaggtgtttgcacaaccaatattccagtttgttgagaagaaatgcaatcaggcgtggccagaaagcaacttcatcaccaaagaacattcgatgaacataccgttgcttggaaaatgtcgcattaacttcttcagactggtgtggaggacaacctatgtgattttctcaacagttgtagcaatgatatttcccttcttcaacgctatcttgggccttattggggcagtcgcattctggccgctaacagtttacttcccggtggagatgcacatctcgcagaaaaaggttaagaagtattctgtgagatggatagtattgaaactccttgttttggtttgtttaattgtttcgctcctagctgccataggatccatcgttggcttgataagtagtgtcaaggcatacaagcctttccacaatttagattagttacttgtaacctaagctatctgaatacatagttaataatgagttatgttagcaatgagtttatggatgaaccgatatatagtacttatttctgaaaaaaggttatatagtacttcattttacgatcacatttaggac

SEQ ID NO: 103 - protein, 504 aa, Brassica napus
  BnaC08g42420D
MHRLYIDMSFTLHCLCFFSPLNMKSFDTSSAVESGTVAGNNVDDDGGEKRTGTLMTASAHIITAVIGSGVLSLAWAIAQLGWVAGTVLLVSFAVVVNYTSRMLADCYRSPDAGTRNNTYMDVVRAYLGGRKVQLCGLAQYGSLVGMTIGYTITASISFVAIGKANCFHDKGHGAKFSVSNYPAMAAFGIIQIVLSQIPNFHKLSFLSIIAAVMSFSYSSIGTGLALADLASGKVGKTELTGTVVGVDVTASDKLWKSFQAAGNIAFSYAYSVVLVEIQACIFSTRNDTLSSSPPENIVMKKASIVGVSTATAFYILCACMGYATFGSQAPGDLLTDFGFYEPYWLIDFANACIAVHLIGAYQQVIAQPIFQFVEKKCNKAWPESNFITKEHSMNIPLLGKCRINFFRLVWRTIYVIFSTVIAMIFPFFNAVLGLIGAVAFWPLTVYFPVEMHISQKKIKKYTMRWIGLKLLVLVCLIVSLLAAVGSIVGLISSVKAYKPFHNLD

SEQ ID NO: 104 - DNA, 3077 bp, Brassica napus
  BnaC08g42420D
tcattcactcttcattgcctatgcttttgtatatctcattactctcttcctcagtttctcctctcaatatgaaaacctttgacacgagctcagcggttgaatccggtaccgtcgccgggaataacgtcgacgatgatggtggggagaagagaacggggacgttgatgacggcgagtgcgcacataatcacggctgtgataggttccggagtcttgtcgttggcttgggcaatagcacaacttggttgggtggcagggacagtgcttttggtaagctttgccgtcgtagttaattacacatccagaatgctcgccgactgttataggtctccggacgcaggaacacgcaataatacttacatggacgtcgttagagcttaccttggtatgaacatatgtcgttttgtgactctattactttggtattgttttaaccacaaaatagtatatttccaaaaagagctatatttcttttttttcttaaaaaaaatcatatactttcaaccctaaaaaggaatctgaaacaagtacaaaggccgattcaccaggtggctctaggttacaaggagagattaacaaacaacaacaatgaacagattaacaaacaacaacaatgatatatatttccttagcttagtctattttttattaaacaaaaacaaaaaaccataaaacgacaggcggtaggaaagtgcagctatgtggactagcacagtacgggagtctagtagggatgactattggttacaccatcactgcctccataagcttagtgtaagttacaacttgtgatttattttaatatataacctgttcgtttcacttacgtgacctatgactaaatgtcgttgctcgtgtgcatatgtcgcatgatcttgtgaccagttgcatgtattacagcgacatgcaaacggccataatgtcacatggttaccaacacgttaagagaatattaaagtcttgtttgatgttatgttggctagagcgattgggaaagcaaactgttttcatgacaagggacatggtgcgaaatgtttagtatcaaattatccagccatggcggcatttggaatcatccagattgttctaagtcagattcctaattttcacaagctctcttttctctccattatcgccgcggttatgtcattctcttattcatctatcggaactggcttagccttggccgatctggcaagtacgttccttcattcctatcttttctttcactttcaaaaatatacgtgtagagcattttcaagctatttctaaaccctatgcaaatatcttgatacatacatcaatattgtttaattttcagtagaaatcattctcattgatttgtaagatgatatatatttattacatatatgaatcttcaaactaatgactaaagcggtatgatgaaaggtgggaaggttggtaagacggaactgacaggcacggtggttggtgtggacgtaactgcgtctgacaagttatggaagtcattccaagcggctggaaacattgcattttcatacgcttattccgttgttctcgttgagattcaggcatgcatattttcaactagaaatgttggtggtcacactcggaatagtgtagcaatttttcccttttgaaacacattcttttatttgcatttatattttaattacatgatatatatgttggttggagtaatgacggttattagagcaccattaatcatagtattttagaaggtttatactaattaattaaaataaaaaggaatattgaaaaaaggagaagaacaacaaatagcaaagatacttcaagaaaaaatttgagaaacttttctatatgtgcaactcatttagtagttgagttgtttaaaagtaattaaagtatacttaataaaagtaaatattaatattttatttttgttgagaaacgctttttccttgttgatgatggtctatgtatgtgtaaaacaaaacgttattgggattcttatcatttttttttgacacagaactatattatcttcacttaattaaatacgtcttcaaccgtcgtttatggtggttgttttctgttgcaattcccttaaagtatatattgatgaaagagttaatatgacgtatgctagcccttaattaattaatgacagtattgctcatatatgcttgttttttattttttttgcaggatacactgagttcaagcccaccagagaacatagtcatgaaaaaagcaagtcttgtcggagtctcgactgcaactgctttctacatcttatgtgcttgcatgggatatgctacatttggaagccaagcccctggagacctccttactgactttggtttttatgaaccttattggctcatcgattttgccaatgcttgcatcgctgtccacctaatcggagtctatcaggtagctcactaaaacaaaaaaagtttaagatagataattagtatctatcactcacaatttttaagtttaagtataaggctcaagaaaagataaacaaaaacattgatgatgttaagacaacgagcaggtgattgcacaaccaattttccagtttgttgagaagaaatgcaacaaagcatggccagagagcaatttcatcaccaaagaacattcgatgaacataccattgcttggaaaatgtcgcatcaacttcttcagactggtatggaggacaatctatgtgatattctcaacagttatagcaatgatattccccttcttcaacgcggtcttgggccttatcggggcagtcgcattctggccgctaacagtttacttcccagtggagatgcacatctcgcagaaaaagattaagaagtatactatgagatggatagggttgaaactccttgtattggtttgtttgattgtttcgctcctagctgcagtaggatccatcgtcggcttgataagtagtgtcaaggcatacaagcctttccacaatttggattagttacttgtaacctaagctagttgaatacatagttaataacgaactatgtacaatgagtatggaagaacaaacgaatacatagttaatgacgagctatgtttttttttttttgtcatcatgacgag

SEQ ID NO: 105 - protein, 481 aa, Brassica napus
  BnaC08g42430D
MKSFHTEYNPSAVEAAGNNFDDDGREKRTGTVMTASAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHEKGHGAKCSVSNYPLMAAFGIIQIVLSQIHNFHKLSFLSIIATVMSFSYASIGIGLALAALASGKVGKTDLTGTVVGVDVTASDKIWRSFQAAGDIAFSYAFSVVLVEIQACILSIRDDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFITKDYSINIPLLGKCRINFFRLVWRSTYVILTTVAAMIFPFFNAILGLIGALTFWPLTVYFPVEMHISQKKVKKYTMRWIGLKLLVLVCLVVSLLAAVGSIVGLISSVKAYKPFHNLD

SEQ ID NO: 106 - DNA, 1446 bp, Brassica napus
  BnaC08g42430D
atgaaaagcttccacactgagtataatccctcggccgtggaagccgccgggaataacttcgacgacgatggtcgggagaagagaacggggacggtgatgacggcaagtgctcacattatcactgctgtgataggttccggagtcttgtccttggcttgggctatagcacaacttggttgggtggcaggaacagtgattttggtaacttttgccgttataaattacttcacatctacaatgcttgccgactgttatcgatctccggacacaggaatacgtaattataattacatggacgttgtcagagcttaccttggtggttggaaagtgaagctatgtggtctggcacagtacgggagtctagtagggatcactattggttacaccatcactgcctccataagcttagtagcgatagggaaagcaaattgttttcatgaaaagggacatggtgcaaaatgttccgtatcgaattatccactcatggcggcgtttggtatcatccagattgttcttagtcagattcataattttcacaagctctcttttctctccattatcgccaccgttatgtccttctcttatgcatccatcggaattggcttggccttggccgctctggcaagtgggaaggttggtaagacggatctgacgggcacggtggttggagtagacgtaactgcgtctgacaaaatatggaggtcgtttcaagcagctggagacattgccttttcgtacgcattttccgttgttctcgttgagattcaggcatgcattctttcaattagagatgatacactgagatcaagcccaccagagaacaaagtcatgaaaaaagcaagccttgctggagtttcaactacaactggtttctacatcttatgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaactttatcaccaaagattattcgataaacataccattgctagggaaatgtcgcatcaacttcttcagattggtctggaggtcaacctatgtgattttgacaacagttgcagcaatgatattccccttcttcaacgcgatcttgggccttatcggagcactcacattctggccgctaacagtttacttcccagtggagatgcacatctcgcagaaaaaggttaagaagtatactatgagatggatagggttgaaactccttgtattggtttgtttggttgtttcgctcctagctgcagtaggatccattgtcggcctcataagtagtgtaaaggcatacaagcctttccacaatttagattag

SEQ ID NO: 107 - protein, 479 aa, Brassica napus
  BnaCnng14480D
MEKKSMFIEQSFTDHKSGDMNKNFDDDGRQKRTGTWMTGSAHIITAVIGSGVLSLAWAIAQLGWVAGPAVLMAFSFITYFTSTMLADCYRSPDPVTGKRNYTYMEVVRSYLGGRKVMLCGLAQYGNLIGITIGYTITASISMVAVKRSNCFHKNGHNVKCSTSNTPFMIIFACIQIVLSQIPNFHNLSWLSILAAVMSFSYASIGVGLSIAKVAGGGVHARTALTGVTVGVDVTGSDKVWRTFQAVGDIAFAYAYSTDTLKASPPSENKAMKRASLVGVSTTTFFYMLCGCVGYAAFGNNAPGNFLTGFGFYEPFWLIDFANVCIAVHLVGAYQVFCQPIFQFVESQSAKRWPDNKFITGEYKMNVPCGGDFGISLFRLVWRTSYVVVTAVVAMIFPFFNDFLGLIGAASFWPLTVYFPIEMHIAQKKMKKFSFTWTWLKILSWACFLVSLVAAAGSVQGLIQSLLNADLFTKSVAPFS

SEQ ID NO: 108 - DNA, 2742 bp, Brassica napus
  BnaCnng14480D
atgatttatatatagaagaaaataacttatataggtttattgatgctatggaaaaaataggaggaagaaaagtgatgttatgtggattggctcaatacgggaatctgattggaataacaatcggctacacaatcacagcgtcgattagcatggtgtaagctttcattctttctttcagaatgatagtcaataatagcaggctctcctttttcacctattttcacccacgtttattatgttaggacaggtgactaactcttttttataattattaattttaccttttaaagaacagatgctatgataggtaagagatatgatatataatatctacaaaagtttttctttgtcacaagttatttgatatgtacagagtaatataaatttaaattctattgagtgtgggagtcgaaaggagctcaaattttcaaagtgaaaagttagatctagtaggatcgttgagattttgtattctaaatttatcaaattttttttgtctggaactttatatatttataattattaaggaatgggttttaaagtacgaaagaaagaaaaaaattaaaatgacaaaattaagaattcatgaaaacagggcagtgaagaggtcgaattgtttccacaagaatggacataatgtgaaatgttccacttcaaacactcccttcatgatcatattcgcatgcatccaaattgtacttagccaaatcccaaatttccacaatctctcttggctctccattctagcggccgtaatgtccttttcttacgcctccattggtgtcggtctctccatcgccaaagtggccggtaaataaatatacgttcttatccttataataatttcttcttttaaaagtgagtcctaatggttttgaagaataaacaaaggtggcggtgtgcacgcaaggacggccctgacaggagttacggtcggagttgatgttactggttctgataaagtatggagaacattccaagcggttggagatatcgcatttgcttatgcctactcaaccgtacttattgagatacaggcaagtagtcacctttaacgtactgctattttgtactcaccagtttaatttaaaatgttttttaaacatctcgcacacctgttaagaaaggagtattagtttttcactataacccttattaaatgtttcagctaatacaaatggtatctttggaaaaaataataatatactcagacctgaatatactacatatttttataatttaatataacggaaaatatggttataattgaaagttgaaacttatgttaaaactttgtattggcaactctaaaactaaactcagttttaaaaaaattagccataaactgacacttctgaaatggaaggattatgtttagagctgagttttagagaccgcgataagagagagccaaaaaaaaattaaacgtgtgtcggtttgtgacaaagtaaaggccacttcagatgaattattatttgtagtaaaacatgagaggaaaccagagtctaactagtaggcttttattcaataaataaatacttataaaatgatttaactttgagtacggtttacaactgatgttttggcttcttttgtacacaaaagattaatattctaactttaattaatgtatttcattttaaaagtaaaaaaaaatctaaaaatatggattgtagaattttattggaaaaaaacaaaactaacaaaaactaattaatcaaaaatgactcttgacttagttaattttatacttttatatttatcactaaataacattaaagtcaccaattacgtattgtcatttcagataattgtaaacgatttagtgaactacattttgtgtgtgttttgatctaccactactaaagtatgtacaactgttcatctctaaaccatggcaggacaccttaaaagcaagtccaccatcagaaaacaaagccatgaaaagagcaagccttgtgggtgtatccacaacgaccttcttttacatgttatgcgggtgtgtgggttatgctgcctttggcaataatgcgcctggaaatttcctaaccggttttggtttttatgagcccttctggctaatcgactttgcgaatgtctgcatcgctgtgcatctagttggggcctatcaggtcttttgccagccaatcttccaatttgtagagagccagagtgcaaaacgttggccagataacaagtttattacaggagagtacaaaatgaacgtcccttgcggtggtgattttggtatcagcttgtttagattggtttggaggacttcatatgttgtagttacggctgttgtagcaatgatcttccctttcttcaacgatttcttgggtcttattggagcagcttccttttggcctttgactgtttactttcccattgagatgcatattgctcagaaaaagatgaagaaattttctttcacttggacatggctgaaaatcttgagctgggcttgtttcctcgtctccctcgttgctgctgctggatccgtgcaaggactcatacaaagtcttaaggatttcaagcctttccaggctcccgagtagaactgtgttttgtctaatttcttcaagaacacaactcccttgcactctcttttctccaataatagtgtatgttcgtttgtaatatatttgtgttcttgtttcattacgaaatgttgaacctatatttgctgtctgaaacaaatctatcatcaataagtccctctttcttttctttgtccttggt

SEQ ID NO: 109 - protein, 479 aa, Brassica napus
  BnaCnng25620D
MKSFNTDQHGHSAAESADVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATDTLRSSPAENKAMKRASFVGVSTTTFFYILCGCLGYAAFGNKAPGDFLTDFGFYEPFWLIDFANACIAFHLIGAYQVFAQPIFQFVEKKCNRNWPDNKFITSEYSVNIPFLGKFSINLFRLVWRTAYVVITTLVAMIFPFFNAILGLIGAASFWPLTVYFPVEMHIAQTKVKKYSSRWIGLKMLCWVCLIVSLLAAAGSIAGLISSVKTYKPFRTIHE

SEQ ID NO: 110 - DNA, 3994 bp, Brassica napus
  BnaCnng25620D
ctacaattagacttcttcgtttactctctctctctctctctctgacttacacatttacttatcttcttctctctgtcaacctcctttgctctcttctctcactatgaagagtttcaacaccgatcaacacggtcactccgcggcggaatccggcgacgtctacgccatgtccgatccaacaaagaacgttgatgacgatggccgagagaagaggacggggacgtggcttacggcgagtgcgcatattatcacggcggtaataggctcaggagtgttgtcgttggcatgggccatagctcagcttggttggatagcagggactttgattctgatcattttctcgtttatcacgtacttcacttctacgatgctcgctgattgctaccgtgcgccggatcccctcaccggaaaacgaaactacacttacatggacgttgttcgatcttacctcggtatggttttttctttgtttttttttttttaactttttactgtaaagaagattgaaaaatagacactttttagaccaacccttttttgaaaagggttctataccaaaaattcaattttgttaagtaatttttaaggcatggttggttccttttccaaaaaaaaagagagaggataacataaggttctctttcattaaaataatatactgattttgtctggtatttagttaggctgatccttgtctggaaaactattgtggtcagaataggaaaataattattagtattaaaaatttatactaatattaattaaaaatgacaaaatatatattagtaataaaaacatataatataatttataagcgacagtaacttttaggtcaatttgaaataaaactggttatgtaatgagtttatatagaacaatgatggtggtttataaaatagttgatgttacgaactataagatcaatcataagaaatcatcattgatatcttttgaaacaatcgaaaagcttatgcatccgatgagttgtggttaggaatgtagataaagtaatggatttatagattttctaacatttcctgccaatttggttttacagaagaaaaaaaatctatcgggcatataaattactgttgcgtataaattgattttttttgtagacgcttataaattgatatatatccttttaaatatttaaatttaactgaatataaaaacagtggtaaccgttcttttaattttctagttagaaaaaatgttgttgaaataattaaaggcttggttattatttatacatggattatattcgtaagcaatttaaagtttgttgtctctagtttaataaacgatgattttttacgaattctttttattaagtaaaaacactgagttattgacaaaaaaaaagagtaaaacaactgagttcaatgtaaagtgtggtaaccgtccttttaattttctaatgataaactatggttgttgaaaaattaattagacttcgctattattttcataaggattacatacttcaaataatttagagtatgttgtctagttcaataaacaatgaggttttacttcaaatcattttaagtaaaacattgaactgaacgcaaaagtgtggtaaccgtcctttaatttccaactatacaacggttgttgaaataattaattaggctgaaatttttcttaaataactcacggttttgtactgtttataagatccaaaactgtgcaacccgaggaaaacagaaattaaataatagtttgacatatgaggcaaagggttttggtagtattagcattattgttggtatttgatgttgatggagtattatatatactagtatagttccgtggagtgttttatgtttgatgtatgaaacagaagattaattaatagcttaaaaacaaatgtaataaataatcatagactgaaataatacattggaattattgaaaattgacaggtggtaggaaagtgcagctttgtggagtggcacagtatgggaatctgataggaatcactgttgggtacaccatcactgcttctattagtttggtgtaagtcaactatgtgtgaatatgtgattttactttatgaccgttaccaaatgtacagtttagttatctaccatattgatgtgatcagtatggtttcttgcgcagagcgatcgggaaagcgaactgttaccacaataaagggcaccatgcagattgtacaatatcgaactatccatatatggcggccttcgggatcattcagatccttcttagccagatccccaactttcacaagctctcttttctctcccttatggctgcggttatgtctttcgcttacgcaagtattgggattggccttgccatcgcgacggtcgcaggtacatatacaaatctttgaagatttaattagttgcaaaacaacaactactaacaattattggtcatcaagttgtagttttcaaagtacctatatcaataattgtaacaagatagataatacaataaagtaacggttgatatgttacgatatgaaaaggtgggaaagtgggtaagacgaatatgacgggaacggtggtaggagttgatgtaactgcggctcagaagatatggagatcgtttcaagcggttggagacatagcgtttgcatatgcttacgccacggttctcattgagattcaggcaagttaaccatcaatcaattttactattgagtcttgtttgctttgaatatgaacctcttaatagaattgtggtttgaaattattgacaatgacaatcaattctctatggaccacttttaatacaaagggtaaaaagaagagaatctgtcttttagcttaaaggtataacatgtgcttattagtgacaagatgtcacattcaaagacagcaaacaatgatatcaatggactttagcttaatgagttgacaatatagttaaaattttgttgtctcttaatgatattagcgttcacctttctagtgtacatgcatttagttcaatagagtgtatatgtcgactagaaagtgacggcttaataagatttaagttaaacacatgagacaaaactggatttgcacacactaaccggtgaaaatttgatactttttgcaggacacattgagatctagccccgcagagaacaaagctatgaaaagagcaagttttgtgggagtatcaaccaccactttcttctacatcttatgtggttgtcttggatatgctgcatttggaaacaaagcccctggtgatttcctcacagatttcggattctacgagccattttggctcattgactttgcaaacgcttgcattgctttccatctcattggtgcctatcaggtaaaaccaaatccaaaaggagaaaaagattgttttctttttgcactttctcgcagtttagctatgaaaaagaaacatacttcttgcaggtgttcgcgcagcccatattccagtttgttgagaagaaatgcaatagaaactggcctgacaacaagttcatcacatctgaatattcagtaaacataccattccttggaaaattcaacatcaacctcttcagactagtgtggaggacagcttatgtggttataacaactttagtagctatgatattccctttcttcaacgccatcttgggtcttatcggagcagcttccttctggcctttaactgtttatttccccgtggagatgcacatagcacaaactaaggttaagaaatactctcctagatggattgggctgaaaatgttgtgctgggtttgcttgatcgtctccctgttagctgctgctggatccatcgctggactgataagtagtgtgaagacatacaagcccttccggactatccatgagtgagtttgagatccatgcatgagagtgaaaaaatgtaacagtttggtattttctttgtaactgtttggtgtctaaaatccaaatgaaaatattgtattgcttaaaaagctacatgaatgtctatgtatcatccacaactttcagtgtaatacatatgacccaataatagcttt

SEQ ID NO: 111 - protein, 485 aa, Brassica napus
  CAD92449 AAP1
MKSFNTDQHGHSAAESGDVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTNFGFYEPFWLIDFANACIAFHLIGAYQVFAQPIFQFVEKKCNRNWPDNKFITSEYSVNIPFLGKFSINLFRLVWRTAYVVITTLVAMIFPFFNAILGLIGAASFWPLTVYFPVEMHIAQTKVKKYSSRWIGLKMLCWVCLIVSLLAAAGSIAGLISSVKTYKPFRTIHE

SEQ ID NO: 112 - DNA, 3994 bp, Brassica napus
  CAD92449 AAP1
ctacaattagacttcttcgtttactctctctctctctctctctgacttacacatttacttatcttcttctctctgtcaacctcctttgctctcttctctcactatgaagagtttcaacaccgatcaacacggtcactccgcggcggaatccggcgacgtctacgccatgtccgatccaacaaagaacgttgatgacgatggccgagagaagaggacggggacgtggcttacggcgagtgcgcatattatcacggcggtaataggctcaggagtgttgtcgttggcatgggccatagctcagcttggttggatagcagggactttgattctgatcattttctcgtttatcacgtacttcacttctacgatgctcgctgattgctaccgtgcgccggatcccctcaccggaaaacgaaactacacttacatggacgttgttcgatcttacctcggtatggttttttctttgtttttttttttttaactttttactgtaaagaagattgaaaaatagacactttttagaccaacccttttttgaaaagggttctataccaaaaattcaattttgttaagtaatttttaaggcatggttggttccttttccaaaaaaaaagagagaggataacataaggttctctttcattaaaataatatactgattttgtctggtatttagttaggctgatccttgtctggaaaactattgtggtcagaataggaaaataattattagtattaaaaatttatactaatattaattaaaatgacaaaatatatattagtaataaaaacatataatataatttataagcgacagtaacttttaggtcaatttgaaataaaactggttatgtaatgagtttatatagaacaatgatggtggtttataaaatagttgatgttacgaactataagatcaatcataagaaatcatcattgatatcttttgaaacaatcgaaaagcttatgcatccgatgagttgtggttaggaatgtagataaagtaatggatttatagattttctaacatttcctgccaatttggttttacagaagaaaaaaaatctatcgggcatataaattactgttgcgtataaattgattttttttgtagacgcttataaattgatatatatccttttaaatatttaaatttaactgaatataaaaacagtggtaaccgttcttttaattttctagttagaaaaaatgttgttgaaataattaaaggcttggttattatttatacatggattatattcgtaagcaatttaaagtttgttgtctctagtttaataaacgatgattttttacgaattctttttattaagtaaaaacactgagttattgacaaaaaaaaagagtaaaacaactgagttcaatgtaaagtgtggtaaccgtccttttaattttctaatgataaactatggttgttgaaaaattaattagacttcgctattattttcataaggattacatacttcaaataatttagagtatgttgtctagttcaataaacaatgaggttttacttcaaatcattttaagtaaaacattgaactgaacgcaaaagtgtggtaaccgtcctttaatttccaactatacaacggttgttgaaataattaattaggctgaaatttttcttaaataactcacggttttgtactgtttataagatccaaaactgtgcaacccgaggaaaacagaaattaaataatagtttgacatatgaggcaaagggttttggtagtattagcattattgttggtatttgatgttgatggagtattatatatactagtatagttccgtggagtgttttatgtttgatgtatgaaacagaagattaattaatagcttaaaaacaaatgtaataaataatcatagactgaaataatacattggaattattgaaaattgacaggtggtaggaaagtgcagctttgtggagtggcacagtatgggaatctgataggaatcactgttgggtacaccatcactgcttctattagtttggtgtaagtcaactatgtgtgaatatgtgattttactttatgaccgttaccaaatgtacagtttagttatctaccatattgatgtgatcagtatggtttcttgcgcagagcgatcgggaaagcgaactgttaccacaataaagggcaccatgcagattgtacaatatcgaactatccatatatggcggccttcgggatcattcagatccttcttagccagatccccaactttcacaagctctcttttctctcccttatggctgcggttatgtctttcgcttacgcaagtattgggattggccttgccatcgcgacggtcgcaggtacatatacaaatctttgaagatttaattagttgcaaaacaacaactactaacaattattggtcatcaagttgtagttttcaaagtacctatatcaataattgtaacaagatagataatacaataaagtaacggttgatatgttacgatatgaaaaggtgggaaagtgggtaagacgaatatgacgggaacggtggtaggagttgatgtaactgcggctcagaagatatggagatcgtttcaagcggttggagacatagcgtttgcatatgcttacgccacggttctcattgagattcaggcaagttaaccatcaatcaattttactattgagtcttgtttgctttgaatatgaacctcttaatagaattgtggtttgaaattattgacaatgacaatcaattctctatggaccacttttaatacaaagggtaaaaagaagagaatctgtcttttagcttaaaggtataacatgtgcttattagtgacaagatgtcacattcaaagacagcaaacaatgatatcaatggactttagcttaatgagttgacaatatagttaaaattttgttgtctcttaatgatattagcgttcacctttctagtgtacatgcatttagttcaatagagtgtatatgtcgactagaaagtgacggcttaataagatttaagttaaacacatgagacaaaactggatttgcacacactaaccggtgaaaatttgatactttttgcaggacacattgagatctagccccgcagagaacaaagctatgaaaagagcaagttttgtgggagtatcaaccaccactttcttctacatcttatgtggttgtcttggatatgctgcatttggaaacaaagcccctggtgatttcctcacagatttcggattctacgagccattttggctcattgactttgcaaacgcttgcattgctttccatctcattggtgcctatcaggtaaaaccaaatccaaaaggagaaaaagattgttttctttttgcactttctcgcagtttagctaatgaaaaagaaacatacttcttgcaggtgttcgcgcagcccatattccagtttgttgagaagaaatgcaatagaaactggcctgacaacaagttcatcacatctgaatattcagtaaacataccattccttggaaaattcaacatcaacctcttcagactagtgtggaggacagcttatgtggttataacaactttagtagctatgatattccctttcttcaacgccatcttgggtcttatcggagcagcttccttctggcctttaactgtttatttccccgtggagatgcacatagcacaaactaaggttaagaaatactctcctagatggattgggctgaaaatgttgtgctgggtttgcttgatcgtctccctgttagctgctgctggatccatcgctggactgataagtagtgtgaagacatacaagcccttccggactatccatgagtgagtttgagatccatgcatgagagtgaaaaaatgtaacagtttggtattttctttgtaactgtttggtgtctaaaatccaaatgaaaatattgtattgcttaaaaagctacatgaatgtctatgtatcatccacaactttcagtgtaatacatatgacccaataatagcttt

SEQ ID NO: 113 - protein, 478 aa, Brassica napus
  XP_013645981 AAP8-like
MKSLDTLHNPSAVESGNAAVKNVDDDGREKRTGTFLTASAHIITAVIGSGVLSLAWALAQLGWVAGTMILVIFAIITYYTSTLLADCYRAPDPITRTRNYTYMGVVRAYLGGKKVQLCGLAQYGNLVGVSIGYTITASISLVAIGKANCFHGKGHGAKCTASNYPYMGAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNLSPGDFLTDFGFYEPFWLVIFANVCIAVHLVGAYQVYVQPFFQFVESKCNKKWPESNFINKEYSLKIPLLGKFRVNFFRLVWRTNYVILTTFIAMIFPFFNSILGLLGALAFWPLTVYFPVAMHIAQTKVKKYSGRWLALNLLVLVCLIVSALAAVGSIVGLINNVKKYKPFESID

SEQ ID NO: 114 - DNA, 1437 bp, Brassica napus
  XP_013645981 AAP8-like
atgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacctctactttgctcgccgattgctacagagcgccggaccccatcaccagaacacgcaactacacgtacatgggcgtcgttcgagcttaccttggtggtaaaaaggtgcagctatgtggactagcacagtacggcaacctcgttggggtctctattggttacaccatcactgcctccataagcttagtagcgattgggaaagcaaattgttttcatggtaagggacatggtgcgaaatgtaccgcatcgaattatccatacatgggggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacactgacaggtacggtgataggagtggacgtatctgcgtctgataaagtgtggaaagcgtttcaagcggttggggatattgcgttttcgtacgcttacaccactattctcattgagatacaggacacattgagatcaagcccaccagagaacaaagtgatgaagaaagcaagtcttattggagtctcaaccacaactgttttctacctcttatgtggttgcattgggtatgctgcattcggaaacttatcccctggtgacttccttaccgactttgggttttacgaacctttctggctcgtcattttcgccaatgtttgcattgctgtccatttagtaggtgcctatcaggtatatgttcagccttttttccagtttgttgagagcaaatgtaacaaaaagtggcctgaaagcaatttcatcaacaaagaatactcgttgaagataccattgctcggaaaatttcgtgtcaacttcttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgctggtttgcttgattgtctccgccctagctgctgtgggatccattgttggcctaatcaataatgtcaagaaatacaagcctttcgagagtatagactaa

SEQ ID NO: 115 - protein, 515 aa, Brassica napus
  XP_013661681 AAP1 X1
MKSFNTDQHGHSAAESGDVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTDFGFYEPFWLIDFANACIAFHLIGAYQVKPNPKGEKDCFLFALSRSLANEKETYFLQVFAQPIFQFVEKKCNRNWPDNKFITSEYSVNIPFLGKFNINLFRLVWRTAYVVITTLVAMIFPFFNAILGLIGAASFWPLTVYFPVEMHIAQTKVKKYSPRWIGLKMLCWVCLIVSLLAAAGSIAGLISSVKTYKPFRTIHE

SEQ ID NO: 116 - DNA, 3995 bp, Brassica napus; identical to SEQ ID NO: 84
  XP_013661681 AAP1 X1

SEQ ID NO: 117 - protein, 485 aa, Brassica napus
  XP_013661682 AAP1 X2
MKSFNTDQHGHSAAESGDVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTDFGFYEPFWLIDFANACIAFHLIGAYQVFAQPIFQFVEKKCNRNWPDNKFITSEYSVNIPFLGKFNINLFRLVWRTAYVVITTLVAMIFPFFNAILGLIGAASFWPLTVYFPVEMHIAQTKVKKYSPRWIGLKMLCWVCLIVSLLAAAGSIAGLISSVKTYKPFRTIHE

SEQ ID NO: 118 - DNA, 3992 bp, Brassica napus
  XP_013661682 AAP1 X2
ctacaattagacttcttcgtttactctctctctctctctctctgacttacacatttacttatcttcttctctctgtcaacctcctttgctctcttctctcactatgaagagtttcaacaccgatcaacacggtcactccgcggcggaatccggcgacgtctacgccatgtccgatccaacaaagaacgttgatgacgatggccgagagaagaggacggggacgtggcttacggcgagtgcgcatattatcacggcggtaataggctcaggagtgttgtcgttggcatgggccatagctcagcttggttggatagcagggactttgattctgatcattttctcgtttatcacgtacttcacttctacgatgctcgctgattgctaccgtgcgccggatcccctcaccggaaaacgaaactacacttacatggacgttgttcgatcttacctcggtatggttttttctttgtttttttttttttaactttttactgtaaagaagattgaaaaatagacactttttagaccaacccttttttgaaaagggttctataccaaaaattcaattttgttaagtaatttttaaggcatggttggttccttttccaaaaaaaaagagagaggataacataaggttctctttcattaaaataatatactgattttgtctggtatttagttaggctgatccttgtctggaaaactattgtggtcagaataggaaaataattattagtattaaaaatttatactaatattaattaaaaatgacaaaatatatattagtaataaaaacatataatataatttataagcgacagtaacttttaggtcaatttgaaataaaactggttatgtaatgagtttatatagaacaatgatggtggtttataaaatagttgatgttacgaactataagatcaatcataagaaatcatcattgatatcttttgaaacaatcgaaaagcttatgcatccgatgagttgtggttaggaatgtagataaagtaatggatttatagattttctaacatttcctgccaatttggttttacagaagaaaaaaaatctatcgggcatataaattactgttgcgtataaattgattttttttgtagacgcttataaattgatatatatccttttaaatatttaaatttaactgaatataaaaacagtggtaaccgttcttttaattttctagttagaaaaaatgttgttgaaataattaaaggcttggttattatttatacatggattatattcgtaagcaatttaaagtttgttgtctctagtttaataaacgatgattttttacgaattctttttattaagtaaaaacactgagttattgacaaaaaaaaagagtaaaacaactgagttcaatgtaaagtgtggtaaccgtccttttaattttctaatgataaactatggttgttgaaaaattaattagacttcgctattattttcataaggattacatacttcaaataatttagagtatgttgtctagttcaataaacaatgaggttttacttcaaatcattttaagtaaaacattgaactgaacgcaaaagtgtggtaaccgtctttaatttccaactatacaacggttgttgaaataattaattaggctgaaatttttcttaaataactcacggttttgtactgtttataagatccaaaactgtgcaacccgaggaaaacagaaattaaataatagtttgacatatgaggcaaagggttttggtagtattagcattattgttggtatttgatgttgatggagtattatatatactagtatagttccgtggagtgttttatgtttgatgtatgaaacagaagattaattaatagcttaaaaacaaatgtaataaataatcatagactgaaataatacattggaattattgaaaattgacaggtggtaggaaagtgcagctttgtggagtggcacagtatgggaatctgataggaatcactgttgggtacaccatcactgcttctattagtttggtgtaagtcaactatgtgtgaatatgtgattttactttatgaccgttaccaaatgtacagtttagttatctaccatattgatgtgatcagtatggtttcttgcgcagagcgatcgggaaagcgaactgttaccacaataaagggcaccatgcagattgtacaatatcgaactatccatatatggcggccttcgggatcattcagatccttcttagccagatccccaactttcacaagctctcttttctctcccttatggctgcggttatgtctttcgcttacgcaagtattgggattggccttgccatcgcgacggtcgcaggtacatatacaaatctttgaagatttaattagttgcaaaacaacaactactaacaattattggtcatcaagttgtagttttcaaagtacctatatcaataattgtaacaagatagataatacaataaagtaacggttgatatgttacgatatgaaaaggtgggaaagtgggtaagacgaatatgacgggaacggtggtaggagttgatgtaactgcggctcagaagatatggagatcgtttcaagcggttggagacatagcgtttgcatatgcttacgccacggttctcattgagattcaggcaagttaaccatcaatcaattttactattgagtcttgtttgctttgaatatgaacctcttaatagaattgtggtttgaaattattgacaatgacaatcaattctctatggaccacttttaatacaaagggtaaaaagaagagaatctgtcttttagcttaaaggtataacatgtgcttattagtgacaagatgtcacattcaaagacagcaaacaatgatatcaatggactttagcttaatgagttgacaatatagttaaaattttgttgtctcttaatgatattagcgttcacctttctagtgtacatgcatttagttcaatagagtgtatatgtcgactagaaagtgacggcttaataagatttaagttaaacacatgagacaaaactggatttgcacacactaaccgggaaaatttgatactttttgcaggacacattgagatctagccccgcagagaacaaagctatgaaaagagcaagttttgtgggagtatcaaccaccactttcttctacatcttagtggttgtcttggatatgctgcatttggaaacaaagcccctggtgatttcctcacagatttcggattctacgagccattttggctcattgactttgcaaacgcttgcattgctttccatctcattggtgcctatcaggtaaaaccaaatccaaaaggagaaaaagattgttttctttttgcactttctcgcagtttagctaatgaaaaagaaacatacttcttgcaggtgttcgcgcagcccatattccagtttgttgagaagaaatgcaatagaaactggcctgacaacaagttcatcacatctgaatattcagtaaacataccattccttggaaaattcaacatcaacctcttcagactagtgtggaggacagcttatgtggttataacaactttagtagctatgatattccctttcttcaacgccatcttgggtcttatcggagcagcttccttctggcctttaactgtttatttccccgtggagatgcacatagcacaaactaaggttaagaaatactctcctagatggattgggctgaaaatgttgtgctgggtttgcttgatcgtctccctgttagctgctgctggatccatcgctggactgataagtagtgtgaagacatacaagcccttccggactatccatgagtgagtttgagatccatgcatgagagtgaaaaaatgtaacagtttggtattttctttgtaactgtttggtgtctaaaatccaaatgaaaatattgtattgcttaaaaagctacatgaatgtctatgtatcatccacaactttcagtgtaatacatatgacccaataatagcttt

SEQ ID NO: 119 - protein, 485 aa, Brassica napus
  XP_013661683 AAP1 X3
MKSFNTDQHGHSAAESGDVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTDFGFYEPFWLIDFANACIAFHLIGAYQVFAQPIFQFVEKKCNRNWPDNKFITSEYSVNIPFLGKFNINLFRLVWRTAYVVITTLVAMIFPFFNAILGLIGAASFWPLTVYFPVEMHIAQTKVKKYSSRWIGLKMLCWVCLIVSLLAAAGSIAGLISSVKTYKPFRTIHE

SEQ ID NO: 120 - DNA, 3995 bp, Brassica napus; identical to SEQ ID NO: 84
  XP_013661683 AAP1 X3

SEQ ID NO: 121 - protein, 481 aa, Brassica napus
  XP_013676681 AAP6
MEKKSMFIEQSFTDHKSGDMNKNFDDDGRQKRTGTWMTGSAHIITAVIGSGVLSLAWAIAQLGWVAGPAVLMAFSFITYFTSTMLADCYRSPDPVTGKRNYTYMEVVRSYLGGRKVMLCGLAQYGNLIGITIGYTITASISMVAVKRSNCFHKNGHNVKCSTSNTPFMIIFACIQIVLSQIPNFHNLSWLSILAAVMSFSYASIGIGLSIAKVAGGGVHARTALTGVTVGVDVTGSDKVWRTFQAVGDIAFAYAYSTVLIEIQDTLKASPPSENKAMKRASLVGVSTTTFFYMLCGCVGYAAFGNNAPGNFLTGFGFYEPFWLIDFANVCIAVHLVGAYQVFCQPIFQFVESQSAKRWPDNKFITGEYKMNVPCGGDFGISLFRLVWRTSYVVVTAVVAMIFPFFNDFLGLIGAASFWPLTVYFPIEMHIAQKKMKKFSFTWTWLKILSWACFLVSLVAAAGSVQGLIQSLKDFKPFQAPE

SEQ ID NO: 122 - DNA, 2742 bp, Brassica napus; identical to SEQ ID NO: 108
  XP_013676681 AAP6

SEQ ID NO: 123 - protein, 486 aa, Brassica napus
  XP_013696427; XP_013640943; XP_013716098; AAP8
MSPSPPPTMKSLDTLHNPSAVESGNAAVKNVDDDGREKRTGTFLTASAHIITAVIGSGVLSLAWALAQLGWVAGTMILVIFAIITYYTSTLLADCYRAPDPITRTRNYTYMGVVRAYLGGKKVQLCGLAQYGNLVGVSIGYTITASISLVAIGKANCFHGKGHGAKCTASNYPYMGAFGGLQILLSQIPNFHKLSFLSIIAAVMSFSYASIGIGLAIAKVASGKVGKTTLTGTVIGVDVSASDKVWKVFQAVGDIAFSYAYTTILIEIQDTLRSSPPENKVMKKASLIGVSTTTVFYLLCGCIGYAAFGNIAPGDFLTDFGFYEPFWLVIFANVCIAVHLVGAYQVYVQPFFQFVESKCNKKWPESNFINKEYSLKIPLLGKFRVNHFRLVWRTNYVILTTFIAMIFPFFNSILGLLGALAFWPLTVYFPVAMHIAQTKVKKYSGRWLALNLLVLVCLIVSALAAVGSIVGLINNVKKYKPFESID

SEQ ID NO: 124 - DNA, 1461 bp, Brassica napus
  XP_013696427; XP_013640943; XP_013716098; AAP8
atgtctccctctccccctcctacaatgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacctctactttgctcgccgattgctacagagcgccggaccccatcaccagaacacgcaactacacgtacatgggcgtcgttcgagcttaccttggtggtaaaaaggtgcagctatgtggactagcacagtacggcaacctcgttggggtctctattggttacaccatcactgcctccataagcttagtagcgattgggaaagcaaattgttttcatggtaagggacatggtgcgaaatgtaccgcatcgaattatccatacatgggggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacactgacaggtacggtgataggagtggacgtatctgcgtctgataaagtgtggaaagtgtttcaagcggttggggatattgcgttttcgtacgcttacaccactattctcattgagatccaggacacattgagatcaagcccaccagagaacaaagtgatgaagaaagcaagtcttattggagtctcaaccacaactgttttctacctcttatgtggttgcattgggtatgctgcattcggaaacatagcccctggtgacttccttaccgactttgggttttacgaacctttctggctcgtcattttcgccaatgtttgcattgctgtccatttagtaggtgcctatcaggtatacgttcagccctttttccaatttgttgagagcaaatgcaacaaaaagtggcctgaaagcaatttcatcaacaaagagtactcgttgaagataccattgctcggaaaatttcgtgtcaaccacttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgttggtttgcttgattgtctccgccttagcggcagtgggatccattgttggcctaatcaataatgtcaagaaatacaagcctttcgagagtatagactaa

SEQ ID NO: 125 - protein, 515 aa, Brassica napus
  XP_013723586 AAP1-like X1
MKSFNTDQHGHSAAESADVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTDFGFYEPFWLIDFANACIAFHLIGAYQVKPNPKGEKDCFLFALSRSLANEKETYFLQVFAQPIFQFVEKKCNRNWPDNKFITSEYSVNIPFLGKFSINLFRLVWRTAYVVITTLVAMIFPFFNAILGLIGAASFWPLTVYFPVEMHIAQTKVKKYSSRWIGLKMLCWVCLIVSLLAAAGSIAGLISSVKTYKPFRTIHE

SEQ ID NO: 126 - DNA, 3995 bp, Brassica napus; identical to SEQ ID NO: 84
  XP_013723586 AAP1-like X1

SEQ ID NO: 127 - protein, 485 aa, Brassica napus
  XP_013723587 AAP1-like isoform X2
MKSFNTDQHGHSAAESADVYAMSDPTKNVDDDGREKRTGTWLTASAHIITAVIGSGVLSLAWAIAQLGWIAGTLILIIFSFITYFTSTMLADCYRAPDPLTGKRNYTYMDVVRSYLGGRKVQLCGVAQYGNLIGITVGYTITASISLVAIGKANCYHNKGHHADCTISNYPYMAAFGIIQILLSQIPNFHKLSFLSLMAAVMSFAYASIGIGLAIATVAGGKVGKTNMTGTVVGVDVTAAQKIWRSFQAVGDIAFAYAYATVLIEIQDTLRSSPAENKAMKRASFVGVSTTTFFYILCGCLGYAAFGNKAPGDFLTDFGFYEPFWLIDFANACIAFHLIGAYQVFAQPIFQFVEKKCNRNWPDNKFITSEYSVNIPFLGKFSINLFRLVWRTAYVVITTLVAMIFPFFNAILGLIGAASFWPLTVYFPVEMHIAQTKVKKYSSRWIGLKMLCWVCLIVSLLAAAGSIAGLISSVKTYKPFRTIHE

SEQ ID NO: 128 - DNA, 3995 bp, Brassica napus; identical to SEQ ID NO: 84
  XP_013723587 AAP1-like isoform X2

SEQ ID NO: 129 - protein, 484 aa, Brassica napus
  XP_013748815 AAP1-like
MKGFNTEQDHPAAESGNVYDVSDPTKNVDDDGREKRTGTWLTASAHIITAVIGSGVLSLAWAIAQLGWIAGTLILVIFSFITYFTSTMLADCYRAPDPVTGKRNYTYMDVVRSYLGGRKVQLCGVAQYGNLIGITVGYTITASISLVAVGKANCFHKKGHEADCTISNYPYMAVFGIIQIILSQIPNFHKLSFLSLMAAVMSFTYATIGIGLAIATVAGGKVGKTNMTGTVVGVDVTAAQKIWRSFQAVGDIAFAYAYATVLIEIQDTLKSSPAENKSMKRASLVGVSTTTFFYILCGCLGYAAFGNKAPGDFLTDFGFYEPFWLIDFANACIAFHLIGAYQVFAQPIFQFVEKRCNRNWPDNKFITSEYSVNVPFLGKFNISLFRLVWRTAYVVITTVVAMIFPFFNAILGLIGAASFWPLTVYFPVEMHIAQTKVKKYSPRWIGLKMLCWVCLIVSLLAAAGSIAGLISSVKTYKPFRTIHE

SEQ ID NO: 130 - DNA, 3995 bp, Brassica napus; identical to SEQ ID NO: 84
  XP_013748815 AAP1-like

SEQ ID NO: 131 - protein, 483 aa, Hordeum vulgare subsp. vulgare
  BAJ85485
MGMEKSKANPAAFSIAEAGFGDRTDIDDDGRERRTGTLVTASAHIITAVIGSGVLSLAWAIAQLGWVIGPAVLVAFSVITWFCSSLLADCYRSPDPVHGKRNYTYGQAVRANLGVSKYRLCSLAQYLNLVGVTIGYTITTAISMGAIGRSNCFHRNGHNAACEASNTTNMIIFAAIQILLSQLPNFHKVWWLSIVAAVMSLAYSSIGLGLSIAKIAGGVHAKTTLTGVTVGVDVSASEKIWRTFQSLGDIAFAYSYSNVLIEIQDTLRSSPAENTVMKKASLIGVSTTTTFYMLCGVLGYAAFGSSAPGNFLTGFGFYEPFWLVDVGNVCIVVHLVGAYQVFCQPFYQFVEGWARSRWPDSAFLHAERVVQLPAIVGGGEFPVSPFRLVWRTAYVALTAVVAMLFPFFNDFLGLIGAVSFWPLTVYFPVEMYMAQAKVRRFSPTWTWMNVLSIACLVVSVLAAAGSVQGLVKDVAGYKPFKVS

SEQ ID NO: 132 - DNA, 1452 bp, Hordeum vulgare subsp. vulgare
  BAJ85485
atggggatggagaagagcaaggctaaccctgccgccttcagcatcgctgaggccggctttggagaccggacggacatcgacgacgacggccgcgagaggcgtaccggtacgctggtgacggccagcgcacacatcatcacggcggtgatcgggtccggggtgctgtcgctggcgtgggccatcgcacagctcgggtgggtcatcggccccgcggtgctcgtcgccttctccgtcatcacctggttctgctccagcctactggccgactgctatcgctcgccggaccccgtccacggcaagcgcaactacacctacggccaagccgtcagggcaaacctaggagttagcaagtacaggctctgctcactggcccaatacctcaacttggttggcgtgaccattggctacaccatcaccacggccatcagcatgggggcgatcggacggtccaattgcttccaccgcaatggccacaatgcggcctgcgaggcatccaacaccaccaacatgattatatttgctgccatccaaatcttgctctcgcagctccccaacttccacaaggtctggtggctctccattgttgctgccgtcatgtccctcgcctactcgtccattggtctcggcctctccatagcaaaaatcgcaggtggggtgcatgccaagacaacgctaacaggggtgaccgttggggtggatgtatctgcgagtgagaaaatttggagaacgttccagtctcttggggacatcgcctttgcatactcctactccaatgttctcatcgaaatccaggacacgctgcggtcgagcccggcggagaacacggtgatgaagaaggcatccttgatcggcgtttccacgaccaccacgttctacatgctgtgcggggtgctgggctacgcggcgttcggcagcagcgccccgggtaacttcctcacgggcttcggcttctacgagcccttctggctcgtcgacgtcggcaacgtctgcatcgtcgtgcacctcgtcggcgcctaccaggtcttctgccagcccttctaccagttcgtcgagggctgggcgcgctcccggtggcccgacagcgccttcctccacgccgagcgagtcgtgcaactcccggccattgtcggcggcggcgagttccccgtgagcccatttcgcctggtctggcgaacggcgtacgtggccctcacggcggtggtggccatgttgttccccttcttcaacgactttcttggcctcatcggcgccgtctcgttttggccgctcaccgtctacttccccgttgagatgtacatggcacaggccaaggtgcggcggttctcgccgacgtggacgtggatgaacgtgcttagcatcgcctgccttgtcgtctctgtcctcgcagccgctggttcggtgcaggggctcgtcaaggacgtggcagggtacaagccattcaaggtctcctaa

SEQ ID NO: 133 - protein, 490 aa, Hordeum vulgare subsp. vulgare
  BAJ91439.1 predicted protein
MTKDVEMAARNGSKGAAAGEAYYPSPPGQGGDVDVDDDGKQRRTGTVWTASAHIITAVIGSGVLSLAWATAQLGWVVGPVTLMLFAAITYYTSGLLADCYRTGDPLTGKRNYTYMDAVASYLSRWQVWACGVFQYVNLVGTAIGYTITASISAAAINKANCFHKNGRAADCGVYDSMYMVVFGVVQIFFSQVPNFHDLWWLSILAAVMSFTYASIAVGLSLAQTISGPTGKSTLTGTEVGVDVDSAQKIWLAFQALGDIAFAYSYSMILIEIQDTVRSPPAENKTMKKATLVGVSTTTAFYMLCGCLGYAAFGNGAKGNILTGFGFYEPYWLIDFANVCIVVHLVGAYQVFCQPIFAAVENFAAATWPNAGFITREHRVAAGKRLGFNLNLFRLTWRTAFVMVSTLLAILMPFFNDILGFLGAIGFWPLTVYFPVEMYIRQRGIQRYTTRWVALQTLSFLCFLVSLAAAVASIEGVTESLKNYVPFKTKS

SEQ ID NO: 134 - DNA, 1491 bp, Hordeum vulgare subsp. vulgare; identical to SEQ ID NO: 26
  BAJ91439.1 predicted protein

SEQ ID NO: 135 - protein, 737 aa, Triticum urartu
  EMS56484
MEVVTALTNVEVPATGTVAEATDRSDAERASKWARCWRILGWTLGEGIVGEDFGWSWGGGAGGCFYFPYFTCGQGSGDDDCVRGGAWGRGFGAGASPMTTAFHSAARGGAGGGLGQVAPAILSPDMPVALGLGVGHLSEGHGSPQPPAPVTLVDPLRDSARGFTREEVVAFGGIPDPVSAGRWMSARIQELPEVDDMQQRCAMREAKLHDAEISTGYFSSHGSDPFVVATHSDGGQRAFGYWIYPLGDASQLEAMGMEKGKADPATFSIAEAGFGDRTDIDDDGRERRTGTLVTASAHIITAVIGSGVLSLAWAIAQLGWVIGPAVLVAFSVITWFCSSLLADCYRSPDPVHGKRNYTYGQAVRANLGVSKYRLCSLAQYVNLVGVTIGYTITTAISMGAIGRSNCFHRNGHNAACEASNTTNMIIFAAIQILLSQLPNFHKIWWLSIVAAVMSLAYSSIGLGLSIAKIAGGVHAKTALTGVTVGVDVSASEKIWRTFQSLGDIAFAYSYSNVLIEIQDTLRSSPAENKVMKKASLIGVSTTTTFYMLCGVLGYAAFGSSAPGNFLTGFGFYEPFWLVDIGNVCIIVHLVGAYQVFCQPIYQFVEGWARSRWPDSAFLHAERVLRLPAVLGGGEFPVSPLRLVWRTAYVVLTAVVAMLFPFFNDFLGLIGAVSFWPLTVYFPVEMYMAQAKVRRFSPTWTWMNVLSVACLVVSVLAAAGSVQGLIKDVAGYKPFKVS

SEQ ID NO: 136 - DNA, 2214 bp, Triticum urartu
  EMS56484
atggaggtggtgacggccttgaccaatgttgaggttcctgcgactgggactgtggctgaggctaccgacaggtctgatgctgagagggcgtccaagtgggcgcggtgctggcggatccttggctggacgcttggtgagggcatcgtcggcgaggactttggatggagttggggaggtggagctggtggttgcttctatttcccttacttcacatgtggtcaaggctccggagatgatgactgcgtccgaggtggggcttgggggagggggttcggggcaggtgcgtctcctatgacgacggcgttccactctgctgcgaggggtggggcgggaggagggctcgggcaggtggcccccgccatcctctctcccgacatgcccgtggccctgggcctcggtgtggggcacttgtccgaggggcatgggagcccgcagccgcctgctccggtaaccttggttgaccctttgcgggattcagcgcgaggctttactagggaggaggtcgttgcttttggcgggattcctgacccggtctcggcggggagatggatgagtgctcgcattcaggagcttccggaggttgatgacatgcagcagaggtgcgctatgagggaggccaagcttcatgatgctgagatctctactggttatttttcgagccacggcagtgatccgttcgtggtcgctactcactccgatggaggccagagagcatttggttactggatctatccgctgggagacgctagccagctagaagcaatggggatggagaagggcaaggctgaccctgccaccttcagcatcgctgaggccggctttggagaccggacggacatcgacgacgacggacgcgagaggcgtaccggtacgctggtgacggcgagcgcccacatcatcacggcggtcatcgggtccggggtgctgtcgctggcgtgggccatcgcgcagctcgggtgggtcatcggccccgccgtgctcgtcgccttctccgtcatcacctggttctgctccagcctactggccgactgctaccgctcaccggaccccgtccacggcaagcgcaactacacctacggccaggccgtcagggccaacctaggagttagcaaatacaggctctgctctctggcccaatacgtcaacttggttggcgtcaccattggctacaccatcaccacggccatcagcatgggggcgatcggacggtcgaattgcttccaccgcaatggccacaatgcggcctgcgaggcatccaacaccaccaacatgattatatttgctgccatccaaatcttgctctcgcaactccccaacttccacaagatctggtggctctccattgttgccgccgtcatgtccctcgcctactcctccattggtctcggcctctccatagcaaaaatcgcaggtggggtgcatgccaagacagcgctaacaggggtgaccgttggggtggatgtatccgcgagtgagaaaatttggaggacgttccagtctcttggggacatcgcctttgcatactcctactccaatgtgctcatcgaaatccaggacacgctgcggtcgagcccggcagagaacaaggtgatgaagaaggcgtccttgatcggtgtttccacgaccaccacgttctacatgctgtgcggggtgctgggctacgcggcgttcggcagcagcgccccgggtaacttcctcacgggcttcggcttctacgagcccttctggctcgtcgacatcggcaacgtttgcatcatcgtgcacctcgttggcgcctaccaggtcttctgccagcccatctaccaattcgtcgagggctgggcgcgctcccggtggcccgacagcgccttcctccatgccgagcgcgtgttgcgccttccggccgttctcggaggcggagagttcccggttagcccgttacgcctggtctggcgaacggcgtacgtggttctcacggcggtggtggccatgctgttccccttcttcaacgacttccttggcctcattggcgccgtctcgttttggccgctcaccgtctacttccccgttgagatgtacatggcacaagccaaagtgcgccggttctcgccgacgtggacgtggatgaacgtgcttagcgtcgcgtgccttgtcgtctctgtcctcgccgcagctggttctgtgcaggggctcatcaaggacgtcgcagggtacaagccattcaaggtctcctaa

SEQ ID NO: 137 - protein, 454 aa, Triticum urartu
  EMS68703.1 TRIUR3_33957
MGVLGLIQLVGRRRGEYPLVRDTVTPQGGGESGGGGGGAMDVDGHLPRTHGDVDDDGRERRTGTVWTAAAHIITAVIGSGVLSLAWAMAQLGWVAGPLTLVLFAIITFYTCGLLADRYRVGDPVTGKRNYTYTEAVQAYLGTCSPQARPFLLIKMQPEMMCMCSGGWHVWFCGFCQYVNMFGTGIGYTITASTSAAALKKSNCFHWHGHKADCSQYLSAYIIAFGVVQVIFCQVPNFHKLSWLSIVAAIMSFSYATIAVGLSLAQTISGPRGRTSLTGTEVGVDVDASQKVWMTFQALGNVAFAYSYSIILIEIQDTLRSPPGENKTMRKATLMGISTTTAFYMLCGCLGYSAFGNDANGNILTGFGFYEPYWLVDFANVCIVLHLVGGFQVFCQPLFAAMYIRQRQIPRFGTKWVALQSLSFVCFLVTVAACAASIQGVRDSLKTYTPFTTKS

SEQ ID NO: 138 - DNA, 1365 bp, Triticum urartu
  EMS68703.1 TRIUR3_33957
atgggggtcctcggcctgatccaactggtcgggagacgacgtggtgagtaccccctagtccgggacaccgtcaccccccagggaggcggcgagagcggcggcggcggaggcggggccatggacgtcgacggccaccttccccgcacccacggcgacgtcgacgacgacggcagggagaggagaacagggacggtatggacggcggcggcgcacatcataacggcggtgatcgggtccggcgtgctgtcgctggcctgggccatggcgcagctgggctgggtggccgggccgctcaccctggtgctcttcgccatcatcaccttctacacctgcggcctcctcgccgaccgctaccgcgtcggcgaccccgtcacgggcaagcgcaactacacctacaccgaggccgtccaggcctacctaggtacgtgctcgcctcaagctcgcccgtttttactcatcaaaatgcaacctgagatgatgtgcatgtgttcaggcgggtggcacgtctggttctgcggcttctgccagtacgtcaacatgttcggcaccggcatcggctacaccatcaccgcctccaccagcgccgcggccttgaagaagtccaactgcttccactggcacgggcacaaggcggactgcagccagtacctgagcgcctacatcatcgccttcggggtggtgcaggtcatcttctgccaggtgcccaacttccacaagctctcgtggctctccatcgtcgccgccatcatgtccttctcctacgccaccatcgccgtcggcctctcgctggcgcagaccatctcggggcccagggggaggacgtcgctgaccggcacggaggtcggggtggacgtcgacgcctcgcagaaggtctggatgacgttccaggccctcggcaacgtcgccttcgcctactcctactccataatcctcatcgagatccaggacacgctgcggtcacctccgggcgagaacaagacgatgcggaaggcgacgctgatgggcatctcgacgacgacggccttctacatgctgtgcggctgcctgggctactcggccttcggcaacgacgccaacggcaacatcctgacggggttcggcttctacgagccctactggctggtggacttcgccaacgtctgcatcgtgctccacctggtgggcggcttccaggtcttctgccagccgctgttcgcggcgatgtacatccggcagcggcagatcccgcggttcggcaccaagtgggtggcgctgcagagcctcagcttcgtctgcttcctcgtcaccgtcgccgcctgcgccgcctccatccagggcgtccgcgactcgctcaagacctacacgcccttcaccaccaagtcgtga

SEQ ID NO: 139 - protein, 486 aa, Brassica rapa
  VDC65345.1 unnamed protein product
MSPSPPLTMKSLDTLHNPSAVESGNAAVKNVDDDGREKRTGTFLTASAHIITAVIGSGVLSLAWALAQLGWVAGTMILVIFAIITYYTSTLLADCYRAPDPITGTRNYTYMGVVRAYLGGKKVQLCGLAQYGNLVGVSIGYTITASISLVAIGRANCFHDKGHGAKCTASNYPYMVAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNLSPGDFLTDFGFYEPFWLVIFANVCIAVHLVGAYQVYVQPFFQFVESKCNKKWPESNFINKEYSLKIPLLGKFRVNFFRLVWRTNYVILTTFIAMIFPFFNSILGLLGALAFWPLTVYFPVAMHIAQTKVKKYSGRWLALNLLVLVCLIVSALAAVGSIVGLINNVKKYKPFESID

SEQ ID NO: 140 - DNA, 1461 bp, Brassica rapa
  VDC65345.1 unnamed protein product
atgtctccttctccccctcttacaatgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacgtctactttgctcgccgattgctacagagcgccggaccccatcaccggaacacgcaactacacgtacatgggcgtcgttcgagcttaccttggtggtaaaaaggtgcagctatgtggactagcacagtacggaaacctcgttggggtctctattggttacaccatcactgcctccataagcttagtagcgattgggagagcaaattgttttcatgacaagggacatggtgcgaaatgtaccgcatcgaattatccatacatggtggcatttggcggcctccagattcttctaagtcagattcctaattttcacaagctatctttcctctcaatcattgccgcggttatgtccttctcttatgcatctattggtatcggtctggccatcgccaaagtagcaagtgggaaggttggtaagacaacactgacaggtacggtgataggagtggacgtatctgcgtctgataaagtgtggaaagcgtttcaagcggttggggatattgcgttttcgtacgcttacaccactattctcattgagatacaggacacattgagatcaagcccaccagagaacaaagtgatgaagaaagcaagtcttattggagtctcaaccacaactgttttctacctcttatgtggttgcattggatatgctgcattcggaaacttatcccctggtgacttccttaccgactttgggttttacgaacctttctggctcgtcattttcgccaatgtttgcattgctgtccatttagtaggtgcctatcaggtatatgttcagccttttttccagtttgttgagagcaaatgtaacaaaaagtggcctgaaagcaatttcatcaacaaagaatactcgttgaagataccattgctcggaaaatttcgtgtcaacttcttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgctggtttgcttgattgtctccgccctagctgctgtgggatccattgttggcctaatcaataatgtcaagaaatacaagcctttcgagagtatagactaa

SEQ ID NO: 141 - protein, 486 aa, Brassica rapa; identical to SEQ ID NO: 139
  RID57273.1 hypothetical protein BRARA_F00660

SEQ ID NO: 142 - DNA, 1437 bp, Brassica rapa
  RID57273.1 hypothetical protein BRARA_F00660
atgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacgtctactttgctcgccgattgctacagagcgccggaccccatcaccggaacacgcaactacacgtacatgggcgtcgttcgagcttaccttggtggtaaaaaggtgcagctatgtggactagcacagtacggaaacctcgttggggtctctattggttacaccatcactgcctccataagcttagtagcgattgggagagcaaattgttttcatgacaagggacatggtgcgaaatgtaccgcatcgaattatccatacatggtggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacactgacaggtacggtgataggagtggacgtatctgcgtctgataaagtgtggaaagcgtttcaagcggttggggatattgcgttttcgtacgcttacaccactattctcattgagatacaggacacattgagatcaagcccaccagagaacaaagtgatgaagaaagcaagtcttattggagtctcaaccacaactgttttctacctcttatgtggttgcattgggtatgctgcattcggaaacttatcccctggtgacttccttaccgactttgggttttacgaacctttctggctcgtcattttcgccaatgtttgcattgctgtccatttagtaggtgcctatcaggtatatgttcagccttttttccagtttgttgagagcaaatgtaacaaaaagtggcctgaaagcaatttcatcaacaaagaatactcgttgaagataccattgctcggaaaatttcgtgtcaacttcttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgctggtttgcttgattgtctccgccctagctgctgtgggatccattgttggcctaatcaataatgtcaagaaatacaagcctttcgagagtatagactaa

SEQ ID NO: 143 - protein, 486 aa, Brassica rapa
  XP_009148321.1 AAP8
MSPSPPLTMKSLDTLHNPSAVESGNAAVKNVDDDGREKRTGTFLTASAHIITAVIGSGVLSLAWALAQLGWVAGTMILVIFAIITYYTSTLLADCYRAPDPITGTRNYTYMGVVRAYLGGKKVQLCGRAQYGNLVGVSIGYTITASISLVAIGRANCFHDKGHGAKCTASNYPYMVAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNLSPGDFLTDFGFYEPFWLVIFANVCIAVHLVGAYQVYVQPFFQFVESKCNKKWPESNFINKEYSLKIPLLGKFRVNFFRLVWRTNYVILTTFIAMIFPFFNSILGLLGALAFWPLTVYFPVAMHIAQTKVKKYSGRWLALNLLVLVCLIVSALAAVGSIVGLINNVKKYKPFESID

SEQ ID NO: 144 - DNA, 1461 bp, Brassica rapa
  XP_009148321 AAP8
atgtctccttctccccctcttacaatgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcatcacttactacacgtctactttgctcgccgattgctacagagcgccggaccccatcaccggaacacgcaactacacgtacatgggcgtcgttcgagcttaccttggtggtaaaaaggtgcagctatgtggacgagcacagtacggaaacctcgttggggtctctattggttacaccatcactgcctccataagcttagtagcgattgggagagcaaattgttttcatgacaagggacatggtgcgaaatgtaccgcatcgaattatccatacatggtggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacactgacaggtacggtgataggagtggacgtatctgcgtctgataaagtgtggaaagcgtttcaagcggttggggatattgcgttttcgtacgcttacaccactattctcattgagatacaggacacattgagatcaagcccaccagagaacaaagtgatgaagaaagcaagtcttattggagtctcaaccacaactgttttctacctcttatgtggttgcattgggtatgctgcattcggaaacttatcccctggtgacttccttaccgactttgggttttacgaacctttctggctcgtcattttcgccaatgtttgcattgctgtccatttagtaggtgcctatcaggtatatgttcagccttttttccagtttgttgagagcaaatgtaacaaaaagtggcctgaaagcaatttcatcaacaaagaatactcgttgaagataccattgctcggaaaatttcgtgtcaacttcttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgaacctcctcgtgctggtttgcttgattgtctccgccctagctgctgtgggatccattgttggcctaatcaataatgtcaagaaatacaagcctttcgagagtatagactaa

SEQ ID NO: 145 - protein, 488 aa, Brassica rapa
  RID48756.1 hypothetical protein BRARA_I05242
MLLSLSSLPRFFSSKMKSYATEYNPSAVETAGNNFDDDGREKRTGTLMTATAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHDKGHDAKCSVSNYPLMAAFGITQIVLSQIHNFHKLSFLSIIATVMSFSYASIGIGLALAALASGKVGKTDLTGTVVGVDVTASDKIWRSFQAAGDIAFSYAFSVVLVEIQDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFIAKDYSINIPLLGKCRINFFRLVWRSTYVILTTVVAMIFPFFNAILGLIGALTFWPLTVYFPVEMHISQRKVKKYTMRWIGLKLLVLVCLVVSLLAAVGSIVGLISSVKAYKPFHNLD

SEQ ID NO: 146 - DNA, 1467 bp, Brassica rapa
  RID48756.1 hypothetical protein BRARA_I05242
atgcttttatcactttcttctcttcctcggtttttctcgtctaaaatgaaaagctacgccactgagtataatccctcggccgtggaaaccgccgggaataacttcgacgatgatggtcgggagaagagaacggggacgttgatgacggcgaccgcgcacataatcacggcggtgataggttctggagtcttgtcgttggcttgggctatagcacaacttggttgggtggcaggaacggtgattttggtaacttttgccgttataaattacttcacatctacaatgcttgcggactgctatcgatctccggacacaggaatacgtaattataattacatggacgttgtcagagcttaccttggtggttggaaagtgaagctgtgtggactggcacagtacgggagtctagtagggatcactattggctacaccatcactgcctccataagcttagtagcgatcgggaaagcaaattgttttcatgacaagggacatgatgcaaaatgttccgtatcaaattatccactcatggcggcgtttggtatcacccagattgttcttagtcagattcataattttcacaagctctcttttctctccattatcgctaccgttatgtccttctcttatgcatccatcggaattggcttagccttggctgctctggcaagtgggaaggttggtaagacggatctgacgggcacggtggttggagtagacgtaactgcgtctgacaaaatatggaggtcgtttcaagcagctggagacattgccttttcgtacgcattttccgttgttctcgttgagattcaggatacactgagatcaagcccaccagagaacaaagtcatgaaaaaagcaagccttgctggagtttcaactacaactggtttctacatcttgtgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaacttcatcgccaaagattattcgataaacataccattgctagggaaatgtcgcatcaacttcttcagattggtctggaggtcaacctatgtgattttgacaacagttgtagcgatgatattccccttctttaacgcgatcttgggccttattggagcactcacattctggccgctaacagtttacttcccagtggagatgcacatctcgcagagaaaggttaagaagtatactatgagatggatagggttgaaactccttgtattggtttgtttggttgtttcgctcctagctgcagtaggatccattgtcggcttgataagtagtgtaaaggcatacaagcctttccacaatttagattag

SEQ ID NO: 147 - protein, 488 aa, Brassica rapa
  XP_009118279.1 PREDICTED amino acid permease 8-like
MLLSLSSLPRFFSSKMKSYATEYNPSAVETAGNNFDDDGREKRTGTLMTATAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHDKGHDAKCSVSNYPLMAAFGITQIVLSQIHNFHKLSFLSIIATVMSFSYASIGIGLALAALASGKVGKTDLTGTVVGVDVTASDKIWRSFQAAGDIAFSYAFSVVLVEIQDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFIAKDYSINIPLLGKCRINFFRLVWRSTYVILTTVVAMIFPFFNAILGLIGALTFWPLTVYFPVEMHISQKKIKKYTMRWIGLKLLVLVCLVVSLLAAVGSIVGLISSVKAYKPFHNLD

SEQ ID NO: 148 - DNA, 1467 bp, Brassica rapa
  XP_009118279.1 PREDICTED amino acid permease 8-like
atgcttttatcactttcttctcttcctcggtttttctcgtctaaaatgaaaagctacgccactgagtataatccctcggccgtggaaaccgccgggaataacttcgacgatgatggtcgggagaagagaacggggacgttgatgacggcgaccgcgcacataatcacggcggtgataggttctggagtcttgtcgttggcttgggctatagcacaacttggttgggtggcaggaacggtgattttggtaacttttgccgttataaattacttcacatctacaatgcttgcggactgctatcgatctccggacacaggaatacgtaattataattacatggacgttgtcagagcttaccttggtggttggaaagtgaagctgtgtggactggcacagtacgggagtctagtagggatcactattggctacaccatcactgcctccataagcttagtagcgatcgggaaagcaaattgttttcatgacaagggacatgatgcaaaatgttccgtatcaaattatccactcatggcggcgtttggtatcacccagattgttcttagtcagattcataattttcacaagctctcttttctctccattatcgctaccgttatgtccttctcttatgcatccatcggaattggcttagccttggctgctctggcaagtgggaaggttggtaagacggatctgacgggcacggtggttggagtagacgtaactgcgtctgacaaaatatggaggtcgtttcaagcagctggagacattgccttttcgtacgcattttccgttgttctcgttgagattcaggatacactgagatcaagcccaccagagaacaaagtcatgaaaaaagcaagccttgctggagtttcaactacaactggtttctacatcttgtgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaacttcatcgccaaagattattcgataaacataccattgctagggaaatgtcgcatcaacttcttcagattggtctggaggtcaacctatgtgattttgacaacagttgtagcgatgatattccccttctttaacgcgatcttgggccttattggagcactcacattctggccgctaacagtttacttcccagtggagatgcacatctcgcagaaaaagattaagaagtatactatgagatggatagggttgaaactccttgtattggtttgtttggttgtttcgctcctagctgcagtaggatccattgtcggcttgataagtagtgtaaaggcatacaagcctttccacaatttagattag

SEQ ID NO: 149 - protein, 488 aa, Brassica rapa
  RID48754.1 hypothetical protein BRARA_I05240
MLLHISFLSSSVSPLKMKSFDTSSVVESGAGAGNNVDDDCREKRTGTLITASAHIITTVIGSGVLSLAWAIAQLGWVVGTVILVAFAVIVNYTSRMLADSYRSPEGTRNYTYMDVVRVYLGGRKVQLCGLAQFGSLVGVTIGYTITASISLVAIGKANCFHDKGHGAKCSVSNYPLMAAFGIVQIFLSQIPNFHKLSFLSIIATVMSFSYASIGFGLALAALASGKVGKTGLTGTVVGVDVTASDKLWKSFQAAGNIAFSYAYSVVLVEIQDTLRSSPPENKVMKKASLAAVSTTTAFYILCGCIGYATFGNQAPGDFLTDFGFYEPYWLIDFANACIAVHLIGAYQVFAQPIFQFVEKKCNQAWPESNFITKEHSMNIPLLGKCRINFFRLVWRTTYVIFSTVVAMIFPFFNAILGLIGAVAFWPLTVYFPVEMHISQKKVKKYSVRWIVLKLLVLVCLIVSLLAAIGSIVGLISSVKAYKPFHNLD

SEQ ID NO: 150 - DNA, 1467 bp, Brassica rapa
  RID48754.1 hypothetical protein BRARA_I05240
atgcttttgcatatctcttttctctcttcttcagtttctcctctcaaaatgaaaagcttcgacacgagctcagtggttgaatccggtgctggcgccgggaataacgtcgacgatgattgtcgggagaagagaacggggaccttgataacggcgagtgcccacataatcacgacagtgataggttctggagtcttgtcgttggcttgggctatagcacaacttggttgggtggtaggaacagtgattttggtagcctttgccgtcatagttaattacacatccagaatgctcgccgacagttatcgatccccggagggaacacgcaactatacttacatggacgtcgtccgagtctaccttggtggtaggaaagtgcagctgtgtggactagcacagttcgggagtctcgtaggggttactattggttacaccatcactgcctccataagcttagtggcgattgggaaagcaaattgttttcatgacaagggacatggtgcgaaatgttccgtatcaaattatccactcatggcggcgtttggaatcgtccagatttttcttagtcagattcctaattttcacaagctctcttttctctccattatcgccaccgttatgtccttctcttatgcatctatcggttttggcttagccttggccgctctggcaagtgggaaggttggtaagacgggactgacaggcacggtggttggagtggatgtaactgcgtctgacaaattatggaagtcatttcaagcggctggaaacattgccttttcatacgcttattccgttgttctcgttgagattcaggacacactgagatcaagcccaccagagaacaaagtcatgaaaaaagcaagccttgctgcagtctcaactacaactgctttctacatcttatgtggctgcatcggatatgctacatttggaaaccaagcccccggagacttccttactgactttggtttttatgaaccttactggctcatcgattttgctaatgcttgcatcgctgtccaccttatcggagcttatcaggtgtttgcacaaccaatattccagtttgttgagaagaaatgcaatcaggcgtggccagaaagcaacttcatcaccaaagaacattcgatgaacataccgttgcttggaaaatgtcgcattaacttcttcagactggtgtggaggacaacctatgtgattttctcaacagttgtagcaatgatatttcccttcttcaacgctatcttgggccttattggggcagtcgcattctggccgctaacagtttacttcccggtggagatgcacatctcgcagaaaaaggttaagaagtattctgtgagatggatagtattgaaactccttgttttggtttgtttaattgtttcgctcctagctgccataggatccatcgttggcttgataagtagtgtcaaggcatacaagcctttccacaatttagattag

SEQ ID NO: 151 - protein, 472 aa, Brassica rapa
  XP_009118276.1 AAP8-like isoform X2
MKSFDTSSVVESGAGAGNNVDDDCREKRTGTLITASAHIITTVIGSGVLSLAWAIAQLGWVVGTVILVAFAVIVNYTSRMLADSYRSPEGTRNYTYMDVVRVYLGGRKVQLCGLAQFGSLVGVTIGYTITASISLVAIGKANCFHDKGHGAKCSVSNYPLMAAFGIVQIFLSQIPNFHKLSFLSIIATVMSFSYASIGFGLALAALASGKVGKTGLTGTVVGVDVTASDKLWKSFQAAGNIAFSYAYSVVLVEIQDTLRSSPPENKVMKKASLAAVSTTTAFYILCGCIGYATFGNQAPGDFLTDFGFYEPYWLIDFANACIAVHLIGAYQVFAQPIFQFVEKKCNQAWPESNFITKEHSMNIPLLGKCRINFFRLVWRTTYVIFSTVVAMIFPFFNAILGLIGAVAFWPLTVYFPVEMHISQKKVKKYSVRWIVLKLLVLVCLIVSLLAAIGSIVGLISSVKAYKPFHNLD

SEQ ID NO: 152 - DNA, 1419 bp, Brassica rapa
  XP_009118276.1 AAP8-like isoform X2
atgaaaagcttcgacacgagctcagtggttgaatccggtgctggcgccgggaataacgtcgacgatgattgtcgggagaagagaacggggaccttgataacggcgagtgcccacataatcacgacagtgataggttctggagtcttgtcgttggcttgggctatagcacaacttggttgggtggtaggaacagtgattttggtagcctttgccgtcatagttaattacacatccagaatgctcgccgacagttatcgatccccggagggaacacgcaactatacttacatggacgtcgtccgagtctaccttggtggtaggaaagtgcagctgtgtggactagcacagttcgggagtctcgtaggggttactattggttacaccatcactgcctccataagcttagtggcgattgggaaagcaaattgttttcatgacaagggacatggtgcgaaatgttccgtatcaaattatccactcatggcggcgtttggaatcgtccagatttttcttagtcagattcctaattttcacaagctctcttttctctccattatcgccaccgttatgtccttctcttatgcatctatcggttttggcttagccttggccgctctggcaagtgggaaggttggtaagacgggactgacaggcacggtggttggagtggatgtaactgcgtctgacaaattatggaagtcatttcaagcggctggaaacattgccttttcatacgcttattccgttgttctcgttgagattcaggacacactgagatcaagcccaccagagaacaaagtcatgaaaaaagcaagccttgctgcagtctcaactacaactgctttctacatcttatgtggctgcatcggatatgctacatttggaaaccaagcccccggagacttccttactgactttggtttttatgaaccttactggctcatcgattttgctaatgcttgcatcgctgtccaccttatcggagcttatcaggtgtttgcacaaccaatattccagtttgttgagaagaaatgcaatcaggcgtggccagaaagcaacttcatcaccaaagaacattcgatgaacataccgttgcttggaaaatgtcgcattaacttcttcagactggtgtggaggacaacctatgtgattttctcaacagttgtagcaatgatatttcccttcttcaacgctatcttgggccttattggggcagtcgcattctggccgctaacagtttacttcccggtggagatgcacatctcgcagaaaaaggttaagaagtattctgtgagatggatagtattgaaactccttgttttggtttgtttaattgtttcgctcctagctgccataggatccatcgttggcttgataagtagtgtcaaggcatacaagcctttccacaatttagattag

SEQ ID NO: 153 - protein, 486 aa, Brassica rapa; identical to SEQ ID NO: 139
  RID57272.1 hypothetical protein BRARA_F00659

SEQ ID NO: 154 - DNA, 1413 bp, Brassica rapa
  RID57272.1 hypothetical protein BRARA_F00659
atgaaaagctttgacgcggtgcataatccctctgcggtggaatccgctgacgccaacgtcgacgatgatggtcgggagaagagaacggggacgttgatgacggcgagtgcgcacataatcacggctgtgataggttccggagtgttgtcgttggcttgggctatagcacaacttggttgggtggcaggaacattgattcttgtaacttttgccgtcgtcaattactacacatccactatgctcgccgattgttatagatcggacgcaggagctcgcaactatacgtacatggacgtcgttcgatcttaccttggtggtaggaaagtgcagttatgtggactggcacaatacgggtgtctcgtaggggtcactattggttacaccatcactgcgtctataagtttagtagcgatttggaaagcaacttgttttcataaaaaaggacatggtgcaaaatgctccatcccaaattatccattcatggtggccttcggggtcgtggagattcttcttagtcagcttcctaattttcacaagctctcttttctctccattatcgccgccattatgtcattctcttatgcgtctatcggaattggtttagccatttccgttgtggcaagtggaaaggttggtaagacgagtgtgacgggcacggtggttggagtggacgtgaccgcatctgacaaaatatggaaggcgtttcaagcaactggagacattgcattttcatactctttttccactattctcgttgagattcaggatacattgagatcaaacccaccagaaaacaaagtcatgaaaaaagcaacacttgccggagtctcaactacaactgttttctacatcttatgtggctgcatgggatatgctgcatttggaaaccgagcccccggagacttccttactgactttggtttttatgaaccttactggctcatcaattttgccaatgcttgcatcgtcctccacctaattgcagcctatcaggtgtttgcacaaccaattttccaacttgttgagaacaaatgcaacaaagcatggccagaaaacaatttcatccacaaagaacattcgataaacatactattcctcggaaaatggcgcatcaacttcttcagactggtgtggaggacagcatatgtgattttgacaacatttgttgcagtgatattccccttcttcaactcgatcttgggccttatcggagcaacagcattctggccgctaacagtttacttcccagtggagatgcacatctcgcagagaaaggttaagaagtattctatgaaatggaatgcgttgaaactccttatatcggtttgtttgattgtttcgctcctagctgcaataggatccattgtcggcttgataaatagtgtcaaggcatacaagcctttccatagttaa

SEQ ID NO: 155 - protein, 485 aa, Brassica oleracea
  VDD42023.1 unnamed protein product
MSPSPPPTMKSLDTLHNPSAVESGNAAVKNVDDDGREKRTGTFLTASAHIITAVIGSGVLSLAWALAQLGWVAGTMILVIFAIITYYTSTLLADCYRAPDPITGTRNYTYMGVVRAYLGGKKVQLCGLAQYGNLVGVSIGYTITASISLVAIGKANCFHGKGHGAKCTASNYPYMVAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNIAPGDFLTDFGFYEPFWLVIFANVCIAVHLVGAYQVYVQPFFQFVESKCNKKWPESNFINKEYSLKIPLLGKFRVNHFRLVWRTNYVILTTFIAMIFPFFNSILGLLGALAFWPLTVYFPVAMHISQTKVKKYSGRWLALNLLVLVCLIVSALAAVGSIVGLINNVKKYKPFESI

SEQ ID NO: 156 - DNA, 1461 bp, Brassica oleracea
  VDD42023.1 unnamed protein product
atgtctccctctccccctcctacaatgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcattacttactacacctctactttgctcgccgattgctacagagcgccggaccccatcaccggaacacgcaactacacgtacatgggcgtcgttcgagcttaccttggtggtaaaaaggtgcagctatgtggactagcacagtacggcaacctcgttggggtctctattggttacaccatcactgcctccataagcttagtagcgattgggaaagcaaattgttttcatggtaagggacatggtgcgaaatgtaccgcatcgaattatccatacatggtggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacgctgacaggtacggtgataggagtggacgtatctgcgtctgataaagtatggaaagcgtttcaagcggttggggatattgcgttttcgtacgcttacaccactattctcattgagatccaggacacattgagatcaagcccaccagagaacaaagtgatgaagaaagcaagtcttattggagtctcaaccacaactgttttctacctcttatgtggttgcattgggtatgctgcattcggaaacatagcccctggtgacttccttaccgactttgggttttacgaacctttctggctcgtcattttcgccaatgtttgcattgctgtccatttagtaggtgcctatcaggtatatgttcagccctttttccaatttgttgagagcaaatgcaacaaaaagtggcctgaaagcaatttcatcaacaaagagtactcgttgaagataccattgctcggaaaatttcgtgtcaaccacttcaggctggtgtggaggacaaactatgtgattttgacaacatttattgcaatgatattccccttcttcaactccatcttgggtttgcttggggcacttgcgttctggccgttaacagtttattttcctgtggcaatgcacatttctcagacaaaggttaagaagtattcgggtagatggttggcgctgaacctcctcgtgttggtttgcttgattgtctccgccttagctgcagtgggatccattgttggtctaatcaataatgtcaagaaatacaagcctttcgagagtatagactaa

SEQ ID NO: 157 - protein, 486 aa, Brassica oleracea var. oleracea
  XP_013586575.1 PREDICTED amino acid permease 8
MSPSPPPTMKSLDTLHNPSAVESGNAAVKNVDDDGREKRTGTFLTASAHIITAVIGSGVLSLAWALAQLGWVAGTMILVIFAIITYYTSTLLADCYRAPDPITGTRNYTYMGVVRAYLGGKKVQLCGLAQYGNLVGVSIGYTITASISLVAIGKANCFHGKGHGAKCTASNYPYMGAFGGLQILLSQIPNFHKLSFLSIIAAVMSFSYASIGIGLAIAKVASGKVGKTTLTGTVIGVDVSASDKVWKAFQAVGDIAFSYAYTTILIEIQDTLRSSPPENKVMKKASLIGVSTTTVFYLLCGCIGYAAFGNIAPGDFLTDFGFYEPFWLVIFANVCIAVHLVGAYQVYVQPFFQFVESKCNKKWPESNFINKEYSLKIPLLGKFRVNHFRLVWRTNYVILTTFIAMIFPFFNSILGLLGALAFWPLTVYFPVAMHIAQTKVKKYSGRWLALHLLVLVCLIVSALAAVGSIVGLINNVKKYKPFESID

SEQ ID NO: 158 - DNA, 1461 bp, Brassica oleracea var. oleracea
  XP_013586575.1 PREDICTED amino acid permease 8
atgtctccctctccccctcctacaatgaaatccttggacacactccacaatccctcggcggttgagtccggtaacgccgctgtgaagaacgtcgacgatgatggtcgagagaagagaacggggacgtttctgacggcgagtgcgcacattatcacggcggtgataggctcaggagtgttgtctttggcttgggcattagcacagcttggttgggtggctggaaccatgattttggtgattttcgccatcattacttactacacctctactttgctcgccgattgctacagagcgccggaccccatcaccggaacacgcaactacacgtacatgggcgtcgttcgagcttaccttggtggtaaaaaggtgcagctatgtggactagcacagtacggcaacctcgttggggtctctattggttacaccatcactgcctccataagcttagtagcgattgggaaagcaaattgttttcatggtaagggacatggtgcgaaatgtaccgcatcgaattatccatacatgggggcatttggcggcctccagattcttctaagtcagattcctaattttcacaagctatctttcctctcaatcattgccgcggttatgtccttctcttatgcatctattggtatcggtctggccatcgccaaagtggcaagtgggaaggttggtaagacaacgctgacaggtacggtgataggagtggacgtatctgcgtctgataaagtatggaaagcgtttcaagcggttggggatattgcgttttcgtacgcttacaccactattctcattgagatccaggacacattgagatcaagcccaccagagaacaaagtgatgaagaaagcaagtcttattggagtctcaaccacaactgttttctacctcttatgtggttgcattgggtatgctgcattcggaaacatagcccctggtgacttccttaccgactttgggttttacgaacctttctggctcgtcattttcgccaatgtttgcattgctgtccatttagtaggtgcctatcaggtatatgttcagccctttttccaatttgttgagagcaaatgcaacaaaaagtggcctgaaagcaatttcatcaacaaagagtactcgttgaagataccattgctcggaaaatttcgtgtcaaccacttcaggctggtgtggaggacaaactatgtgattttgacaacatttattgcaatgatattccccttcttcaactccatcttgggtttgcttggggcacttgcgttctggccgttaacagtttattttcctgtggcaatgcacattgctcagacaaaggttaagaagtattcgggtagatggttggcgctgcacctcctcgtgttggtttgcttgattgtctccgccttagctgcagtgggatccattgttggcctaatcaataatgtcaagaaatacaagcctttcgagagtatagactaa

SEQ ID NO: 159 - protein, 473 aa, Brassica oleracea var. oleracea
  XP_013599620.1 PREDICTED amino acid permease 8-like
MKSFHTEYNPSAVEAAGNNFDDDGREKRTGTVMTASAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHEKGHGAKCSVSNYPLMAAFGIIQIVLSQIHNFHKLSFLSIIATVMSFSYASIGIGLALAALASGKVGKTDLTGTVVGVDVTASDKIWRSFQAAGDIAFSYAFSVVLVEIQDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFITKDYSINIPLLGKCRINFFRLVWRSTYVILTTVAAMIFPFFNAILGLIGALTFWPLTVYFPVEMHISQKKVKKYTMRWIGLKLLVLVCLVVSLLAAVGSIVGLISSVKAYKPFHNLD

SEQ ID NO: 160 - DNA, 1422 bp, Brassica oleracea var. oleracea
  XP_013599620.1 PREDICTED amino acid permease 8-like
atgaaaagcttccacactgagtataatccctcggccgtggaagccgccgggaataacttcgacgacgatggtcgggagaagagaacggggacggtgatgacggcaagtgctcacattatcactgctgtgataggttccggagtcttgtccttggcttgggctatagcacaacttggttgggtggcaggaacagtgattttggtaacttttgccgttataaattacttcacatctacaatgcttgccgactgttatcgatctccggacacaggaatacgtaattataattacatggacgttgtcagagcttaccttggtggttggaaagtgaagctatgtggtctggcacagtacgggagtctagtagggatcactattggttacaccatcactgcctccataagcttagtagcgatagggaaagcaaattgttttcatgaaaagggacatggtgcaaaatgttccgtatcgaattatccactcatggcggcgtttggtatcatccagattgttcttagtcagattcataattttcacaagctctcttttctctccattatcgccaccgttatgtccttctcttatgcatccatcggaattggcttggccttggccgctctggcaagtgggaaggttggtaagacggatctgacgggcacggtggttggagtagacgtaactgcgtctgacaaaatatggaggtcgtttcaagcagctggagacattgccttttcgtacgcattttccgttgttctcgttgagattcaggatacactgagatcaagcccaccagagaacaaagtcatgaaaaaagcaagccttgctggagtttcaactacaactggtttctacatcttatgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaactttatcaccaaagattattcgataaacataccattgctagggaaatgtcgcatcaacttcttcagattggtctggaggtcaacctatgtgattttgacaacagttgcagcaatgatattccccttcttcaacgcgatcttgggccttatcggagcactcacattctggccgctaacagtttacttcccagtggagatgcacatctcgcagaaaaaggttaagaagtatactatgagatggatagggttgaaactccttgtattggtttgtttggttgtttcgctcctagctgcagtaggatccattgtcggcctcataagtagtgtaaaggcatacaagcctttccacaatttagattag

SEQ ID NO: 161 - protein, 470 aa, Brassica oleracea var. oleracea
  XP_013584691.1 PREDICTED amino acid permease 8-like
MKSFDAVHNPSAVESADANVDDDGREKRTGTLMTASAHIITAVIGSGVLSLAWAIAQLGWVAGTLILVTFAIVNYYTSTMLADCYRSDAGARNYTYMDVVRSYLGGRKVQLCGLAQYGCLVGVTIGYTITASISLVAIWKATCFHKKGHGAKCSIPNYPFMAAFGVVEIFLSQLPNFHKLSFLSIIAAVMSFSYASIGIGLAIAVVASGKVGKTGVTGTVVGVDVTASDKIWKAFQATGDIAFSYSFSTILVEIQDTLRSSPPENKVMKKATLAGVSTTTVFYILCGCMGYAAFGNRAPGDFLTDFGFYEPYWLINFANACIVLHLIAAYQVFAQPIFQLVENKCNKAWPENNFINKEHSINIPFLGKWRINFFRLVWRTAYVILTTFVAVIFPFFNSILGLIGATAFWPLTVYFPVEMHISQRKVKKFSMKWNALKLLVLVCLIVSLLAAIGSIVGLINSVKAYKPFHS

SEQ ID NO: 162 - DNA, 1413 bp, Brassica oleracea var. oleracea
  XP_013584691.1 PREDICTED amino acid permease 8-like
atgaaaagctttgacgcggtgcataatccctctgcggtggaatccgctgacgccaacgtcgacgatgatggtcgggagaagagaacggggacgttgatgacggcgagtgcgcacataatcacggcggtgataggttccggagtgttgtcgttggcctgggctatagcacagcttggttgggtggcaggaacactgattcttgtaacttttgccatcgtcaattactacacatccactatgctcgccgactgttatagatcggacgcaggagctcgcaactatacgtacatggacgtcgtccgatcttaccttggtggtaggaaagtgcagttatgtggactggcacaatacgggtgtctcgtaggggtcactattggttacaccatcactgcctctataagtttagtagcgatttggaaagcaacttgttttcataaaaaaggacatggtgcgaaatgttccatcccaaattatccattcatggcggccttcggggtcgtggagatttttcttagtcagcttcctaattttcacaagctctcttttctctccattatcgccgccgttatgtcattctcttatgcgtctatcggaattggtttagccattgccgttgtggcaagtggaaaggttggtaagacgggtgtgacgggcacggtggttggagtggacgtgaccgcatctgacaaaatatggaaggcgtttcaagcaactggagacattgcattttcatactctttttccactattctcgttgagattcaggatacattgagatcaagcccaccagaaaacaaagtcatgaaaaaagcaacactcgccggagtctcaactacaactgttttctacatcttatgtggctgcatgggatatgctgcatttggaaaccgagcccccggagacttccttactgactttggtttttatgaaccttactggctcatcaactttgccaatgcttgcatcgtcctccacctaatcgcagcctatcaggtgtttgcacaaccaattttccaacttgttgagaacaaatgcaacaaagcatggccagaaaacaatttcatcaacaaagaacattcgataaacataccattcctcggaaaatggcgcatcaacttcttcagactggtgtggaggacagcatatgtgattttgacaacatttgttgcagtgatattccccttcttcaactcgatcttgggccttatcggagcaacagcgttctggccgctaacagtttacttcccagtggagatgcacatctcgcagagaaaggttaagaagttttctatgaaatggaatgcgttgaaactccttgtattggtttgtttgattgtttcgctcctagctgcaataggatccatcgtcggcttgataaatagtgtcaaggcatacaagcctttccatagttaa

SEQ ID NO: 163 - protein, 488 aa, Brassica oleracea var. oleracea
  XP_013601938.1 AAP8-like
MLLHISFISSSVSPLKMKSFDTSSVVESGAGAGNNVDDDCREKRTGTLITASAHIITTVIGSGVLSLAWAIAQLGWVVGTVILVAFAVIVNYTSRMLADSYRSPEGTRNYTYMDVVRVYLGGRKVQLCGLAQFGSLVGVTIGYTITASISLVAIGKANCFHDKGHGAKCSVSNYPLMAAFGIVQIFLSQIPNFHKLSFLSIIATVMSFSYASIGFGLALAALASGKVGKTGLTGTVVRVDVTASDKLWKSFQAAGNIAFSYAYSVVLVEIQDTLRSSPPENKVMKKASLAAVSTTTAFYILCGCIGYATFGNQAPGDFLTDFGFYEPYWLIDFANACIAVHLIGAYQVFAQPIFQFVEKKCNQAWPESNFITKEPSMNVPLLGKCRINFFRLVWRTTYVIFSTVVAMIFPFFNAILGLIGAVAFWPLTVYFPVEMHISQKKVKKYSVRWIVLKLLVLVCLIVSLLAAIGSIVGLISSVKAYKPFHNLD

SEQ ID NO: 164 - DNA, 1467 bp, Brassica oleracea var. oleracea
  XP_013601938.1 AAP8-like
atgcttttgcatatctcttttatctcttcttcagtttctcctctcaaaatgaaaagcttcgacacgagctcagtggttgaatccggtgctggcgccgggaataacgtcgacgatgattgtcgggagaagagaacggggacgttgataacggcgagtgcccacataatcacgacagtgataggttctggagtcttgtcgttggcttgggctatagcacaacttggttgggtggtaggaacagtgattttggtagcctttgccgtcatcgttaattacacatccagaatgctcgccgacagttatcgatccccggagggaacacgcaactatacttacatggacgttgtccgagtctaccttggtggtaggaaagtgcagctatgtggactggcacagtttgggagtctcgtaggggttactattggttacaccatcactgcctccataagcttagtggcgattgggaaagcaaattgttttcatgacaagggacatggtgcgaaatgttccgtatcaaattatccactcatggcggcgtttgggatcgtccagatttttcttagtcagattcctaattttcacaagctctcttttctctccattatcgccaccgttatgtccttctcttatgcatctatcggttttggcttagccttggccgctctggcaagtgggaaggttggtaagacgggactgacaggcacggtggttcgagtggacgtaactgcgtctgacaaattatggaagtcatttcaagcggctggaaacattgccttttcatacgcttattccgttgttctcgttgagattcaggacacactgagatcaagcccaccagagaacaaagtcatgaaaaaagcaagccttgctgcagtctcaactacaactgctttctacatcttatgtggctgcatcggatatgctacatttggaaaccaagcccccggagacttccttactgactttggtttttatgaaccttactggctcatcgattttgctaatgcttgcatcgctgtccaccttatcggagcttatcaggtgtttgcacaaccaatattccagtttgttgagaagaaatgcaatcaggcgtggccagaaagcaacttcatcaccaaagaaccttcgatgaacgtaccgttgcttggaaaatgtcgcattaacttcttcagactggtgtggaggacaacctatgtgattttctcaacagttgtagcaatgatattccccttcttcaacgctatcttgggacttattggggcagtcgcattctggccgctaacagtttacttcccggtggagatgcacatctcgcagaaaaaggttaagaagtattcggtgagatggatagtgttgaaactccttgttttggtttgtttaattgtttcactcctagctgccataggatccatcgttggcttgataagtagtgtcaaggcatacaagcctttccacaatttagattag

SEQ ID NO: 165 - protein, 467 aa, Brassica cretica
  RQL92522.1 hypothetical protein DY000_00018764
MKTFHTEYSPSAVETAGNNFDDDGREKRTGTLMTATAHIITAVIGSGVLSLAWAIAQLGWVAGTVILVTFAVINYFTSTMLADCYRSPDTGIRNYNYMDVVRAYLGGWKVKLCGLAQYGSLVGITIGYTITASISLVAIGKANCFHEKGHGAKCSVSNYPLMAAFGIIQIVLSQIHNFHKLSFLSIIATVMSFSYASVGIGLALAALASGKVGKTDLTGTVVGVDVTASDKIWKSFQAAGDIAFSYAFSVDTLRSSPPENKVMKKASLAGVSTTTGFYILCGCIGYAAFGNQAPGDFLTDFGFYEPYWLIDFANACIAVHLIAAYQVFAQPIFQFIEKKCNKAWPESNFITKDYSINIPLLGKCRINFFRLVWRSTYVILTTVVAMIFPFFNAILGLIGALTFWPLTVYFPVEMHISQKKVKKYTMRWIGLKLLVLVCLVVSLLAAIGSIVGLISSVKAYKPFHNLD

SEQ ID NO: 166 - DNA, 1404 bp, Brassica cretica
  RQL92522.1 hypothetical protein DY000_00018764
atgaaaaccttccacactgagtatagtccctcggccgtggaaaccgccgggaataacttcgacgatgatggtcgggagaagagaacggggacgttgatgacggcgaccgcgcacataatcacggcggtgataggttctggagtcttgtcgttggcttgggctatagcacaacttggttgggtggcaggaacggtgattttggtaacttttgccgttataaattacttcacatctacaatgcttgccgactgttatcgatccccggacacaggaatacgtaattataattacatggacgttgtccgagcttaccttggtggttggaaggtaaagttatgtggactggcacagtacgggagtctagtagggattactattggttataccatcactgcctccataagcttagtagcgatcgggaaagcaaattgttttcatgaaaagggacatggtgcaaaatgttccgtatcaaattatccactcatggcggcgtttggtatcatccagattgttcttagtcaaattcataattttcacaagctctcttttctctccattatcgccacggttatgtccttctcttatgcatctgtcggaattggcttagccttggccgctctggcaagtgggaaggttggtaagacggatctgacgggcacggtggttggagtagacgtaactgcgtctgacaaaatatggaagtcattccaagcagctggagacattgccttttcgtatgcattttccgttgatacactgagatcaagcccaccagagaacaaagtcatgaaaaaagcaagccttgctggagtttcaactacaactggtttctacatcttatgtggctgcatcggatatgctgcttttggaaaccaagcccctggagacttcctaactgactttggtttttatgagccttactggctcattgattttgctaatgcttgcattgctgtccacctaatcgcagcctatcaggtgtttgcacaaccaatattccagtttattgagaagaaatgcaacaaagcgtggccagaaagcaactttatcaccaaagattattcgataaacataccattgctagggaaatgtcgcatcaacttcttcagattggtctggaggtcaacctatgtgattttgacaacagttgtagcaatgatattccccttcttcaacgcgatcttgggccttatcggagcactcacattctggccgctaacagtttacttcccagtggagatgcacatctcgcagaaaaaggttaagaagtatactatgagatggatagggttgaaactccttgtattggtttgtttggttgtttcgctcctagctgccataggatccatcgttggcttgataagtagtgtaaaggcatacaagcctttccacaatttagattag

SEQ ID NO: 167 - protein, 8 aa, Artificial Sequence
  AAP polypeptide conserved motif where Xaa is any amino acid but preferably is an A, S or G.
  VARIANT 1
XFWPLTVY

SEQ ID NO: 168 - protein, 435 aa, Artificial Sequence
  Aa_trans motif
RTGTFWTASAHIITAVIGSGVLSLAWAIAQLGWVAGTTVLVAFAIITYYTSTLLADCYRSPDSITGTRNYNYMGVVRSYLGGKKVQLCGVAQYVNLVGVTIGYTITASISLVAIGKSNCYHDKGHKAKCSVSNYPYMAAFGIVQIILSQLPNFHKLSFLSIIAAVMSFSYASIGIGLAIATVASGKIGKTELTGTVIGVDVTASEKVWKLFQAIGDIAFSYAFTTILIEIQDTLRSSPPENKVMKRASLAGVSTTTVFYILCGCIGYAAFGNQAPGDFLTDFGFYEPYWLIDFANACIALHLIGAYQVYAQPFFQFVEENCNKKWPQSNFINKEYSSKVPLLGKCRVNLFRLVWRTCYVVLTTFVAMIFPFFNAILGLLGAFVFWPLTVYFPVAMHIAQAKVKKYSRRWLALNLLVLVCLIVSALAAVGSIIGLI

SEQ ID NO: 169 - DNA, 307 bp, Artificial Sequence
  MUM4 promoter
gacggtggcattaagcatcttgcattgaatgatccgttatatataatctcaggttttttttgggttgaaatgatgatattaaattttaggttgacatgtacttatctttgtaatcaactaattaaatatttgaactgacatgtctacgttatatcataaataaaccaggtgttttaattaaataccacgattaaccttctaaaataaggaaaatcatattttattcgtcaatcactataatttggaaaacgatgcaatatatttatttctttctttatacacatacttaattaattatcaaaatttc

(19) United States
(12) Patent Application Publication
(10) Pub. No.: US 2022/0396804 A1
(43) Pub. Date: Dec. 15, 2022

(54) METHODS OF IMPROVING SEED SIZE AND QUALITY

(71) Applicant: lnstitute of Genetics and Developmental Biology Chinese Academy of Sciences, Beijing (CN)

(72) Inventors: Yunhai LI, Beijing (CN); Shan JIANG, Beijing (CN); Ximing JIN, Beijing (CN)

(21) Appl. No.: 17/640,466

(22) PCT Filed: Sep. 4, 2020
(86) PCT No.: PCT/EP2020/074858

§ 371 Date: Mar. 4, 2022

(30) Foreign Application Priority Data
Sep. 5, 2019 (CN) PCT/CN2019/104566

(51) Int. Cl. C12N 15/82; C12Q 1/6895; C07K 14/415

(52) U.S. Cl. CPC C12N 15/8262; C12N 15/8251; C12Q 1/6895; C07K 14/415; C12Q 2600/13; C12Q 2600/156

(57) ABSTRACT

The invention relates to a method of increasing seed size and/or seed quality in a plant, the method comprising increasing the permease activity of an amino acid permease (AAP). The invention also relates to method of making such plants as well as plants that display an increased seed size and/or seed quality.

### FIELD OF THE INVENTION

The invention relates to a method of increasing seed yield in a plant, the method comprising increasing the permease activity of an amino acid permease (AAP). The invention also relates to a method of making such plants as well as plants that display an increase in seed yield.

### BACKGROUND OF THE INVENTION

Seed size and weight are associated with seed yield, thereby determining seed production in crops. Seed size is also recognized as a critical factor for evolutionary adaption. Seedlings from large seeds have been proposed to possess stronger ability to survive under stress conditions, while plant species with small seeds have been suggested to have a better ability to propagate progeny. A mature seed contains the maternal integuments, the endosperm and the embryo. The complex interactions between the maternal tissues, the endosperm and the embryo regulate seed growth and determine seed size and weight in plants.

The analysis of seed mutants has identified several important regulators of seed size in Arabidopsis. Several of these regulators have been reported to regulate seed size by influencing cell proliferation in maternal tissues, such as KLU/CYTOCHROME P450 78A5 (CYP78A5), ubiquitin-dependent protease DA1, E3 ubiquitin ligases BIG BROTHER (BB) and DA2, transcription factors AUXIN RESPONSE FACTOR 2 (ARF2) and NGAL2, and UBIQUITIN SPECIFIC PROTEASE 15 (UBP15). By contrast, transcription factors TESTA GLABRA 2 (TTG2) and APETALA2 (AP2) may act maternally to regulate seed size by influencing cell expansion. The development of zygotic tissues also affects seed growth. MINISEED3 (MINI3) and HAIKU (IKU) regulate endosperm cellularization, thereby influencing seed size. SHORT HYPOCOTYL UNDER BLUE1 (SHB1) can bind to the promoters of IKU2 and MINI3 and promotes their expression.

Seed size is often controlled by quantitative trait loci (QTLs) (Alonso-Blanco et al., 1999; Song et al., 2007). In Arabidopsis, several quantitative trait loci (QTLs) for seed size have been mapped, but the genes corresponding to these QTLs have not been cloned so far. Seed quality, and in particular, free amino acid and protein content is an important contributor to seed yield. Increasing grain protein levels has significant value when growing grain crops for animal feed or for use in human consumption (such as bread-making or brewing) However, developing high quality seeds is precluded by the inverse relationship between seed quality (in particular protein content) and size.

The present invention addresses the need to enhance seed size and improve seed quality of commercially value crops, such as wheat, rice and maize, for example.

### SUMMARY OF THE INVENTION

Here we report a major QTL gene for seed size and weight on chromosome 1 (SSW1) in Arabidopsis, which encodes an amino acid permease (AAP), specifically AtAAP8. Amino acids are an important source of organic nitrogen in most plant species, and the delivery of nitrogen to sinks is crucial for seed development.

Our findings identify the first gene corresponding to the QTL for seed size, weight and quality (SSW1/AAP8) in Arabidopsis and demonstrate that natural allelic variation in SSW1/AAP8 contributes to the amino acid transport activity of SSW1/AAP8, thereby regulating seed size, weight and quality. In particular, Arabidopsis accessions possess three types of natural allelic variation in the SSW1/AAP8 gene, including SSW1^{Cvi}, SSW1^{Ler} and SSW1^{Col-0} types. The SSW1^{Cvi} allele produces larger and heavier seeds with more free amino acids and storage proteins than the SSW1^{Ler} allele. SSW1^{Cvi} has similar amino acid transport activity to SSW1^{Col-0} and possesses higher amino acid transport activity than SSW1^{Ler}. We have further found that natural variation in the amino acid (A410V) is predominantly responsible for the observed differences in the amino acid transport activity of the SSW1 types. We have also found that loss of function of SSW1/AAP8 causes small and light seeds.

Our results reveal that AAP8 is an important molecular and genetic basis for natural variation in seed size, weight and quality control, and show that this gene is an important target to improve both seed weight and quality in plants.

Accordingly, in a first aspect of the invention, there is provided a method of increasing seed yield in a plant, the method comprising increasing the activity of amino acid permease (AAP). Preferably, an increase in seed yield comprises an increase in seed size and/or seed quality, preferably an increase in seed size and quality.

In one embodiment, the method comprises increasing the expression of AAP8, wherein the amino acid sequence of AAP8 comprises a sequence as defined in SEQ ID NO: 2, 3 or 4 or a functional variant or homologue thereof. Most preferably, the amino acid sequence of AAP8 comprises SEQ ID NO: 4 or a functional variant or homologue thereof.

In one embodiment, the method comprises introducing and expressing a nucleic acid construct, wherein the construct comprises a nucleic sequence encoding an AAP8 polypeptide as defined in SEQ ID NO: 2, 3 or 4 or a functional variant or homologue thereof. Preferably, the nucleic acid sequence is operably linked to a regulatory sequence. More preferably, the regulatory sequence is a constitutive or tissue-specific promoter, such as the MUM4 promoter.

In an alternative embodiment, the method comprises introducing at least one mutation into the plant genome, wherein said mutation increases the activity of an AAP polypeptide. Preferably, the mutation is introduced using targeted genome editing. More preferably, the targeted genome editing is CRISPR.

In one embodiment, the mutation is the insertion of at least one additional copy of a nucleic acid sequence encoding an AAP8 polypeptide or a homolog or functional variant thereof, such that the nucleic acid sequence is operably linked to a regulatory sequence, and wherein the mutation is introduced using targeted genome editing and wherein preferably the nucleic acid sequence encodes an AAP polypeptide as defined in SEQ ID NO: 2, 3 or 4 or a functional variant or homolog thereof.

In an alternative embodiment, the method comprises or results in introducing at least one mutation at position 410 of SEQ ID NO: 1 or at a homologous position in a homologous sequence. Preferably, the mutation is a substitution.

In another aspect of the invention, there is provided a genetically altered plant, part thereof or plant product, wherein the plant is characterised by an increase in seed yield.

Preferably, the genetically altered plant, part thereof or plant product has increased activity of an AAP polypeptide.

In one embodiment, the plant expresses a nucleic acid construct comprising a nucleic acid encoding an AAP8 polypeptide as defined in any of SEQ ID NO: 2, 3 or 4 or a functional variant or homologue thereof.

In an alternative embodiment, the plant has at least one mutation in its genome, wherein the mutation increases the activity of AAP8. Preferably, the mutation is introduced by targeted genome editing, preferably CRISPR.

In one embodiment, the mutation is the insertion of at least one or more additional copy of a nucleic acid encoding an AAP8 polypeptide as defined in SEQ ID NO: 2, 3 or 4 or homolog or functional variant thereof. Alternatively, the mutation is at position 410 of SEQ ID NO: 1 or at a homologous position in a homologous sequence.

In another aspect of the invention, there is provided a method of making a transgenic plant having an increase in seed yield, the method comprising introducing and expressing a nucleic acid construct comprising a nucleic acid sequence encoding an AAP8 polypeptide as defined in SEQ ID NO: 2, 3 or 4 or a functional variant or homolog thereof.

In a further aspect of the invention, there is provided a method of making a genetically altered plant having an increase in seed yield, the method comprising introducing a mutation into the plant genome to increase the activity of an AAP8 polypeptide. Preferably, the mutation is introduced using targeted genome editing, preferably CRISPR.

In one embodiment, the mutation is the insertion of one or more additional copies of a nucleic acid encoding an AAP8 polypeptide as defined in SEQ ID NO: 2, 3 or 4 or a functional variant or homolog thereof, such that the sequence is operably linked to a regulatory sequence. In an alternative embodiment, the method comprises or results in introducing at least one mutation at position 410 of SEQ ID NO: 1 or at a homologous position in a homologous sequence. Preferably, the mutation is a substitution.

In a further aspect of the invention, there is provided a method of screening a population of plants and identifying and/or selecting a plant that has or will have increased activity of a AAP polypeptide, the method comprising detecting in the plant germplasm at least one polymorphism in the nucleic acid encoding an AAP polypeptide or detecting at least one polymorphism in an AAP protein and selecting said plant or progeny thereof.

In one embodiment, the polymorphism is a substitution. Preferably, the substitution is at position 410 of SEQ ID NO: 1, 2, 3 or 4 or position 2635 of SEQ ID NO: 5, 6, 7 or 8 or a homologous substitution in a homologous sequence.

In one embodiment, a “homologous substitution in a homologous sequence” in any of the aspects of the invention described herein, may be selected from one or more of the positions in one of the homologous sequences defined in Table 12.

In a further aspect of the invention there is provided a nucleic acid construct comprising a nucleic acid sequence encoding a AAP8 polypeptide as defined in SEQ ID NO: 2, 3 or 4 or a functional variant or homolog thereof. More preferably, the nucleic acid sequence is operably linked to a regulatory sequence, wherein the regulatory sequence is selected from a constitutive promoter or a tissue-specific promoter.

Also provided is a vector comprising the nucleic acid construct described above, as well as a host cell comprising the nucleic acid construct.

In another aspect of the invention, there is provided the use of the nucleic acid construct or vector described above to increase seed yield.

In a final aspect of the invention there is provided a method of producing a food or feed composition, the method comprising
a. producing a plant wherein the activity of an AAP polypeptide is increased using the method described above;
b. obtaining a seed from said plant; and
c. producing a food or feed composition from said seed.

In one embodiment, the plant is a crop plant. In a further embodiment, the crop plant is selected from rice, maize, wheat, soybean, barley, cannabis, pennycress and Brassica. In a preferred embodiment, the plant part is a seed.

In a further aspect of the invention, there is provided a plant or plant progeny obtained or obtainable by any of the methods described above. In another embodiment, there is provided a seed obtained or obtainable by the plants or methods described herein, as well as progeny obtained from those plants and subsequent seeds obtained from the plants.

In a further aspect of the invention, there is provided a method of increasing free amino acid and/or protein content in a plant comprising increasing the activity of amino acid permease (AAP). Preferably, free amino acid and/or protein content is increased in the seed or grain of said plant. In one embodiment, the method comprises increasing the expression and/or activity of AAP8, wherein the amino acid sequence of AAP8 comprises a sequence as defined in SEQ ID NO: 2, 3 or 4 or a functional variant or homologue thereof.

### DESCRIPTION OF THE FIGURES

The invention is further described in the following non-limiting figures:

FIG. 1 shows that the NIL-SSW1^{Cvi} produces large seeds. (A) Mature seeds of Ler (left) and NIL-SSW1^{Cvi} (right). (B) Mature embryos of Ler (left) and NIL-SSW1^{Cvi} (right). (C) and (D) Ten-day-old seedlings of Ler (C) and NIL-SSW1^{Cvi} (D). (E) and (F) The average area of Ler and NIL-SSW1^{Cvi} seeds from main stems (E) and branches (F). (G) to (I) Length, width and weight of Ler and NIL-SSW1^{Cvi} seeds from main stems.

(J) The average cotyledon area of 10-d-old seedlings of Ler and NIL-SSW1^{Cvi}. Values in (E) to (J) are given as mean±SE relative to the wild-type values, set at 100%. **, P<0.01 compared with the wild type (Student's t test). Bars=0.5 mm in (A), 0.1 mm in (B), 1 mm in (C) and (D).

FIG. 2 shows that SSW1 regulates cell proliferation in the maternal integuments.

(A) Seed area of Ler/Ler F₁, SSW1^{Cvi}/SSW1^{Cvi} F₁, Ler/SSW1^{Cvi} F₁ and SSW1^{Cvi}/Ler F₁. (B) Seed area of Ler/Ler F₂, SSW1^{Cvi}/SSW1^{Cvi} F₂, Ler/SSW1^{Cvi} F₂ and SSW1^{Cvi}/Ler F₂. (C) and (D) The mature ovules of Ler (C) and SSW1^{Cvi} (D). (E) and (F) The seeds of Ler (E) and SSW1^{Cvi} (F) at 6 DAP (days after pollination). (G) The outer integument length of Ler and SSW1^{Cvi} at 0, 6, 8 DAP. (H) The number of cells in the outer integuments of Ler and SSW1^{Cvi} at 0, 6, 8 DAP. (I) The length of cells in the outer integuments of Ler and SSW1^{Cvi} at 0, 6, 8 DAP. Values in (A) and (B) are given as mean±SE relative to respective wildtype values, set at 100%. Values in (G) to (I) are given as mean±SE. **, P<0.01 compared with the wildtype by Student's t test. Bar=100 μm in (C) to (F).

FIG. 3 shows that the SSW1/AAP8 gene encodes the amino acid permease 8 (AAP8). (A) and (B) The AAP8 gene was mapped into the interval between markers Cvi-m33 and Cvi-m51 by using an F₂ population of 10,048 individuals and progeny tests. The mapping region contains four genes. (C) Quantitative real-time PCR analysis show expression of At1g10010, At1g10020, At1g10030 and At1g10040 in the 2nd to 5th siliques from Ler and NIL-SSW1^{Cvi} main stems. (D) The structure of the SSW1/AAP8 gene. The red color marked substitutions can cause amino acid change.

(E) Distribution of Arabidopsis accessions with SSW1^{Ler}, SSW1^{Cvi} and SSW1^{Col-0} types, respectively. (F) The schematic diagram of the SSW1/AAP8 protein. Amino acid substitutions are marked as Ler/SSW1^{Cvi}. For example, A/V means alanine in Ler and valine in Cvi and NIL-SSW1^{Cvi}. “Aa_trans motif” represents “amino acid transporter” in Pfam database (PF01490). (G) Seed area and weight of Ler, NIL-SSW1^{Cvi}, gSSW1^{Cvi}-COM #6 (homozygous), gSSW1^{Cvi}-COM #9 (homozygous) and gSSW1^{Cvi}-COM #16 (homozygous). (H) The expression levels of AAP8 in Col-0, aap8-1, and aap8-101.

(I) Seed area and weight of Col-0, aap8-1, and aap8-101. (J) Seed area of Col-0, aap8-1, gSSW1^{Cvi}-COM; aap8-1 #1 (homozygous), gSSW1^{Cvi}-COM; aap8-1 #2 (homozygous) and gSSW1^{Cvi}-COM; aap8-1 #3 (homozygous). Values in (C) and (H) are given as mean±SE. Values in (G) (I) and (J) are given as mean±SE relative to the respective wild-type values, set at 100%. **, P<0.01 compared with the wild-type (Student's t test).

FIG. 4 shows that natural variation in SSW1/AAP8 influences amino acid permease activity. (A) Schematic representation of SSW1 harboring different natural allelic variations and mutations. Three types of natural allelic variations in SSW1/AAP8 (SSW1^{Ler}, SSW1^{Cvi}, and SSW1^{Col-0}) were shown. (B) Growth of 22Δ8AA transformed with SSW1 harboring different amino acid variations or mutations in nitrogen free medium supplemented with 1 mM ASP.

Values in (B) are given as mean±SE.

FIG. 5 shows that the SSW1^{Cvi} natural allele seeds contain more free amino acids and storage proteins. (A) Comparison of free amino acid content of young siliques (2-5 days after pollination) of Ler and NIL-SSW1^{Cvi}. (B) Comparison of free amino acid content of dry seeds of Ler and NIL-SSW1^{Cvi}. (C) Analysis of total free amino acid content of young siliques (2-5 days after pollination, left) and dry seeds (right) of Ler and NIL-SSW1^{Cvi}. (D) Analysis of soluble seed proteins by SDS-PAGE gel.

Values in (A) and (B) are given as mean±SE. Values in (C) is given as mean±SE relative to the respective wild-type values, set at 100%. **, P<0.01 and *, P<0.05 compared to the wildtype by Student's t test. (E) Quantification of the soluble seed proteins in Ler was relative to that in NIL-SSW1^{Cvi} from (D). The ratio values of soluble seed proteins in Ler were set at 1. Values for soluble seed proteins in NIL-SSW1^{Cvi} are given as mean±SD (n=3). **P<0.01 compared with the value for Ler by Student's t-test. Values in (A) and (B) are given as mean±SE. Values in (C) and (E) is given as mean±SE relative to the respective wild-type values, set at 100%. **, P<0.01 and *, P<0.05 compared to the wildtype by Student's t test.

FIG. 6 shows the genetic interactions between AAP8/SSW1 and AAP1. (A) The AAP1 gene structure. The T-DNA insertion site in aap1-101 was shown. Arrows indicate the priming site of primes used for Real-time PCR in (C). (B) The AAP1 protein structure. (C) The expression levels of AAP1 in Col-0 and aap1-101. (D) Seed area of Col-0, aap8-1, aap1-101, and aap8-1 aap1-101. (E) Seed weight of Col-0, aap8-1, aap1-101, and aap8-1 aap1-101. (F) A model for AAP8 regulation in amino acid permease activity between different natural allelic variations/two Arabidopsis accessions. This includes transporters involved in amino acid uptake into the endosperm (AAP8/SSW1) and embryo (AAP1). Different arrow shapes represent that amino acids are transported by different transporters (SSW1/AAP8 and AAP1). Thicker arrows represent higher amino acid permease activity. The amino acid V410A is mainly responsible for the activity differences between SSW1^{Cvi} and SSW1^{Ler}. Values in (D) to (E) are given as mean±SE relative to the respective wild-type values, set at 100%. **, P<0.01 compared with their respective control (Student's t test).

FIG. 7 shows the seed area and weight of Ler, LCN1-3-3 and Cvi. Values are given as mean±SE relative to Ler, set at 100%.

FIG. 8 shows the seed area of gSSW1Ler-COM #and gSSW1^{Cvi}-COM #transgenic lines. Values are given as mean±SE relative to the respective wild-type values, set at 100%. **, P<0.01 compared with the wild-type (Student's t test).

FIG. 9 shows that the seed size of aap8-1 is controlled maternally. (A) Seed area of Col-0/Col-0 F1, aap8-1/aap8-1 F1, Col-0/aap8-1 F1 and aap8-1/Col-0 F1. (B) Seed area of Col-0/Col-0 F2, aap8-1/aap8-1 F2, Col-0/aap8-1 F2 and aap8-1/Col-0 F2. (C) The outer integument length of Col-0 and aap8-1 at 0, 6, 8 DAP. (D) The number of cells in the outer integuments of Col-0 and aap8-1 at 0, 6, 8 DAP. (E) The length of cells in the outer integuments of Col-0 and aap8-1 at 0, 6, 8 DAP. Values in (A) and (B) are given as mean±SE relative to the respective wild-type values, set at 100%. Values in (C) to (E) are given as mean±SE. **, P<0.01 compared with the wild-type (Student's t test).

FIG. 10 shows the gSSW1Cvi-COM #transgene lines contain more storage proteins. (a) The contents of soluble seed proteins by SDS-PAGE of three different gSSW1Cvi-COM lines (homozygous) and their individual Ler counterparts. We obtained Ler #1 (Lane A) and gSSW1Cvi-COM #9 (Lane B) seeds, Ler #2 (Lane C) and gSSW1Cvi-COM #5 (Lane D) seeds, Ler #3 (Lane E) and gSSW1Cvi-COM #15 (Lane F) seeds from their respective heterozygous maternal lines. (b) Quantification of the soluble seed proteins in different gSSW1^{Cvi}-COM transgene lines was relative to that in Ler from (A) and Supplemental FIG. 14B. The ratio values of soluble seed proteins in Ler were set at 1. Values for soluble seed proteins in gSSW1^{Cvi}-COM are given as mean±SD (n=3). **P<0.01 compared with the value for Ler by Student's t-test.

FIG. 11 is a list of SNPs in the SSW1 gene between Ler and Cvi.

FIG. 12 shows a table of point mutations at the homologous sequence position to At AAP8 A410. Homologous species listed are Rice, Maize, Barley, Soy Bean, Wheat and Brassica.

### DETAILED DESCRIPTION OF THE INVENTION

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous.

The practice of the present invention will employ, unless otherwise indicated, conventional techniques of botany, microbiology, tissue culture, molecular biology, chemistry, biochemistry and recombinant DNA technology, bioinformatics which are within the skill of the art. Such techniques are explained fully in the literature.

The terms “seed” and “grain” as used herein can be used interchangeably.

As used herein, the words “nucleic acid”, “nucleic acid sequence”, “nucleotide”, “nucleic acid molecule” or “polynucleotide” are intended to include DNA molecules (e.g., cDNA or genomic DNA), RNA molecules (e.g., mRNA), natural occurring, mutated, synthetic DNA or RNA molecules, and analogs of the DNA or RNA generated using nucleotide analogs. It can be single-stranded or double-stranded. Such nucleic acids or polynucleotides include, but are not limited to, coding sequences of structural genes, anti-sense sequences, and non-coding regulatory sequences that do not encode mRNAs or protein products. These terms also encompass a gene. The term “gene” or “gene sequence” is used broadly to refer to a DNA nucleic acid associated with a biological function. Thus, genes may include introns and exons as in the genomic sequence, or may comprise only a coding sequence as in cDNAs, and/or may include cDNAs in combination with regulatory sequences.

The terms “polypeptide” and “protein” are used interchangeably herein and refer to amino acids in a polymeric form of any length, linked together by peptide bonds.

The aspects of the invention involve recombination DNA technology and exclude embodiments that are solely based on generating plants by traditional breeding methods.

For the purposes of the invention, a “genetically altered” or “mutant” plant is a plant that has been genetically altered compared to the naturally occurring wild type (WT) plant. In one embodiment, a mutant plant is a plant that has been altered compared to the naturally occurring wild type (WT) plant using a mutagenesis method, such as the mutagenesis methods described herein. In one embodiment, the mutagenesis method is targeted genome modification or genome editing. In one embodiment, the plant genome has been altered compared to wild type sequences using a mutagenesis method. In one example, mutations can be used to insert an AAP gene sequence to increase the activity of AAP. In one example, the AAP sequence is operably linked to an endogenous promoter. Such plants have an altered phenotype as described herein, such as an increased seed yield. Therefore, in this example, increased seed yield is conferred by the presence of an altered plant genome and is not conferred by the presence of transgenes expressed in the plant.

Methods of Increasing Seed Yield

In a first aspect of the invention, there is provided a method of increasing seed yield in a plant, the method comprising increasing the activity of an amino acid permease (AAP) in a plant.

Seed size and weight are the main components contributing to seed yield, however, in one embodiment, the increase in seed yield comprises an increase in at least one yield component trait such as seed length and seed width, including average seed length, width and/or area, seed weight (single seed or thousand grain weight), overall seed yield per plant, and/or seed quality (preferably an increase in storage proteins and/or free amino acids) per seed. In particular, the inventors have found that increasing the activity of an AAP increases at least one of seed weight, seed size and seed quality. Preferably, increasing the activity of an AAP increases seed weight, seed size and seed quality.

The terms “increase”, “improve” or “enhance” as used herein are interchangeably. In one embodiment, seed yield, and preferably seed weight, seed size (e.g. seed length and/or width and/or seed area) and/or seed quality is increased by at least 2%, 3%, 4%, 5%, 6%, 7%, 8%, 9%, 10% 11%, 12%, 13%, 14%, 15%, 16%, 17%, 18%, 19%, 20%, 30%, 40% or 50% compared to a control plant. Preferably, seed yield is increased by at least 5%, more preferably between 5 and 30% compared to a control plant. In one embodiment, total free amino acid content in the seeds increased by between 5 and 50%, more preferably between 10 and 40% compared to a control plant.

Thus, according to the invention, seed yield can be measured by assessing one or more of seed weight, seed size and/or protein (or free amino acid) content in the plant. Yield is increased relative to control plants. The skilled person would be able to measure any of the above seed yield parameters using known techniques in the art. Protein or amino acid levels may be measured using standard techniques in the art, such as, but not limited to, infrared radiation analyses and use of the Bradford assay.

Accordingly, in another aspect of the invention, there is provided a method of increasing free amino acid and/or protein content in a plant comprising increasing the activity of amino acid permease (AAP). Preferably, free amino acid and/or protein content is increased in the seed or grain of said plant.

Amino acid permease or AAP is a membrane transport protein that transports amino acids into the cell. By “increase activity” is meant that the ability of the permease to transport amino acids, an in particular, aspartate, into a cell is increased, particularly when compared to a wild-type or control plant. FIG. 4 shows one method to measure the activity of an amino acid permease, but other methods would be well known to the skilled person.

In one embodiment, the AAP is AAP8 (which is also referred to herein as SSW1). More preferably AAP8 comprises or consists of an amino acid sequence as defined in any one of SEQ ID NO: 1 to 4 or a functional variant or homologue thereof. In a further preferred embodiment, AAP8 comprises or consists of a nucleic acid sequence as defined in any one of SEQ ID NO: 5 to 8 or a functional variant or homologue thereof.

In one embodiment, the activity of an AAP is increased by introducing and expressing a nucleic acid construct where the nucleic acid construct comprises a nucleic acid sequence encoding an AAP8 polypeptide as defined in SEQ ID NO: 2 (the Cvi allele) or 3 (the Col-0 allele) or 4 or a functional variant or homolog thereof. In a further embodiment, the nucleic acid construct comprises a nucleic acid sequence comprising or consisting of a nucleic acid sequence as defined in SEQ ID NO: 6, 7 or 8 or functional variant or homolog thereof.

In a preferred embodiment, the nucleic acid sequence is operably linked to a regulatory sequence. Accordingly, in one embodiment, the nucleic acid sequence may be expressed using a regulatory sequence that drives overexpression. Overexpression according to the invention means that the transgene is expressed or is expressed at a level that is higher than the expression of the endogenous AAP gene whose expression is driven by its endogenous counterpart. In one embodiment, the nucleic acid and regulatory sequence are from the same plant family. In another embodiment, the nucleic acid and regulatory sequence are from a different plant family, genus or species—for example, AtAAP8 is expressed in a plant that is not Arabidopsis.

In one embodiment, the regulatory sequence is a promoter. The term “promoter” typically refers to a nucleic acid control sequence located upstream from the transcriptional start of a gene and which is involved in the binding of RNA polymerase and other proteins, thereby directing transcription of an operably linked nucleic acid. Encompassed by the aforementioned terms are transcriptional regulatory sequences derived from a classical eukaryotic genomic gene (including the TATA box which is required for accurate transcription initiation, with or without a CCAAT box sequence) and additional regulatory elements (i.e. upstream activating sequences, enhancers and silencers) which alter gene expression in response to developmental and/or external stimuli, or in a tissue-specific manner. Also included within the term is a transcriptional regulatory sequence of a classical prokaryotic gene, in which case it may include a −35 box sequence and/or −10 box transcriptional regulatory sequences.

A “plant promoter” comprises regulatory elements that mediate the expression of a coding sequence segment in plant cells. The promoters upstream of the nucleotide sequences useful in the nucleic acid constructs described herein can also be modified by one or more nucleotide substitution(s), insertion(s) and/or deletion(s) without interfering with the functionality or activity of either the promoters, the open reading frame (ORF) or the 3′-regulatory region such as terminators or other 3′ regulatory regions which are located away from the ORF. It is furthermore possible that the activity of the promoter is increased by modification of their sequence, or that they are replaced completely by more active promoters, even promoters from heterologous organisms. For expression in plants, the AAP nucleic acid sequence is, as described above, preferably linked operably to or comprises a suitable promoter, which expresses the gene at the right point in time and with the required spatial expression pattern.

In one embodiment, overexpression may be driven by a constitutive promoter. A “constitutive promoter” refers to a promoter that is transcriptionally active during most, but not necessarily all, phases of growth and development and under most environmental conditions, in at least one cell, tissue or organ. Examples of constitutive promoters include the cauliflower mosaic virus promoter (CaMV35S or 19S), rice actin promoter, ubiquitin promoter, rubisco small subunit, maize or alfalfa H3 histone, OCS, SAD1 or 2, GOS2 or any promoter that gives enhanced expression

In an alternative embodiment, the promoter is a tissue-specific promoter. Tissue specific promoters are transcriptional control elements that are only active in particular cells or tissues at specific times during plant development. In one example, the tissue-specific promoter is a seed coat-specific promoter, for example, the MUM4 (Mucilage-modified4)0.3Pro, as defined in, for example, SEQ ID NO: 169 or a functional variant thereof.

The term “operably linked” as used herein refers to a functional linkage between the promoter sequence and the gene of interest, such that the promoter sequence is able to initiate transcription of the gene of interest.

In one embodiment, the progeny plant is stably transformed with the nucleic acid construct described herein and comprises the exogenous polynucleotide, which is heritably maintained in the plant cell. The method may include steps to verify that the construct is stably integrated. The method may also comprise the additional step of collecting seeds from the selected progeny plant.

In an alternative embodiment, the method comprises introducing at least one mutation into the plant genome to increase the activity of an AAP, as defined herein.

In one embodiment, the mutation is the insertion of at least one or more additional copy of an AAP with increased activity as defined herein. For example, the mutation may comprise the insertion of at least one or more additional copy of a nucleic acid encoding an AAP8 polypeptide as defined in SEQ ID NO: 2 (Cvi allele) or 3 (Col-0 allele) or 4 or a functional variant or homolog thereof, such that the sequence is operably linked to a regulatory sequence.

In another embodiment, the method comprises introducing at least one mutation into at least one AAP gene. Preferably, the method comprises introducing at least one mutation into the, preferably endogenous, nucleic acid sequence encoding an AAP polypeptide. As used herein, the term “endogenous” may refer to the native or natural sequence in the plant genome. In one embodiment, the endogenous amino acid sequence of AAP8 is defined in SEQ ID NO: 1 (Ler allele) or a functional variant or homologue thereof. More preferably, the nucleic acid sequence encoding an AAP comprises or consists of SEQ ID NO: 5 (genomic sequence of the Ler allele) or a functional variant or homologue thereof.

The term “functional variant of a nucleic acid sequence” as used herein with reference to any of the sequences described herein refers to a variant gene or amino acid sequence or part of the gene or amino acid sequence that retains the biological function of the full non-variant sequence. A functional variant also comprises a variant of the gene of interest that has sequence alterations that do not affect function, for example in non-conserved residues. Also encompassed is a variant that is substantially identical, i.e. has only some sequence variations, for example in non-conserved residues, compared to the wild type sequences as shown herein and is biologically active. Alterations in a nucleic acid sequence which result in the production of a different amino acid at a given site that do not affect the functional properties of the encoded polypeptide are well known in the art. For example, a codon for the amino acid alanine, a hydrophobic amino acid, may be substituted by a codon encoding another less hydrophobic residue, such as glycine, or a more hydrophobic residue, such as valine, leucine, or isoleucine. Similarly, changes which result in substitution of one negatively charged residue for another, such as aspartic acid for glutamic acid, or one positively charged residue for another, such as lysine for arginine, can also be expected to produce a functionally equivalent product. Nucleotide changes which result in alteration of the N-terminal and C-terminal portions of the polypeptide molecule would also not be expected to alter the activity of the polypeptide. Each of the proposed modifications is well within the routine skill in the art, as is determination of retention of biological activity of the encoded products.

In one embodiment, a functional variant has at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the non-variant nucleic acid or amino acid sequence.

The term homolog, as used herein, also designates an AAP8 gene orthologue from other plant species. A homolog may have, in increasing order of preference, at least 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the amino acid represented by any of SEQ ID NO: 1 to 4 or to the nucleic acid sequences as shown by SEQ ID NOs: 5 to 8. In one embodiment, overall sequence identity is at least 37%. In one embodiment, overall sequence identity is at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99%, most preferably 90%, 91%, 92%, 93%, 94%, 95%, 96%, 97%, 98%, or at least 99%. Functional variants of an AAP8 homolog are also within the scope of the invention.

Examples of AAP8 homologues are described in SEQ ID Nos 9 to 166. Specifically, the amino acid sequence of AAP8 homolog may be selected from one of SEQ ID Nos 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 165 or a functional variant thereof. In a further embodiment, the nucleic acid sequence of an AAP8 homolog may be selected from SEQ ID Nos 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 146, 138, 140, 142, 144, 146, 148, 150, 152, 154, 156, 158, 160, 162, 164 or 166 or a functional variant thereof.

In one embodiment, where the homolog is rice, the amino acid sequence of the AAP8 homolog comprises or consists of SEQ ID NO: 9 or 13 or a functional variant thereof, and the nucleic acid sequence of the AAP8 homolog comprises or consists of SEQ ID NO: 10 or 14 or a functional variant thereof.

In a further embodiment, where the homolog is soybean, the amino acid sequence of the AAP8 homolog comprises or consists of SEQ ID NO: 31 or a functional variant thereof, and the nucleic acid sequence of the AAP8 homolog comprises or consists of SEQ ID NO: 32 or a functional variant thereof.

In a further embodiment, where the homolog is maize, the amino acid sequence of the AAP8 homolog comprises or consists of SEQ ID NO: 63 or a functional variant thereof, and the nucleic acid sequence of the AAP8 homolog comprises or consists of SEQ ID NO: 64 or a functional variant thereof.

In a further embodiment, where the homolog is B. napus, the amino acid sequence of the AAP8 homolog comprises or consists of SEQ ID NO: 123 or a functional variant thereof, and the nucleic acid sequence of the AAP8 homolog comprises or consists of SEQ ID NO: 124 or a functional variant thereof.

In a further embodiment, where the homolog is B. rapa, the amino acid sequence of the AAP8 homolog comprises or consists of SEQ ID NO: 139, 141 or 143 or a functional variant thereof, and the nucleic acid sequence of the AAP8 homolog comprises or consists of SEQ ID NO: 140, 142 or 144 or a functional variant thereof.

In a further embodiment, where the homolog is B. oleracea, the amino acid sequence of the AAP8 homolog comprises or consists of SEQ ID NO: 157 or a functional variant thereof, and the nucleic acid sequence of the AAP8 homolog comprises or consists of SEQ ID NO: 158 or a functional variant thereof.

In a further embodiment, where the homolog is barley, the amino acid sequence of the AAP8 homolog comprises or consists of SEQ ID NO: 131 or a functional variant thereof, and the nucleic acid sequence of the AAP8 homolog comprises or consists of SEQ ID NO: 132 or a functional variant thereof.

In a further embodiment, where the homolog is wheat, the amino acid sequence of the AAP8 homolog comprises or consists of SEQ ID NO: 135 or 136 or a functional variant thereof, and the nucleic acid sequence of the AAP8 homolog comprises or consists of SEQ ID NO: 138 or 140 or a functional variant thereof.

In a further embodiment, the AAP polypeptide of the invention comprises the following conserved motif. Preferably, the at least one mutation is in at least one of these residues, more preferably in the first residue (i.e. the X residue):

(SEQ ID NO: 167)
XFWPLTVY

wherein X is any amino acid, but preferably is an A, S or G.

In an alternative embodiment, the AAP polypeptide comprises an amino acid transporter motif (referred to herein as “Aa_trans motif”) as defined below or a functional variant thereof and preferably, the at least one mutation is in the amino acid transporter motif.

Aa_trans motif:
SEQ ID NO: 168
RTGTFWTASAHIITAVIGSGVLSLAWAIAQLGWVAGTTVLVAFAIITYYT
STLLADCYRSPDSITGTRNYNYMGVVRSYLGGKKVQLCGVAQYVNLVGVT
IGYTITASISLVAIGKSNCYHDKGHKAKCSVSNYPYMAAFGIVQIILSQL
PNFHKLSFLSIIAAVMSFSYASIGIGLAIATVASGKIGKTELTGTVIGVD
VTASEKVWKLFQAIGDIAFSYAFTTILIEIQDTLRSSPPENKVMKRASLA
GVSTTTVFYILCGCIGYAAFGNQAPGDFLTDFGFYEPYWLIDFANACIAL
HLIGAYQVYAQPFFQFVEENCNKKWPQSNFINKEYSSKVPLLGKCRVNLF
RLVWRTCYVVLTTFVAMIFPFFNAILGLLGAFVFWPLTVYFPVAMHIAQA
KVKKYSRRWLALNLLVLVCLIVSALAAVGSIIGLI

Accordingly, in one embodiment, there is provided a method of increasing seed yield in a plant as described herein, the method comprising increasing the activity of an AAP polypeptide as described herein, wherein the AAP comprises or consists of one of the following sequences:
a. a nucleic acid sequence encoding an AAP polypeptide as defined in SEQ ID NO: 2, 3, 4, 9, 11, 13, 15, 17, 19, 21, 23, 25, 27, 29, 31, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117, 119, 121, 123, 125, 127, 129, 131, 133, 135, 137, 139, 141, 143, 145, 147, 149, 151, 153, 155, 157, 159, 161, 163 or 165 or a functional variant thereof; or
b. a nucleic acid sequence as defined in SEQ ID NO: 6, 7, 8, 10, 12, 14, 16, 18, 20, 22, 24, 26, 28, 30, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 118, 120, 122, 124, 126, 128, 130, 132, 134, 146, 138, 140, 142, 144, 146, 148, 150, 152, 154, 156, 158, 160, 162, 164 or 166 or a functional variant thereof; or
c. a nucleic acid sequence encoding an AAP polypeptide, wherein the polypeptide comprises an amino acid transporter motif as defined in SEQ ID NO: 168 or a variant thereof, wherein the variant has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to SEQ ID NO: 167; or
d. a nucleic acid sequence encoding an AAP polypeptide, wherein the polypeptide comprises the sequence defined in SEQ ID NO: 168 or a variant thereof, wherein the variant has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to SEQ ID NO: 168;
wherein the functional variant has at least 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or at least 99% overall sequence identity to the sequences in (a) or (b) and/or wherein the functional variant encodes an AAP polypeptide and is capable of binding under stringent hybridisation conditions as defined herein to one of the sequences in (a), (b), (c) or (d).

In one embodiment, the mutation in the nucleic acid sequence encoding an AAP polypeptide may be selected from one of the following mutation types:
1. a “missense mutation”, which is a change in the nucleic acid sequence (e.g. a change in one or more nucleotides) that results in the substitution of one amino acid for another amino acid (also known as a nonsynonymous substitution);
2. an “insertion mutation” of one or more nucleotides or one or more amino acids, due to one or more codons having been added in the coding sequence of the nucleic acid;
3. a “deletion mutation” of one or more nucleotides or of one or more amino acids, due to one or more codons having been deleted in the coding sequence of the nucleic acid;

In one embodiment the mutation is a missense mutation (nonsynonymous substitution).

In one embodiment, the one or more mutations in the AAP nucleic acid sequence results in an amino acid substitution at position 410 in SEQ ID NO: 1 or a homologous position in a homologous sequence. Preferably, said mutation arises from a substitution of one or more nucleotides in the nucleic acid sequence of AAP8. In one embodiment, the mutation is at position 2635 of SEQ ID NO: 5 or a homologous position in a homologous sequence.

In a further embodiment, the method may comprise introducing one or more additional mutations, preferably at position 277 and/or 374 of SEQ ID NO: 1 or a homologous position in a homologous sequence.

In a further embodiment, the nonsense mutation in the nucleic acid sequence causes a substitution of one amino acid for another in the resulting amino acid sequence. In one embodiment, the mutation is the substitution of one hydrophobic amino acid for another hydrophobic amino acid. For example, the substituted residue may be selected from alanine, isoleucine, leucine, methionine, phenylalanine, tryptophan, tyrosine and valine. More preferably the substituted residue is selected from valine, isoleucine and alanine. Most preferably the substituted residue is alanine.

“By at least one mutation” is meant that where the AAP gene is present as more than one copy or homologue (with the same or slightly different sequence) there is at least one mutation in at least one gene. Preferably all genes are mutated.

The skilled person would understand that suitable homologues and the homologous positions in these sequences can be identified by sequence comparisons and identifications of conserved domains. There are predictors in the art that can be used to identify such sequences. The function of the homologue can be identified as described herein and a skilled person would thus be able to confirm the function. Homologous positions can thus be determined by performing sequence alignments once the homologous sequence has been identified. For example, AAP8 homologues can be identified using a BLAST search of the plant genome of interest using the Arabidopsis AAP8 as a query.

Identification of the homologous position in any AAP8 homologous sequence can be performed by making a multiple sequence alignment of the candidate sequence with the Arabidopsis AAP8. In particular, the conserved amino acid transporter motif can be aligned using any known multiple sequence alignment program (e.g. DNAMAN) with the corresponding motif in a candidate homologous sequence to identify the homologous position.

Thus, the nucleotide sequences of the invention and described herein can also be used to isolate corresponding sequences from other organisms, particularly other plants, for example crop plants. In this manner, methods such as PCR, hybridization, and the like can be used to identify such sequences based on their sequence homology to the sequences described herein. Topology of the sequences and the characteristic domain structure can also be considered when identifying and isolating homologs. Sequences may be isolated based on their sequence identity to the entire sequence or to fragments thereof. In hybridization techniques, all or part of a known nucleotide sequence is used as a probe that selectively hybridizes to other corresponding nucleotide sequences present in a population of cloned genomic DNA fragments or cDNA fragments (i.e., genomic or cDNA libraries) from a chosen plant. The hybridization probes may be genomic DNA fragments, cDNA fragments, RNA fragments, or other oligonucleotides, and may be labelled with a detectable group, or any other detectable marker. Methods for preparation of probes for hybridization and for construction of cDNA and genomic libraries are generally known in the art and are disclosed in Sambrook, et al., (1989) Molecular Cloning: A Library Manual (2d ed., Cold Spring Harbor Laboratory Press, Plainview, N.Y.).

In one embodiment, the homologous position and the homologous amino acid and nucleotide sequence of AtAAP8 is selected from one of the positions and amino acid and nucleotide sequences in the table of FIG. 12.

In one embodiment, the mutation is introduced using mutagenesis (i.e. any site-directed mutagenesis method) or targeted genome editing. That is, in one embodiment, the invention relates to a method and plant that has been generated by genetic engineering methods as described above, and does not encompass naturally occurring varieties.

Targeted genome modification or targeted genome editing is a genome engineering technique that uses targeted DNA double-strand breaks (DSBs) to stimulate genome editing through homologous recombination (HR)-mediated recombination events. In one embodiment, the mutation is introduced using ZFNs, TALENs or CRISPR/Cas9.

In a preferred embodiment, the targeted genome editing technique is CRISPR. The use of this technology in genome editing is well described in the art, for example in U.S. Pat. No. 8,697,359 and references cited herein. In short, CRISPR is a microbial nuclease system involved in defence against invading phages and plasmids. CRISPR loci in microbial hosts contain a combination of CRISPR-associated (Cas) genes as well as non-coding RNA elements capable of programming the specificity of the CRISPR-mediated nucleic acid cleavage (sgRNA). Three types (I-III) of CRISPR systems have been identified across a wide range of bacterial hosts. One key feature of each CRISPR locus is the presence of an array of repetitive sequences (direct repeats) interspaced by short stretches of non-repetitive sequences (spacers). The non-coding CRISPR array is transcribed and cleaved within direct repeats into short crRNAs containing individual spacer sequences, which direct Cas nucleases to the target site (protospacer). The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Watson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer.

One major advantage of the CRISPR-Cas9 system, as compared to conventional gene targeting and other programmable endonucleases is the ease of multiplexing, where multiple positions or sites on genes can be mutated simultaneously simply by using multiple sgRNAs each targeting a different site. In addition, where two sgRNAs are used flanking a genomic region, the intervening section can be deleted or inverted (Wiles et al., 2015). In the present invention, multiple sgRNAs can be used to simultaneously introduce two or more mutations, for example, the specific mutations described above, into the AAP8 gene. In this embodiment, self-cleaving RNAs or cleavable RNA molecules, such as csy4, ribozyme or tRNA sequences can be used to process a single construct into multiple sgRNAs.

Cas9 is thus the hallmark protein of the type II CRISPR-Cas system, and is a large monomeric DNA nuclease guided to a DNA target sequence adjacent to the PAM (protospacer adjacent motif) sequence motif by a complex of two noncoding RNAs: CRISPR RNA (crRNA) and trans-activating crRNA (tracrRNA). The Cas9 protein contains two nuclease domains homologous to RuvC and HNH nucleases. The HNH nuclease domain cleaves the complementary DNA strand whereas the RuvC-like domain cleaves the non-complementary strand and, as a result, a blunt cut is introduced in the target DNA. Heterologous expression of Cas9 together with an sgRNA can introduce site-specific double strand breaks (DSBs) into genomic DNA of live cells from various organisms. Codon optimized versions of Cas9, which is originally from the bacterium Streptococcus pyogenes, can also be used to increase efficiency. Cas9 orthologues may also be used, such as Staphylococcus aureus (SaCas9) or Streptococcus thermophiles (StCas9).

The single guide RNA (sgRNA) is the second component of the CRISPR/Cas system that forms a complex with the Cas9 nuclease. sgRNA is a synthetic RNA chimera created by fusing crRNA with tracrRNA. The sgRNA guide sequence located at its 5′ end confers DNA target specificity. Therefore, by modifying the guide sequence, it is possible to create sgRNAs with different target specificities. The canonical length of the guide sequence is 20 bp. In plants, sgRNAs have been expressed using plant RNA polymerase III promoters, such as U6 and U3. Accordingly, using techniques known in the art it is possible to design sgRNA molecules that targets the AAP gene as described herein. In one embodiment, the method comprises using any of the nucleic acid constructs or sgRNA molecules described herein.

Alternatively, Cpf1, which is another Cas protein, can be used as the endonuclease. Cpf1 differs from Cas9 in several ways: Cpf1 requires a T-rich PAM sequence (TTTV) for target recognition, Cpf1 does not require a tracrRNA, and as such only crRNA is required unlike Cas9 and the Cpf1-cleavage site is located distal and downstream to the PAM sequence in the protospacer sequence (Li et al., 2017). Furthermore, after identification of the PAM motif, Cpf1 introduces a sticky-end-like DNA double-stranded break with several nucleotides of overhang. As such, the CRISPR/CPf1 system consists of a Cpf1 enzyme and a crRNA.

Cas9 and Cpf1 expression plasmids for use in the methods of the invention can be constructed as described in the art. Cas9 or Cpf1 and the one or more sgRNA molecule may be delivered as separate or as a single construct. Where separate constructs are used for the delivery of the CRISPR enzyme (i.e. Cas9 or Cpf1) and the sgRNA molecule(s), the promoters used to drive expression of the CRISPR enzyme/sgRNA molecule may be the same or different. In one embodiment, RNA polymerase (Pol) II-dependent promoters can be used to drive expression of the CRISPR enzyme. In another embodiment, Pol III-dependent promoters, such as U6 or U3, can be used to drive expression of the sgRNA.

In one embodiment, the method uses a sgRNA to introduce a targeted SNP or mutation, in particular one of the substitutions described herein into a AAP gene. As explained below, the introduction of a template DNA strand, following a sgRNA-mediated snip in the double-stranded DNA, can be used to produce a specific targeted mutation (i.e. a SNP) in the gene using homology directed repair. In an alternative embodiment, at least one mutation may be introduced into the AAP gene, particularly at the positions described above, using any CRISPR technique known to the skilled person. In another example, sgRNA (for example, as described herein) can be used with a modified Cas9 protein, such as nickase Cas9 or nCas9 or a “dead” Cas9 (dCas9) or a Cas9 nickase (Cas9n) fused to a “Base Editor”—such as an enzyme, for example a deaminase such as cytidine deaminase, or TadA (tRNA adenosine deaminase) or ADAR or APOBEC. These enzymes are able to substitute one base for another. As a result no DNA is deleted, but a single substitution is made (Kim et al., 2017; Gaudelli et al. 2017).

The genome editing constructs may be introduced into a plant cell using any suitable method known to the skilled person. In an alternative embodiment, any of the nucleic acid constructs described herein may be first transcribed to form a preassembled Cas9-sgRNA ribonucleoprotein and then delivered to at least one plant cell using any of the above described methods, such as lipofection, electroporation, biolistic bombardment or microinjection.

Specific protocols for using the above-described CRISPR constructs would be well known to the skilled person. As one example, a suitable protocol is described in Ma & Liu (“CRISPR/Cas-based multiplex genome editing in monocot and dicot plants”) incorporated herein by reference.

Genetically Altered or Modified Plants and Methods of Producing Such Plants

In another aspect of the invention, there is provided a genetically altered plant, part thereof or plant cell, characterised in that the plant expresses an AAP polypeptide with increased activity. In a further embodiment, the plant is characterised by an increase in seed yield.

In one embodiment, the plant or plant cell may comprise a nucleic acid construct comprising a nucleic acid encoding an AAP8 polypeptide as defined in SEQ ID NO: 2, 3 or 4 or a functional variant or homolog thereof, as defined herein. In one embodiment, the construct is stably incorporated into the genome.

In an alternative embodiment, the plant may be produced by introducing a mutation into the plant genome by any of the above-described methods. In one embodiment, the mutation is the insertion of at least one additional copy of a nucleic acid encoding an AAP with increased activity as defined herein. For example, the mutation may comprise the insertion of at least one or more additional copy of a nucleic acid encoding an AAP8 polypeptide as defined in SEQ ID NO: 2 (Cvi allele) or 3 (Col-0 allele) or 4 or a functional variant or homolog thereof, such that the sequence is operably linked to a regulatory sequence. In an alternative embodiment, the mutation is a substitution at position 410 of SEQ ID NO: 1 or at a homologous position in a homologous sequence, as defined herein. Preferably the mutation is introduced into at least one plant cell and a plant regenerated from the at least one mutated plant cell.

The terms “introduction”, “transfection” or “transformation” as referred to herein encompass the transfer of an exogenous polynucleotide or construct (such as a nucleic acid construct or a genome editing construct as described herein) into a host cell, irrespective of the method used for transfer. Plant tissue capable of subsequent clonal propagation, whether by organogenesis or embryogenesis, may be transformed with a genetic construct of the present invention and a whole plant regenerated there from. The particular tissue chosen will vary depending on the clonal propagation systems available for, and best suited to, the particular species being transformed. Exemplary tissue targets include leaf disks, pollen, embryos, cotyledons, hypocotyls, megagametophytes, callus tissue, existing meristematic tissue (e.g., apical meristem, axillary buds, and root meristems), and induced meristem tissue (e.g., cotyledon meristem and hypocotyl meristem). The resulting transformed plant cell may then be used to regenerate a transformed plant in a manner known to persons skilled in the art.

The transfer of foreign genes into the genome of a plant is called transformation. Transformation of plants is now a routine technique in many species. Any of several transformation methods known to the skilled person may be used to introduce one or more genome editing constructs of interest into a suitable ancestor cell. The methods described for the transformation and regeneration of plants from plant tissues or plant cells may be utilized for transient or for stable transformation.

Transformation methods include the use of liposomes, electroporation, chemicals that increase free DNA uptake, injection of the DNA directly into the plant (microinjection), gene guns (or biolistic particle delivery systems (bioloistics)) as described in the examples, lipofection, transformation using viruses or pollen and microprojection. Methods may be selected from the calcium/polyethylene glycol method for protoplasts, ultrasound-mediated gene transfection, optical or laser transfection, transfection using silicon carbide fibers, electroporation of protoplasts, microinjection into plant material, DNA or RNA-coated particle bombardment, infection with (non-integrative) viruses and the like. Transgenic plants can also be produced via Agrobacterium tumefaciens mediated transformation, including but not limited to using the floral dip/Agrobacterium vacuum infiltration method as described in Clough & Bent (1998) and incorporated herein by reference.

Optionally, to select transformed plants, the plant material obtained in the transformation is, as a rule, subjected to selective conditions so that transformed plants can be distinguished from untransformed plants. For example, the seeds obtained in the above-described manner can be planted and, after an initial growing period, subjected to a suitable selection by spraying. A further possibility is growing the seeds, if appropriate after sterilization, on agar plates using a suitable selection agent so that only the transformed seeds can grow into plants. As described in the examples, a suitable marker can be bar-phosphinothricin or PPT. Alternatively, the transformed plants are screened for the presence of a selectable marker, such as, but not limited to, GFP, GUS (β-glucuronidase). Other examples would be readily known to the skilled person. Alternatively, no selection is performed, and the seeds obtained in the above-described manner are planted and grown and AAP activity levels measured at an appropriate time using standard techniques in the art. This alternative, which avoids the introduction of transgenes, is preferable to produce transgene-free plants.

Following DNA transfer and regeneration, putatively transformed plants may also be evaluated, for instance using PCR to detect the presence of the gene of interest, copy number and/or genomic organisation. Alternatively or additionally, integration and expression levels of the newly introduced DNA may be monitored using Southern, Northern and/or Western analysis, both techniques being well known to persons having ordinary skill in the art.

The method may further comprise selecting one or more mutated plants, preferably for further propagation. The selected plants may be propagated by a variety of means, such as by clonal propagation or classical breeding techniques. For example, a first generation (or T1) transformed plant may be selfed and homozygous second-generation (or T2) transformants selected, and the T2 plants may then further be propagated through classical breeding techniques. The generated transformed organisms may take a variety of forms. For example, they may be chimeras of transformed cells and non-transformed cells; clonal transformants (e.g., all cells transformed to contain the expression cassette); grafts of transformed and untransformed tissues (e.g., in plants, a transformed rootstock grafted to an untransformed scion).

In a further related aspect of the invention, there is also provided a method of obtaining a genetically modified plant as described herein, the method comprising
a. selecting a part of the plant;
b. transfecting at least one cell of the part of the plant of paragraph (a) with at least one nucleic acid construct as described herein or at least one sgRNA molecule as described herein, using the transfection or transformation techniques described above;
c. regenerating at least one plant derived from the transfected cell or cells;
d. selecting one or more plants obtained according to paragraph (c) that show increased activity of an AAP polypeptide.

In a further embodiment, the method also comprises the step of screening the genetically modified plant for the introduction of one or more additional copies of an AAP nucleic acid, as described herein, or for the introduction of one or more substitutions into the endogenous AAP genomic sequence. In one embodiment, the method comprises obtaining a DNA sample from a transformed plant and carrying out DNA amplification to detect one of the mutations described above. In a further embodiment, the methods comprise generating stable T2 plants preferably homozygous for the mutation.

A genetically altered plant of the present invention may also be obtained by transference of any of the sequences of the invention by crossing, e.g., using pollen of the genetically altered plant described herein to pollinate a wild-type or control plant, or pollinating the gynoecia of plants described herein with other pollen that does not contain at least one of the above-described mutations. The methods for obtaining the plant of the invention are not exclusively limited to those described in this paragraph; for example, genetic transformation of germ cells from the ear of wheat could be carried out as mentioned, but without having to regenerate a plant afterward.

In a further aspect of the invention there is provided a plant obtained or obtainable by the above-described methods. Also included in the scope of the invention is the progeny obtained from the plants.

The plant according to the various aspects of the invention may be a monocot or a dicot plant. A dicot plant may be selected from the families including, but not limited to Asteraceae, Brassicaceae (eg Brassica napus, Thlaspi arvense), Chenopodiaceae, Cucurbitaceae, Leguminosae (Caesalpiniaceae, Aesalpiniaceae Mimosaceae, Papilionaceae or Fabaceae), Malvaceae, Rosaceae or Solanaceae. For example, the plant may be selected from lettuce, sunflower, Arabidopsis, broccoli, spinach, water melon, squash, cabbage, tomato, potato, yam, capsicum, tobacco, cotton, okra, apple, rose, strawberry, alfalfa, bean, soybean, field (fava) bean, pea, lentil, peanut, chickpea, apricots, pears, peach, grape vine or citrus species.

A monocot plant may, for example, be selected from the families Arecaceae, Amaryllidaceae or Poaceae. For example, the plant may be a cereal crop, such as wheat, rice, barley, maize, oat, sorghum, rye, millet, buckwheat, turf grass, Italian rye grass, sugarcane or Festuca species, or a crop such as onion, leek, yam or banana.

Preferably, the plant is a crop plant. By crop plant is meant any plant which is grown on a commercial scale for human or animal consumption or use. Preferred plants are maize, wheat, rice, oilseed rape, cannabis, sorghum, soybean, pennycress, potato, tomato, grape, barley, pea, bean, field bean, lettuce, cotton, sugar cane, sugar beet, broccoli or other vegetable brassicas or poplar.

The term “plant” as used herein encompasses whole plants, ancestors and progeny of the plants and plant parts, including seeds, fruit, shoots, stems, leaves, roots (including tubers), flowers, tissues and organs, wherein each of the aforementioned comprise the nucleic acid construct as described herein. The term “plant” also encompasses plant cells, suspension cultures, callus tissue, embryos, meristematic regions, gametophytes, sporophytes, pollen and microspores, again wherein each of the aforementioned comprises the nucleic acid construct as described herein.

The invention also extends to harvestable parts of a plant of the invention as described herein, but not limited to seeds, leaves, fruits, flowers, stems, roots, rhizomes, tubers and bulbs. The aspects of the invention also extend to products derived, preferably directly derived, from a harvestable part of such a plant, such as dry pellets or powders, oil, fat and fatty acids, starch or proteins. Another product that may derived from the harvestable parts of the plant of the invention is biodiesel. The invention also relates to food products and food supplements comprising the plant of the invention or parts thereof. In one embodiment, the food products may be animal feed. In another aspect of the invention, there is provided a product derived from a plant as described herein or from a part thereof.

In a further aspect of the invention there is provided a method for producing a food or feed product with increased protein content, said method comprising
a. producing a plant wherein the activity of an AAP polypeptide, preferably AAP8 or homologue as described herein, is increased;
b. obtaining a seed from said plant;
c. producing a food or feed product from said seed.

In a preferred embodiment, the plant part or harvestable product is a seed. Therefore, in a further aspect of the invention, there is provided a seed produced from a genetically altered plant as described herein. In an alternative embodiment, the plant part is pollen, a propagule or progeny of the genetically altered plant described herein. Accordingly, in a further aspect of the invention there is provided pollen, a propagule or progeny of the genetically altered plant as described herein.

A control plant as used herein according to all of the aspects of the invention is a plant which has not been modified according to the methods of the invention. Accordingly, in one embodiment, the control plant does not have increased activity of an AAP polypeptide. In an alternative embodiment, the plant been genetically modified, as described above. In one embodiment, the control plant is a wild type plant. The control plant is typically of the same plant species, preferably having the same genetic background as the modified plant.

In another aspect of the invention, there is provided a nucleic acid construct comprising a nucleic acid sequence encoding a AAP8 polypeptide as defined in SEQ ID NO: 2 (the Cvi allele) or 3 (the Col-0 allele) or 4 or a functional variant or homolog thereof (as defined herein). In a further embodiment, the nucleic acid construct comprises a nucleic acid sequence comprising or consisting of a nucleic acid sequence as defined in SEQ ID NO: 6 or 7, or 8 or functional variant or homolog thereof. Preferably, the nucleic acid is operably linked to a regulatory sequence as defined herein.

In a further aspect of the invention, there is provided an isolated cell, preferably a plant cell or an Agrobacterium tumefaciens cell, expressing a nucleic acid construct as described herein. Furthermore, the invention also relates to a culture medium or kit comprising an isolated plant cell or an Agrobacterium tumefaciens cell expressing the nucleic acid construct described herein.

There is also provided the use of the nucleic acid construct described herein to increase seed yield.

Method of Screening Plants for Naturally Occurring High Levels of AAP Activity

In another aspect of the invention, there is provided a method for screening a population of plants and identifying and/or selecting a plant that has increased activity of at least one AAP polypeptide, wherein the method comprises detecting in the plant germplasm at least one polymorphism correlated with increased activity of an AAP polypeptide, as described herein. Preferably, said plant has an increased seed yield.

In one embodiment, the polymorphism is a substitution. In one specific embodiment, said polymorphism may comprise at least one substitution at position 2635 of SEQ ID NO: 5, 6, 7 or 8 or a homologous position in a homologous sequence, as described herein.

In a further embodiment, the method may further comprise detecting one or more additional polymorphisms, wherein preferably the one or more additional polymorphisms are selected from:
a substitution at position 2044 of SEQ ID NO: 5, 6, 7 or 8 or a homologous position in a homologous sequence; and/or
a substitution at position 2526 of SEQ ID NO: 5, 6, 7 or 8 or a homologous position in a homologous sequence.

Examples of homologous positions in a number of homologous sequences are shown in FIG. 12. Accordingly, in one embodiment, the at least one polymorphism is selected from one of the genomic mutations shown in FIG. 12.

Suitable tests for assessing the presence of a polymorphism would be well known to the skilled person, and include but are not limited to, Isozyme Electrophoresis, Restriction Fragment Length Polymorphisms (RFLPs), Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length polymorphisms (AFLPs), Simple Sequence Repeats (SSRs-which are also referred to as Microsatellites), and Single Nucleotide Polymorphisms (SNPs). In one embodiment, Kompetitive Allele Specific PCR (KASP) genotyping is used.

In one embodiment, the method comprises
a) obtaining a nucleic acid sample from a plant and
b) carrying out nucleic acid amplification of one or more AAP, preferably AAP8 alleles using one or more primer pairs.

In a further embodiment, the method may further comprise introgressing the chromosomal region comprising an AAP polymorphism into a second plant or plant germplasm to produce an introgressed plant or plant germplasm. Preferably, said second plant will display an increase in seed yield compared to a control or wild-type plant that does not carry the polymorphism.

In a further aspect of the invention there is provided a method for increasing seed yield, the method comprising
a. screening a population of plants for at least one plant with at least one AAP polymorphism as described herein;
b. further modulating the activity of an AAP protein, as described herein, in said plant by introducing and expressing a nucleic acid construct comprising a nucleic acid encoding an AAP polypeptide as described herein, or introducing at least one mutation into the nucleic acid sequence encoding an AAP as described herein.

While the foregoing disclosure provides a general description of the subject matter encompassed within the scope of the present invention, including methods, as well as the best mode thereof, of making and using this invention, the following examples are provided to further enable those skilled in the art to practice this invention and to provide a complete written description thereof. However, those skilled in the art will appreciate that the specifics of these examples should not be read as limiting on the invention, the scope of which should be apprehended from the claims and equivalents thereof appended to this disclosure. Various further aspects and embodiments of the present invention will be apparent to those skilled in the art in view of the present disclosure.

“and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

Unless context dictates otherwise, the descriptions and definitions of the features set out above are not limited to any particular aspect or embodiment of the invention and apply equally to all aspects and embodiments which are described.

The foregoing application, and all documents and sequence accession numbers cited therein or during their prosecution (“appln cited documents”) and all documents cited or referenced in the appln cited documents, and all documents cited or referenced herein (“herein cited documents”), and all documents cited or referenced in herein cited documents, together with any manufacturer's instructions, descriptions, product specifications, and product sheets for any products mentioned herein or in any document incorporated by reference herein, are hereby incorporated herein by reference, and may be employed in the practice of the invention. More specifically, all referenced documents are incorporated by reference to the same extent as if each individual document was specifically and individually indicated to be incorporated by reference.

The invention is now described in the following non-limiting example.

### Example

To understand natural allelic variation at seed size loci, we sought to identify the QTL genes for seed size in Arabidopsis. Cvi (Cape Verde Islands) and Ler (Landsburg erecta) are two Arabidopsis accessions. Cvi seeds were obviously larger and heavier than Ler seeds (FIG. 7) (Alonso-Blanco et al., 1999). By using one recombinant inbred line population from Ler an Cvi, a QTL locus for seed size was previously mapped into the top region of Chromosome I (Alonso-Blanco et al., 1999). To identify the gene corresponding to this QTL for seed size, we obtained the chromosome segment substitution lines (CSSL) that introgressed genomic regions from Cvi accession to the Ler genetic background, which covered this QTL region (Keurentjes et al., 2007). The line CSSL-LCN1-3-3 showed larger and heavier seeds than Ler (FIG. 7), suggesting that this line contained a genomic region from Cvi, which contributes to large and heavy seed phenotypes. To confirm this, we backcrossed the line CSSL-LCN1-3-3 with Ler and generated an F₂ population. Using this F₂ population, we mapped a major QTL locus for grain size and weight on Chromosome I (SSW1) (FIGS. 3A and 3B). We further backcrossed the line CSSL-LCN1-3-3 with Ler for five times and generated a near-isogenic line NIL-SSW1^{Cvi} in the Ler background.

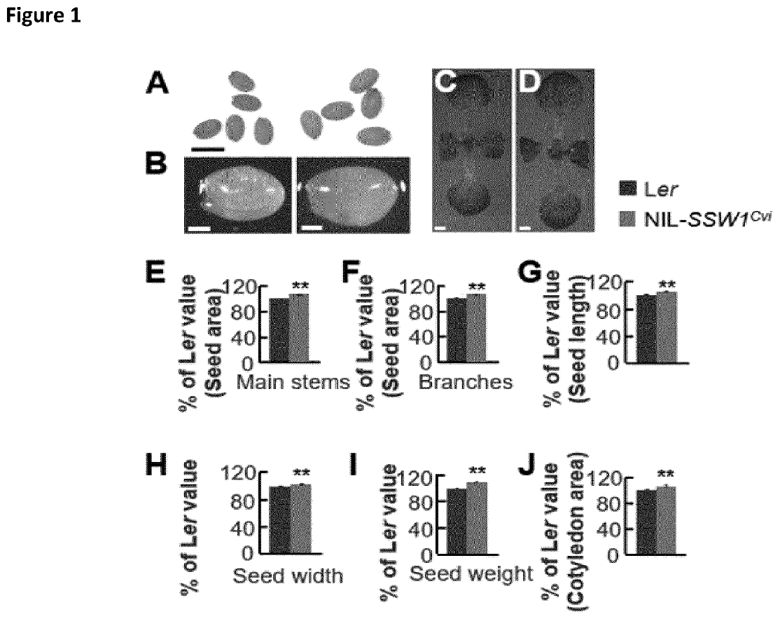

We next investigated grain size and weight of Ler and NIL-SSW1^{Cvi}. As shown in FIG. 1, NIL-SSW1^{Cvi} seeds were significantly larger and heavier than Ler seeds. Consistent with this, the NIL-SSW1^{Cvi} embryos were slightly big compared with Ler embryos (FIG. 1B). The changes in seed size often influence the size of seedlings. Supporting this, the 10-d-old NIL-SSW1^{Cvi} cotyledons were bigger than Ler cotyledons (FIGS. 1C and 1D). By contrast, plant morphology of NIL-SSW1^{Cvi} was similar to that of Ler. The sizes of NIL-SSW1^{Cvi} leaves and floral organs were comparable with that of Ler. These results indicate that SSW1 regulates seed size and weight in Arabidopsis.

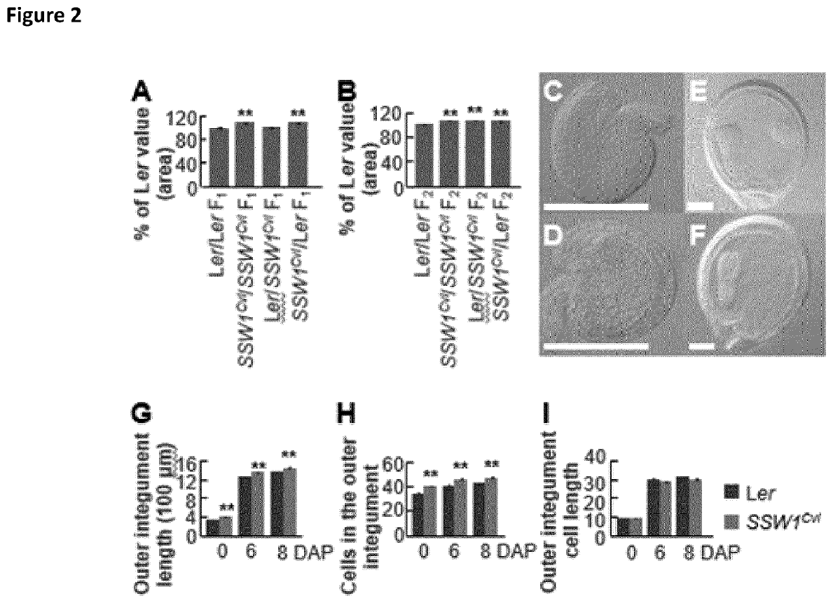

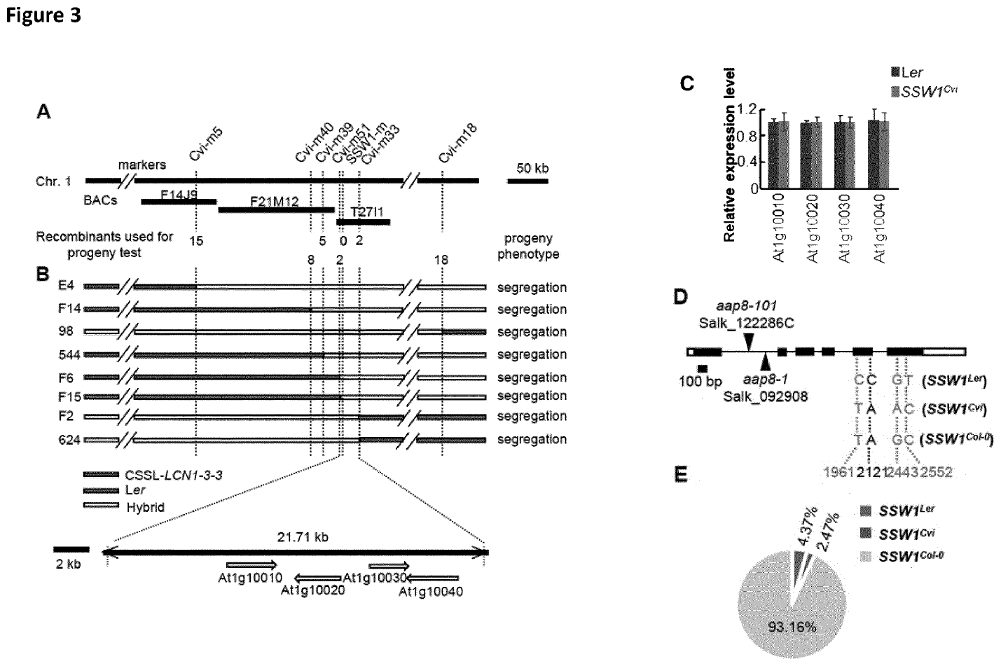

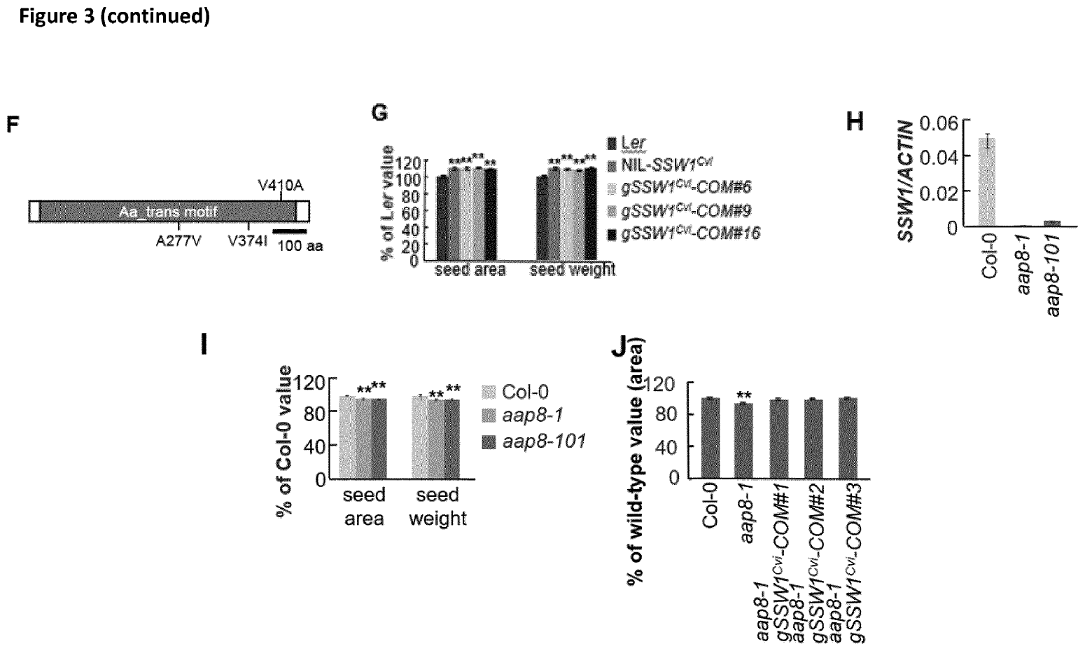

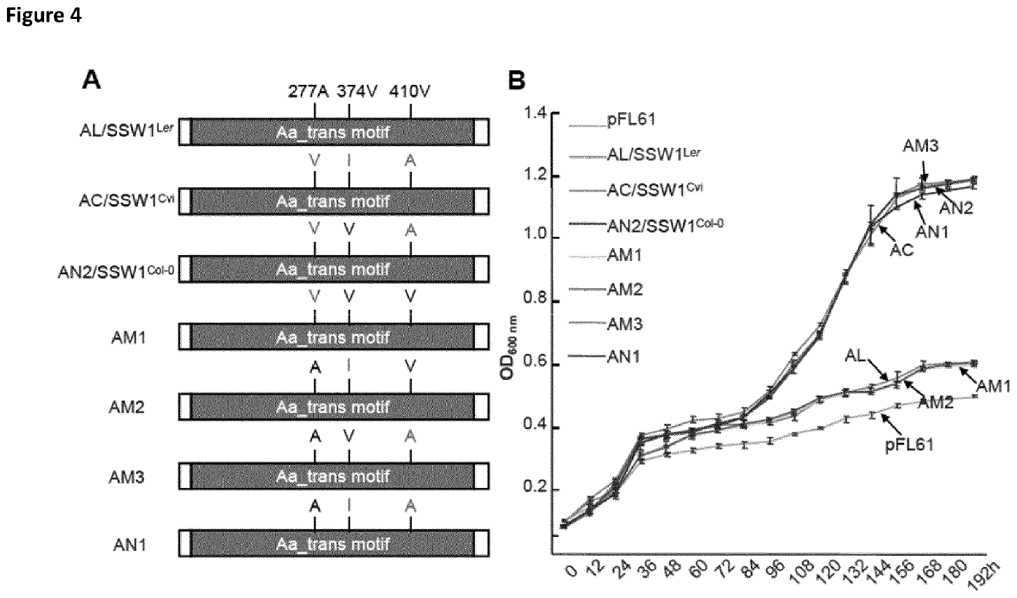

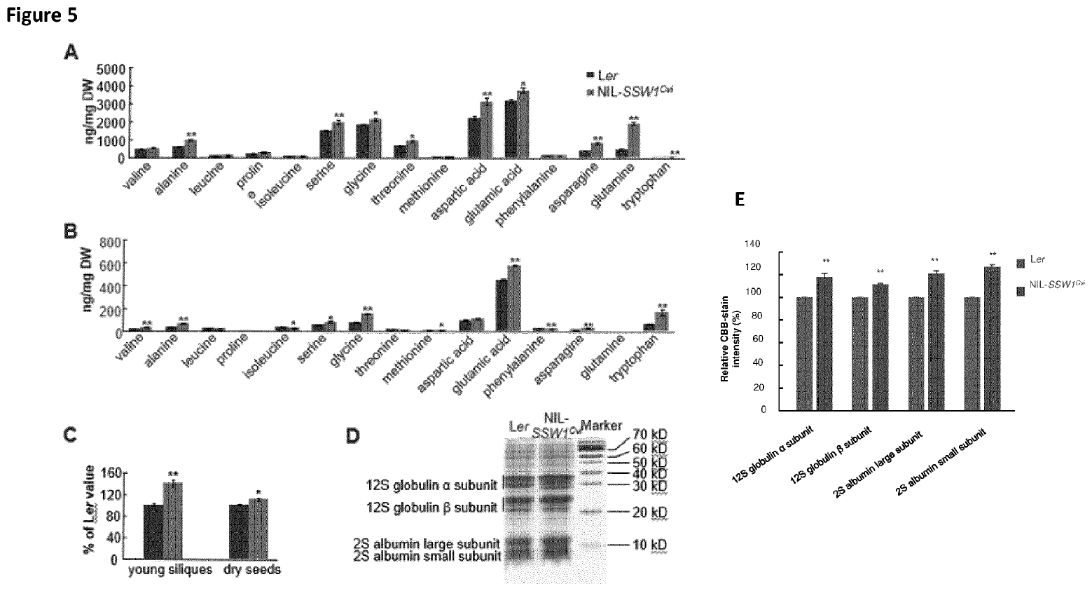

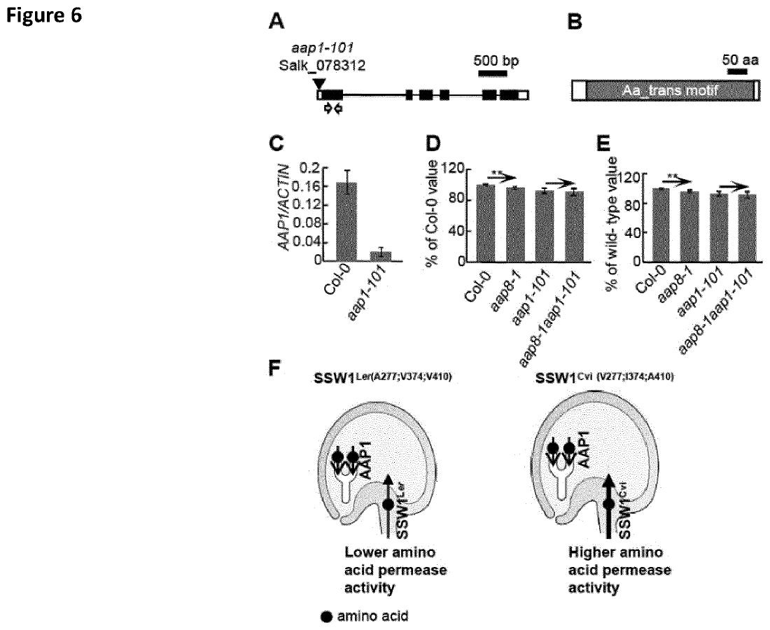

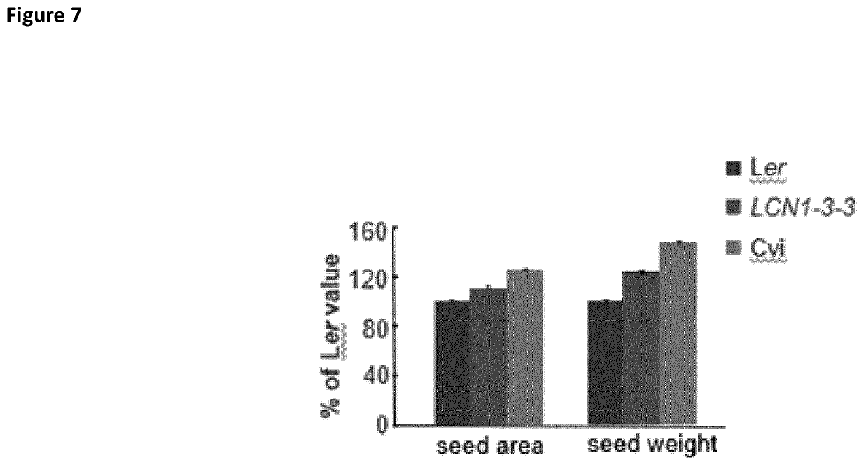

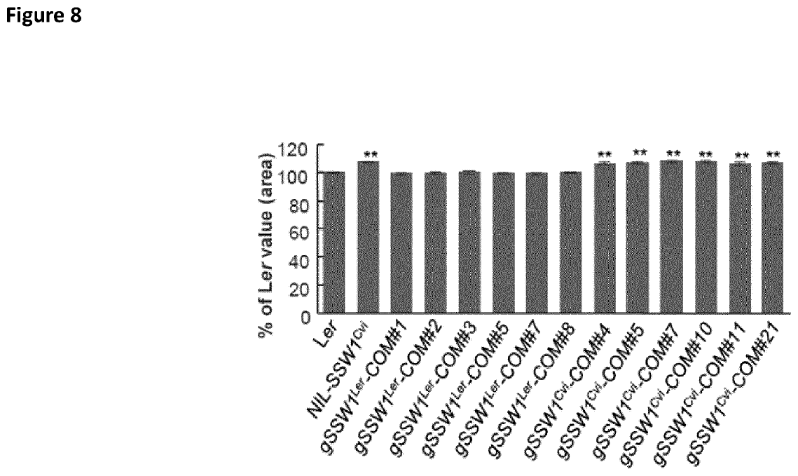

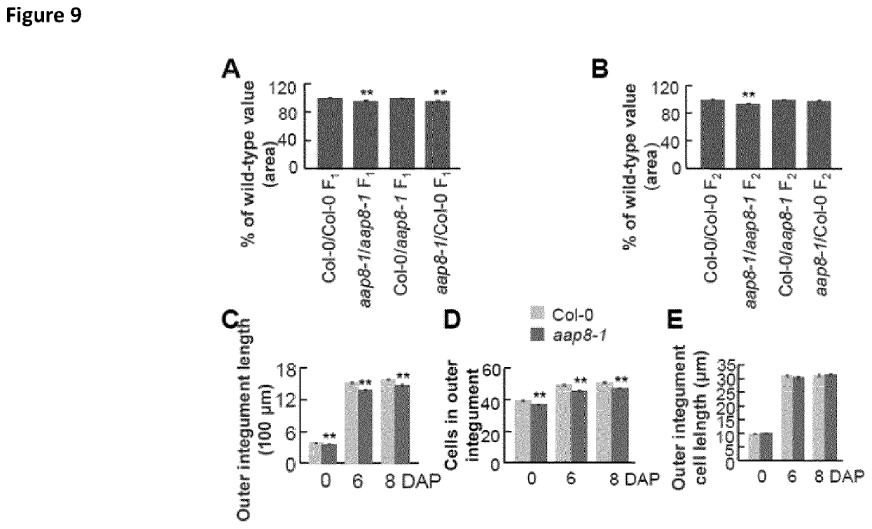

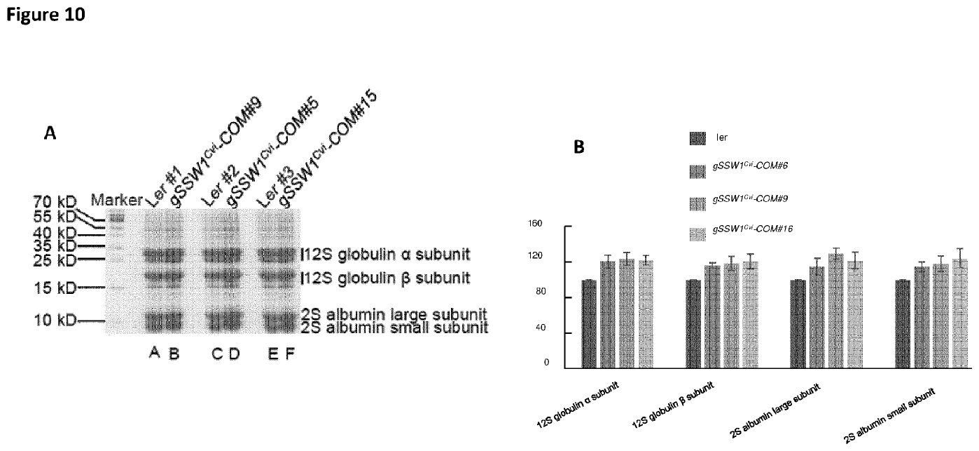

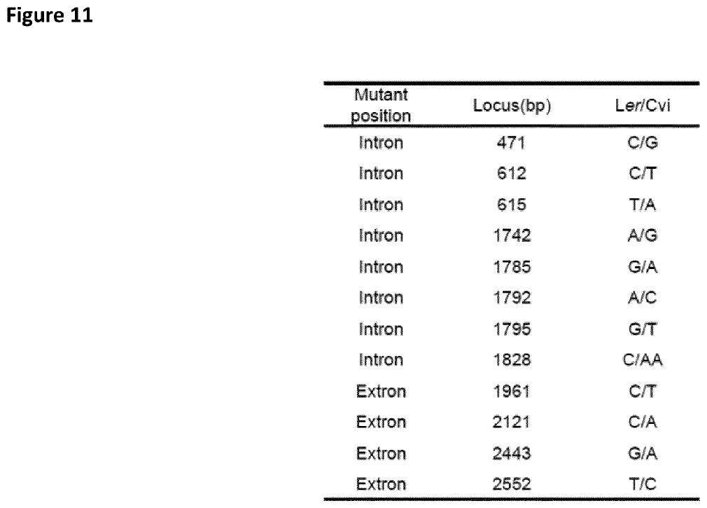

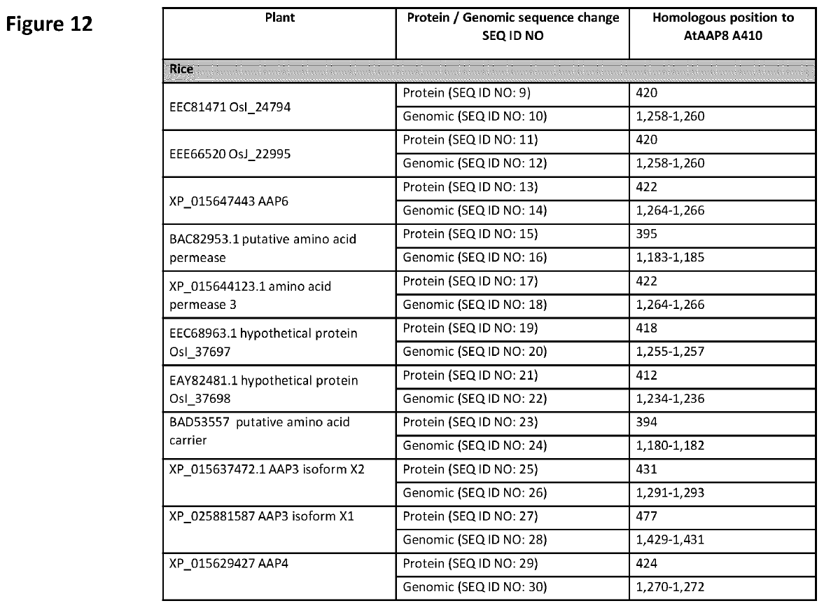

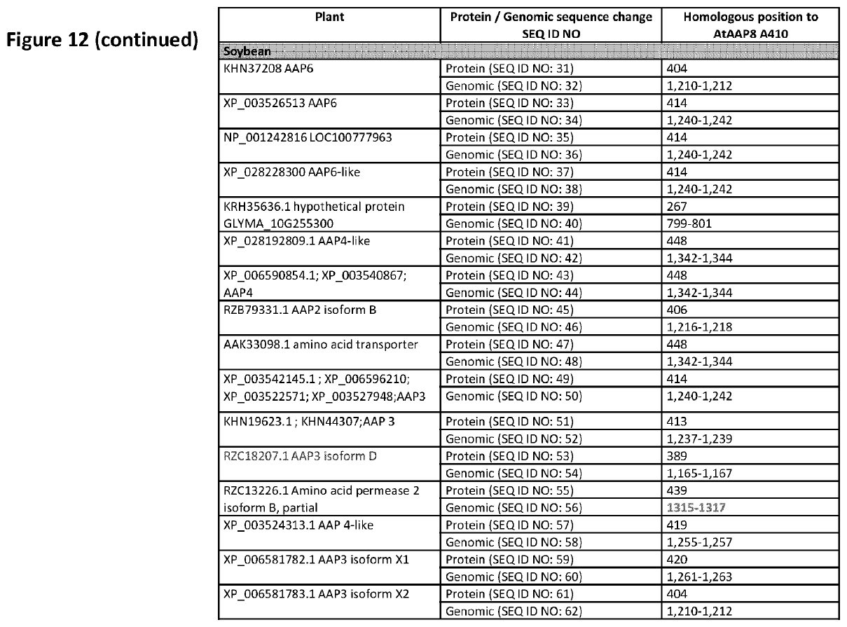

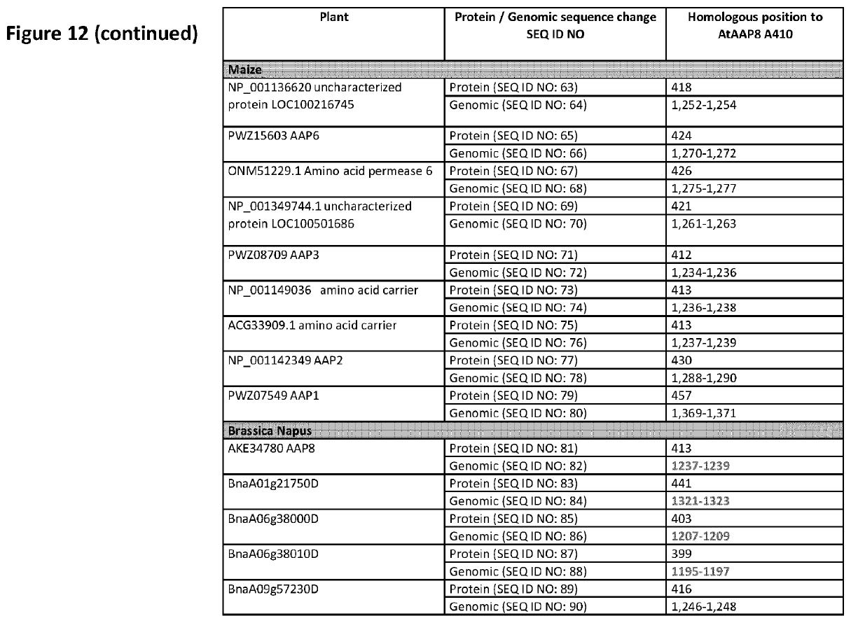

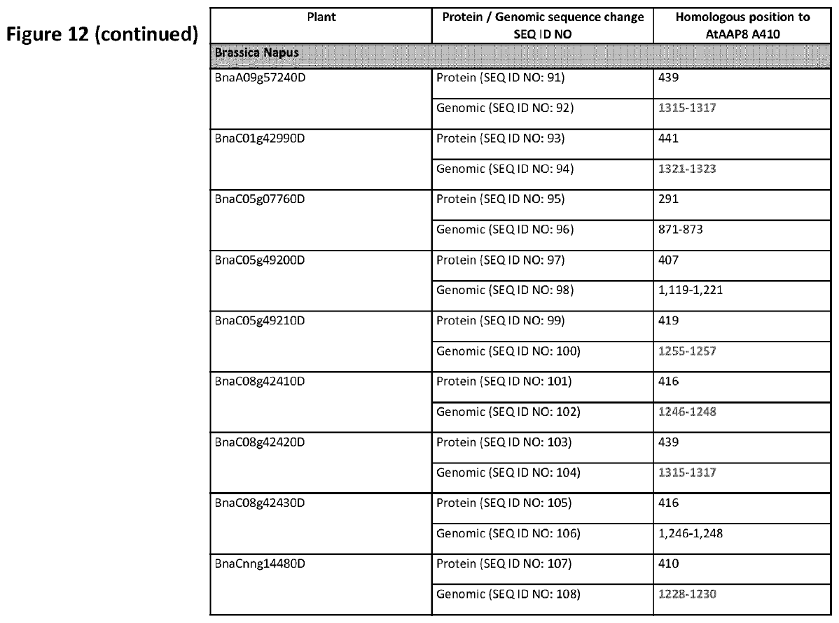

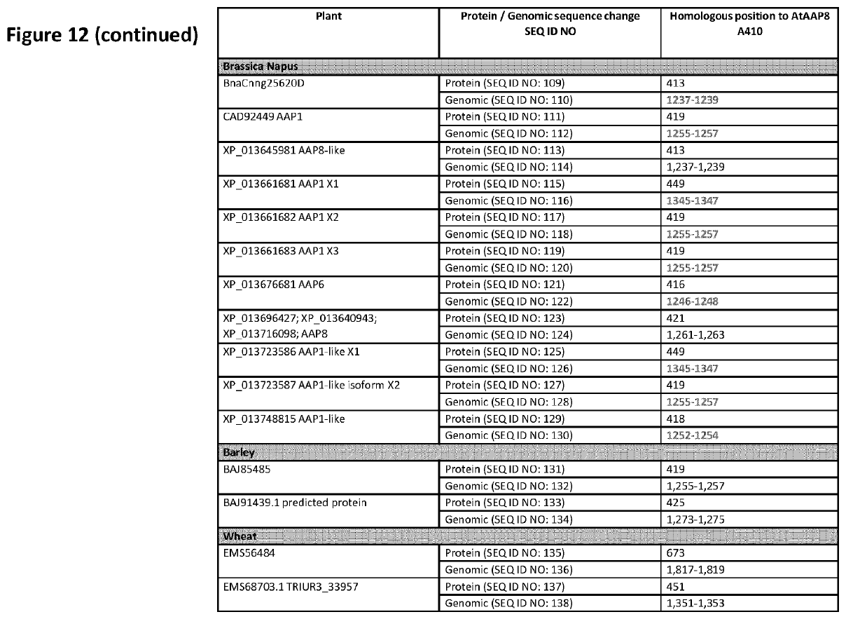

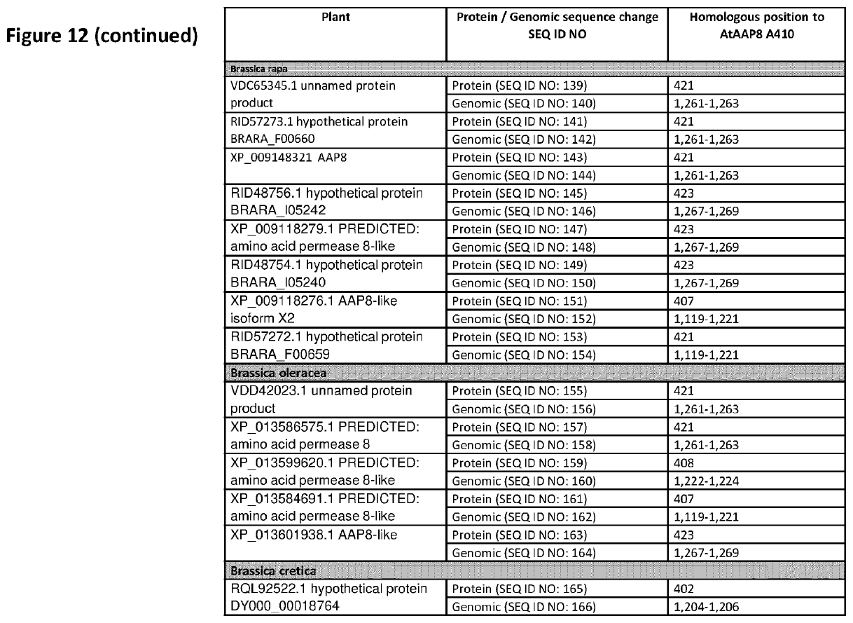

The maternal and/or zygotic tissues have been known to determine the size of a seed (Li and Li, 2016), we therefore asked whether SSW1 acts maternally or zygotically. The reciprocal cross experiments between Ler and NIL-SSW1^{Cvi} were conducted. The size of seeds from NIL-SSW1^{Cvi} plants pollinated with Ler pollen or NIL-SSW1^{Cvi} pollen was significantly larger than that from the self-pollinated Ler plants (FIG. 2A). By contrast, Ler plants pollinated with NIL-SSW1^{Cvi} pollen produced similar-sized seeds to Ler plants pollinated with their own pollen. These results of four crosses show that SSW1 maternally affects seed growth. We further examined the size of Ler/Ler F₂, Ler/NIL-SSW1^{Cvi} F₂, NIL-SSW1^{Cvi}/Ler F₂ and NIL-SSW1^{Cvi}/NIL-SSW1^{Cvi} F₂ seeds. Ler/NIL-SSW1^{Cvi} F₂, NIL-SSW1^{Cvi}/Ler F₂ and NIL-SSW1^{Cvi}/NIL-SSW1^{Cvi} F₂ seeds were significantly larger than Ler/Ler F₂ seeds (FIG. 2B). Thus, these findings reveal that SSW1 controls seed size through maternal tissues. These data also indicate that the SSW1^{Cvi} allele is a dominant allele, while the SSW1^{Ler} allele is a recessive allele.

The integuments surrounding the ovule have been proposed to affect the final size of a seed after fertilization (Adamski et al., 2009; Du et al., 2014; Garcia et al., 2005; Schruff et al., 2006; Xia et al., 2013). Considering that SSW1 affects seed size through maternal tissues, we examined whether SSW1 could control seed size through the maternal integuments. We firstly observed mature ovules before fertilization. As shown in FIGS. 2C and 2D, the NIL-SSW1^{Cvi} mature ovules were obviously larger than Ler ovules. NIL-SSW1^{Cvi} ovules had longer outer integument than Ler ovules (FIG. 2G).

Considering that the growth of the integument is influenced by cell division and cell expansion, we investigated cell number and cell size of the outer integuments in Ler and NIL-SSW1^{Cvi} ovules. The outer integument NIL-SSW1^{Cvi} ovules contained more cells than that of Ler ovules (FIG. 2H). By contrast, outer integument cells in NIL-SSW1^{Cvi} ovules showed similar length to those in Ler ovules (FIG. 2I). These data indicated that SSW1 influences cell proliferation in the integuments of ovules. We further investigated the effect of SSW1 on cell proliferation and cell expansion in the integuments of developing seeds. At 6 days after pollination (6 DAP), the outer integument cells in Ler and NIL-SSW1^{Cvi} seeds absolutely stop division (FIG. 2H). The outer integument in NIL-SSW1^{Cvi} seeds contained more cells than that in Ler seeds (FIG. 2H). By contrast, the length of the outer integument cells in NIL-SSW1^{Cvi} seeds was comparable with that in Ler seeds (FIG. 2I). Taken together, these data demonstrate that SSW1 affects cell proliferation in the maternal integuments of ovules and developing seeds.

To identify the QTL gene for seed size and weight (SSW1), we generated large F₂ population from a cross between the original line CSSL-LCN1-3-3 and Ler. This QTL locus was mapped into the short arm of the chromosome 1 between markers Cvi-m5 and Cvi-m18. We genotyped 10048 F₂ plants using markers Cvi-m5 and Cvi-m18 and identified 867 recombinants. To identify the gene underlying the SSW1 locus, we developed another four markers (Cvi-m40, Cvi-m39, Cvi-m51 and Cvi-m33) in mapping region. We then selected 33 plants with recombinations between these six markers to perform progeny test. Based on progeny test results, we narrowed the candidate gene region containing the SSW1 locus to 21.71 kb between markers Cvi-m51 and Cvi-m33, which contains four genes (At1g10010, At1g10020, At1g10030 and At1g10040) (FIGS. 3A and 3B). Considering that natural mutations could happen in the promoter region, we firstly examined expression levels of these four genes in Ler and NIL-SSW1^{Cvi}. As shown in FIG. 3C, expression levels of these four genes in NIL-SSW1^{Cvi} were comparable with those in Ler, suggesting that natural allelic variation in SSW1 might not affect its expression level. We then sequenced these four genes in Ler, Cvi and NIL-SSW1^{Cvi} Sequence comparison revealed that the predicted amino acid sequences encoded by At1g10020, At1g10030 and At1g10040 in NIL-SSW1^{Cvi} are exactly the same as those in Ler, suggesting that it is unlikely that At1g10020, At1g10030 and At1g10040 are responsible for seed size variation. By contrast, the region of the At1g10010 gene in NIL-SSW1^{Cvi} and Cvi contains 12 single nucleotide polymorphisms compared with that in Ler, including 8 single nucleotide polymorphisms in introns and 4 single nucleotide polymorphisms in exons (FIG. 3D and FIG. 11). Four single nucleotide polymorphisms in exons contain one nucleotide change (C2204A) in the exon 5 that is a synonymous mutation, one nucleotide substitution (C2044T) in the exon 5 that led to an amino acid change from Ala to Val, one nucleotide substitution (G2526A) in the exon 6 that caused an amino acid change from Val to Ile, and one nucleotide substitution (T2635C) in the exon 6 that caused an amino acid change from Val to Ala (FIGS. 3D and 3F). We further developed the marker SSW1-m according to the mutation C1961T in the At1g10010 gene, which was co-segregated with the seed size phenotype (FIG. 3A). Therefore, these results suggest that At1g10010 is a candidate gene for SSW1.

To testify whether natural variation in the At1g10010 gene causes large seeds in Cvi, we conducted a genomic complementation test. Our reciprocal crosses revealed that the Cvi allele is a dominant allele and the Ler allele is a recessive allele (FIGS. 2A and 2B). We therefore introduced a genomic fragment from Cvi that includes 2,631-bp flanking sequence of 5′ UTR, the At1g10010 gene and 671-bp flanking sequence of 3′UTR (gSSW1^{Cvi}-COM) into Ler. Transgenic plants (gSSW1^{Cvi}-COM) produced large and heavy seeds, like those observed in NIL-SSW1^{Cvi} (FIG. 3G and FIG. 8), indicating that At1g10010 is the SSW1 gene. We also introduced the SSW1 genomic fragment from Ler (gSSW1^{Ler}-COM) into Ler. As shown in FIG. 8, the size of gSSW1^{Ler}-COM seeds was similar to that of Ler, indicating that there was no dosage effect in transgenic plants (FIG. 8). These results further support that At1g10010 is the SSW1 gene.

As three nucleotide polymorphisms resulted in amino acid changes between Ler and Cvi (FIG. 3F), we analyzed the sequences of the SSW1 gene in Arabidopsis accessions from 1001 genome project (Genomes Consortium. Electronic address and Genomes, 2016). According to these three polymorphisms, these Arabidopsis accessions contained three types of natural allelic variation in the SSW1 gene, including SSW1^{Cvi}, SSW1^{Ler} and SSW1^{Col-0} types. Most Arabidopsis accessions (93.16%) are the SSW1^{Col-0} type, 4.37% Arabidopsis accessions possess the SSW1^{Ler} type, and 2.47% Arabidopsis accessions belong to the SSW1^{Cvi} type (FIG. 3E). Arabidopsis accessions with the SSW1^{Col-0} type grow in different regions of the world. Interestingly, we found that Arabidopsis accessions with the SSW1^{Ler} type are predominantly distributed in Sweden and Germany, while accessions with the SSW1^{Cvi} type mainly grow in the south of Russia and Spain.

SSW1 encodes the amino acid permease 8 (AAP8) containing an amino acid transporter motif (FIG. 3F). Homologs of AAP8 were found in Arabidopsis and crops. In Arabidopsis, AAP8 belongs to the AAP family that consists of eight members (AAP1-AAP8) (Okumoto, 2002). The AAP family members have been proposed to participate in a variety of physiological processes in plants, such as amino acid transport and xylem-phloem transfer (Tegeder, 2012). Arabidopsis AAP8 mediates amino acid uptake into seeds, but its role in seed size control has not been characterized in detail.

To determine expression of SSW1/AAP8, we conducted quantitative real-time RT-PCR analysis. Relatively higher expression of AAP8 was found in roots, inflorescences, and developing siliques, consistent with a previous study (Okumoto, 2002). AAP8 has been shown to localize in the plasma membrane when SSW1/AAP8-GFP fusion protein was transiently expressed in N. benthamian leaves (Santiago and Tegeder, 2016). However, the subcellular localization of AAP8 in Arabidopsis plants has not been described. We generated 35S:GFP-AAP8 transgenic plants to investigate the subcellular localization of AAP8 in Arabidopsis. GFP signal in 35S:GFP-AAP8 transgenic plants was found at the cell periphery. To examine whether AAP8-GFP was localized in cell walls or the plasma membrane, we used a high concentration of sucrose to induce plasmolysis. GFP signal was detected in the plasma membrane. Thus, these results show that SSW1/AAP8 is a plasma membrane protein in Arabidopsis.

To further investigate the function of loss-of-function of SSW1/AAP8 in seed size, we obtained two mutants (aap8-1/SALK_092908 and app8-101/SALK_122286C) harboring T-DNA insertions in the first intron of the At1g10010 gene, respectively (FIG. 3D). We crossed app8-1 and app8-101 to Col-0 for three times before we investigated their phenotypes. Expression of SSW1/AAP8 was hardly detected in app8-1 and app8-101 mutants (FIG. 3H), suggesting that they are null alleles. We measured seed area and seed weight of app8-1 and app8-101. As shown in FIG. 3I, seed area and seed weight of app8-1 and app8-101 were significantly decreased in comparison to those of Col-0. We introduced the genomic fragment (gSSW1^{Cvi}-COM) from Cvi accession into app8-1 mutant. The gSSW1^{Cvi}-COM fragment complemented the seed size phenotype of the app8-1 mutant, indicating that loss of function of SSW1/AAP8 results in small and light seeds (FIG. 3J).

We then performed the reciprocal cross experiments between Col-0 and app8-1 by hand pollination. As shown in FIG. 9A, app8-1 plants pollinated with Col-0 pollen or app8-1 pollen produced smaller F₁ seeds compared with the F₁ seeds of the self-pollinated Col-0 plants. Col-0/Col-0 F₂, Col-0/app8-1 F₂, and app8-1/Col-0 F₂ seeds were significantly larger than app8-1/app8-1 F₂ seeds (FIG. 9B). Thus, these results further demonstrate that SSW1 is required in maternal tissues to control seed size. We then examined cell number and cell size in the outer integuments and found that SSW1 influences cell proliferation in the maternal integuments of ovules and developing seeds (FIGS. 9D and 9E).

As natural allelic variation in SSW1 contributes to seed size and weight differences between Cvi and Ler, we asked whether natural allelic variation in SSW1 influences the amino acid permease activity of SSW1/AAP8. The yeast mutant strain 22Δ8AA can not use γ-aminobutyric acid, arginine, proline, aspartate, glutamate or citrulline as sole nitrogen sources (Okumoto, 2002). AAP8 has been reported to complement the mutant strain 22Δ8AA (Okumoto, 2002). We therefore expressed the SSW1/AAP8 gene from Cvi (pFL61-SSW1^{Cvi}) and Ler (pFL61-SSW1^{Ler}) in the mutant strain 22Δ8AA, respectively. The 22Δ8AA cells with pFL61-SSW1^{Cvi} formed colonies on plates containing 1 mM and 2 mM ASP as sole nitrogen source after 4 days. By contrast, the 22Δ8AA cells with pFL61-SSW1^{Ler} formed colonies on plates containing 3 mM ASP as sole nitrogen source after 4 days. However, the growth vigor of the 22Δ8AA cells with pFL61-SSW1^{Ler} was obviously lower than that of the 22Δ8AA cells with pFL61-SSW1^{Cvi} on plates supplying 1 mM, 2 mM or 3 mM ASP as sole nitrogen source. These results indicate that the SSW1 from Cvi (SSW1^{Cvi}) has higher amino acid permease activity than that from the Ler allele (SSW1^{Ler}).

To quantify the activity differences between SSW1^{Cvi} and SSW1^{Ler}, we cultured the mutant stain 22Δ8AA harboring pFL61, pFL61-SSW1^{Ler} and pFL61-SSW1^{Cvi} constructs in liquid medium with 1 mM ASP as sole nitrogen source and monitored their growth dynamics by measuring the optical density (OD) at 600 nm every 12 hours. As shown in FIG. 4B, the OD_{600 nm} of the mutant stain 22Δ8AA transformed with pFL61-SSW1^{Cvi} increased drastically after 96 hours, and plateaued after 156 hours. By contrast, the mutant stain 22Δ8AA transformed with pFL61-SSW1^{Ler} showed a slightly faster growth than control (pFL61) (FIG. 4B). These data indicate that SSW1^{Cvi} has higher activity in transporting ASP than SSW1^{Ler}, and SSW1^{Ler} still possesses weak activity in transporting ASP.

As SSW1^{Col-0} has an amino acid change (I374V) compared with SSW1^{Cvi} we investigated the activity of SSW1^{Col-0} in transporting amino acid in yeast cells (FIG. 4A). The mutant stain 22Δ8AA harboring pFL61-SSW1^{Col-0} construct was cultured in liquid medium with 1 mM ASP as sole nitrogen source, and the growth dynamic was detected by measuring the optical density (OD) at 600 nm every 12 hours. The growth dynamic of the mutant stain 22Δ8AA transformed with pFL61-SSW1^{Col-0} was similar to that of the mutant stain 22Δ8AA transformed with pFL61-SSW^{Cvi} (FIG. 4B), indicating that SSW1^{Col-0} has similar amino acid transport activity to SSW1^{Cvi} and possesses higher amino acid transport activity than SSW1^{Ler}. This result also suggests that only one amino acid change (I374 V) does not significantly affect the transport activity.

As there are three amino acid differences between SSW1^{Ler(A277;V374;V410)} and SSW1^{Cvi (V277;I374;A410)}, we asked which amino acid plays a predominant role in determining the activity of SSW1. To test this, we generated AL/SSW1^{Ler(A277;V374;V410)}, AC/SSW1^{Cvi (V277;I374;A410)}, AM1/SSW1^{(V277;V374;V410)}, AM2/SSW1^{(A277;I374;V410)}, AM3/SSW1^{(A277;V374;A410)}, AN1/SSW1^{(A277;V374;A410)}, and AN2/SSW1^{Col-0(V277;V374;A410)} constructs and transformed into the yeast mutant strain 22Δ8AA (FIG. 4A). As shown in FIG. 4B, AN2/SSW1^{Col-0}, AM3/SSW1^{(A277;V374;A410)} and AN1/SSW1^{(A277,I374;A410)} showed similar transport efficiency to SSW1^{Cvi} while the activity of AM2/SSW1^{(A277;I374;V410)} and AM1/SSW1^{(V277;V374;V410)} were comparable with that of SSW1^{Ler}. Thus, these results indicate that the change in the amino acid V410A is mainly responsible for the activity differences between SSW1^{Cvi} and SSW1^{Ler}.

As SSW1 encodes an amino acid permease that has been proposed to transport amino acids to developing seeds (Schmidt et al., 2007), we analyzed the content of free amino acids in young siliques and mature seeds of NIL-SSW1^{Cvi} and Ler by Gas Chromatography-Mass Spectrometer (GC-MS). In young siliques, the contents of some free amino acids such as alanine, serine, aspartic acid, asparagine, and glutamic acid were significantly increased in NIL-SSW1^{Cvi}, while the contents of some amino acids remain the same as Ler (FIG. 5A). In mature seeds, the contents of several amino acids (e.g. valine, alanine, serine, glycine, glutamic acid and tryptophan) in NIL-SSW1^{Cvi} were significantly increased compared with that in Ler (FIG. 5B). Total amino acid contents in NIL-SSW1^{Cvi} siliques and seeds were increased compared with those in Ler siliques and seeds (FIG. 5C). These results indicate that the SSW1^{Cvi} natural allele increases amino acid contents.

We also assayed the content of free amino acids in young siliques and mature seeds of Col-0 and aap8-1. In young siliques, the contents of some free amino acids such as proline, glycine, aspartic acid, glutamic acid, asparagine and glutamine were significantly decreased in aap8-1, while the contents of some amino acids were similar to those in Col-0. In mature seeds, the contents of several amino acids (e.g. valine, leucine, isoleucine, serine, glycine, threonine, aspartic acid, glutamic acid, phenylalanine and tryptophan) in aap8-1 were significantly decreased compared with that in Col-0. In addition, total amino acid contents in the siliques and seeds of app8-1 were lower than those in wild-type (Col-0) siliques and seeds.

We then analyzed the content of soluble proteins in Ler and NIL-SSW1^{Cvi} dry seeds by SDS-PAGE. The contents of 12S globulin α subunit, 12S globulin β subunit, 2S albumin large subunit and 2S albumin small subunit in NIL-SSW1^{Cvi} seeds were obviously increased compared with those in Ler seeds (FIG. 5D). These results indicate that the SSW1^{Cvi} natural allele seeds contain more storage proteins than Ler. We then measured the content of soluble proteins in dry seeds of Ler and three gSSW1^{Cvi}-Com transgenic lines. The contents of 12S globulin α subunit, 12S globulin β subunit, 2S albumin large subunit and 2S albumin small subunit in seeds of gSSW1^{Cvi}-Com transgenic lines were obviously increased compared with those in Ler seeds (FIG. 10).

As AAP8/SSW1 exhibits the highest similarity to Arabidopsis AAP1, which has been reported influencing seed weight (Sanders, 2009), we asked whether there are any genetic relationship between aap8-1 and aap1 in seed size control. To test this, we obtained aap1-101 (Salk_078312) (FIGS. 6A to 6C). The aap1-101 seeds were significantly smaller than Col-0 seeds (FIGS. 6D and 6E), consistent with the result that aap1 seeds were lighter than wild-type seeds (Sanders, 2009). We crossed aap8-1 with app1-101 and generated aap8-1 app1-101 double mutant. The seed size and weight of the aap8-1 aap1-101 double mutants were not significantly decreased compared with those of aap8-1 (FIGS. 6D and 6E), suggesting that AAP8 may act, at least in part, genetically with AAP1 to affect seed size and weight.

Discussion

Seed size is an important yield trait and is controlled by quantitative trait loci. Several QTLs for seed size have been mapped in Arabidopsis, but the genes corresponding to these QTLs have not been cloned yet. In this study, we cloned the first QTL gene for seed size and weight (SSW1) in Arabidopsis and find that natural allelic variation in SSW1 contributes to seed size, weight and quality. SSW1 encodes an amino acid permease (AAP8) that transports amino acids into seeds. Natural allelic variation in SSW1 affects the amino acid permease activity, thereby influencing the contents of free amino acids and storage proteins in seeds. Therefore, these results reveal the genetic and molecular basis for natural variation in seed size, weight and quality control, suggesting that it is an important target for improving both seed size and quality in crops.

Several QTL loci for seed size were mapped in different chromosomes of Arabidopsis using the recombinant inbred line population from Ler and Cvi (Alonso-Blanco et al., 1999), but the QTL genes for seed size have not been identified in Arabidopsis. In this study, we fine-mapped a major QTL locus for grain size and weight (SSW1) and cloned the SSW1 gene in Arabidopsis. NIL-SSW1^{Cvi} produced larger and heavier grains than Ler. By contrast, NIL-SSW1^{Cvi} exhibited similar plant architecture, flower size and leaf size to Ler, suggesting that SSW1 mainly controls seed size and weight in Arabidopsis. Cellular observations show that SSW1 controls seed size by promoting cell proliferation during ovule and seed development. SSW1 encodes the amino acid permease AAP8. In Arabidopsis, AAP8 belongs to the AAP family that consists of eight members (AAP1-AAP8) (Okumoto, 2002). The AAP family members have been proposed to participate in a variety of physiological processes in plants, such as amino acid transport and xylem-phloem transfer (Tegeder, 2012). OsAAP6 has been proved to enhance grain protein content and nutritional quality greatly in rice (Peng et al., 2014). In Arabidopsis, AAP8 mediates amino acid uptake into developing seeds, but its role in seed size control has not been characterized in detail. Here we demonstrate natural allelic variations in AAP8 contribute to grain size and weight. AAP8 acts as a positive factor of seed size and weight control in Arabidopsis. Interestingly, a previously study proposed that loss of function of AAP8 resulted in significant seed abortion (Schmidt et al., 2007) and heavy seeds (Santiago and Tegeder, 2016). It is possible that seed abortion might cause heavy seeds.

In this study, we found that the NIL-SSW^{Cvi} had a similar ratio of seed abortion to Ler. Similarly, aap8-1 and aap8-101 mutations did not affect seed abortion compared with the wild type Col-0 under our growth conditions. We also have sufficient evidence to demonstrate that SSW1/AAP8 positively influences seed size and weight. Expression of SSW1/AAP8 complemented the small seed phenotype of aap8-1 (FIG. 3J). In addition, transformation of the genomic sequence of SSW1^{Cvi} into Ler background resulted in large and heavy seeds (FIG. 3G and FIG. 8). The natural allele SSW1^{Cvi} enhanced the large seed phenotype of da1-1^{Ler} and bb-1, which have been known to form large seeds (Li et al., 2008b; Xia et al., 2013), suggesting that SSW1/AAP8 may act independently of DA1 and BB to control seed size and also indicating that the SSW1^{Cvi} allele promotes seed growth in Arabidopsis. Thus, our data demonstrate that SSW1/AAP8 positively influences seed size in Arabidopsis.

Sequence analyses reveal that Arabidopsis accessions possess three main types of natural allelic variation in the SSW1/AAP8 gene, including SSW1^{Cvi}, SSW1^{Ler} and SSW1^{Col-0} types. Most Arabidopsis accessions contain the SSW1^{Col-0} type, 4.37% Arabidopsis accessions are the SSW1^{Ler} type, and 2.47% Arabidopsis accessions belong to the SSW1^{Cvi} type (FIG. 3E). We found that that SSW1^{Cvi} has higher amino acid permease activity than SSW1^{Ler}. SSW1^{Cvi} showed similar amino acid permease activity to SSW1^{Col-0} but higher activity than SSW1^{Ler}, indicating that the natural allele SSW1^{Ler} is a partial loss of function allele. As SSW1^{Col-0} has an amino acid change (I374V) compared with SSW1^{Cvi}, I374V change may not strongly affect the activity of SSW1. There are three amino acid differences between SSW1^{Ler(A277; V374;V410)} and SSW1^{Cvi (V277,I374;A410)} (FIG. 3F). Our results showed that the change in the amino acid V410A are predominantly responsible for the differences of amino acid permease activity between SSW1^{Cvi} and SSW1^{Ler}. Thus, our findings reveal that natural variation in SSW1 leads to changes in amino acid permease activity, there by influencing seed size and weight (FIG. 6F). Higher amino acid permease activity in Cvi accession causes large seeds (FIG. 6F). Interestingly, Arabidopsis accessions with the SSW1^{Col-0} type grow in different parts of the world, accessions with the SSW1^{Ler} type are predominantly distributes in Sweden and Germany, and accessions with the SSW1^{Cvi} type mainly grow in the south of Russia and Spain. It is possible that the locations of SSW1^{Cvi} and SSW1^{Ler} types may reflect the demographic history of Arabidopsis thaliana (Genomes Consortium. Electronic address and Genomes, 2016).

The growth of seeds depends on nitrogen and carbon import from the maternal tissues into developing seeds. Amino acids, the important transport form of nitrogen, are mainly assimilated within plant roots or leaves and then transported to developing fruits and seeds. Arabidopsis AAP8 has been reported to transport amino acids from roots to developing seeds (Schmidt et al., 2007). AAP8 was also crucial for the uptake of amino acids into endosperm (Schmidt et al., 2007). AAP8 is expressed in maternal tissues, such as roots, leaves, flower buds, siliques, funiculi and young seeds (Okumoto, 2002). Thus, it is possible that the delivery of amino acids and carbon from maternal tissues (e.g. roots, leaves, flower buds and siliques) to developing seeds is important for seed size and weight control. Consistent with this, reciprocal cross experiments indicate that SSW1 influences seed size through maternal tissues. Similarly, expression of sucrose transporter (AtSUC2) driven by the phloem protein 2 promoter resulted in large grains in rice (Wang et al., 2015). Arabidopsis AAP1, the closest homolog of AAP8, has been reported to regulate import of amino acids into roots and subsequent translocation into the shoots as well as import of amino acids from the endosperm to the embryo (Lee et al., 2007; Sanders, 2009). Our genetic analyses suggest that AAP8 acts, at least in part, genetically with AAP1 to affect seed size and weight. It is possible that AAP8 and AAP1 might act different steps to transport amino acids to seeds (FIG. 6F). We further showed that the NIL-SSW1^{Cvi} seeds contained more free amino acids and storage proteins than Ler seeds, indicating that AAP8 regulates both seed weight and seed quality (FIGS. 5A to 5D). Thus, our findings reveal the genetic and molecular basis for natural variation of SSW1/AAP8 in seed size, weight and quality control. Our current understanding of natural allelic variation in SSW1/AAP8 suggests that AAP8 and its orthologs in crops (e.g. oilseed rape and soybean) could be used to increase both seed size and seed quality in crops.

Materials and Methods

Plant Materials and Growth Conditions

The near isogenic line CSSL-LCN1-3-3 derived from a cross between two Arabidopsis thaliana ecotypes Ler (Landsberg erecta) and Cvi (Cape Verde Islands). The CSSL-LCN1-3-3 line was backcrossed with Ler for five times to generate the near isogenic line NIL-SSW^{Cvi}. The aap8-1 (SALK_092908), aap8-101 (SALK_122286C) and aap1-101 (SALK 078312) were obtained from the NASC and backcrossed into Col-0 for three times. Arabidopsis plants were grown in greenhouse under long-day conditions at 22° C.

Map-Based Cloning, Constructs and Plant Transformation

The SSW1 gene was mapped using the F₂ population of a cross between CSSL-LCN1-3-3 and Ler. By using this F₂ population, we mapped a major QTL locus for grain size and weight (SSW1). This QTL locus was mapped into the short arm of the chromosome 1 between markers Cvi-m5 and Cvi-m18. To identify the gene underlying the SSW1 locus, we genotyped 10048 F₂ plants with newly-developed markers in the mapping region. We selected 33 recombinants between these markers to perform progeny test. Based on progeny test results, we narrowed the candidate gene region containing the SSW1 locus to about 21.71 kb between markers Cvi-m51 and Cvi-m33, which contains four genes (At1g10010, At1g10020, At1g10030 and At1g10040).

The 2,631-bp flanking sequence of 5′ UTR, the At1g10010 gene and 671-bp flanking sequence of 3′UTR from SSW1^{Cvi} and SSW1^{Ler} were amplified using the primers SSW1-gP-1F and SSW1-g3U-1R. To generate gSSW1^{Cvi}-COM and gSSW1^{Ler}-COM constructs, we ligased PCR product to pCR8/GW/TOPO vector, and then ligased to the pMDC99 binary vector using LR reaction (Invitrogen). We transformed the plasmids gSSW1^{Cvi}-COM and gSSW1^{Ler}-COM into the Ler using Agrobacterium tumefaciens line GV3101, and then selected transformants using MS medium supplied with hygromycin (30 μg/mL). We transformed the plasmid gSSW1^{Cvi}-COM into the aap8-1 using the same way.

The 1425-bp coding region of SSW1/AAP8 gene from Col-0 was amplified using primers SSW1-cS-F and SSW1-cE-R. To construct p35S:GFP-SSW1^{Col-0}, we subcloned PCR product to pCR8/GW/TOPO vector, and then ligased to the pMDC43 binary vector using LR reaction (Invitrogen). We transformed the plasmid p35S:GFP-SSW1^{Col-0} into the Col-0 using Agrobacterium tumefaciens line GV3101, and selected transformants using MS medium supplied with hygromycin (30 μg/mL).

Morphological and Cellular Analysis

Mature dry seeds from 3rd-10th siliques of main stems, cotyledons, leaves and floral organs were harvested to measure their sizes as described previously (Zhang et al., 2015). Mature ovules and developing seeds were photographed using differential interference contrast (DIC) microscope (Leica DM2500) to count cells in the outer integument and measure the length of the outer integument by Image J software.

Subcellular Localization

The Zeiss LSM 710 NLO confocal microscope was used to observe GFP fluorescence signals. Petals were treated with 25 μg/μL propidium iodide and 1 μg/mL fm4-64 to stain cell wall and plasma membrane, and treated with 30% sucrose solution for plasmolysis.

RNA Isolation, RT-PCR and Quantitative Real-Time RT-PCR Analysis

RNAprep pure plant kit (Tiangen) was used to extract total RNA. SuperScript III reverse transcriptase (Invitrogen) was used to reversely transcribe into cDNA. The 7500 Real-Time PCR System (Applied Biosystems) was used to conduct Quantitative real-time RT-PCR (QRT-PCR). An internal control is ACTIN2 mRNA.

Protein and Free Amino Acid Analysis

Extraction of soluble protein was conducted according to Sanders et. al. (Sanders, 2009) with modification. A batch of 100 dry mature seeds were grounded in 200 μL extraction buffer [10% (v/v) glycerol, 100 mM Tris-HCl, 2% (v/v) β-mercaptoethanol and pH 8.0, 0.5% (w/v) SDS]. The resulting 40 μL supernatant after centrifugation in 20,000 g for 10 min was moved to a 1.5 mL microfuge tube and again centrifugated in 20,000 g for 5 min. 4 μL loading buffer [10% (v/v) glycerol, 62.5 mM Tris-HCl, β-mercaptoethanol, 8 M Urea and, 2% (w/v) SDS]. 20 μL supernatant was added into 2 μL bromophenol blue, boiled at 98° C. for 15 min and loaded onto a 15% SDS-PAGE for about 130 min at 100 V after a brief centrifugation.

Free amino acid assays were conducted according to a previously report (Tan et al., 2011). The concentration of free amino acids was calculated by internal standard method, and normalized to the unit dry weight of sample.

Yeast Growth Assay

The coding region sequence of SSW1/AAP8 gene was amplified from SSW1^{Cvi} and Ler cDNA library using primers L-cS-pFL61-infu-F1 and L-cE-pFL61-infu-R2, and then subcloned into yeast expression vector pFL61 to generate the AL and AC plasmids, respectively. The AL and AC constructs and the empty vector were transformed into 22Δ8AA. The transformants were selected on SD/-Ura with Agar media (Clontech Cat. No. 630315, Lot. No. 1504553A). Growth assays were performed on M.am media (Jacobs et al., 1980) lacing uracil with 2.5% (w/v) agar and aspartate at concentrations of 1, 2, 3 mM. Monoclonal transformants were incubated in liquid YPDA media and cultured at 30° C., 200 rpm for about 8-12 h until OD_{600 nm}≈1. After centrifugation precipitates were washed with 0.9% NaCl for three times. We equalized OD_{600 nm} of all samples of yeast cells to about 0.5 with sterilized 0.9% NaCl, and then stroke 10 μL mixture onto plates and culture at 30° C. All experiments were repeated three times with independent colonies.

Site-directed mutagenesis PCR products harboring different nucleotide variations were amplified using primers L-cS-pFL61-infu-F1, L-cE-pFL61-infu-R2 and L-M1-R1, L-M1-F2, L-M2-R1, L-M2-F2, L-M3-R1, L-M3-F1, L-N1-R1, L-N1-F2, L-N2-R1, L-N2-F2, by leading false priming into primers, and then PCR products were subcloned in pFL61 to generate plasmids AM1, AM2, AM3, AN1 and AN2. Plasmids AL, AC, AM1, AM2, AM3, AN1, AN2 and empty vector were transformed into yeast strain 22Δ8AA.

For yeast growth dynamics assays, monoclonal transformants were incubated in liquid YPDA media and cultured at 30° C., 200 rpm for about 8-12 h until OD_{600 nm}≈1. Precipitates after centrifugation were washed with 0.9% NaCl for three times. Yeast cells were added into 5 mL M.am media with 1 mM aspartate (the OD_{600 nm}≈0.1), cultured at 30° C., and used to measure the OD_{600 nm} every 12 hours.

Sequence Listing

Examples of suitable mutation positions (in the wild-type sequence) or mutated nucleotides/amino acids (in the mutated sequences) are highlighted. The invention is not limited to these mutation positions.

SEQ ID NO: 1: AtAAP8^{Ler} (protein)

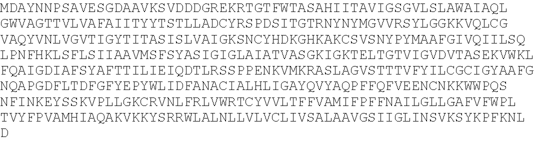

SEQ ID NO: 2 AtAAP8^{Cvi} (protein)

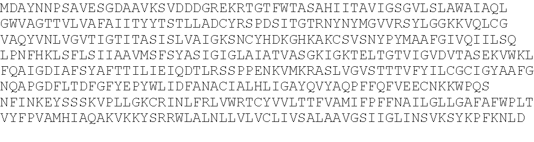

SEQ ID NO: 3: AtAAP8^{Col-0} (protein)

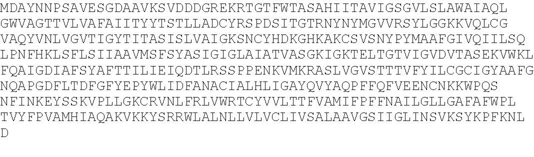

SEQ ID NO: 4: AtAAP8 A410 (protein)

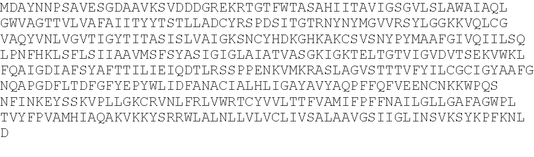

SEQ ID NO: 5: AtAAP8^{Ler} (genomic) (Introns are underlined)
AGGGAGTACTCTAATAAGACGACCTCTGTCAATAACTCTCTTCCCCTCTCTTCTCT
CCTCTGGTTCAGTGGTTCTCTCACAATGATGGACGCATACCACAATCCTTCGGCG
GTGGAGTCGGGTGACGCCGCCGTGAAAAGCGTCGACGACGATGGTCGAGAGAA
GAGAACGGGAACATTTTGGACGGCGAGTGCGCACATAATCACGGCGGTCATAGG
CTCAGGGGTGCTGTCGTTGGCTTGGGCTATAGCACAGCTTGGTTGGGTGGCAGG
AACCACAGTTTTGGTCGCTTTCGCCATCATTACTTACTACACGTCCACCTTGCTCG
CCGACTGTTACCGTTCGCCGGACTCCATCACCGGAACACGCAACTATAATTACAT
GGGCGTCGTCCGATCTTACCTTGGTATGGATTCATATAAACAAATTCATTTTGTGT
CTTTATCAGCATTGTTTTTCACAGATTTTTCAGTTTTCTAGACATTTTTTCTCAGATG
AACAAGGATTTTGTTCATTTGATATCATTTAGATTTTGCCTAACTAGTCTCAATTTAC
GACATGTGTTTTGATTTTCTTCCATTTCTGTCACAATGATGATGGCTGGCGAAAGA
AAAAAAATCTGATCTAAAAATATATATTTAATGCTAAGTTGGAATTTGTAAATCTACA
GTATAATTGGCTCATTTCAACAATTTCTTTCCATGTAAATTTGTTGAAGAACATTATT
GTTGTTGAACAATGAAAGAAAAAAATATGGTTGTTAGAAAAAAATGATTTACGATTT
TGCCAAGTGTGCATGCTCTTTCATGGGAAGATATGAATTAATTATCAAAATCTATAT
AAAAAAAAGGAAGATAATCTTCATTCTTTTCATAACTTAGTTAATAAATTAAATTGATT
AGGATTGGTAACATAGTCAATTCAATTTATCCCGTTAAAGAATGTTATAAATTCGAT
TGTTGACCCCTCGTTGAAAATTTGGAATTATGCGGGATGTTTAGAAACTTTGCCAT
AAGACCAAAAGATTGGTAGTATTTGATAGTAGTACAAGAGTAATCATTTTTCTTCTT
TAATAACATAAAACGCAGGTGGTAAAAAGGTTCAGCTATGTGGAGTGGCACAGTA
CGTGAATCTCGTAGGGGTCACTATTGGTTACACCATCACTGCCTCCATAAGCTTAG
TGTAAGTCAAAGATTCTGATTTATTTCGATTATTTTGTTATGGTTATACTAACATGTT
GTTCTGAATAAAAATTACTAATAATTGTTTGATTGGTGTTTTTGTACGTCTTCGTTAG
AGCGATTGGGAAATCAAATTGTTATCATGACAAGGGACATAAAGCGAAATGTTCTG
TATCGAATTATCCATACATGGCGGCATTTGGGATCGTCCAGATCATTCTGAGCCAG
CTTCCTAACTTCCACAAGCTCTCTTTCCTATCCATCATCGCCGCGGTTATGTCCTT
CTCTTATGCGTCTATCGGAATAGGCCTAGCCATCGCTACTGTAGCAAGTACATTCC
CCTTCTTTATCTTAAAACATAGTGGTTTATATGGATGATTCTTCAAAGTTGACACTA
ACCGTGAAAATGGTATACAATATATATGAAAGGTGGGAAGATTGGTAAGACAGAAT
TGACAGGGACAGTGATAGGTGTGGACGTAACTGCGTCTGAAAAAGTTTGGAAATT
GTTTCAAGCGATTGGAGACATTGCCTTTTCATACGCTTTTACCACTATTCTCATCGA
GATTCAGGCATGTACTACTGATTCCTACTATCTTCCGTTTACTATTGTTTTCATTTG
CTTGTTATTATTAATTTCGCCAAAAAGAGGTAAAATAAGAATACCTTGAAGATAAGA
TGTTATTATTGATTAGAAAGGTAGGAAAAAATATAGATGGATGGATGATGGATCAA
ATAGTTTCATATTTTAGATATGTGAAGCTCTAAAGATAGTGACGCTCTAGTAGTATG
TCTTGTTTATTTTGCAGGACACATTGAGATCAAGCCCACCAGAGAACAAAGTGATG
AAACGAGCAAGTCTTGCCGAGTCTCAACCACAACTGTTTTCTACATCTTGTGTGG
TTGCATCGGATATGCTGCGTTCGGCAACCAAGCCCCTGGTGACTTCCTTACCGAT
TTTGGTTTTTACGAACCTTATTGGCTCATCGACTTTGCCAATGCTTGCATTGCTCTC
CATCTAATCGGTGCCTATCAGGTATAACTCACAAACAAAAGAATAGGATAAGTGTG
TAACATACATTTACCGTGTTCAAGTTCATTAAAAGTCTCATTATTGTGTTAGAATTTT
TAGCTTTAACAATTCAGAAGATTGTAGAAATGGAGTTATTACTAAATATTGTTTCTA
AAAAATGCTCTTTTTTTTTTTTTATCCCTGTATTATTCGCAGGTGTATGCGCAGCCG
TTTTTCCAGTTTGTTGAGGAAAACTGCAACAAAAAATGGCCTCAAAGCAATTTCAT
CAACAAAGAATACTCGTCAAAGGTTCCTTTGCTTGGAAAATGTCGTGTCAACCTCT
TCAGACTGGTTTGGAGGACATGCTATGTTGTTTTGACAACATTTGTAGCAATGATA
TTCCCCTTCTTCAATGCGATCTTGGGTTTGCTAGGGGCATTCGTGTTCTGGCCACT
CACAGTTTATTTTCCGGTGGCAATGCACATTGCGCAGGCTAAAGTCAAGAAGTATT
CTCGTAGATGGTTGGCCTTGAACCTCCTCGTATTGGTTTGCTTGATCGTCTCGGC
CCTTGCCGCCGTAGGATCCATCATTGGCTTAATTAATAGTGTCAAGTCATACAAGC
CCTTCAAGAATTTAGACTAGTGTGACTTATAATCTATGTTTGCCAAAAAAAAACCTT
GTGATCCATATGAAATTTATTTCATGCTAAATATTTAGTACTTAATGTTTCTCCAAAT
AATGTGACGTTCTGTTTTCAGCTATGTTAAAAAACAAAATGCTAACTTGTGTATTAG
TACTAAAATTTATGAAAATGTATTAGTTATTGATTTATTTTTAGGACTACAATTATTG
AATCAACATTGGATGTTTGAGTCCCATGAGATATGGATTTCAGCTTTTTTCAAATTC
GTGTGGTTGTGTCAATTTCGAGTTATTATTATTTATTTTGCTTAATGGATTGTCGG
GGAAATCTTGAAAACAGACACTCACAGATTGTGTAATTTATTTGGTTTGGTGTGTC
CTACATAAGTTGCTATCACATCTTATGTATTGGAGGAGTTGGGCAATAGAGGATCA
AGGCAAGTTTGGTTTTCTATTAACGTTTCTACTCTGCATTTGCTTACAAAGTCATTT
TCAAGGTTTTGTGGTCGTATGTCACTTGATGG
SEQ ID NO: 6: AtAAP8^{Cvi} (genomic) (Introns are underlined)
AGGGAGTACTCTAATAAGACGACCTCTGTCAATAACTCTCTTCCCCTCTCTTCTCT
CCTCTGGTTCAGTGGTTCTCTCACAATGATGGACGCATACCCAATCCTTCGGCGG
TGGAGTCGGGTGACGCCGCCGTGAAAAGCGTCGACGACGATGGTCGAGAGAAG
AGAACGGGAACATTTTGGACGGCGAGTGCGCACATAATCACGGCGGTCATAGGC
TCAGGGGTGCTGTCGTTGGCTTGGGCTATAGCACAGCTTGGTTGGGTGGCAGGA
ACCACAGTTTTGGTCGCTTTCGCCATCATTACTTACTACACGTCCACCTTGCTCGC
CGACTGTTACCGTTCGCCGGACTCCATCACCGGAACACGCAACTATAATTACATG
GGCGTCGTCCGATCTTACCTTGGTATGGATTCATATAAACAAATTCATTTTGTGTCT
TTATCAGCATTGTTTTTCACAGATTTTTCAGTTTTCTAGACATTTTTTCTCAGATGAA
CAAGGATTTTGTTCATTTGATATCATTTAGATTTTGCCTAACTAGTCTCAATTTAGG
ACATGTGTTTTGATTTTCTTCCATTTCTGTCACAATGATGATGGCTGGCGAAAGAA
AAAAAATCTGATCTAAAAATATATATTTAATGCTAAGTTGGAATTTGTAAATCTACAG
TATAATTGGCTCATTTCAACAATTTTTTACCATGTAAATTTGTTGAAGAACATTATTG
TTGTTGAACAATGAAAGAAAAAAATATGGTTGTTAGAAAAAAATGATTTACGATTTT
GCCAAGTGTGCATGCTCTTTCATGGGAAGATATGAATTAATTATCAAAATCTATATA
AAAAAAAGGAAGATAATCTTCATTCTTTCATAACTTAGTTAATAAATTAAATTGATTA
GGATTGGTAACATAGTCAATTCAATTTATCCCGTTAAAGAATGTTATAAATTCGATT
GTTGACCCCTCGTTGAAAATTTGGAATTATGCGGGATGTTTAGAAACTTTGCCATA
AGACCAAAAGATTGGTAGTATTTGATAGTAGTACAAGAGTAATCATTTTTCTTCTTT
AATAACATAAAACGCAGGTGGTAAAAAGGTTCAGCTATGTGGAGTGGCACAGTAC
GTGAATCTCGTAGGGGTCACTATTGGTTACACCATCACTGCCTCCATAAGCTTAGT
GTAAGTCAAAGATTCTGATTTATTTCGATTATTTTGTTATGGTTATACTAACATGTTG
TTCTGAATAAAATTACTAATAATTGTTTGATTGGTGTTTTTGTACGTCTTCGTTAGA
GCGATTGGGAAATCAAATTGTTATCATGACAAGGGACATAAAGCGAAATGTTCTGT
ATCGAATTATCCATACATGGCGGCATTTGGGATCGTCCAGATCATTCTGAGCCAG
CTTCCTAACTTCCACAAGCTCTCTTTCCTATCCATCATCGCCGCGGTTATGTCCTT
CTCTTATGCGTCTATCGGAATAGGCCTAGCCATCGCTACTGTAGCAAGTACATTCC
CCTTCTTTATCTTAAAACATAGTGGTTTATATGGATGATTCTTCAAAGTTGACACTA
ACCGTGAAAATGGTATACAATATATATGAAAGGTGGGAAGATTGGTAAGACAGAAT
TGACAGGGACAGTGATAGGTGTGGACGTAACTGCGTCTGAAAAAGTTTGGAAATT
GTTTCAAGCGATTGGAGACATTGCCTTTTCATACGCTTTTACCACTATTCTCATCGA
GATTCAGGCATGTACTACTGATTCCTACTATCTTCCGTTTACTATTGTTTTCATTTG
CTTGTTATTATTAATTTCGCCAAAGAGAGGTAAATAAGAATACCTTGAAGATAAGA
TGTTATTATTAATTAGACAGTTAGGAAAAAATATAGATGGATGGATGATGGATAAAA
ATAGTTTCATATTTTAGATATGTGAAGCTCTAAAGATAGTGACGCTCTAGTAGTATG
TCTTGTTTATTTTGCAGGACACATTGAGATCAAGCCCACCAGAGAACAAAGTGATG
AAACGAGCAAGTCTTGTCGGAGTCTCAACCACAACTGTTTTCTACATCTTGTGTGG
TTGCATCGGATATGCTGCGTTCGGCAACCAAGCCCCTGGTGACTTCCTTACCGAT
TTTGGTTTTTACGAACCTTATTGGCTCATCGACTTTGCCAATGCTTGCATTGCTCTC
CATCTAATAGGTGCCTATCAGGTATAACTCACAAACAAAAGAATAGGATAAGTGTG
TAACATACATTTACCGTGTTCAAGTTCATTAAAAGTCTCATTATTGTGTTAGAATTTT
TAGCTTTAACAATTCAGAAGATTGTAGAAATGGAGTTATTACTAAATATTGTTTCTA
AAAAATGCTCTTTTTTTTTTTTTATCCCTGTATTATTCGCAGGTGTATGCGCAGCCG
TTTTTCCAGTTTGTTGAGGAAAACTGCAACAAAAAATGGCCTCAAAGCAATTTCAT
CAACAAAGAATACTCGTCAAAGGTTCCTTTGCTTGGAAAATGTCGTATCAACCTCT
TCAGACTGGTTTGGAGGACATGCTATGTTGTTTTGACAACATTTGTAGCAATGATA
TTCCCCTTCTTCAATGCGATCTTGGGTTTGCTAGGGGCACTCGCGTTCTGGCCAC
TCACAGTTTATTTTCCGGTGGCAATGCACATTGCGCAGGCTAAAGTCAAGAAGTAT
TCTCGTAGATGGTTGGCCTTGAACCTCCTCGTATTGGTTTGCTTGATCGTCTCGGC
CCTTGCCGCCGTAGGATCCATCATTGGCTTAATTAATAGTGTCAAGTCATACAAGC
CCTTCAAGAATTTAGACTAGTGTGACTTATAATCTATGTTTGCCAAAAAAAAACCTT
GTGATCCATATGAATTATATGAAATTTATTTGATGCTAAATATTTAGTACTTAATGGTT
TCTCCAAATAATGTGACGTTCTGTTTTCAGCTATGTTAAAAACCAAAATGCTAACTT
ATGTATTAGTACTAAAATTTATGAAAATGTATTAGTTATTGATTTATTTTTAGGACTA
CAATTATTGAATCAACATTGGATGTTTGAGTCCCATGAGATATGGATTTCAGCTTTT
TTCAAATTCGTGTGGTTGTGTCAATTTCGAGTTATTATTATTTATTTTGCTTAATGGA
ATTGTCGGGGAAATCTTGAAAACAGACACTCACAGATTGTGTAATTTATTTGGTTT
GGTGTGTCCTACATAAGTTGCTATCACATCTTATGTATTGGAGGAGTTGGGCAATA
GAGGATCAAGGCAAGTTTGGTTTTCTATTAACGTTTCTACTCTGCATTTGCTTACAA
AGTCATTTTCAAGGTTTTGTGGTCGTATGTCACTTGATGG
SEQ ID NO: 7: AtAAP8^{Col-0} (genomic) (Introns are underlined)
AGGGAGTACTCTAATAAGACGACCTCTGTCAATAACTCTCTTCCCCTCTCTTCTCT
CCTCTGGTTCAGTGGTTCTCTCACAATGATGGACGCATACAACAATCCCTCGGCG
GTGGAGTCGGGTGACGCCGCCGTGAAAAGCGTCGACGACGATGGTCGAGAGAA
GAGAACGGGAACATTTTGGACGGCGAGTGCGCACATAATCACGGCGGTCATAGG
CTCAGGGGTGCTGTCGTTGGCTTGGGCTATAGCACAGCTTGGTTGGGTGGCAGG
AACCACAGTTTTGGTCGCTTTCGCCATCATTACTTACTACACGTCCACCTTGCTCG
CCGACTGTTACCGTTCGCCGGACTCCATCACCGGAACACGCAACTATAATTACAT
GGGCGTCGTCCGATCTTACCTTGGTATGGATTCATATAAACAAATTCATTTTGTGT
CTTTATCAGCATTGTTTTTCACAGATTTTTCAGTTTTCTAGACATTTTTTCTCAGATG
AACAAGGATTTTGTTCATTTGATATCATTTAGATTTTGCCTAACTAGTCTCAATTTAG
GACATGTGTTTTGATTTTCTTCCATTTCTGTCACAATGATGATGGCTGGCGAAAGA
AAAAAAATCTGATCTAAAAATATATATTTAATGCTAAGTTGGAATTTGTAAATCTACA
GTATAATTGGCTCATTTCAACAATTTTTTACCATGTAAATTTGTTGAAGAACATTATT
GTTGTTGAACAATGAAAGAAAAAAATATGGTTGTTAGAAAAAAATGATTTACGATTT
TGCCAAGTGTGCATGCTCTTTCATGGGAAGATATGAATTAATTATCAAAATCTATAT
AAAAAAAAGGAAGATAATCTTCATTCTTTCATAACTTAGTTAATAAATTAAATTGATT
AGGATTGGTAACATAGTCAATTCAATTTATCCCGTTAAAGAATGTTATAAATTCGAT
TGTTGACCCCTCGTTGAAAATTTGGAATTATGCGGGATGTTTAGAAACTTTGCCAT
AAGACCAAAAGATTGGTAGTATTTGATAGTAGTACAAGAGTAATCATTTTTCTTCTT
TAATAACATAAAACGCAGGTGGTAAAAAGGTTCAGCTATGTGGAGTGGCACAGTA
CGTGAATCTCGTAGGGGTCACTATTGGTTACACCATCACTGCCTCCATAAGCTTAG
TGTAAGTCAAAGATTCTGATTTATTTCGATTATTTTGTTATGGTTATACTAACATGTT
GTTCTGAATAAAATTACTAATAATTGTTTGATTGGTGTTTTTGTACGTCTTCGTTAG
AGCGATTGGGAAATCAAATTGTTATCATGACAAGGGAACATAAAGCGAAATGTTCTG
TATCGAATTATCCATACATGGCGGCATTTGGGATCGTCCAGATCATTCTGAGCCAG
CTTCCTAACTTCCACAAGCTCTCTTTCCTATCCATCATCGCCGCGGTTATGTCCTT
CTCTTATGCGTCTATCGGAATAGGCCTAGCCATCGCTACTGTAGCAAGTACATTCC
CCTTCTTTATCTTAAAACATAGTGGTTTATATGGATGATTCTTCAAAGTTGACACTA
ACCGTGAAAATGGTATACAATATATATGAAAGGTGGGAAGATTGGTAAGACAGAAT
TGACAGGGACAGTGATAGGTGTGGACGTAACTGCGTCTGAAAAAGTTTGGAAATT
GTTTCAAGCGATTGGAGACATTGCCTTTTCATACGCTTTTACCACTATTCTCATCGA
GATTCAGGCATGTACTACTGATTCCTACTATCTTCCGTTTACTATTGTTTTCATTTG
CTTGTTATTATTAATTTCGCCAAAGAGAGGTAAAATAAGAATACCTTGAAGATAAGA

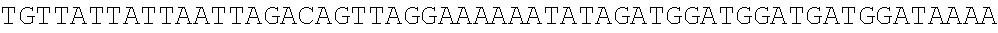

ATAGTTTCATATTTTAGATATGTGAAGCTCTAAAGATAGTGACGCTCTAGTAGTATG
TCTTGTTTATTTTGCAGGACACATTGAGATCAAGCCCACCAGAGAACAAAGTGATG
AAACGAGCAAGTCTTGTCGGAGTCTCAACCACAACTGTTTTCTACATCTTGTGTGG
TTGCATCGGATATGCTGCGTTCGGCAACCAAGCCCCTGGTGACTTCCTTACCGAT
TTTGGTTTTTACGAACCTTATTGGCTCATCGACTTTGCCAATGCTTGCATTGCTCTC
CATCTAATAGGTGCCTATCAGGTATAACTCACAAACAAAAGAATAGGATAAGTGTG
TAACATACATTTACCGTGTTCAAGTTCATTAAAAGTCTCATTATTGTGTTAGAATTTT
TAGCTTTAACAATTCAGAAGATTGTAGAAATGGAGTTATTACTAAATATTGTTTCTA
AAAAATGCTCTTTTTTTTTTTTTATCCCTGTATTATTCGCAGGTGTATGCGCAGCCG
TTTTTCCAGTTTGTTGAGGAAAACTGCAACAAAAAATGGCCTCAAAGCAATTTCAT
CAACAAAGAATACTCGTCAAAGGTTCCTTTGCTTGGAAAATGTCGTGTCAACCTCT
TCAGACTGGTTTGGAGGACATGCTATGTTGTTTTGACAACATTTGTAGCAATGATA
TTCCCCTTCTTCAATGCGATCTTGGGTTTGCTAGGGGCATTCGCGTTCTGGCCAC
TCACAGTTTATTTTCCGGTGGCAATGCACATTGCGCAGGCTAAAGTCAAGAAGTAT
TCTCGTAGATGGTTGGCCTTGAACCTCCTCGTATTGGTTTGCTTGATCGTCTCGGC
CCTTGCCGCCGTAGGATCCATCATTGGCTTAATTAATAGTGTCAAGTCATACAAGC
CCTTCAAGAATTTAGACTAGTGTGACTTATAATCTATGTTTGCCAAAAAAAAACCTT
GTGATCCATATGAATTATATGAAATTTATTTCATGCTAAATATTTAGTACTTAATGTT
TCTCCAAATAATGTGACGTTCTGTTTTCAGCTATGTTAAAAACCAAAATGCTAACTT
ATGTATTAGTACTAAAATTTATGAAAATGTATTAGTTATTGATTTATTTTTAGGACTA
CAATTATTGAATCAACATTGGATGTTTGAGTCCCATGAGATATGGATTTCAGCTTTT
TTCAAATTCGTGTGGTTGTGTCAATTTCGAGTTATTATTATTTATTTTGCTTAATGGA
ATTGTCGGGGAAATCTTGAAAACAGACACTCACAGATTGTGTAATTTATTTGGTTT
GGTGTGTCCTACATAAGTTGCTATCACATCTTATGTATTGGAGGAGTTGGGCAATA
GAGGATCAAGGCAAGTTTGGTTTTCTATTAACGTTTCTACTCTGCATTTGCTTACAA
AGTCATTTTCAAGGTTTTGTGGTCGTATGTCACTTGATGG
SEQ ID NO: 8: AtAAP8 A410 (genomic)
AGGGAGTACTCTAATAAGACGACCTCTGTCAATAACTCTCTTCCCCTCTCTTCTCT
CCTCTGGTTCAGTGGTTCTCTCACAATGATGGACGCATACCACAATCCTTCGGCG
GTGGAGTCGGGTGACGCCGCCGTGAAAAGCGTCGACGACGATGGTCGAGAGAA
GAGAACGGGAACATTTTGGACGGCGAGTGCGCACATAATCACGGCGGTCATAGG
CTCAGGGGTGCTGTCGTTGGCTTGGGCTATAGCACAGCTTGGTTGGGTGGCAGG
AACCACAGTTTTGGTCGCTTTCGCCATCATTACTTACTACACGTCCACCTTGCTCG
CCGACTGTTACCGTTCGCCGGACTCCATCACCGGAACACGCAACTATAATTACAT
GGGCGTCGTCCGATCTTACCTTGGTATGGATTCATATAAACAAATTCATTTTGTGT
CTTTATCAGCATTGTTTTTCACAGATTTTTCAGTTTTCTAGACATTTTTTCTCAGATG
AACAAGGATTTTGTTCATTTGATATCATTTAGATTTTGCCTAACTAGTCTCAATTTAC
GACATGTGTTTTGATTTTCTTCCATTTCTGTCACAATGATGATGGCTGGCGAAAGA
AAAAAAATCTGATCTAAAAATATATATTTAATGCTAAGTTGGAATTTGTAAATCTACA
GTATAATTGGCTCATTTCAACAATTTCTTTCCATGTAAATTTGTTGAAGAACATTATT
GTTGTTGAACAATGAAAGAAAAAAATATGGTTGTTAGAAAAAAATGATTTACGATTT
TGCCAAGTGTGCATGCTCTTTCATGGGAAGATATGAATTAATTATCAAAATCTATAT
AAAAAAAAGGAAGATAATCTTCATTCTTTCATAACTTAGTTAATAAATTAAATTGATT
AGGATTGGTAACATAGTCAATTCAATTTATCCCGTTAAAGAATGTTATAAATTCGAT
TGTTGACCCCTCGTTGAAAATTTGGAATTATGCGGGATGTTTAGAAACTTTGCCAT
AAGACCAAAAGATTGGTAGTATTTGATAGTAGTACAAGAGTAATCATTTTTCTTCTT
TAATAACATAAAACGCAGGTGGTAAAAAGGTTCAGCTATGTGGAGTGGCACAGTA
CGTGAATCTCGTAGGGGTCACTATTGGTTACACCATCACTGCCTCCATAAGCTTAG
TGTAAGTCAAAGATTCTGATTTATTTCGATTATTTTGTTATGGTTATACTAACATGTT
GTTCTGAATAAAATTACTAATAATTGTTTGATTGGTGTTTTTGTACGTCTTCGTTAG
AGCGATTGGGAAATCAAATTGTTATCATGACAAGGGACATAAAGCGAAATGTTCTG
TATCGAATTATCCATACATGGCGGCATTTGGGATCGTCCAGATCATTCTGAGCCAG
CTTCCTAACTTCCACAAGCTCTCTTTCCTATCCATCATCGCCGCGGTTATGTCCTT
CTCTTATGCGTCTATCGGAATAGGCCTAGCCATCGCTACTGTAGCAAGTACATTCC
CCTTCTTTATCTTAAAACATAGTGGTTTATATGGATGATTCTTCAAAGTTGACACTA
ACCGTGAAAATGGTATACAATATATATGAAAGGTGGGAAGATTGGTAAGACAGAAT
TGACAGGGACAGTGATAGGTGTGGACGTAACTGCGTCTGAAAAAGTTTGGAAATT
GTTTCAAGCGATTGGAGACATTGCCTTTTCATACGCTTTTACCACTATTCTCATCGA
GATTCAGGCATGTACTACTGATTCCTACTATCTTCCGTTTACTATTGTTTTCATTTG
CTTGTTATTATTAATTTCGCCAAAAAGAGGTAAAATAAGAATACCTTGAAGATAAGA
TGTTATTATTGATTAGAAAGGTAGGAAAAAATATAGATGGATGGATGATGGATCAA
ATAGTTTCATATTTTAGATATGTGAAGCTCTAAAGATAGTGACGCTCTAGTAGTATG
TCTTGTTTATTTTGCAGGACACATTGAGATCAAGCCCACCAGAGAACAAAGTGATG
AAACGAGCAAGTCTTGCCGGAGTCTCAACCACAACTGTTTTCTACATCTTGTGTGG
TTGCATCGGATATGCTGCGTTCGGCAACCAAGCCCCTGGTGACTTCCTTACCGAT
TTTGGTTTTTACGAACCTTATTGGCTCATCGACTTTGCCAATGCTTGCATTGCTCTC
CATCTAATCGGTGCCTATCAGGTATAACTCACAAACAAAAGAATAGGATAAGTGTG
TAACATACATTTACCGTGTTCAAGTTCATTAAAAGTCTCATTATTGTGTTAGAATTTT
TAGCTTTAACAATTCAGAAGATTGTAGAAATGGAGTTATTACTAAATATTGTTTCTA
AAAAATGCTCTTTTTTTTTTTTTATCCCTGTATTATTCGCAGGTGTATGCGCAGCC
TTTTTCCAGTTTGTTGAGGAAAACTGCAACAAAAAATGGCCTCAAAGCAATTTCAT
CAACAAAGAATACTCGTCAAAGGTTCCTTTGCTTGGAAAATGTCGTGTCAACCTCT
TCAGACTGGTTTGGAGGACATGCTATGTTGTTTTGACAACATTTGTAGCAATGATA
TTCCCCTTCTTCAATGCGATCTTGGGTTTGCTAGGGGCATTCGCGTTCTGGCCAC
TCACAGTTTATTTTCCGGTGGCAATGCACATTGCGCAGGCTAAAGTCAAGAAGTAT
TCTCGTAGATGGTTGGCCTTGAACCTCCTCGTATTGGTTTGCTTGATCGTCTCGGC
CCTTGCCGCCGTAGGATCCATCATTGGCTTAATTAATAGTGTCAAGTCATACAAGC
CCTTCAAGAATTTAGACTAGTGTGACTTATAATCTATGTTTGCCAAAAAAAAACCT
GTGATCCATATGAAATTTATTTCATGCTAAATATTTAGTACTTAATGTTTCTCCAAAT
AATGTGACGTTCTGTTTTCAGCTATGTTAAAAAACAAAATGCTAACTTGTGTATTAG
TACTAAAATTTATGAAAATGTATTAGTTATTGATTTATTTTTAGGACTACAATTATTG
AATCAACATTGGATGTTTGAGTCCCATGAGATATGGATTTCAGCTTTTTTCAAATTC
GTGTGGTTGTGTCAATTTCGAGTTATTATTATTTATTTTGCTTAATGGAATTGTCGG
GGAAATCTTGAAAACAGACACTCACAGATTGTGTAATTTATTTGGTTTGGTGTGTC
CTACATAAGTTGCTATCACATCTTATGTATTGGAGGAGTTGGGCAATAGAGGATCA
AGGCAAGTTTGGTTTTCTATTAACGTTTCTACTCTGCATTTGCTTACAAAGTCATTT
TCAAGGTTTTGTGGTCGTATGTCACTTGATGG
RICE
SEQ ID NO: 9: EEC81471 Osl_24794 [Oryza sativa Indica Group] (protein);

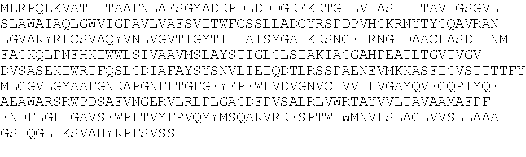

SEQ ID NO: 10: EEC81471 Osl_24794 [Oryza sativa Indica Group] (genomic):
XP_015647443.1
ATGGAGAGGCCGCAAGAGAAGGTGGCCACCACCACCACCGCCGCCTTCAACTC
GCCGAGTCCGGCTACGCCGACCGCCCCGACCTCGACGACGACGGCCGCGAGAA
GCGCACAGGGACGCTGGTGACGGCGAGCGCGCACATAATAACGGCGGTGATCG
GCTCCGGCGTGCTGTCGCTGGCGTGGGCGATAGCGCAGCTGGGGTGGGTGATC
GGGCCGGCCGTGCTGGTGGCGTTCTCGGTCATAACCTGGTTCTGCTCCAGCCTC
CTCGCCGACTGCTACCGATCTCCCGACCCCGTCCATGGCAAGCGCAACTACACC
TACGGCCAAGCCGTCAGGGCCAACCTAGGTGTGGCCAAGTACAGGCTCTGCTCG
GTGGCACAGTACGTCAATCTCGTCGGCGTCACCATTGGCTACACCATCACTACGG
CCATCAGCATGGGTGCGATCAAACGGTCCAACTGCTTCCATCGCAACGGCCACG
ACGCAGCCTGCTTGGCATCTGACACGACCAACATGATCATATTTGCTGGCATCCA
AATCCTCCTCTCGCAGCTGCCGAATTTTCACAAAATTTGGTGGCTCTCCATTGTCG
CTGCTGTCATGTCACTGGCCTACTCAACCATTGGCCTTGGCCTCTCCATTGCAAAA
ATTGCAGGTGGGGCCCACCCCGAGGCAACCCTCACAGGGGTGACTGTTGGAGTG
GATGTGTCTGCAAGTGAGAAAATCTGGAGAACTTTTCAGTCACTTGGTGACATTGC
CTTTGCATACTCCTACTCCAATGTCCTCATAGAAATTCAGGACACGCTGCGGTCGA
GCCCGGCGGAGAACGAGGTGATGAAGAAGGCGTCGTTCATCGGAGTCTCGACGA
CGACGACGTTCTACATGCTGTGCGGCGTGCTCGGCTACGCGGCGTTCGGCAACC
GCGCGCCGGGGAACTTCCTCACCGGCTTCGGCTTCTACGAGCCCTTCTGGCTCG
TCGACGTCGGCAACGTCTGCATCGTCGTCCACCTCGTCGGCGCCTACCAGGTCT
TCTGCCAGCCCATCTACCAGTTCGCCGAGGCCTGGGCGCGCTCGCGCGTGGCCG
GACAGCGCCTTCGTCAACGGCGAGCGCGTGCTCCGGCTGCCGCTCGGCGCCGG
CGACTTCCCCGTCAGCGCGCTCCGCCTCGTCTGGCGCACGGCCTACGTCGTGCT
CACCGCCGTCGCCGCCATGGCGTTCCCCTTCTTCAACGACTTCCTCGGCCTCATC

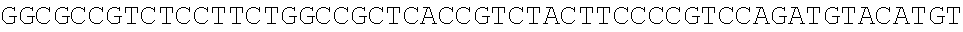

CTCAGGCCAAGGTCCGGCGATTCTCGCCGACGTGGACGTGGATGAACGTGCTCA
GCCTCGCCTGCCTCGTCGTCTCCCTCCTCGCCGCCGCCGGCTCCATCCAGGGCC
TCATCAAATCCGTCGCACATTACAAGCCATTCAGCGTCTCCTCATGA
SEQ ID NO: 11: EEE66520 OsJ_22995 [Oryza sativa Japonica Group] (protein)

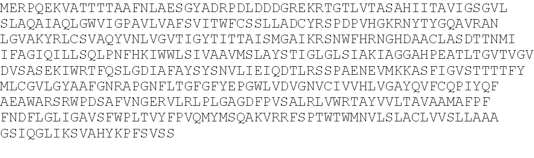

SEQ ID NO: 12: EEE66520 OsJ_22995 [Oryza sativa Japonica Group] (genomic)
ATGGAGAGGCCGCAAGAGAAGGTGGCCACCACCACCACCGCCGCCTTCAACCTC
GCCGAGTCCGGCTACGCCGACCGCCCCGACCTCGACGACGACGGCCGCGAGAA
GCGCACAGGGACGCTGGTGACGGCGAGCGCGCACATAATAACGGCGGTGATCG
GCTCCGGCGTGCTGTCGCTGGCGTGGGCGATAGCGCAGCTGGGGTGGGTGATC
GGGCCGGCCGTGCTGGTGGCGTTCTCGGTCATAACCTGGTTCTGCTCCAGCCTC
CTCGCCGACTGCTACCGATCTCCCGACCCCGTCCATGGCAAGCGCAACTACACC
TACGGCCAAGCCGTCAGGGCCAACCTAGGTGTGGCCAAGTACAGGCTCTGCTCG
GTGGCACAGTACGTCAATCTCGTCGGCGTCACCATTGGCTACACCATCACTACGG
CCATCAGCATGGGTGCGATCAAACGGTCCAACTGGTTCCATCGCAACGGCCACG
ACGCAGCCTGCTTGGCATCTGACACGACCAACATGATCATATTTGCTGGCATCCA
AATCCTCCTCTCGCAGCTGCCGAATTTTCACAAAATTTGGTGGCTCTCCATTGTCG
CTGCTGTCATGTCACTGGCCTACTCAACCATTGGCCTTGGCCTCTCCATTGCAAAA
ATTGCAGGTGGGGCCCACCCCGAGGCAACCCTCACAGGGGTGACTGTTGGAGTG
GATGTGTCTGCAAGTGAGAAAATCTGGAGAACTTTTCAGTCACTTGGTGACATTGC
CTTTGCATACTCCTACTCCAATGTCCTCATAGAAATTCAGGACACGCTGCGGTCGA
GCCCGGCGGAGAACGAGGTGATGAAGAAGGCGTCGTTCATCGGAGTCTCGACGA
CGACGACGTTCTACATGCTGTGCGGCGTGCTCGGCTACGCGGCGTTCGGCAACC
GCGCGCCGGGGAACTTCCTCACCGGCTTCGGCTTCTACGAGCCCTTCTGGCTCG
TCGACGTCGGCAACGTCTGCATCGTCGTCCACCTCGTCGGCGCCTACCAGGTCT
TCTGCCAGCCCATCTACCAGTTCGCCGAGGCCTGGGCGCGCTCGCGGTGGCCG
GACAGCGCCTTCGTCAACGGCGAGCGCGTGCTCCGGCTGCCGCTCGGCGCCGG
CGACTTCCCCGTCAGCGCGCTCCGCCTCGTCTGGCGCACGGCCTACGTCGTGCT
CACCGCCGTCGCCGCCATGGCGTTCCCCCTTCTTCAACGACTTCCTCGGCCTCATC

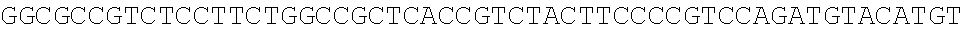

CTCAGGCCAAGGTCCGGCGATTCTCGCCGACGTGGACGTGGATGAACGTGCTCA
GCCTCGCCTGCCTCGTCGTCTCCCTCCTCGCCGCCGCCGGCTCCATCCAGGGCC
TCATCAAATCCGTCGCACATTACAAGCCATTCAGCGTCTCCTCATGA
SEQ ID NO: 13: XP_015647443 AAP6 [Oryza sativa Japonica Group] (protein)
MGMERPQEKVATTTTTAAFNLAESGYADRPDLDDDGREKRTGTLVTASAHIITAVIGSG
VLSLAWAIAQLGWVIGPAVLVAFSVITWFCSSLLADCYRSPDPVHGKRNYTYGQAVRA
NLGVAKYRLCSVAQYYVNLVGVTIGYTITTAISMGAIKRSNWFHRNGHDAACLASDTTN
MIIFAGIQILLSQLPNFHKIWWLSIVAAVMSLAYSTIGLGLSIAKIAGGAHPEATLTGVTV
GVDVSASEKIWRTFQSLGDIAFAYSYSNVLIEIQDTLRSSPAENEVMKKASFIGVSTTTT
FYMLCGVLGYAAFGNRAPGNFLTGFGFYEPFWLVDVGNVCIVVHLVGAYQFCQPIY
QFAEAWARSRWPDSAFVNGERVLRLPLGAGDFPVSALRLVWRTAYVVLTAVAAMAF

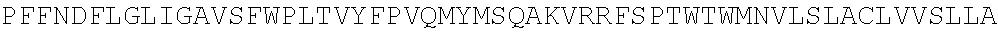

AAGSIQGLIKSVAHYKPFSVSS
SEQ ID NO: 14: XP_015647443 AAP6 [Oryza sativa Japonica Group] (genomic)
ATGGGGATGGAGAGGCCGCAAGAGAAGGTGGCCACCACCACCACCGCCGCCTT
CAACCTCGCCGAGTCCGGCTACGCCGACCGCCCCGACCTCGACGACGACGGCC
GCGAGAAGCGCACAGGGACGCTGGTGACGGCGAGCGCGCACATAATAACGGCG
GTGATCGGCTCCGGCGTGCTGTCGCTGGCGTGGGCGATAGCGCAGCTGGGGTG
GGTGATCGGGCCGGCCGTGCTGGTGGCGTTCTCGGTCATAACCTGGTTCTGCTC
CAGCCTCCTCGCCGACTGCTACCGATCTCCCGACCCCGTCCATGGCAAGCGCAA
CTACACCTACGGCCAAGCCGTCAGGGCCAACCTAGGTGTGGCCAAGTACAGGCT
CTGCTCGGTGGCACAGTACGTCAATCTCGTCGGCGTCACCATTGGCTACACCATC
ACTACGGCCATCAGCATGGGTGCGATCAAACGGTCCAACTGGTTCCATCGCAACG
GCCACGACGCAGCCTGCTTGGCATCTGACACGACCAACATGATCATATTTGCTGG
CATCCAAATCCTCCTCTCGCAGCTGCCGAATTTTCACAAAATTTGGTGGCTCTCCA
TTGTCGCTGCTGTCATGTCACTGGCCTACTCAACCATTGGCCTTGGCCTCTCCATT
GCAAAAATTGCAGGTGGGGCCCACCCCGAGGCAACCCTCACAGGGGTGACTGTT
GGAGTGGATGTGTCTGCAAGTGAGAAAATCTGGAGAACTTTTCAGTCACTTGGTG
ACATTGCCTTTGCATACTCCTACTCCAATGTCCTCATAGAAATTCAGGACACGCTG
CGGTCGAGCCCGGCGGAGAACGAGGTGATGAAGAAGGCGTCGTTCATCGGAGT
CTCGACGACGACGACGTTCTACATGCTGTGCGGCGTGCTCGGCTACGCGGCGTT
CGGCAACCGCGCGCCGGGGAACTTCCTCACCGGCTTCGGCTTCTACGAGCCCTT
CTGGCTCGTCGACGTCGGCAACGTCTGCATCGTCGTCCACCTCGTCGGCGCCTA
CCAGGTCTTCTGCCAGCCCATCTACCAGTTCGCCGAGGCCTGGGCGCGCTCGCG
GTGGCCGGACAGCGCCTTCGTCAACGGCGAGCGCGTGCTCCGGCTGCCGCTCG
GCGCCGGCGACTTCCCCGTCAGCGCGCTCCGCCTCGTCTGGCGCACGGCCTAC
GTCGTGCTCACCGCCGTCGCCGCCATGGCGTTCCCCTTCTTCAACGACTTCCTCG

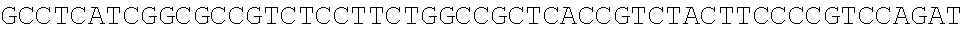

GTACATGTCTCAGGCCAAGGTCCGGCGATTCTCGCCGACGTGGACGTGGATGAA
CGTGCTCAGCCTCGCCTGCCTCGTCGTCTCCCTCCTCGCCGCCGCCGGCTCCAT
CCAGGGCCTCATCAAATCCGTCGCACATTACAAGCCATTCAGCGTCTCCTCATGA
SEQ ID NO: 15: BAC82953.1 putative amino acid permease [Oryza sativa Japonica
Group] (protein)
MAAAGRTLGCIYAGTLVTASAHIITAVIGSGVLSLAWAIAQLGWVIGPAVLVAFSVITWF
CSSLLADCYRSPDPVHGKRNYTYGQAVRANLGVAKYRLCSVAQYVNLVGVTIGYTITT
AISMGAIKRSNWFHRNGHDAACLASDTTNMIIFAGIQILLSQLPNFHKIWWLSIVAAVMS
LAYSTIGLGLSIAKIAGGAHPEATLTGVTVGVDVSASEKIWRTFQSLGDIAFAYSYSNVL
IEIQDTLRSSPAENEVMKKASFIGVSTTTTFYMLCGVLGYAAFGNRAPGNFLTGFGFY
EPFWLVDVGNVCIVVHLVGAYQVFCQPIYQFAEAWARSRWPDSAFVNGERVLRL

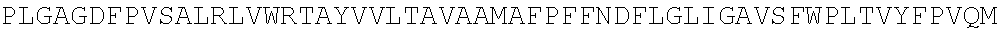

YMSQAKVRRFSPTWTWMNVLSLACLVVSLLAAAGSIQGLIKSVAHYKPFSVSS
SEQ ID NO: 16: BAC82953.1 putative amino acid permease [Oryza sativa Japonica
Group] (genomic)
ATGGCGGCGGCCGGACGAACACTTGGATGCATATATGCAGGGACGCTGGTGACG
GCGAGCGCGCACATAATAACGGCGGTGATCGGCTCCGGCGTGCTGTCGCTGGC
GTGGGCGATAGCGCAGCTGGGGTGGGTGATCGGGCCGGCCGTGCTGGTGGCGT
TCTCGGTCATAACCTGGTTCTGCTCCAGCCTCCTCGCCGACTGCTACCGATCTCC
CGACCCCGTCCATGGCAAGCGCAACTACACCTACGGCCAAGCCGTCAGGGCCAA
CCTAGGTGTGGCCAAGTACAGGCTCTGCTCGGTGGCACAGTACGTCAATCTCGTC
GGCGTCACCATTGGCTACACCATCACTACGGCCATCAGCATGGGTGCGATCAAAC
GGTCCAACTGGTTCCATCGCAACGGCCACGACGCAGCCTGCTTGGCATCTGACA
CGACCAACATGATCATATTTGCTGGCATCCAAATCCTCCTCTCGCAGCTGCCGAAT
TTTCACAAAATTTGGTGGCTCTCCATTGTCCGCTGCTGTCATGTCACTGGCCTACTC
AACCATTGGCCTTGGCCTCTCCATTGCAAAAATTGCAGGTGGGGCCCACCCCGAG
GCAACCCTCACAGGGGTGACTGTTGGAGTGGATGTGTCTGCAAGTGAGAAAATCT
GGAGAACTTTTCAGTCACTTGGTGACATTGCCTTTGCATACTCCTACTCCAATGTC
CTCATAGAAATTCAGGACACGCTGCGGTCGAGCCCGGCGGAGAACGAGGTGATG
AAGAAGGCGTCGTTCATCGGAGTCTCGACGACGACGACGTTCTACATGCTGTGC
GGCGTGCTCGGCTACGCGGCGTTCGGCAACCGCGCGCCGGGGAACTTCCTCAC
CGGCTTCGGCTTCTACGAGCCCTTCTGGCTCGTCGACGTCGGCAACGTCTGCAT
CGTCGTCCACCTCGTCGGCGCCTACCAGGTCTTCTGCCAGCCCATCTACCAGTT
CGCCGAGGCCTGGGCGCGCTCGCGGTGGCCGGACAGCGCCTTCGTCAACGGCG
AGCGCGTGCTCCGGCTGCCGCTCGGCGCCGGCGACTTCCCCGTCAGCGCGCTC
CGCCTCGTCTGGCGCACGGCCTACGTCGTGCTCACCGCCGTCGCCGCCATGGC

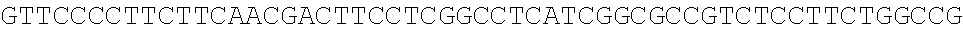

CTCACCGTCTACTTCCCCGTCCAGATGTACATGTCTCAGGCCAAGGTCCGGCGAT
TCTCGCCGACGTGGACGTGGATGAACGTGCTCAGCCTCGCCTGCCTCGTCGTCT
CCCTCCTCGCCGCCGCCGGCTCCATCCAGGGCCTCATCAAATCCGTCGCACATT
ACAAGCCATTCAGCGTCTCCTCATGA
SEQ ID NO: 17: XP_015644123.1 amino acid permease 3 [Oryza sativa Japonica
Group] (protein)
MAKDVEMAVRNGDGGGGGGYYATHPHGGAGGEDVDDDGKQRRTGNVWTASAHIIT
AVIGSGVLSLAWATAQLGWVVGPVTLMLFALITYYTSGLLADCYRTGDPVSGKRNYTY
MDAVAAYLGGWQVWSCGVFQYVNLVGTAIGYTITASISAAAVHKANCYHKNGHDAD
CGVYDTTMIVFGVVQIFFSMLPNFSDLSWLSILAAVMSFSYSTIAVGLSLARTISGATG
KTTLTGVEVGVDVTSAQKIWLAFQALGDIAFAYSYSMILIEIQDTVKSPPAENKTMKKAT
LLGVSTTTAFYMLCGCLGYAAFGNAAPGNMLTGFGFYEPYWLIDFANVCIVVHLVGAY
QVFCQPIFAAVETFAARRWPGSEFITRERPVVAGRSFSVNMFRLTWRTAFVVVSTVL

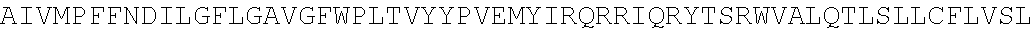

ASAVASIEGVSESLKHYVPFKTKS
SEQ ID NO: 18: XP_015644123.1 amino acid permease 3 [Oryza sativa Japonica
Group] (genomic)
ATGGCGAAGGACGTGGAGATGGCGGTGCGGAACGGAGACGGCGGCGGCGGCG
GCGGCTACTACGCCACCCACCCGCACGGCGGCGCCGGCGGCGAGGACGTCGAC
GACGACGGCAAGCAGCGGCGAACCGGTAACGTATGGACGGCGAGCGCGCACAT
CATCACGGCGGTGATCGGCTCCGGCGTGCTCTCTCTCGCATGGGCAACGGCG
CAGCTCGGCTGGGTGGTCGGGCCGGTGACTCTGATGCTCTTCGCCCTCATCACG
TACTACACCTCTGGGCTCCTCGCCGACTGCTACCGCACTGGCGATCCGGTCAGC
GGCAAGCGCAACTACACCTACATGGATGCCGTTGCGGCCTACTTAGGTGGCTGG
CAAGTCTGGTCCTGTGGTGTTTTCCAATATGTCAACCTGGTTGGGACAGCAATTG
GGTACACAATCACAGCATCCATCAGCGCAGCGGCTGTGCACAAGGCCAACTGCT
ACCACAAGAACGGCCACGATGCCGATTGCGGTGTCTACGACACCACGTACATGAT
CGTCTTTGGAGTCGTCCAGATCTTCTTCTCCATGCTGCCCAACTTCAGTGACCTCT
CATGGCTTTCCATCCTCGCCGCGGTCATGTCATTCTCATACTCGACCATTGCCGTT
GGCCTCTCGCTTGCGCGAACAATATCAGGTGCTACTGGTAAGACTACTCTGA
CTGGCGTTGAGGTTGGAGTTGACGTCACTTCAGCCCAGAAGATCTGGCTCGCGTT
CCAAGCGCTCGGTGACATCGCGTTCGCCTACTCCTACTCCATGATCCTTATAGAA
ATTCAGGACACGGTGAAGTCTCCACCGGCGGAGAACAAGACGATGAAGAAGGCA
ACGCTGCTGGGGGTGCGACCACGACGGCGTTCTACATGCTGTGCGGGTGCCTG
GGGTACGCGGCGTTCGGGAACGCGGCGCCGGGGAACATGCTCACCGGGTTCGG
CTTCTACGAGCCCTACTGGCTGATCGACTTCGCCAACGTCTGCATCGTGGTCCAC
CTGGTCGGCGCCTACCAGGTGTTCTGCCAGCCCATCTTCGCCGCCGTCGAGACG
TTCGCCGCCAGGCGGTGGCCGGGCTCGGAGTTCATCACCCGGGAGCGCCCCGT
CGTGGCCGGCAGGTCGTTCAGCGTCAACATGTTCAGGCTGACGTGGCGGACGGC
GTTCGTGGTCGTCAGCACGGTGCTCGCCATCGTGATGCCCTTCTTCAACGACATC

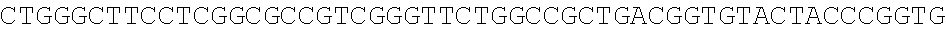

GAGATGTACATCCGGCAGCGGCGGATACAGCGGTACACGTCCAGGTGGGTGGC
GCTGCAGACGCTCAGCCTCCTCTGCTTCCTCGTCTCGCTCGCCTCCGCCGTCGC
CTCCATCGAGGGCGTCAGCGAGTCGCTCAAGCACTACGTCCCCTTCAAGACCAA
GTCGTGA
SEQ ID NO: 19: EEC68963.1 hypothetical protein Osl_37697 [Oryza sativa Indica
Group] (protein)
MSDMASGQKAKQQVMKPMEVSVEAGNAGDAAWLDDDGRARRTGTFWTASAHIITVI
GSGVLSLAWAIAQLGWVAGPAVMLLFAFVIYYTSTLLAECYRTGDPATGKRNYTYMD
AVRANLGGAKVTFCGVIQYANLVGVAIGYTIASSISMRAIRRAGCFHHNGHGDPCRSS
SNPYMILFGVVQIVFSQIPDFDQIWWLSIVAAVMSFTYSGIGLSLGIVQTISNGGIQGSL
TGISIGVGVSSTQKVWRSLQAFGDIAFAYSFSNILIEIQDTIKAPPPSEAKVMKSATRLS
VATTTVFYMLCGCMGYAAFGDAAPDNLLTGFGFYEPFWLLDVANVAIVVHLVGAYQV
FVQPIFAFVERWASRRWPDSAFIAKELRVGPFALSLFRLTWRSAFVCLTTVVAMLLPF

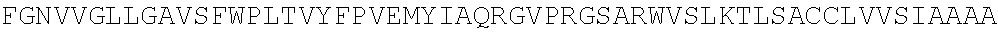

GSIADVIDALKVYRPFSG
SEQ ID NO: 20: EEC68963.1 hyptothetical protein Osl_37697 [Oryza sativa Indica
Group] (genomic)
ATGTCCGACATGGCGTCGGGGCAGAAGGCGAAGCAGCAGGTGATGAAGCCGAT
GGAGGTGTCGGTGGAGGCCGGGAACGCCGGGGATGCGGCGTGGCTGGACGAC
GACGGGCGGGCGCGGCGGACGGGCACGTTCTGGACGGCGAGCGCGCACATCAT
CACCGCCGTCATCGGCTCCGGCGTGCTGTCGCTGGCGTGGGCGATCGCGCAG
CTCGGGTGGGTGGCCGGCCCCGCCGTGATGCTCCTCTTCGCCTTCGTCATCTAC
TACACCTCCACCCTCCTCGCCGAGTGCTACCGCACCGGCGACCCGGCCACCGGC
AAGCGCAACTACACCTACATGGACGCCGTGCGCGCCAACCTCGGCGGCGCCAAG
GTCACCTTCTGCGGCGTCATCCAGTACGCCAACCTCGTCGGCGTCGCCATCGGC
TACACCATCGCGTCGTCCATCAGCATGCGCGCCATCAGGAGGGCCGGCTGCTTC
CACCACAACGGCCATGGTGACCCGTGCCGCAGCTCCAGCAACCCTTACATGATC
CTCTTCGGCGTCGTGCAGATCGTCTTCTCGCAGATCCCGGACTTCGACCAGATTT
GGTGGCTGTCCATCGTCGCCGCCGTCATGTCCTTCACCTACTCCGGCATCGGCC
TCTCCCTCGGCATCGTCCAGACAATCTCCAATGGCGGGATCCAGGGCAGCCTCA
CCGGAATCAGCATCGGCGTCGGCGTCAGCTCAACGCAGAAGGTGTGGCGCAGC
CTGCAGGCATTCGGCGACATCGCCTTCGCATACTCCTTCTCCAACATCCTCATCG
AGATCCAAGACACGATCAAGGCGCCGCCGCCGTCGGAGGCGAAGGTGATGAAGA
GCGCGACGAGGCTGAGCGTGGCGACGACCACGGTGTTCTACATGCTGTGCGGG
TGCATGGGCTACGCGGCGTTCGGCGACGCGGCGCCCGACAACCTCCTCACGGG
CTTCGGCTTCTACGAGCCCTTCTGGCTGCTCGACGTCGCCAACGTCGCCATCGTC
GTGCACCTCGTCGGCGCCTACCAGGTGTTCGTCCAGCCAATCTTCGCCTTCGTCG
AGCGCTGGGCCTCCCGCCGGTGGCCGGACAGCGCGTTCATCGCCAAGGAGCTC
CGCGTGGGGCCCTTCGCGCTCAGCCTCTTCCGCCTGACGTGGCGCTCGGCGTTC
GTCTGCCTCACCACAGTCGTCGCCATGCTCCTCCCCTTCTTCGGCAACGTGGTGG

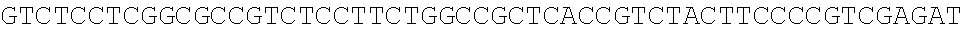

GTACATCGCGCAGCGCGGCGTGCCACGTGGCAGCGCGAGGTGGGTCTCGCTCA
AGACGCTCAGCGCGTGCTGCCTCGTCGTCTCCATCGCCGCCGCCGCGGGCTCCA
TTGCTGACGTCATCGACGCTCTCAAGGTGTACAGACCGTTCAGCGGATGA
SEQ ID NO: 21: EAY82481.1 hypothetical protein Osl_37698 [Oryza sativa Indica
Group] (protein)
MASGQKVVKPMEVSVEAGNAGEAAWLDDDGRARRTGTFWTASAHIITAVIGSGVLSL
AWAIAQLGWVAGPAVMLLFAFVIYYTSTLLAECYRTGDPATGKRNYTYMDAVRANLG
GAKVTFCGVIQYANLVGVAIGYTIASSISMRAIRRAGCFHHNGHGDPCRSSSNPYMILF
GVVQIVFSQPIDFDQIWWLSIVAAVMSFTYSGIGLSLGIVQTISNGGIQGSLTGISIGVGV
SSTQKVWRSLQAFGDIAFAYSFSNILIEIQDTIKAPPPSEAKVMKSATRLSVATTTVFYM
LCGCMGYAAFGDAAPDNLLTGFGFYEPFWLLDVANVAIVVHLVGAYQVFVQPIFAFVE
RWASRRWPDSAFIAKELRVGPFALSLFRLTWRSAFVCLTTVVAMLLPFFGNVVGLLG

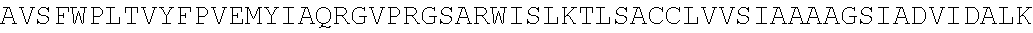

VYRPFSG
SEQ ID NO: 22: EAY82481.1 hypothetical protein Osl_37698
[Oryza sativa Indica Group] (genomic)
ATGGCGTCGGGGCAGAAGGTGGTGAAGCCGATGGAGGTGTCGGTGGAGGCCGG
GAACGCCGGGGAGGCGGCGTGGCTGGACGACGACGGGCGGGCGCGGCGGACG
GGCACGTTCTGGACGGCGAGCGCGCACATCATCACCGCCGTCATCGGCTCCGGC
GTGCTGTCGCTGGCGTGGGCGATCGCGCAGCTGGGCTGGGTGGCCGGCCCC
GCCGTGATGCTCCTCTTCGCCTTCGTCATCTACTACACCTCCACCCTCCTCGCCG
AGTGCTACCGCACCGGCGACCCGGCCACCGGCAAGCGCAACTACACCTACATGG
ACGCCGTGCGCGCCAACCTCGGCGGCGCCAAGGTCACCTTCTGCGGCGTCATCC
AGTACGCCAACCTCGTCGGCGTCGCCATCGGCTACACCATCGCGTCGTCCATCA
GCATGCGCGCCATCAGGAGGGCCGGCTGCTTCCACCACAACGGCCATGGTGACC
CGTGCCGCAGCTCCAGCAACCCTTACATGATCCTCTTCGGCGTCGTGCAGATCGT
CTTCTCCCAGATCCCTGACTTCGACCAGATTTGGTGGCTGTCCATCGTCGCCGCC
GTCATGTCCTTCACCTACTCCGGCATCGGCCTCTCCCTCGGCATCGTCCAGACTA
TCTCCAATGGCGGGATCCAGGGCAGCCTCACCGGCATCAGCATCGGAGTCGGCG
TCAGCTCGACGCAGAAGGTGTGGCGCAGCTTGCAGGCATTCGGCGACATCGCCT
TCGCATACTCCTTCTCCAACATCCTCATCGAGATCCAAGACACGATCAAGGCGCC
GCCGCCGTCGGAGGCGAAGGTGATGAAGAGCGCGACGAGGCTGAGCGTGGCGA
CGACCACGGTGTTCTACATGCTGTGCGGGTGCATGGGCTACGCGGCGTTCGGCG
ACGCGGCGCCCGACAACCTCCTCACCGGCTTCGGGTTCTACGAGCCCTTCTGGC
TGCTCGACGTCGCCAACGTCGCCATCGTCGTGCACCTCGTCGGCGCCTACCAGG
TGTTCGTCCAGCCAATCTTCGCCTTCGTCGAGCGCTGGGCCTCCCGCCGGTGGC
CGGACAGCGCGTTCATCGCCAAGGAGCTCCGCGTGGGGCCCTTCGCGCTCAGC
CTCTTCCGCCTGACGTGGCGCTCGGCGTTCGTCTGCCTCACCACCGTCGTCGCC

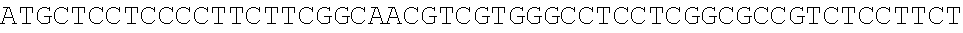

GGCCGCTCACCGTCTACTTCCCCGTCGAGATGTACATCGCGCAGCGTGGCGTGC
CGCGGGGGAGCGCGAGGTGGATCTCCCTCAAGACGCTCAGCGCGTGCTGCCTA
GTCGTCTCCATCGCCGCCGCGGCGGGCTCCATTGCTGACGTCATCGACGCGCT
CAAGGTGTACAGACCCTTCAGCGGATGA
SEQ ID NO: 23: BAD53557 putative amino acid carrier [Oryza
sativa Japonica Group] (protein)
MDVYLPRTQGDVDDDGKERRTGTVWTATAHIITAVIGSGVLSLAWAMAQLGWVAGPI
TLLLFAAITFYTCGLLSDCYRVGDPATGKRNYTYTDAVKSYLGGWHVWFCGFCQYVN
MFGTGIGYTITASISAAAINKSNCYHWRGHGTDCSQNTSAYIIGFGVLQALFCQLPNFH
QLWWLSIIAAVMSFSYAAIAVGLSLAQTIMDPLGRTTLTGTVVGVDVDATQKVWLTFQ
ALGNVAFAYSYAIILIEIQDTLRSPPPENATMRRATAAGISTTTGFYLLCGCLGYSAFGN
AAPGNILTGFGFYEPYWLVDVANACIVVHLVGGFQVFCQPLFAAVEGGVARRCPGLL

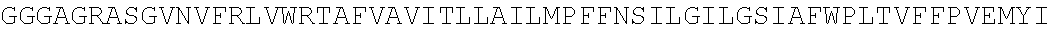

RQRQLPRFSAKWWVALQSLSLVCFLVTVAACAASIQGVLDSLKTYVPFKTRS
SEQ ID NO: 24: BAD53557 putative amino acid carrier
[Oryza sativa Japonica Group] (genomic)
ATGGACGTCTACCTTCCCCGGACCCAAGGCGACGTCGACGACGACGGCAAGGAG
AGGAGGACAGGGACGGTGTGGACGGCGACGGCGCACATAATCACGGCGGTGAT
CGGGTCCGGCGTGCTGTCGCTGGCGTGGGCGATGGCGCAGCTGGGGTGGGTG
GCTGGCCCCATCACCCTCCTCCTCTTCGCCGCCATCACCTTCTACACCTGCGGCC
TCCTCTCCGACTGCTACCGCGTCGGCGACCCGGCCACCGGCAAGCGCAACTACA
CCTACACCGACGCCGTCAAGTCCTACCTCGGTGGCTGGCACGTCTGGTTCTGCG
GCTTCTGCCAGTACGTCAACATGTTCGGCACCGGCATCGGCTACACCATCACCGC
CTCCATCTCCGCCGCGGCTATCAACAAGTCCAACTGCTACCACTGGCGCGGCCAT
GGCACGGACTGCAGCCAGAACACGAGCGCCTACATCATCGGCTTCGGCGTCCTG
CAGGCCCTCTTCTGCCAGCTCCCAAACTTCCACCAGCTCTGGTGGCTGTCCATCA
TCGCCGCCGTCATGTCCTTCTCGTACGCCGCCATCGCCGTCGGCTTGTCGCTGG
CGCAGACCATCATGGACCCGCTGGGGAGGACGACGCTGACGGGCACGGTGGTC
GGCGTCGACGTCGACGCCACGCAGAAGGTGTGGCTCACGTTCCAGGCGCTGG
GGAACGTCGCCTTCGCCTACTCCTACGCCATCATCCTCATCGAGATCCAGGACAC
GCTGCGGTCGCCGCCGCCGGAGAACGCGACGATGCGGCGCGCCACGGCGGCG
GGGATCTCGACGACCACGGGGTTCTACCTGCTGTGCGGCTGCCTGGGCTACTCG
GCGTTCGGGAACGCGGCGCCGGGCAACATCCTCACCGGCTTCGGCTTCTACGAG
CCATACTGGCTGGTGGACGTGGCCAACGCCTGCATCGTGGTGCACCTCGTCGGC
GGGTTCCAGGTGTTCTGCCAGCCGCTGTTCGCCGCCGTGGAGGGCGGCGTGGC
GCGGCGGTGCCCGGGGCTGCTCGGCGGCGGCGCGGGGCGGGCCAGCGGCGT
GAACGTGTTCCGGCTTGTGTGGAGGACGGCGTTCGTGGCGGTGATCACGCTGCT

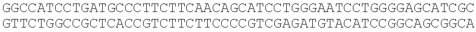

GCTGCCGCGGTTCAGCGCCAAGTGGGTGGCGCTGCAGAGCCTGAGCCTCGTCT
GCTTCCTCGTCACCGTCGCCGCCTGCGCCGCCTCCATCCAGGGCGTCCTCGACT
CGCTCAAGACCTACGTGCCCTTCAAGACCAGGTCCTGA
SEQ ID NO: 25: XP_015637472.1 AAP3 isoform X2 [Oryza
sativa Japonica Group] (protein)
MGENGVVASKLCYPAAAMEVVAAELGHTAGSKLYDDDGRLKRTGTMWTASAHIITAV
IGSGVLSLGWAIAQLGWVAGPAVMLLFSFVTYYTSALLADCYRSGDESTGKRNYTYM
DAVNANLSGIKVQVCGFLQYANIVGVAIGYTIAASISMLAIKRANCFHVEGHGDPCNISS
TPYMIIFGVAEIFFSQIPDFDQISWLSILAAVMSFTYSTIGLGLGVVQVVANGGVKGSLT
GISIGVVTPMDKVWRSLQAFGDIAFAYSYSLILIEIQDTIRAPPPSESRVMRRATVVSVA
VTTLFYMLCGCTGYAAFGDAAPGNLLTGFGFYEPFWLLDVANAAIVVHLVGAYQVY
CQPLFAFVEKWAQQRWPKSWYITKDIDVPLSLSGGGGGGGRCYKLNLFRLTWRSAF

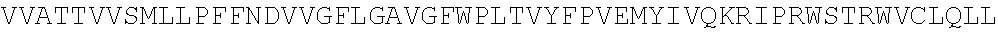

SLACLAITVASAAGSIAGILSDLKVYKPFATTY
SEQ ID NO: 26: XP_015637472.1 AAP3 isoform X2 [Oryza
sativa Japonica Group] (genomic)
ATGGGGGAGAACGGTGTGGTGGCGAGCAAGCTGTGCTACCCGGCGGCGGCCAT
GGAGGTGGTCGCCGCCGAGCTCGGCCACACGGCCGGCTCCAAGCTGTACGACG
ACGACGGCCGCCTCAAGCGCACCGGGACGATGTGGACGGCGAGCGCGCACATC
ATCACGGCGGTGATCGGCTCCGGCGTGCTGTCGCTGGGGTGGGCGATCGCGCA
GCTGGGTTGGGTGGCCGGCCCCGCCGTCATGCTGCTCTTCTCGTTCGTCACCTA
CTACACCTCCGCGCTGCTCGCCGACTGCTACCGCTCCGGCGACGAGAGCACCGG
CAAGCGCAACTACACCTACATGGACGCCGTGAACGCCAACCTGAGTGGCATCAA
GGTCCAGGTCTGCGGGTTCCTGCAGTACGCCAACATCGTCGGCGTCGCCATCGG
CTACACCATTGCCGCCTCCATTAGCATGCTGGCGATCAAGCGGGCGAACTGCTTC
CACGTCGAGGGGCACGGCGACCCGTGCAACATCTCGAGCACGCCGTACATGATC
ATCTTCGGCGTGGCGGAGATCTTCTTCTCGCAGATCCCGGACTTCGACCAGATCT
CGTGGCTGTCCATCCTCGCCGCCGTCATGTCGTTCACCTACTCCACCATCGGGCT
CGGCCTCGGCGTCGTGCAGGTGGTGGCCAACGGCGGCGTCAAGGGGAGCCTCA
CCGGGATCAGCATCGGCGTGGTGACGCCCATGGACAAGGTGTGGCGGAGCCTG
CAGGCGTTCGGCGACATCGCCTTCGCCTACTCCTACTCCCTCATCCTCATCGAGA
TCCAGGACACCATCCGGGCGCCGCCGCCGTCGGAGTCGAGGGTGATGCGGCGC
GCCACCGTGGTGAGCGTCGCCGTCACCACGCTCTTCTACATGCTCTGCGGCTGC
ACGGGGTACGCGGCGTTCGGCGACGCCGCGCCGGGCAACCTCCTCACCGGGTT
CGGCTTCTACGAGCCCTTCTGGCTCCTCGACGTTGCCAACGCCGCCATCGTCGT
CCACCTCGTCGGCGCCTACCAGGTCTACTGCCAGCCGCTGTTCGCCTTCGTCGA
GAAGTGGGCGCAGCAGCGGTGGCCGAAATCATGGTACATCACCAAGGATATCGA
CGTGCCGCTCTCCCTCTCCGGCGGCGGCGGCGGCGGCGGAAGGTGCTACAAGC
TGAACCTGTTCAGGCTGACATGGAGGTCGGCGTTCGTGGTGGCGACGACGGTGG

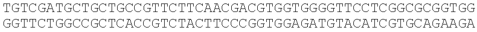

GGATACCGAGGTGGAGCACGCGGTGGGTGTGCCTGCAGCTGCTCAGCCTCGCC
TGCCTCGCCATCACCGTCGCCTCCGCCGCCGGCTCCATCGCCGGAATCCTCTCC
GACCTCAAGGTCTACAAGCCGTTCGCCACCACCTACTAA
SEQ ID NO: 27: XP_025881587 AAP3 isoform X1 [Oryza
sativa Japonica Group] (protein)
MTHHTKFNPNYISICNPASSLSLIFTSLFLNWKRVRGSRRGDFCKEMGENGVVASKLC
YPAAAMEVVAAELGHTAGSKLYDDDGRLKRTGTMWTASAHIITAVIGSGVLSLGWAIA
QLGWVAGPAVMLLFSFVTYYTSALLADCYRSGDESTGKRNYTYMDAVNANLSGIKVQ
VCGFLQYANIVGVAIGYTIAASISMLAIKRANCFHVEGHGDPCNISSTPYMIIFGVAEIFF
SQIPDFDQISWLSILAAVMSFTYSTIGLGLGVVQVVANGGVKGSLTGISIGVVTPMDKV
WRSLQAFGDIAFAYSYSLILIEIQDTIRAPPPSESRVMRRATVVSVAVTTLFYMLCG
CTGYAAFGDAAPGNLLTGFGFYEPFWLLDVANAAIVVHLVGAYQVYCQPLFAFVEKW
AQQRWPKSWWYITKDIDVPLSLSGGGGGGGRCYKLNLFRLTWRSAFVVATTVVSMLLP

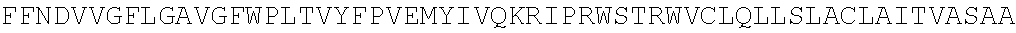

GSIAGILSDLKVYKPFATTY
SEQ ID NO: 28: XP_025881587 AAP3 X1 [Oryza sativa Japonica Group]
(genomic)
ATGACACACCACACCAAGTTCAACCCCAACTATATCTCTATTTGTAACCCTGCTTC
TTCTCTCTCTTTGATCTTCACTTCTCTCTTCCTCAATTGGAAGAGGGTTAGGGAT
CAAGAAGAGGAGACTTTTGCAAAGAGATGGGGGAGAACGGTGTGGTGGCGAGCA
AGCTGTGCTACCCGGCGGCGGCCATGGAGGTGGTCGCCGCCGAGCTCGGCCAC
ACCGGCCGGCTCCAAGCTGTACGACGACGACGGCCGCCTCAAGCGCACCGGGAC
GATGTGGACGGCGAGCGCGCACATCATCACGGCGGTGATCGGCTCCGGCGTGC
TGTCGCTGGGGTGGGCGATCGCGCAGCTGGGTTGGGTGGCCGGCCCCGCCGTC
ATGCTGCTCTTCTCGTTCGTCACCTACTACACCTCCGCGCTGCTCGCCGACTGCT
ACCGCTCCGGCGACGAGAGCACCGGCAAGCGCAACTACACCTACATGGACGCCG
TGAACGCCAACCTGAGTGGCATCAAGGTCCAGGTCTGCGGGTTCCTGCAGTACG
CCAACATCGTCGGCGTCGCCATCGGCTACACCATTGCCGCCTCCATTAGCATGCT
GGCGATCAAGCGGGCGAACTGCTTCCACGTCGAGGGGCACGGCGACCCGTGCA
ACATCTCGAGCACGCCGTACATGATCATCTTCGGCGTGGCGGAGATCTTCTTCTC
GCAGATCCCGGACTTCGACCAGATCTCGTGGCTGTCCATCCTCGCCGCCGTCAT
GTCGTTCACCTACTCCACCATCGGGCTCGGCCTCGGCGTCGTGCAGGTGGTGGC
CAACGGCGGCGTCAAGGGGAGCCTCACCGGGATCAGCATCGGCGTGGTGACGC
CCATGGACAAGGTGTGGCGGAGCCTGCAGGCGTTCGGCGACATCGCCTTCGCCT
ACTCCTACTCCCTCATCCTCATCGAGATCCAGGACACCATCCGGGCGCCGCCGC
CGTCGGAGTCGAGGGTGATGCGGCGCGCCACCGTGGTGAGCGTCGCCGTCACC
ACGCTCTTCTACATGCTCTGCGGCTGCACGGGGTACGCGGCGTTCGGCGACGCC
GCGCCGGGCAACCTCCTCACCGGGTTCGGCTTCTACGAGCCCTTCTGGCTCCTC
GACGTTGCCAACGCCGCCATCGTCGTCCACCTCGTCGGCGCCTACCAGGTCTAC
TGCCAGCCGCTGTTCGCCTTCGTCGAGAAGTGGGCGCAGCAGCGGTGGCCGAAA
TCATGGTACATCACCAAGGATATCGACGTGCCGCTCTCCCTCTCCGGCGGCGGC
GGCGGCGGCGGAAGGTGCTACAAGCTGAACCTGTTCAGGCTGACATGGAGGTCG
GCGTTCGTGGTGGCGACGACGGTGGTGTCGATGCTGCTGCCGTTCTTCAACGAC

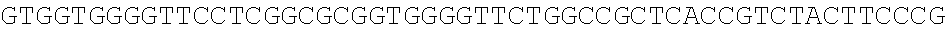

GTGGAGATGTACATCGTGCAGAAGAGGATACCGAGGTGGAGCACGCGGTGGGTG
TGCCTGCAGCTGCTCAGCCTCGCCTGCCTCGCCATCACCGTCGCCTCCGCCGCC
GGCTCCATCGCCGGAATCCTCTCCGACCTCAAGGTCTACAAGCCGTTCGCCACCA
CCTACTAA
SEQ ID NO: 29: XP_015629427 AAP4 [Oryza sativa Japonica Group] (protein)
MGENVVGTYYYPPSAAAMDGVELGHAAAGSKLFDDDGRPRRNGTMWWTASAHIITAVI
GSGVLSLGWAIAQLGWVAGPAVMVLFSLVTYYTSSLLSDCYRSGDPVTGKRNYTYM
DAVNANLSGFKVKICGFLQYANIVGVAIGYTIAASISMLAIGRANCFHRKGHGDPCNVS
SVPYMIVFGVAEVFFSQIPDFDQISWLSMLAAVMSFTYSVIGLSLGIVQVVANGGLKGS
LTGISIGVVTPMDKVWRSLQAFGDIAFAYSYSLILIEIQDTIRAPPPSESAVMKRATVVS
VAVTTVFMLCGSMGYAAFGDDAPGNLLTGFGFYEPFWLLDIANAAIVVHLVGAYQVF
CQPLFAFVEKWAAQRWPESPYITGEVELRLSPSSRRCRVNLFRSTWRTAFVVATTVV

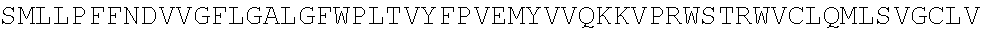

ISIAAAAGSIAGVMSDLKVYRPFKGY
SEQ ID NO: 30: XP_015629427 AAP4 [Oryza sativa Japonica Group] (genomic)
ATGGGGGAGAACGTGGTTGGCACGTACTACTACCCGCCTTCGGCGGCCGCCATG
GACGGCGTGGAGCTCGGCCACGCCGCCGCCGGCTCCAAGCTCTTCGACGACGA
CGGCCGCCCCAGGCGCAACGGGACGATGTGGACGGCGAGCGCGCACATCATCA
CGGCGGTGATCGGCTCCGGCGTGCTGTCGCTGGGGTGGGCCATCGCGCAGCTC
GGCTGGGTGGCCGGGCCGGCGGTCATGGTGCTCTTCTCCCTCGTCACCTACTAC
ACCTCATCCCTCCTCTCCGATTGCTACCGCTCCGGCGACCCCGTCACCGGCAAG
CGGAACTACACCTACATGGACGCCGTGAACGCCAACCTGAGCGGGTTCAAGGTG
AAGATCTGCGGGTTCTTGCAGTACGCCAACATCGTCGGCGTCGCCATCGGCTACA
CCATCGCGGCGTCCATCAGCATGCTGGCGATCGGGAGGGCCAACTGCTTCCACA
GGAAGGGGCACGGCGACCCGTGCAACGTCTCCAGCGTGCCCTACATGATCGTCT
TCGGCGTCGCCGAGGTCTTCTTCTCGCAGATCCCCGACTTCGATCAGATCTCCTG
GCTCTCCATGCTCGCCGCCGTCATGTCCTTCACCTACTCCGTCATCGGCCTCAGC
CTCGGCATCGTCCAAGTCGTCGCGAACGGAGGGTTGAAGGGAAGCCTGACCG
GGATCAGCATCGGCGTGGTGACGCCGATGGACAAGGTGTGGAGGAGCCTGCAG
GCGTTCGGCGACATCGCGTTCGCCTACTCCTACTCGCTGATCCTCATCGAGATCC
AGGACACCATCCGGGCGCCGCCGCCGTCGGAGTCGGCGGTGATGAAGCGCGCC
ACGGTGGTGAGCGTGGCGGTGACCACGGTGTTCTACATGCTCTGCGGCAGCATG
GGGTACGCGGCGTTCGGCGACGACGCGCCGGGGAACCTCCTCACCGGGTTCGG
CTTCTACGAGCCCTTCTGGCTCCTCGACATCGCCAACGCCGCCATCGTCGTCCAC
CTCGTCGGCGCCTACCAGGTGTTCTGCCAGCCGCTCTTCGCCTTCGTCGAGAAG
TGGGCGGCGCAGCGGTGGCCGGAGTCGCCGTACATCACCGGGGAGGTGGAGCT
CCGCCTCTCGCCGTCGTCGAGGCGGTGCAGGGTGAACCTGTTCCGGTCGACGTG
GCGCACGGCGTTCGTCGTCGCCACCACGGTGGTGTCCATGCTGCTGCCCTTCTT

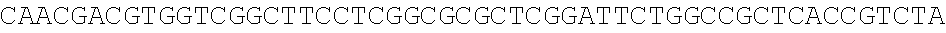

CTTCCCCGTGGAGATGTACGTGGTGCAGAAGAAGGTGCCACGGTGGAGCACACG
GTGGGTGTGCCTGCAGATGCTCAGCGTCGGCTGCCTCGTCATCTCCATCGCCGC
CGCCGCGGGCTCCATCGCCGGCGTCATGTCGGATCTCAAGGTTTACCGCCCGTT
CAAGGGTTACTGA
SOYBEAN
SEQ ID NO: 31: KHN37208 AAP6 [Glycine soja] (protein)
MFVETPEDGGKNFDDDGRVKRTGTWITASAHIITAVIGSGVLSLAWAIAQMGWVAGP
AVLFAFSFITYFTSTLLADCYRSPDPVHGKRNYTYSDVVRSVLGGRKFQLCGLAQYINL
VGVTIGYTITASISMVAVKRSNCFHKHGHHDKCYTSNNPFMILFACIQIVLSQIPNFHKL
WWLSIVAAVMSFAYSSIGLGLSVAKVAGGGEPVRTTLTGVQVGVDVTGSEKVWRTF
QAIGDIAFAYAYSNVLIEIQDTLKSSPPENKVMKRASLIGILTTTLFYVLCGCLGYAAFGN
DAPGNFLTGFGFYEPFWLIDFANICIAVHLVGAYQVFCQPIFGFVENWGKERWPNSHF

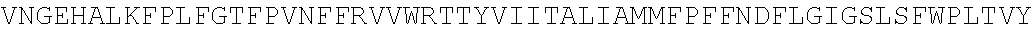

FPIEMYIKQSKMQKFSFTWTWLKILSWACLIVSIISAAGSIQGLAQDLKKYQPFKAQQ
SEQ ID NO: 32: KHN37208 AAP6 [Glycine soja] (genomic)
ATGTTCGTAGAAACCCCTGAAGATGGTGGCAAAAACTTCGACGATGATGGACGAG
TCAAAAGAACTGGTACATGGATAACTGCGAGTGCCCATATCATAACGGCAGTGAT
AGGTTCTGGAGTGTTGTCACTTGCATGGGCAATTGCACAAATGGGTTGGGTGGCA
GGCCCTGCGGTTCTCTTTGCCTTCTCTTTCATCACATACTTCACCTCCACTCTTCTT
GCCGACTGTTATCGTTCACCTGACCCTGTTCATGGCAAGCGAAACTACACCTATTC
AGATGTTGTCAGATCCGTGTTAGGAGGTAGGAAATTTCAGCTGTGTGGATTAGCT
CAGTACATAAATCTTGTCGGTGTAACTATCGGTTACACGATAACGGCTTCAATTAG
TATGGTGGCGGTGAAGAGGTCCAACTGTTTTCACAAACATGGTCATCATGATAAGT
GCTACACGTCAAACAACCCTTTCATGATCCTCTTTGCCTGCATTCAAATCGTGCTT
AGTCAAATACCAAATTTCCATAAGCTTTGGTGGCTCTCCATTGTTGCAGCAGTTAT
GTCTTTTGCTTATTCTTCCATTGGCCTTGGGCTCTCCGTAGCTAAAGTGGCAGGTG
GTGGAGAACCTGTACGGACAACCTTAACGGGGGTGCAAGTTGGGGTGGACGTTA
CGGGATCCGAGAAGGTCTGGAGGACGTTTCAAGCTATTGGTGATATTGCCTTCGC
TTACGCTTATTCTAACGTGCTCATTGAGATACAGGATACCCTGAAATCGAGCCCTC
CAGAAAACAAGGTCATGAAAAGAGCAAGTTTGATTGGCATCTTGACTACAACCTTG
TTCTATGTGCTATGTGGCTGCCTAGGTTATGCAGCATTTGGAAACGACGCACCAG
GAAATTTCCTCACAGGGTTCGGTTTCTACGAGCCCTTTTGGCTAATAGACTTTGCT
AACATCTGCATAGCCGTACACTTGGTTGGAGCATATCAGGTCTTCTGTCAGCCCAT
ATTTGGGTTCGTAGAGAACTGGGGTAAGGAAAGGTGGCCCAATAGCCATTTTGTA
AATGGAGAACACGCTTTAAAGTTTCCACTATTTGGAACCTTCCCTGTGAACTTTTTC
AGGGTGGTATGGAGAACAACATATGTCATCATCACTGCTTTGATAGCTATGATGTT

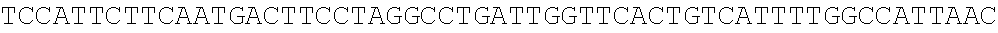

GGTTTACTTCCCCATAGAGATGTACATTAAGCAGTCAAAGATGCAAAAGTTTTCCT
TCACTTGGACATGGCTCAAGATATTGAGCTGGGCTTGCTTGATCGTTTCTATTATC
TCAGCTGCTGGCTCCATCCAAGGCCTCGCTCAAGATCTCAAGAAATATCAGCCCT
TCAAAGCCCAGCAATAA
SEQ ID NO: 33: XP_003526513 AAP6 [Glycine max] (protein)
MNPDQFQKNSMFVETPEDGGKNFDDDGRVKRTGTWITASAHIITAVIGSGVLSLAWAI
AQMGWVAGPAVLFAFSSFITYFTSTLLADCYRSPDPVHGKRNYTYSDVVRSVLGGRKF
QLCGLAQYINLVGVTIGYTITASISMVAVKRSNCFHKHGHHDKCYTSNNPFMILFACIQI
VLSQIPNFHKLWWLSIVAAVMSFAYSSIGLGLSVAKVAGGGEPVRTTLTGVQVGVDVT
GSEKVWRTFQAIGDIAFAYAYSNVLIEIQDTLKSSPPENKVMKRASLIGILTTTLFYVLC
GCLGYAAFGNDAPGNFLTGFGFYEPFWLIDFANICIAVHLVGAYQVFCQPIFGFVENW
GKERWPNSHFVNGEHALKFPLFGTFPVNFFRVVWRTTYVIITALIAMMFPFFNDFLGLI

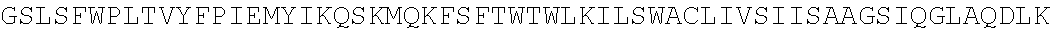

KYQPFKAQQ
SEQ ID NO: 34: XP_003526513 AAP6 [Glycine max] (genomic)
ATGAATCCTGATCAGTTTCAGAAGAACAGCATGTTCGTAGAAACCCCTGAAGATG
GTGGCAAAAACTTCGACGATGATGGACGAGTCAAAAGAACTGGTACATGGATAAC
TGCGAGTGCCCATATCATAACGGCAGTGATAGGTTCTGGAGTGTTGTCACTTGCA
TGGGCAATTGCACAAATGGGTTGGGTGGCAGGCCCTGCGGTTCTCTTTGCCTTCT
CTTTCATCACATACTTCACCTCCACTCTTCTTGCCGACTGTTATCGTTCACCTGAC
CCTGTTCATGGCAAGCGAAACTACACCTATTCAGATGTTGTCAGATCCGTGTTAGG
AGGTAGGAAATTTCAGCTGTGTGGATTAGCTCAGTACATAAATCTTGTCGGTGTAA
CTATCGGTTACACGATAACGGCTTCAATTAGTATGGTGGCGGTGAAGAGGTCCAA
CTGTTTTCACAAACATGGTCATCATGATAAGTGCTACACGTCAAACAACCCTTTCA
TGATCCTCTTTGCCTGCATTCAAATCGTGCTTAGTCAAATACCAAATTTCCATAAGC
TTTGGTGGCTCTCCATTGTTGCAGCAGTTATGTCTTTTGCTTATTCTTCCATTGGCC
TTGGGCTCTCCGTAGCTAAAGTGGCAGGTGGTGGAGAACCTGTACGGACAACCTT
AACGGGGGGTGCAAGTTGGGGTGGACGTTACGGGATCCGAGAAGGTCTGGAGGA
CGTTTCAAGCTATTGGTGATATTGCCTTCGCTTACGCTTATTCTAACGTGCTCATT
GAGATACAGGATACCCTGAAATCGAGCCCTCCAGAAAACAAGGTCATGAAAAGAG
CAAGTTTGATTGGCATCTTGACTACAACCTTGTTCTATGTGCTATGTGGCTGCCTA
GGTTATGCAGCATTTGGAAACGACGCACCAGGAAATTTCCTCACAGGGTTCGGTT
TCTACGAGCCCTTTTGGCTAATAGACTTTGCTAACATCTGCATAGCCGTACACTTG
GTTGGAGCATATCAGGTCTTCTGTCAGCCCATATTTGGGTTCGTAGAGAACTGGG
GTAAGGAAAGGTGGCCCAATAGCCATTTTGTAAATGGAGAACACGCTTTAAAGTTT
CCACTATTTGGAACCTTCCCTGTGAACTTTTTCAGGGTGGTATGGAGAACAACATA
TGTCATCATCACTGCTTTGATAGCTATGATGTTTCCATTCTTCAATGACTTCCTAGG

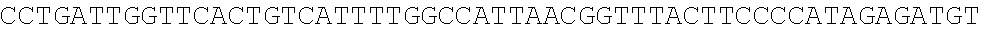

ACATTAAGCAGTCAAAGATGCAAAAGTTTTCCTTCACTTGGACATGGCTCAAGATA
TTGAGCTGGGCTTGCTTGATCGTTTCTATTATCTCAGCTGCTGGCTCCATCCAAGG
CCTCGCTCAAGATCTCAAGAAATATCAGCCCTTCAAAGCCCAGCAATAA
SEQ ID NO: 35: NP_001242816 LOC100777963 [Glycine max] (protein)
MNSDQFQKNSMFVETPEDGGKNFDDDGRVRRTGTWITASAHIITAVIGSGVLSLAWAI
AQMGWVAGPAVLFAFSFITYFTSTLLADCYRSPDPVHGKRNYTYSDVVRSVLGGRKF
QLCGLAQYINLVGVTIGYTITASISMVAVKRSNCFHKHGHHVKCYTSNNPFMILFACIQI
VLSQIPNFHKLWWLSIVAAVMSFAYSSIGLGLSVAKVAGGGEPVRTTLTGVQVGVDVT
GSEKVWRTFQAIGDIAFAYAYSNVLIEIQDTLKSSPPENKVMKRASLIGILTTTLFYVLC
GCLGYAAFGNDAPGNFLTGFGFYEPFWLIDFANICIAVHLVGAYQVFCQPIFGFVENW
GRERWPNSQFVNGEHALNFPLCGTFPVNFFRVVWRTTYVIITALIAMMFPFFNDFLGLI

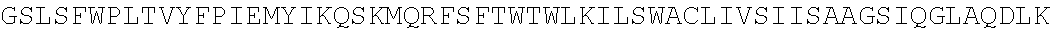

KYQPFKAQQ
SEQ ID NO: 36: NP_001242816 LOC100777963 [Glycine max] (genomic)
ATGAATTCTGATCAGTTTCAGAAGAACAGCATGTTCGTAGAAACCCCTGAAGATGG
TGGCAAAAACTTCGACGATGATGGACGAGTCAGAAGAACGGGTACATGGATAACT
GCGAGTGCCCATATCATAACGGCAGTGATAGGGTCAGGAGTGTTGTCACTTGCAT
GGGCAATTGCACAAATGGGTTGGGTGGCTGGCCCTGCCGTTCTCTTTGCCTTCTC
TTTCATCACTTACTTCCTTCCACTCTTCTTGCCGACTGTTATCGTTCACCTGATCC
TGTTCATGGCAAGCGAAACTACACCTATTCCGATGTTGTCAGATCCGTCTTAGGAG
GGAGGAAATTTCAGCTGTGTGGATTAGCTCAGTACATAAATCTTGTCGGTGTAACT
ATCGGTTACACGATAACGGCTTCAATTAGTATGGTGGCGGTGAAGAGGTCGAATT
GTTTTCACAAACATGGTCATCATGTTAAGTGCTATACGTCAAACAACCCTTTCATGA
TCCTCTTTGCCTGCATTCAAATCGTGCTTAGCCAGATACCAAATTTCCATAAGCTC
TGGTGGCTCTCCATTGTTGCAGCAGTTATGTCTTTTGCTTATTCTTCCATTGGCCT
CGGGCTCTCAGTAGCTAAAGTGGCAGGTGGTGGAGAGCCTGTACGGACAACCTT
AACGGGGGGTGCAAGTTGGGGTAGACGTTACAGGATCCGAGAAGGTCTGGAGGAC
GTTTCAAGCTATTGGTGACATTGCCTTCGCTTATGCTTATTCTAACGTGCTCATCG
AGATACAGGATACCCTGAAATCGAGCCCTCCAGAGAACAAGGTCATGAAAAGAGC
AAGTTTGATTGGCATCTTGACTACAACCTTGTTCTATGTGCTATGTGGCTGCCTAG
GTTATGCAGCATTTGGAAACGATGCACCAGGAAATTTCCTTACAGGGTTCGGCTT
CTACGAGCCCTTTTGGCTCATAGACTTTGCCTAACATCTGCATAGCCGTGCACTTGG
TTGGAGCATATCAGGTCTTCTGTCAGCCCATATTTGGGTTCGTAGAGAACTGGGG
TAGGGAAAGGTGGCCAAATAGCCAATTTGTAAATGGAGAACACGCTTTGAACTTTC
CACTATGTGGAACCTTCCCTGTGAACTTCTTCAGGGTGGTGTGGAGAACAACATA
TGTCATCATCACTGCTTTGATAGCTATGATGTTTCCATTCTTCAATGACTTCCTAGG

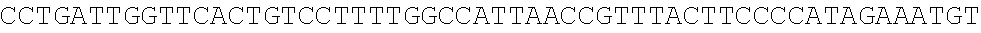

ACATTAAGCAGTCAAAGATGCAAAGGTTTTCCTTCACGTGGACGTGGCTCAAGAT
CTGAGCTGGGCTTGCTTGATCGTTTCTATTATCTCAGCTGCTGGTTCCATCCAAGG
CCTCGCTCAAGATCTCAAGAAATATCAGCCCTTCAAAGCCCAGCAATAA
SEQ ID NO: 37: XP_028228300 AAP6-like [Glycine soja] (protein)
MNSDQFQKNSMFVETPEDGGKNFDDDGRVRRTGTWWITASAHIITAVIGSGVLSLAWAI
AQMGWVAGPAVLFAFSFITYFTSTLLADCYRSPDPVHGKRNYTYSDVVRSVLGGRKF
QLCGLAQYINLVGVTIGYTITASISMVAVKRSSNCFHKHGHHVKCYTSNNPFMILFACIQI
VLSQIPNFHKLWWLSIVAAVMSFAYSSIGLGLSVAKVAGGGEPVRTTLTGVQVGVDVT
GSEKVWRTFQAIGDIAFAYAYSNVLIEIQDTLKSSPPENKVMKRASLIGILTTTLFYVLC
GCLGYAAFGNDAPGNFLTGFGFYEPFWLIDFANICIAVHLVGAYQVFCQPIFGFVENW
GKERWPNSQFVNGEHALNFPLCGTFPVNFFRVVWRTTYVIITALIAMMFPFFNDFLGLI

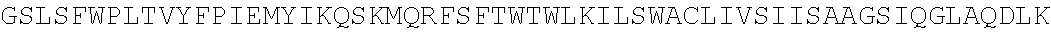

KYQPFKAQQ
SEQ ID NO: 38: XP_028228300 AAP6-like [Glycine soja] (genomic)
ATGAATTCTGATCAGTTTCAGAAGAACAGCATGTTCGTAGAAACCCCTGAAGATGG
TGGCAAAAACTTCGACGATGATGGACGAGTCAGAAGAACTGGTACATGGATAACT
GCGAGTGCCCATATCATAACGGCAGTGATAGGGTCAGGAGTGTTGTCACTTGCAT
GGGCAATTGCACAAATGGGTTGGGTGGCTGGCCCTGCCGTTCTCTTTGCCTTCTC
TTTCATCACTTACTTCACTTCCACTCTTCTTGCCGACTGTTATCGTTCACCTGATCC
TGTTCATGGCAAGCGAAACTACACCTATTCCGATGTTGTCAGATCCGTCTTAGGAG
GGAGGAAATTTCAGCTGTGTGGATTAGCTCAGTACATAAATCTTGTCGGTGTAACT
ATCGGTTACACGATAACGGCTTCAATTAGTATGGTGGCGGTGAAGAGGTCGAATT
GTTTTCACAAACATGGTCATCATGTTAAGTGCTATACGTCAAACAACCCTTTCATGA
TCCTCTTTGCCTGCATTCAAATCGTGCTTAGCCAGATACCAAATTTCCATAAGCTC
TGGTGGCTCTCCATTGTTGCAGCAGTTATGTCTTTTGCTTATTCTTCCATTGGCCT
CGGGCTCTCAGTAGCTAAAGTGGCAGGTGGTGGAGAGCCTGTACGGACAACCTT
AACGGGGGTGCAAGTTGGGGTAGACGTTACAGGATCCGAGAAGGTCTGGAGGAC
GTTTCAAGCTATTGGTGACATTGCCTTCGCTTATGCTTATTCTAACGTGCTCATCG
AGATACAGGATACCCTGAAATCGAGCCCTCCAGAGAACAAGGTCATGAAAAGAGC
AAGTTTGATTGGCATCTTGACTACAACCTTGTTCTATGTGCTATGTGGCTGCCTAG
GTTATGCAGCATTTGGAAACGATGCACCAGGAAATTTCCTCACAGGGTTCGGCTT
CTACGAGCCCTTTTGGCTCATAGACTTTGCTAACATCTGCATAGCCGTGCACTTGG
TTGGAGCATATCAGGTCTTCTGTCAGCCCATATTTGGGTTCGTAGAGAACTGGGG
TAAGGAAAGGTGGCCCAATAGCCAATTTGTAAATGGAGAACACGCTTTGAACTTTC
CACTATGTGGAACCTTCCCTGTGAACTTCTTCAGGGTGGTGTGGAGAACAACATA
TGTCATCATCACTGCTTTGATAGCTATGATGTTTCCATTCTTCAATGACTTCCTAGG

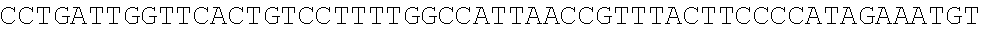

ACATTAAGCAGTCAAAGATGCAAAGGTTTTCCTTCACGTGGACATGGCTCAAGATA
CTGAGCTGGGCTTGCTTGATCGTTTITCTATTATCTCAGCTGCTGGTTCCATCCAAGG
CCTCGCTCAAGATCTCAAGAAATATCAACCCTTCAAAGCCCAGCAATAA
SEQ ID NO: 39: KRH353636.1 hypothetical protein GLYMA_10G255300 [Glycine max]
(protein)
MAVIRSNCFHKYGHEAKCHTSNYPYMTIFAVIQILLSSQIPDFQELSGLSIIAAVMSFGYS
SIGIGLSIAKIAGGNDAKTSLTGLIVGEDVTSQEKLWNTFQAIGNIAFAYAFSQVLVEIQD
TLKSSSPPENQAMKKATLAGCSITSLFYMLCGLLGYAAFGNKAPGNFLTGFGFYEPYW
LVDIGNVFVFVHLVGAYQVFTQPVFQLVETWVAKRWPESNFMGKEYRVGKFRFNGF

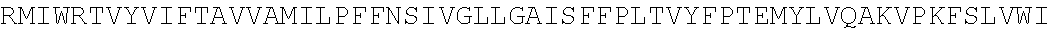

GVKILSGFCLIVTLVAAAGSIQGIIADLKIYEPFK
SEQ ID NO: 40: KRH353636.1 hypothetical protein GLYMA_10G255300 [Glycine max]
(genomic)
ATGGCTGTCATAAGATCGAATTGCTTTCACAAGTATGGGCACGAAGCGAAGTGTC
ATACATCAAATTACCCATATATGACCATCTTTGCGGTCATACAGATTTTATTAAGCC
AAATCCCTGATTTCCAGGAACTCTCAGGCCTCTCTATTATTGCTGCCGTCATGTCT
TTTGGTTATTCTTCCATAGGCATTGGTCTCTCCATAGCCAAAATTGCAGGAGGAAA
CGATGCCAAGACAAGTCTAACGGGGCTCATCGTTGGAGAAGACGTGACAAGCCA
GGAGAAACTATGGAACACTTTCCAAGCAATTGGAAACATTGCTTTTGCATACGCCT
TCAGTCAAGTACTTGTTGAGATACAGGACACGTTAAAATCAAGCCCACCAGAAAAT
CAAGCCATGAAAAAGGCAACCCTTGCTGGATGCTCGATCACCTCACTGTTTTATAT
GTTATGTGGCCTATTAGGCTATGCAGCATTCGGGAACAAGGCACCCGGAAACTTC
TTAACAGGATTTGGGTTTTATGAACCATATTGGCTTGTTGACATTGGTAATGTCTTC
GTATTTGTTCATTTAGTGGGCGCCTACCAGGTATTCACACAACCAGTTTTCCAGCT
TGTGGAAACTTGGGTTGCGAAGCGTTGGCCTGAAAGCAACTTCATGGGAAAAGAA
TATCGTGTTGGCAAGTTCAGATTCAATGGATTCAGGATCATATGGAGGACAGTGTA
CGTGATTTTCACAGCAGTGGTTGCTATGATACTTCCCTTCTTCAACAGCATTGTGG

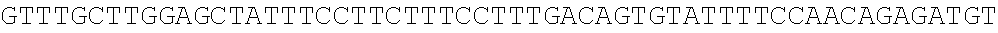

ATCTGGTGCAGGCTAAAGTGCCCAAGTTTTCTCTGGTCTGGATTGGGGTCAAAT
TCTAAGTGGCTTCTGCTTGATTGTCACTCTTGTTGCTGCAGCTGGATCAATCCAAG
GAATCATCGCAGACCTTAAAATCTATGAGCCCTTCAAGTAA
SEQ ID NO: 41: XP_028192809.1 AAP4-like [Glycine soja] (protein)
MLPRSRTLPSRIHQGIIEERHDVRPYVQVEVRPNNIQTETQAMNIQSNYSKCFDDDGR
LKRTGTFWTATAHIITAVIGSGVLSLAWAVAQLGWWVAGPVVMFLFAVVNLYTSNLLTQ
CYRTGDSVNGHRNYTYMEAVKSILGGKKVKLCGLIQYINLFGVAIGYTIAASVSMMAIK
RSNCYHSSHGKDPCHMSSNGYMITFGIAEVIFSQIPDFDQVWWLSIVAAIMSFTYSSV
GLSLGVAKVAENKTFKGSLMGISIGTVTQAGTVTSTQKIWRSLQALGAMAFAYSFSIILI
EIQDTIKSPPAEHKTMRKATTLSIAVTTVFYLLCGCMGYAAFGDNAPGNLLTGFGFYNP
YWLLDIANLAIVIHLVGAYQVFSQPLFAFVEKWWSVRKWPKSNFVTAEYDIPIPCFGVYQ

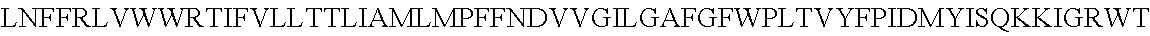

SRWIGLQLLSVSCLIISLLAAVGSMGVVLDLKTYKPFKTSY
SEQ ID NO: 42: XP_028192809.1 AAP4-like [Glycine soja] (genomic)
ATGTTGCCAAGAAGTAGAACCCTTCCTAGCAGAATCCACCAAGGAATTATAGAAGA
GAGGCACGATGTCAGGCCCTACGTACAAGTAGAAGTGCGACCCAATAATATCCAA
ACGGAGACCCAAGCGATGAATATCCAGTCTAACTATTCCAAGTGCTTCGATGATG
ATGGTCGCTTGAAGAGAACAGGAACATTTTGGACGGCAACTGCTCATATCATCAC
TGCTGTGATAGGGTCGGGAGTCCTTTCACTAGCATGGGCGGTTGCTCAGCTTGGT
TGGGTTGCTGGACCTGTTGTCATGTTTCTCTTTGCCGTCGTCAATCTCTACACTTC
CAACCTATTAACACAGTGTTACAGGACCGGTGACTCCGTTAATGGGCACAGAAAT
TACACCTACATGGAGGCTGTCAAGTCCATCTTGGGAGGAAAAAAGGTCAAGTTAT
GTGGCCTCATCCAATATATCAATCTGTTTGGAGTTGCAATCGGGTACACCATTGCT
GCCTCTGTCAGTATGATGGCCATAAAAAGGTCGAATTGCTATCACAGCAGTCATG
GAAAGATCCCTGCCACATGTCAAGCAATGGGTATATGATAACATTTGGAATAGCA
GAAGTGATATTTTCCCAAATCCCAGACTTTGATCAGGTGTGGTGGCTATCCATAGT
TGCAGCTATCATGTCCTTCACTTATTCTTCAGTTGGATTGAGTCTTGGAGTGGCCA
AAGTAGCAGAAAATAAAACTTTCAAAGGAAGCCTGATGGGAATTAGCATTGGCACA
GTAACACAAGCCGGAACAGTCACCAGCACACAGAAAATATGGAGGAGTTTACAAG
CTCTTGGGGCAATGGCCTTTGCATACTCCTTTTCCATTATCCTCATCGAAATTCAG
GACACCATAAAATCTCCTCCTGCAGAGCACAAGACCATGAGAAAGGCCACAACAT
TGAGCATCGCGGTTACCACAGTGTTCTATTTACTCTGTGGATGCATGGGTTATGCA
GCCTTCGGAGATAATGCACCTGGAAATCTCTTGACTGGTTTTGGGTTCTATAACCC
TTATTGGCTTCTGGACATTGCCAACCTTGCAATTGTTATCCACCTAGTTGGGGCAT
ATCAGGTTTTTCCCAGCCCTTATTTGCATTTGTGGAAAAATGGAGTGTACGCAAA
TGGCCAAAGAGCAATTTTGTCACGGCAGAATATGATATACCGATTCCCTGCTTTGG
TGTGTACCAACTCAACTTCTTCCGCTTAGTATGGAGAACCATTTTTGTGCTGTTGA
CGACCCTCATAGCCATGCTCATGCCTTTTTCAACGATGTGGTTGGAATACTTGGC

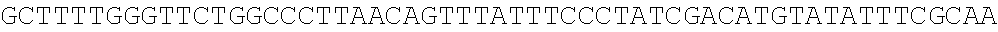

AAGAAGATTGGACGATGGACTAGTCGGTGGATTGGACTTCAATTACTTAGTGTCA
GTTGCCTCATCATTTCATTGTTAGCTGCAGTTGGTTCCATGGCAGGGGTTGTTTTG
GACCTCAAGACTTATAAGCCATTTAAAACTAGTTATTAA
SEQ ID NO: 43: XP_00659084.1; XP_003540867; AAP4 [Glycine max] (protein)
MLPRSRTLPSRIHQGIIEERHNVRHYLQVEVRPNNTQTETEAMNIQSNYSKCFDDDGR
LKRTGTFWMATAHIITAVIGSGVLSLAWAVAQLGWVAGPIVMFLFAVVNLYTSNLLTQC
YRTGDSVTGHRNYTYMEAVNSILGGKKVKLCGLIQYINLFGVAIGYTIAASVSMMAIKR
SNCYHSSHGKDPCHMSSNGYMITFGIAEVIFSQIPDFDQVWWLSIVAAIMSFTYSSVG
LSLGVAKVAENKSFKGSLMGISIGTVTQAGTVTSTQKIWRSLQALGAMAFAYSFSIILIEI
QDTIKSPPAEHKTMRKATTLSIAVTTVFYLLCGCMGYAAFGDNAPGNLLTGFGFYNPY
WLLDIANLAIVIHLVGAYQVFSQPLFAFVEKWWSARKWPKSNFVTAEYDIPIPCFGVYQL

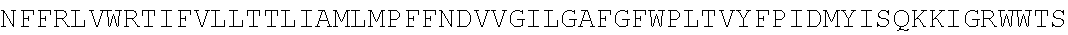

RWWLGLQLLSASCLIISLLAAVGSMAGVVLDLKTYKPFKTSY
SEQ ID NO: 44: XP_006590854.1; XP_003540867; AAP4 [Glycine max] (genomic)
ATGTTGCCAAGAAGTAGAACCCTTCCTAGCAGAATCCACCAAGGAATTATAGAAGA
GAGGCATAATGTCAGGCACTACTTACAAGTTGAAGTGCGACCCAATAATACCCAA
ACGGAGACCGAAGCGATGAATATCCAGTCTAACTATTCCAAGTGCTTCGATGATG
ATGGTCGCTTGAAGAGAACAGGAACATTTTGGATGGCAACTGCTCATATCATCACT
GCTGTGATAGGCTCAGGAGTCCTTTCACTAGCATGGGCGGTTGCTCAGCTTGGTT
GGGTTGCTGGACCTATTGTCATGTTTCTCTTTGCCGTCGTCAATCTCTACACTTCC
AACCTATTAACACAGTGTTACAGGACCGGTGACTCCGTTACTGGACACAGAAATTA
CACCTACATGGAGGCAGTCAACTCCATCTTGGGAGGAAAAAAGGTCAAGTTATGT
GGCCTCATCCAATATATCAATCTGTTTGGAGTTGCAATTGGATACACCATTGCTGC
CTCTGTCAGTATGATGGCCATAAAAAGGTCGAATTGTTATCACAGCAGTCATGGAA
AAGATCCCTGCCACATGTCAAGCAATGGGTATATGATAACATTCGGAATAGCAGAA
GTGATATTTTCCCAAATCCCAGACTTTGATCAGGTGTGGTGGCTATCCATAGTTGC
AGCTATCATGTCCTTCACTTATTCTTCAGTTGGATTGAGTCTTGGCGTGGCCAAAG
TAGCAGAAAATAAAAGTTTCAAAGGAAGCCTGATGGGAATTAGCATTGGCACAGTA
ACACAAGCCGGAACAGTCACTAGCACACAGAAAATATGGAGGAGTTTACAAGCTC
TCGGGGCAATGGCCTTTGCATACTCCTTTTCCATTATCCTCATCGAAATTCAGGAC
ACCATAAAATCTCCTCCTGCAGAGCACAAGACCATGAGAAAGGCCACAACTTTGA
GCATCGCAGTTACTACAGTGTTCTATTTACTCTGTGGATGCATGGGTTATGCAGCC
TTCGGAGATAATGCACCTGGAAACCTCTTGACTGGTTTTGGGTTCTATAACCCTTA
CTGGCTTCTGGACATTGCCAACCTTGCAATTGTTATCCACCTAGTTGGGGCATACC
AGGTTTTTTCCCAGCCCTTATTTGCATTTGTGGAAAAATGGAGTGCACGTAAATGG
CCAAAGAGCAATTTTGTCACCGCAGAATATGATATACCCATTCCCTGCTTTGGTGT
GTACCAACTCAACTTCTTCCGCTTAGTATGGAGGACCATTTTTGTGCTGTTGACGA
CCCTCATAGCCATGCTCATGCCTTTTTTCAACGATGTGGTTGGAATACTTGGCGCT

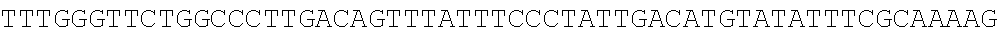

AAGATTGGACGATGGACCAGTCGCTGGCTTGGACTTCAGTTACTTAGTGCCAGTT
GCCTCATCATTTCATTGTTAGCTGCAGTTGGTTCCATGGCAGGGGTGGTTTTGGA
CCTCAAGACTTACAAGCCATTTAAAACTAGTTATTAA
SEQ ID NO: 45: RZB79331.1 AAP2 isoform B [Glycine soja] (protein)
MNIQSNYSKCFDDDGRLKRTGTFWMATAHIITAVIGSGVLSLAWAVAQLGWVAGPIV
MFLFAVVNLYTSNLLTQCYRTGDSVTGHRNYTYMEAVNSILGGKKVKLCGLIQYINLF
GVAIGYTIAASVSMMAIKRSNCYHSSHGKDPCHMSSNGYMITFGIAEVIFSQIPDFDQV
WWLSIVAAIMSFTYSSVGLSLGVAKAENKSFKGSLMGISIGTVTQAGTVTSTQKIWR
SLQALGAMAFAYSFSIILIEIQDTIKSPPAEHKTMRKATTLSIAVTTVFYLLCGCMGYAAF
GDNAPGNLLTGFGFYNPYWLLDIANLAIVIHLVGAYQVFSQPLFAFVEKWSARKWPKS

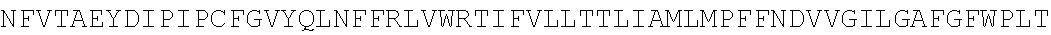

VYFPIDMYISQKKIGRWTSRWLGLQLLSASCLIISLLAAVGSMAGVVLDLKTYKPFKTSY
SEQ ID NO: 46: RZB79331.1 AAP2 isoform B [Glycine soja] (genomic)
ATGAATATCCAGTCTAACTATTCCAAGTGCTTCGATGATGATGGTCGCTTGAAGAG
AACAGGAACATTTTGGATGGCAACTGCTCATATCATCACTGCTGTGATAGGCTCAG
GAGTCCTTTCACTAGCATGGGCGGTTGCTCAGCTTGGTTGGGTTGCTGGACCTAT
TGTCATGTTTCTCTTTGCCGTCGTCAATCTCTACACTTCCAACCTATTAACACAGTG
TTACAGGACCGGTGACTCCGTTACTGGACACAGAAATTACACCTACATGGAGGCA
GTCAACTCCATCTTGGGAGGAAAAAAGGTCAAGTTATGTGGCCTCATCCAATATAT
CAATCTGTTTGGAGTTGCAATTGGATACACCATTGCTGCCTCTGTCAGTATGATGG
CCATAAAAAGGTCGAATTGTTATCACAGCAGTCATGGAAAAGATCCCTGCCACATG
TCAAGCAATGGGTATATGATAACATTCGGAATAGCAGAAGTGATATTTTCCCAAAT
CCCAGACTTTGATCAGGTGTGGTGGCTATCCATAGTTGCAGCTATCATGTCCTTCA
CTTATTCTTCAGTTGGATTGAGTCTTGGCGTGGCCAAAGTAGCAGAAAATAAAAGT
TTCAAAGGAAGCCTGATGGGAATTAGCATTGGCACAGTAACACAAGCCGGAACAG
TCACTAGCACACAGAAAATATGGAGGAGTTTACAAGCTCTCGGGGCAATGGCCTT
TGCATACTCCTTTTCCATTATCCTCATCGAAATTCAGGACACCATAAAATCTCCTCC
TGCAGAGCACAAGACCATGAGAAAGGCCACAACTTTGAGCATCGCAGTTACTACA
GTGTTCTATTTACTCTGTGGATGCATGGGTTATGCAGCCTTCGGAGATAATGCACC
TGGAAACCTCTTGACTGGTTTTGGGTTCTATAACCCTTACTGGCTTCTGGACATTG
CCAACCTTGCAATTGTTATCCACCTAGTTGGGGCATACCAGGTTTTTTCCCAGCCC
TTATTTGCATTTGTGGAAAAATGGAGTGCACGTAAATGGCCAAAGAGCAATTTTGT
CACCGCAGAATATGATATACCCATTCCCTGCTTTGGTGTGTACCAACTCAACTTCT
TCCGCTTAGTATGGAGGACCATTTTTGTGCTGTTGACGACCCTCATAGCCATGCTC

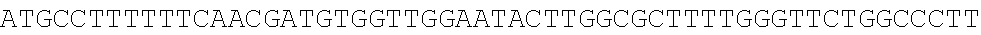

GACAGTTTATTTCCCTATTGACATGTATATTTCGCAAAAGAAGATTGGACGATGGA
CCAGTCGCTGGCTTGGACTTCAGTTACTTAGTGCCAGTTGCCTCATCATTTCATTG
TTAGCTGCAGTTGGTTCCATGGCAGGGGTGGTTTTGGACCTCAAGACTTACAAGC
CATTTAAAACTAGTTATTAA
SEQ ID NO: 47: AAK33098.1 amino acid transporter [Glycine max] (protein)
MLPRSRTLPSRIHQGIIEERHNVRHYLQVEVRPNNTQTETEAMNIQSNYSKCFDDDGR
LKRTGTFWMATAHIITAVIGSGVLSLAWAVAQLGWVAGPIVMFLFAVVNLYTSNLLTQC
YRTGDSVSGHRNYTYMEAVNSILGGKKVKLCGLTQYINLFGVAIGYTIAASVSMMAIKR
SNCYHSSHGKDPCHMSSNGYMITFGIAEVIFSQIPDFDQVWWLSIVAAIMSFTYSSVG
LSLGVAKVAENKSFKGSLMGISIGTVTQAGTVTSTQKIWRSLQALGAMAFAYSFSIILIEI
QDTIKSPPAEHKTMRKATTLSIAVTTVFYLLCGCMGYAAFGDNAPGNLLTGFGFYNPY
WLLDIANLAIVIHLVGAYQVFSQPLFAFVEKWSARKWPKSNFVTAEYDIPIPCFGVYQL

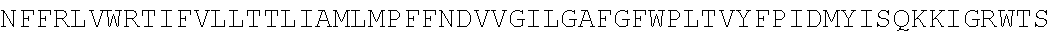

RWLGLQLLSASCLIISLLAAVGSMAGVVLDLKTYKPFKTSY
SEQ ID NO: 48: AAK33098.1 amino acid transporter [Glycine max] (genomic)
ATGTTGCCAAGAAGTAGAACCCTTCCTAGCAGAATCCACCAAGGAATTATAGAAGA
GAGGCATAATGTCAGGCACTACTTACAAGTTGAAGTGCGACCCAATAATACCCAA
ACGGAGACCGAAGCGATGAATATCCAGTCTAACTATTCCAAGTGCTTCGATGATG
ATGGTCGCTTGAAGAGAACAGGAACATTTTGGATGGCAACTGCTCATATCATCACT
GCTGTGATAGGCTCAGGAGTCCTTTCACTAGCATGGGCGGTTGCTCAGCTTGGTT
GGGTTGCTGGACCTATTGTCATGTTTCTCTTTGCCGTCGTCAATCTCTACACTTCC
AACCTATTAACACAGTGTTACAGGACCGGTGACTCCGTTTCTGGACACAGAAATTA
CACCTACATGGAGGCAGTCAACTCCATCTTGGGAGGAAAAAAGGTCAAGTTATGT
GGCCTCACCCAATATATCAATCTGTTTGGAGTTGCAATTGGATACACCATTGCTGC
CTCTGTCAGTATGATGGCCATAAAAAGGTCGAATTGTTATCACAGCAGTCATGGAA
AAGATCCCTGCCACATGTCAAGCAATGGGTATATGATAACATTCGGAATAGCAGAA
GTGATATTTTCCCAAATCCCAGACTTTGATCAGGTGTGGTGGCTATCCATAGTTGC
AGCTATCATGTCCTTCACTTATTCTTCAGTTGGATTGAGTCTTGGCGTGGCCAAAG
TAGCAGAAAATAAAAGTTTCAAAGGAAGCCTGATGGGAATTAGCATTGGCACAGTA
ACACAAGCCGGAACAGTCACTAGCACACAGAAAATATGGAGGAGTTTACAAGCTC
TCGGGGCAATGGCCTTTGCATACTCCTTTTCCATTATCCTCATCGAAATTCAGGACC
ACCATAAAATCTCCTCCTGCAGAGCACAAGACCATGAGAAAGGCCACAACTTTGA
GCATCGCAGTTACTACAGTGTTCTATTTACTCTGTGGATGCATGGGTTATGCAGCC
TTCGGAGATAATGCACCTGGAAACCTCTTGACTGGTTTTGGGTTCTATAACCCTTA
CTGGCTTCTGGACATTGCCAACCTTGCAATTGTTATCCACCTAGTTGGGGCATACC
AGGTTTTTTCCCAGCCCTTATTTGCATTTGTGGAAAAATGGAGTGCACGTAAATGG
CCAAAGAGCAATTTTGTCACCGCAGAATATGATATACCCATTCCCTGCTTTGGTGT
GTACCAACTCAACTTCTTCCGCTTAGTATGGAGGACCATTTTTGTGCTGTTGACGA
CCCTCATAGCCATGCTCATGCCTTTTTTCAACGATGTGGTTGGAATACTTGGCGCT

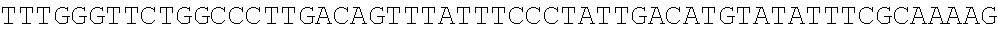

AAGATTGGACGATGGACCAGTCGCTGGCTTGGACTTCAGTTACTTAGTGCCAGTT
GCCTCATCATTTCATTGTTAGCTGCAGTTGGTTCCATGGCAGGGGTGGTTTTGGA
CCTCAAGACTTACAAGCCATTTAAAACTAGTTATTAA
SEQ ID NO: 49: XP_003542145.1; XP_006596210; XP_003522571;
XP_003527948; AAP3 [Glycine max] (protein)
MMENGGKQTFEVSNDTLQRVGSKSFDDDGRLKRTGTIWTASAHIITAVIGSGVLSLA
WAIAQLGWIAGPVVMILFSIVTYYTSTLLATCYRSGDQLSGKRNYTYTQAVRSYLGGF
SVKCGWVQYANLFGVAIGYTIAASISMMAIKRSNCYHSSGGKNPCKMNSNWYMISY
GVSEIIFSQIPDFHELWWLSIVAAVMSFTYSFIGLGLGIGKVIGNGRIKGSLTGVTIGVT
ESQKIWRTFQALGNIAFAYSYSMILIEIQDTIKSPPAESETMSKATLISVLVTTVFYMLCG
CFGYASFGDASPGNLLTGFGFYNPFWLIDIANAGIVIHLVGAYQVYCQPLFSFVESNAA

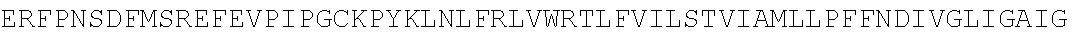

FWPLTVYLPVEMYITQTKIPKWGIKWIGLQMLSVACFVITILAAAGSIAGVIDDLKVYKPF
VTSY
SEQ ID NO: 50: XP_003542145.1; XP_006596210; XP_003522571;
XP_003527948; APP3 [Glycine max] (genomic)
ATGATGGAAAACGGTGGCAAACAGACATTTGAAGTCTCCAATGACACGCTTCAAC
GAGTAGGTTCCAAGAGCTTTGATGATGATGGCCGTCTCAAAAGAACTGGAACTAT
TTGGACTGCAAGTGCCCACATAATAACAGCTGTTATTGGTTCTGGGGTGCTATCTT
TGGCTTGGGCTATTGCTCAGCTAGGTTGGATTGCTGGTCCTGTGGTGATGATTCT
ATTCTCTATTGTGACTTATTATACCTCAACTCTTCTAGCTACTTGTTACCGTTCTGG
TGACCAACTCAGTGGCAAGAGAAACTACACTTACACACAAGCTGTTAGATCCTACC
TTGGCGGTTTTTCGGTCAAGTTTTGTGGGTGGGTTCAGTATGCGAACCTTTTTGGA
GTGGCAATTGGGTACACCATAGCAGCTTCCATAAGCATGATGGCAATCAAAAGGT
CTAATTGTTATCATAGTAGCGGGGGGAAAAATCCATGCAAAATGAACAGCAATTGG
TACATGATTTCATATGGTGTTTCGGAAATTATCTTCTCCCAAATTCCAGATTTCCAT
GAGTTGTGGTGGCTCTCTATTGTAGCTGCTGTCATGTCCTTCACATACTCATTCAT
TGGACTTGGCCTTGGTATTGGTAAAGTTATAGGAAACGGAAGAATTAAAGGAAGC
CTAACTGGTGTAACTATTGGGACTGTGACAGAATCCCAAAAAATTTGGAGAACTTT
CCAAGCGCTTGGAAACATAGCCTTTGCTTACTCCTACTCAATGATCCTTATTGAAA
TTCAGGACACAATCAAATCCCCTCCAGCAGAGTCAGAGACAATGTCCAAGGCTAC
TTTAATAAGTGTTTTGGTCACAACCGTTTTCTATATGCTATGTGGTTGCTTTGGCTA
TGCTTCTTTTGGAGATGCAAGTCCGGGAAACCTTCTCACTGGCTTTGGCTTCTATA
ACCCATTTTGGCTCATTGACATAGCCAATGCTGGCATTGTTATCCACCTTGTTGGT
GCATACCAAGTTTACTGCCAACCCCTCTTCTCATTCGTCGAATCAAATGCGGCAGA
AAGGTTCCCTAATAGTGATTTTATGAGCAGAGAGTTTGAAGTACCAATCCCTGGTT
GCAAACCCTACAAGCTCAACCTCTTCAGGTTGGTTTGGAGGACACTTTTTGTGATT
TTGTCAACTGTGATAGCCATGCTCCTACCATTCTTCAATGACATTGTAGGGCTTAT

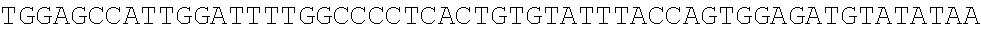

CTCAAACTAAGATACCAAAGTGGGGCATAAAATGGATAGGCCTACAAATGCTTAGT
GTTGCATGCTTTGTAATTACTATATTAGCTGCAGCAGGTTCCATTGCTGGGGTTAT
TGATGATCTTAAAGTTTACAAGCCATTTGTTACCAGCTACTAA
SEQ ID NO: 51: KHN19623.1; KHN44307; AAP 3 [Glycine soja] (protein)
MENGGKQTFEVSNDTLQQGGSKSFDDDGRLKRTGTIWTASAHIVTAVIGSGVLSLAW
AIAQLGWLAGPIVMILFSIVTYYTSTLLACCYRSGDQLSGKRNYTYTQAVRSNLGGLAV
MFCGWVQYANLFGVAIGYTIAASISMMAVKRSNCYHSSGGKNPCKMNSNWYMISYG
VAEIIFSQIPDFHELWWLSIVAAVMSFTYSFIGLGLGIGKVIGNGRIKGSLTGVTVGTVTE
SQKIWRSFQALGNIAFAYSYSMILIEIQDTIKSPPAESQTMSKATLISVLITTVFYMLCGC
FGYASFGDASPGNLLTGFGFYNPYWLIDIANVGIVIHLVGAYQVYCQPLFSFVESHAAA

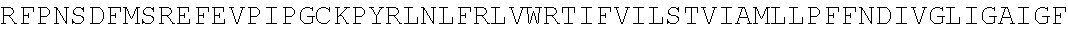

WPLTVYLPVEMYITQTKIPKWGPRWICLQMLSAACFVVTLLAAAGSIAGVIDDLKVYKP
FVTSY
SEQ ID NO: 52: KHN19623.1; KHN44307; AAP 3 [Glycine soja] (genomic)
ATGGAAAACGGTGGCAAACAGACATTTGAAGTCTCAAATGACACGCTTCAACAAG
GAGGTTCCAAGAGCTTTGATGATGATGGCCGTCTCAAAAGAACTGGAACTATATG
GACTGCAAGTGCCCACATAGTAACAGCTGTTATTGGTTCTGGGGTGCTATCTTTG
GCTTGGGCGATTGCTCAGCTAGGTTGGCTTGCTGGTCCTATTGTGATGATTCTGT
TCTCTATTGTGACTTATTATACCTCAACTCTTCTAGCTTGTTGTTACCGTTCTGGTG
ACCAACTCAGTGGCAAGAGAAACTACACTTACACACAAGCTGTTAGATCCAACCTT
GGTGGTCTTGCGGTCATGTTTTGTGGGTGGGTTCAGTATGCAAACCTATTTGGAG
TGGCAATTGGGTACACCATAGCAGCTTCCATAAGCATGATGGCAGTCAAAAGGTC
TAATTGTTATCATAGTAGCGGAGGGAAAAAATCCATGCAAAATGAATAGCAATTGGT
ACATGATTTCATATGGTGTTGCGGAAATTATCTTCTCCCAAATTCCAGATTTCCATG
AGTTGTGGTGGCTCTCTATTGTAGCTGCTGTCATGTCCTTCACATACTCATTCATT
GGACTTGGCCTTGGTATTGGTAAAGTTATAGGAAACGGAAGAATTAAAGGAAGCC
TAACTGGTGTAACTGTTGGGACTGGTGACAGAATCCCAGAAAATTTGGAGGAGTTT
CCAAGCTCTTGGTAACATAGCCTTTGCCTACTCCTACTCAATGATCCTTATTGAAAT
TCAGGACACAATCAAATCTCCTCCAGCAGAGTCACAGACAATGTCCAAGGCTACT
TTAATCAGTGTTTTGATCACAACCGTTTTCTATATGTTATGTGGCTGCTTTGGCTAT
GCTTCTTTCGGAGATGCAAGCCCGGGAAACCTTCTCACTGGCTTCGGCTTCTATA
ACCCATATTGGCTCATTGACATAGCCAATGTTGGCATAGTTATCCACCTTGTTGGT
GCATACCAAGTTTACTGCCAACCCCTCTTCTCATTCGTGGAATCACATGCAGCAGC
AAGGTTCCCAAATAGTGATTTTATGAGCAGAGAGTTTGAAGTACCAATCCCTGGCT
GCAAACCCTACAGGCTCAACCTCTTCAGGTTGGTTTGGAGGACAATTTTTGTGATT
TTGTCAACTGTGATAGCCATGCTCCTACCATTCTTCAATGACATTGTAGGGCTTAT

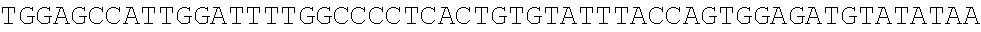

CTCAAACTAAGATACCAAAGTGGGGCCCAAGATGGATATGCCTACAAATGCTTAG
TGCTGCATGCTTTGTAGTTACTCTATTAGCTGCAGCAGGTTCCATTGCTGGGGTTA
TTGATGATCTTAAAGTTTACAAGCCATTCGTCACCAGCTACTAA
SEQ ID NO: 53: RZC18207.1 AAP3 isoform D [Glycine soja] (protein)
MMCLRCTGTVWTASAHIITAVIGSGVLSLAWAIAQLGWIAGPIVMVLFSAITYYTSTLLS
DCYRTGDPVTGKRNYTYMDAIQSNFGGNGFKVKLCGLVQYINLFGVAIGYTIAASTSM
MAIERSNCYHKSGGKDPCHMNSNMYMISFGIVEIIFSQIPGFDQLWWLSIVAAVMSFT
YSTIGLGLGIGKVIENRGVGGSLTGITIGTVTQTEKVWRTMQALGDIAFAYSYSLILVEIQ
DTVKSPPSESKTMKKASFISVAVTSIFYMLCGCFGYAAFGDASPGNLLTGFGFYNPYW
LLDIANAAIVIHLVGSYQVYCQPLFAFVEKHAARMLPDSDFVNKEIEIPIPGFHSYKVNLF

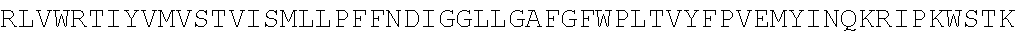

WICLQILSMACLLMTIGAAAGSIAGIAIDLQTYKPFKTNY
SEQ ID NO: 54: RZC18207.1 APP3 isoform D [Glycine soja] (genomic)
ATGATGTGTTTGAGATGTACAGGGACGGTGTGGACTGCAAGTGCACACATAATA
CTGCAGTGATTGGGTCTGGGGTGCTGTCTCTGGCTTGGGCTATAGCTCAGCTTGG
ATGGATTGCTGGTCCTATTGTCATGGTTCTCTTTTCTGCCATCACTTACTACACTTC
CACTCTTCTCTCTGATTGTTATCGTACTGGTGATCCTGTAACTGGCAAGAGAACT
ACACTTACATGGACGCTATTCAGTCTAACTTTGGTGGAAATGGCTTTAAGGTCAAG
CTGTGTGGGCTAGTTCAGTACATTAACCTTTTCGGAGTCGCCATTGGTTACACTAT
AGCGGCTTCCACTAGCATGATGGCAATTGAAAGATCTAATTGTTACCACAAGAGTG
GAGGGAAAGATCCATGTCATATGAACAGTAACATGTACATGATTTCATTTGGTATA
GTGGAAATTATTTTCTCACAAATTCCGGGCTTCGATCAATTGTGGTGGCTCTCCAT
TGTAGCTGCTGTCATGTCCTTCACATACTCCACTATTGGGCTAGGCCTTGGTATTG
GAAAAGTTATTGAAAATAGAGGAGTCGGGGGAAGCCTAACCGGGATAACAATTGG
TACCGTGACACAAACTGAAAAAGTTTGGAGAACCATGCAAGCTCTTGGTGACATA
GCCTTTGCCTATTCATACTCCCTCATCCTTGTAGAAATTCAGGACACAGTGAAATC
CCCTCCATCAGAGTCAAAAACAATGAAGAAGGCTAGTTTCATCAGTGTTGCAGTAA
CCAGCATTTTCTACATGCTTTGTGGTTGCTTTGGTTATGCTGCTTTTGGAGATGCA
AGCCCTGGAAACCTTCTCACTGGCTTTGGTTTCTACAACCCATATTGGCTCCTTGA
CATAGCTAATGCTGCCATAGTGATCCACCTTGTTGGTTCATACCAAGTTTACTGCC
AGCCCCTCTTCGCCTTCGTTGAGAAACACGCGGCGCGTATGCTCCCAGATAGTGA
TTTTGTGAACAAAGAAATTGAAATTCCAATCCCTGGTTTCCATTCCTACAAGGTCAA
CCTTCTTCAGATTGGTTTGGAGGACAATATATGTAATGGTGAGCACTGTAATATCAA

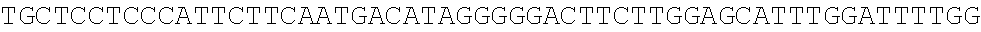

CCCCTTACTGTGTATTTCCCAGTGGAGATGTACATTAATCAAAAGAGAATACCAAA
ATGGAGCACAAAGTGGATCTGCCTCCAAATACTTAGCATGGCTTGCCTTTTGATGA
CTATAGGAGCTGCAGCTGGCTCTATTGCTGGGATTGCCATTGATCTTCAAACTTAC
AAGCCTTTCAAAACCAACTATTGA
SEQ ID NO: 55: RZC13226.1 Amino acid permease 2 isoform B, partial
[Glycine soja] (protein)
KFALFLRVFCVWKFSFHQIKMPENAATTNLNHLQVFGIEDDVPSHSQNNSKCYDDDG
RLKRTGNVWTASSHIITAVIGSGVLSLAWAIAQLGWIAGPTVMFLFSLVTFYTSSLLAD
CYRAGDPNSGKRNYTYMDAVRSILGGANVTLCGIFQYLNLLGIVGYTIAASISMMAIKR
SNCFHKSGGKNPCHMSSNVYMIIFGATEIFLSQIPDFDQLWWLSTVAAIMSFTYSIIGLS
LGIAKVAETGTFKGGLTGISIGPVSETQKIWRTSQALGDIAFAYSYAVVLIEIQDTIKSPP
SEAKTMKKATLISIAVTTTFYMLCGCMGYAAFGDAAPGNLLTGFGFYNPYWLIDIANAA
IVIHLVGAYQVFSQPIFAFVEKEVTQRWPHIEREFKIPIPGFSPYKLKVFRLVLRTVFVVL

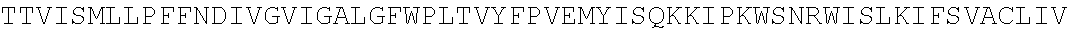

SVVAAVGSVAGVLLDLKKYKPFHSHY
SEQ ID NO: 56: RZC13226.1 Amino acid permease
2 isoform B, partial [Glycine soja]
(genomic): >Glyma.05G194600 | Chr05:37909533..37914347 reverse

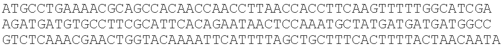

ATACACGACTCTGTTTCATATAAACTAGAATTTAGTTAGAATGAATCGACACGCTGA
TAAACAATTTTTTATTATGTTATCTAATTATAAAAATTTTGTACAATAAAATTATTAAA
TTTTATAATAATAAAATTTATATTTGGTATATTTATTGACCGTGTAAACTGGTTTATAG
AGATTTTATAACAACTCTTAAGTTTTAGTCTATTGACTTTAATAATAATTATCTTAAA
ATTTATATTTAACTTTATTTTTAAATAATTTTTTAATATTATCAATGTATCCATAAAAAT
GAAACTATAAAATTTAACTTTGTAATATAACTGTTCCACTATTTGAAAATCAAACATC
AGACATTGTTAATTATTGTTTCTATCTCCATTTTTTTTTAAAATCTTTTTGCCCCACAC
ACATCTCAAAATTCCTTCAAAAATCCAAGGAAATAACAAAAGCTGACCCTTTTTTTA
TGTACAAAAAGTATTTTTCATAATGTAAATTAATATATAATGTACATGTGATGATATG

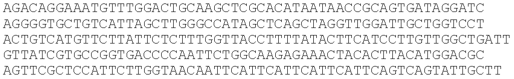

TTTTTATATTCTTTTCACAAATTAATGATATGCATGCATGTTGTTGACCTTAAATGAT
ACTATATCATATAGTACATATAGTATATATTGTTGGAAAGTCTCACATCATATTATCT
ATTTTAATTTTTGAGATGTAATTTATATATTTGTTGAGTAATTTTATAATATCAATAAG
ATGTATGTAATAAATTAAACATTAATAACTTATAATTAATGGGATTGTCATGATCATA
CTGATGATAAGGTCATTATCACCTCATGAAAATATATGAGATTATGTCCGCCAAAG
AAGATGCTACTTGTCAAAAGGGATTCAACTCATGCACATGCTCTTGTTCACATTCT
ACTAGCTAGTTATCTAAAACTTAATATAACATGCACTTTAGGGTTGGCGAATTTAAT
ATAACTCATGCACATGGTCTTTGTCTTTTATTACCAAAAAAAGAAAGGGAGTTTTCC
CTAGAAAATACGATTTAATTTTTAATTGCCAATTTTGTATTCTGTAGTCTTAATATAT
ATAAAATTGTCTGGCCAATGCCTCGTGTTGGCTGCACAGTGAATAGCAATATTTTT
TATTTTTTCTCTTTTATTCTCACTTTCTCTTTTTGATTTTTAAAAAACCCTAGACTCTA
GTATTATGATAAATCTCCTCACTAACGGTATGCCACGTGCCTGTCAAAGAAAAATA
GTATGCCACGCGTATTAATTTCCCACTCTAACATTAATCATTAATATGTGCGCAAAC
CAAGAGCATCTTAAATTGATGATTTATTTGTTTGTTACACTTACAAGAACAGGTTAC
AGTATCCAGCTTGCTTTACTGAGAGGGTTTTGCTAATTAGTGTTTTTAGATATTGGT
TAACGGAGTTAAAAAAATATATTTATTATAAAATTATAGACAAATATAAAAAAGTCA
TAAAAAATATAATTTTATATATTTTAACAAAAAAATTAGTTTCTCAATCAATACTTCA
AAAATATTGATTAACATTTACCTTATTTCAATACTTCTCATTCAACCATATATATACA
TGGGATAATATTAAATTAAAAAAATATTTATTATAAAAATTATATATAAATATAAAAA
AAGTCATAAACAACACAATTTTGCATGTATTAGCAAAAATAATAATTTCTTAATCAAT
GCTTCAAAATATTAGTTAACATTTACCTTATTTCAATACTTCTCATTCAACCATATAT
ATACATGGGATAATATTAAATTAATTTAACTTTTTTAACTAGTTTGGCTAAATCAAGC
TTATTTAATGTCTATGATAGTACTTTGGAAATTGGAAGATGCATGTATAATAATTTTA
ACGTATCAATAACCAAATTAAAAATTAGGTCAAAAGTTTGAGAAGGCAGAAAGAGC
ATACAAATTTCATATGATTTATAAATAATAGCTTAGTTCAGATAAACTTATTAATAAT
TTTAACATATGATTTTATCTTATATTTCATATTGTACAATGCAATATAATGTATTTTGC

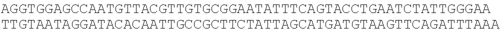

CTTCATAATGAATGATATTACTTGTAGTCTACCAACATCATTTCATGTCAGTGTTAT
TTCTTATCATTTAATGATAATTGAGCCTATCTCATTTTCCACACACTTTTATTTTGAT
TTGGATCAAAATTAGTTACCTAAAAACTACACAAATACTTAAAAAAAAATGTTCACG
AGTTAACTATGAGTAAAATATCTAATGATTGTATCACACACAAGTGACACAACCTGA
AATTAATTTCCTTGAACTGATTGATAGGGATAGTATTATACACTACAGTTTGATGAT

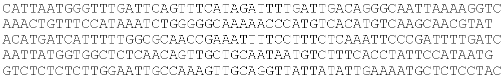

ACTTTTAGTTTTTTTTTAATACAACACTTTTCTTAGGGTTACATTGCATCATATAGGCT

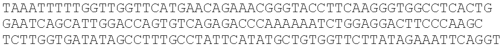

ATCTCCTTCAAAAACTAAGAATTAGTTAATCTAAAAAGGTTAAAATGTTCATTTGAT
TTTTATAGTTGTTCTCATTTTAAAAGTTTCGTCCATGTTCAGAAGGAAAAAAAAAACA
ATAGCTTTTAGTCCCTACACAATTTTTTGTGGCAGTTTATCACTACTTTTTGTAACT
GTCAAAAATAAGTTTTGATATTCTCAGCAAGTACCAAAACTTAACTTAAACACATTG
TAGGGACTAAAACTTATTATTTTTCGTATAAAAACTAAAACTTAAAATGAAGAGAA
CTGTAGAGATCAAATGAGTAGTTTAACATTAAGAAAAGAAGAATATGATTTATTACA

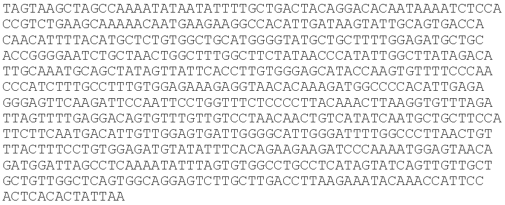

SEQ ID NO: 57: XP_003524313.1 AAP 4-like [Glycine max] (protein)

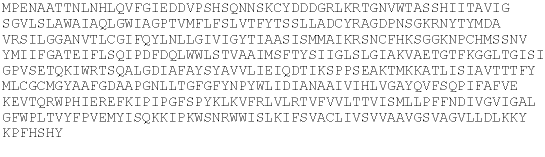

SEQ ID NO: 58: XP_003524313.1 AAP 4-like [Glycine max] (genomic)
ATGCCTGAAAACGCAGCCACAACCAACCTTAACCACCTTCAAGTTTTTGGCATCGA
AGATGATGTGCCTTCGCATTCACAGAATAACTCCAAATGCTATGATGATGATGGCC
GTCTCAAACGAACTGGAAATGTTTGGACTGCAAGCTCGCACATAATAACCGCAGT
GATAGGATCAGGGGTGCTGTCATTAGCTTGGGCCATAGCTCAGCTAGGTTGGATT
GCTGGTCCTACTGTCATGTTCTTATTCTCTTTGGTTACCTTTTATACTTCATCCTTG
TTGGCTGATTGTTATCGTGCCGGTGACCCCAATTCTGGCAAGAGAAACTACACTTA
CATGGACGCAGTTCGCTCCATTCTTGGTGGAGCCAATGTTACGTTGTGCGGAATA
TTTCAGTACCTGAATCTATTGGGAATTGTAATAGGATACACAATTGCCGCTTCTATT
AGCATGATGGCAATTAAAAGGTCAAACTGTTTCCATAAATCTGGGGGCAAAAACCC
ATGTCACATGTCAAGCAACGTATACATGATCATTTTTGGCGCAACCGAAATTTTCC
TTTCTCAAATTCCCGATTTTGATCAATTATGGTGGCTCTCAACAGTTGCTGCAATAA
TGTCTTTCACCTATTCCATAATTGGTCTCTCTCTTGGAATTGCCAAAGTTGCAGAAA
CGGGTACCTTCAAGGGTGGCCTCACTGGAATCAGCATTGGACCAGTGTCAGAGA
CCCAAAAAATCTGGAGGACTTCCCAAGCTCTTGGTGATATAGCCTTTGCCTATTCA
TATGCTGTGGTTCTTATAGAAATTCAGGACACAATAAAATCTCCACCGTCTGAAGC
AAAAACAATGAAGAAGGCCACATTGATAAGTATTGCAGTGACCACAACATTTTACA
TGCTCTGTGGCTGCATGGGGTATGCTGCTTTTGGAGATGCTGCACCGGGGAATCT
GCTAACTGGCTTTGGCTTCTATAACCCATATTGGCTTATAGACATTGCAAATGCAG
CTATAGTTATTCACCTTGTGGGAGCATACCAAGTGTTTTCCCAACCCATCTTTGCC
TTTGTGGAGAAAGAGGTAACACAAAGATGGCCCCACATTGAGAGGGAGTTCAAGA
TTCCAATTCCTGGTTTCTCCCCTTACAAACTTAAGGTGTTTAGATTAGTTTTGAGGA
CAGTGTTTGTTGTCCTAACAACTGTCATATCAATGCTGCTTCCATTCTTCAATGACA

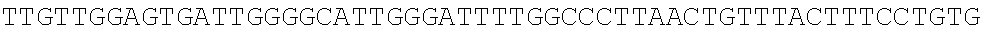

GAGATGTATATTTCACAGAAGAAGATCCCAAAATGGAGTAACAGATGGATTAGCCT
CAAAATATTTAGTGTGGCCTGCCTCATAGTATCAGTTGTTGCTGCTGTTGGCTCAG
TGGCAGGAGTCTTGCTTGACCTTAAGAAATACAAACCATTCCACTCACACTATTAA
SEQ ID NO: 59: XP_006581782.1 AAP3 isoform X1 [Glycine max] (protein)
MVEKSSRTNLSHHQDFGMEPYSIDGVSSQTNSKFYDDDGHVKRTGTVWTTSSSHIITA
VVGSGVLSLAWAMAQMGWVAGPAVMIFFSVVTLYTTSLLADCYRCGDPVTGKRNYT
FMDAVQSILGGYYDAFCGVVQYSNLYGTAVGYTIAASISMMAIKRSNCFHSSGGKSPC
QVSSNPYMIGFGIIQILFSQIPDFHETWWLSIVAAIMSFVYSTIGLALGIAKVAEMGTFKG
SLTGVRIGTVTEATKVWGVFQGLGDIAFAYSYSQILIEIQDTIKSPPSEAKTMKKSAKISI
GVTTTFYMLCGFMGYAAFGDSAPGNLLTGFGFFNPYWLIDIANAAVIHLVGAYQVYAQ
PLFAFVEKWASKRWPEVETEYKIPIPGFSPYNLSPFRLVWRTVFVIITTFVAMLIPFFND

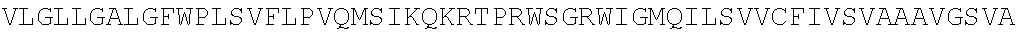

SIVLDLQKYKPFHVDY
SEQ ID NO: 60: XP_006581782.1 AAP3 isoform X1 [Glycine max] (genomic)
ATGGTTGAAAAATCTTCCAGAACCAATCTTAGCCACCATCAAGACTTTGGCATGGA
GCCTTACTCCATTGATGGTGTTTCTTCACAAACTAACTCCAAATTCTACGATGATGA
TGGCCATGTTAAACGAACAGGGACCGTTTGGACAACAAGCTCGCACATAATAACA
GCAGTGGTGGGTTCTGGGGTGCTGTCTTTGGCATGGGCCATGGCTCAAATGGGT
TGGGTTGCTGGGCCTGCAGTTATGATCTTCTTCAGTGTTGTTACGTTGTATACGAC
GTCGCTTCTGGCTGATTGTTATCGCTGTGGTGACCCTGTTACCGGGAAGAGAAAC
TATACTTTCATGGATGCAGTTCAATCCATTCTCGGTGGGTATTATGATGCATTTTGT
GGGGTAGTTCAGTACTCAAATCTTTACGGAACCGCCGTAGGATACACAATTGCAG
CTTCTATTAGCATGATGGCAATAAAAAGGTCCAACTGTTTCCATTCTTCAGGCGGA
AAAAGTCCATGTCAGGTTTCAAGCAACCCATACATGATCGGTTTTGGCATAATCCA
AATTTTATTTTCTCAAATTCCAGATTTTCATGAAACATGGTGGCTCTCCATAGTTGC
AGCAATCATGTCTTTTGTCTATTCCACAATTGGGCTCGCTCTTGGCATTGCCAAAG
TTGCAGAAATGGGTACTTTCAAGGGTAGTCTCACAGGAGTAAGGATTGGAACTGT
GACCGAGGCCACAAAAGTATGGGGGGTTTTCCAAGGTCTTGGTGACATAGCCTTC
GCCTATTCATATTCTCAAATTCTCATTGAAATTCAGGACACCATAAAATCTCCACCA
TCGGAAGCAAAGACAATGAAGAAGTCTGCTAAGATAAGTATTGGAGTAACCACAA
CATTTTATATGCTTTGTGGTTTCATGGGCTATGCTGCTTTTGGAGATTCAGCACCT
GGGAACCTGCTCACAGGATTTGGTTTTTTTAACCCATATTGGCTCATAGATATTGC
TAATGCTGCTATCGTAATTCACCTTGTGGGAGCATACCAAGTTTATGCCCAGCCCC
TCTTTGCCTTTGTCGAGAAATGGGCTTCAAAAAGATGGCCTGAAGTTGAGACGGA
ATATAAAATTCCAATTCCTGGTTTTTCACCCTACAATCTAAGCCCATTTAGATTAGT
TTGGAGAACAGTGTTTGTTATCATAACCACTTTTGTAGCAATGTTGATTCCATTCTT

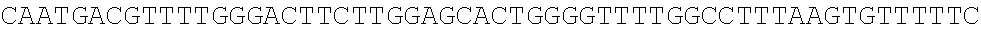

TCCCAGTGCAGATGAGTATCAAACAAAAGAGGACCCCAAGGTGGAGTGGTAGATG
GATTGGTATGCAAATCTTAAGTGTTGTTTGTTTCATAGTATCAGTTGCGGCTGCTG
TTGGCTCAGTTGCCAGTATCGTGCTTGACCTACAGAAATACAAACCGTTTCATGTA
GACTATTAA
SEQ ID NO: 61: XP_006581783.1 AAP3 isoform X2 [Glycine max] (protein)
MEPYSIDGVSSQTNSKFYDDDGHVKRTGTVWTTSSHIITAVVGSGVLSLAWAMAQMG
WVAGPAVMIFFSVVTLYTTSLLADCYRCGDPVTGKRNYTFMDAVQSILGGYYDAFCG
VVQYSNLYGTAVGYTIAASISMMAIKRSNCFHSSGGKSPCQVSSNPYYMIGFGIIQILFS
QIPDFHETWWLSIVAAIMSFVYSTIGLALGIAKVAEMGTFKGSLTGVRIGTVTEATKVW
GVFQGLGDIAFAYSYSQILIEIQDTIKSPPSEAKTMKKSAKISIGVTTTFYMLCGFMGYA
AFGDSAPGNLLTGFGFFNPYWLIDIANAAIVIHLVGAYQVYAQPLFAFVEKWASKRWP

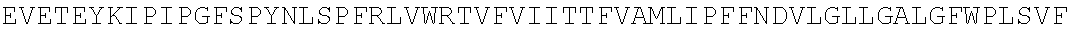

LPVQMSIKQKRTPRWSGRWIGMQILSVVCFIVSVAAAVGSVASIVLDLQKYKPFHVDY
SEQ ID NO: 62: XP_006581783.1 AAP3 isoform X2 [Glycine max] (genomic)
ATGGAGCCTTACTCCATTGATGGTGTTTCTTCACAAACTAACTCCAAATTCTACGAT
GATGATGGCCATGTTAAACGAACAGGGACCGTTTGGACAACAAGCTCGCACATAA
TAACAGCAGTGGTGGGTTCTGGGGTGCTGTCTTTGGCATGGGCCATGGCTCAAAT
GGGTTGGGTTGCTGGGCCTGCAGTTATGATCTTCTTCAGTGTTGTTACGTTGTATA
CGACGTCGCTTCTGGCTGATTGTTATCGCTGTGGTGACCCTGTTACCGGGAAGAG
AAACTATACTTTCATGGATGCAGTTCAATCCATTCTCGGTGGGTATTATGATGCATT
TTGTGGGGTAGTTCAGTACTCAAATCTTTACGGAACCGCCGTAGGATACACAATTG
CAGCTTCTATTAGCATGATGGCAATAAAAAGGTCCAACTGTTTCCATTCTTCAGGC
GGAAAAAGTCCATGTCAGGTTTCAAGCAACCCATACATGATCGGTTTTGGCATAAT
CCAAATTTTATTTTCTCAAATTCCAGATTTTCATGAAACATGGTGGCTCTCCATAGT
TGCAGCAATCATGTCTTTTGTCTATTCCACAATTGGGCTCGCTCTTGGCATTGCCA
AAGTTGCAGAAATGGGTACTTTCAAGGGTAGTCTCACAGGAGTAAGGATTGGAAC
TGTGACCGAGGCCACAAAAGTATGGGGGGTTTTCCAAGGTCTTGGTGACATAGCC
TTCGCCTATTCATATTCTCAAATTCTCATTGAAATTCAGGACACCATAAAATCTCCA
CCATCGGAAGCAAAGACAATGAAGAAGTCTGCTAAGATAAGTATTGGAGTAACCA
CAACATTTTATATGCTTTGTGGTTTCATGGGCTATGCTGCTTTTGGAGATTCAGCA
CCTGGGAACCTGCTCACAGGATTTGGTTTTTTTAACCCATATTGGCTCATAGATAT
TGCTAATGCTGCTATCGTAATTCACCTTGTGGGAGCATACCAAGTTTATGCCCAGC
CCCTCTTTGCCTTTGTCGAGAAATGGGCTTCAAAAAGATGGCCTGAAGTTGAGAC
GGAATATAAAATTCCAATTCCTGGTTTTTCACCCTACAATCTAAGCCCATTTAGATT
AGTTTGGAGAACAGTGTTTGTTATCATAACCACTTTTGTAGCAATGTTGATTCCATT

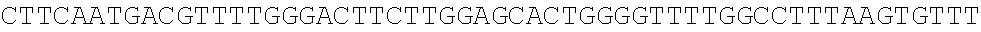

TTCTCCCAGTGCAGATGAGTATCAAACAAAAGAGGACCCCAAGGTGGAGTGGTAG
ATGGATTGGTATGCAAATCTTAAGTGTTGTTTGTTTCATAGTATCAGTTGCGGCTG
CTGTTGGCTCAGTTGCCAGTATCGTGCTTGACCTACAGAAATACAAACCGTTTCAT
GTAGACTATTAA
MAIZE
SEQ ID NO: 63: NP_001136620 uncharacterized protein LOC100216745 [Zea mays]
AQL04004.1: (protein)
MVSERQQAAGKVAAFNLTEAGFGDGSDLLDDDGRERRTGTVTASAHIITAVIGSSVL
SLAWAIAQLGWVIGPVVLLAFSAITWFCSSLLADCYRAPPGPGQGKRNYTYGQAVRS
YLGESKYRLCSLAQYVNLVGVTIGYTITTAISMGAIKRSNCFHSRGHGSDCEASNTTN
MIIFAGIQILLSQLPNFHKWWSIVAAVMSAYSSIGLLGSIAKIAGGVHVKTSTGAAV
GVDVTAAEKVWKTFQSLGDIAFAYTYSNVLIEIQDTLRSSPPENVVMKKASFIGVSTTT
AFYMLCGVLGYAAFGSDAPGNFLTGFGFYDPFWLIDVGNVCIAVHLVGAYQVFCQPIY
QFVEAWARGRWPDCAFLHAELAVVAGSSFTASPFRLVWRTAYVVLTALVATVFPFFN

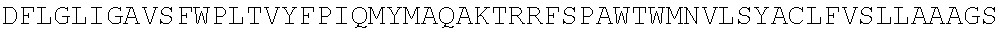

VQGLVKDLKGYKPLFKVS
SEQ ID NO: 64: NP_001136620 uncharacterized protein LOC100216745 [Zea mays]
(genomic)
ATGGTGTCGGAGAGGCAGCAGGCGGCGGGGAAGGTGGCCGCCTTCAACCTCAC
GGAGGCCGGGTTCGGCGACGGGTCGGACCTGCTGGACGACGACGGGCGCGAG
AGGCGCACGGGGACCCTGGTGACGGCGAGCGCGCACATCATCACGGCGGTGAT
CGGGTCGAGCGTGCTGTCGCTGGCGTGGGCGATCGCGCAGCTGGGGTGGGTG
ATCGGCCCCGTGGTGCTGCTGGCCTTCTCCGCCATCACCTGGTTCTGCTCCAGC
CTACTCGCCGACTGCTACCGCGCGCCGCCGGGCCCCGGCCAGGGCAAGCGGAA
CTACACCTACGGACAGGCCGTCAGGTCATACCTGGGGGAGTCCAAGTACCGGCT
GTGCTCGCTGGCGCAGTACGTGAACCTGGTGGGCGTCACCATCGGCTACACCAT
CACCACGGCCATCAGCATGGGGGCGATCAAGCGTTCCAACTGCTTCCACAGCAG
GGGCCACGGCGCCGACTGCGAGGCGTCCAACACCACCAACATGATCATCTTCGC
GGGCATCCAGATCCTGCTGTCGCAGCTCCCCAACTTCCACAAGCTCTGGTGGCTC
TCCATCGTCGCCGCCGTCATGTCCCTCGCCTACTCCTCCATCGGACTCGGCCTCT
CCATCGCAAAGATCGCAGGTGGGGTGCACGTTAAGACGTCGCTGACTGGTGCCG
CCGTGGGGGTGGACGTCACCGCGGCCGAGAAGGTCTGGAAGACGTTCCAGTCG
CTGGGGGACATCGCCTTCGCCTACACCTACTCCAACGTGCTGATCGAGATCCAG
GACACGCTGCGGTCGAGCCCGCCGGAGAACGTGGTGATGAAGAAGGCGTCCTTC
ATCGGCGTGTCCACCACCACCGCGTTCTACATGCTGTGCGGCGTGCTGGGCTAC
GCGGCGTTCGGCAGCGACGCGCCGGGCAACTTCCTCACGGGCTTCGGCTTCTAC
GACCCCTTCTGGCTCATCGACGTCGGCAACGTCTGCATCGCCGTGCACCTGGTC
GGCGCCTACCAGGTCTTCTGCCAGCCCATCTACCAGTTCGTGGAGGCCTGGGCG
CGGGGCCGCTGGCCCGACTGCGCCTTCCTCCACGCCGAGCTCGCCGTCGTCGC
CGGCTCCTCCTTCACGGCCAGCCCGTTCCGCCTCGTGTGGCGCACCGCCTACGT
CGTGCTCACCGCGCTCGTCGCCACGGTCTTCCCATTCTTCAACGACTTCCTGGGG

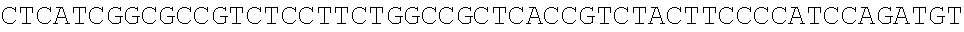

ACATGGCGCAGGCCAAGACGCGCCGCTTCTCGCCGGCGTGGACGTGGATGAAC
GTGCTCAGCTACGCTTGCCTCTTCGTCTCGCTGCTCGCCGCCGCGGGCTCAGTG
CAGGGGCTCGTCAAGGATCTCAAGGGATACAAGCCATTGTTCAAGGTCTCCTAA
SEQ ID NO: 65: PWZ15603 AAP6 [Zea mays] (protein)
MVSERQQAAGKVAAFNLTEAGFGDGSDLLDDDGRERRTGTLVTASAHIITAVIGSGVL
SLAWAIAQLGWVIGPVVLLAFSSAITWFCSSLLADCYRAPPGPGQGKRNYTYGQAVRS
YLGESKYRLCSLAQYVNLVGVTIGYTITTAISMGAIKRSNCFHSRGHGADCEASNTTN
MIIFAGIQILLSQLPNFHKLWWLSIVAAVMSLAYSSIGLGLSIAKIAGKLMHGSGVHVKTS
LTGAAVGVDVTAAEKVWKTFQSLGDIAFAYTYSNVLIEIQDTLRSSPPENVVMKKASFI
GVSTTTAFYMLCGVLGYAAFGSDAPGNFLTGFGFYDPFWLIDVGNVCIAVHLVGAYQ
VFCQPIYQFVEAWARGRWPDCAFLHAELAVVAGSSFTASPFRLVWRTAYVVLTALVA

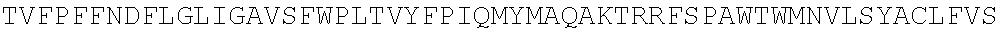

LLAAAGSVQGLVKDLKGYKPLFKVS
SEQ ID NO: 66: PWZ15603 AAP6 [Zea mays] (genomic)
ATGGTGTCGGAGAGGCAGCAGGCGGCGGGGAAGGTGGCCGCCTTCAACCTCAC
GGAGGCCGGGTTCGGCGACGGGTCGGACCTGCTGGACGACGACGGGCGCGAG
AGGCGCACGGGGACCCTGGTGACGGCGAGCGCGCACATCATCACGGCGGTGAT
CGGGTCGGGCGTGCTGTCGCTGGCGTGGGCGATCGCGCAGCTGGGGTGGGTG
ATCGGCCCCGTGGTGCTGCTGGCCTTCTCCGCCATCACCTGGTTCTGCTCCAGC
CTACTCGCCGACTGCTACCGCGCGCCGCCGGGCCCCGGCCAGGGCAAGCGGAA
CTACACCTACGGACAGGCCGTCAGGTCATACCTGGGGGAGTCCAAGTACCGGCT
GTGCTCGCTGGCGCAGTACGTGAACCTGGTGGGCGTCACCATCGGCTACACCAT
CACCACGGCCATCAGCATGGGGGCGATCAAGCGTTCCAACTGCTTCCACAGCAG
GGGCCACGGCGCCGACTGCGAGGCGTCCAACACCACCAACATGATCATCTTCGC
GGGCATCCAGATCCTGCTGTCGCAGCTCCCCAACTTCCACAAGCTCTGGTGGCTC
TCCATCGTCGCCGCCGTCATGTCCCTCGCCTACTCCTCCATCGGACTCGGCCTCT
CCATCGCAAAGATCGCAGGCAAGCTCATGCATGGCAGTGGGGTGCACGTTAAGA
CGTCGCTGACTGGTGCCGCCGTGGGGGTGGACGTCACCGCGGCCGAGAAGGTC
TGGAAGACGTTCCAGTCGCTGGGGGACATCGCCTTCGCCTACACCTACTCCAAC
GTGCTGATCGAGATCCAGGACACGCTGCGGTCGAGCCCGCCAGAGAACGTGGTG
ATGAAGAAGGCGTCCTTCATCGGCGTGTCCACCACCACCGCGTTCTACATGCTGT
GCGGCGTGCTGGGCTACGCGGCGTTCGGCAGCGACGCGCCGGGCAACTTCCTC
ACGGGCTTCGGCTTCTACGACCCCTTCTGGCTCATCGACGTCGGCAACGTCTGCA
TCGCCGTGCACCTGGTCGGCGCCTACCAGGTCTTCTGCCAGCCCATCTACCAGTT
CGTGGAGGCCTGGGCGCGGGGCCGCTGGCCCGACTGCGCCTTCCTCCACGCCG
AGCTCGCCGTCGTCGCCGGCTCCTCCTTCACGGCCAGCCCGTTCCGCCTCGTGT
GGCGCACCGCCTACGTCGTGCTCACCGCGCTCGTCGCCACGGTCTTCCCATTCT

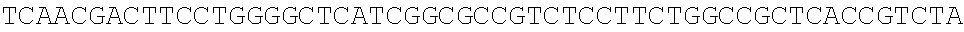

CTTCCCCATCCAGATGTACATGGCGCAGGCCAAGACGCGCCGCTTCTCGCCGGC
GTGGACGTGGATGAACGTGCTCAGCTACGCTTGCCTCTTCGTCTCGCTGCTCGC
CGCCGCGGGCTCCGTGCAGGGGCTCGTCAAGGATCTCAAGGGATACAAGCCATT
GTTCAAGGTCTCCTAA
SEQ ID NO: 67: ONM51229.1 Amino acid permease 6 [Zea mays] (protein)
MVSERQQAAGKVAAFNLTEAGFGDGSDLLDDDGRERRTGTLVTASAHIITAVIGSGVL
SLAWAIAQLGWVIGPVVLLAFSAITWFCSSLLADCYRAPPGPGQGKRNYTYGQAVRS
YLGESKYRLCSLAQYVNLVGVTIGYTITTAISMGAIKRSNCFHSRGHGADCEASNTTN
MIIFAGIQILLSQLPNFHKLWWLSIVAAVMSLAYSSIGLGLSIAKIAGKLMHGSYCGVHV
KTSLTGAAVGVDVTAAEKVWKTFQSLGDIAFAYTYSNVLIEIQDTLRSSPPENVVMKKA
SFIGVSTTTAFYMLCGVLGYAAFGSDAPGNFLTGFGFYDPFWLIDVGNVCIAVHLVGA
YQVFCQPIYQFVEAWARGRWPDCAFLHAELAVVAGSSFTASPFRLVWRTAYVVLTAL

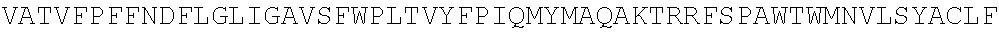

VSLLAAAGSVQGLVKDLKGYKPLFKVS
SEQ ID NO: 68: ONM51229.1 Amino acid permease 6 [Zea mays] (genomic)
ATGGTGTCGGAGAGGCAGCAGGCGGCGGGGAAGGTGGCCGCCTTCAACCTCAC
GGAGGCCGGGTTCGGCGACGGGTCGGACCTGCTGGACGACGACGGGCGCGAG
AGGCGCACGGGGACCCTGGTGACGGCGAGCGCGCACATCATCACGGCGGTGAT
CGGGTCGGGCGTGCTGTCGCTGGCGTGGGCGATCGCGCAGCTGGGGTGGGTG
ATCGGCCCCGTGGTGCTGCTGGCCTTCTCCGCCATCACCTGGTTCTGCTCCAGCT
ACTCGCCGACTGCTACCGCGCGCCGCCGGGCCCCGGCCAGGGCAAGCGGAACT
ACACCTACGGACAGGCCGTCAGGTCATACCTGGGGGAGTCCAAGTACCGGCTGT
GCTCGCTGGCGCAGTACGTGAACCTGGTGGGCGTCACCATCGGCTACACCATCA
CCACGGCCATCAGCATGGGGGCGATCAAGCGTTCCAACTGCTTCCACAGCAGGG
GCCACGGCGCCGACTGCGAGGCGTCCAACACCACCAACATGATCATCTTCGCGG
GCATCCAGATCCTGCTGTCGCAGCTCCCCAACTTCCACAAGCTCTGGTGGCTCTC
CATCGTCGCCGCCGTCATGTCCCTCGCCTACTCCTCCATCGGACTCGGCCTCTCC
ATCGCAAAGATCGCAGGCAAGCTCATGCATGGCAGCTACTGTGGGGTGCACGTT
AAGACGTCGCTGACTGGTGCCGCCGTGGGGGTGGACGTCACCGCGGCCGAGAA
GGTCTGGAAGACGTTCCAGTCGCTGGGGGACATCGCCTTCGCCTACACCTACTC
CAACGTGCTGATCGAGATCCAGGACACGCTGCGGTCGAGCCCGCCGGAGAACGT
CTGTGCGGCGTGCTGGGCTACGCGGCGTTCGGCAGCGACGCGCCGGGCAACTT
CCTCACGGGCTTCGGCTTCTACGACCCCTTCTGGCTCATCGACGTCGGCAACGTC
TGCATCGCCGTGCACCTGGTCGGCGCCTACCAGGTCTTCTGCCAGCCCATCTAC
CAGTTCGTGGAGGCCTGGGCGCGGGGCCGCTGGCCCGACTGCGCCTTCCTCCA
CGCCGAGCTCGCCGTCGTCGCCGGCTCCTCCTTCACGGCCAGCCCGTTCCGCCT
CGTGTGGCGCACCGCCTACGTCGTGCTCACCGCGCTCGTCGCCACGGTCTTCCC

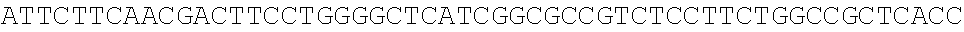

GTCTACTTCCCCATCCAGATGTACATGGCGCAGGCCAAGACGCGCCGCTTCTCG
CCGGCGTGGACGTGGATGAACGTGCTCAGCTACGCTTGCCTCTTCGTCTCGCT
GCTCGCCGCCGCGGGCTCAGTGCAGGGGCTCGTCAAGGATCTCAAGGGATACAA
GCCATTGTTCAAGGTCTCCTAA
SEQ ID NO: 69: NP_001349744.1 uncharacterized protein
LOC100501686 [Zea mays](protein)
MTQQDVEMAARHGTGADGAGFYPQPRNGAGGETLDDDGKKKRTGTVWTASAHIITA
VIGSGVLSLAWSTAQLGWVVGPLTLMIFALITYYTSSSLLADCYRSGDQLTGKRNYTYM
DAVAAYLGRWQVLSCGVFQYVNLVGTAVGYTITASISAAAVHKANCFHNKGHAADCS
TYDTMYMVVFGIVQIFFSQLPNFSDLSWLSIVAAIMSFSYSSIAVGLSLARTISGRSGTT
TLTGTEIGVDVDSAQKVWLALQALGNIAFAYSYSMILIEIQDTVKSPPAENKTMKKATL
MGVTTTTAFYMLAGCLGYSAFGNAAPGNILTGFGFYEPYWLIDFANVCIVVHLVGAYQ
VFSQPIFAALETAAAKRWPNARFVTREHPLVAGRFHVNLLRLTWRTAFVVVSTVLAIVL

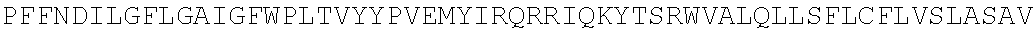

ASIEGVTESLKHYVPFKTKS
SEQ ID NO: 70: NP_001349744.1 uncharacterized protein
LOC100501686 [Zea mays](genomic)
ATGACGCAGCAGGACGTGGAGATGGCGGCGCGCCACGGGACCGGCGCCGACG
GAGCGGGATTCTACCCTCAGCCGCGGAACGGCGCCGGCGGCGAGACGCTCGAC
GACGACGGCAAGAAGAAGCGCACGGGAACGGTATGGACGGCAAGCGCGCACAT
CATCACAGCCGTCATCGGCTCCGGCGTGCTCTCCCTCGCCTGGTCGACTGCACA
GCTGGGCTGGGTCGTGGGGCCGCTCACCCTGATGATCTTTGCCTTGATCACGTA
CTACACCTCTAGCCTTCTTGCTGACTGCTACCGCAGCGGCGATCAGCTCACCGGC
AAGAGGAACTACACCTACATGGACGCTGTTGCCGCGTACCTGGGTCGATGGCAA
GTCCTGTCCTGTGGTGTTTTCCAGTATGTTAACTTGGTTGGAACTGCCGTTGGGTA
TACAATTACAGCGTCCATCAGTGCAGCGGCCGTGCACAAGGCAAACTGCTTCCAC
AACAAGGGCCACGCGGCCGACTGCAGCACCTACGACACCATGTACATGGTCGTA
TTTGGGATCGTTCAGATCTTCTTCTCTCAGCTCCCTAACTTCAGCGACCTTTCGTG
GCTGTCCATCGTCGCCGCCATCATGTCGTTCTCTTACTCCAGCATCGCCGTCGGC
CTCTCGTTGGCGCGGACCATTTCAGGCCGTAGTGGTACGACCACTCTGACCGGC
ACTGAGATCGGAGTCGACGTTGATTCAGCCCAGAAGGTCTGGCTCGCGCTTCAA
GCTCTTGGCAACATCGCGTTCGCTTACTCCTACTCCATGATTCTCATCGAAATCCA
AGACACGGTGAAGTCTCCTCCAGCCGAGAACAAGACGATGAAGAAGGCGACGCT
GATGGGCGTGACGACCACCACGGCGTTCTACATGCTTGCTGGCTGCCTCGGGTA
CTCGGCATTCGGGAACGCGGCGCCAGGGAACATCCTGACCGGGTTCGGCTTCTA
CGAGCCCTACTGGCTGATCGACTTCGCCAACGTCTGCATCGTGGTGCACCTGGT
GGGCGCGTACCAGGTCTTCTCCCAGCCCATCTTCGCGGCCTTGGAGACGGCGGC
CGCCAAGCGCTGGCCGAACGCCAGGTTCGTCACGCGCGAGCACCCCCTCGTGG
CCGGCAGGTTCCACGTCAACCTGCTCAGGCTGACGTGGAGGACGGCGTTCGT
GGTGGTGAGCACGGTGCTCGCCATCGTGTTGCCCTTCTTCAACGATATCCTGGGC

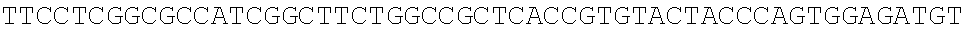

ACATCCGGCAGCGGCGTATACAGAAGTACACCAGCAGGTGGGTGGCGCTGCAGC
TGCTCAGCTTCCTGTGCTTCCTGGTCTCGCTCGCCTCGGCGGTCGCGTCCATCGA
GGGAGTCACCGAGTCGCTCAAACACTACGTTCCCTTTAAGACCAAGTCGTGA
SEQ ID NO: 71: PWZ08709 AAP3 [Zea mays] (protein)
MEVSSVEFGHAAAASKCFDDDGRLKRTGTMWTASAHIITAVIGSGVLSLAWAIAQLG
WVAGPTVMLLFSFVTYYTSALLADCYRSGDACTGKRNYTYMDAVNANLSGVKVWFC
GFLQYANIVGVAIGYTIAASISMLAIQRANCFHVEGHGDPCNISSTPYMIIFGVVQIFFSQ
IPDFDQISWLSILAAVMSFTYSTIGLGLGIAQVVSNKGVQGSLTGISVGAVTPVDKMWR
SLQAFGDIAFAYSYSLILIEIQDTIRAPPPSESKVMRRATVVSVAVTTFFYMLCGCMGYA
AFGDNAPGNLLTGFGFYEPFWLLDVANAAIAVHLVGAYQVYCQPLFAFVEKWARQR
WPKSRYITGEVDVPLPLGTAAGRCYKLSLFRLTWRTAFVVATTVVSMLLPFFNDVVGL

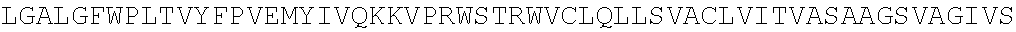

DLKVYKPFVTTS
SEQ ID NO: 72: PWZ08709.1 AAP3 [Zea mays] (protein)
ATGGAGGTGAGCTCCGTGGAGTTCGGTCACGCGGCGGCCGCCTCAAAGTGCTTT
GACGACGACGGTCGCCTCAAGCGCACAGGGACGATGTGGACGGCGAGCGCGCA
CATTATCACGGCCGTGATAGGGTCCGGGGTGCTGTCGCTCGCGTGGGCCATCGC
GCAGCTCGGCTGGGTGGCAGGCCCCACCGTCATGCTGCTCTTCTCCTTCGTCAC
CTACTACACGTCGGCCCTACTCGCCGACTGCTACCGCTCCGGCGACGCCTGCAC
CGGCAAGCGCAACTACACGTACATGGACGCGGTTAACGCCAATCTCAGTGGCGT
CAAGGTTTGGTTCTGCGGGTTCCTGCAGTACGCCAACATCGTCGGAGTCGCCATA
GGCTACACCATTGCCGCCTCTATTAGCATGCTGGCGATCCAGAGGGCGAACTGCT
TCCACGTGGAGGGGCACGGGGACCCCTGCAACATCTCCAGCACGCCCTACATGA
TCATCTTCGGCGTCGTGCAGATTTTCTTCTCGCAGATCCCGGACTTCGACCAGAT
ATCGTGGCTCTCCATCCTCGCCGCCGTCATGTCCTTCACCTACTCCACCATCGGC
CTGGGCCTGGGCATCGCGCAGGTGGTGTCCAACAAGGGCGTGCAGGGCAGCCT
GACGGGGATCAGCGTCGGCGCGGTCACCCCGGTCGACAAGATGTGGCGCAGCC
TGCAGGCGTTCGGCGACATCGCCTTCGCCTACTCCTACTCCCTCATCCTCATCGA
GATCCAGGACACCATCCGCGCGCCGCCGCCGTCCGAGTCCAAGGTCATGCGGC
GCGCCACCGTCGTCAGCGTGGCCGTCACCACGTTCTTCTACATGCTGTGCGGGT
GCATGGGGTACGCCGCGTTCGGGGACAACGCCCCCCGGGAACCTCCTCACGGGC
TTCGGCTTCTACGAGCCCTTCTGGCTCCTCGACGTCGCCAACGCCGCCATCGCC
GTGCACCTCGTCGGCGCCTACCAGGTCTACTGCCAGCCACTGTTCGCCTTCGTC
GAGAAGTGGGCGCGCCAGAGGTGGCCCAAGTCCCGCTACATCACGGGCGAGGT
CGACGTCCCGCTCCCGCTCGGGACCGCCGCCGGCCGGTGCTACAAGCTCAGCC
TGTTCCGGCTGACGTGGCGGACGGCGTTCGTGGTGGCCACGACGGTGGTGTCC

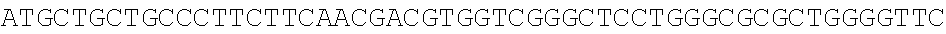

TGGCCGCTCACCGTCTACTTCCCCGTGGAGATGTACATCGTGCAGAAGAAGGTG
CCCAGGTGGAGCACGCGGTGGGTGTGCCTGCAGCTGCTCAGCGTCGCCTGCCT
CGTCATCACCGTCGCCTCCGCCGCAGGCTCCGTTGCCGGGATCGTCTCTGACCT
CAAAGTGTACAAACCGTTCGTCACCACCTCCTGA
SEQ ID NO: 73: NP_001149036 amino acid carrier [Zea mays] (protein)
MEVSSVEFGHHAAAASKCFDDDGRLKRTGTMWTASAHIITAVIGSGVLSLAWAIAQLG
WVAGPTVMLLFSFVTYYTSALLADCYRSGDACTGKRNYTYMDAVNANLSGVKVWFC
GFLQYANIVGVAIGYTIAASISMLAIQRANCFHVEGHGDPCNISSTPYMIIFGVVQIFFSQ
IPDFDQISWLSILAAVMSFTYSTIGLGLGIAQVVSNKGVQGSLTGISVGLVTPVDKMWR
SLQAFGDIAFAYSYSLILIEIQDTIRAPPPSESKVMRRATVVSVAVTTFFYMLCGCMGYA
AFGDNAPGNLLTGFGFYEPFWLLDVANAAIAVHLVGAYQVYCQPLFAFVEKWARQR
WPKSRYITGEVDVPLPLGTAGGRCYKLSLFRLTWRTAFVVATTVVSMLLPFFNDVVGL

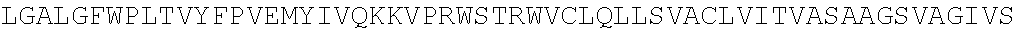

DLKVYKPFVTTS
SEQ ID NO: 74: NP_001149036 amino acid carrier [Zea mays] (genomic)
ATGGAGGTGAGCTCCGTGGAGTTCGGTCATCACGCGGCGGCCGCCTCAAAGTGC
TTTGACGACGACGGTCGCCTCAAGCGCACAGGGACGATGTGGACGGCGAGCGC
GCACATTATCACGGCCGTGATAGGGTCCGGGGTGCTGTCGCTCGCGTGGGCCAT
CGCGCAGCTCGGCTGGGTGGCAGGCCCCACCGTCATGCTGCTCTTCTCCTTCGT
CACCTACTACACATCGGCCCTACTCGCCGACTGCTACCGCTCCGGCGACGCCTG
CACCGGCAAGCGCAACTACACGTACATGGACGCGGTTAACGCCAATCTCAGTGG
CGTCAAGGTCTGGTTCTGCGGGTTCCTGCAGTACGCCAACATCGTCGGAGTCGC
CATAGGCTACACCATTGCCGCCTCTATTAGCATGCTGGCGATCCAGAGGGCGAAC
TGCTTCCACGTGGAGGGGCACGGGGACCCCTGCAACATCTCAGCACGCCCTACA
TGATCATCTTCGGCGTCGTGCAGATTTTCTTCTCGCAGATCCCGGACTTCGACCA
GATATCGTGGCTCTCCATCCTCGCCGCCGTCATGTCGTTCACCTACTCCACCATC
GGCCTGGGCCTGGGCATCGCGCAGGTGGTGTCCAACAAGGGCGTGCAGGGCAG
CCTGACGGGGATCAGCGTCGGCTTGGTCACCCCGGTCGACAAGATGTGGCGCAG
CCTGCAGGCGTTCGGCGACATCGCCTTCGCCTACTCCTACTCGCTCATCCTCATC
GAGATCCAGGACACCATCCGCGCGCCGCCGCCGTCCGAGTCCAAGGTCATGCG
GCGCGCCACCGTCGTCAGCGTGGCCGTCACCACGTTCTTCTACATGCTGTGCGG
GTGCATGGGGTACGCCGCGTTCGGGGACAACGCCCCCGGGAACCTCCTCACGG
GCTTCGGCTTCTACGAGCCCTTCTGGCTCCTCGACGTCGCCAACGCCGCCATCG
CCGTGCACCTCGTCGGCGCCTACCAGGTCTACTGCCAGCCCCTGTTCGCCTTCG
TCGAGAAGTGGGCGCGCCAGAGGTGGCCCAAGTCCCGCTACATCACGGGCGAG
GTCGACGTCCCGCTCCCGCTCGGGACCGCCGGCGGCCGGTGCTACAAGCTCAG
CCTGTTCCGGCTGACGTGGCGGACGGCGTTCGTGGTGGCCACGACGGTGGTGT

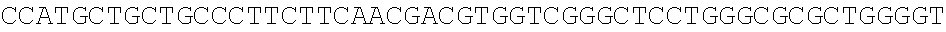

TCTGGCCGCTCACCGTCTACTTCCCCGTGGAGATGTACATCGTGCAGAAGAAGGT
GCCCAGGTGGAGCACGCGGTGGGTGTGCCTGCAGCTGCTCAGCGTCGCCTGCC
TCGTCATCACCGTCGCCTCCGCCGCAGGCTCCGTTGCCGGGATCGTCTCTGACC
TCAAAGTGTACAAACCGTTCGTCACCACCTCCTGA
SEQ ID NO: 75: ACG33909.1 amino acid carrier [Zea mays] (protein)
MEVSSVEFGHHAAAASKCFDDDGRLKRTGTMWTASAHIITAVIGSGVLSLAWAIAQLG
WVAGPTVMLLFSFVTYYTSALLADCYRSGDACTGKRNYTYMDAVNANLSGVKVWFC
GFLQYANIVGVAIGYTIAASISMLAIQRANCFHVEGHGDPCNISSTPYMIIFGVVQIFFSQ
IPDFDQISWLSILAAVMSFTYSTIGLGLGIAQVVSNKGVQGSLTGISVGAVTPVDKMWR
SLQAFGDIAFAYSYSLILIEIQDTIRAPPPSESKVMRRATVVSVAVTTFXYMLCGCMGY
AAFGDNAPGNLLTGFGFYEPFWLLDVANAAIAVHLVGAYQVYCQPLFAFVEKWARQR
WPKSRYITGEVDVPLPLGTAGGRCYKLSLFRLTWRTAFVVATTVVSMLLPFFNDVVGL

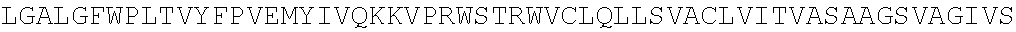

DLKVYKPFVTTS
SEQ ID NO: 76: ACG33909.1 amino acid carrier [Zea mays] (genomic)
ATGGAGGTGAGCTCCGTGGAGTTCGGTCATCACGCGGCGGCCGCCTCAAAGTGC
TTTGACGACGACGGTCGCCTCAAGCGCACAGGGACGATGTGGACGGCGAGCGC
GCACATTATCACGGCCGTGATAGGGTCCGGGGTGCTGTCGCTCGCGTGGGCCAT
CGCGCAGCTCGGCTGGGTGGCAGGCCCCACCGTCATGCTGCTCTTCTCCTTCGT
CACCTACTACACATCGGCCCTACTCGCCGACTGCTACCGCTCCGGCGACGCCTG
CACCGGCAAGCGCAACTACACGTACATGGACGCGGTTAACGCCAATCTCAGTGG
CGTCAAGGTCTGGTTCTGCGGCTTCCTGCAGTACGCCAACATCGTCGGAGTCGC
CATAGGCTACACCATTGCCGCCTCTATTAGCATGCTGGCGATCCAGAGGGCGAAC
TGCTTCCACGTGGAGGGGCACGGGGACCCCTGCAACATCTCCAGCACGCCCTAC
ATGATCATCTTCGGCGTCGTGCAGATTTTCTTCTCGCAGATCCCGGACTTCGACC
AGATATCGTGGCTCTCCATCCTCGCCGCCGTCATGTCCTTCACCTACTCCACCAT
CGGCCTGGGCCTGGGCATCGCGCAGGTGGTGTCCAACAAGGGCGTGCAGGGCA
GCCTGACGGGGATCAGCGTCGGCGCGGTCACCCCGGTCGACAAGATGTGGCGC
AGCCTGCAGGCGTTCGGCGACATCGCCTTCGCCTACTCCTACTCCCTCATCCTCA
TCGAGATCCAGGACACCATCCGCGCGCCGCCGCCGTCCGAGTCCAAGGTCATGC
GGCGCGCCACCGTCGTCAGCGTGGCCGTCACCACGTTCTTMTACATGCTGTGCG
GGTGCATGGGGTACGCCGCGTTCGGGGACAACGCCCCCGGGAACCTCCTCACG
GGCTTCGGCTTCTACGAGCCCTTCTGGCTCCTCGACGTCGCCAACGCCGCCATC
GCCGTGCACCTCGTCGGCGCCTACCAGGTCTACTGCCAGCCCCTGTTCGCCTC
GTCGAGAAGTGGGCGCGCCAGAGGTGGCCCAAGTCCCGCTACATCACGGGCGA
GGTCGACGTCCCGCTCCCGCTCGGGACCGCCGGCGGCCGGTGCTACAAGCTCA
GCCTGTTCCGGCTGACGTGGCGGACGGCGTTCGTGGTGGCCACGACGGTGGTG

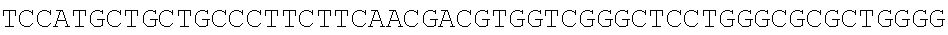

TTCTGGCCGCTCACCGTCTACTTCCCCGTGGAGATGTACATCGTGCAGAAGAAGG
TGCCCAGGTGGAGCACGCGGTGGGTGTGCCTGCAGCTGCTCAGCGTCGCCTGC
CTCGTCATCCCGTCGCCTCCGCCGCAGGCTCCGTTGCCGGGATCGTCTCTGA
CCTCAAAGTGTACAAACCGTTCGTCACCACCTCCTGA
SEQ ID NO: 77: NP_001142349 AAP2 [Zea mays] (protein)
MAENNVVATYYYPTAAPAAMEVCGAELGQGKPDKCFDDDGRPKRNGTMWTASAHII
TAVIGSGVLSLLGWAIAQLGWVAGPVVMLLFSLVTYYTSSLLADCYRSGDPSTGKRNYT
YMDAVNANLSGIKVQICGFLQYANIVGVAIGYTIAASISMLAIRRANCFHQKGHGNPCKI
SSTPYMIIFGVAEIFFSQIPDFDQISWWLSILAAVMSFTYSSIGLGLGVVQVIANRGVQGSL
TGITIGVVTPMDKVWRSLQAFGDVAFAYSYSLILIEIQDTIRAPPPSESTVMKRATVVSV
AVTTLFYMLCGCMGYAAFGDGAPGNLLTGFGFYEPFWLLDVANAAIVVHLVGAYQVY
CQPLFAFVEKWAAQRWPDSAYITGEVEVPLPLPASRRRCCKVNLFRATWRTAFVVAT

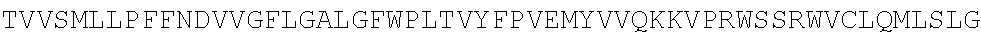

CLVISIAAAAGSIAGIASDLKVYRPFKSY
SEQ ID NO: 78: NP_001142349 AAP2 [Zea mays] (genomic)
ATGGCGGAGAACAACGTCGTGGCCACGTACTACTACCCGACGGCAGCGCCGGC
GGCCATGGAGGTCTGCGGCGCGGAGCTCGGCCAGGGCAAGCCCGACAAGTGCT
TCGACGACGATGGCCGCCCCAAGCGCAATGGGACGATGTGGACGGCGAGCGCG
CACATCATCACGGCGGTGATCGGCTCCGGGGTGCTCTCGCTGGGGTGGGCCATC
GCGCAGCTCGGCTGGGTGGCCGGACCCGTCGTCATGCTGCTCTTCTCGCTCGTC
ACCTACTACACCTCGTCGCTGCTCGCAGACTGCTACCGCTCCGGCGACCCCAGC
ACCGGCAAGCGGAACTACACCTACATGGACGCCGTCAACGCGAACCTCAGTGGC
ATCAAGGTCCAGATCTGCGGGTTCCTGCAGTACGCCAACATCGTGGGCGTGGCC
ATCGGCTACACCATCGCTGCCTCCATTAGCATGCTCGCGATCAGGAGGCCAACT
GCTTCCACCAGAAGGGACACGGCAACCCCTGCAAGATCTCCAGCACGCCCTACA
TGATCATCTTCGGCGTGGCGGAGATCTTCTTCTCGCAGATCCCGGACTTCGACCA
GATCTCCTGGCTCTCCATCCTCGCCGCCGTCATGTCCTTCACCTACTCCTCCATT
GGGCTCGGCCTGGGCGTCGTCCAAGTCATCGCGAACAGAGGCGTGCAGGGCAG
CCTGACCGGCATCACCATCGGCGTGGTGACCCCGATGGACAAGGTGTGGCGCAG
CCTCCAGGCGTTCGGCGACGTCGCCTTCGCCTACTCCTACTCCCTCATCCTGATC
GAGATCCAGGACACCATCCGGGCGCCGCCGCCGTCGGAGTCGACGGTGATGAA
GCGCGCCACGGTGGTGAGCGTGGCGGTCACCACGCTCTTCTACATGCTGTGCGG
CTGCATGGGGTACGCGGCGTTCGGCGACGGCGCGCCCGGGAACCTCCTCACGG
GCTTCGGCTTCTACGAGCCCTTCTGGCTCCTGGACGTGGCCAACGCCGCCATCG
TGGTCCACCTGGTCGGCGCCTACCAGGTCTACTGCCAGCCGCTGTTCGCCTTCG
TGGAGAAGTGGGCCGCGCAGCGGTGGCCGGACTCGGCGTACATCACCGGGGAG
GTCGAGGTCCCGCTCCCGCTCCCGGCGAGCCGGCGGCGGTGCTGCAAGGTGAA
CCTGTTCCGGGCGACGTGGCGGACGGCGTTCGTCGTGGCCACGACGGTCGTGT

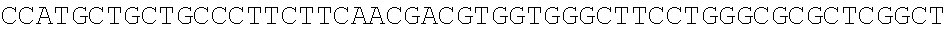

TCTGGCCGCTCACCGTCTACTTCCCCGTCGAGATGTACGTGGTGCAGAAGAAGGT
GCCGCGGTGGAGCTCCCGGTGGGTGTGCCTGCAGATGCTCAGCCTCGGCTGCC
TCGTCATCTCCATCGCCGCCGCAGCCGGGTCCATCGCCGGCATCGCGTCCGACC
TCAAAGTCTACCGCCCGTTCAAGTCCTACTGA
SEQ ID NO: 79: PWZO7549 AAP1 [Zea mays] (protein)
MTQQDVEMAARHGTGADGAGFYPQPRNGAGGETLDDDGKKKRTGVIATIGGVPSTG
ANVPPNVGVLDEPGTDAMPLMRPRTVWTASAHIITAVIGSGVLSLAWSTAQLGWVVG
PLTLMIFALITYYTSSLLADCYRSGDQLTGKRNYTYMDAVAAYLGRWQVLSCGVFQYV
NLVGTAVGYTITASISAAAVHKANCFHNKGHAADCSTYDTMYMVVFGIVQIFFSQLPNF
SDLSWLSIVAAIMSFSYSSIAVGLSLARTISGRSGTTTLTGTEIGVDVDSAQKVWLALQ
ALGNIAFAYSYSMILIEIQDTVKSPPAENKTMKKATLMGVTTTTAFYMLAGCLGYSAFG
NAAPGNILTGFGFYEPYWLIDFANVCIVVHLVGAYQVFSQPIFAALETAAAKRWPNARF

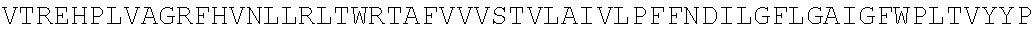

VEMYIRQRRIQKYTSRWVALQLLSFLCFLVSLASAVASIEGVTESLKHYVPFKTKS
SEQ ID NO: 80: PWZ07549 AAP1 [Zea mays] (genomic)
ATGACGCAGCAGGACGTGGAGATGGCGGCGCGCCACGGGACCGGCGCCGACG
GAGCGGGATTCTACCCTCAGCCGCGGAACGGCGCCGGCGGCGAGACGCTCGAC
GACGACGGCAAGAAGAAGCGCACGGGTGTAATAGCCACTATTGGAGGTGTACCA
AGCACTGGTGCAAATGTTCCGCCTAATGTTGGTGTCCTTGATGAGCCTGGCACTG
ATGCTATGCCACTCATGCGCCCTAGAACGGTATGGACGGCAAGCGCGCACATCAT
CACAGCCGTCATCGGCTCCGGCGTGCTCTCCCTCGCCTGGTCGACTGCACAGCT
GGGCTGGGTCGTGGGGCCGCTCACCCTGATGATCTTTGCCTTGATCACGTACTAC
ACCTCTAGCCTTCTTGCTGACTGCTCCGCAGCGGCGATCAGCTCACCGGCAAGA
GGAACTACACCTACATGGACGCTGTTGCCGCGTACCTGGGTCGATGGCAAGTCC
TGTCCTGTGGTGTTTTCCAGTATGTTAACTTGGTTGGAACTGCCGTTGGGTATACA
ATTACAGCGTCCATCAGTGCAGCGGCCGTGCACAAGGCAAACTGCTTCCACAACA
AGGGCCACGCGGCCGACTGCAGCACCTACGACACCATGTACATGGTCGTATTTG
GGATCGTTCAGATCTTCTTCTCTCAGCTCCCTAACTTCAGCGACCTTTCGTGGCTG
TCCATCGTCGCCGCCATCATGTCGTTCTCTTACTCCAGCATCGCCGTCGGCCTCT
CGTTGGCGCGGACCATTTCAGGCCGTAGTGGTACGACCACTCTGACCGGCACTG
AGATCGGAGTCGACGTTGATTCAGCCCAGAAGGTCTGGCTCGCGCTTCAAGCTCT
TGGCAACATCGCGTTCGCTTACTCCTACTCCATGATTCTCATCGAAATCCAAGACA
CGGTGAAGTCTCCTCCAGCCGAGAACAAGACGATGAAGAAGGCGACGCTGATGG
GCGTGACGACCACCACGGCGTTCTACATGCTTGCTGGCTGCCTCGGGTACTCGG
CATTCGGGAACGCGGCGCCAGGGAACATCCTGACCGGGTTCGGCTTCTACGAGC
CCTACTGGCTGATCGACTTCGCCAACGTCTGCATCGTGGTGCACCTGGTGGGCG
CGTACCAGGTCTTCTCCCAGCCCATCTTCGCGGCCTTGGAGACGGCGGCCGCCA
AGCGCTGGCCGAACGCCAGGTTCGTCACGCGCGAGCACCCCCTCGTGGCCGGC
AGGTTCCACGTCAACCTGCTCAGGCTGACGTGGAGGACGGCGTTCGTGGTGGTG
AGCACGGTGCTCGCCATCGTGTTGCCCTTCTTCAACGATATCCTGGGCTTCCTCG

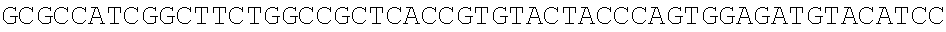

GGCAGCGGCGTATACAGAAGTACACCAGCAGGTGGGTGGCGCTGCAGCTGCTCA
GCTTCCTGTGCTTCCTGGTCTCGCTCGCCTCGGCGGTCGCGTCCATCGAGGGAG
TCACCGAGTCGCTCAAACACTACGTTCCCTTTAAGACCAAGTCGTGA
BRASSICA NAPUS
SEQ ID NO: 81: AKE34780 AAP8 [Brassica napus] (protein)
MKSLDTLHNPSAVESGNAAVKNVDDDGREKRTGTFLTASAHIITAVIGSGVLSLAWAL
AQLGWVAGTMILVIFAIITYYTSTLLADCYRAPDPITGTRNYTYMGVVRAYLGGKKVQL
CGLAQYGNLVGVSIGYTITASISLVAIGKANCFHGKGHGAKCTASNYPYMVAFGGLQIL
LSQIPNFHKLSFLSIIAAVMSFSYASIGIGLAIAKVASGKVGKTTLTGTVIGVDVSASDKV
WKAFQAVGDIAFSYAYTTILIEIQDTLRSSPPENKVMKKASLIGVSTTTVFYLLCGCIGY
AAFGNIAPGDFLTDFGFYEPFWLVIFANVCIAVHLVGAYQVYVQPFFQFVESKCNKKW

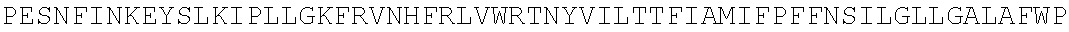

LTVYFPVAMHIAQTKVKKYSGRWLALNLLVLVCLIVSALAAVGSIVGLINNVKKYKPFES
ID
SEQ ID NO: 82: AKE34780 AAP8 [Brassica napus] (genomic):
Brara.F00660 | A06:3765107..3768058 forward

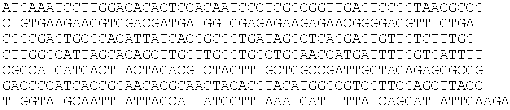

TTTTTTCAGTTTTATATAGAATTATGTTTCTGATCAACAATTTAGTCTACTGATATTA
ATAATTTTTGGTATTATATGGTAAAAATATTCTTGTGAAGATACATTTTTGATCTTTT
CCTATTTTTTCATAAGATGGTCCCAGGAACAAATTAAGATCAAAGTAATGTTTTCT
TGCAAGATCAAAGTAATCAACCATTTTTAGTGTATCCTATCTTTTGAGGAACATTAT
TTTGTGGTTCTAAATTTTTTTATTTTGAAAATTCTGCATGCTCTTCTTGGGAAGATAT
ATGAGTTAATTATCAAAATCTACAAAAAGATAAAATAATTATGAAATTTATCTTCTTT
CCAAAATTACTTAATGAATTGAATTGACTAGTGTAGGATTATCTCATTAAAGAATGC
TATCATTAAATTTTGATTGTTGGCCTCCCAAAAAAAAATTGAATTCAAATGAGAGAT
TGATCCAAACTTATCCACAAAAACAAAAGATTATCCGACTTTTTAACATCAAAGGAT
TAATGACAATAAACAATTTCGATGCTTAAGTCCTGCTTCGTGTAATCGCTGCTGTT
GATTGACAAAAACAAAGACTCCTATGTAATTTAGAAGAGTAACTAAGTTTTAGAATA

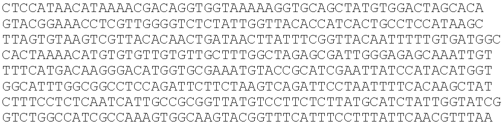

ATGTTTACATGAATCTTCAATGTTCGATCGAGCTAACGGTGGAAGTGGTATAATTA

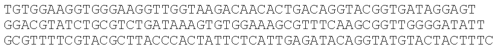

TTCTATCGTTCTTTGAATATTTGCATTTACATAGTAGTCATATATATGATGTATTAGT
TAGTGTATGGATCTATTAATGTTTTTTTTTTGCTCTTAGAAGTAATCTCTAACTACCG
ATTATGGATATATTAGTTAGAGAATATGGATCTACTACCACTTCTATTCTCTCTTTC
ACCAAAAAGGGATAAAGAAGAAGGTGGCATTTACCTTGAAGATAAGATGTTACTAT
CAACTAGAGTATTAGCCTAGTAGGCATGCATCTACAAAAAGGCTTGATGGATTTTT
TAATTATATATGTGAAGCTCTAAAGATACTGAAGCTCAAATATGTTTTTATTTTTTTT

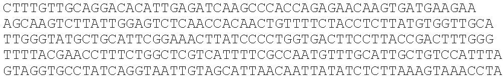

TCATTGACAAACAAACATGAGAACGTAGCACACATTTAAAAAGCAAAAACAGCTAA
TTATCACAACACACTTGTAATCTTCTTAAATATTCTTGTGTTATCCTCTGTTTTAGAA
ATTTAGATTAATAGTCGAAATTAGTAGAAATAGAGTTAGTTTGGTTTAAAATATAAT

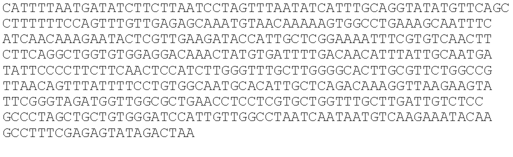

SEQ ID NO: 83: BnaA01g21750D [Brassica napus] (protein)
MKGFNTEQDHPAAESGNVYDVSDPTKNVDDDGREKRTGTWLTASAHIITAVIGSGVL
SLAWWAIAQLGWIAGTLILVIFSFITYFTSTMLADCYRAPDPVTGKRNYTYMDVVRSYLG
GRKVLCGVAQYGNLIGITVGYTITASISLVAVGKANCFHKKGHEADCTISNYPYMAVF
GIIQIILSIPNFHKLSFLSLMAAVMSFTYATIGIGLAIATVAGGKVGKTNMTGTVVGVDV
TAAQKIWRSFQAVGDIAFAYAYATVLIEIQASFHIKYLWNLVSFEYEPLDRIVDTLKSSP
AENKSMKRASLVGVSTTTFFYILCGCLGYAAFGNKAPGDFLTDFGFYEPFWLIDFANA
CIAFHLIGAYQVFAQPIFQFVEKRCNRNWPDNKFITSEYSVNVPFLGKFNISLFRLVWR

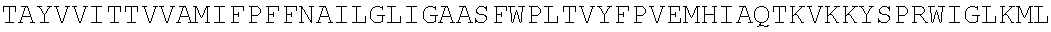

CWVCLIVSLLAAAGSIAGLISSVKTYKPFRTIHE
SEQ ID NO: 84: BnaA01g21750D [Brassica napus] (genomic):
>Brara.I01660 | A09:11198108..11202102 forward

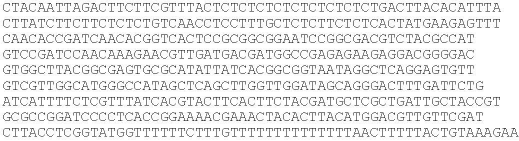

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

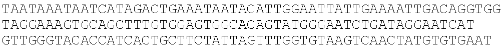

ATGTGATTTTACTTTATGACCGTTACCAAATGTACAGTTTAGTTATCTACCATATTG

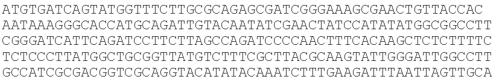

AAACAACAACTACTAACAATTATTGGTCATCAAGTTGTAGTTTTCAAAGTACCTATA
TCAATAATTGTAACAAGATAGATAATACAATAAAGTAACGGTTGATATGTTACGATA

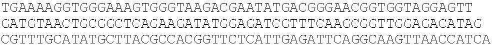

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

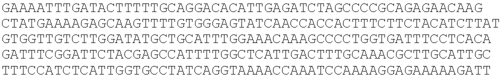

GTTTTCTTTTTGCACTTTCTCGCAGTTTAGCTAATGAAAAAGAAACATACTTCTTGC

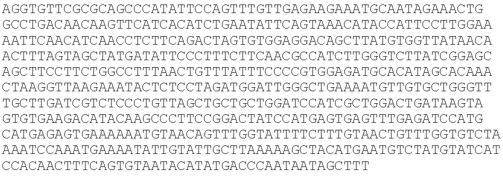

SEQ ID NO: 85: BnaA06g38000D [Brassica napus] (protein)
MKSFDAVHNPSAVESADANVDDDGREKRTGTLMTASAHIITAVIGSGVLSLAWAIAQL
GWVAGTLILVTFAVVNYYTSTMLADCYRSDAGARNYTYMDVVRSYLGGRKVQLCGLA
QYGCLVGVTIGYTITASISLVAIWKATCFHKKGHGAKCSIPNYPFMVAFGVVEIFLSQLP
NFHKLSFLSIIAAIMSFSYASIGIGLAISVVASGKVGKTSVTGTVVGVDVTASDKIWKAFQ
ATGDIAFSYSFSTILDTLRSNPPENKVMKKATLAGVSTTTVFYILCGCMGYAAFGNRAP
GDFLTDFGFYEPYWLINFANACIVLHLIAAYQVFAQPIFQLVENKCNKAWPENNFIHKE

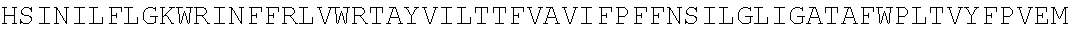

HISQRKVKKYSMKWNALKLLISVCLIVSLLAAIGSIVGLINSVKAYKPFHS
SEQ ID NO: 86: BnaA06g38000D [Brassica napus] (genomic)
Brara.F00658 A06:3747624..3750160 forward

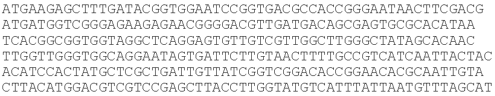

GTTTGAAAATATAAACAACATGGTATAGTTCATTGGCTTTGAAAGATTTACTTTATTT
TAGTTGTAAATAACTTAAGATTACTAAAATCGAATGAAGTTAGTTAGCATTAGTTTG
ATTTTGAAGATTTTTTTTTCGTCAAAAGTTGTAATCAATTTTTTTTATTTCTGTTTCG

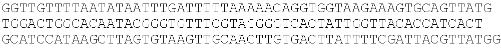

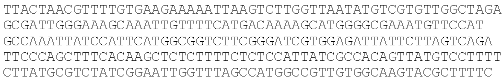

TCTCCCTTTTTTTTGTCAAAAAAATATACATTTGTAAAACATGCTCACGCAATTTCAA
AACCCTATGCAAATATCTTGACACATGCATTTAGTTGTTTAGTTTTCTGTTTTTACAT
AAAAATTTAGGAGCTCTGAGATACGTCTATACACATATTCAAAACTGATTATAAACT
GGGTTGACTTGTTCGATTGCATTAGTTGTCAATCTTTTTAAATGCCCTCGTACATAC
AAAAAGTTTGTTTAGGCAGAAAAGCATCTCTATCTAAGCCTCATATATTGTAGGATT
CTCACAGATTTGTAAGATGATATATGCATGTGATTTTTCTAACTAAAAAGGTGTGAT

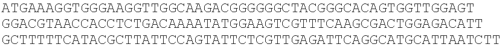

TTCTTTTATAGATGTTTTTGGTGGTCATCATCAAAATAGCTAGTGTGGCAATTTTTTT
ATATCATTATTGCTTTTTATTTGCATTTGAGTTTAAAAATCATATGATATATGTTGGT
TTGGTTAATAACTTTTGATGATGTTATTAATTATGATTCTTATCAAGAGAATATATAT
CTTGAATTTGACACAGATCACTTTAAATAATAATTAGCCTTCAACCGCGGTTCATAC

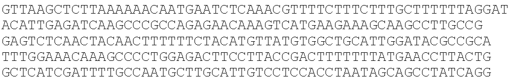

TAAACTCATAAGAATATAGCTTGAGTCTCAGATCAAGGATTGCTCGATTATAAAGA
AATAATTAGTACCTACTACTAACAACTTTGAAGTTACCGAAGTCTCAAGATGAAAAC

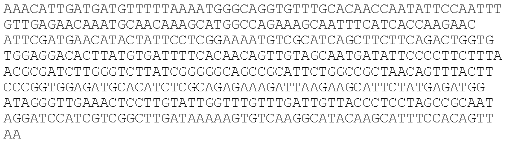

SEQ ID NO: 87: BnaA06g38010D [Brassica napus] (protein)
MKSFDTVESGDATGNNFDDDGREKRTGTLMTASAHIITAVVGSGVLSLAWAIAQLGW
VAGIVILVTFAVINYYTSTMLADCYRSDTGTRNCTYMDVVRAYLGGKKVQLCGLAQYG
CFVGVTIGYTITASISLVAIGKANCFHDKRHGAKCSMPNYPFMAVFGIVEIILSQIPSFHK
LSFLSIIATVMSFSYASIGIGLAMAVVASGKVGKTGATGTVVGVDVTTSDKIWKSFQAT
GDIAFSYAYSSILDTLRSSPPENKVMKKASLAGVSTTTFFYMLCGCIGYAAFGNKAPG
DFLTDFFYEPYWLIDFANACIVLHLIAAYQVFAQPIFQFVENKCNKAWPESNFITKEHS

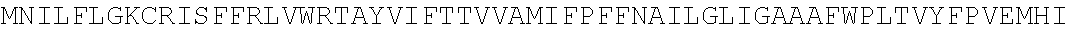

SQRKIKKHSMRWIGLKLLVLVCLIVTLLAAIGSIVGLIKSVKAYKHFHS
SEQ ID NO: 88: BnaA06g38010D [Brassica napus] (genomic)
>Brara.F00658 | A06:3747624..3750160 forward

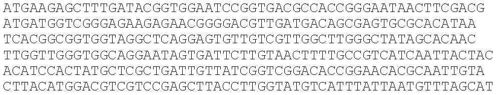

GTTTGAAAATATAAACAACATGGTATAGTTCATTGGCTTTGAAAGATTTACTTTATTT
TAGTTGTAAATAACTTAAGATTACTAAAATCGAATGAAGTTAGTTAGCATTAGTTTG
ATTTTGAAGATTTTTTTTTCGTCAAAAGTTGTAATCAATTTTTTTTATTTCTGTTTCG

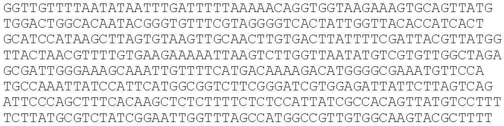

CTCTCCCTTTTTTTGTCAAAAAAATATACATTTGTAAAACATGCTCACGCAATTTCA
AAACCCTATGCAAATATCTTGACACATGCATTTAGTTGTTTAGTTTTCTGTTTTTACA
TAAAAATTTAGGAGCTCTGAGATACGTCTATACACATATTCAAAACTGATTATAAAC
TGGGTTGACTTGTTCGATTGCATTAGTTGTCAATCTTTTTAAATGCCCTCGTACATA
CAAAAAGTTTGTTTAGGCAGAAAAGCATCTCTATCTAAGCCTCATATATTGTAGGAT
TCTCACAGATTTGTAAGATGATATATGCATGTGATTTTTCTAACTAAAAAGGTGTGA

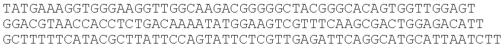

TTCTTTTATAGATGTTTTGGTGGTCATCATCAAAATAGCTAGTGTGGCAATTTTTTT
ATATCATTATTGCTTTTTATTTGCATTTGAGTTTAAAAATCATATGATATATGTTGGT
TTGGTTAATAACTTTTGATGATGTTATTAATTATGATTCTTATCAAGAGAATATATAT
CTTGAATTTGACACAGATCACTTTAAATAATAATTAGCCTTCAACCGCGGTTCATAC

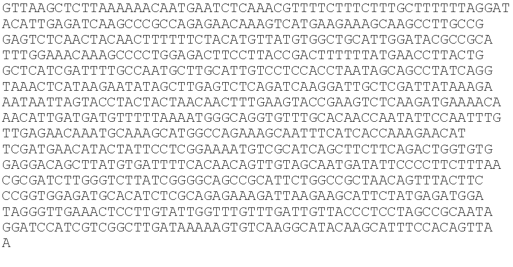

SEQ ID NO: 89: BnaA09g57230D [Brassica napus] (protein)
MKSYATEYNPSAVETAGNNFDDDGREKRTGTLMTATAHIITAVIGSGVLSLAWAIAQL
GWVAGTVILVTFAVINYFTSTMLADCYRSPDTGIRNYNYMDVVRAYLGGWWKVKLCGL
AQYGSLVGITIGYTITASISLVAIGKANCFHDKGHDAKCSVSNYPLMAAFGITQIVLSQIH
NFHKLSFLSIIATVMSFSYASIGIGLALAALASGKVGKTDLTGTVVGVDVTASDKIWRSF
QAAGDIAFSYAFSVVLVEIQACILSIRDDTLRSSPPENKVMKKASLAGVSTTTGFYILCG
CIGYAAFGNQAPGDFLTDFGFYEPYWLIDFANACIAVHLIAAYQVFAQPIFQFIEKKCNK

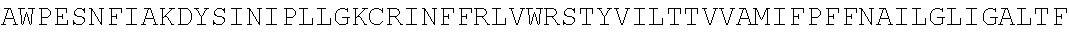

WPLTVYFPVEMHISQRKVKKYTMRWIGLKLLVLVCLVVSLLAAVGSIVGLISSVKAYKP
FHNLD
SEQ ID NO: 90: BnaA09g57230D [Brassica napus] (genomic)
ATGAAAAGCTACGCCACTGAGTATAATCCCTCGGCCGTGGAAACCGCCGGGAATA
ACTTCGACGATGATGGTCGGGAGAAGAGAACGGGGACGTTGATGACGGCGACCG
CGCACATAATCACGGCGGTGATAGGTTCTGGAGTCTTGTCGTTGGCTTGGGCTAT
AGCACAACTTGGTTGGGTGGCAGGAACGGTGATTTTGGTAACTTTTGCCGTTATA
AATTACTTCACATCTACAATGCTTGCGGACTGCTATCGATCTCCGGACACAGGAAT
ACGTAATTATAATTACATGGACGTTGTCAGAGCTTACCTTGGTGGTTGGAAAGTGA
AGCTGTGTGGACTGGCACAGTACGGGAGTCTAGTAGGGATCACTATTGGCTACAC
CATCACTGCCTCCATAAGCTTAGTAGCGATCGGGAAAGCAAATTGTTTTCATGACA
AGGGACATGATGCAAAATGTTCCGTATCAAATTATCCACTCATGGCGGCGTTTGGT
ATCACCCAGATTGTTCTTAGTCAGATTCATAATTTTCACAAGCTCTCTTTTCTCTCC
ATTATCGCTACCGTTATGTCCTTCTCTTATGCATCCATCGGAATTGGCTTAGCCTT
GGCTGCTCTGGCAAGTGGGAAGGTTGGTAAGACGGATCTGACGGGCACGGTGGT
TGGAGTAGACGTAACTGCGTCTGACAAAATATGGAGGTCGTTTCAAGCAGCTGGA
GACATTGCCTTTTCGTACGCATTTTCCGTTGTTCTCGTTGAGATTCAGGCATGCAT
TCTTTCAATTAGAGATGATACACTGAGATCAAGCCCACCAGAGAACAAAGTCATGA
AAAAAGCAAGCCTTGCTGGAGTTTCAACTACAACTGGTTTCTACATCTTGTGTGGC
TGGTTTTTATGAGCCTTACTGGCTCATTGATTTTGCTAATGCTTGCATTGCTGTCCA
CCTAATCGCAGCCTATCAGGTGTTTGCACAACCAATATTCCAGTTTATTGAGAAGA
AATGCAACAAAGCGTGGCCAGAAAGCAACTTCATCGCCAAAGATTATTCGATAAAC
ATACCATTGCTAGGGAAATGTCGCATCAACTTCTTCAGATTGGTCTGGAGGTCAAC
CTATGTGATTTTGACAACAGTTGTAGCGATGATATTCCCCTTCTTTAACGCGATCTT

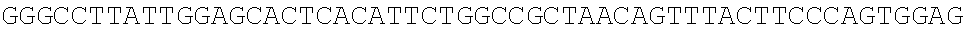

ATGCACATCTCGCAGAGAAAGGTTAAGAAGTATACTATGAGATGGATAGGGTTGA
AACTCCTTGTATTGGTTTGTTTGGTTGTTTCGCTCCTAGCTGCGTAGGATCCATT
GTCGGCTTGATAAGTAGTGTAAAGGCATACAAGCCTTTCCACAATTTAGATTAG
SEQ ID NO: 91: BnA09g57240D [Brassica napus] (protein)
MHRLYIDMSFTLHCLCFFSPLNMKTFDTSSAVESGTVAGNNVDDDGGEKRTGTLMTA
SAHIITAVIGSGVLSLAWAIAQLGWVAGTVLLVSFAVVVNYTSRMLADCYRSPDAGTR
NNTYMDVVRAYLGGRKVQLCGLAQYGSLVGMTIGYTITASISLVAIGKANCFHDKGHG
AKCLVSNYPAMAAFGIIQIVLSQIPNFHKLSFLSIIAAVMSFSYSSIGTGLALADLASGKV
GKTELTGTVVGVDVTASDKLWKSFQAAGNIAFSYAYSVVLVEIQACIFSTRNDTLSSSP
PENIVMKKASLVGVSTATAFYILCACMGYATFGSQAPGDLLTDFGFYEPYWLIDFANA
CIAVHLIGVYQQVIAQPIFQFVIKKCNKAWPESNFITLEHSMNIPLLGKCRINFFRLVWR

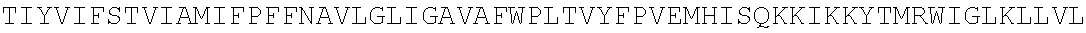

VCLIVSLLAAVGSIVGLISSLIRRKENMTLYISRLQFSHTHTHGPSTYPMINTNSYECLQ
NIISIDVCVHASSIYRYVIHSSSPMLLHISFLSSSVSPLKMKSFDTSSVVESGAGAGNNV
DDDCREKRTGTLITASAHIITTVIGSGVLSLAWAIAQLGWVVGTVILVAFAVIVNYTSRM
LADSYRSPEGTRNYTYMDVVRVYLGGRKVQLCGLAQFGSLVGVTIGYTITASISLVAIG
KANCFHDKGHGAKCSVSNYPLMAAFGIVQIFLSQIPNFHKLSFLSIIATVMSFSYASIGF
GLALAALASGKVGKTGLTGTVVGVDVTASDKLWKSFQAAGNIAFSYAYSVVLVEIQAC
IISINDDTLRSSPPENKVMKKASLAAVSTTTAFYILCGCIGYATFGNQAPGDFLTDFGFY
EPYWLIDFANACIAVHLIGAYQQVFAQPIFQFVEKKCNQAWPESNFITKEHSMNIPLLG
KCRINFFRLVWRTTYVIFSTVVAMIFPFFNAILGLIGAVAFWPLTVYFPVEMHISQKKVK
KYSVRWIVLKLLVLVCLIVSLLAAIGSIVSLISSVKAYKPFHNLD
SEQ ID NO: 92: BnaA09g57240D [Brassica napus] (genomic):
Brara.I05241 | A09:42950943..42954019 Brara.I05241 |
A09:42950943..42954019
reverse

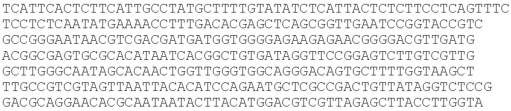

TGAACATATGTCGTTTTGTGACTCTATTACTTTGGTATTGTTTTAACCACAAAATAG
TATATTTCCAAAAAGAGCTATATTTCTTTTTTTTCTTAAAAAAAATCATATACTTTCAA
CCCTAAAAAGGAATCTGAAACAAGTACAAAGGCCGATTCACCAGGTGGCTCTAGG
TTACAAGGAGAGATTAACAAACAACAACAATGAACAGATTAACAAACAACAACAAT
GATATATATTTCCTTAGCTTAGTCTATTTTTTATTAAACAAAAACAAAAAACCATAAA

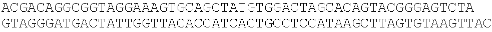

AACTTGTGATTTATTTTAATATATAACCTGTTCGTTTCACTTACGTGACCTATGACTA
AATGTCGTTGCTCGTGTGCATATGTCGCATGATCTTGTGACCAGTTGCATGTATTA
CAGCGACATGCAAACGGCCATAATGTCACATGGTTACCAACACGTTAAGAGAATA

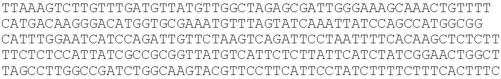

AAAAATATACGTGTAGAGCATTTTCAAGCTATTTCTAAACCCTATGCAAATATCTTG
ATACATACATCAATATTGTTTAATTTTCAGTAGAAATCATTCTCATTGATTTGTAAGA
TGATATATATTTATTACATATATGAATCTTCAAACTAATGACTAAAGCGGTATGATG

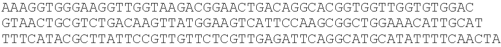

GAAATGTTGGTGGTCACACTCGGAATAGTGTAGCAATTTTTCCCTTTTGAAACACA
TTCTTTTATTTGCATTTATATTTTAATTACATGATATATATGTTGGTTGGAGTAATGA
CGGTTATTAGAGCACCATTAATCATAGTATTTTAGAAGGTTTATACTAATTAATTAA
AATAAAAAGGAATATTGAAAAAAGGAGAAGAACAACAAATAGCAAAGATACTTCAA
GAAAAAATTTGAGAAACTTTTCTATATGTGCAACTCATTTAGTAGTTGAGTTGTTTA
AAAGTAATTAAAGTATACTTAATAAAAGTAAATATTAATATTTTATTTTTGTTGAGAA
ACGCTTTTTCCTTGTTGATGATGGTCTATGTATGTGTAAAACAAAACGTTATTGGGA
TTCTTATCATTTTTTTTTGACACAGAACTATATTATCTTCACTTAATTAAATACGTCTT
CAACCGTCGTTTATGGTGGTTGTTTTCTGTTGCAATTCCCTTAAAGTATATATTGAT
GAAAGAGTTAATATGACGTATGCTAGCCCTTAATTAATTAATGACAGTATTGCTCAT

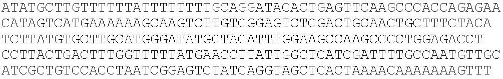

AAGATAGATAATTAGTATCTATCACTCACAATTTTTAAGTTTAAGTATAAGGCTCAA

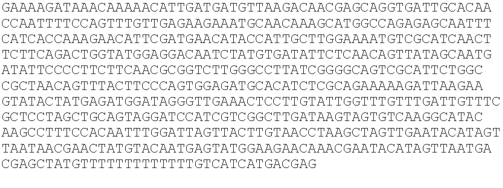

SEQ ID NO: 93: BnaC01g42990D [Brassica napus] (protein)
MKGFNTEQDHPAAESGNVYDVSDPTKNVDDDGREKRTGTWLTASAHIITAVIGSGVL
SLAWAIAQLGWIAGTLILVIFSFITYFTSTMLADCYRAPDPVTGKRNYTYMDVVRSYLG
GRKVQLCGVAQYGNLIGITVGYTITASISLVAVGKANCFHKKGHEADCTISNYPYMAVF
GIIQIILSQIPNFHKLSFLSLMAAVMSFTYATIGIGLAIATVAGGKVGKTNMTGTVVGVDV
TAAQKIWRSFQAVGDIAFAYAYATVLIEIQASFHIKYLWNLVSFEYEPLDRIVDTLKSSP
AENKSMKRASLVGVSTTTFFYILCGCLGYAAFGNKAPGDFLTDFGFYEPFWLIDFANA
CIAFHLIGAYQVFAQPIFQFVEKCRNRNWPDNKFITSEYSVNVLFLGKFNISLFRLVWR

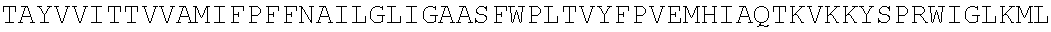

CWVCLIVSLLAAAGSIAGLISSVKTYKPFRTIHE
SEQ ID NO: 94: BnaC01g42990D [Brassica napus] (genomic) Brara.I01660 |
A09:11198108..11202102 forward

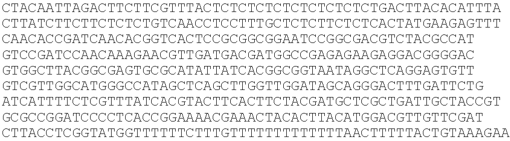

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGITT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

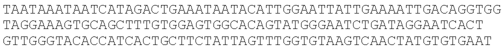

ATGTGATTTTACTTTATGACCGTTACCAAATGTACAGTTTAGTTATCTACCATATTG

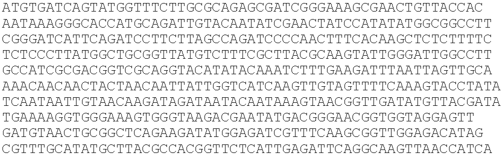

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

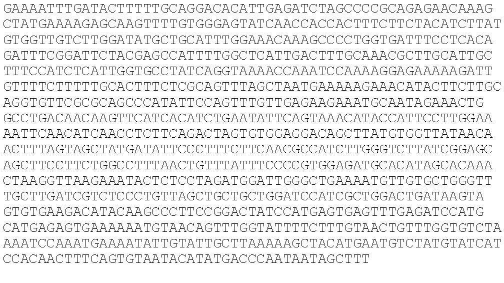

SEQ ID NO: 95: BnaC05g07760D [Brassica napus] (protein)
YGNLVGVSIGYTITASISLVAIGKANCFHGKGHGAKCTASNYPYMGAFGGLQILLSQIP
NFHKLSFLSIIAAVMSFSYASIGIGLAIAKVASGKVGKTTLTGTVIGVDVSASDKVWKAF
QAVGDIAFSYAYTTILIEIQDTLRSSPPENKVMKKASLIGVSTTTVFYLLCGCIGYAAFGN
IAPGDFLTDFGFYEPFWLVIFANVCIAVHLVGAYQVYVQPFFQFVESKCNKKWPESNFI

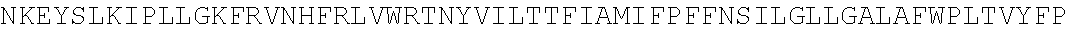

VAMHIAQTKVKKYSGRWLALHLLVLVCLIVSALAAVGSIVGLINNVKKYKPFESID
SEQ ID NO: 96: BnaC05g07760D [Brassica napus] (genomic)
TACGGCAACCTCGTTGGGGTCTCTATTGGTTACACCATCACTGCCTCCATAAGCTTA
GTAGCGATTGGGAAAGCAAATTGTTTTCATGGTAAGGGACATGGTGCGAAATGTAC
CGCATCGAATTATCCATACATGGGGGCATTTGGCGGCCTCCAGATTCTTCTAAGTCA
GATTCCTAATTTTCACAAGCTATCTTTCCTCTCAATCATTGCCGCGGTTATGTCCTTC
TCTTATGCATCTATTGGTATCGGTCTGGCCATCGCCAAAGTGGCAAGTGGGAAGGT
TGGTAAGACAACGCTGACAGGTACGGTGATAGGAGTGGACGTATCTGCGTCTGATA
AAGTATGGAAAGCGTTTCAAGCGGTTGGGGATATTGCGTTTTCGTACGCTTACACC
ACTATTCTCATTGAGATCCAGGACACATTGAGATCAAGCCCACCAGAGAACAAAGT
GATGAAGAAAGCAAGTCTTATTGGAGTCTCAACCACAACTGTTTTCTACCTCTTATG
TGGTTGCATTGGGTATGCTGCATTCGGAAACATAGCCCCTGGTGACTTCCTTACCG
ACTTTGGGTTTTACGAACCTTTCTGGCTCGTCATTTTCGCCAATGTTTGCATTGCTG
TCCATTTAGTAGGTGCCTATCAGGTATATGTTCAGCCCTTTTTCCAATTTGTTGAGAG
CAAATGCAACAAAAAGTGGCCTGAAAGCAATTTCATCAACAAAGAGTACTCGTTGA
AGATACCATTGCTCGGAAAATTTCGTGTCAACCACTTCAGGCTGGTGTGGAGGACA
AACTATGTGATTTTGACAACATTTATTGCAATGATATTCCCCTTCTTCAACTCCATCTT

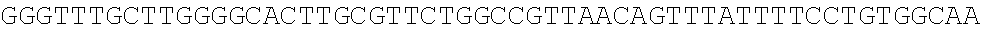

TGCACATTGCTCAGACAAAGGTTAAGAAGTATTCGGGTAGATGGTTGGCGCTGCAC
CTCCTCGTGTTGGTTTGCTTGATTGTCTCCGCCTTAGCTGCAGTGGGATCCATTGT
TGGCCTAATCAATAATGTCAAGAAATACAAGCCTTTCGAGAGTATAGACTAA
SEQ ID NO: 97: BnaC05g49200D [Brassica napus] (protein)
MKSFDAVHNPSAVESADANVDDDGREKRTGTLMTASAHIITAVIGSGVLSLAWAIAQL
GWVAGTLILVTFAIVNYYTSTMLADCYRSDAGARNYTYMDVVRSYLGGRKVQLCGLA
QYGCLVGITIGYTITASISLVAIWKATCFHKKGHGAKCSIPNYPFMAAFGVVEIFLSQLP
NFHKLSFLSIIAAVMSFSYASIGIGLAIAVVASGKVGKTGVTGTVVGVDVTASDKIWKAF
QATGDIAFSYSFSTILVEIQDTLRSSPPENKVMKKATLAGVSTTTVFYILCGCMGYAAF
GNRAPGDFLTDFGFYEPYWLINFANACIVLHLIAAYQVFAQPIFQLVENKCNKAWPEN

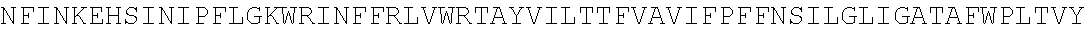

FPVEMHISQRKKEFMYGPNPNFKGSRTPTPSIQQRGDTGSGNSGAAVMITVLDQF
SEQ ID NO: 98: BnaC05g49200D [Brassica napus] (protein)
ATGAAAAGCTTTGACGCGGTGCATAATCCCTCTGCGGTGGAATCCGCTGACGCCA
ACGTCGACGATGATGGTCGGGAGAAGAGAACGGGGACGTTGATGACGGCGAGTG
CGCACATAATCACGGCGGTGATAGGTTCCGGAGTGTTGTCGTTGGCCTGGGCTAT
AGCACAGCTTGGTTGGGTGGCAGGAACACTGATTCTTGTAACTTTTGCCATCGTCA
ATTACTACACATCCACTATGCTCGCCGACTGTTATAGATCGGACGCAGGAGCTCGC
AACTATACGTACATGGACGTCGTCCGATCTTACCTTGGTGGTAGGAAAGTGCAGTT
ATGTGGACTGGCACAATACGGGTGTCTCGTAGGGATCACTATTGGTTACACCATCA
CTGCCTCTATAAGTTTAGTAGCGATTTGGAAAGCAACTTGTTTTCATAAAAAAGGAC
ATGGTGCGAAATGTTCCATCCCAAATTATCCATTCATGGCGGCCTTCGGGGTCGTG
GAGATTTTTCTTAGTCAGCTTCCTAATTTTCACAAGCTCTCTTTTCTCTCCATTATCG
CCGCCGTTATGTCATTCTCTTATGCGTCTATCGGAATTGGTTTAGCCATTGCCGTTG
TGGCAAGTGGAAAGGTTGGTAAGACGGGTGTGACGGGCACGGTGGTTGGAGTGG
ACGTGACCGCATCTGACAAAATATGGAAGGCGTTTCAAGCAACTGGAGACATTGCA
TTTTCATACTCTTTTTCCACTATTCTCGTTGAGATTCAGGATACATTGAGATCAAGCC
CACCAGAAAACAAAGTCATGAAAAAAGCAACACTCGCCGGAGTCTCAACGACAAC
TGTTTTCTACATCTTATGTGGCTGCATGGGATATGCTGCATTTGGAAACCGAGCCCC
CGGAGACTTCCTTACTGACTTTGGTTTTTATGAACCTTACTGGCTCATCAACTTTGC
CAATGCTTGCATCGTCCTCCACCTAATCGCAGCCTATCAGGTGTTTGCACAACCAA
TTTTCCAACTTGTTGAGAACAAATGCAACAAAGCATGGCCAGAAAACAATTTCATCA
ACAAAGAACATTCGATAAACATACCATTCCTCGGAAAATGGCGCATCAACTTCTTCA
GACTGGTGTGGAGGACAGCATATGTGATTTTGACAACATTTGTTGCAGTGATATTCC

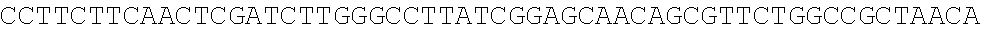

GTTTACTTCCCAGTGGAGATGCACATCTCGCAGAGAAAGAAGGAGTTCATGTATGG
TCCAAATCCTAACTTCAAAGGCTCTAGAACTCCAACACCGTCTATTCAACAACGAG
GAGACACTGGGAGTGGCAACTCCGGTGCTGCTGTGATGATCACGGTTCTAGA
TCAGTTTTGA
SEQ ID NO: 99: BnaC05g49210D [Brassica napus] (protein)
MKSFDTVESGDATGNNFDDDGREKRTGTLVTASAHIITAVVGSGVLSLAWAIAQLGW
VAGIVILVTFAVINYYTSTMLADCYRSDTGTRNCTYMDVVRAYLGGRKVQLCGLAQYG
CFVGVTIGYTITASISLVAIGKANCFHDKGHGAKCSMPNYPFMAAFGIVEIILSQIPSFHK
LSFLSIIATVMSFSYASIGIGLAMAVVASGKVGKTGVTGTVAGVDVTASDKIWKSFQAT
GDIAFSYAYSSILVEIQACILSSIDVLGVIIKIDTLRSSPPENKVMKKASLAGVSTTTFFYM
LCGCIGYAAFGNKAPGDFLTEFFYEPYWLIDYANACIVLHLIAAYQVFAQPIFQFVENK
CNKAWPESNFITIEHSMNIPFLGKCRVNFFRLVWRTAYVILTTVVAMIFPFFNSILGLIGA

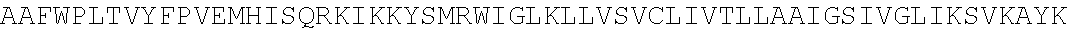

HFHS
SEQ ID NO: 100: BnaC05g4910D [Brassica napus] (genomic) Brara.F00658 |
A06:3747624..3750160 forward

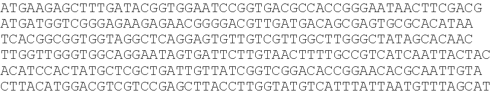

GTTTGAAAATATAAACAACATGGTATAGTTCATTGGCTTTGAAAGATTTACTTTATTT
TAGTTGTAAATAACTTAAGATTACTAAAATCGAATGAAGTTAGTTAGCATTAGTTTG
ATTTTGAAGATTTTTTTTTCGTCAAAAGTTGTAATCAATTTTTTTTATTTCTGTTTCG

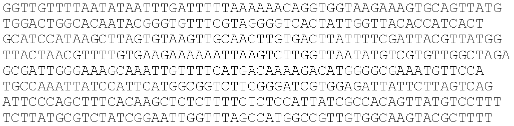

CTCTCCCTTTTTTTTGTCAAAAAAATATACATTTGTAAAACATGCTCACGCAATTTCA
AAACCCTATGCAAATATCTTGACACATGCATTTAGTTGTTTAGTTTTCTGTTTTTACA
TAAAAATTTAGGAGCTCTGAGATACGTCTATACACATATTCAAAACTGATTATAAAC
TGGGTTGACTTGTTCGATTGCATTAGTTGTCAATCTTTTTAAATGCCCTCGTACATA
CAAAAAGTTTGTTTAGGCAGAAAAGCATCTCTATCTAAGCCTCATATATTGTAGGAT
TCTCACAGATTTGTAAGATGATATATGCATGTGATTTTTCTAACTAAAAAGGTGTGA

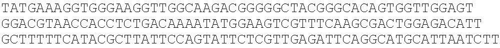

TTCTTTTATAGATGTTTTGGTGGTCATCATCAAAATAGCTAGTGTGGCAATTTTTTT
ATATCATTATTGCTTTTTATTTGCATTTGAGTTTAAAAATCATATGATATATGTTGGT
TTGGTTAATAACTTTTGATGATGTTATTAATTATGATTCTTATCAAGAGAATATATAT
CTTGAATTTGACACAGATCACTTTAAATAATAATTAGCCTTCAACCGCGGTTCATAC

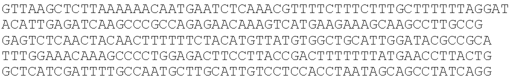

TAAACTCATAAGAATATAGCTTGAGTCTCAGATCAAGGATTGCTCGATTATAAAGA
AATAATTAGTACCTACTACTAACAACTTTGAAGTTACCGAAGTCTCAAGATGAAAAC

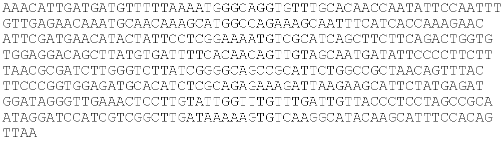

SEQ ID NO: 101: BnaC08g42410D [Brassica napus] (protein)
MKSFDTSSVVESGAGAGNNVDDDCREKRTGTLITASAHIITTVIGSGVLSLAWAIAQLG
WVVGTVILVAFAVIVNYTSRMLADSYRSPEGTRNYTYMDVVRVYLGGRKVQLCGLAQ
FGSLVGVTIGYTITASISLVAIGKANCFHDKGHGAKCSVSNYPLMAAFGIVQIFLSQIPN
FHKLSFLSIIATVMSFSYASIGFGLALAALASGKVGKTGLTGTVVGVDVTASDKLWKSF
QAAGNIAFSYAYSVVLVEIQACILSINDDTLRSSPPENKVMKKASLAAVSTTTAFYILCG
CIGYATFGNQAPGDFLTDFGFYEPYWLIDFANACIAVHLIGAYQQVFAQPIFQFVEKKC
NQAWPESNFITKEHSMNVPLLGKCRINFFRLVWRTTYVIFSTVVAMFPFFNAILGLIGA

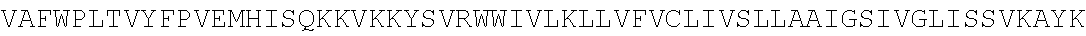

PFHNLD
SEQ ID NO: 102: BnaC08g42410D [Brassica napus] (genomic): Brara.I05240 |
A09.42945936..42949113 reverse

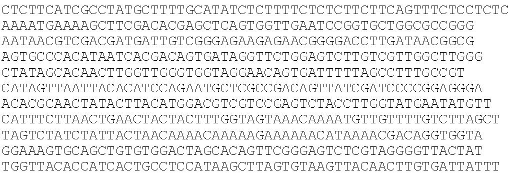

TGATATATAAAACATGTTTGTTTCAATTTCTAACGTGACCTACGACTAAGTATTGCT
CACATGGCCATAATGTCATATGGTTACCAATATGTTAAGAAAATATTTAAGTCTGGT

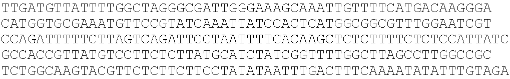

ACATTCTCAAGCTATTTCTAAACCCTATGCAAATATCTTGATACATACTTAATACATT
TATATTTTAGTTTTCAGTACAAATCTTTCTTTTTCAGTAGAAATCATCCTCATTGATT
TGTAAGATGATATATATTAACTATTTACACATCTATTGTTTAAAAAAAAAAAATTTAT

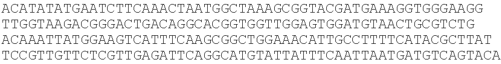

CTCAGAAATCAGAATAGTGTAGCATTTTCTAATATTACAGTGAAACTTCTATAAATT
AATAATGTTGGGACTACATCAAAACTATAATTTTTTTATTAATTTATAGAGATACTAA
TTTATCGATATACTAATAGAACCAAAAACTCAATTTGAAACTATAAAATTATATTATT
TTATAGATTTTTAGTATATATTAATTTATAGATTATTAATTTAAAGAGGTTATACTGTA
GTTTTTTTATTCTTTTATTTACATGATATATATTTTGGTTGGAATAATGACTGTTATTA
GAGCACCATTAGTCATAGTATTAGTATCGTAGGGGGTGTCTAATAATTAAAATAAA
AAGGAATATTGAAAAAGAGAAGAACAGAAAATAGCAAAAACGATTCTTGTTGACA
TACTTCAAGAAAAAAAAGTCCGATTTTTTTACAAGTGTAACTCATTTATTAGTTGGG
TTGTTTAAAAGTAATTAAAGTATACTTGTAAAACTAAATATTTTTGGCACCGAACTAT
ACTATTATTTTCACTACAATACGTCTTCAACCGTCGTTTATGGTGGTTGTTTTCTTG
TTGTAATTCCCTTAAAGTATATATTGATGGATGATTAGAGTTAATATGATGTATGTT

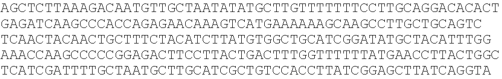

ACTCATAAAAAAGGAGAAATACTTTAGAATATTACTAAAAACAGCTTACTATTCTAAA
TTAACACACGCAAAATGATCAAAATAACATTAACTAAAATTTAAAAATATACTTTTAT
TTTATAGTTGGGTTTAGGTTTAGTGAATAGAGTTTAGGGGTTAGTATTTAAAAAGTG
GAAGTGCAGAGTTTGAAATGTTTTTTGTCATTTTCTCCTTATGTGATAATTTTGTCA
TAATATTTTTTTGTGGTATCTAAGTCATTTGTCCTAAAAAAGTCAAGAGTTTAATAT

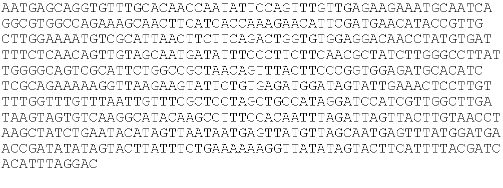

SEQ ID NO: 103: BnaC08g42420D [Brassica napus] (protein)
MHRLYIDMSFTLHCLCFFSPLNMKSFDTSSAVESGTVAGNNVDDDGGEKRTGTLMTA
SAHIITAVIGSGVLSLAWAIAQLGWVAGTVLLVSFAVVVNYTSRMLADCYRSPDAGTR
NNTYMDVVRAYLGGRKVQLCGLQAYGSLVGMTIGYTITASISFVAIGKANCFHDKGHG
AKFSVSNYPAMAAGFIIQIVLSQIPNFHKLSFLSIIAAVMSFSYSSIGTGLALADLASGKV
GKTELTGTVVGVDVTASDKLWKSFQAAGNIAFSYAYSVVLVEIQACIFSTRNDTLSSSP
PENIVMKKASIVGVSTATAFYILCACMGYATFGSQAPGDLLTDFGFYEPYWLIDFANAC
IAVHLIGAYQQVIAQPIFQFVEKKCNKAWPESNFITKEHSMNIPLLGKCRINFFRLVWRT

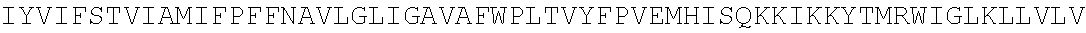

CLIVSLLAAVGSIVGLISSVKAYKPFHNLD
SEQ ID NO: 104: BnaC08g4240D [Brassica napus] (genomic): Brara.I05241 |
A09:42940943..42954019 reverse

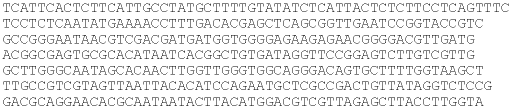

TGAACATATGTCGTTTTGTGACTCTATTACTTTGGTATTGTTTTAACCACAAAATAG
TATATTTCCAAAAAGAGCTATATTTCTTTTTTTTCTTAAAAAAAATCATATACTTTCAA
CCCTAAAAAGGAATCTGAAACAAGTACAAAGGCCGATTCACCAGGTGGCTCTAGG
TTACAAGGAGAGATTAACAAACAACAACAATGAACAGATTAACAAACAACAACAAT
GATATATATTTCCTTAGCTTAGTCTATTTTTTATTAAACAAAAACAAAAAACCATAAA

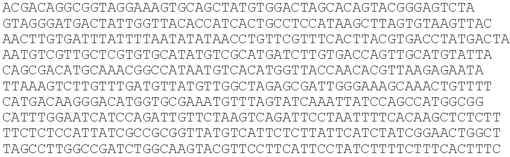

AAAAATATACGTGTAGAGCATTTTCAAGCTATTTCTAAACCCTATGCAAATATCTTG
ATACATACATCAATATTGTTTAATTTTCAGTAGAAATCATTCTCATTGATTTGTAAGA
TGATATATATTTATTACATATATGAATCTTCAAACTAATGACTAAAGCGGTATGATG

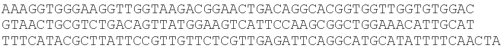

GAAATGTTGGTGGTCACACTCGGAATAGTGTAGCAATTTTTCCCTTTTGAAACACA
TTCTTTTATTTGCATTTATATTTTAATTACATGATATATATGTTGGTTGGAGTAATGA
CGGTTATTAGAGCACCATTAATCATAGTATTTTAGAAGGTTTATACTAATTAATTAA
AATAAAAAGGAATATTGAAAAAAGGAGAAGAACAACAAATAGCAAAGATACTTCAA
GAAAAAATTTGAGAAACTTTTCTATATGTGCAACTCATTTAGTAGTTGAGTTGTTTA
AAAGTAATTAAAGTATACTTAATAAAAGTAAATATTAATATTTTATTTTTGTTGAGAA
ACGCTTTTTCCTTGTTGATGATGGTCTATGTATGGTGTAAAACAAAACGTTATTGGGA
TTCTTATCATTTTTTTTTGACACAGAACTATATTATCTTCACTTAATTAAATACGTCTT
CAACCGTCGTTTATGGTGGTTGTTTTCTGTTGCAATTCCCTTAAAGTATATATTGAT
GAAAGAGTTAATATGACGTATGCTAGCCCTTAATTAATTAATGACAGTATTGCTCAT

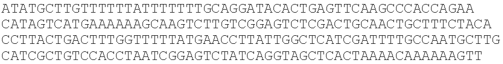

TAAGATAGATAATTAGTATCTATCACTCACAATTTTTAAGTTTAAGTATAAGGCTCA

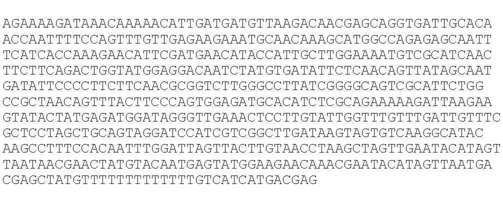

SEQ ID NO: 105: BnaC08g42430D [Brassica napus] (protein)
MKSFHTEYNPSAVEAAGNNFDDDGREKRTGTVMTASAHIITAVIGSGVLSLAWAIAQL
GWVAGTVILVTFAVINYFTSTMLADCYRSPDTGIRNYNYMDVVRAYLGGWKVKLCGL
AQYGSLVGITIGYTITASISLVAIGKANCFHEKGHGAKCSVSNYPLMAAFGIIQIVLSQIH
NFHKLSFLSIIATVMSFSYASIGIGLALAALASGKVGKTDLTGTVVGVDVTASDKIWRSF
QAAGDIAFSYAFSVVLVEIQACILSIRDDTLRSSPPENKVMKKASLAGVSTTTGFYILCG
CIGYAAFGNQAPGDFLTDFGFYEPYWLIDFANACIAVHLIAAYQVFAQPIFQFIEKKCNK

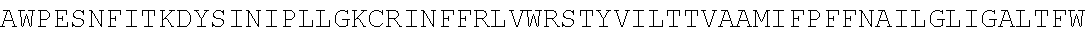

PLTVFPVEMHISQKKVKKYTMRWIGLKLLVLVCLVVSLLAAVGSIVGLISSVKAYKPFH
NLD
SEQ ID NO: 106: BnaC08g42430D [Brassica napus] (genomic)
ATGAAAAGCTTCCACACTGAGTATAATCCCTCGGCCGTGGAAGCCGCCGGGAATA
ACTTCGACGACGATGGTCGGGAGAAGAGAACGGGGACGGTGATGACGGCAAGT
GCTCACATTATCACTGCTGTGATAGGTTCCGGAGTCTTGTCCTTGGCTTGGGCTAT
AGCACAACTTGGTTGGGTGGCAGGAACAGTGATTTTGGTAACTTTTGCCGTTATAA
ATTACTTCACATCTACAATGCTTGCCGACTGTTATCGATCTCCGGACACAGGAATA
CGTAATTATAATTACATGGACGTTGTCAGAGCTTACCTTGGTGGTTGGAAAGTGAA
GCTATGTGGTCTGGCACAGTACGGGAGTCTAGTAGGGATCACTATTGGTTACACC
ATCACTGCCTCCATAAGCTTAGTAGCGATAGGGAAAGCAAATTGTTTTCATGAAAA
GGGACATGGTGCAAAATGTTCCGTATCGAATTATCCACTCATGGCGGCGTTTGGT
ATCATCCAGATTGTTCTTAGTCAGATTCATAATTTTCACAAGCTCTCTTTTCTCTCC
ATTATCGCCACCGTTATGTCCTTCTCTTATGCATCCATCGGAATTGGCTTGGCCTT
GGCCGCTCTGGCAAGTGGGAAGGTTGGTAAGACGGATCTGACGGGCACGGTGG
TTGGAGTAGACGTAACTGCGTCTGACAAAATATGGAGGTCGTTTCAAGCAGCTGG
AGACATTGCCTTTTCGTACGCATTTTCCGTTGTTCTCGTTGAGATTCAGGCATGCA
TTCTTTCAATTAGAGATGATACACTGAGATCAAGCCCACCAGAGAACAAAGTCATG
AAAAAAGCAAGCCTTGCTGGAGTTTCAACTACAACTGGTTTCTACATCTTATGTGG
CTGCATCGGATATGCTGCTTTTGGAAACCAAGCCCCTGGAGACTTCCTAACTGAC
TTTGGTTTTTATGAGCCTTACTGGCTCATTGATTTTGCTAATGCTTGCATTGCTGTC
CACCTAATCGCAGCCTATCAGGTGTTTGCACAACCAATATTCCAGTTTATTGAGAA
GAAATGCAACAAAGCGTGGCCAGAAAGCAACTTTATCACCAAAGATTATTCGATAA
ACATACCATTGCTAGGGAAATGTCGCATCAACTTCTTCAGATTGGTCTGGAGGTCA
ACCTATGTGATTTTGACAACAGTTGCAGCAATGATATTCCCCTTCTTCAACGCGAT

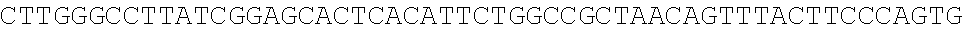

GAGATGCACATCTCGCAGAAAAAGGTTAAGAAGTATACTATGAGATGGATAGGGT
TGAAACTCCTTGTATTGGTTTGTTTGGTTGTTTCGCTCCTAGCTGCAGTAGGATCC
ATTGTCGGCCTCATAAGTAGTGTAAAGGCATACAAGCCTTTCCACAATTTAGATTA
G
SEQ ID NO: 107: BnaCnng14480D [Brassica napus] (protein)
MEKKSMFIEQSFTDHKSGDMNKNFDDDGRQKRTGTWMTGSAHIITAVIGSGVLSLAW
AIAQLGWVAGPAVLMAFSFITYFTSTMLADCYRSPDPVTGKRNYTYMEVVRSYLGGR
KVMLCGLAQYGNLIGITIGYTITASISMVAVKRSNCFHKNGHNVKCSTSNTPFMIIFACI
QIVLSQIPNFHNLSWLSILAAVMSFSYASIGVGLSIAKVAGGGVHARTALTGVTVGVDV
TGSDKVWRTFQAVGDIAFAYAYSTDTLKASPPSENKAMKRASLVGVSTTTFFYMLCG
CVGYAAFGNNAPGNFLTGFGFYEPFWLIDFANVCIAVHLVGAYQVFCQPIFQFVSQS
AKRWPDNKFITGEYKMNVPCGGDFGISLFRLVWRTSYVVVTAVVAMIFPFFNDFLGLI

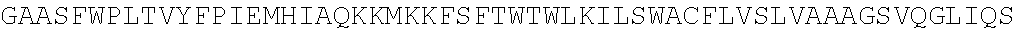

LLNADLFTKSVAPES
SEQ ID NO: 108: BnaCnng14480D [Brassica napus] (genomic): Brara.B01675 |
A02:9628389.9631130 reverse

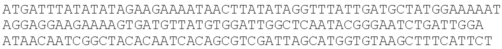

TTCTTTCAGAATGATAGTCAATAATAGCAGGCTCTCCTTTTTCACCTATTTTCACCC
ACGTTTATTATGTTAGGACAGGTGACTAACTCTTTTTTATAATTATTAATTTTACCTT
TTAAAGAACAGATGCTATGATAGGTAAGAGATATGATATATAATATCTACAAAAGTT
TTTCTTTGTCACAAGTTATTTGATATGTACAGAGTAATATAAATTTAAATTCTATTGA
GTGTGGGAGTCGAAAGGAGCTCAAATTTTCAAAGTGAAAAGTTAGATCTAGTAGG
ATCGTTGAGATTTTGTATTCTAAATTTATCAAATTTTTTTTGTCTGGAACTTTATATA
TTTATAATTATTAAGGAATGGGTTTTAAAGTACGAAAGAAAGAAAAAATTAAAATG

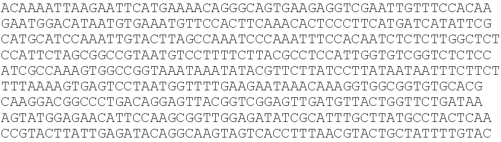

TCACCAGTTTAATTTAAAATGTTTTTTAAACATCTCGCACACCTGTTAAGAAAGGAG
TATTAGTTTTTCACTATAACCCTTATTAAATGTTTCAGCTAATACAAATGGTATCTTT
GGAAAAAATAATAATATACTCAGACCTGAATATACTACATATTTTTATAATTTAATAT
AACGGAAAATATGGTTATAATTGAAAGTTGAAACTTATGTTAAAACTTTGTATTGGC
AACTCTAAAACTAAACTCAGTTTTAAAAAATTAGCCATAAACTGACACTTCTGAAA
TGGAAGGATTATGTTTAGAGCTGAGTTTTAGAGACCGCGATAAGAGAGAGCCAAA
AAAAAATTAAACGTGTGTCGGTTTGTGACAAAGTAAAGGCCACTTCAGATGAATTA
TTATTTGTAGTAAAACATGAGAGGAAACCAGAGTCTAACTAGTAGGCTTTTATTCAA
TAAATAAATACTTATAAAATGATTTAACTTTGAGTACGGTTTACAACTGATGTTTTG
GCTTCTTTTGTACACAAAAGATTAATATTCTAACTTTAATTAATGTATTTCATTTTAA
AAGTAAAAAAAAATCTAAAAATATGGATTGTAGAATTTTATTGGAAAAAAACAAAAC
TAACAAAAACTAATTAATCAAAAATGACTCTTGACTTAGTTAATTTTTATACTTTTATA
TTTATCACTAAATAACATTAAAGTCACCAATTACGTATTGTCATTTCAGATAATTGTA
AACGATTTAGTGAACTACATTTTGTGTGTGTTTTGATCTACCACTACTAAAGTATGT

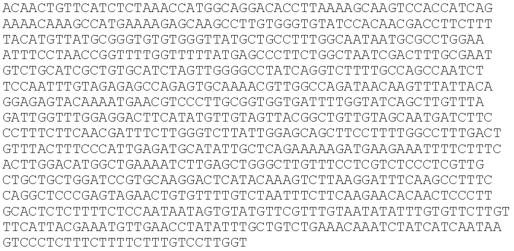

SEQ ID NO: 109: BnaCnng25620D [Brassica napus] (protein)
MKSFNTDQHGHSAAESADVYAMSDPTKNVDDDGREKRTGTWLTASAHIITAVIGSGV
LSLAWAIAQLGWIAGTLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
VTAAQKIWRSFQAVGDIAFAYAYATDTLRSSPAENKAMKRASFVGVSTTTFFYILCGC
LGYAAFGNKAPGDFLTDFGFYEPFWLIDFANACIAFHLIGAYQVFAQPIFQFVEKKCNR

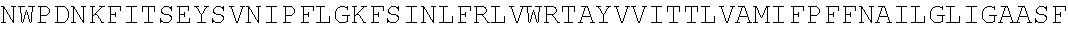

WPLTVYFPVEMHIAQTKVKKYSSRWIGLKMLCWVCLIVSLLAAAGSIAGLISSVKTYKP
FRTIHE
SEQ ID NO: 110: BnaCnng25620D [Brassica napus] (genomic): ara.I01660 |
A09:11198108..11202102 forward

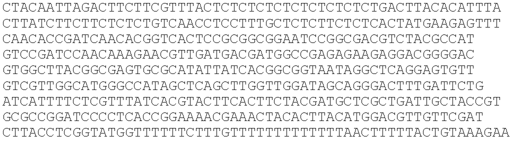

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

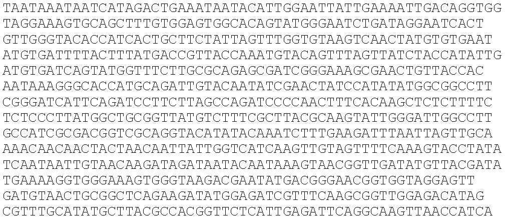

ATCAATTTTACTATTGAGTCTTGTTTGCTTTGAATATGAACCTCTTAATAGAATTGT
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

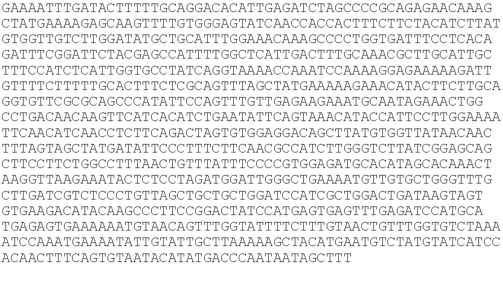

SEQ ID NO: 111: CAD92449 AAP1 [Brassica napus] (protein)
MKSFNTDQHGHSAAESGDVYAMSDPTKNVDDDGREKRTGTWLTASAHIITAVIGSGV
LSLLAWAIAQLGWIAGTLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
GIIQILLSQIPNFHKLSFLSLMAAVMSFAYASIGIGLLAIATVAGGKVGKTNMTGTVVGVD
VTAAQKIWRSFQAVGDIAFAYAYATVLIEIQDTLRSSPAENKAMKRADFVGVSTTTFFYI
LCGCLYGYAAFGNKAPGDFLTNFGFYEPFWLIDFANACIAFHLIGAYQVFAQPIFQFVEK
KCNRNWPDNKFITSEYSVNIPFLGKFSINLFRLVWRTAYVVITTLVAMIFPFFNAILGLIG

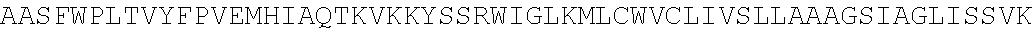

TYKPFRTIHE
SEQ ID NO: 112: CAD92449 AAP1 [Brassica napus] (genomic): Brara.I01660 |
A09:11198108..11202102 forward

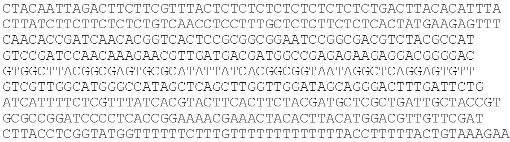

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAATGACAAAATATATATTAGTAA
TAAAAACATATAATATAATTTATAAGCGACAGTAACTTTTAGGTCAATTTGAAATAAA
ACTGGTTATGTAATGAGTTTATATAGAACAATGATGGTGGTTTATAAAATAGTTGAT
GTTACGAACTATAAGATCAATCATAAGAAATCATCATTGATATCTTTTGAAACAATC
GAAAAGCTTATGCATCCGATGAGTTGTGGTTAGGAATGTAGATAAAGTAATGGATT
TATAGATTTTCTAACATTTCCTGCCAATTTGGTTTTACAGAAGAAAAAAAATCTATC
GGGCATATAAATTACTGTTGCGTATAAATTGATTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

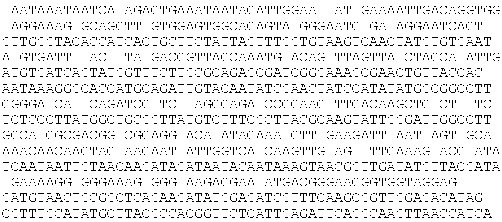

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

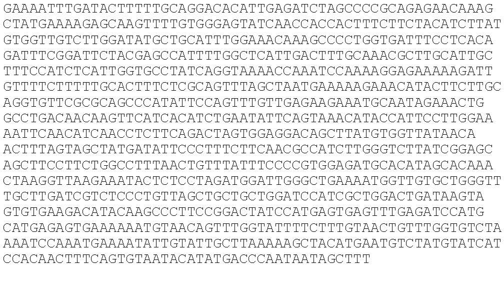

SEQ ID NO: 113: XP_013645981 AAP8-like [Brassica napus] (protein)
MKSLDTLHNPSAVEGNAAVKNDDDGREKRTGTFLTASAHIITAVIGSGVLSLAWAL
AQLGQVAGTMILVIFAIITYYTSTLLADCYRAPDPITRTRNYTYMGVVRAYLGGKKQLL
CGLAQYGNVGVSIGYTITASISLVAIGKANCFHGKGHGAKCTASNYPYMGAFGGLQIL
LSQIPNFHKLSFLSIIAAVMSFSYASISISLLGLAIAKVASGKVGKTTLTGTVIGVDVSASDKV
WKAFQAVGDIAFSYAYTTILLIEIQDTLRSSPPENKVMKKASLIGVSTTTVFYLLCGCIGY
AAFGNLSPGDFLTDFGFYEPFWLVIFANVCIAVHLVGAYQVYVQPFFQFVESKCNKK

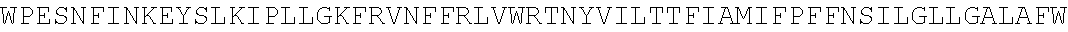

PLTVFPVAMHIAQTKVKKYSGRWLALNLLVLVCLIVSALAAVGSIVGLINNVKKYKPFE
SID
SEQ ID NO: 114: XP_013645981 AAP8-like [Brassica napus] (genomic)
ATGAAATCCTTGGACACACTCCACAATCCCTCGGCGGTTGAGTCCGGTAACGCCG
CTGTGAAGAACGTCGACGATGATGGTCGAGAGAAGAGAACGGGGACGTTTCTGA
CGGCGAGTGCGCACATTATCACGGCGGTGATAGGCTCAGGAGTGTTGTCTTTGG
CTTGGGCATTAGCACAGCTTGGTTGGGTGGCTGGAACCATGATTTTGGTGATTTT
CGCCATCATCACTTACTACACCTCTACTTTGCTCGCCGATTGCTACAGAGCGCCG
GACCCCATCACCAGAACACGCAACTACACGTACATGGGCGTCGTTCGAGCTTACC
TTGGTGGTAAAAAGGTGCAGCTATGTGGACTAGCACAGTACGGCAACCTCGTTGG
GGTCTCTATTGGTTACACCATCACTGCCTCCATAAGCTTAGTAGCGATTGGGAAAG
CAAATTGTTTTCATGGTAAGGGACATGGTGCGAAATGTACCGCATCGAATTATCCA
TACATGGGGGCATTTGGCGGCCTCCAGATTCTTCTAAGTCAGATTCCTAATTTTCA
CAAGCTATCTTTCCTCTCAATCATTGCCGCGGTTATGTCCTTCTCTTATGCATCTAT
TGGTATCGGTCTGGCCATCGCCAAAGTGGCAAGTGGGAAGGTTGGTAAGACAAC
ACTGACAGGTACGGTGATAGGAGTGGACGTATCTGCGTCTGATAAAGTGTGGAAA
GCGTTTCAAGCGGTTGGGGATATTGCGTTTTCGTACGCTTACACCACTATTCTCAT
TGAGATACAGGACACATTGAGATCAAGCCCACCAGAGAACAAAGTGATGAAGAAA
GCAAGTCTTATTGGAGTCTCAACCACAACTGTTTTCTACCTCTTATGTGGTTGCATT
GGGTATGCTGCATTCGGAAACTTATCCCCTGGTGACTTCCTTACCGACTTTGGGTT
TTACGAACCTTTCTGGCTCGTCATTTTCGCCAATGTTTGCATTGCTGTCCATTTAGT
AGGTGCCTATCAGGTATATGTTCAGCCTTTTTTCCAGTTTGTTGAGAGCAAATGTA
ACAAAAAGTGGCCTGAAAGCAATTTCATCAACAAAGAATACTCGTTGAAGATACCA
TTGCTCGGAAAATTTCGTGTCAACTTCTTCAGGCTGGTGTGGAGGACAAACTATGT
GATTTTGACAACATTTATTGCAATGATATTCCCCTTCTTCAACTCCATCTTGGGTTT

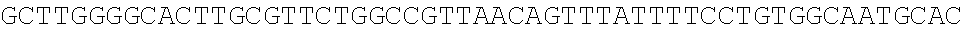

ATTGCTCAGACAAAGGTTAAGAAGTATTCGGGTAGATGGTTGGCGCTGAACCTCC
TCGTGCTGGTTTGCTTGATTGTCTCCGCCCTAGCTGCTGTGGGATCCATTGTTGG
CCTAATCAATAATGTCAAGAAATACAAGCCTTTCGAGAGTATAGACTAA
SEQ ID NO: 115: XP_013661681 AAP1 X1[Brassica napus] (protein)
MKSFNTDQHGHSAAESGDVYAMSDPTKNVDDDGREKRTGTWLTASAHIITAVIGSGV
LSLAWAIAQLGWIAGTLIIIFSFITYFTSTMLADCYRAPDPLTGKRNYTYMDVVRSYG
GRKVQLCGVAQYGNLIGITVGYTITASISLVAIGKANCYHNKGHHADCTISNYPYMAAF
GIIQILLSQIPNFHKLLSFLSLMAAVMSFAYASIGIGLAIATVAGGKVGKTNMTGTVVGVD
VTAAQKIWRSFQAVGDIAFAYAYATVLIEIQDTLRSSPAENKAMKRASFVGVSTTTFFYI
LCGCLGYAAFGNKAPGDFLTDFGFYEPFWLIDFANACIAFHLIGAYQVKPNPKGEKDC
FLFALSRSLANEKETYFLQVFAQPIFQFVEKKCNRNWPDNKFITSEYSVNIPFLGKFNIN

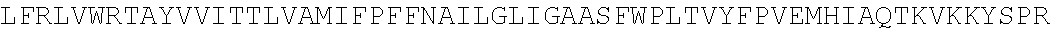

WIGLKMLCWVCLIVSLLAAAGSIAGLISSVKTYKPFRTIHE
SEQ ID NO: 116: XP_013661681 AAP1 X1[Brassica napus] (genomic)

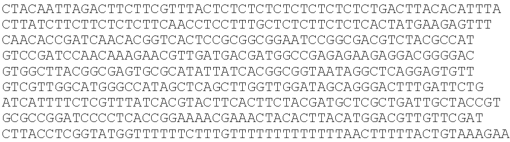

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGITT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

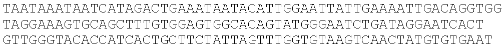

ATGTGATTTTACTTTATGACCGTTACCAAATGTACAGTTTAGTTATCTACCATATTG

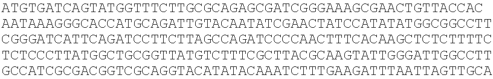

AAACAACAACTACTAACAATTATTGGTCATCAAGTTGTAGTTTTCAAAGTACCTATA
TCAATAATTGTAACAAGATAGATAATACAATAAAGTAACGGTTGATATGTTACGATA

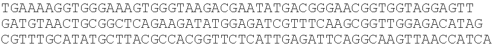

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

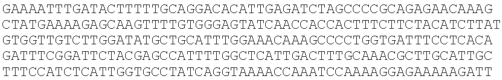

GTTTTCTTTTTGCACTTTCTCGCAGTTTAGCTAATGAAAAAGAAACATACTTCTTGC

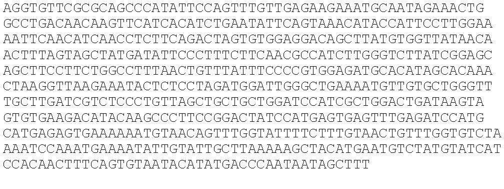

SEQ ID NO: 117: XP_013661682 AAP1 X2 [Brassica napus] (protein)
MKSFNTDQHGHSAAESGDVYAMSDPTKNVDDDGREKRTGTWLTASAHIITAVIGSGV
LSLAWAIAQLGWIAGTLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
VTAAQKIWRSFQAVGDIAFAYAYATVLIEIQDTLRSSPAENKAMKRASFVGVSTTTFFYI
LCGCLGYAAFGNKAPGDFLTDFGFYEPFWLIDFANACIAFHLIGAYQVFAQPIFQFVEK
KCNRNWPDNKFITSEYSVNIPFLGKFNINLFRLVWRTAYVVITTLVAMIFPFFNAILGLIG

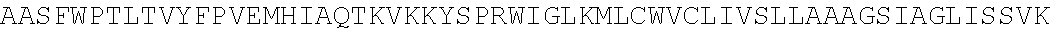

TYKPFRTIHE
SEQ ID NO: 118: XP_013661682 AAP1 X2 [Brassica napus] (genomic)

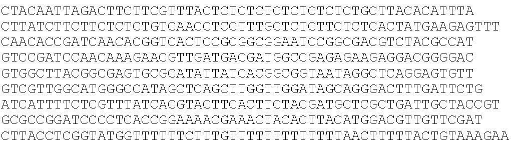

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCTT
TAATTTCCAACTATACAACGGTTGTTGAAATAATTAATTAGGCTGAAATTTTTCTTAA
ATAACTCACGGTTTTGTACTGTTTATAAGATCCAAAACTGTGCAACCCGAGGAAAA
CAGAAATTAAATAATAGTTTGACATATGAGGCAAAGGGTTTTGGTAGTATTAGCATT
ATTGTTGGTATTTGATGTTGATGGAGTATTATATATACTAGTATAGTTCCGTGGAGT
GTTTTATGTTTGATGTATGAAACAGAAGATTAATTAATAGCTAAAAACAAATGTAA

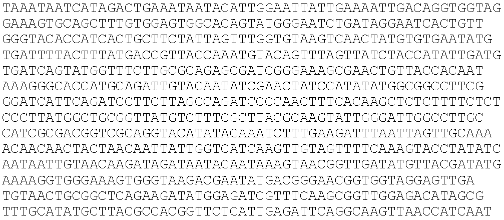

CAATTTTACTATTGAGTCTTGTTTGCTTTGAATATGAACCTCTTAATAGAATTGTGG
TTTGAAATTATTGACAATGACAATCAATTCTCTATGGACCACTTTTAATACAAAGGG
TAAAAAGAAGAGAATCTGTCTTTTAGCTTAAAGGTATAACATGTGCTTATTAGTGAC
AAGATGTCACATTCAAAGACAGCAAACAATGATATCAATGGACTTTAGCTTAATGA
GTTGACAATATAGTTAAAATTTTGTTGTCTCTTAATGATATTAGCGTTCACCTTTCTA
GTGTACATGCATTTAGTTCAATAGAGTGTATATGTCGACTAGAAAGTGACGGCTTA
ATAAGATTTAAGTTAAACACATGAGACAAAACTGGATTTGCACACACTAACCGGGA

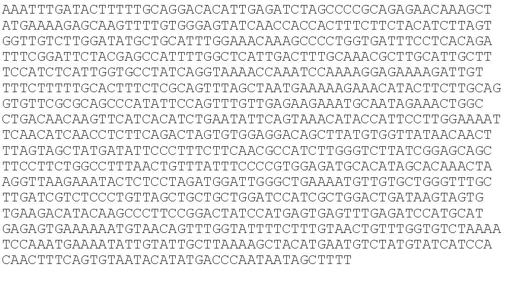

SEQ ID NO: 119: XP_013661683 AAP1 X3[Brassica napus] (protein)
MKSFNTDQHGHSAAESGDVYAMSDPTKNVDDDGREKRTGTWWLTASAHIITAVIGSGV
LSLAWAIAQLGWIAGTLLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
VTAAQKIWRSFQAVGDIAFAYAYATVLIEIQDTLRSSPAENKAMKRASFVGVSTTTFFYI
LCGCLGYAAFGNKAPGDFLTDFGFYEPFWLLIDFANACIAFHLIGAYQVFAQPIFQFVEK
KCNRNWPDNKFITSEYSVNIPFLLGKFNINLFRLVWRTAYVVITTLVAMIFPFFNAILGLIG

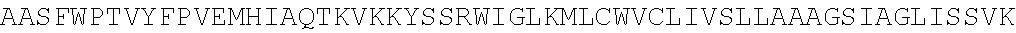

TYKPFRTIHE
SEQ ID NO: 120: XP_013661683 AAP1 X3[Brassica napus] (genomic):
Brara.I01660 | A09:11198108..11202102 forward

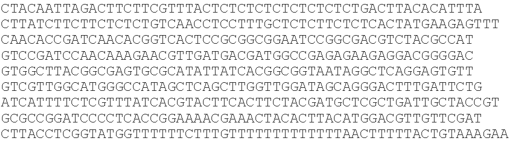

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

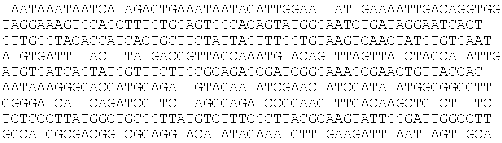

AAACAACAACTACTAACAATTATTGGTCATCAAGTTGTAGTTTTCAAAGTACCTATA
TCAATAATTGTAACAAGATAGATAATACAATAAAGTAACGGTTGATATGTTACGATA

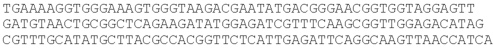

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

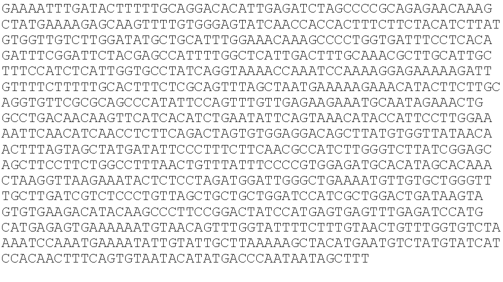

SEQ ID NO: 121: XP_013676681 AAP6 [Brassica napus] (protein)
MEKKSMFIEQSFTDHKSGDMNKNFDDDGRQKRTGTWMTGSAHIITAVIGSGVLSLAW
AIAQLGWVAGPAVLMAFSFITYFTSTMLADCYRSPDPVTGKRNYTYMEVVRSYLLGGR
KVMLCGLLAQYGNLIGITIGYTITASISMVAVKRSNCFHKNGHNVKCSTSNTPFMIIFACI
QIVLSQIPNFHNLSWLSILAAVMSFSYASIGIGLSIAKVAGGGVHARTALTGVTVGVDVT
GSDKVWRTFQAVGDIAFAYAYSTVLIEIQDTLKASPPSENKAMKRASLVFVSTTTFFY
MLCGCVGYAAFGNNAPGNFLTGFGFYEPFWLIDFANCIAVHLVGAYQVFCQPIFQFV
ESQSAKRWPDNKFITGEYKMNVPCGGDFGISLFRLVWRTSYVVVTAVVAMIFPFFND

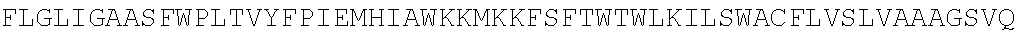

GLIQSLKDFKPFQAPE
SEQ ID NO: 122: XP_013676681 AAP6 [Brassica napus] (genomic): Brara.B01675
| A02:9628389..9631130 reverse

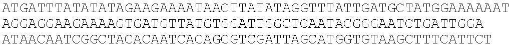

TTCTTTCAGAATGATAGTCAATAATAGCAGGCTCTCCTTTTTCACCTATTTTCACCC
ACGTTTATTATGTTAGGACAGGTGACTAACTCTTTTTTATAATTATTAATTTTACCTT
TTAAAGAACAGATGCTATGATAGGTAAGAGATATGATATATAATATCTACAAAAGTT
TTTCTTTGTCACAAGTTATTTGATATGTACAGAGTAATATAAATTTAAATTCTATTGA
GTGTGGGAGTCGAAAGGAGCTCAAATTTTCAAAGTGAAAAGTTAGATCTAGTAGG
ATCGTTGAGATTTTGTATTCTAAATTTATCAAATTTTTTTTGTCTGGAACTTTATATA
TTTATAATTATTAAGGAATGGGTTTTAAAGTACGAAAGAAAGAAAAAATTAAATG

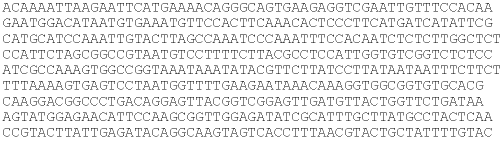

TCACCAGTTTAATTTAAAATGTTTTTTAAACATCTCGCACACCTGTTAAGAAAGGAG
TATTAGTTTTTCACTATAACCCTTATTAAATGTTTCAGCTAATACAAATGGTATCTTT
GGAAAAAATAATAATATACTCAGACCTGAATATACTACATATTTTTATAATTTAATAT
AACGGAAAATATGGTTATAATTGAAAGTTGAAACTTATGTTAAAACTTTGTATTGGC
AACTCTAAAACTAAACTCAGTTTTAAAAAATTAGCCATAAACTGACACTTCTGAAA
TGGAAGGATTATGTTTAGAGCTGAGTTTTAGAGACCGCGATAAGAGAGAGCCAAA
AAAAAATTAAACGTGTGTCGGTTTGTGACAAAGTAAAGGCCACTTCAGATGAATTA
TTATTTGTAGTAAAACATGAGAGGAAACCAGAGTCTAACTAGTAGGCTTTTATTCAA
TAAATAAATACTTATAAAATGATTTAACTTTGAGTACGGTTTACAACTGATGTTTTG
GCTTCTTTTGTACACAAAAGATTAATATTCTAACTTTAATTAATGTATTTCATTTTAA
AAGTAAAAAAAAATCTAAAAATATGGATTGTAGAATTTTATTGGAAAAAAACAAAAC
TAACAAAAACTAATTAATCAAAAATGACTCTTGACTTAGTTAATTTTATACTTTTATA
TTTATCACTAAATAACATTAAAGTCACCAATTACGTATTGTCATTTCAGATAATTGTA
AACGATTTAGTGAACTACATTTTGTGTGTGTTTTGATCTACCACTACTAAAGTATGT

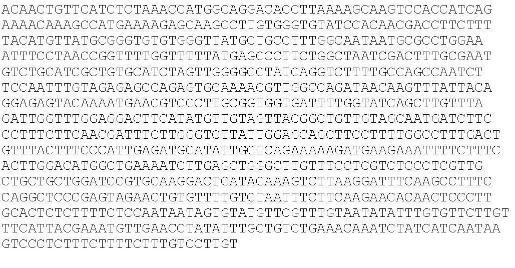

SEQ ID NO: 123: XP_013696427; XP_013640943; XP_013716098; AAP8
[Brassica napus] (protein)
MSPSSPPPTMKSLKTLHNPSAVESGNAAVKNVDDDGREKRTGTFLTASAHIITAVIGSG
VLSLAWALAQLGWVAGTMILVIFAIITYYTSTLLLADCYRAPDPITRTRNYTYMGVVRAYL
GGKKVQLCGLAQYGNLVGVSIGYTITASISLVAIGKANCFHGKGHGAKCTASNYPYMG
AFGGLQILLSQIPNFHKLSFLSIIAAVMSFSYASIGIGLAIAKVASGKVGKTTLTGTVIGVD
VSASDKVWKVFQAVGDIAFSYAYTTILIEIQDTLRSSPPENKVMKKASLIGVSTTTVFYL
LCGCIGYAAFGNIAPGDFLTDFGFYEPFWLVIFANVCIAVHLVGAYQVYVQPFFQFVES
KCNKKWPESNFINKEYSLKIPLLGKFRVNHFRLVWRTNYVILTTFIAMIFPFFNSILGLLG

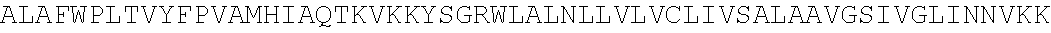

YKPFESID
SEQ ID NO: 124: XP_013696427; XP_013640943; XP_013716098; AAP8
[Brassica napus] (genomic)
ATGTCTCCCTCTCCCCCTCCTACAATGAAATCCTTGGACACACTCCACAATCCCTC
GGCGGTTGAGTCCGGTAACGCCGCTGTGAAGAACGTCGACGATGATGGTCGAGA
GAAGAGAACGGGGACGTTTCTGACGGCGAGTGCGCACATTATCACGGCGGTGAT
AGGCTCAGGAGTGTTGTCTTTGGCTTGGGCATTAGCACAGCTTGGTTGGGTGGCT
GGAACCATGATTTTGGTGATTTTCGCCATCATCACTTACTACACCTCTACTTTGCTC
GCCGATTGCTACAGAGCGCCGGACCCCATCACCAGAACACGCAACTACACGTAC
ATGGGCGTCGTTCGAGCTTACCTTGGTGGTAAAAAGGTGCAGCTATGTGGACTAG
CACAGTACGGCAACCTCGTTGGGGTCTCTATTGGTTACACCATCACTGCCTCCAT
AAGCTTAGTAGCGATTGGGAAAGCAAATTGTTTTCATGGTAAGGGACATGGTGCG
AAATGTACCGCATCGAATTATCCATACATGGGGGCATTTGGCGGCCTCCAGATTC
TTCTAAGTCAGATTCCTAATTTTCACAAGCTATCTTTCCTCTCAATCATTGCCGCG
TTATGTCCTTCTCTTATGCATCTATTGGTATCGGTCTGGCCATCGCCAAAGTGGCA
AGTGGGAAGGTTGGTAAGACAACACTGACAGGTACGGTGATAGGAGTGGACGTA
TCTGCGTCTGATAAAGTGTGGAAAGTGTTTCAAGCGGTTGGGGATATTGCGTTTTC
GTACGCTTACACCACTATTCTCATTGAGATCCAGGACACATTGAGATCAAGCCCAC
CAGAGAACAAAGTGATGAAGAAAGCAAGTCTTATTGGAGTCTCAACCACAACTGTT
TTCTACCTCTTATGTGGTTGCATTGGGTATGCTGCATTCGGAAACATAGCCCCTGGS
TGACTTCCTTACCGACTTTGGGTTTTACGAACCTTTCTGGCTCGTCATTTTCGCCA
ATGTTTGCATTGCTGTCCATTTAGTAGGTGCCTATCAGGTATACGTTCAGCCCTTT
TTCCAATTTGTTGAGAGCAAATGCAACAAAAAGTGGCCTGAAAGCAATTTCATCAA
CAAAGAGTACTCGTTGAAGATACCATTGCTCGGAAAATTTCGTGTCAACCACTTCA
GGCTGGTGTGGAGGACAAACTATGTGATTTTGACAACATTTATTGCAATGATATTC

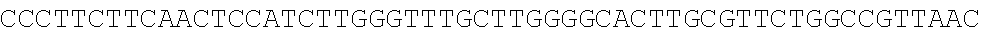

AGTTTATTTTCCTGTGGCAATGCACATTGCTCAGACAAAGGTTAAGAAGTATTCGG
GTAGATGGTTGGCGCTGAACCTCCTCGTGTTGGTTTGCTTGATTGTCTCCGCCTT
AGCGGCAGTGGGATCCATTGTTGGCCTAATCAATAATGTCAAGAAATACAAGCCTT
TCGAGAGTATAGACTAA
SEQ ID NO: 125: XP_013723586 AAP1-like X1 [Brassica napus] (protein)
MKSFNTDQHGHSAAESADVYAMSDPTKNVDDDGREKRTGTWLTASAHIITAVIGSGV
LSLAWAIAQLGWIAGTLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
VTAAQKIWRSFQAVGDIAFAYAYATVLIEIQDTLRSSPAENKAMKRASFVGVSTTTFFYI
LCGCLGYAAFGNKAPGDFLTDFGFYEPFWLIDFANACIAFHLIGAYQVKPNPKGEKDC
FLFALSRSLANEKETYFLQVFAQPIFQFVEKKCNRNWPDNKFITSEYSVNIPFLGKFSIN

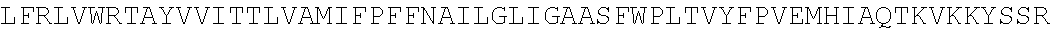

WIGLKMLCWVCLIVSLLAAAGSIAGLISSVKTYKPFRTIHE
SEQ ID NO: 126: XP_013723586 AAP1-like X1 [Brassica napus] (genomic):
Brara.I01660 | A09:11198108..11202102 forward

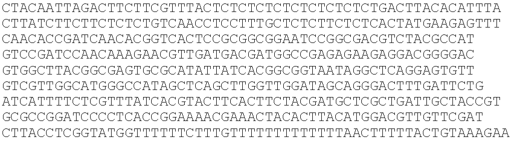

GATTGAAAAATAGACACTTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

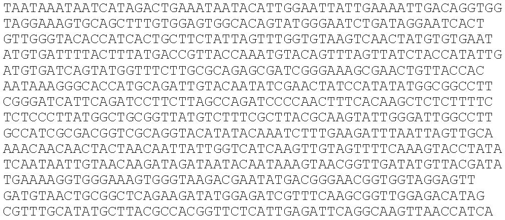

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

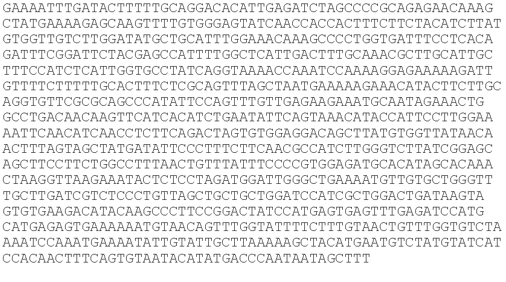

SEQ ID NO: 127: XP_013723587 AAP1-like isoform X2
[Brassica napus] (protein)
MKSFNTDQHGHSAAESADVYAMSDPTKNVDDDGREKRTGTWLTASAHIITAVIGSGV
LSLAWAIAQLGWIAGTLILIIFSFITYFTSTMLADCYRAPDPLTGKRNYTYMDVVRSYLG
GRKVQLCGVAQYGNLIGITVGYTITASISLVAIGKANCYHNKGHHADCTISNYPYMAAF
GIIQILLSQIPNFHKLSFLSLMAAVMSFAYASIGIGLAIATVAGGKVGKTNMTGTVVGVD
VTAAQKIWRSFQAVGDIAFAYAYATVLIEIQDTLRSSPAENKAMKRASFVGVSTTTFFYI
LCGCLGYAAFGNKAPGDFLTDFGFYEPFWLIDFANACIAFHLIGAYQVFAQPIFQFVEK
KCNRNWPDNKFITSEYSVNIPFLGKFSINLFRLVWRTAYVVITTLVAMIFPFFNAILGLIG

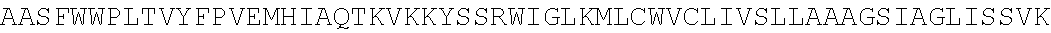

TYKPFRTIHE
SEQ ID NO: 128: XP_013723587 AAP1-like isoform X2 [Brassica napus]
(genomic): Brara.I01660 | A09:11198108..11202102 forward

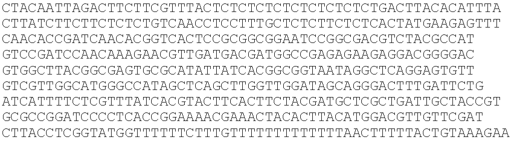

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAA

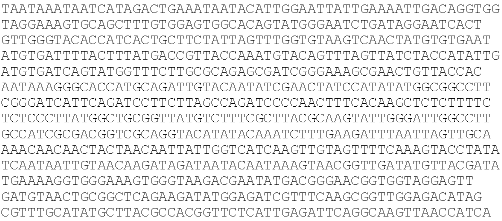

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

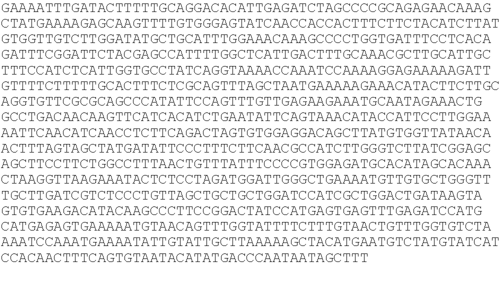

SEQ ID NO: 129: XP_013748815 AAP1-like [Brassica napus] (protein)
MKGFNTEQDHPAAESGNVYDVSDPTKNVDDDGREKRTGTWLTASAHIITAVIGSGVL
SLAWAIAQLGWIAGTLILVIFSFITYFTSTMLADCYRAPDPVTGKRNYTYMDVVRSYLG
GRKVQLCGVAQYGNLIGITVGYTITASSISLVAVGKANCFHKKGHEADCTISNYPYMAVF
GIIQIILSQIPNFHKLSFLSLMAAVMSFTYATIGIGLAIATVAGGKVGKTNMTGTVVGVDV
TAAQKIWRSFQAVGDIAFAYAYATVLIEIQDTLKSSPAENKSMKRASLVGVSTTTFFYIL
CGCLGYAAGFNKAPGDFLTDFGFYEPFWLIDFANACIAFHLIGAYQVFAQPIFQFVEKR
CNRNWWPDNKFITSEYSVNVPFLGKFNISLFRLVWRTAYVVITTVVAMIFPFFNAILGLIG

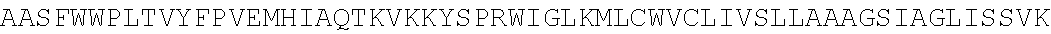

TYKPFRTIHE
SEQ ID NO: 130: XP_013748815 AAP1-like [Brassica napus] (genomic):
Brara.I01660 | A09:11198108..11202102 forward

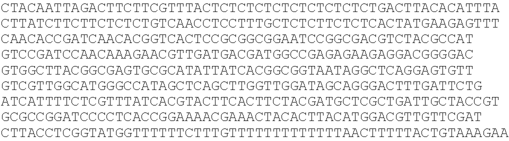

GATTGAAAAATAGACACTTTTTAGACCAACCCTTTTTTGAAAAGGGTTCTATACCAA
AAATTCAATTTTGTTAAGTAATTTTTAAGGCATGGTTGGTTCCTTTTCCAAAAAAAAA
GAGAGAGGATAACATAAGGTTCTCTTTCATTAAAATAATATACTGATTTTGTCTGGT
ATTTAGTTAGGCTGATCCTTGTCTGGAAAACTATTGTGGTCAGAATAGGAAAATAA
TTATTAGTATTAAAAATTTATACTAATATTAATTAAAAATGACAAAATATATATTAGTA
ATAAAAACATATAATATAATTTATAAGCGACAGTAACTTTTAGGTCAATTTGAAATAA
AACTGGTTATGTAATGAGTTTATATAGAACAATGATGGTGGTTTATAAAATAGTTGA
TGTTACGAACTATAAGATCAATCATAAGAAATCATCATTGATATCTTTTGAAACAAT
CGAAAAGCTTATGCATCCGATGAGTTGTGGTTAGGAATGTAGATAAAGTAATGGAT
TTATAGATTTTCTAACATTTCCTGCCAATTTGGTTTTACAGAAGAAAAAAAATCTATC
GGGCATATAAATTACTGTTGCGTATAAATTGATTTTTTTTGTAGACGCTTATAAATT
GATATATATCCTTTTAAATATTTAAATTTAACTGAATATAAAAACAGTGGTAACCGTT
CTTTTAATTTTCTAGTTAGAAAAAATGTTGTTGAAATAATTAAAGGCTTGGTTATTAT
TTATACATGGATTATATTCGTAAGCAATTTAAAGTTTGTTGTCTCTAGTTTAATAAAC
GATGATTTTTTACGAATTCTTTTTATTAAGTAAAAACACTGAGTTATTGACAAAAAAA
AAGAGTAAAACAACTGAGTTCAATGTAAAGTGTGGTAACCGTCCTTTTAATTTTCTA
ATGATAAACTATGGTTGTTGAAAAATTAATTAGACTTCGCTATTATTTTCATAAGGA
TTACATACTTCAAATAATTTAGAGTATGTTGTCTAGTTCAATAAACAATGAGGTTTTA
CTTCAAATCATTTTAAGTAAAACATTGAACTGAACGCAAAAGTGTGGTAACCGTCC
TTTAATTTCCAACTATACAACGGTTGTTGAAATAATTAATTAGGCTGAAATTTTTCTT
AAATAACTCACGGTTTTGTACTGTTTATAAGATCCAAAACTGTGCAACCCGAGGAA
AACAGAAATTAAATAATAGTTTGACATATGAGGCAAAGGGTTTTGGTAGTATTAGC
ATTATTGTTGGTATTTGATGTTGATGGAGTATTATATATACTAGTATAGTTCCGTGG
AGTGTTTTATGTTTGATGTATGAAACAGAAGATTAATTAATAGCTTAAAAACAAATG

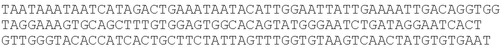

ATGTGATTTTACTTTATGACCGTTACCAAATGTACAGTTTAGTTATCTACCATATTG

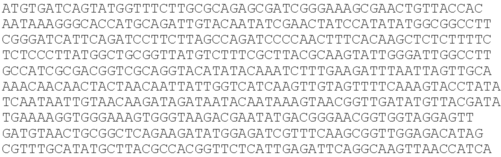

ATCAATTTTACTATTGAGTCTTGTTTGCTTTGAATATGAACCTCTTAATAGAATTGTG
GTTTGAAATTATTGACAATGACAATCAATTCTCTATGGACCACTTTTAATACAAAGG
GTAAAAAGAAGAGAATCTGTCTTTTAGCTTAAAGGTATAACATGTGCTTATTAGTGA
CAAGATGTCACATTCAAAGACAGCAAACAATGATATCAATGGACTTTAGCTTAATG
AGTTGACAATATAGTTAAAATTTTGTTGTCTCTTAATGATATTAGCGTTCACCTTTCT
AGTGTACATGCATTTAGTTCAATAGAGTGTATATGTCGACTAGAAAGTGACGGCTT
AATAAGATTTAAGTTAAACACATGAGACAAAACTGGATTTGCACACACTAACCGGT

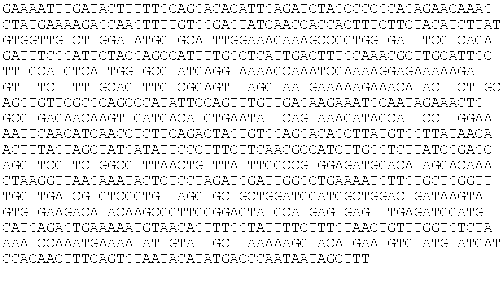

BARLEY
SEQ ID NO: 131: BAJ85485 [Hordeum vulgare subsp. vulgare] (protein)
MGMEKSKANPAAFSIAEAGFGDRTDIDDDGRERRTGTLVTASAHIITAVIGSGVLSLA
WAIAQLGWVIGPAVLVVAFSVITWWFCSSLLADCYRSPDPVHGKRNYTYGQAVRANLGV
SKYRLCSLAQYLNLVGVTIGYTITTAISMGAIGRSNCFHRNGHNAACEASNTTNMIIFAA
IQILLSQLPNFHKVWWLSIVAAVMSLAYSSIGLGLSIAKIAGGVHAKTTLTGVTVGVDVS
ASEKIWWRTFQSLGDIAFAYSYSNVLIEIQDTLRSSPAENTVMKKASLIGVSTTTTFYMLC
GVLGYAAFGSSAPGNFLTGFGFYEPFWLVDVGNVCIVVHLVGAYQVFCQPFYQFVEG
WARSRWPDSAFLHAERVVQLPAIVGGGEFPVSPFRLVWRTAYVALTAVVAMLFPFFN

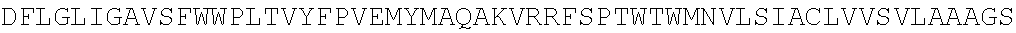

VQGLVKDVAGYKPFKVS
SEQ ID NO: 132: BAJ85485 [Hordeum vulgare subsp. vulgare] (genomic)
ATGGGGATGGAGAAGAGCAAGGCTAACCCTGCCGCCTTCAGCATCGCTGAGGCC
GGCTTTGGAGACCGGACGGACATCGACGACGACGGCCCGCGAGAGGCGTACCGG
TACGCTGGTGACGGCCAGCGCACACATCATCACGGCGGTGATCGGGTCCGGGGT
GCTGTCGCTGGCGTGGGCCATCGCACAGCTCGGGTGGGTCATCGGCCCCGCGCG
TGCTCGTCGCCTTCTCCGTCATCACCTGGTTCTGCTCCAGCCTACTGGCCGACTG
CTATCGCTCGCCGGACCCCGTCCACGGCAAGCGCAACTACACCTACGGCCAAGC
CGTCAGGGCAAACCTAGGAGTTAGCAAGTACAGGCTCTGCTCACTGGCCCAATAC
CTCAACTTGGTTGGCGTGACCATTGGCTACACCATCACCACGGCCATCAGCATGG
GGGCGATCGGACGGTCCAATTGCTTCCACCGCAATGGCCACAATGCGGCCTGCG
AGGCATCCAACACCACCAACATGATTATATTTGCTGCCATCCAAATCTTGCTCTCG
CAGCTCCCCAACTTCCACAAGGTCTGGTGGCTCTCCATTGTTGCTGCCGTCATGT
CCCTCGCCTACTCGTCCATTGGTCTCGGCCTCTCCATAGCAAAAATCGCAGGTGG
GGTGCATGCCAAGACAACGCTAACAGGGGTGACCGTTGGGGTGGATGTATCTGC
GAGTGAGAAAATTTGGAGAACGTTCCAGTCTCTTGGGGACATCGCCTTTGCATAC
TCCTACTCCAATGTTCTCATCGAAATCCAGGACACGCTGCGGTCGAGCCCGGCG
GAGAACACGGTGATGAAGAAGGCATCCTTGATCGGCGTTTCCACGACCACCACGT
TCTACATGCTGTGCGGGGTGCTGGGCTACGCGGCGTTCGGCAGCAGCGCCCCG
GGTAACTTCCTCACGGGCTTCGGCTTCTACGAGCCCTTCTGGCTCGTCGACGTCG
GCAACGTCTGCATCGTCGTGCACCTCGTCGGCGCCTACCAGGTCTTCTGCCAGC
CCTTCTACCAGTTCGTCGAGGGCTGGGCGCGCTCCCGGTGGCCCGACAGCGCCT
TCCTCCACGCCGAGCGAGTCGTGCAACTCCCGGCCATTGTCGGCGGCGGCGAGT
TCCCCGTGAGCCCATTTCGCCTGGTCTGGCGAACGGCGTACGTGGCCCTCACGG
CGGTGGTGGCCATGTTGTTCCCCTTCTTCAACGACTTTCTTGGCCTCATCGGCCGC

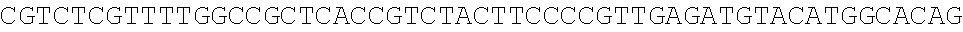

GCCAAGGTGCGGCGGTTCTCGCCGACGTGGACGTGGATGAACGTGCTTAGCATC
GCCTGCCTTGTCGTCTCTGTCCTCGCAGCCGCTGGTTCGGTGCAGGGGCTCGTC
AAGGACGTGGCAGGGTACAAGCCATTCAAGGTCTCCTAA
SEQ ID NO: 133: BAJ91439.1 predicted protein [Hordeum
vulgare subsp. vulgare] (protein)
MTKDVEMAARNGSKGAAAGEAYYPSPPGQGGDVDVDDDGKQRRTGTVWTASAHIIT
AVIGSGVLSLAWATAQLGWVVGPVTLMLFAAITYYTSGLLADCYRTGDPLTGKRNYTY
MDAVASYLSRWQVWACGVFQYVNLVGTAIGYTITASISAAAINKANCFHKNGRAADC
GVYDSMYMVVFGVVQIFFSQVPNFHDLWWWLSILAAVMSFTYASIAVGLSLAQTISGPT
GKSTLTGTEVGVDVDSAQKIWLAFQALGDIAFAYSYSMILIEIQDTVRSPPAENKTMKK
ATLVGVSTTTAFYMLCGCLGYAAFGNGAKGNILTGFGFYEPYWLIDFANVCIVVHLVG
AYQVFCQPIFAAVENFAAATWWPNAGFITREHRVAAGKRLGFNLNLFRLTWRTAFVMV

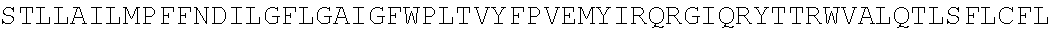

VSLAAAVASIEGVTESLKNYVPFKTKS
SEQ ID NO: 134: BAJ1439.1 predicted protein [Hordeum
vulgare subsp. vulgare] (genomic)
ATGGGGGAGAACGGTGTGGTGGCGAGCAAGCTGTGCTACCCGGCGGCGGCCAT
GGAGGTGGTCGCCGCCGAGCTCGGCCACACGGCCGGCTCCAAGCTGTACGACG
ACGACGGCCGCCTCAAGCGCACCGGGACGATGTGGACGGCGAGCGCGCACATC
ATCACGGCGGTGATCGGCTCCGGCGTGCTGTCGCTGGGGTGGGCGATCGCGCA
GCTGGGTTGGGTGGCCGGCCCCGCCGTCATGCTGCTCTTCTCGTTCGTCACCTA
CTACACCTCCGCGCTGCTCGCCGACTGCTACCGCTCCGGCGACGAGAGCACCGG
CAAGCGCAACTACACCTACATGGACGCCGTGAACGCCAACCTGAGTGGCATCAA
GGTCCAGGTCTGCGGGTTCCTGCAGTACGCCAACATCGTCGGCGTCGCCATCGG
CTACACCATTGCCGCCTCCATTAGCATGCTGGCGATCAAGCGGGCGAACTGCTTC
CACGTCGAGGGGCACGGCGACCCGTGCAACATCTCGAGCACGCCGTACATGATC
ATCTTCGGCGTGGCGGAGATCTTCTTCTCGCAGATCCCGGACTTCGACCAGATCT
CGTGGCTGTCCATCCTCGCCGCCGTCATGTCGTTCACCTACTCCACCATCGGGCT
CGGCCTCGGCGTCGTGCAGGTGGTGGCCAACGGCGGCGTCAAGGGGAGCCTCA
CCGGGATCAGCATCGGCGTGGTGACGCCCATGGACAAGGTGTGGCGGAGCCTG
CAGGCGTTCGGCGACATCGCCTTCGCCTACTCCTACTCCCTCATCCTCATCGAGA
TCCAGGACACCATCCGGGCGCCGCCGCCGTCGGAGTCGAGGGTGATGCGGCGC
GCCACCGTGGTGAGCGTCGCCGTCACCACGCTCTTCTACATGCTCTGCGGCTGC
ACGGGGTACGCGGCGTTCGGCGACGCCGCGCCGGGCAACCTCCTCACCGGGTT
CGGCTTCTACGAGCCCTTCTGGCTCCTCGACGTTGCCAACGCCGCCATCGTCGT
CCACCTCGTCGGCGCCTACCAGGTCTACTGCCAGCCGCTGTTCGCCTTCGTCGA
GAAGTGGGCGCAGCAGCGGTGGCCGAAATCATGGTACATCACCAAGGATATCGA
CGTGCCGCTCTCCCTCTCCGGCGGCGGCGGCGGCGGCGGAAGGTGCTACAAGC
TGAACCTGTTCAGGCTGACATGGAGGTCGGCGTTCGTGGTGGCGACGACGGTGG

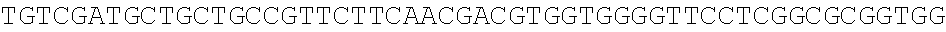

GGTTCTGGCCGCTCACCGTCTACTTCCCGGTGGAGATGTACATCGTGCAGAAGA
GGATACCGAGGTGGAGCACGCGGTGGGTGTGCCTGCAGCTGCTCAGCCTCGCC
TGCCTCGCCATCACCGTCGCCTCCGCCGCCGGCTCCATCGCCGGAATCCTCTCC
GACCTCAAGGTCTACAAGCCGTTCGCCACCACCTACTAA
WHEAT
SEQ ID NO: 135: EMS56484 [Triticum urartu] (protein)
MEVVTALTNVEVPATGTVAEATDRSDAERASKWARCWRILGWWTLGEGIVGEDFGWS
WGGGAGGCFYFPYFTCGQGSGDDDCVRGGAWGRGFGAGASPMTTAFHSAARGG
AGGGLGQVAPAILSPDMPVALGLGVGHLSEGHGSPQPPAPVTLVDPLRDSARGFTRE
EVVAFGGIPDPVSAGRWMSARIQELPEVDDMQQRCAMREAKLHDAEISTGYFSSHG
SDPFVVATHSDGGQRAFGYWIYPLGDASQLEAMGMEKGKADPATFSIAEAGFGDRT
DIDDDGRERRTGTLVTASAHIITAVIGSGVLSLAQAIAQLGWVIGPAVLVAFSVITWFCS
SLLADCYRSPDPVHGKRNYTYGQAVRANLGVSKYRLCSLAQYVNLVGVTIGYTITTAI
SMGAIGRSNCFHRNGHNAACEASNTTNMIIFAAIQILLSQLPNFHKIWWLSIVAAVMSL
AYSSIGLGLSIAKIAGGVHAKTALTGVTVGVDVSASEKIWRTFQSLGDIAFAYSYSNVLI
EIQDTLRSSPAENKVMKKASLIGVSTTTTFYMLCGVLGYAAFGSSAPGNFLTGFGFYE
PFWLVDIGNVCIIVHLVGAYQVFCQPIYQFVEGWARSRWPDSAFLHAERVLRLPAVLG

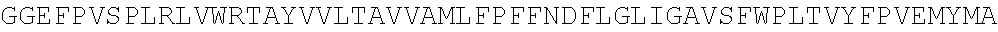

QAKVRRFSPTWTWMNVLSVACLVVSVLAAAGSVQGLIKDVAGYKPFKVS
SEQ ID NO: 136: EMS56484 [Triticum urartu] (genomic)
ATGGAGGTGGTGACGGCCTTGACCAATGTTGAGGTTCCTGCGACTGGGACTGTG
GCTGAGGCTACCGACAGGTCTGATGCTGAGAGGGCGTCCAAGTGGGCGCGGTG
CTGGCGGATCCTTGGCTGGACGCTTGGTGAGGGCATCGTCGGCGAGGACTTTGG
ATGGAGTTGGGGAGGTGGAGCTGGTGGTTGCTTCTATTTCCCTTACTTCACATGT
GGTCAAGGCTCCGGAGATGATGACTGCGTCCGAGGTGGGGCTTCCCCCAGGGG
GTTCGGGGCAGGTGCGTCTCCTATGACGACGGCGTTCCACTCTGCTGCGAGGGG
TGGGGCGGGAGGAGGGCTCGGGCAGGTGGCCCCCGCCATCCTCTCTCCCGACA
TGCCCGTGGCCCTGGGCCTCGGTGTGGGGCACTTGTCCGAGGGGCATGGGAGC
CCGCAGCCGCCTGCTCCGGTAACCTTGGTTGACCCTTTGCGGGATTCAGCGCGA
GGCTTTACTAGGGAGGAGGTCGTTGCTTTTGGCGGGATTCCTGACCCGGTCTCG
GCGGGGAGATGGATGAGTGCTCGCATTCAGGAGCTTCCGGAGGTTGATGACATG
CAGCAGAGGTGCGCTATGAGGGAGGCCAAGCTTCATGATGCTGAGATCTCTACT
GGTTATTTTTCGAGCCACGGCAGTGATCCGTTCGTGGTCGCTACTCACTCCGATG
GAGGCCAGAGAGCATTTGGTTACTGGATCTATCCGCTGGGAGACGCTAGCCAGC
TAGAAGCAATGGGGATGGAGAAGGGCAAGGCTGACCCTGCCACCTTCAGCATCG
CTGAGGCCGGCTTTGGAGACCGGACGGACATCGACGACGACGGACGCGAGAGG
CGTACCGGTACGCTGGTGACGGCGAGCGCCCACATCATCACGGCGGTCATCGG
GTCCGGGGGTGCTGTCGCTGGCGTGGGCCATCGCGCAGCTCGGGTGGGTCATCG
GCCCCGCCGTGCTCGTCGCCTTCTCCGTCATCACCTGGTTCTGCTCCAGCCTACT
GGCCGACTGCTACCGCTCACCGGACCCCGTCCACGGCAAGCGCAACTACACCTA
CGGCCAGGCCGTCAGGGCCAACCTAGGAGTTAGCAAATACAGGCTCTGCTCTCT
GGCCCAATACGTCAACTTGGTTGGCGTCACCATTGGCTACACCATCACCACGGCC
ATCAGCATGGGGGGCGATCGGACGGTCGAATTGCTTCCACCGCAATGGCCACAAT
GCGGCCTGCGAGGCATCCAACACCACCAACATGATTATATTTGCTGCCATCCAAA
TCTTGCTCTCGCAACTCCCCAACTTCCACAAGATCTGGTGGCTCTCCATTGTTGCC
GCCGTCATGTCCCTCGCCTACTCCTCCATTGGTCTCGGCCTCTCCATAGCAAAAA
TCGCAGGTGGGGTGCATGCCAAGACAGCGCTAACAGGGGTGACCGTTGGGGTG
GATGTATCCGCGAGTGAGAAAATTTGGAGGACGTTCCAGTCTCTTGGGGACATCG
CCTTTGCATACTCCTACTCCAATGTGCTCATCGAAATCCAGGACACGCTGCGGTC
GAGCCCGGCAGAGAACAAGGTGATGAAGAAGGCGTCCTTGATCGGTGTTTCCAC
GACCACCACGTTCTACATGCTGTGCGGGGTGCTGGGCTACGCGGCGTTCGGCAG
CAGCGCCCCCGGGTAACTTCCTCACGGGCTTCGGCTTCTACGAGCCCTTCTGGCT
CGTCGACATCGGCAACGTTTGCATCATCGTGCACCTCGTTGGCGCCTACCAGGTC

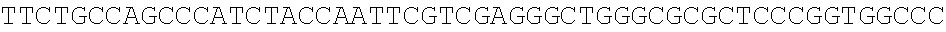

GACAGCGCCTTCCTCCATGCCGAGCGCGTGTTGCGCCTTCCGGCCGTTCTCGGA
GGCGGAGAGTTCCCGGTTAGCCCGTTACGCCTGGTCTGGCGAACGGCGTACGTG
GTTCTCACGGCGGTGGTGGCCATGCTGTTCCCCTTCTTCAACGACTTCCTTGGCC
TCATTGGCGCCGTCTCGTTTTGGCCGCTCACCGTCTACTTCCCCGTTGAGATGTA
CATGGCACAAGCCAAAGTGCGCCGGTTCTCGCCGACGTGGACGTGGATGAACGT
GCTTAGCGTCGCGTGCCTTGTCGTCTCTGTCCTCGCCGCAGCTGGTTCTGTGCAG
GGGCTCATCAAGGACGTCGCAGGGTACAAGCCATTCAAGGTCTCCTAA
SEQ ID NO: 137: EMS68703.1 TRIUR3_33957 [Triticum urartu] (protein)
MGVLGLIQLVGRRRGEYPLVRDTVTPQGGGESGGGGGGAMDVDGHLPRTHGDVDD
DGRERRTGTVWTAAAHIITAVIGSGVLSLAWAMAQLGWVAGPLTLVLFAIITFYTCGLL
ADRYRVGDPVTGKRNYTYTEAVQAYLGTCSPQARPFLLIKMQPEMMCMCSGGWHV
WFCGFCQYVNMFGTGIGYTITASTSAAALKKSNCFHWHGHKADCSQYLSAYIIAFGVV
QVIFCQVPNFHKLSWLSIVAAIMSFSYATIAVGLSLAQTISGPRGRTSLTGTEVGVDVD
ASQKVWMTFQALGNVAFAYSYSIILIEIQDTLRSPPGENKTMRKATLMGISTTTAFYML
CGCLGYSAFGNDANGNILTGFGFYEPYWLVDFANVCIVLHLVGGFQVFCQPLFAAMYI

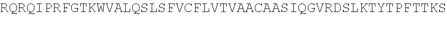

SEQ ID NO: 138: EMS68703.1 TRIUR3_33957 [Triticum urartu] (genomic)
ATGGGGGTCCTCGGCCTGATCCAACTGGTCGGGAGACGACGTGGTGAGTACCCC
CTAGTCCGGGACACCGTCACCCCCCAGGGAGGCGGCGAGAGCGGCGGCGGCG
GAGGCGGGGCCATGGACGTCGACGGCCACCTTCCCCGCACCCACGGCGACGTC
GACGACGACGGCAGGGAGAGGAGAACAGGGACGGTATGGACGGCGGCGGCG
CACATCATAACGGCGGTGATCGGGTCCGGCGTGCTGTCGCTGGCCTGGGCCATG
GCGCAGCTGGGCTGGGTGGCCGGGCCGCTCACCCTGGTGCTCTTCGCCATCATC
ACCTTCTACACCTGCGGCCTCCTCGCCGACCGCTACCGCGTCGGCGACCCCGTC
ACGGGCAAGCGCAACTACACCTACACCGAGGCCGTCCAGGCCTACCTAGGTACG
TGCTCGCCTCAAGCTCGCCCGTTTTTACTCATCAAAATGCAACCTGAGATGATGTG
CATGTGTTCAGGCGGGTGGCACGTCTGGTTCTGCGGCTTCTGCCAGTACGTCAA
CATGTTCGGCACCGGCATCGGCTACACCATCACCGCCTCCACCAGCGCCGCGGC
CTTGAAGAAGTCCAACTGCTTCCACTGGCACGGGCACAAGGCGGACTGCAGCCA
GTACCTGAGCGCCTACATCATCGCCTTCGGGGTGGTGCAGGTCATCTTCTGCCAG
GTGCCCAACTTCCACAAGCTCTCGTGGCTCTCCATCGTCGCCGCCATCATGTCCT
TCTCCTACGCCACCATCGCCGTCGGCCTCTCGCTGGCGCAGACCATCTCGGGGGC
CCAGGGGGAGGACGTCGCTGACCGGCACGGAGGTCGGGGTGGACGTCGACGC
CTCGCAGAAGGTCTGGATGACGTTCCAGGCCCTCGGCAACGTCGCCTTCGCCTA
CTCCTACTCCATAATCCTCATCGAGATCCAGGACACGCTGCGGTCACCTCCGGGC
GAGAACAAGACGATGCGGAAGGCGACGCTGATGGGCATCTCGACGACGACGGC
CTTCTACATGCTGTGCGGCTGCCTGGGCTACTCGGCCTTCGGCAACGACGCCAA
CGGCAACATCCTGACGGGGTTCGGCTTCTACGAGCCCTACTGGCTGGTGGACTT
CGCCAACGTCTGCATCGTGCTCCACCTGGTGGGCGGCTTCCAGGTCTTCTGCCA
GCCGCTGTTCGCGGCGATGTACATCCGGCAGCGGCAGATCCCGCGGTTCGGCA
CCAAGTGGGTGGCGCTGCAGAGCCTCAGCTTCGTCTGCTTCCTCGTCACCGTCG
CCGCCTGCGCCGCCTCCATCCAGGGCGTCCGCGACTCGCTCAAGACCTACACGC

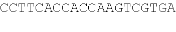

BRASSICA RAPA
SEQ ID NO: 139: VDC65345.1 unnamed protein product
[Brassica rapa] (protein)
MSPSPPLTMKSLDTLHNPSAVESGNAAVKNVDDDGREKRTGTFTASAHIITAVIGSG
VLSLAWALAQLGWVAGTMILVIFAIITYYTSTLLADCYRAPDPITGTRNYTYMGVVRAYL
GGKKVQLCGLAQYGNLVGVSIGYTITASISLVAIGRANCFHDKGHGAKCTASNYPYMV
AFGGLQILLSQIPNFHKLSFLSIIAAVMSFFSYASIGIGLAIAKVASGKVGKTTLTGTVIGVD
VSASDKVWKAFQAVGDIAFSYAYTTILIEIQDTLRSSPPENKVMKKASLIGVSTTTVFYL
LCGCIGYAAFGNLSPGDFLTDFGFYEPFWLVIFANVCIAVHLVGAYQVYVQPFFQFVE
SKCNKKWPESNFINKEYSLLKIPLLGKFRVNFFRLVWRTNYVILTTFIAMIFPFFNSILGLL

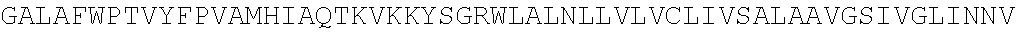

KKYKPFESID
SEQ ID NO: 140: VDCT65345.1 unnamed protein product
[Brassica rapa] (genomic)
ATGTCTCCTTCTCCCCCTCTTACAATGAAATCCTTGGACACACTCCACAATCCCTCG
GCGGTTGAGTCCGGTAACGCCGCTGTGAAGAACGTCGACGATGATGGTCGAGAG
AAGAGAACGGGGACGTTTCTGACGGCGAGTGCGCACATTATCACGGCGGTGATAG
GCTCAGGAGTGTTGTCTTTGGCTTGGGCATTAGCACAGCTTGGTTGGGTGGCTGG
AACCATGATTTTGGTGATTTTCGCCATCATCACTTACTACACGTCTACTTTGCTCGC
CGATTGCTACAGAGCGCCGGACCCCATCACCGGAACACGCAACTACACGTACATG
GGCGTCGTTCGAGCTTACCTTGGTGGTAAAAAGGTGCAGCTATGTGGACTAGCAC
AGTACGGAAACCTCGTTGGGGTCTCTATTGGTTACACCATCACTGCCTCCATAAGC
TTAGTAGCGATTGGGAGAGCAAATTGTTTTCATGACAAGGGACATGGTGCGAAATG
TACCGCATCGAATTATCCATACATGGTGGCATTTGGCGGCCTCCAGATTCTTCTAAG
TCAGATTCCTAATTTTCACAAGCTATCTTTCCTCTCAATCATTGCCGCGGTTATGTCC
TTCTCTTATGCATCTATTGGTATCGGTCTGGCCATCGCCAAAGTAGCAAGTGGGAA
GGTTGGTAAGACAACACTGACAGGTACGGTGATAGGAGTGGACGTATCTGCGTCT
GATAAAGTGTGGAAAGCGTTTCAAGCGGTTGGGGATATTGCGTTTTCGTACGCTTA
CACCACTATTCTCATTGAGATACAGGACACATTGAGATCAAGCCCACCAGAGAACA
AAGTGATGAAGAAAGCAAGTCTTATTGGAGTCTCAACCACAACTGTTTTCTACCTCT
TATGTGGTTGCATTGGATATGCTGCATTCGGAAACTTATCCCCTGGTGACTTCCTA
CCGACTTTGGGTTTTACGAACCTTTCTGGCTCGTCATTTTCGCCAATGTTTGCATTG
CTGTCCATTTAGTAGGTGCCTATCAGGTATATGTTCAGCCTTTTTTCCAGTTTGTTGA
GAGCAAATGTAACAAAAAGTGGCCTGAAAGCAATTTCATCAACAAAGAATACTCGTT
GAAGATACCATTGCTCGGAAAATTTCGTGTCAACTTCTTCAGGCTGGTGTGGAGGA
CAAACTATGTGATTTTGACAACATTTATTGCAATGATATTCCCCTTCTTCAACTCCAT

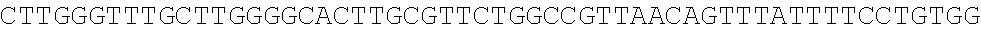

CAATGCACATTGCTCAGACAAAGGTTAAGAAGTATTCGGGTAGATGGTTGGCGCTG
AACCTCCTCGTGCTGGTTTGCTTGATTGTCTCCGCCCTAGCTGCTGTGGGATCCAT
TGTTGGCCTAATCAATAATGTCAAGAAATACAAGCCTTTCGAGAGTATAGACTAA
SEQ ID NO: 141: RID57273.1 hypothetical protein BRARA_F00660
[Brassica rapa] (protein)
MSPSPPLTMKSLDTLHNPSAVESGNAAVKNVDDDGREKRTGTFLTASAHIITAVIGSG
VLSLAWALAQLGWVAGTMILVIFAIITYYTSTLLADCYRAPDPITGTRNYTYMGVVRAYL
GGKKVQLCGLAQYGNLVGVSIGYTITASISLVAIGRANCFHDKGHGAKCTASNYPYMV
AFGGLQILLSQIPNFHKLSFLSIIAAVMSFSYASIGIGLAIAKVASGKVGKTTLTGTVIGVD
VSASDKVWKAFQAVGDIAFSYSYTTILIEIQDTLRSSPPENKVMKKASLIGVSTTTVFYL
LCGCIGYAAFGNLSPGDFLTDFGFYEPFWLVIFANVCIAVHLVGAYQVYVQPFFQFVE
SKCNKKWPESNFINKEYSLKIPLLGKFRVNFFRLVWRTNYVILTTFIAMIFPFFNSILGLL

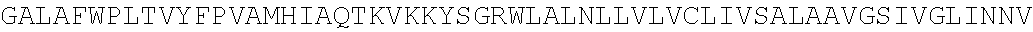

KKYKPFESID
SEQ ID NO: 142: RID57273.1 hypothetical protein BRARA_F00660
[Brassica rapa] (genomic)
ATGAAATCCTTGGACACACTCCACAATCCCTCGGCGGTTGAGTCCGGTAACGCCG
CTGTGAAGAACGTCGACGATGATGGTCGAGAGAAGAGAACGGGGACGTTTCTGA
CGGCGAGTGCGCACATTATCACGGCGGTGATAGGCTCAGGAGTGTTGTCTITTGG
CTTGGGCATTAGCACAGCTTGGTTGGGTGGCTGGAACCATGATTTTGGTGATTTTT
CGCCATCATCACTTACTACACGTCTACTTTGCTCGCCGATTGCTACAGAGCGCCG
GACCCCATCACCGGAACACGCAACTACACGTACATGGGCGTCGTTCGAGCTTACC
TTGGTGGTAAAAAGGTGCAGCTATGTGGACTAGCACAGTACGGAAACCTCGTTGG
GGTCTCTATTGGTTACACCATCACTGCCTCCATAAGCTTAGTAGCGATTGGGAGA
GCAAATTGTTTTCATGACAAGGGACATGGTGCGAAATGTACCGCATCGAATTATCC
ATACATGGTGGCATTTGGCGGCCTCCAGATTCTTCTAAGTCAGATTCCTAATTTTC
ACAAGCTATCTTTCCTCTCAATCATTGCCGCGGTTATGTCCTTCTCTTATGCATCTA
TTGGTATCGGTCTGGCCATCGCCAAAGTGGCAAGTGGGAAGGTTGGTAAGACAA
CACTGACAGGTACGGTGATAGGAGTGGACGTATCTGCGTCTGATAAAGTGTGGAA
AGCGTTTCAAGCGGTTGGGGATATTGCGTTTTCGTACGCTTACACCACTATTCT
CATTGAGATACAGGACACATTGAGATCAAGCCCACCAGAGAACAAAGTGATGAAG
AAAGCAAGTCTTATTGGAGTCTCAACCACAACTGTTTTCTACCTCTTATGTGGTTG
CATTGGGTATGCTGCATTCGGAAACTTATCCCCTGGTGACTTCCTTACCGACTTTG
GGTTTTACGAACCTTTCTGGCTCGTCATTTTCGCCAATGTTTGCATTGCTGTCCAT
TTAGTAGGTGCCTATCAGGTATATGTTCAGCCTTTTTTCCAGTTTGTTGAGAGCAA
ATGTAACAAAAAGTGGCCTGAAAGCAATTTCATCAACAAAGAATACTCGTTGAAGA
TACCATTGCTCGGAAAATTTCGTGTCAACTTCTTCAGGCTGGTGTGGAGGACAAA
CTATGTGATTTTGACAACATTTATTGCAATGATATTCCCCTTCTTCAACTCCATCTT

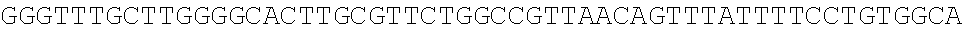

ATGCACATTGCTCAGACAAAGGTTAAGAAGTATTCGGGTAGATGGTTGGCGCTGA
ACCTCCTCGTGCTGGTTTGCTTGATTGTCTCCGCCCTAGCTGCTGTGGGATCCAT
TGTTGGCCTAATCAATAATGTCAAGAAATACAAGCCTTTCGAGAGTATAGACTAA
SEQ ID NO: 143: XP_009148321.1 AAP8 [Brassica rapa] (protein)
MSPSPPLTMKSLDTLHNPSAVESGNAAVKNVDDDGREKRTGTFLTASAHIITAVIGSG
VLSLAWALAQLGWVAGTMILVIFAIITYYTSTLLADCYRAPDPITGTRNYTYMGVVRAYL
GGKKVQLCGRAQYGNLVGVSIGYTITASISLVAIGRANCFHDKGHGAKCTASNYPYMV
AFGGLQILLSQIPNFHKLSFLSIIAAVMSFSYASIGIGLLAIAKVASGKVGKTTLLTGTVIGVD
VSASDKVWKAFQAVGDIAFSYSYTTILIEIQDTLRSSPPENKVMKKASLIGVSTTTVFYL
LCGCIGYAAFGNLLSPGDFLTDFGFYEPFWLVIFANVCIAVHLVGAYQVYVQPFFQFVE
SKCNKKWPESNFINKEYSLKIPLLGKFRVNFFRLVWRTNYVILTTFIAMIFPFFNSILGLL

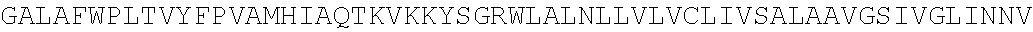

KKYKPFESID
SEQ ID NO: 144: XP_009148321 AAP8 [Brassica rapa] (genomic)
ATGTCTCCTTCTCCCCCTCTTACAATGAAATCCTTGGACACACTCCACAATCCCTC
GGCGGTTGAGTCCGGTAACGCCGCTGTGAAGAACGTCGACGATGATGGTCGAGA
GAAGAGAACGGGGACGTTTCTGACGGCGAGTGCGCACATTATCACGGCGGTGAT
AGGCTCAGGATGTGTTGTCTTTGGCTTGGGCATTAGCACAGCTTGGTTGGGTGGCT
GGAACCATGATTTTGGTGATTTTCGCCATCATCACTTACTACACGTCTACTTTGCT
CGCCGATTGCTACAGAGCGCCGGACCCCATCACCGGAACACGCAACTACACGTA
CATGGGCGTCGTTCGAGCTTACCTTGGTGGTAAAAAGGTGCAGCTATGTGGACGA
GCACAGTACGGAAACCTCGTTGGGGTCTCTATTGGTTACACCATCACTGCCTCCA
TAAGCTTAGTAGCGATTGGGAGAGCAAATTGTTTTCATGACAAGGGACcATGGTGC
GAAATGTACCGCATCGAATTATCCATACATGGTGGCATTTGGCGGCCTCCAGATT
CTTCTAAGTCAGATTCCTAATTTTCACAAGCTATCTTTCCTCTCAATCATTGCCGCG
GTTATGTCCTTCTCTTATGCATCTATTGGTATCGGTCTGGCCATCGCCAAAGTGGC
AAGTGGGAAGGTTGGTAAGACAACACTGACAGGTACGGTGATAGGAGTGGACGT
ATCTGCGTCTGATAAAGTGTGGAAAGCGTTTCAAGCGGTTGGGGATATTGCGTT
TTCGTACGCTTACACCACTATTCTCATTGAGATACAGGACACATTGAGATCAAGCC
CACCAGAGAACAAAGTGATGAAGAAAGCAAGTCTTATTGGAGTCTCAACCACAAC
TGTTTTCTACCTCTTATGTGGTTGCATTGGGTATGCTGCATTCGGAAACTTATCCC
CTGGTGACTTCCTTACCGACTTTGGGTTTTACGAACCTTTCTGGCTCGTCATTTTC
GCCAATGTTTGCATTGCTGTCCATTTAGTAGGTGCCTATCAGGTATATGTTCAGCC
TTTTTTCCAGTTTGTTGAGAGCAAATGTAACAAAAAGTGGCCTGAAAGCAATTTCAT
CAACAAAGAATACTCGTTGAAGATACCATTGCTCGGAAAATTTCGTGTCAACTTCT
TCAGGCTGGTGTGGAGGACAAACTATGTGATTTTGACAACATTTATTGCAATGATA

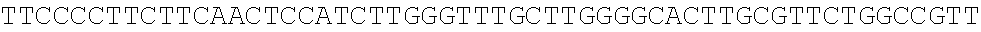

AACAGTTTATTTTCCTGTGGCAATGCACATTGCTCAGACAAAGGTTAAGAAGTATT
CGGGTAGATGGTTGGCGCTGAACCTCCTCGTGCTGGTTTGCTTGATTGTCTCCGC
CCTAGCTGCTGTGGGATCCATTGTTGGCCTAATCAATAATGTCAAGAAATACAAGC
CTTTCGAGAGTATAGACTAA
SEQ ID NO: 145: RID48756.1 hypothetical protein
BRARA_I015242 [Brassica rapa] (protein)
MLLSLSSLPRFFSSKMKSYATEYNPSAVETAGNNFDDDGREKRTGTLMTATAHIITAVI
GSGVLSLAWAIAQLGWVAGTVILVTFAVINYFTSTMLADCYRSPDTGIRNYNYMDVVR
AYLGGWKVKLCGLAQYGSLVGITIGYTITASISLVAIGKANCFHDKGHDAKCSVSNYPL
MAAFGITQIVLSQIHNFHKLSFLSIIATVMSFSYASIGIGLALAALASGKVGKTDLTGTVV
GVDVTASDKIWRSFQAAGDIAFSYAFSVVLVEIQDTLRSSPPENKVMKKASLAGVSTT
TGFYILCGCIGYAAFGNQAPGDFLTDFGFYEPYWLIDFANACIAVHLIAAYQVFAQPIFQ
FIEKKCNKAWPESNFIAKDYSINIPLLGKCRINFFRLVWRSTYVILTTVVAMIFPFFNAIL

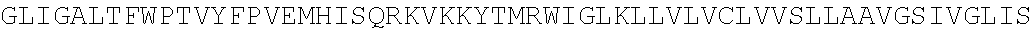

SVKAYKPFHNLD
SEQ ID NO: 146: RID48756.1 hypothetical protein BRARA_I05242
[Brassica rapa] (genomic)
ATGCTTTTATCACTTTCTTCTCTTCCTCGGTTTTTCTCGTCTAAAATGAAAAGCTAC
GCCACTGAGTATAATCCCTCGGCCGTGGAAACCGCCGGGAATAACTTCGACGAT
GATGGTCGGGAGAAGAGAACGGGGACGTTGATGACGGCGACCGCGCACATAATC
ACGGCGGTGATAGGTTCTGGAGTCTTGTCGTTGGCTTGGGCTATAGCACAACTTG
GTTGGGTGGCAGGAACGGTGATTTTGGTAACTTTTGCCGTTATAAATTACTTCACA
TCTACAATGCTTGCGGACTGCTATCGATCTCCGGACACAGGAATACGTAATTATAA
TTACATGGACGTTGTCAGAGCTTACCTTGGTGGTTGGAAAGTGAAGCTGTGTGGA
CTGGCACAGTACGGGAGTCTAGTAGGGATCACTATTGGCTACACCATCACTGCCT
CCATAAGCTTAGTAGCGATCGGGAAAGCAAATTGTTTTCATGACAAGGGACATGAT
GCAAAATGTTCCGTATCAAATTATCCACTCATGGCGGCGTTTGGTATCACCCAGAT
TGTTCTTAGTCAGATTCATAATTTTCACAAGCTCTCTTTTCTCTCCATTATCGCTAC
CGTTATGTCCTTCTCTTATGCATCCATCGGAATTGGCTTAGCCTTGGCTGCTCTGG
CAAGTGGGAAGGTTGGTAAGACGGATCTGACGGGCACGGTGGTTGGAGTAGACG
TAACTGCGTCTGACAAAATATGGAGGTCGTTTCAAGCAGCTGGAGACATTGCCTTT
TCGTACGCATTTTCCGTTGTTCTCGTTGAGATTCAGGATACACTGAGATCAAGCCC
ACCAGAGAACAAAGTCATGAAAAAAGCAAGCCTTGCTGGAGTTTCAACTACAACT
GGTTTCTACATCTTGTGTGGCTGCATCGGATATGCTGCTTTTGGAAACCAAGCCC
CTGGAGACTTCCTAACTGACTTTGGTTTTTATGAGCCTTACTGGCTCATTGATTTTG
CTAATGCTTGCATTGCTGTCCACCTAATCGCAGCCTATCAGGTGTTTGCACAACCA
ATATTCCAGTTTATTGAGAAGAAATGCAACAAAGCGTGGCCAGAAAGCAACTTCAT
CGCCAAAGATTATTCGATAAACATACCATTGCTAGGGAAATGTCGCATCAACTTCT
TCAGTTGGTCTGGAGGTCAACCTATGTGATTTTGACAACAGTTGTAGCGATGAT

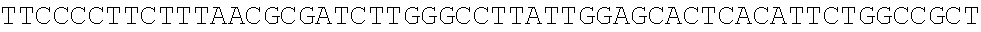

AACAGTTTACTTCCCAGTGGAGATGCACATCTCGCAGAGAAAGGTTAAGAAGTATA
CTATGAGATGGATAGGGTTGAAACTCCTTGTATTGGTTTGTTTGGTTGTTTCGCTC
CTAGCTGCAGTAGGATCCATTGTCGGCTTGATAAGTAGTGTAAAGGCATACAAGC
CTTTCCACAATTTAGATTAG
SEQ ID NO: 147: XP_009118279.1 PREDICTED: amino acid permease 8-like
[Brassica rapa] (protein)
MLLSLSSLPRFFSSKMKSYATEYNPSAVETAGNNFDDDGREKRTGTLMTATAHIITAVI
GSGVLSLAWAIAQLGWVAGTVILVTFAVINYFTSTMLADCYRSPDTGIRNYNYMDVVR
AYLGGWKVKLCGLAQYGSLVGITIGYTITASISLVAIGKANCFHDKGHDAKCSVSNYPL
MAAFGITQIVLSQIHNFHKLSFLSIIATVMSFSYASIGIGLALAALASGKVGKTDLTGTVV
GVDVTASDKIWRSFQAAGDIAFSYAFSVVLVEIQDTLRSSPPENKVMKKASLAGVSTT
TGFYILCGCIGYAAFGNQAPGDFLTDFGFYEPYWLIDFANACIAVHLIAAYQFAQPIFQ
FIEKKCNKAWPESNFIAKDYSINIPLLGKCRINFFRLVWRSTYVILTTVVAMIFPFFNAIL

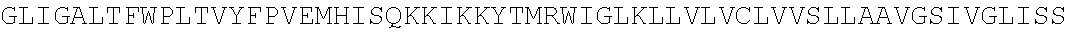

VKAYKPFHNLD
SEQ ID NO: 148: XP_009118279.1 PREDICTED: amino acid permease 8-like
[Brassica rapa] (genomic)
ATGCTTTTATCACTTTCTTCTCTTCCTCGGTTTTTCTCGTCTAAAATGAAAAGCTAC
GCCACTGAGTATAATCCCTCGGCCGTGGAAACCGCCGGGAATAACTTCGACGAT
GATGGTCGGGAGAAGAGAACGGGGACGTTGATGACGGCGACCGCGCACATAATC
ACGGCGGTGATAGGTTCTGGAGTCTTGTCGTTGGCTTGGGCTATAGCACAACTTG
GTTGGGTGGCAGGAACGGTGATTTTGGTAACTTTTGCCGTTATAAATTACTTCACA
TCTACAATGCTTGCGGACTGCTATCGATCTCCGGACACAGGAATACGTAATTATAA
TTACATGGACGTTGTCAGAGCTTACCTTGGTGGTTGGAAAGTGAAGCTGTGTGGA
CTGGCACAGTACGGGAGTCTAGTAGGGATCACTATTGGCTACACCATCACTGCCT
CCATAAGCTTAGTAGCGATCGGGAAAGCAAATTGTTTTCATGACAAGGGACATGAT
GCAAAATGTTCCGTATCAAATTATCCACTCATGGCGGCGTTTGGTATCACCCAGAT
TGTTCTTAGTCAGATTCATAATTTTCACAAGCTCTCTTTTCTCTCCATTATCGCTAC
CGTTATGTCCTTCTCTTATGCATCCATCGGAATTGGCTTAGCCTTGGCTGCTCTGG
CAAGTGGGAAGGTTGGTAAGACGGATCTGACGGGCACGGTGGTTGGAGTAGACG
TAACTGCGTCTGACAAAATATGGAGGTCGTTTCAAGCAGCTGGAGACATTGCCTTT
TCGTACGCATTTTCCGTTGTTCTCGTTGAGATTCAGGATACACTGAGATCAAGCCC
ACCAGAGAACAAAGTCATGAAAAAGCAAGCCTTGCTGGAGTTTCAACTACAACT
GGTTTCTACATCTTGTGTGGCTGCATCGGATATGCTGCTTTTGGAAACCAAGCCC
CTGGAGACTTCCTAACTGACTTTGGTTTTTATGAGCCTTACTGGCTCATTGATTTTG
CTAATGCTTGCATTGCTGTCCACCTAATCGCAGCCTATCAGGTGTTTGCACAACCA
ATATTCCAGTTTATTGAGAAGAAATGCAACAAAGCGTGGCCAGAAAGCAACTTCAT
CGCCAAAGATTATTCGATAAACATACCATTGCTAGGGAAATGTCGCATCAACTTCT
TCAGATTGGTCTGGAGGTCAACCTATGTGATTTTGACAACAGTTGTAGCGATGATA

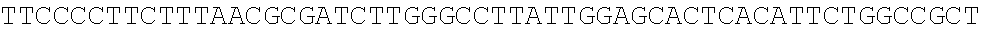

AACAGTTTACTTCCCAGTGGAGATGCACATCTCGCAGAAAAAGATTAAGAAGTATA
CTATGAGATGGATAGGGTTGAAACTCCTTGTATTGGTTTGTTTGGTTGTTTCGCTC
CTAGCTGCAGTAGGATCCATTGTCGGCTTGATAAGTAGTGTAAAGGCATACAAGC
CTTTCCACAATTTAGATTAG
SEQ ID NO: 149: RID48754.1 hypothetical protein BRARA_I05240
[Brassica rapa] (protein)
MLLHISFLSSSVSSPLKMKSFDTSSVVESGAGAGNNVDDDCREKRTGTLITASAHIITTVI
GSGVLSLAWAIAQLGWVVGTVILVAFAVIVNYTSRMLADSYRSPEGTRNYTYMDVVR
VYLGGRKVQLCGLAQFGSLVGVTIGYTITASISLVAIGKANCFHDKGHGAKCSVSNYPL
MAAFGIVQIFLSQIPNFHKLSFLSIIATVMSFSYASIGFGLALAALASGKVGKTGLTGTVV
GVDVTASDKLWKSFQAAGNIAFSYAYSVVLVEIQDTLRSSPPENKVMKKASLAAVSTT
TAFYILCGCIGYATFGNQAPGDFLTDFGFYEPYWLIDFANACIAVHLIGAYQVFAQPIFQ
FVEKKCNQAWPESNFITKEHSMNIPLLGKCRINFFRLVWRTTYVIFSTVVAMIFPFFNAI

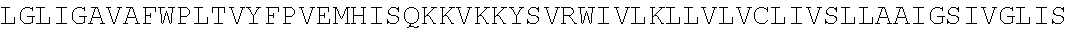

SVKAYKPFHNLD
SEQ ID NO: 150: RID48754.1 hypothetical protein BRARA_I05240
[Brassica rapa] (genomic)
ATGCTTTTGCATATCTCTTTTCTCTCTTCTTCAGTTTCTCCTCTCAAAATGAAAAGCT
TCGACACGAGCTCAGTGGTTGAATCCGGTGCTGGCGCCGGGAATAACGTCGACG
ATGATTGTCGGGAGAAGAGAACGGGGACCTTGATAACGGCGAGTGCCCACATAA
TCACGACAGTGATAGGTTCTGGAGTCTTGTCGTTGGCTTGGGCTATAGCACAACT
TGGTTGGGTGGTQGGAACAGTGATTTTGGTAGCCTTTGCCGTCATAGTTAATTACA
CATCCAGAATGCTCGCCGACAGTTATCGATCCCCGGAGGGAACACGCAACTATAC
TTACATGGACGTCGTCCGAGTCTACCTTGGTGGTAGGAAAGTGCAGCTGTGTGGA
CTAGCACAGTTCGGGAGTCTCGTAGGGGTTACTATTGGTTACACCATCACTGCCT
CCATAAGCTTAGTGGCGATTGGGAAAGCAAATTGTTTTCATGACAAGGGACATGG
TGCGAAATGTTCCGTATCAAATTATCCACTCATGGCGGCGTTTGGAATCGTCCAGA
TTTTTCTTAGTCAGATTCCTAATTTTCACAAGCTCTCTTTTCTCTCCATTATCGCCAC
CGTTATGTCCTTCTCTTATGCATCTATCGGTTTTGGCTTAGCCTTGGCCGCTCTGG
CAAGTGGGAAGGTTGGTAAGACGGGACTGACAGGCACGGTGGTTGGAGTGGATG
TAACTGCGTCTGACAAATTATGGAAGTCATTTCAAGCGGCTGGAAACATTGCCTTT
TCATACGCTTATTCCGTTGTTCTCGTTGAGATTCAGGACACACTGAGATCAAGCCC
ACCAGAGAACAAAGTCATGAAAAAGCAAGCCTTGCTGCAGTCTCAACTACAACT
GCTTTCTACATCTTATGTGGCTGCATCGGATATGCTACATTTGGAAACCAAGCCCC
CGGAGACTTCCTTACTGACTTTGGTTTTTATGAACCTTACTGGCTCATCGATTTTG
CTAATGCTTGCATCGCTGTCCACCTTATCGGAGCTTATCAGGTGTTTGCACAACCA
ATATTCCAGTTTGTTGAGAAGAAATGCAATCAGGCGTGGCCAGAAAGCAACTTCAT
CACCAAAGAACATTCGATGAACATACCGTTGCTTGGAAAATGTCGCATTAACTTCT
TCAGACTGGTGTGGAGGACAACCTATGTGATTTTCTCAACAGTTGTAGCAATGATA

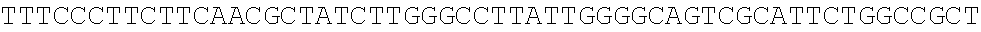

AACAGTTTACTTCCCGGTGGAGATGCACATCTCGCAGAAAAAGGTTAAGAAGTATT
CTGTGAGATGGATAGTATTGAAACTCCTTGTTTTGGTTTGTTTAATTGTTTCGCTCC
TAGCTGCCATAGGATCCATCGTTGGCTTGATAAGTAGTGTCAAGGCATACAAGCC
TTTCCACAATTTAGATTAG
SEQ ID NO: 151: XP_009118276.1 AAP8-like isoform X2 [
Brassica rapa] (protein)
MKSFDTSSVVESGAGAGNNVDDDCCREKRTGTLITASAHIITTVIGSGVLSLAWAIAQLG
WVVGTVILVAFAVIVNYTSRMLADSYRSPEGTRNYTYMDVVRVYLGGRKVQLCGLAQ
FGSLVGVTIGYTITASISLVAIGKANCFHDKGHGAKCSVSNYPLMAAFGIVQIFLSQIPN
FHKLSFLSIIATVMSFSYASIGFGLALAALASGKVGKTGLTGTVVGVDVTASDKLWKSF
QAAGNIAFSYAYSVVLVEIQDTLRSSPPENKVMKKASLAAVSTTTAFYILCGCIGYATF
GNQAPGDFLTDFGFYEPYWLIDFANACIAVHLIGAYQVFAQPIFQFVEKKCNQAWP

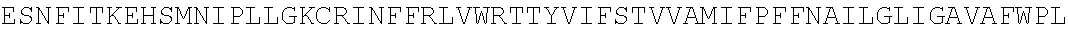

TVYFPVEMHISQKKVKKYSVRWIVLKLLVLVCLIVSLLAAIGSIVGLISSVKAYKPFHNLD
SEQ ID NO: 152: XP_009118276.1 AAP8-like isoform X2 [
Brassica rapa] (genomic)
ATGAAAAGCTTCGACACGAGCTCAGTGGTTGAATCCGGTGCTGGCGCCGGGAAT
AACGTCGACGATGATTGTCGGGAGAAGAGAACGGGGACCTTGATAACGGCGAGT
GCCCACATAATCACGACAGTGATAGGTTCTGGAGTCTTGTCGTTGGCTTGGGCTA
TAGCACAACTTGGTTGGGTGGTAGGAACAGTGATTTTGGTAGCCTTTGCCGTCAT
AGTTAATTACACATCCAGAATGCTCGCCGACAGTTATCGATCCCCGGAGGGAACA
CGCAACTATACTTACATGGACGTCGTCCGAGTCTACCTTGGTGGTAGGAAAGTGC
AGCTGTGTGGACTAGCACAGTTCGGGAGTCTCGTAGGGGTTACTATTGGTTACAC
CATCACTGCCTCCATAAGCTTAGTGGCGATTGGGAAAGCAAATTGTTTTCATGACA
AGGGACATGGTGCGAAATGTTCCGTATCAAATTATCCACTCATGGCGGCGTTTGG
AATCGTCCAGATTTTTCTTAGTCAGATTCCTAATTTTCACAAGCTCTCTTTTCTCTC
CATTATCGCCACCGTTATGTCCTTCTCTTATGCATCTATCGGTTTTGGCTTAGCCTT
GGCCGCTCTGGCAAGTGGGAAGGTTGGTAAGACGGGACTGACAGGCACGGTGG
TTGGAGTGGATGTAACTGCGTCTGACAAATTATGGAAGTCATTTCAAGCGGCTGG
AAACATTGCCTTTTCATACGCTTATTCCGTTGTTCTCGTTGAGATTCAGGACACACT
GAGATCAAGCCCACCAGAGAACAAAGTCATGAAAAAAGCAAGCCTTGCTGCAGTC
TCAACTACAACTGCTTTCTACATCTTATGTGGCTGCATCGGATATGCTACATTTGG
AAACCAAGCCCCCCGGAGACTTCCTTACTGACTTTGGTTTTTATGAACCTTACTGGC
TCATCGATTTTGCTAATGCTTGCATCGCTGTCCACCTTATCGGAGCTTATCAGGTG
TTTGCACAACCAATATTCCAGTTTGTTGAGAAGAAATGCAATCAGGCGTGGCCAGA
AAGCAACTTCATCACCAAAGAACATTCGATGAACATACCGTTGCTTGGAAAATGTC
GCATTAACTTCTTCAGACTGGTGTGGAGGACAACCTATGTGATTTTCTCAACAGTT

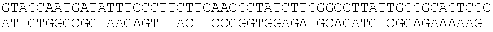

GTTAAGAAGTATTCTGTGAGATGGATAGTATTGAAACTCCTTGTTTTGGTTTGTTTA
ATTGTTTCGCTCCTAGCTGCCATAGGATCCATCGTTGGCTTGATAAGTAGTGTCAA
GGCATACAAGCCTTTCCACAATTTAGATTAG
SEQ ID NO: 153: RID57272.1 hypotetical protein BRARA_F00659
[Brassica rapa] (protein)
MSPSPPLTMKSLDTLHNPSAVESGNAAVKNVDDDGREKRTGTFLTASAHIITAVIGSG
VLSLAWALAQLGWVAGTMILVIFAIITYYTSTLLADCYRAPDPITGTRNYTYMGVVRAYL
GGKKVQLCGLAQYGNLVGVSIGYTITASISLVAIGRANCFHDKGHGAKCTASNYPYMV
AFGGLQILLSQIPNFHKLSFLSIIAAVMSFSYASIGIGLAIAKVASGKVGKTTLTGTVIGVD
VSASDKVWKAFQAVGDIAFSYAYTTILIEIQDTLRSSPPENKVMKKASLIGVSTTTVFYL
LCGCIGYAAFGNLSPGDFLTDFGFYEPFWLVIFANVCIAVHLVGAYQVYVQPFFQFVE
SKCNKKWPESNFINKEYSLKIPLLGKFRVNFFRLVWRTNYVILTTFIAMIFPFFNSILGLL

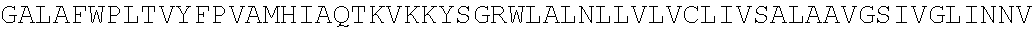

KKYKPFESID
SEQ ID NO: 154: RID57272.1 hypothetical protein BRARA_F00659
[Brassica rapa] (genomic)
ATGAAAAGCTTTGACGCGGTGCATAATCCCTCTGCGGTGGAATCCGCTGACGCCA
ACGTCGACGATGATGGTCGGGAGAAGAGAACGGGGACGTTGATGACGGCGAGT
GCGCACATAATCACGGCTGTGATAGGTTCCGGAGTGTTGTCGTTGGCTTGGGCTA
TAGCACAACTTGGTTGGGTGGCAGGAACATTGATTCTTGTAACTTTTGCCGTCGTC
AATTACTACACATCCACTATGCTCGCCGATTGTTATAGATCGGACGCAGGAGCTC
GCAACTATACGTACATGGACGTCGTTCGATCTTACCTTGGTGGTAGGAAAGTGCA
GTTATGTGGACTGGCACAATACGGGTGTCTCGTAGGGGTCACTATTGGTTACACC
ATCACTGCGTCTATAAGTTTAGTAGCGATTTGGAAAGCAACTTGTTTTCATAAAAAA
GGACATGGTGCAAAATGCTCCATCCCAAATTATCCATTCATGGTGGCCTTCGGGG
TCGTGGAGATTCTTCTTAGTCAGCTTCCTAATTTTCACAAGCTCTCTTTTCTCTCCA
TTATCGCCGCCATTATGTCATTCTCTTATGCGTCTATCGGAATTGGTTTAGCCATTT
CCGTTGTGGCAAGTGGAAAGGTTGGTAAGACGAGTGTGACGGGCACGGTGGTTG
GAGTGGACGTGACCGCATCTGACAAAATATGGAAGGCGTTTCAAGCAACTGGAGA
CATTGCATTTTCATACTCTTTTTCCACTATTCTCGTTGAGATTCAGGATACATTGAG
ATCAAACCCACCAGAAAACAAAGTCATGAAAAAGCAACACTTGCCGGAGTCTCA
ACTACAACTGTTTTCTACATCTTATGTGGCTGCATGGGATATGCTGCATTTGGAAA
CCGAGCCCCCGGAGACTTCCTTACTGACTTTGGTTTTTATGAACCTTACTGGCTCA
TCAATTTTGCCAATGCTTGCATCGTCCTCCACCTAATTGCAGCCTATCAGGTGTTT
GCACAACCAATTTTCCAACTTGTTGAGAACAAATGCAACAAAGCATGGCCAGAAAA
CAATTTCATCCACAAAGAACATTCGATAAACATACTATTCCTCGGAAAATGGCGCA
TCAACTTCTTCAGACTGGTGTGGAGGACAGCATATGTGATTTTGACAACATTTGTT

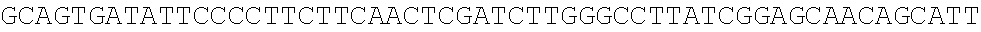

CTGGCCGCTAACAGTTTACTTCCCAGTGGAGATGCACATCTCGCAGAGAAAGGTT
AAGAAGTATTCTATGAAATGGAATGCGTTGAAACTCCTTATATCGGTTTGTTTGATT
GTTTCGCTCCTAGCTGCAATAGGATCCATTGTCGGCTTGATAAATAGTGTCAAGGC
ATACAAGCCTTTCCATAGTTAA
BRASSICA OLERACEA
SEQ ID NO: 155: VDD42023.1 unnamed protein product
[Brassica oleracea] (protein)
MSPSPPPTMKSLDTLHNPSAVESGNAAVKNVDDDGREKRTGTFLTASAHIITAVIGSG
VLSLAWALAQLGWVAGTMILVIFAIITYYTSTLLADCYRAPDPITGTRNYTYMGVVRAYL
GGKKVQLCGLAQYGNLVGVSIGYTITASISLVAIGKANCFHGKGHGAKCTASNYPYMV
AFGGLQILLSQIPNFHKLSFLSIIAAVMSFSYASIGIGLAIAKVASGKVGKTTLTGTVIGVD
VSASDKVWKAFQAVGDIAFSYAYTTILIEIQDTLRSSPPENKVMKKASLIGVSTTTVFYL
LCGCIGYAAFGNIAPGDFLTDFGFYEPFWLVIFANVCIAVHLVGAYQVYVQPFFQFVES
KCNKKWPESNFINKEYSLKIPLLGKFRVNHFRLVWRTNYVILTTFIAMIFPFFNSILGLLG

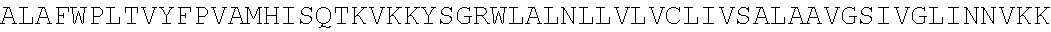

YKPFESID
SEQ ID NO: 156: VDD42023.1 unnamed protein product
[Brassica oleracea] (genomic)
ATGTCTCCCTCTCCCCCTCCTACAATGAAATCCTTGGACACACTCCACAATCCCTC
GGCGGTTGAGTCCGGTAACGCCGCTGTGAAGAACGTCGACGATGATGGTCGAGA
GAAGAGAACGGGGACGTTTCTGACGGCGAGTGCGCACATTATCACGGCGGTGAT
AGGCTCAGGAGTGTTGTCTTTGGCTTGGGCATTAGCACAGCTTGGTTGGGTGGCT
GGAACCATGATTTTGGTGATTTTCGCCATCATTACTTACTACACCTCTACTTTGCTC
GCCGATTGCTACAGAGCGCCGGACCCCATCACCGGAACACGCAACTACACGTAC
ATGGGCGTCGTTCGAGCTTACCTTGGTGGTAAAAAGGTGCAGCTATGTGGACTAG
CACAGTACGGCAACCTCGTTGGGGTCTCTATTGGTTACACCATCACTGCCTCCAT
AAGCTTAGTAGCGATTGGGAAAGCAAATTGTTTTCATGGTAAGGGACATGGTGCG
AAATGTACCGCATCGAATTATCCATACATGGTGGCATTTGGCGGCCTCCAGATTCT
TCTAAGTCAGATTCCTAATTTTCACAAGCTATCTTTCCTCTCAATCATTGCCGCGGT
TATGTCCTTCTCTTATGCATCTATTGGTATCGGTCTGGCCATCGCCAAAGTGGCAA
GTGGGAAGGTTGGTAAGACAACGCTGACAGGTACGGTGATAGGAGTGGACGTAT
CTGCGTCTGATAAAGTATGGAAAGCGTTTCAAGCGGTTGGGGATATTGCGTTTTC
GTACGCTTACACCACTATTCTCATTGAGATCCAGGACACATTGAGATCAAGCCCAC
CAGAGAACAAAGTGATGAAGAAAGCAAGTCTTATTGGAGTCTCAACCACAACTGTT
TTCTACCTCTTATGTGGTTGCATTGGGTATGCTGCATTCGGAAACATAGCCCCTGG
TGACTTCCTTACCGACTTTGGGTTTTACGAACCTTTCTGGCTCGTCATTTTCGCCA
ATGTTTGCATTGCTGTCCATTTAGTAGGTGCCTATCAGGTATATGTTCAGCCCTTTT
TCCAATTTGTTGAGAGCAAATGCAACAAAAAGTGGCCTGAAAGCAATTTCATCAAC
AAAGAGTACTCGTTGAAGATACCATTGCTCGGAAAATTTCGTGTCAACCACTTCAG
GCTGGTGTGGAGGACAAACTATGTGATTTTGACAACATTTATTGCAATGATATTCC

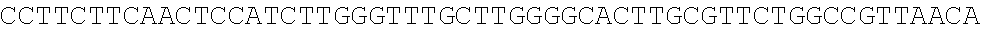

GTTTATTTTCCTGTGGCAATGCACATTTCTCAGACAAAGGTTAAGAAGTATTCGGG
TAGATGGTTGGCGCTGAACCTCCTCGTGTTGGTTTGCTTGATTGTCTCCGCCTTA
GCTGCAGTGGGATCCATTGTTGGTCTAATCAATAATGTCAAGAAATACAAGCCTTT
CGAGAGTATAGACTAA
SEQ ID NO: 157: XP_013586575.1 PREDICTED: amino acid permease
8 [Brassica oleracea var. oleracea] (protein)
MSPSPPPTMKSLDTLHNPSAVESGNAAVKNVDDDGREKRTGTFLTASAHIITAVIGSG
VLSLAWALAQLGWVAGTMILVIFAIITYYTSTLLADCYRAPDPITGTRNYTYMGVVRAYL
GGKKVQLCGLAQYGNLVGVSIGYTITASISLVAIGKANCFHGKGHGAKCTASNYPYMG
AFGGLQILLSQIPNFHKLSFLSIIAAVMSFSYASIGIGLAIAKVASGKVGKTTLTGTVIGVD
VSASDKVWKAFQAVGDIAFSYAYTTILIEIQDTLRSSPPENKVMKKASLIGVSTTTVFYL
LCGCIGYAAFGNIAPGDFLTDFGFYEPFWLVIFANVCIAVHLVGAYQVYVQPFFQFVES
KCNKKWPESNFINKEYSLKIPLLGKFRVNHFRLVWRTNYVILTTFIAMIFPFFNSILGLLG

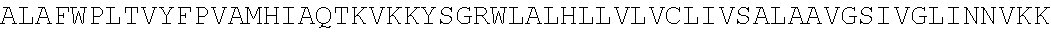

YKPFESID
SEQ ID NO: 158: XP_013586575.1 PREDICTED: amino acid permease
8 [Brassica oleracea var. oleracea] (genomic)
ATGTCTCCCTCTCCCCCTCCTACAATGAAATCCTTGGACACACTCCACAATCCCTC
GGCGGTTGAGTCCGGTAACGCCGCTGTGAAGAACGTCGACGATGATGGTCGAGA
GAAGAGAACGGGGACGTTTCTGACGGCGAGTGCGCACATTATCACGGCGGTGAT
AGGCTCAGGAGTGTTGTCTTTGGCTTGGGCATTAGCACAGCTTGGTTGGGTGGCT
GGAACCATGATTTTGGTGATTTTCGCCATCATTACTTACTACACCTCTACTTTGCTC
GCCGATTGCTACAGAGCGCCGGACCCCATCACCGGAACACGCAACTACACGTAC
ATGGGCGTCGTTCGAGCTTACCTTGGTGGTAAAAAGGTGCAGCTATGTGGACTAG
CACAGTACGGCAACCTCGTTGGGGTCTCTATTGGTTACACCATCACTGCCTCCAT
AAGCTTAGTAGCGATTGGGAAAGCAAATTGTTTTCATGGTAAGGGACATGGTGCG
AAATGTACCGCATCGAATTATCCATACATGGGGGCATTTGGCGGCCTCCAGATTC
TTCTAAGTCAGATTCCTAATTTTCACAAGCTATCTTTCCTCTCAATCATTGCCGCGG
TTATGTCCTTCTCTTATGCATCTATTGGTATCGGTCTGGCCATCGCCAAAGTGGCA
AGTGGGAAGGTTGGTAAGACAACGCTGACAGGTACGGTGATAGGAGTGGACGTA
TCTGCGTCTGATAAAGTATGGAAAGCGTTTCAAGCGGTTGGGGATATTGCGTTTTC
GTACGCTTACACCACTATTCTCATTGAGATCCAGGACACATTGAGATCAAGCCCAC
CAGAGAACAAAGTGATGAAGAAAGCAAGTCTTATTGGAGTCTCAACCACAACTGTT
TTCTACCTCTTATGTGGTTGCATTGGGTATGCTGCATTCGGAAACATAGCCCCTGG
TGACTTCCTTACCGACTTTGGGTTTTACGAACCTTTCTGGCTCGTCATTTTCGCCA
ATGTTTGCATTGCTGTCCATTTAGTAGGTGCCTATCAGGTATATGTTCAGCCCTTTT
TCCAATTTGTTGAGAGCAAATGCAACAAAAAGTGGCCTGAAAGCAATTTCATCAAC
AAAGAGTACTCGTTGAAGATACCATTGCTCGGAAAATTTCGTGTCAACCACTTCAG
GCTGGTGTGGAGGACAAACTATGTGATTTTGACAACATTTATTGCAATGATATTCC

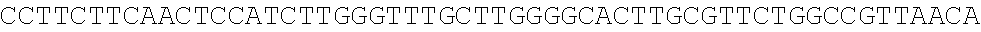

GTTTATTTTCCTGTGGCAATGCACATTGCTCAGACAAAGGTTAAGAAGTATTCGGG
TAGATGGTTGGCGCTGCACCTCCTCGTGTTGGTTTGCTTGATTGTCTCCGCCTTA
GCTGCAGTGGGATCCATTGTTGGCCTAATCAATAATGTCAAGAAATACAAGCCTTT
CGAGAGTATAGACTAA
SEQ ID NO: 159: XP_013599620.1 PREDICTED: amino acid permease 8-like
[Brassica oleracea var. oleracea] (protein)
MKSFHTEYNPSAVEAAGNNFDDDGREKRTGTVMTASAHIITAVIGSGVLSLAWAIAQL
GWVAGTVILVTFAVINYFTSTMLADCYRSPDTGIRNYNYMDVVRAYLGGWKVKLCGL
AQYGSLVGITIGYTITASISLVAIGKANCFHEKGHGAKCSVSNYPLMAAFGIIQIVLSQIH
NFHKLSFLSIIATVMSFSYASIGIGLALAALASGKVGKTDLTGTVVDVDVTASDKIWRSF
QAAGDIAFSYAFSVVLVEIQDTLRSSPPENKVMKKASLAGVSTTTGFYILCGCIGYAAF
GNQAPGDFLTDFGFYEPYWLIDFANACIAVHLIAAYQVFAQPIFQFIEKKCNKAWPESN

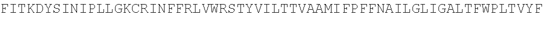

PVEMHISQKKVKKYTMRWIGLKLLVLVCLVVSLLAAVGLISSVKAYPKFHNLD
SEQ ID NO: 160: XP_013599620.1 PREDICTED: amino acid permease 8-like
[Brassica oleracea var. oleracea] (genomic)
ATGAAAAGCTTCCACACTGAGTATAATCCCTCGGCCGTGGAAGCCGCCGGGAATA
ACTTCGACGACGATGGTCGGGAGAAGAGAACGGGGACGGTGATGACGGCAAGT
GCTCACATTATCACTGCTGTGATAGGTTCCGGAGTCTTGTCCTTGGCTTGGGCTAT
AGCACAACTTGGTTGGGTGGCAGGAACAGTGATTTTGGTAACTTTTGCCGTTATAA
ATTACTTCACATCTACAATGCTTGCCGACTGTTATCGATCTCCGGACACAGGAATA
CGTAATTATAATTACATGGACGTTGTCAGAGCTTACCTTGGTGGTTGGAAAGTGAA
GCTATGTGGTCTGGCACAGTACGGGAGTCTAGTAGGGATCACTATTGGTTACACC
ATCACTGCCTCCATAAGCTTAGTAGCGATAGGGAAAGCAAATTGTTTTCATGAAAA
GGGACATGGTGCAAAATGTTCCGTATCGAATTATCCACTCATGGCGGCGTTTGGT
ATCATCCAGATTGTTCTTAGTCAGATTCATAATTTTCACAAGCTCTCTTTTCTCTCC
ATTATCGCCACCGTTATGTCCTTCTCTTATGCATCCATCGGAATTGGCTTGGCCTT
GGCCGCTCTGGCAAGTGGGAAGGTTGGTAAGACGGATCTGACGGGCACGGTGG
TTGGAGTAGACGTAACTGCGTCTGACAAAATATGGAGGTCGTTTCAAGCAGCTGG
AGACATTGCCTTTTCGTACGCATTTTCCGTTGTTCTCGTTGAGATTCAGGATACAC
TGAGATCAAGCCCACCAGAGAACAAAGTCATGAAAAAAGCAAGCCTTGCTGGAGT
TTCAACTACAACTGGTTTCTACATCTTATGTGGCTGCATCGGATATGCTGCTTTTG
GAAACCAAGCCCCTGGAGACTTCCTAACTGACTTTGGTTTTTATGAGCCTTACTGG
CTCATTGATTTTGCTAATGCTTGCATTGCTGTCCACCTAATCGCAGCCTATCAGGT
GTTTGCACAACCAATATTCCAGTTTATTGAGAAGAAATGCAACAAAGCGTGGCCAG
AAAGCAACTTTATCACCAAAGATTATTCGATAAACATACCATTGCTAGGGAAATGT
CGCATCAACTTCTTCAGATTGGTCTGGAGGTCAACCTATGTGATTTTGACAACAGT

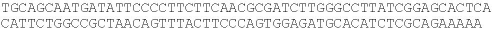

GGTTAAGAAGTATACTATGAGATGGATAGGGTTGAAACTCCTTGTATTGGTTTGTT
TGGTTGTTTCGCTCCTAGCTGCAGTAGGATCCATTGTCGGCCTCATAAGTAGTGTA
AAGGCATACAAGCCTTTCCACAATTTAGATTAG
SEQ ID NO: 161: XP_013584691.1 PREDICTED: amino acid permease 8-like
[Brassica oleracea var. oleracea] (protein)
MKSFDAVHNPSAVESADANVDDDGREKRTGTLMTASAHIITAVIGSGVLSLAWAIAQL
GWVAGTLILVTFAIVNYYSTMLADCYRSDAGARNYTYMDVVRSYLGGRKVQLCGLA
QYGCLVGVTIGYTITASISLVAIWKATCFHKKGHGAKCSIPNYPFMAAFGVVEIFLSQLP
NFHKLSFLSIIAAVMSFSYASIGIGLAIAVVASGKVGKTGVTGTVVGVDVTASDKIWKAF
QATGDIAFSYSFSTILVEIQDTLRSSPPENKVMKKATLAGVSTTTVFYILCGCMGYAAF
GNRAPGDFLTDFGFYEPYWLINFANACIVLHLIAAYQVFAQPIFQLVENKCNKAWPEN

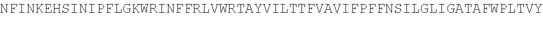

FPVEMHISQRKVKKFSMKWNALKLLVLVCLIVSLLAAIGSIVGLINSVKAYKPFHS
SEQ ID NO: 162 XP_013584691.1 PREDICTED: amino acid permease 8-like
[Brassica oleracea var. oleracea] (genomic)
ATGAAAAGCTTTGACGCGGTGCATAATCCCTCTGCGGTGGAATCCGCTGACGCCA
ACGTCGACGATGATGGTCGGGAGAAGAGAACGGGGACGTTGATGACGGCGAGT
GCGCACATAATCACGGCGGTGATAGGTTCCGGAGTGTTGTCGTTGGCCTGGGCT
ATAGCACAGCTTGGTTGGGTGGCAGGAACACTGATTCTTGTAACTTTTGCCATCGT
CAATTAACTACACATCCACTATGCTCGCCGACTGTTATAGATCGGACGCAGGAGCT
CGCAACTATACGTACATGGACGTCGTCCGATCTTACCTTGGTGGTAGGAAAGTGC
AGTTATGTGGACTGGCACAATACGGGTGTCTCGTAGGGGTCACTATTGGTTACAC
CATCACTGCCTCTATAAGTTTAGTAGCGATTTGGAAAGCAACTTGTTTTCATAAAAA
AGGACATGGTGCGAAATGTTCCATCCCAAATTATCCATTCATGGCGGCCTTCGGG
GTCGTGGAGATTTTTCTTAGTCAGCTTCCTAATTTTCACAAGCTCTCTTTTCTCTCC
ATTATCGCCGCCGTTATGTCATTCTCTTATGCGTCTATCGGAATTGGTTTAGCCAT
TGCCGTTGTGGCAAGTGGAAAGGTTGGTAAGACGGGTGTGACGGGCACGGTGGT
TGGAGTGGACGTGACCGCATCTGACAAAATATGGAAGGCGTTTCAAGCAACTGGA
GACATTGCATTTTCATACTCTTTTTCCACTATTCTCGTTGAGATTCAGGATACATTG
AGATCAAGCCCACCAGAAAACAAAGTCATGAAAAAAGCAACACTCGCCGGAGTCT
CAACTACAACTGTTTTCTACATCTTATGTGGCTGCATGGGATATGCTGCATTTGGA
AACCGAGCCCCCGGAGACTTCCTTACTGACTTTGGTTTTTATGAACCTTACTGGCT
CATCAACTTTGCCAATGCTTGCATCGTCCTCCACCTAATCGCAGCCTATCAGGTGT
TTGCACAACCAATTTTCCAACTTGTTGAGAACAAATGCAACAAAGCATGGCCAGAA
AACAATTTCATCAACAAAGAACATTCGATAAACATACCATTCCTCGGAAAATGGCG
CATCAACTTCTTCAGACTGGTGTGGAGGACAGCATATGTGATTTTGACAACATTTG

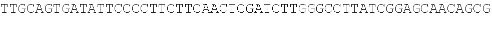

TTCTGGCCGCTAACAGTTTACTTCCCAGTGGAGATGCACATCTCGCAGAGAAAGG
TTAAGAAGTTTTCTATGAAATGGAATGCGTTGAAACTCCTTGTATTGGTTTGTTTGA
TTGTTTCGCTCCTAGCTGCAATAGGATCCATCGTCGGCTTGATAAATAGTGTCAAG
GCATACAAGCCTTTCCATAGTTAA
SEQ ID NO: 163: XP_013601938.1 AAP8-like [Brassica
oleracea var. oleracea] (protein)
MLLHISFISSSVSPLKMKSFDTSSVVESGAGAGNNVDDDCREKRTGTLITASAHIITTVI
GSGVLSLAQAIAQLGWVVGTVILVAFAVIVNYTSRMLADSYRSPEGTRNYTYMDVVR
VYLGGRKVQLCGLAQFGSLVFVTIGYTITASISLVAIGKANCFHDKGHGADCSVSNYPL
MAAFGIVQIFLSQIPNFHKLSFLSIIATVMSFSYASIGFGLALAALASGKVGKTGLTGTVV
RVDVTASDKLWKSFQAAGNIAFSYAYSVVLVEIQDTLRSSPPENKVMKKASLAAVSTT
TAFYILCGCIGYATFGNQAPGDFLTDFGFYEPYWLIDFANACIAVHLIGAYQVFAQPIFQ
FVEKKCNQAWPESNFITKEPSMNVPLLGKCRINFFRLVWRTTYVIFSTVVAMIFPFFNA

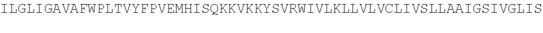

SVKAYKPFHNLD
SEQ ID NO: 164: XP_013601938.1 AAP8-like [Brassica
oleracea var. oleracea] (genomic)
ATGCTTTTGCATATCTCTTTTATCTCTTCTTCAGTTTCTCCTCTCAAAATGAAAAGCT
TCGACACGAGCTCAGTGGTTGAATCCGGTGCTGGCGCCGGGAATAACGTCGACG
ATGATTGTCGGGAGAAGAGAACGGGGACGTTGATAACGGCGAGTGCCCACATAA
TCACGACAGTGATAGGTTCTGGAGTCTTGTCGTTGGCTTGGGCTATAGCACAACT
TGGTTGGGTGGTAGGAACAGTGATTTTGGTAGCCTTTGCCGTCATCGTTAATTACA
CATCCAGAATGCTCGCCGACAGTTATCGATCCCCGGAGGGAACACGCAACTATAC
TTACATGGACGTTGTCCGAGTCTACCTTGGTGGTAGGAAAGTGCAGCTATGTGGA
CTGGCACAGTTTGGGAGTCTCGTAGGGGTTACTATTGGTTACACCATCACTGCCT
CCATAAGCTTAGTGGCGATTGGGAAAGCAAATTGTTTTCATGACAAGGGACATGG
TGCGAAATGTTCCGTATCAAATTATCCACTCATGGCGGCGTTTGGGATCGTCCAG
ATTTTTCTTAGTCAGATTCCTAATTTTCACAAGCTCTCTTTTCTCTCCATTATCGCCA
CCGTTATGTCCTTCTCTTATGCATCTATCGGTTTTGGCTTAGCCTTGGCCGCTCTG
GCAAGTGGGAAGGTTGGTAAGACGGGACTGACAGGCACGGTGGTTCGAGTGGAC
GTAACTGCGTCTGACAAATTATGGAAGTCATTTCAAGCGGCTGGAAACATTGCCTT
TTCATACGCTTATTCCGTTGTTCTCGTTGAGATTCAGGACACACTGAGATCAAGCC
CACCAGAGAACAAAGTCATGAAAAAAGCAAGCCTTGCTGCAGTCTCAACTACAAC
TGCTTTCTACATCTTATGTGGCTGCATCGGATATGCTACATTTGGAAACCAAGCCC
CCGGAGACTTCCTTACTGACTTTGGTTTTTATGAACCTTACTGGCTCATCGATTTT
GCTAATGCTTGCATCGCTGTCCACCTTATCGGAGCTTATCAGGTGTTTGCACAACC
AATATTCCAGTTTGTTGAGAAGAAATGCAATCAGGCGTGGCCAGAAAGCAACTTCA
TCACCAAAGAACCTTCGATGAACGTACCGTTGCTTGGAAAATGTCGCATTAACTTC
TTCAGACTGGTGTGGAGGACAACCTATGTGATTTTCTCAACAGTTGTAGCAATGAT

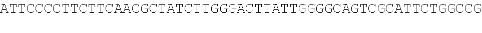

CTAACAGTTTACTTCCCGGTGGAGATGCACATCTCGCAGAAAAAGGTTAAGAAGT
ATTCGGTGAGATGGATAGTGTTGAAACTCCTTGTTTTGGTTTGTTTAATTGTTTCAC
TCCTAGCTGCCATAGGATCCATCGTTGGCTTGATAAGTAGTGTCAAGGCATACAA
GCCTTTCCACAATTTAGATTAG
BRASSICA CRETICA
SEQ ID NO: 165: RQL92522.1 hypothetical protein DY000_00018764
[Brassica cretical] (protein)
MKTFHTEYSPSAVETAGNNFDDDGREKRTGTLMTATAHIITAVIGSGVLSLAWAIAQL
GWVAGTVILVTFAVINYFTSTMLADCYRSPDTGIRNYNYMDVVRAYLGGWKVKLCGL
AQYGSLVGITIGYTITASISLVAIGKANCFHEKGHGAKCSVSNYPLMAAFGIIQIVLSQIH
NFHKLSFLSIIATVMSFSYASVGIGLALAALASGKVGKTDLTGTVVGVDVTASDKIWKS
FQAAGDIAFSYAFSVDTLRSSPPENKVMKKASLAGVSTTTGFYILCGCIGYAAFGNQA
PDFLTDFGFYEPYWLIDFANACIAVHLIAAYQVFAQPIFQFIEKKCNKAWPESNFITKD

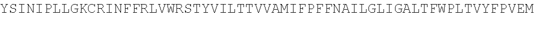

HISQKKVKKYTMRWIGLKLLVLVCLVVSLLAAIGSIVGLISSVKAYKPFHNLD
SEQ ID NO: 166: RQL92522. hypothetical protein DY000_00018764
[Brassica cretical] (genomic)
ATGAAAACCTTCCACACTGAGTATAGTCCCTCGGCCGTGGAAACCGCCGGGAATA
ACTTCGACGATGATGGTCGGGAGAAGAGAACGGGGACGTTGATGACGGCGACCG
CGCACATAATCACGGCGGTGATAGGTTCTGGAGTCTTGTCGTTGGCTTGGGCTAT
AGCACAACTTGGTTGGGTGGCAGGAACGGTGATTTTGGTAACTTTTGCCGTTATA
AATTACTTCACATCTACAATGCTTGCCGACTGTTATCGATCCCCGGACACAGGAAT
ACGTAATTATAATTACATGGACGTTGTCCGAGCTTACCTTGGTGGTTGGAAGGTAA
AGTTATGTGGACTGGCACAGTACGGGAGTCTAGTAGGGATTACTATTGGTTATAC
CATCACTGCCTCCATAAGCTTAGTAGCGATCGGGAAAGCAAATTGTTTTCATGAAA
AGGGACATGGTGCAAAATGTTCCGTATCAAATTATCCACTCATGGCGGCGTTTGG
TATCATCCAGATTGTTCTTAGTCAAATTCATAATTTTCACAAGCTCTCTTTTCTCTCC
ATTATCGCCACGGTTATGTCCTTCTCTTATGCATCTGTCGGAATTGGCTTAGCCTT
GGCCGCTCTGGCAAGTGGGAAGGTTGGTAAGACGGATCTGACGGGCACGGTGG
TTGGAGTAGACGTAACTGCGTCTGACAAAATATGGAAGTCATTCCAAGCAGCTGG
AGACATTGCCTTTTCGTATGCATTTTCCGTTGATACACTGAGATCAAGCCCACCAG
AGAACAAAGTCATGAAAAAAGCAAGCCTTGCTGGAGTTTCAACTACAACTGGTTTC
TACATCTTATGTGGCTGCATCGGATATGCTGCTTTTGGAAACCAAGCCCCTGGAG
ACTTCCTAACTGACTTTGGTTTTTATGAGCCTTACTGGCTCATTGATTTTGCTAATG
CTTGCATTGCTGTCCACCTAATCGCAGCCTATCAGGTGTTTGCACAACCAATATTC
CAGTTTATTGAGAAGAAATGCAACAAAGCGTGGCCAGAAAGCAACTTTATCACCAA
AGATTATTCGATAAACATACCATTGCTAGGGAAATGTCGCATCAACTTCTTCAGATT
GGTCTGGAGGTCAACCTATGTGATTTTGACAACAGTTGTAGCAATGATATTCCCCT

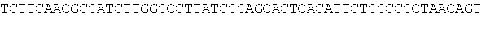

TTACTTCCCAGTGGAGATGCACATCTCGCAGAAAAAGGTTAAGAAGTATACTATGA
GATGGATAGGGTTGAAACTCCTTGTATTGGTTTGTTTGGTTGTTTCGCTCCTAGCT
GCCATAGGATCCATCGTTGGCTTGATAAGTAGTGTAAAGGCATACAAGCCTTTCCA
CAATTTAGATTAG
SEQ ID NO: 169: MUM4 promoter
gacggtggcattaagcatcttgcattgaatgatccgttatatataatctcaggttttttttgggttgaaatgatgatatt
aaattttaggttgacatgtacttatctttgtaatcaactaattaaatatttgaactgacatgtctacgttatatcataaat
aaaccaggtgttttaattaaataccacgattaaccttctaaaataaggaaaatcatattttattcgtcaatcactata
atttggaaaacgatgcaatatatttatttctttctttatacacatacttaattaattatcaaaatttc

### REFERENCES

1. Adamski, N. M., Anastasiou, E., Eriksson, S., O'Neill, C. M., and Lenhard, M. (2009). Local maternal control of seed size by KLUH/CYP78A5-dependent growth signaling. Proc Natl Acad Sci USA 106, 20115-20120.
2. Alonso-Blanco, C., Blankestijn-de Vries, H., Hanhart, C. J., and Koornneef, M. (1999). Natural allelic variation at seed size loci in relation to other life history traits of Arabidopsis thaliana. Proc Natl Acad Sci USA 96, 4710-4717.
3. Clough S J, Bent A F. Floral dip: a simplified method for Agrobacterium-mediated transformation of Arabidopsis thaliana. Plant J.: Cell Mol. Biol. 1998; 16:735-743
4. Du, L., Li, N., Chen, L., Xu, Y., Li, Y., Zhang, Y., Li, C., and Li, Y. (2014). The ubiquitin receptor DA1 regulates seed and organ size by modulating the stability of the ubiquitin-specific protease UBP15/SOD2 in Arabidopsis. Plant Cell 26, 665-677.
5. Garcia, D., Fitz Gerald, J. N., and Berger, F. (2005). Maternal control of integument cell elongation and zygotic control of endosperm growth are coordinated to determine seed size in Arabidopsis. Plant Cell 17, 52-60.
6. Gaudelli N. M.; Komor A. C.; Rees H. A.; Packer M. S.; Badran A. H.; Bryson D. I.; Liu D. R. Programmable base editing of A-T to G-C in genomic DNA without DNA cleavage. Nature 2017, 551, 464-47110.1038/nature24644
7. Keurentjes, J. J., Bentsink, L., Alonso-Blanco, C., Hanhart, C. J., Blankestijn-De Vries, H., Effgen, S., Vreugdenhil, D., and Koornneef, M. (2007). Development of a near-isogenic line population of Arabidopsis thaliana and comparison of mapping power with a recombinant inbred line population. Genetics 175, 891-905.
8. Kim, Y. B. et al. Increasing the genome-targeting scope and precision of base editing with engineered Cas9-cytidine deaminase fusions. Nat. Biotechnol. 35, 371-376 (2017)
9. Lee, Y. H., Foster, J., Chen, J., Voll, L. M., Weber, A. P., and Tegeder, M. (2007). AAP1 transports uncharged amino acids into roots of Arabidopsis. Plant J 50, 305-319.
10. Li, Y. H., Zheng, L. Y., Corke, F., Smith, C., and Bevan, M. W. (2008b). Control of final seed and organ size by the DA1 gene family in Arabidopsis thaliana. Gene Dev 22, 1331-1336.
11. Li, N., and Li, Y. (2016). Signaling pathways of seed size control in plants. Curr Opin Plant Biol 33, 23-32.
12. Li B, Zhao W, Luo X, Zhang X, Li C, Zeng C, et al. Engineering CRISPR-Cpf1 crRNAs and mRNAs to maximize genome editing efficiency. Nat Biomed Eng. 2017; 1(5):0066
13. Ma, X. and Liu, Y.-G. (2016) CRISPR/Cas9-based multiplex genome editing in monocot and dicot plants. Curr. Protoc. Mol. Biol. 115, 31.6.1-31.6.21
14. Okumoto, S., Schmidt, R., Tegeder, M., Fischer, W. N., Rentsch, D., Frommer, W. B., Koch, W. (2002). Highly affinity amino acid transporters specifically expressed in xylem parenchyma and developing seeds of Arabidopsis. J BIOL CHEM 277, 45338-45346.
15. Peng, B., Kong, H., Li, Y., Wang, L., Zhong, M., Sun, L., Gao, G., Zhang, Q., Luo, L., Wang, G., et al. (2014). OsAAP6 functions as an important regulator of grain protein content and nutritional quality in rice. Nat Commun 5, 4847.
16. Sambrook, et al., (1989) Molecular Cloning: A Library Manual (2d ed., Cold Spring Harbor Laboratory Press, Plainview, N.Y.).
17. Sanders, A., Collier, R., Trethewy, A., Gould, G., Sieker, R., Tegeder, M. (2009). AAP1 regulates import of amino acids into developing. Plant J 59, 540-552
18. Santiago, J. P., and Tegeder, M. (2016). Connecting Source with Sink: The Role of Arabidopsis AAP8 in Phloem Loading of Amino Acids. Plant Physiol 171, 508-521.
19. Schmidt, R., Stransky, H., and Koch, W. (2007). The amino acid permease AAP8 is important for early seed development in Arabidopsis thaliana. Planta 226, 805-813.
20. Schruff, M. C., Spielman, M., Tiwari, S., Adams, S., Fenby, N., and Scott, R. J. (2006). The AUXIN RESPONSE FACTOR 2 gene of Arabidopsis links auxin signalling, cell division, and the size of seeds and other organs. Development 133, 251-261.
21. Song, X. J., Huang, W., Shi, M., Zhu, M. Z., and Lin, H. X. (2007). A QTL for rice grain width and weight encodes a previously unknown RING-type E3 ubiquitin ligase. Nat Genet 39, 623-630.
22. Tan, H., Yang, X., Zhang, F., Zheng, X., Qu, C., Mu, J., Fu, F., Li, J., Guan, R., Zhang, H., et al. (2011). Enhanced seed oil production in canola by conditional expression of Brassica napus LEAFY COTYLEDON1 and LEC1-LIKE in developing seeds. Plant Physiol 156, 1577-1588.
23. Tegeder, M. (2012). Transporters for amino acids in plant cells: some functions and many unknowns. Curr Opin Plant Biol 15, 315-321.
24. Wang, L., Lu, Q., Wen, X., and Lu, C. (2015). Enhanced Sucrose Loading Improves Rice Yield by Increasing Grain Size. Plant Physiol 169, 2848-2862.
25. Wiles M V, Qin W, Cheng A W, Wang H. CRISPR-Cas9-mediated genome editing and guide RNA design. Mamm Genome. 2015; 26(9):501-510
26. Xia, T., Li, N., Dumenil, J., Li, J., Kamenski, A., Bevan, M. W., Gao, F., and Li, Y. (2013). The ubiquitin receptor DA1 interacts with the E3 ubiquitin ligase DA2 to regulate seed and organ size in Arabidopsis. Plant Cell 25, 3347-3359.
27. Zhang, Y., Du, L., Xu, R., Cui, R., Hao, J., Sun, C., and Li, Y. (2015). Transcription factors SOD7/NGAL2 and DPA4/NGAL3 act redundantly to regulate seed size by directly repressing KLU expression in Arabidopsis thaliana. Plant Cell 27, 620-632.

1. A method of increasing seed yield in a plant, the method comprising increasing the activity of amino acid permease (AAP).

2. (canceled)

3. The method of claim 1, wherein the method comprises increasing the expression of AAP8, wherein the amino acid sequence of AAP8 comprises a sequence as defined in SEQ ID NO: 2, 3 or 4 or a functional variant or homologue thereof.

4. The method of claim 3, wherein the method comprises introducing and expressing a nucleic acid construct, wherein the construct comprises a nucleic sequence encoding an AAP8 polypeptide as defined in SEQ ID NO: 2, 3 or 4 or a functional variant or homologue thereof, wherein the nucleic acid sequence is operably linked to a regulatory sequence, wherein the regulatory sequence is a constitutive or tissue-specific promoter, and wherein preferably the tissue-specific promoter is a MUM4 promoter.

5. (canceled)

6. (canceled)

7. (canceled)

8. The method of claim 1, wherein the method comprises introducing at least one mutation into the plant genome, wherein said mutation increases the activity of an AAP polypeptide.

9. (canceled)

10. (canceled)

11. The method of claim 8, wherein the mutation is the insertion of at least one additional copy of a nucleic acid sequence encoding an AAP8 polypeptide or a homolog or functional variant thereof, such that the nucleic acid sequence is operably linked to a regulatory sequence, and wherein the mutation is introduced using targeted genome editing and wherein preferably the nucleic acid sequence encodes an AAP polypeptide as defined in SEQ ID NO: 2, 3 or 4 or a functional variant or homolog thereof; or wherein the method comprises or results in introducing at least one mutation at position 410 of SEQ ID NO: 1 or at a homologous position in a homologous sequence.

12. (canceled)

13. (canceled)

14. The method of claim 1, wherein the plant is a crop plant, wherein preferably, the crop plant is selected from rice, maize, wheat, soybean, barley, cannabis and pennycress and Brassica.

15. (canceled)

16. A plant or plant progeny obtained or obtainable by the method of any of claim 1.

17. A genetically altered plant, part thereof or plant product, wherein the plant is characterized by an increase in seed yield, wherein the plant has increased activity of an AAP polypeptide.

18. (canceled)

19. The genetically altered plant of claim 17, wherein the plant expresses a nucleic acid construct comprising a nucleic acid encoding an AAP8 polypeptide as defined in any of SEQ ID NO: 2, 3 or 4 or a functional variant or homologue thereof.

20. The genetically altered plant of claim 17, wherein the plant has at least one mutation in its genome, wherein the mutation increases the activity of AAP8, wherein the mutation is in the insertion of at least one or more additional copy of a nucleic acid encoding an AAP8 polypeptide as defined in SEQ ID NO: 2, 3 or 4 or homolog or functional variant thereof; or wherein the at least one mutation is at position 410 of SEQ ID NO: 1 or at a homologous position in a homologous sequence.

21. (canceled)

22. (canceled)

23. (canceled)

24. The genetically altered plant of claim 17, wherein the plant is a crop plant, preferably wherein the crop plant is selected from rice, maize, wheat, soybean, barley, cannabis and pennycress and Brassica.

25. (canceled)

26. The genetically altered plant of claim 17, wherein the plant part is a seed.

27. A method of making a transgenic plant having an increase in seed yield, the method comprising introducing and expressing a nucleic acid construct comprising a nucleic acid sequence encoding an AAP8 polypeptide as defined in SEQ ID NO: 2, 3 or 4 or a functional variant or homolog thereof.

28. A method of making a genetically altered plant having an increase in seed yield, the method comprising introducing a mutation into the plant genome to increase the activity of an AAP8 polypeptide, wherein the mutation is the insertion of one or more additional copies of a nucleic acid encoding an AAP8 polypeptide as defined in SEQ ID NO: 2, 3 or 4 or a functional variant or homolog thereof, such that the sequence is operably linked to a regulatory sequence; or wherein the method comprises or results in introducing at least one mutation at position 410 of SEQ ID NO: 1 or at a homologous position in a homologous sequence.

29. (canceled)

30. (canceled)

31. (canceled)

32. (canceled)

33. The method of claim 27, wherein the plant is a crop plant, wherein preferably the crop plant is selected from rice, maize, wheat, soybean, barley, cannabis, pennycress and Brassica.

34. (canceled)

35. A method of screening a population of plants and identifying and/or selecting a plant that has or will have increased activity of a AAP polypeptide, the method comprising detecting in the plant germplasm at least one polymorphism in the nucleic acid encoding an AAP polypeptide and selecting said plant or progeny thereof.

36. (canceled)

37. The method of claim 35, wherein the polymorphism is a substitution and wherein the substitution is at position 2635 of SEQ ID NO: 5 or a homologous substitution in a homologous sequence.

38. A nucleic acid construct comprising a nucleic acid sequence encoding a AAP8 polypeptide as defined in SEQ ID NO: 2, 3 or 4 or a functional variant or homolog thereof.

39. (canceled)

40. (canceled)

41. (canceled)

42. (canceled)

43. A method of producing a food or feed composition, the method comprising
a. producing a plant wherein the activity of an AAP polypeptide is increased using the method defined in claim 27;
b. obtaining a seed from said plant; and
c. producing a food or feed composition from said seed.

44. A method of increasing free amino acid and/or protein content in a plant, preferably increasing free amino acid and/or protein content in the seed or grain of said plant, the method comprising increasing the activity of amino acid permease (AAP), wherein, the method comprises increasing the activity and/or expression of AAP8, wherein the amino acid sequence of AAP8 comprises a sequence as defined in SEQ ID NO: 2, 3 or 4 or a functional variant or homologue thereof.

45. (canceled)
